(19) United States
(12) Patent Application Publication
(10) Pub. No.: US 2011/0207704 A1
(43) Pub. Date: Aug. 25, 2011

(54) Novel Oxadiazole Compounds

(75) Inventors: Kevin P. Cusack, Holden, MA (US); Eric C. Breinlinger, Charlton, MA (US); Shannon R. Fix-Stenzel, Chicago, IL (US); Robert H. Stoffel, Harvard, MA (US); Kevin R. Woller, Anitoch, IL (US); Adrian D. Hobson, Shrewsbury, MA (US); Pintipa Grongsaard, Whippany, NJ (US)

(73) Assignee: ABBOTT LABORATORIES, Abbott Park, IL (US)

(21) Appl. No.: 12/861,931

(22) Filed: Aug. 24, 2010

Related U.S. Application Data
(63) Continuation-in-part of application No. 12/002,196, filed on Dec. 14, 2007, now Pat. No. 7,834,039.
(60) Provisional application No. 60/875,251, filed on Dec. 15, 2006.
(60) Provisional application No. 61/267,679, filed on Dec. 8, 2009.

(51) Int. Cl. A61K 31/675; C07D 271/06; A61K 31/4245; C07D 413/12; C07D 413/10; C07D 413/04; A61K 31/4439; C07D 401/14; A61K 31/439; C07F 9/40; A61K 31/4709; A61K 31/454; A61K 31/5377; A61K 31/397; C07D 487/04; A61K 31/4985; A61P 11/00; A61P 31/14; A61P 25/28; A61P 17/06; A61P 19/02; A61P 1/04

(52) U.S. Cl. 514/92; 548/131; 514/364; 546/269.4; 514/340; 546/183; 514/299; 548/119; 546/166; 514/314; 546/209; 514/326; 544/138; 514/236.2; 514/210.18; 544/350; 514/249

(57) ABSTRACT

Novel oxadiazole compounds, pharmaceutical compositions containing such compounds and the use of those compounds or compositions as agonists or antagonists of the S1P family of G protein-coupled receptors for treating diseases associated with modulation of S1P family receptor activity, in particular by affording a beneficial immunosuppressive effect are disclosed.

### CROSS REFERENCE TO RELATED APPLICATION

This application is a continuation in part application claiming priority to U.S. application Ser. No. 12/002,196, filed Dec. 14, 2007, which is a non-provisional application that claims priority to U.S. Provisional Application Ser. No. 60/875,251 filed on Dec. 15, 2006, the contents of which are incorporated herein. This application also claims priority to U.S. Provisional Application Ser. No. 61/267,679 filed on Dec. 8, 2009, the contents of which are incorporated herein.

### BACKGROUND OF THE INVENTION

Sphingosine-1-phosphate (S1P) is part of sphingomyelin biosynthetic pathway and is known to affect multiple biological processes. S1P is formed through phosphorylation of sphingosine by sphingosine kinases (SK1 and SK2) and it is degraded through cleavage by sphingosine lyase to form palmitaldehyde and phosphoethanolamine or through dephosphorylation by phospholipid phosphatases. It is present at high levels (˜500 nM) in serum and it is found in most tissues. It can be synthesized in a wide variety of cells in response to several stimuli, which include cytokines, growth factors and G protein-coupled receptor (GPCR) ligands. The GPCRs that bind S1P (currently know as the S1P receptors S1P1-5), couple through pertusis toxin sensitive (Gi) pathways as well as pertusis toxin insensitive pathways to stimulate a variety of processes. The individual receptors of the S1P family are both tissue and response specific and so are attractive as therapeutic targets.

S1P evokes many responses from cells and tissues. In particular, S1P has been shown to be an agonist at all five GPCRs, S1P1 (Edg-1), S1P2 (Edg-5), S1P3 (Edg-3), S1P4 (Edg-6) and S1P5 (Edg-8). The action of S1P at the S1P receptors has been linked to resistance to apoptosis, changes in cellular morphology, cell migration, growth, differentiation, cell division, angiogenesis and modulation of the immune system via alterations of lymphocyte trafficking. Therefore, S1P receptors are targets for therapy of, for example, neoplastic diseases, autoimmune disorders and tissue rejection in transplantation. These receptors also share 50-55% amino acid identity with three other lysophospholipid receptors, LPA1, LPA2, and LPA3 of the structurally related lysophosphatidic acid (LPA).

GPCRs are excellent drug targets with numerous examples of marketed drugs across multiple disease areas. GPCRs are cell surface receptors that bind hormones on the extracellular surface of the cell and transduce a signal across the cellular membrane to the inside of the cell. The internal signal is amplified through interaction with G proteins which in turn interact with various second messenger pathways. This transduction pathway is manifested in downstream cellular responses that include cytoskeletal changes, cell motility, proliferation, apoptosis, secretion and regulation of protein expression to name a few. SIP receptors make good drug targets because individual receptors are expressed in different tissues and signal through different pathways making the individual receptors both tissue and response specific. Tissue specificity of the SIP receptors is desirable because development of an agonist or antagonist selective for one receptor localizes the cellular response to tissues containing that receptor, limiting unwanted side effects. Response specificity of the SIP receptors is also of importance because it allows for the development of agonists or antagonists that initiate or suppress certain cellular responses without affecting other responses. For example, the response specificity of the SIP receptors could allow for an SIP mimetic that initiates platelet aggregation without affecting cell morphology.

The physiologic implications of stimulating individual SIP receptors are largely unknown due in part to a lack of receptor type selective ligands. Isolation and characterization of SIP analogs that have potent agonist or antagonist activity for SIP receptors have been limited.

S1P1 for example is widely expressed and the knockout causes embryonic lethality due to large vessel rupture. Adoptive cell transfer experiments using lymphocytes from S1P1 knockout mice have shown that S1P1 deficient lymphocytes sequester to secondary lymph organs. Conversely, T cells overexpressing S1P1 partition preferentially into the blood compartment rather than secondary lymph organs. These experiments provide evidence that S1P1 is the main sphingosine receptor involved in lymphocyte homing and trafficking to secondary lymphoid compartments

Currently, there is a need for novel, potent, and selective agents, which are agonists or antagonists of the individual receptors of the S1P receptor family in order to address unmet medical needs associated with agonism or antagonism of the individual receptors of the S1P receptor family.

### SUMMARY OF THE INVENTION

The present invention provides novel compounds described by general Formula (I), (Ia), (II), (III), (IV), (IVa), (IVb) or (V) as agonists of the G protein-coupled receptor S1P1. These compounds reduce the number of circulating and infiltrating T- and B-lymphocytes affording a beneficial immunosuppressive effect. The compounds also exhibit activity within the S1P receptor family.

In a first embodiment the invention provides a compound of Formula (I)

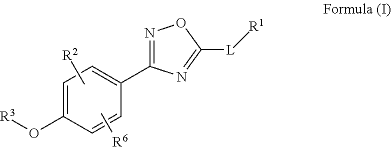

pharmaceutically acceptable salts, biologically active metabolites, solvates, hydrates, prodrugs, racemates, enantiomers or stereoisomers thereof, wherein

L is a bond or optionally substituted (C₁-C₃)alkyl;

R¹ is —C(O)—NH-phenyl, —NH—C(O)-furanyl, —NH—S(O)₂-optionally substituted phenyl, —O-optionally substituted (C₁-C₃)alkyl, —S-optionally substituted (C₁-C₃)alkyl, optionally substituted (C₂-C₆)alkyl, optionally substituted amino, optionally substituted (C₃-C₆)cycloalkyl, —(CH₂)(C₃)alkyl, optionally substituted tetrahydrobenzofuranyl, optionally substituted furanyl, optionally substituted tetrahydrofuranyl, optionally substituted 2,3-dihydroisoindolyl, optionally substituted isoindolinyl, optionally substituted imidazolyl, optionally substituted 5,6-dihydro imidazo[1,2-a]pyrazinyl, optionally substituted imidazo[1,2-a]pyrazinyl, optionally substituted indolyl, optionally substituted isoxazolyl, optionally substituted morpholinyl, optionally substituted naphthyl, optionally substituted phenyl, —O—CH₂-optionally substituted phenyl, —O-optionally substituted phenyl, —O-optionally substituted phenyl, optionally substituted piperidinyl, optionally substituted pyrazolyl, optionally substituted pyridinyl, optionally substituted pyrimidinyl, optionally substituted pyrrolidinyl, optionally substituted 1,2,3,4-tetrahydroisoquinolinyl, optionally substituted quinolinyl, optionally substituted 3,4-dihydroquinolinyl, optionally substituted 3,4-dihydroisoquinolinyl, optionally substituted 5,6,7,8-tetrahydroimidazo[1,2-a]pyrazinyl, optionally substituted pyrrolyl, optionally substituted pyrrolo[2,3-b]pyridinyl, optionally substituted quinolinyl, optionally substituted thiazolyl or optionally substituted thienyl;

R² is Br, C₁, CF₃, CN, or —O—(C₁-C₂)alkyl;

R³ is optionally substituted-(C₃-C₈)alkyl, deuterated —(C₂-C₆)alkyl, (C₄-C₅)alkenyl, (C₄-C₅)alkynyl, optionally substituted-(C₃-C₆)cycloalkyl, -optionally substituted (C₂-C₃)alkyl-O-optionally substituted (C₁-C₃)alkyl, -optionally substituted (C₁-C₃)alkyl-imidazolyl, -optionally substituted (C₁-C₃)alkyl-morpholinyl, -optionally substituted (C₁-C₃)alkyl-optionally substituted phenyl, -optionally substituted (C₁-C₃)alkyl-optionally substituted piperazinyl, -optionally substituted (C₁-C₃)alkyl-pyrrolidinyl, -optionally substituted (C₁-C₃)alkyl-piperidinyl, optionally substituted (C₁-C₃)alkyl-thienyl, tetrahydrofuranyl or optionally substituted thiazolyl; and

R⁶ is H;

provided that

R¹ is not substituted by optionally substituted cyclohexyl, —C(O)-cyclohexyl or —NH-cyclohexyl;

when L is (C₁-C₃)alkyl, R¹ is not optionally substituted isoxazolyl;

when R³ is optionally substituted (C₁)alkyl, L-R¹ is not cyclohexyl or —CH₂-cyclohexyl; and

provided that the compound is not

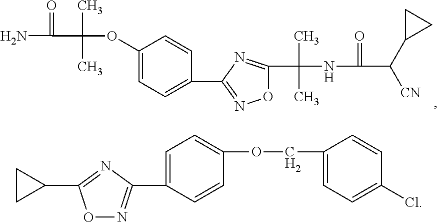

In a second embodiment the invention provides the compound according to the first embodiment wherein R¹ is optionally substituted by one or more substituents independently selected from Br, Cl, F, CF₃, CN, oxo, —C(═O)H, —N(R⁹)₂, optionally substituted (C₁-C₆)alkyl, optionally substituted (C₂-C₆)alkenyl, optionally substituted (C₂-C₆)alkynyl, optionally substituted azabicyclo[2.2.1]heptanyl, optionally substituted (C₃-C₆)cycloalkyl, —C(R⁹)₂-optionally substituted (C₃-C₆)cycloalkyl, —C(R⁹)₂-optionally substituted azetidinyl, —CR⁹₂-optionally substituted piperidinyl, —C(R⁹)₂-optionally substituted pyrrolidinyl, —C(R⁹)₂—N(R⁹)₂, —C(O)-optionally substituted (C₁-C₆)alkyl, —C(O)—NR⁹—(C₁-C₆)alkyl, —C(O)—O-optionally substituted (C₁-C₆)alkyl, —C(R⁹)₂—C(O)—O-optionally substituted (C₁-C₆)alkyl, —NR⁹-optionally substituted (C₃-C₆)cycloalkyl, —NR⁹-optionally substituted azetidinyl, —NR₉-furanyl, —NR⁹-optionally substituted pyrrolidinyl, —NR⁹—C(O)—O-optionally substituted (C₁-C₃)alkyl, —NR⁹-optionally substituted (C₁-C₆)alkyl, —NR⁹-optionally substituted (C₃-C₆)cycloalkyl, —NR⁹—C(O)-azetidinyl, —NR⁹—C(O)-furanyl, —NR⁹—C(O)-pyridinyl, —NR⁹—C(O)-optionally substituted pyrrolidinyl, —NR⁹—S(O)₂-optionally substituted phenyl, —O-optionally substituted (C₁-C₆)alkyl, —O-deuterated —(C₂-C₆)alkyl, —O-optionally substituted (C₂-C₆)alkenyl, —O-optionally substituted (C₃-C₆)cycloalkyl, —O-1H-benzo[d][1,2,3]triazolyl, —S(O)₂—N(R⁹)₂, —S(O)₂—NR⁹-optionally substituted (C₁-C₄)alkyl, optionally substituted azetidinyl, optionally substituted piperidinyl, optionally substituted pyridinyl, optionally substituted pyrrolidinyl, optionally substituted 1,2,4 oxadiazolyl, optionally substituted pyrrolidinyl, optionally substituted tetrazolyl,

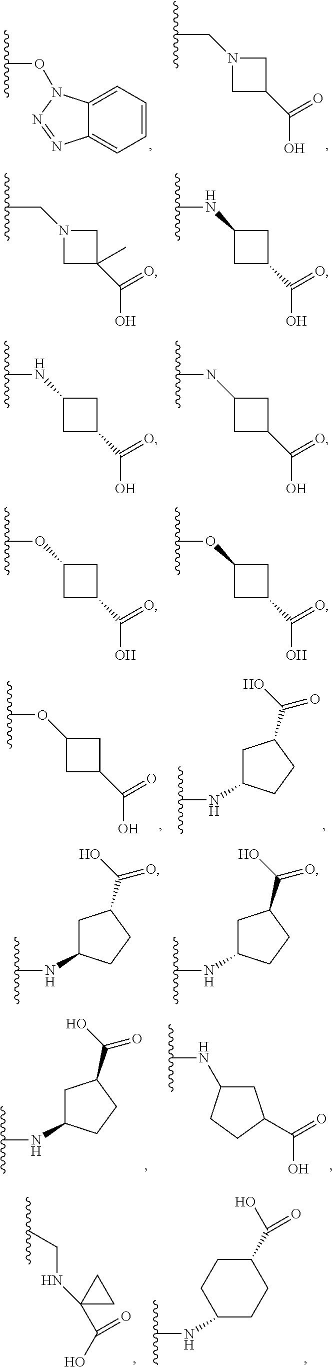

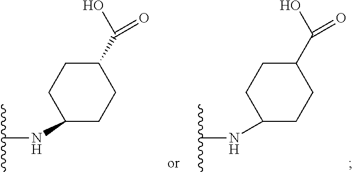

and

wherein each R⁹ is independently selected from H or optionally substituted (C₁-C₆)alkyl.

In a third embodiment the invention provides a compound according to any of the foregoing embodiments wherein the compound is a compound of Formula (Ia)

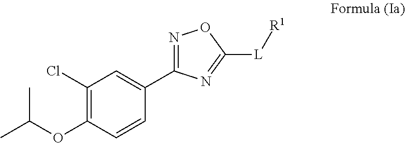

wherein L is a bond.

In a fourth embodiment the invention provides a compound according to any of the foregoing embodiments wherein R¹ is optionally substituted tetrahydrobenzofuranyl, optionally substituted furanyl, optionally substituted 2,3-dihydroisoindolyl, optionally substituted isoindolinyl, optionally substituted imidazolyl, optionally substituted 5,6-dihydro imidazo[1,2-a]pyrazinyl, optionally substituted imidazo[1,2-a]pyrazinyl, optionally substituted indolyl, optionally substituted isoxazolyl, optionally substituted pyrazolyl, optionally substituted pyridinyl, optionally substituted pyrimidinyl, optionally substituted pyrrolidinyl, optionally substituted 1,2,3,4-tetrahydroisoquinolinyl, optionally substituted quinolinyl, optionally substituted 3,4-dihydroquinolinyl, optionally substituted 3,4-dihydroisoquinolinyl, optionally substituted 5,6,7,8-tetrahydroimidazo[1,2-a]pyrazinyl, optionally substituted pyrrolyl, optionally substituted pyrrolo[2,3-b]pyridinyl, optionally substituted quinolinyl, optionally substituted thiazolyl or optionally substituted thienyl.

In a fifth embodiment the invention provides a compound according to any of the foregoing embodiments wherein R¹ is optionally substituted furanyl, optionally substituted imidazolyl, optionally substituted isoxazolyl, optionally substituted pyrazolyl, optionally substituted pyridinyl, optionally substituted pyrimidinyl, optionally substituted pyrrolidinyl, optionally substituted pyrrolyl, optionally substituted thiazolyl or optionally substituted thienyl.

In a sixth embodiment the invention provides a compound according to any of the foregoing embodiments wherein R¹ is optionally substituted phenyl or optionally substituted indolyl.

In a seventh embodiment the invention provides a compound of according to any of the foregoing embodiments wherein

L is optionally substituted (C₁-C₃)alkyl;

R¹ is —C(O)—NH-phenyl, —NH—C(O)-furanyl, —NH—S(O)₂-optionally substituted phenyl, optionally substituted —O—(C₁-C₃)alkyl, —S—(C₁-C₃)alkyl, optionally substituted benzyloxy, optionally substituted (C₃-C₆)cycloalkyl, optionally substituted imidazolyl, morpholinyl, optionally substituted naphthyl, optionally substituted phenyl, optionally substituted phenoxy, optionally substituted piperazinyl, optionally substituted piperidinyl, optionally substituted pyridinyl, optionally substituted pyrrolidinyl or optionally substituted thienyl;

R² is Cl;

R³ is isopropyl; and

R⁶ is H.

In an eighth embodiment the invention provides a compound of according to any of the foregoing embodiments wherein L is CH₂ and R¹ is optionally substituted phenyl or optionally substituted (C₃-C₆)cycloalkyl.

In a ninth embodiment the invention provides a compound of according to any of the foregoing embodiments wherein the compound is
4-[3-(3-chloro-4-isopropoxy-phenyl)-[1,2,4]oxadiazol-5-yl]-3-methyl-pyridine;
3-[3-chloro-4-(1-ethyl-propoxy)-phenyl]-5-o-tolyl-[1,2,4]oxadiazole;
3-(3-chloro-4-isopropoxyphenyl)-5-(3-chloropyridin-4-yl)-[1,2,4]-oxadiazole;
3-chloro-4-(3-(3-chloro-4-isopropoxyphenyl)-1,2,4-oxadiazol-5-yl)benzonitrile;
1-(3-chloro-4-(3-(3-chloro-4-isopropoxyphenyl)-1,2,4-oxadiazol-5-yl)benzyl)-3-methylazetidine-3-carboxylic acid;
tert-butyl 3-(4-(3-(3-chloro-4-isopropoxyphenyl)-1,2,4-oxadiazol-5-yl)-1H-indol-1-yl)propanoate;
tert-butyl 4-(4-(3-(3-chloro-4-isopropoxyphenyl)-1,2,4-oxadiazol-5-yl)-1H-indol-1-yl)butanoate;
3-(4-(3-(3-chloro-4-isopropoxyphenyl)-1,2,4-oxadiazol-5-yl)-1H-indol-1-yl)propanoic acid;
(1R,3S)-3-(4-(3-(3-chloro-4-isopropoxyphenyl)-1,2,4-oxadiazol-5-yl)phenylamino)cyclopentanecarboxylic acid;
3-(4-(3-(3-chloro-4-isopropoxyphenyl)-1,2,4-oxadiazol-5-yl)phenoxy)propane-1,2-diol;
(R)-3-{3-chloro-4-[3-(3-chloro-4-isopropoxy-phenyl)-[1,2,4]oxadiazol-5-yl]-phenoxy}-propane-1,2-diol;
3-{3-chloro-4-[5-(5-chloro-6-isopropoxy-pyridin-3-yl)-[1,2,4]oxadiazol-3-yl]-phenoxy}-cyclobutanecarboxylic acid;
3-(4-(3-(3-chloro-4-isopropoxyphenyl)-1,2,4-oxadiazol-5-yl)phenylamino)propylphosphonic acid;
ethyl 3-(4-(3-(3-chloro-4-isopropoxyphenyl)-1,2,4-oxadiazol-5-yl)benzylidene)cyclobutanecarboxylate;
ethyl 3-(4-(3-(3-chloro-4-isopropoxyphenyl)-1,2,4-oxadiazol-5-yl)benzyl)cyclobutanecarboxylate;
3-(4-(3-(3-chloro-4-isopropoxyphenyl)-1,2,4-oxadiazol-5-yl)benzyl)cyclobutanecarboxylic acid;
5-(3-(3-chloro-4-isopropoxyphenyl)-1,2,4-oxadiazol-5-yl)-1-methyl-1H-pyrazol-3-amine;
3-(3-chloro-4-isopropoxyphenyl)-5-(1H-indol-5-yl)-1,2,4-oxadiazole;
1-(4-(3-(3-chloro-4-isopropoxyphenyl)-1,2,4-oxadiazol-5-yl)benzyl)pyrrolidine-3-carboxylic acid;
3-amino-1-(4-(3-(3-chloro-4-isopropoxyphenyl)-1,2,4-oxadiazol-5-yl)benzyl)pyrrolidine-3-carboxylic acid;
(S)-1-(4-(3-(3-chloro-4-isopropoxyphenyl)-1,2,4-oxadiazol-5-yl)benzyl)pyrrolidine-3-carboxylic acid;
(R)-1-(4-(3-(3-chloro-4-isopropoxyphenyl)-1,2,4-oxadiazol-5-yl)benzyl)pyrrolidine-3-carboxylic acid;
(S)-1-(4-(3-(3-chloro-4-isopropoxyphenyl)-1,2,4-oxadiazol-5-yl)benzyl)azetidine-2-carboxylic acid;
4-(4-(3-(3-chloro-4-isopropoxyphenyl)-1,2,4-oxadiazol-5-yl)-1H-indol-1-yl)butanoic acid;
3-(4-(3-(3-chloro-4-isopropoxyphenyl)-1,2,4-oxadiazol-5-yl)-1H-indol-1-yl)-2-fluoropropanoic acid;
3-(4-(3-(3-chloro-4-isopropoxyphenyl)-1,2,4-oxadiazol-5-yl)-1H-indol-1-yl)-2-methylpropanoic acid;
3-(4-(3-(3-chloro-4-isopropoxyphenyl)-1,2,4-oxadiazol-5-yl)-1H-indol-1-yl)-2,2-dimethylpropanoic acid;
3-(4-(3-(3-chloro-4-isopropoxyphenyl)-1,2,4-oxadiazol-5-yl)-1H-pyrrolo[2,3-b]pyridin-1-yl)propanoic acid;
(1R,3S)-3-{4-[3-(5-chloro-6-isopropoxy-pyridin-3-yl)-[1,2,4]oxadiazol-5-yl]-3-methyl-phenylamino}-cyclopentanecarboxylic acid;
4-(4-(3-(3-chloro-4-isopropoxyphenyl)-1,2,4-oxadiazol-5-yl)phenylamino)-3,3-dimethylbutanoic acid;
4-(4-(3-(3-chloro-4-isopropoxyphenyl)-1,2,4-oxadiazol-5-yl)phenylamino)butanoic acid;
1-(4-(3-(3-chloro-4-isopropoxyphenyl)-1,2,4-oxadiazol-5-yl)phenyl)pyrrolidine-3-carboxylic acid;
2-(1-(4-(3-(3-chloro-4-isopropoxyphenyl)-1,2,4-oxadiazol-5-yl)phenyl)pyrrolidin-3-yl)acetic acid;
(1R,3S)-3-(4-(3-(3-bromo-4-isopropoxyphenyl)-1,2,4-oxadiazol-5-yl)phenylamino)cyclopentanecarboxylic acid;
(1R,3S)-3-(4-(3-(4-isopropoxy-3-(trifluoromethyl)phenyl)-1,2,4-oxadiazol-5-yl)phenylamino)cyclopentanecarboxylic acid;
(1R,3S)-3-(4-(3-(5-chloro-6-isopropoxypyridin-3-yl)-1,2,4-oxadiazol-5-yl)phenylamino)cyclopentanecarboxylic acid;
(R)-1-(4-(3-(3-chloro-4-isopropoxyphenyl)-1,2,4-oxadiazol-5-yl)phenyl)pyrrolidin-3-amine, acetic acid;
(1R,2S)-2-(4-(3-(3-chloro-4-isopropoxyphenyl)-1,2,4-oxadiazol-5-yl)phenylamino)cyclopentanecarboxylic acid;
(1S,2R)-2-(4-(3-(3-chloro-4-isopropoxyphenyl)-1,2,4-oxadiazol-5-yl)phenylamino)cyclohexanecarboxylic acid;
(S)-1-(3-(4-(3-(3-chloro-4-isopropoxyphenyl)-1,2,4-oxadiazol-5-yl)phenylamino)pyrrolidin-1-yl)ethanone;
(1R,2R)-2-(4-(3-(3-chloro-4-isopropoxyphenyl)-1,2,4-oxadiazol-5-yl)phenylamino)cyclohexanecarboxylic acid;
(1R,2S)-2-(4-(3-(3-chloro-4-isopropoxyphenyl)-1,2,4-oxadiazol-5-yl)phenylamino)cyclohexanecarboxylic acid;
(1R,2S)-2-(4-(3-(3-chloro-4-isopropoxyphenyl)-1,2,4-oxadiazol-5-yl)phenylamino)cyclohexanecarboxylic acid;
(1S,2R)-2-(4-(3-(3-chloro-4-isopropoxyphenyl)-1,2,4-oxadiazol-5-yl)phenylamino)cyclohexanecarboxylic acid;
(1R,2R)-2-(4-(3-(3-chloro-4-isopropoxyphenyl)-1,2,4-oxadiazol-5-yl)phenylamino)cyclohexanecarboxylic acid;
(1S,2S)-2-(4-(3-(3-chloro-4-isopropoxyphenyl)-1,2,4-oxadiazol-5-yl)phenylamino)cyclohexanecarboxylic acid;
(1S,2R)-2-(4-(3-(3-chloro-4-isopropoxyphenyl)-1,2,4-oxadiazol-5-yl)phenylamino)cyclopentanecarboxylic acid;
(1S,2S)-2-(4-(3-(3-chloro-4-isopropoxyphenyl)-1,2,4-oxadiazol-5-yl)phenylamino)cyclohexanecarboxylic acid;
(S)—N-(4-(3-(3-chloro-4-isopropoxyphenyl)-1,2,4-oxadiazol-5-yl)phenyl)-1-(methylsulfonyl)pyrrolidin-3-amine;
(S)-2-(3-(4-(3-(3-chloro-4-isopropoxyphenyl)-1,2,4-oxadiazol-5-yl)phenylamino)pyrrolidin-1-yl)acetic acid;
(1R,3S)-3-(2-bromo-4-(3-(3-chloro-4-isopropoxyphenyl)-1,2,4-oxadiazol-5-yl)phenylamino)cyclopentanecarboxylic acid;
(1R,3S)-3-(2-bromo-3-(3-(3-chloro-4-isopropoxyphenyl)-1,2,4-oxadiazol-5-yl)phenylamino)cyclopentanecarboxylic acid;
(1R,3S)-3-(5-(3-(3-chloro-4-isopropoxyphenyl)-1,2,4-oxadiazol-5-yl)pyridin-2-ylamino)cyclopentanecarboxylic acid;
(1R,3S)-3-(4-(3-(3-chloro-4-isopropoxyphenyl)-1,2,4-oxadiazol-5-yl)pyridin-2-ylamino)cyclopentanecarboxylic acid;
(3S)-3-(4-(3-(3-chloro-4-isopropoxyphenyl)-1,2,4-oxadiazol-5-yl)phenylamino)-1-methylcyclopentanecarboxylic acid;
2-((1R,3S)-3-(4-(3-(3-chloro-4-isopropoxyphenyl)-1,2,4-oxadiazol-5-yl)phenylamino)cyclopentyl)acetic acid;
(1S,3S)-3-(4-(3-(3-chloro-4-isopropoxyphenyl)-1,2,4-oxadiazol-5-yl)phenylamino)-1-methylcyclopentanecarboxylic acid;
(1R,3S)-3-(4-(3-(3-chloro-4-isopropoxyphenyl)-1,2,4-oxadiazol-5-yl)phenylamino)-1-methylcyclopentanecarboxylic acid;
(3S)-3-(4-(3-(3-chloro-4-isopropoxyphenyl)-1,2,4-oxadiazol-5-yl)phenylamino)-1-fluorocyclopentanecarboxylic acid;
(1R,3S)-3-(4-(5-(3-chloro-4-isopropoxyphenyl)-1,2,4-oxadiazol-3-yl)phenylamino)cyclopentanecarboxylic acid;
(3S)-3-(4-(3-(3-chloro-4-isopropoxyphenyl)-1,2,4-oxadiazol-5-yl)phenylamino)-1-hydroxycyclopentanecarboxylic acid;
(1R,3S)-3-(3-chloro-4-(3-(3-chloro-4-isopropoxyphenyl)-1,2,4-oxadiazol-5-yl)phenylamino)cyclopentanecarboxylic acid;
(1R,3S)-3-(4-(3-(3-bromo-4-isopropoxyphenyl)-1,2,4-oxadiazol-5-yl)-3-chlorophenylamino)cyclopentanecarboxylic acid;
(1R,3S)-3-(3-bromo-4-(3-(3-chloro-4-isopropoxyphenyl)-1,2,4-oxadiazol-5-yl)phenylamino)cyclopentanecarboxylic acid;
(1R,3S)-3-(4-(3-(3-chloro-4-isopropoxyphenyl)-1,2,4-oxadiazol-5-yl)-2-(trifluoromethyl)phenylamino)cyclopentanecarboxylic acid;
(R)-3-(4-(3-(3-chloro-4-isopropoxyphenyl)-1,2,4-oxadiazol-5-yl)-1H-indol-1-yl)-2-methylpropanoic acid;
(S)-3-(4-(3-(3-chloro-4-isopropoxyphenyl)-1,2,4-oxadiazol-5-yl)-1H-indol-1-yl)-2-methylpropanoic acid;
(1R,2S)-methyl 2-(4-(3-(3-chloro-4-isopropoxyphenyl)-1,2,4-oxadiazol-5-yl)phenylamino)cyclopentanecarboxylate;
(1S,2R)-methyl 2-(4-(3-(3-chloro-4-isopropoxyphenyl)-1,2,4-oxadiazol-5-yl)phenylamino)cyclopentanecarboxylate;
(1R,2R)-2-(4-(3-(3-chloro-4-isopropoxyphenyl)-1,2,4-oxadiazol-5-yl)phenylamino)cyclopentanecarboxylic acid;
(1S,2S)-2-(4-(3-(3-chloro-4-isopropoxyphenyl)-1,2,4-oxadiazol-5-yl)phenylamino)cyclopentanecarboxylic acid;
3-(3-chloro-4-isopropoxyphenyl)-5-(1-methyl-1,2,3,4-tetrahydroquinolin-6-yl)-1,2,4-oxadiazole;
(R)-3-(4-(3-(4-(tetrahydrofuran-3-yloxy)-3-(trifluoromethyl)phenyl)-1,2,4-oxadiazol-5-yl)-1H-indol-1-yl)propanoic acid;
(1R,3S)-3-[4-(5-biphenyl-2-yl-[1,2,4]oxadiazol-3-yl)-2-methyl-phenylamino]-cyclopentanecarboxylic acid;
(1R,3S)-3-[4-(5-biphenyl-3-yl-[1,2,4]oxadiazol-3-yl)-2-methyl-phenylamino]-cyclopentanecarboxylic acid;
(1R,3S)-3-[4-(5-biphenyl-4-yl-[1,2,4]oxadiazol-3-yl)-2-methyl-phenylamino]-cyclopentanecarboxylic acid;
(1R,3S)-3-{4-[5-(4-cyclohexyl-phenyl)-[1,2,4]oxadiazol-3-yl]-2-methyl-phenylamino}-cyclopentanecarboxylic acid;
(1R,3S)-3-((4-(3-(3-chloro-4-isopropoxyphenyl)-1,2,4-oxadiazol-5-yl)phenyl)(methyl)amino)cyclopentanecarboxylic acid;
methyl 3-(5-(3-(3-chloro-4-isopropoxyphenyl)-1,2,4-oxadiazol-5-yl)-1H-indol-1-yl)cyclopentanecarboxylate;
3-(5-(3-(3-chloro-4-isopropoxyphenyl)-1,2,4-oxadiazol-5-yl)-1H-indol-1-yl)cyclopentanecarboxylic acid;
(1R,3S)-3-(4-(3-(3-chloro-4-isopropoxyphenyl)-1,2,4-oxadiazol-5-yl)-2-ethynylphenylamino)cyclopentanecarboxylic acid;
(1R,3S)-3-(5-(3-(3-chloro-4-isopropoxyphenyl)-1,2,4-oxadiazol-5-yl)-1H-indol-1-yl)cyclopentanecarboxylic acid;
(1S,4R)-2-(4-(3-(3-chloro-4-isopropoxyphenyl)-1,2,4-oxadiazol-5-yl)-3-methoxyphenyl)-2-azabicyclo[2.2.1]heptan-3-one;
(1R,3S)-3-(4-(3-(3-chloro-4-isopropoxyphenyl)-1,2,4-oxadiazol-5-yl)-3-methoxyphenylamino)cyclopentanecarboxylic acid;
(R)-3-(3-(3-(3-chloro-4-isopropoxyphenyl)-1,2,4-oxadiazol-5-yl)-2-methylphenoxy)propane-1,2-diol;
N-(4-(3-(3-chloro-4-isopropoxyphenyl)-1,2,4-oxadiazol-5-yl)phenyl)isonicotinamide;
N-(4-(3-(3-chloro-4-isopropoxyphenyl)-1,2,4-oxadiazol-5-yl)phenyl)isonicotinamide;
(3S)-3-(4-(3-(3-chloro-4-isopropoxyphenyl)-1,2,4-oxadiazol-5-yl)phenylamino)-1-hydroxycyclopentanecarboxylic acid;
(1R,3S)-3-(4-(5-(3-chloro-4-isopropoxyphenyl)-1,2,4-oxadiazol-3-yl)phenylamino)cyclopentanecarboxylic acid;
1-amino-3-(4-(3-(3-chloro-4-isopropoxyphenyl)-1,2,4-oxadiazol-5-yl)phenoxy)cyclopentanecarboxylic acid;
1-amino-3-(3-chloro-4-(5-(5-chloro-6-isopropoxypyridin-3-yl)-1,2,4-oxadiazol-3-yl)phenoxy)cyclopentanecarboxylic acid;
(1R,3S)-3-(4-(5-(3-chloro-4-isopropoxyphenyl)-1,2,4-oxadiazol-3-yl)-3-methylphenylamino)cyclopentanecarboxylic acid;
(1S,4R)-2-(4-(5-(5-chloro-6-isopropoxypyridin-3-yl)-1,2,4-oxadiazol-3-yl)-3-methylphenyl)-2-azabicyclo[2.2.1]heptan-3-one;
(1R,3S)-3-(4-(5-(5-chloro-6-isopropoxypyridin-3-yl)-1,2,4-oxadiazol-3-yl)-3-methylphenylamino)cyclopentanecarboxylic acid;
methyl 2-(5-(3-(3-(3-chloro-4-isopropoxyphenyl)-1,2,4-oxadiazol-5-yl)phenyl)-2H-tetrazol-2-yl)acetate;
3-(3-(3-(3-chloro-4-isopropoxyphenyl)-1,2,4-oxadiazol-5-yl)phenyl)-1,2,4-oxadiazol-5(2H)-one;
(1R,3S)-3-(4-(3-(3-chloro-4-isopropoxyphenyl)-1,2,4-oxadiazol-5-yl)-3-ethylphenylamino)cyclopentanecarboxylic acid;
(1R,3S)-3-(4-(3-(3-chloro-4-isopropoxyphenyl)-1,2,4-oxadiazol-5-yl)-3-cyanophenylamino)cyclopentanecarboxylic acid;
(2R,4R)-4-(4-(3-(3-chloro-4-isopropoxyphenyl)-1,2,4-oxadiazol-5-yl)phenylamino)pyrrolidine-2-carboxylic acid;
2-(4-(3-(4-isopropoxy-3-(trifluoromethyl)phenyl)-1,2,4-oxadiazol-5-yl)phenylamino)propan-1-ol;
(R)-2-(4-(3-(4-isopropoxy-3-(trifluoromethyl)phenyl)-1,2,4-oxadiazol-5-yl)phenoxy)propanoic acid;
(R)—N-(2-hydroxyethyl)-2-(4-(3-(4-isopropoxy-3-(trifluoromethyl)phenyl)-1,2,4-oxadiazol-5-yl)phenoxy)propanamide;
(R)-2-(4-(3-(4-isopropoxy-3-(trifluoromethyl)phenyl)-1,2,4-oxadiazol-5-yl)phenoxy)propanal;
3-{3-Chloro-4-[5-(5-chloro-6-isopropoxy-pyridin-3-yl)-[1,2,4]oxadiazol-3-yl]-phenoxy}-cyclobutanecarboxylic acid; or
(1R,3S)-3-(4-(5-(5-chloro-6-isopropoxypyridin-3-yl)-1,2,4-oxadiazol-3-yl)phenylamino)cyclopentane carboxylic acid.

In a tenth embodiment the invention provides a compound of Formula (II)

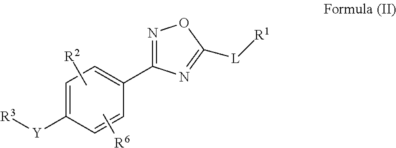

pharmaceutically acceptable salts, biologically active metabolites, solvates, hydrates, prodrugs, enantiomers or stereoisomers thereof, wherein

Y is a bond;

L is a bond or CH₂;

R¹ is optionally substituted (C₁-C₄)alkyl, optionally substituted indolyl or optionally substituted phenyl;

R² is CF₃,

R³ is H, morpholinyl, optionally substituted piperidine or (C₃-C₅)cycloalkyl; and

R⁶ is H.

In an eleventh embodiment the invention provides a compound according to the tenth embodiment wherein R¹ is optionally substituted by one or more substituents independently selected from Cl, F, CN, optionally substituted (C₁-C₃)alkyl, —CH₂-optionally substituted azetidinyl, —CH₂-optionally substituted pyrrolidinyl, —CH₂NR^{c}R^{d}, —NH-optionally substituted (C₃-C₆)cycloalkyl, optionally substituted piperidinyl,

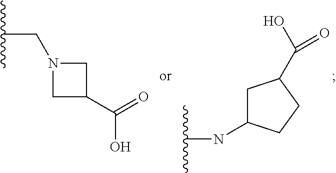

wherein R^{c} and R^{d} are independently H, optionally substituted (C₁-C₆)alkyl or optionally substituted (C₃-C₆)cycloalkyl;

In a twelfth embodiment the invention provides a compound according to the tenth and eleventh embodiments wherein the compound is
1-((4-(3-(4-(4-fluoropiperidin-1-yl)-3-(trifluoromethyl)phenyl)-1,2,4-oxadiazol-5-yl)benzylamino)methyl)cyclopropanecarboxylic acid;
(R)-1-(4-(3-(4-(4-fluoropiperidin-1-yl)-3-(trifluoromethyl)phenyl)-1,2,4-oxadiazol-5-yl)benzyl)pyrrolidine-3-carboxylic acid;
(S)-1-(4-(3-(4-(4-fluoropiperidin-1-yl)-3-(trifluoromethyl)phenyl)-1,2,4-oxadiazol-5-yl)benzyl)pyrrolidine-3-carboxylic acid;
1-(4-(3-(4-(4-fluoropiperidin-1-yl)-3-(trifluoromethyl)phenyl)-1,2,4-oxadiazol-5-yl)benzylamino)cyclopropanecarboxylic acid;
1-(4-{3-[4-(4-fluoro-piperidin-1-yl)-3-trifluoromethyl-phenyl]-[1,2,4]oxadiazol-5-yl}-benzylamino)-cyclopropanecarboxylic acid;
1-(4-{3-[4-(4-fluoro-piperidin-1-yl)-3-trifluoromethyl-phenyl]-[1,2,4]oxadiazol-5-yl}-benzyl)-pyrrolidine-3-carboxylic acid;
1-(4-{3-[4-(4-fluoro-piperidin-1-yl)-3-trifluoromethyl-phenyl]-[1,2,4]oxadiazol-5-yl}-benzyl)-4-methyl-pyrrolidine-3-carboxylic acid;
4-fluoro-piperidin-1-yl)-3-trifluoromethyl-phenyl]-[1,2,4]oxadiazol-5-yl}-benzylamino)-acetic acid;
[(S)-1-(4-{3-[4-(4-fluoro-piperidin-1-yl)-3-trifluoromethyl-phenyl]-[1,2,4]oxadiazol-5-yl}-benzyl)-pyrrolidin-2-yl]-acetic acid;
[1-(4-{3-[4-(4-fluoro-piperidin-1-yl)-3-trifluoromethyl-phenyl]-[1,2,4]oxadiazol-5-yl}-benzylamino)-cyclopropyl]-methanol;
1-(4-{3-[4-(4-fluoro-piperidin-1-yl)-3-trifluoromethyl-phenyl]-[1,2,4]oxadiazol-5-yl}-benzyl)-4,4-dimethyl-pyrrolidine-3-carboxylic acid;
1-[(4-{3-[4-(4-fluoro-piperidin-1-yl)-3-trifluoromethyl-phenyl]-[1,2,4]oxadiazol-5-yl}-benzylamino)-methyl]-cyclopropanol;
(1R,3S)-3-(4-(3-(3-chloro-4-isopropoxyphenyl)-1,2,4-oxadiazol-5-yl)-3-(trifluoromethyl)phenylamino)cyclopentanecarboxylic acid;
(1R,3S)-3-(4-(3-(3-chloro-4-isopropoxyphenyl)-1,2,4-oxadiazol-5-yl)-3-methylphenylamino)cyclopentanecarboxylic acid;
(1R,3S)-3-(4-(3-(3-chloro-4-isopropoxyphenyl)-1,2,4-oxadiazol-5-yl)-2-methylphenylamino)cyclopentanecarboxylic acid;
4-(4-(3-(3-chloro-4-isopropoxyphenyl)-1,2,4-oxadiazol-5-yl)phenylamino)-2-methylbutanoic acid;
tert-butyl 2-(4-(3-(3-chloro-4-isopropoxyphenyl)-1,2,4-oxadiazol-5-yl)phenylamino)acetate;
tert-butyl 3-(4-(3-(3-chloro-4-isopropoxyphenyl)-1,2,4-oxadiazol-5-yl)phenylamino)propanoate;
1-amino-3-(4-(3-(3-chloro-4-isopropoxyphenyl)-1,2,4-oxadiazol-5-yl)phenoxy)cyclopentanecarboxylic acid;
3-{4-[3-(3-chloro-4-isopropoxy-phenyl)-[1,2,4]oxadiazol-5-yl]-phenoxy}-cyclobutanecarboxylic acid;
2-(6-(3-(3-chloro-4-isopropoxyphenyl)-1,2,4-oxadiazol-5-yl)-3,4-dihydroquinolin-1(2H)-yl)acetic acid;
3-(6-(3-(3-chloro-4-isopropoxyphenyl)-1,2,4-oxadiazol-5-yl)-3,4-dihydroquinolin-1(2H)-yl)propanoic acid;
(E)-4-(3-(3-(3-chloro-4-isopropoxyphenyl)-1,2,4-oxadiazol-5-yl)-2-methylphenoxy)but-2-enoic acid;
4-(3-(3-(3-chloro-4-isopropoxyphenyl)-1,2,4-oxadiazol-5-yl)-2-methylphenoxy)butanoic acid;
4-(3-(3-(3-chloro-4-isopropoxyphenyl)-1,2,4-oxadiazol-5-yl)-4-methylphenoxy)butanoic acid;
(3-{4-[3-(3-chloro-4-isopropoxy-phenyl)-[1,2,4]oxadiazol-5-yl]-phenylamino}-propyl)-phosphonic acid diethyl ester;
(3-{4-[3-(3-chloro-4-isopropoxy-phenyl)-[1,2,4]oxadiazol-5-yl]-benzylamino}-propyl)-phosphonic acid;
(1S,3R)-3-(4-(3-(3-chloro-4-isopropoxyphenyl)-1,2,4-oxadiazol-5-yl)phenylamino)cyclopentylphosphonic acid;
(1R,3R)-3-(4-(3-(3-chloro-4-isopropoxyphenyl)-1,2,4-oxadiazol-5-yl)phenylamino)cyclopentylphosphonic acid;
(1R,3R)-3-(2-bromo-4-(3-(3-chloro-4-isopropoxyphenyl)-1,2,4-oxadiazol-5-yl)phenylamino)cyclopentylphosphonic acid;
(1R,3S)-3-(4-(3-(3-chloro-4-isopropoxyphenyl)-1,2,4-oxadiazol-5-yl)phenylamino)cyclopentylphosphonic acid;
(1R,3S)-3-(2-bromo-4-(3-(3-chloro-4-isopropoxyphenyl)-1,2,4-oxadiazol-5-yl)phenylamino)cyclopentylphosphonic acid;
(1S,3S)-3-(4-(3-(3-chloro-4-isopropoxyphenyl)-1,2,4-oxadiazol-5-yl)phenylamino)cyclopentylphosphonic acid;
3-(4-(3-(3-chloro-4-isopropoxyphenyl)-1,2,4-oxadiazol-5-yl)benzyl)cyclobutanecarboxylic acid;
3-(4-(3-(3-chloro-4-isopropoxyphenyl)-1,2,4-oxadiazol-5-yl)benzyl)cyclopentanecarboxylic acid;
1-(4-(3-(3-chloro-4-isopropoxyphenyl)-1,2,4-oxadiazol-5-yl)benzyl)azetidine-3-carboxylic acid;
2-(4-(3-(3-chloro-4-isopropoxyphenyl)-1,2,4-oxadiazol-5-yl)phenyl)propan-2-amine;
methyl 3-(2-(4-(3-(3-chloro-4-isopropoxyphenyl)-1,2,4-oxadiazol-5-yl)phenyl)propan-2-ylamino)propanoate;
3-(2-(4-(3-(3-chloro-4-isopropoxyphenyl)-1,2,4-oxadiazol-5-yl)phenyl)propan-2-ylamino)propanoic acid;
3-(3-chloro-4-isopropoxyphenyl)-5-(1H-indol-4-yl)-1,2,4-oxadiazole;
(4-(3-(3-chloro-4-isopropoxyphenyl)-1,2,4-oxadiazol-5-yl)phenyl)methanamine;
3-(3-(3-(3-chloro-4-isopropoxyphenyl)-1,2,4-oxadiazol-5-yl)cyclopentylamino)propanoic acid;
4-(3-(3-(3-chloro-4-isopropoxyphenyl)-1,2,4-oxadiazol-5-yl)cyclopentylamino)butanoic acid;
(S)-3-(4-(3-(3-chloro-4-isopropoxyphenyl)-1,2,4-oxadiazol-5-yl)phenoxy)propane-1,2-diol;
4-(3-(3-chloro-4-isopropoxyphenyl)-1,2,4-oxadiazol-5-yl)benzenesulfonamide;
tert-butyl 3,3′-(4-(3-(3-chloro-4-isopropoxyphenyl)-1,2,4-oxadiazol-5-yl)phenylsulfonylazanediyl)dipropanoate;
tert-butyl 3-(4-(3-(3-chloro-4-isopropoxyphenyl)-1,2,4-oxadiazol-5-yl)phenylsulfonamido)propanoate;
3-(4-(3-(3-chloro-4-isopropoxyphenyl)-1,2,4-oxadiazol-5-yl)phenylsulfonamido)propanoic acid;
2,2′-(4-(3-(3-chloro-4-isopropoxyphenyl)-1,2,4-oxadiazol-5-yl)phenylsulfonylazanediyl)diacetic acid;
tert-butyl 2,2′-(4-(3-(3-chloro-4-isopropoxyphenyl)-1,2,4-oxadiazol-5-yl)phenylsulfonylazanediyl)diacetate;
tert-butyl 2-(4-(3-(3-chloro-4-isopropoxyphenyl)-1,2,4-oxadiazol-5-yl)phenylsulfonamido)acetate;
2-(4-(3-(3-chloro-4-isopropoxyphenyl)-1,2,4-oxadiazol-5-yl)phenylsulfonamido)acetic acid;
tert-butyl 2-(5-(3-(3-chloro-4-isopropoxyphenyl)-1,2,4-oxadiazol-5-yl)-3,4-dihydroisoquinolin-2(1H)-yl)acetate;
tert-butyl 5-(3-(3-chloro-4-isopropoxyphenyl)-1,2,4-oxadiazol-5-yl)-3,4-dihydroisoquinoline-2(1H)-carboxylate;
3-(3-chloro-4-isopropoxyphenyl)-5-(1,2,3,4-tetrahydroisoquinolin-5-yl)-1,2,4-oxadiazole;
2-(5-(3-(3-chloro-4-isopropoxyphenyl)-1,2,4-oxadiazol-5-yl)-3,4-dihydroisoquinolin-2(1H)-yl)acetic acid;
tert-butyl 3-(5-(3-(3-chloro-4-isopropoxyphenyl)-1,2,4-oxadiazol-5-yl)-3,4-dihydroisoquinolin-2(1H)-yl)propanoate;
3-(5-(3-(3-chloro-4-isopropoxyphenyl)-1,2,4-oxadiazol-5-yl)-3,4-dihydroisoquinolin-2(1H)-yl)propanoic acid;
2-[3-(3-chloro-4-isopropoxy-phenyl)-[1,2,4]oxadiazol-5-yl]-3-methyl-5,6,7,8-tetrahydro-imidazo[1,2-a]pyrazine;
1-{2-[3-(3-chloro-4-isopropoxy-phenyl)-[1,2,4]oxadiazol-5-yl]-3-methyl-5,6-dihydro-8H-imidazo[1,2-a]pyrazin-7-yl}-ethanone;
{2-[3-(3-chloro-4-isopropoxy-phenyl)-[1,2,4]oxadiazol-5-yl]-3-methyl-5,6-dihydro-8H-imidazo[1,2-a]pyrazin-7-yl}-acetic acid tert-butyl ester;
{2-[3-(3-chloro-4-isopropoxy-phenyl)-[1,2,4]oxadiazol-5-yl]-3-methyl-5,6-dihydro-8H-imidazo[1,2-a]pyrazin-7-yl}-acetic acid;
3-[3-(3-chloro-4-isopropoxy-phenyl)-[1,2,4]oxadiazol-5-yl]-2-methyl-imidazo[1,2-a]pyrazine;
3-(3-chloro-4-isopropoxyphenyl)-5-(4-((2,2-dimethyl-1,3-dioxolan-4-yl)methoxy)phenyl)-1,2,4-oxadiazole;
2-(4-(3-(3-chloro-4-isopropoxyphenyl)-1,2,4-oxadiazol-5-yl)phenoxy)acetic acid;
1-(4-(3-(3-cyano-4-isopropoxyphenyl)-1,2,4-oxadiazol-5-yl)benzyl)azetidine-3-carboxylic acid;
1-(4-(3-(3-chloro-4-isopropoxyphenyl)-1,2,4-oxadiazol-5-yl)phenyl)cyclopropanecarbonitrile;
1-(4-(3-(3-chloro-4-isopropoxyphenyl)-1,2,4-oxadiazol-5-yl)phenyl)cyclopropanecarbaldehyde;
3-((1-(4-(3-(3-chloro-4-isopropoxyphenyl)-1,2,4-oxadiazol-5-yl)phenyl)cyclopropyl)methylamino)propanoic acid;
N-(4-(3-(3-chloro-4-isopropoxyphenyl)-1,2,4-oxadiazol-5-yl)benzyl)-1-(2,2-dimethyl-1,3-dioxolan-4-yl)methanamine;
3-(4-(3-(3-chloro-4-isopropoxyphenyl)-1,2,4-oxadiazol-5-yl)benzylamino)propane-1,2-diol;
(Z)-methyl 3-(4-(3-(3-chloro-4-isopropoxyphenyl)-1,2,4-oxadiazol-5-yl)phenyl)acrylate;
trans-methyl 2-(4-(3-(3-chloro-4-isopropoxyphenyl)-1,2,4-oxadiazol-5-yl)phenyl)cyclopropanecarboxylate;
trans-2-(4-(3-(3-chloro-4-isopropoxyphenyl)-1,2,4-oxadiazol-5-yl)phenyl)cyclopropanecarboxylic acid;
tert-butyl 5-(3-(3-chloro-4-isopropoxyphenyl)-1,2,4-oxadiazol-5-yl)isoindoline-2-carboxylate;
3-(3-chloro-4-isopropoxyphenyl)-5-(isoindolin-5-yl)-1,2,4-oxadiazole;
methyl 3-(5-(3-(3-chloro-4-isopropoxyphenyl)-1,2,4-oxadiazol-5-yl)isoindolin-2-yl)propanoate;
3-(5-(3-(3-chloro-4-isopropoxyphenyl)-1,2,4-oxadiazol-5-yl)isoindolin-2-yl)propanoic acid;
(Z)-methyl 3-(4-(3-(3-chloro-4-isopropoxyphenyl)-1,2,4-oxadiazol-5-yl)phenyl)acrylate;
(Z)-3-(4-(3-(3-chloro-4-isopropoxyphenyl)-1,2,4-oxadiazol-5-yl)phenyl)acrylic acid;
3-(3-chloro-4-(3-(3-chloro-4-isopropoxyphenyl)-1,2,4-oxadiazol-5-yl)phenylamino)cyclobutanecarboxylic acid;
3-(4-(3-(3-chloro-4-isopropoxyphenyl)-1,2,4-oxadiazol-5-yl)phenylamino)cyclobutanecarboxylic acid;
1-(4-(3-(4-(4-fluoropiperidin-1-yl)-3-(trifluoromethyl)phenyl)-1,2,4-oxadiazol-5-yl)benzyl)azetidine-3-carboxylic acid;
of 5-((4-(3-(4-(4-fluoropiperidin-1-yl)-3-(trifluoromethyl)phenyl)-1,2,4-oxadiazol-5-yl)benzylamino)methyl)isoxazol-3-ol;
2-((4-(3-(4-isopropoxy-3-(trifluoromethyl)phenyl)-1,2,4-oxadiazol-5-yl)phenoxy)methyl)morpholine;
(1R,3S)-3-(4-(3-(4-((S)-tetrahydrofuran-3-yloxy)-3-(trifluoromethyl)phenyl)-1,2,4-oxadiazol-5-yl)phenylamino)cyclopentanecarboxylic acid;
(1R,3S)-3-(4-(3-(4-(4-fluoropiperidin-1-yl)-3-(trifluoromethyl)phenyl)-1,2,4-oxadiazol-5-yl)phenylamino)cyclopentanecarboxylic acid;
(1R,3S)-3-(4-(3-(4-(4,4-difluoropiperidin-1-yl)-3-(trifluoromethyl)phenyl)-1,2,4-oxadiazol-5-yl)phenylamino)cyclopentanecarboxylic acid;
(1S,3S)-3-(4-(3-(4-(4-fluoropiperidin-1-yl)-3-(trifluoromethyl)phenyl)-1,2,4-oxadiazol-5-yl)phenylamino)cyclopentanecarboxylic acid;
(1R,3R)-3-(4-(3-(4-(4-fluoropiperidin-1-yl)-3-(trifluoromethyl)phenyl)-1,2,4-oxadiazol-5-yl)phenylamino)cyclopentanecarboxylic acid;
(1S,3R)-3-(4-(5-(3-cyano-4-(4-fluoropiperidin-1-yl)phenyl)-1,2,4-oxadiazol-3-yl)-2-(trifluoromethyl)phenylamino)cyclopentanecarboxylic acid;
(1S,3R)-3-(4-(5-(3-cyano-4-(4-fluoropiperidin-1-yl)phenyl)-1,2,4-oxadiazol-3-yl)phenylamino)cyclopentanecarboxylic acid;
5-[3-(4-Fluoro-phenyl)-[1,2,4]oxadiazol-5-yl]-2-(4-fluoro-piperidin-1-yl)-benzonitrile;
5-[3-(4-Fluoro-3-trifluoromethyl-phenyl)-[1,2,4]oxadiazol-5-yl]-2-(4-fluoro-piperidin-1-yl)-benzonitrile;
(1R,3S)-3-[4-(5-biphenyl-2-yl-[1,2,4]oxadiazol-3-yl)-2-methyl-phenylamino]-cyclopentanecarboxylic acid;
(1R,3S)-3-[4-(5-biphenyl-3-yl-[1,2,4]oxadiazol-3-yl)-2-methyl-phenylamino]-cyclopentanecarboxylic acid;
(1R,3S)-3-{4-[5-(4-cyclohexyl-phenyl)-[1,2,4]oxadiazol-3-yl]-2-methyl-phenylamino}-cyclopentanecarboxylic acid; or
(1R,3S)-3-(4-(5-(4-isobutylphenyl)-1,2,4-oxadiazol-3-yl)phenylamino)cyclopentanecarboxylic acid.

In a thirteenth embodiment the invention provides a compound of Formula (III)

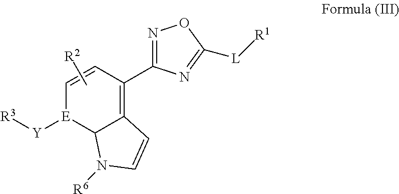

pharmaceutically acceptable salts, biologically active metabolites, solvates, hydrates, prodrugs, enantiomers or stereoisomers thereof, wherein

E is CH or N;

Y is a bond;

L is a bond;

R¹ is optionally substituted aryl;

R² is H;

R³ is H; and

R⁶ is H or optionally substituted (C₁-C₃)alkyl.

N a fourteenth embodiment the invention provides a compound of Formula (IV):

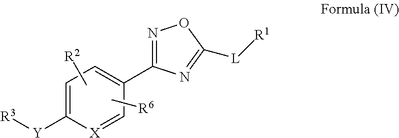

or a pharmaceutically acceptable salt, solvate, hydrate, metabolite, prodrug, enantiomer or stereoisomer thereof,

wherein:

X is N or CR⁴;

L is a bond, —CH₂CH₂—, (C₃-C₆)cycloalkyl, or —CHR⁵;

Y is —O—, —NR⁷— or —C(R⁷)(R^{7′})—;

R¹ is optionally substituted aryl, optionally substituted heteroaryl, optionally substituted heterocyclyl, optionally substituted —(C₁-C₆)alkyl-O—(C₁-C₃)alkyl, optionally substituted —(C₁-C₆)alkyl-O—(C₁-C₆)alkyl-O—(C₁-C₃)alkyl, optionally substituted —(C₁-C₆)alkyl-O-aryl, alkylsulfanylalkyl, unsubstituted (C₂-C₅)alkyl, substituted (C₁-C₆)alkyl, —COR¹¹, optionally substituted —O—(C₁-C₃)alkyl, —N(R⁷)(R⁸), —N(R⁷)SO₂—R¹¹ or optionally substituted (C₃-C₆)cycloalkyl, and wherein R¹ is not substituted cyclopentathiophene, halothiophene, substituted indan or substituted chromenone;

R² and R⁶ may be the same or different and are independently H, —(C₁-C₄)alkyl, —O—(C₁-C₃)alkyl, —CF₃, —CN, halo or —COO—(C₁-C₄)alkyl;

R³ is optionally substituted aryl, optionally substituted heterocyclyl, optionally substituted heteroaryl, optionally substituted (C₃-C₆)cycloalkyl, —(CH₂)ₙ—R¹¹, —CO—OR¹¹, —CO—R¹¹, —CON(R⁷)(R¹¹), —N(R⁷)(R¹¹), —SOR¹¹, —SO₂R¹¹ and optionally substituted straight or branched (C₁-C₈)alkyl chain optionally including —CO—, —COO—, —SO—, —SO₂—, —CONH—, —NHCO—, —N— or —O— groups embedded within the alkyl chain; and when Y is O, R³ is not alkyldiazeapane, —C(CH₃)₂COOCH₂CH₃ or —CH₂CH₂N(CH₂CH₃)₂, and when Y is —CH₂—, R³ is not —CH₂COOH;

or Y is a bond and R³ is optionally substituted morpholino;

R⁴ is H, —(C₁-C₄)alkyl, —O—(C₁-C₃)alkyl, —CF₃, —CN or halo;

R⁵ is H, O—(C₁-C₃)alkyl or (C₁-C₃)alkyl;

each occurrence of R⁷ or R^{7′} is independently H or optionally substituted (C₁-C₃)alkyl;

R⁸ is H, optionally substituted CH₃, or —COR¹¹;

R¹¹ is hydrogen, optionally substituted (C₁-C₃)alkyl, optionally substituted alkynyl, optionally substituted aryl, optionally substituted heteroaryl, optionally substituted heterocyclyl or optionally substituted (C₃-C₆)cycloalkyl; and

n is 1, 2, 3 or 4;

provided that

R¹ is not optionally substituted furanyl or —C(O)-optionally substituted furanyl;

R³ is not optionally substituted quinolinyl;

R¹¹ is not optionally substituted cyclopropyl, optionally substituted cyclohexyl, optionally substituted furanyl, optionally substituted imidazolyl, optionally substituted indolyl, optionally substituted naphthyl, optionally substituted piperazinyl, optionally substituted pyrazolyl, optionally substituted pyridazinyl or optionally substituted quinolinyl;

R¹ is not substituted by —C(O)-cyclopentyl, optionally substituted cyclopentyl, —C(O)-cyclobutyl, cyclobutyl, —C(O)-cyclohexyl or optionally substituted cyclohexyl;

R³ is not substituted by —C(O)-cyclopropyl;

when R³ is CH₃ or 4-chlorophenylmethyl, L-R¹ is not cyclopropyl, cyclopentyl, optionally substituted cyclohexyl, —CH₂-cyclohexyl, —NH-cyclohexyl, —CH₂CH₂-cyclohexyl or optionally substituted pyrazolyl;

when Y is O, R³ is not —(C₀-C₄)alkyl-optionally substituted isoxazolyl or optionally substituted pyrazolyl;

when L is (C₁-C₃)alkyl, R¹ is not optionally substituted isoxazolyl;

when L is a bond, R¹ is not optionally substituted cyclobutyl, optionally substituted cyclohexyl, optionally substituted naphthyl, —CH₂-optionally substituted naphthyl, —CH₂—O-optionally substituted naphthyl, optionally substituted pyrazolyl or tetrahydrobenzofuranyl;

provided the compound is not

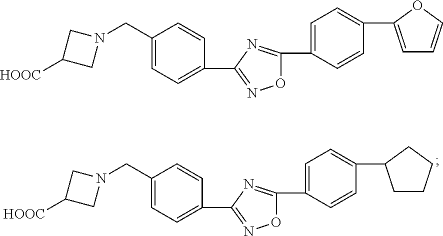

provided the compound is not

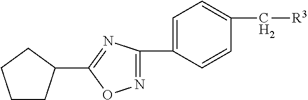

wherein R³ is optionally substituted piperazinyl or optionally substituted phenyl;

provided the compound is not

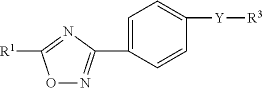

wherein R¹ is optionally substituted pyridine or 3-chlorophenyl and —Y—R³ is

—NH—C(O)-optionally substituted phenyl;

—O-optionally substituted pyridinyl;

—NH—C(O)—OCH₃;

—CH₂-optionally substituted piperazinyl;

—O-optionally substituted (C₁-C₉)alkyl;

—CH₂-morpholinyl; or

—O—C(O)-optionally substituted pyridinyl;

provided the compound is not

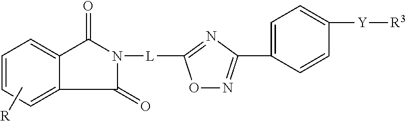

wherein

L is CH₂, CH(CH₃) or CH₂CH₂;

Y is O or CH₂;

R² is H or OCH₃;

R³ is CH₃ or OCF₃; and

R is H or NO₂;

provided the compound is not

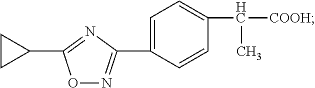

provided the compound is not

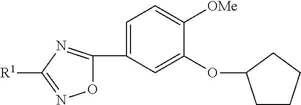

wherein R¹ is phenyl, 4-chlorophenyl, piperidinyl or thienyl.

In a fifteenth embodiment the invention provides a compound according t the fourteenth embodiment wherein

R¹ is optionally substituted phenyl, optionally substituted tetrahydrobenzofuranyl, optionally substituted furanyl, optionally substituted 2,3-dihydroisoindolyl, optionally substituted isoindolinyl, optionally substituted imidazolyl, optionally substituted 5,6-dihydro imidazo[1,2-a]pyrazinyl, optionally substituted imidazo[1,2-a]pyrazinyl, optionally substituted indolyl, optionally substituted isoxazolyl, optionally substituted pyrazolyl, optionally substituted pyridinyl, optionally substituted pyrimidinyl, optionally substituted pyrrolidinyl, optionally substituted 1,2,3,4-tetrahydroisoquinolinyl, optionally substituted quinolinyl, optionally substituted 3,4-dihydroquinolinyl, optionally substituted 3,4-dihydroisoquinolinyl, optionally substituted 5,6,7,8-tetrahydroimidazo[1,2-a]pyrazinyl, optionally substituted pyrrolyl, optionally substituted pyrrolo[2,3-b]pyridinyl, optionally substituted quinolinyl, optionally substituted thiazolyl, optionally substituted thienyl, optionally substituted —(C₁-C₆)alkyl-O—(C₁-C₃)alkyl, optionally substituted —(C₁-C₆)alkyl-O—(C₁-C₆)alkyl-O—(C₁-C₃)alkyl, optionally substituted —(C₁-C₆)alkyl-O-phenyl, unsubstituted (C₂-C₅)alkyl, substituted (C₁-C₆)alkyl, —COR¹¹, optionally substituted —O—(C₁-C₃)alkyl, —N(R⁷)(R⁸), —N(R⁷)SO₂—R¹¹ or optionally substituted (C₃-C₆)cycloalkyl;

R² and R⁶ may be the same or different and are independently H, —(C₁-C₄)alkyl, —O—(C₁-C₃)alkyl, —CF₃, —CN, Cl, or F.

In a sixteenth embodiment the invention provides a compound according to the fourteenth and fifteenth embodiments wherein

L is a bond, —CH₂CH₂—, or —CHR⁵;

Y is —O—, —NR⁷— or —C(R⁷)(R^{7′})—;

R¹ is optionally substituted phenyl, optionally substituted furanyl, optionally substituted isoindolinyl, optionally substituted imidazolyl, optionally substituted imidazo[1,2-a]pyrazinyl, optionally substituted indolyl, optionally substituted isoxazolyl, optionally substituted pyrazolyl, optionally substituted pyridinyl, optionally substituted pyrimidinyl, optionally substituted pyrrolidinyl, optionally substituted quinolinyl, optionally substituted pyrrolyl, optionally substituted pyrrolo[2,3-b]pyridinyl, optionally substituted quinolinyl, optionally substituted thiazolyl, optionally substituted thienyl, optionally substituted —(C₁-C₆)alkyl-O—(C₁-C₃)alkyl, optionally substituted —(C₁-C₆)alkyl-O-phenyl, unsubstituted (C₂-C₅)alkyl, substituted (C₁-C₆)alkyl, —COR¹¹, optionally substituted —O—(C₁-C₃)alkyl, —N(R⁷)(R⁸), —N(R⁷)SO₂—R¹¹ or optionally substituted (C₃-C₆)cycloalkyl;

R² and R⁶ may be the same or different and are independently H, —(C₁-C₄)alkyl, —O—(C₁-C₃)alkyl, —CF₃, —CN, Cl or F;

R³ is optionally substituted phenyl, optionally substituted piperidinyl, optionally substituted furanyl, optionally substituted pyrimidinyl, optionally substituted pyridinyl, optionally substituted (C₃-C₆)cycloalkyl, —(CH₂)ₙ—R¹¹, —CO—OR¹¹, —CO—R¹¹, —CON(R⁷)(R¹¹), —N(R⁷)(R¹¹), —SOR¹¹, —SO₂R¹¹ and optionally substituted straight or branched (C₁-C₈)alkyl chain.

In a seventeenth embodiment the invention provides a compound according to the fourteenth through sixteenth embodiments wherein R¹ is optionally substituted phenyl, optionally substituted furanyl, optionally substituted indolyl, optionally substituted isoxazolyl, optionally substituted pyrazolyl, optionally substituted pyridinyl, optionally substituted pyrimidinyl, optionally substituted pyrrolyl, unsubstituted (C₂-C₅)alkyl, substituted (C₁-C₆)alkyl, —COR¹¹, —N(R⁷)(R⁸), optionally substituted —O—(C₁-C₃)alkyl, or optionally substituted (C₃-C₆)cycloalkyl;

R² and R⁶ may be the same or different and are independently H, —(C₁-C₄)alkyl, —CF₃, Cl or F;

R³ is optionally substituted phenyl, optionally substituted piperidinyl optionally substituted pyrimidinyl, optionally substituted pyridinyl, optionally substituted (C₃-C₆)cycloalkyl, —(CH₂)ₙ—R¹¹, optionally substituted straight or branched (C₁-C₈)alkyl chain or

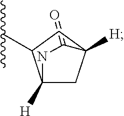

or

—Y—R³ is

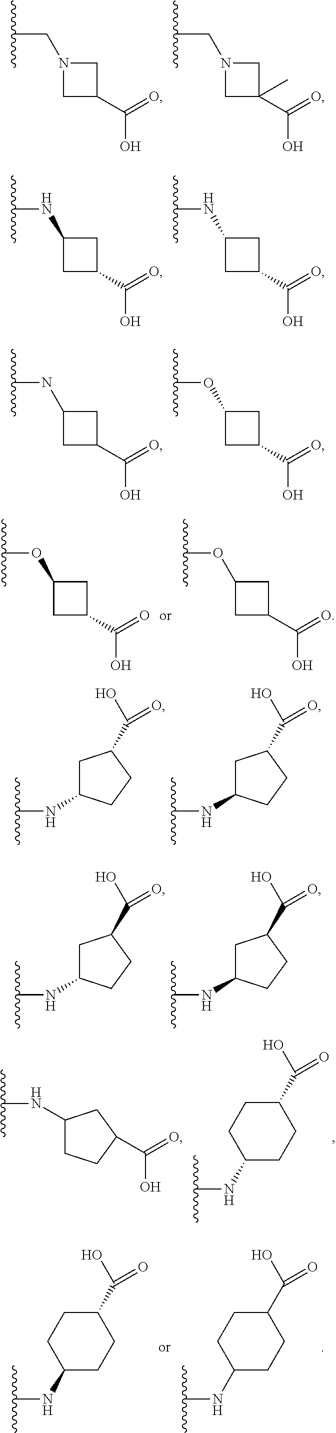

In an eighteenth embodiment the invention provides a compound according to the fourteenth through seventeenth embodiments wherein R¹ is optionally substituted by one or more substituents independently selected from Br, Cl, F, CF₃, CN, oxo, —C(═O)H, —N(R⁹)₂, optionally substituted (C₁-C₆)alkyl, optionally substituted (C₂-C₆)alkenyl, optionally substituted (C₂-C₆)alkynyl, optionally substituted azabicyclo[2.2.1]heptanyl, optionally substituted (C₃-C₆)cycloalkyl, —C(R⁹)₂-optionally substituted (C₃-C₆)cycloalkyl, —C(R⁹)₂-optionally substituted azetidinyl, —CR⁹₂-optionally substituted piperidinyl, —C(R⁹)₂-optionally substituted pyrrolidinyl, —C(R⁹)₂—N(R⁹)₂, —C(O)-optionally substituted (C₁-C₆)alkyl, —C(O)—NR⁹—(C₁-C₆)alkyl, —C(O)—O-optionally substituted (C₁-C₆)alkyl, —C(R⁹)₂—C(O)—O-optionally substituted (C₁-C₆)alkyl, —NR⁹-optionally substituted (C₃-C₆)cycloalkyl, —NR⁹-optionally substituted azetidinyl, —NR₉-furanyl, —NR⁹-optionally substituted pyrrolidinyl, —NR⁹—C(O)—O-optionally substituted (C₁-C₃)alkyl, —NR⁹-optionally substituted (C₁-C₆)alkyl, —NR⁹-optionally substituted (C₃-C₆)cycloalkyl, —NR⁹—C(O)-azetidinyl, —NR⁹—C(O)-furanyl, —NR⁹—C(O)-pyridinyl, —NR⁹—C(O)-optionally substituted pyrrolidinyl, —NR⁹—S(O)₂-optionally substituted phenyl, —O-optionally substituted (C₁-C₆)alkyl, —O-deuterated —(C₂-C₆)alkyl, —O-optionally substituted (C₂-C₆)alkenyl, —O-optionally substituted (C₃-C₆)cycloalkyl, —O-1H-benzo[d][1,2,3]triazolyl, —S(O)₂—N(R⁹)₂, —S(O)₂—NR⁹-optionally substituted (C₁-C₄)alkyl, optionally substituted azetidinyl, optionally substituted piperidinyl, optionally substituted pyridinyl, optionally substituted pyrrolidinyl, optionally substituted 1,2,4 oxadiazolyl, optionally substituted pyrrolidinyl, optionally substituted tetrazolyl,

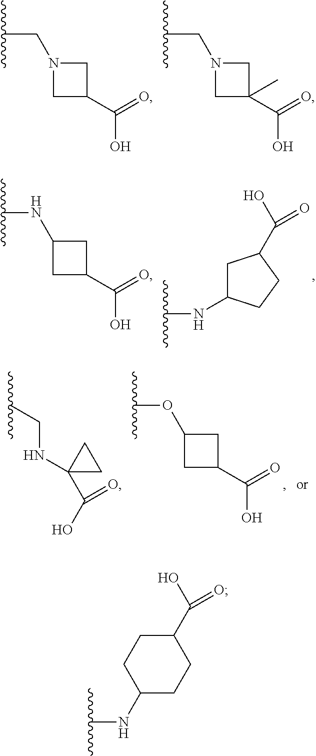

and

wherein each R⁹ is independently selected from H or optionally substituted (C₁-C₆)alkyl.

In a nineteenth embodiment the invention provides a compound according to the fourteenth through eighteenth embodiments wherein each substituent or optional substituent is independently one or more R¹⁰ groups wherein R¹⁰ is optionally substituted alkyl, alkenyl, optionally substituted alkoxy groups, alkoxyalkoxy, alkoxyalkyl, alkoxycarbonyl, alkoxycarbonylheterocycloalkoxy, alkyl, alkylamino, alkylcarbonyl, alkylester, alkyl-O—C(O)—, alkyl-heterocyclyl, alkyl-cycloalkyl, alkyl-nitrile, alkylsulfonyl, alkynyl, amido groups, optionally substituted amino, aminoalkyl, aminoalkoxy, aminocarbonyl, optionally substituted azabicyclo[2.2.1]heptanyl, carbonitrile, carbonylalkoxy, carboxamido, CF₃, CN, —C(O)OH, —C(O)H, —C(O)—C(CH₃)₃, —OH, —C(O)O-alkyl, —C(O)O-optionally substituted cycloalkyl, —C(O)O-heterocyclyl, —C(O)-alkyl, —C(O)— optionally substituted cycloalkyl, —C(O)-heterocyclyl, CN, optionally substituted cycloalkyl, dialkylamino, dialkylaminoalkoxy, dialkylaminocarbonylalkoxy, dialkylaminocarbonyl, dialkylaminosulfonyl, —C(O)—OR^{a}, halogen, optionally substituted heterocyclyl, optionally substituted heterocyclylalkyl, optionally substituted heterocyclyloxy, hydroxy, hydroxyalkyl, nitro, oxo, optionally substituted phenyl, —SO₂CH₃, —SO₂CF₃, sulfonyl, tetrazolyl, thienylalkoxy, trifluoromethylcarbonylamino, trifluoromethylsulfonamido, heterocyclylalkoxy, heterocyclyl-S(O)ₚ, optionally substituted cycloalkyl-S(O)ₚ, optionally substituted alkyl-S—, optionally substituted heterocyclyl-S, heterocycloalkyl, cycloalkylalkyl, heterocycolthio, cycloalkylthio, N-alkylamino and N,N-dialkylamino where R^{a} is optionally substituted alkyl, optionally substituted heterocycloalkyl, or optionally substituted heterocyclyl and p is 1 or 2.

In a twentieth embodiment the invention provides a compound according to fourteenth through nineteenth embodiments wherein —Y—R³ is

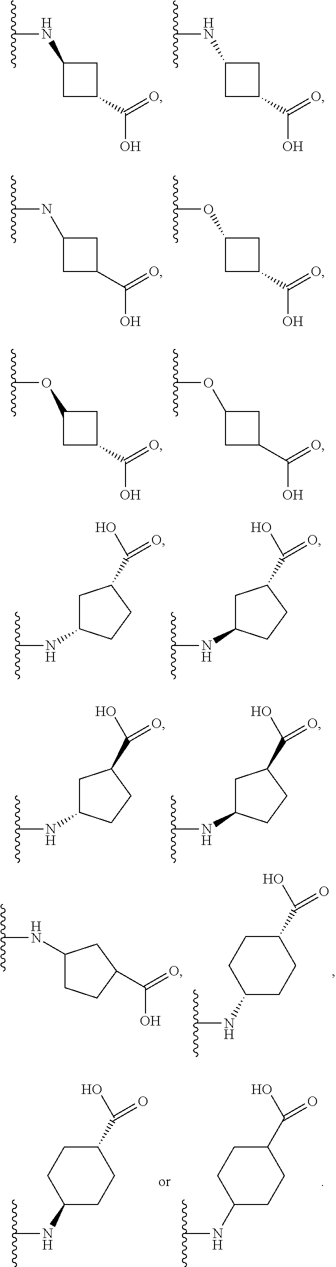

In a twenty-first embodiment the invention provides a compound according to fourteenth through twentieth embodiments wherein the compound is 3-{3-chloro-4-[5-(5-chloro-6-isopropoxy-pyridin-3-yl)-[1,2,4]oxadiazol-3-yl]-phenoxy}-cyclobutanecarboxylic acid;
(1R,3S)-3-(4-(5-(5-chloro-6-isopropoxypyridin-3-yl)-1,2,4-oxadiazol-3-yl)phenylamino)cyclopentanecarboxylic acid;
(1S,4R)-2-(3-(3-(5-chloro-6-isopropoxypyridin-3-yl)-1,2,4-oxadiazol-5-yl)-2-methylphenyl)-2-azabicyclo[2.2.1]heptan-3-one;
(1R,3S)-3-(4-(3-(5-chloro-6-isopropoxypyridin-3-yl)-1,2,4-oxadiazol-5-yl)-2-methylphenylamino)cyclopentanecarboxylic acid;
(1R,3S)-3-(4-(3-(5-chloro-6-isopropoxypyridin-3-yl)-1,2,4-oxadiazol-5-yl)-3-(trifluoromethyl)phenylamino)cyclopentanecarboxylic acid;
1-amino-3-(3-chloro-4-(5-(5-chloro-6-isopropoxypyridin-3-yl)-1,2,4-oxadiazol-3-yl)phenoxy)cyclopentanecarboxylic acid;
3-{3-chloro-4-[5-(5-chloro-6-isopropoxy-pyridin-3-yl)-[1,2,4]oxadiazol-3-yl]-phenoxy}-cyclobutanecarboxylic acid;
4-{3-chloro-4-[5-(5-chloro-6-isopropoxy-pyridin-3-yl)-[1,2,4]oxadiazol-3-yl]-phenoxy}-cyclohexanecarboxylic acid;
3-{3-chloro-4-[3-(5-chloro-6-isopropoxy-pyridin-3-yl)-[1,2,4]oxadiazol-5-yl]-phenoxy}-cyclobutanecarboxylic acid;
3-{3-chloro-4-[5-(5-chloro-6-isopropoxy-pyridin-3-yl)-[1,2,4]oxadiazol-3-yl]-phenoxy}-cyclobutanecarboxylic acid;
3-{3-chloro-4-[5-(5-chloro-6-isopropoxy-pyridin-3-yl)-[1,2,4]oxadiazol-3-yl]-phenoxy}-cyclohexanecarboxylic acid;
4-{3-chloro-4-[5-(5-chloro-6-isopropoxy-pyridin-3-yl)-[1,2,4]oxadiazol-3-yl]-phenoxy}-cyclohexanecarboxylic acid;
cis-{3-chloro-4-[5-(5-chloro-6-isopropoxy-pyridin-3-yl)-[1,2,4]oxadiazol-3-yl]-phenoxy}-cyclopentanecarboxylic acid;
trans-3-{3-chloro-4-[5-(5-chloro-6-isopropoxy-pyridin-3-yl)-[1,2,4]oxadiazol-3-yl]-phenoxy}-cyclopentanecarboxylic acid; or
(1R,3S)-3-(4-(5-(5-bromo-6-isopropoxypyridin-3-yl)-1,2,4-oxadiazol-3-yl)phenylamino)cyclopentanecarboxylic acid.

In a twenty-second embodiment the invention provides a compound according to Formula (V)

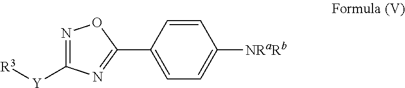

Y is a bond

R³ is -3-((1aS,5aR)-1,1,2-Trimethyl-1,1a,5,5a-tetrahydro-3-thia-cyclopropa[a]pentalenyl, or optionally substituted thienyl;

R^{a} is H or optionally substituted (C₁-C₆)alkyl

R^{b} is H, optionally substituted (C₁-C₆)alkyl or optionally substituted (C₃-C₆)cycloalkyl.

In a twenty-third embodiment the invention provides a compound according to the twenty-second embodiment wherein the compound is
(1R,3S)-3-(4-(3-(4-phenyl-5-(trifluoromethyl)thiophen-2-yl)-1,2,4-oxadiazol-5-yl)phenylamino)cyclopentanecarboxylic acid; or
(S)-3-{4-[3-((1aS,5aR)-1,1,2-trimethyl-1,1a,5,5a-tetrahydro-3-thia cyclopropa[a]pentalen-4-yl)-[1,2,4]oxadiazol-5-yl]-phenylamino}-cyclopentanecarboxylic acid.

In a twenty-fourth embodiment the invention provides a pharmaceutical composition comprising a compound according to any of the foregoing embodiments or a pharmaceutically acceptable salt, solvate, hydrate, metabolite, prodrug, enantiomer or stereoisomer thereof and a pharmaceutically acceptable diluent or carrier.

In a twenty-fifth embodiment the invention provides for the use of one or more compounds according to any of the foregoing embodiments or a pharmaceutically acceptable salt, solvate, hydrate, metabolite, prodrug or stereoisomer thereof for the manufacture of a medicament for treating an immune disorder.

In a twenty-sixth embodiment the invention provides for the use according the twenty-fifth embodiment wherein the immune disorder is active chronic hepatitis, Addison's Disease, ankylosing spondylitis, anti-phospholipid syndrome, asthma, atopic allergy, autoimmune atrophic gastritis, achlorhydra autoimmune, Celiac Disease, Crohn's Disease, Cushing's Syndrome, dermatomyositis, Goodpasture's Syndrome, Grave's Disease, Hashimoto's thyroiditis, idiopathic adrenal atrophy, idiopathic thrombocytopenia, juvenile rheumatoid arthritis, Lambert-Eaton Syndrome, lupoid hepatitis, lupus, mixed connective tissue disease, multiple sclerosis, pemphigoid, pemphigus vulgaris, pernicious anemia, phacogenic uveitis, polyarteritis nodosa, primary biliary cirrhosis, primary sclerosing cholangitis, psoriasis, Raynauds, Reiter's Syndrome, relapsing polychondritis, Schmidt's Syndrome, Sjogren's Syndrome, sympathetic ophthalmia, Takayasu's Arteritis, temporal arteritis, thyrotoxicosis, rheumatoid arthritis, Type B Insulin Resistance, ulcerative colitis, or Wegener's granulomatosis.

In a twenty-seventh embodiment the invention provides for the use of one or more compounds according to any of the foregoing embodiments or a pharmaceutically acceptable salt, solvate, hydrate, metabolite, prodrug, enantiomer or stereoisomer thereof for the manufacture of a medicament for treating a central nervous system disorder.

In a twenty-eighth embodiment the invention provides for the use of one or more compounds according to any of the foregoing embodiments or a pharmaceutically acceptable salt, solvate, hydrate, metabolite, prodrug, enantiomer or stereoisomer thereof for the manufacture of a medicament for treating multiple sclerosis.

In a twenty-ninth embodiment the invention provides for the use of one or more compounds according to claims any of the foregoing embodiments or a pharmaceutically acceptable salt, solvate, hydrate, metabolite, prodrug, enantiomer or stereoisomer thereof for the manufacture of a medicament for treating rheumatoid arthritis.

In a thirtieth embodiment the invention provides a pharmaceutical composition comprising one or more compounds according to Formula (I), (Ia), (II), (III), (IV), or (V) or pharmaceutically acceptable salts, solvates, hydrates, metabolites, prodrugs or stereoisomers thereof and a pharmaceutically acceptable diluent or carrier. In a preferred aspect, the invention provides a pharmaceutical composition wherein the compound or compounds are present in a therapeutically effective amount.

In a thirty-first embodiment the invention provides a packaged pharmaceutical comprising a one or more compounds according to Formula (I), (Ia), (II), (III), (IV), or (V) or pharmaceutically acceptable salts, solvates, hydrates, metabolites, prodrugs or stereoisomers thereof and instructions for use. In one embodiment, the invention provides a packaged pharmaceutical wherein the compound or compounds are present in a therapeutically effective amount. In another embodiment, the invention provides a packaged pharmaceutical wherein the compound or compounds are present in a prophylactically effective amount.

### DETAILED DESCRIPTION OF THE INVENTION

### Definitions

In this invention, the following definitions are applicable:

A “therapeutically effective amount” is an amount of a compound of Formula (I), (II), (III), (IV), or (V) or a combination of two or more such compounds, which inhibits, totally or partially, the progression of the condition or alleviates, at least partially, one or more symptoms of the condition. A therapeutically effective amount can also be an amount which is prophylactically effective. The amount which is therapeutically effective will depend upon the patient's size and gender, the condition to be treated, the severity of the condition and the result sought. For a given patient, a therapeutically effective amount can be determined by methods known to those of skill in the art.

“Physiologically acceptable salts” refers to those salts which retain the biological effectiveness and properties of the free bases and which are obtained by reaction with inorganic acids, for example, hydrochloric acid, hydrobromic acid, sulfuric acid, nitric acid, and phosphoric acid or organic acids such as sulfonic acid, carboxylic acid, organic phosphoric acid, methanesulfonic acid, ethanesulfonic acid, p-toluenesulfonic acid, citric acid, fumaric acid, maleic acid, succinic acid, benzoic acid, salicylic acid, lactic acid, tartaric acid (e.g. (+) or (−)-tartaric acid or mixtures thereof), amino acids (e.g. (+) or (−)-amino acids or mixtures thereof), and the like. These salts can be prepared by methods known to those skilled in the art.

Certain compounds of Formula (I), (II), (III), (IV), or (V) which have acidic substituents may exist as salts with pharmaceutically acceptable bases. The present invention includes such salts. Examples of such salts include sodium salts, potassium salts, lysine salts and arginine salts. These salts may be prepared by methods known to those skilled in the art.

Certain compounds of Formula (I), (II), (III), (IV), or (V), and their salts may exist in more than one crystal form and the present invention includes each crystal form and mixtures thereof.

Certain compounds of Formula (I), (II), (III), (IV), or (V) and their salts may also exist in the form of solvates, for example hydrates, and the present invention includes each solvate and mixtures thereof.

Certain compounds of Formula (I), (II), (III), (IV), or (V) may contain one or more chiral centers, and exist in different optically active forms. When compounds of Formula (I), (II), (III), (IV), or (V) contain one chiral center, the compounds exist in two enantiomeric forms and the present invention includes both enantiomers and mixtures of enantiomers, such as racemic mixtures. The enantiomers may be resolved by methods known to those skilled in the art, for example by formation of diastereoisomeric salts which may be separated, for example, by crystallization; formation of diastereoisomeric derivatives or complexes which may be separated, for example, by crystallization, gas-liquid or liquid chromatography; selective reaction of one enantiomer with an enantiomer-specific reagent, for example enzymatic esterification; or gas-liquid or liquid chromatography in a chiral environment, for example on a chiral support for example silica with a bound chiral ligand or in the presence of a chiral solvent. It will be appreciated that where the desired enantiomer is converted into another chemical entity by one of the separation procedures described above, a further step may be used to liberate the desired enantiomeric form. Alternatively, specific enantiomers may be synthesized by asymmetric synthesis using optically active reagents, substrates, catalysts or solvents, or by converting one enantiomer into the other by asymmetric transformation.

When a compound of Formula (I), (Ia), (II), (III), (IV), or (V) contains more than one chiral center, it may exist in diastereoisomeric forms. The diastereoisomeric compounds may be separated by methods known to those skilled in the art, for example chromatography or crystallization and the individual enantiomers may be separated as described above. The present invention includes each diastereoisomer of compounds of Formula (I), (Ia), (II), (III), (IV), or (V) and mixtures thereof.

Certain compounds of Formula (I), (Ia), (II), (III), (IV), and (V) may exist in different tautomeric forms or as different geometric isomers, and the present invention includes each tautomer and/or geometric isomer of compounds of Formula (I), (II), (III), (IV), and (V) and mixtures thereof.

Certain compounds of Formula (I), (Ia), (II), (III), (IV), and (V) may exist in different stable conformational forms which may be separable. Torsional asymmetry due to restricted rotation about an asymmetric single bond, for example because of steric hindrance or ring strain, may permit separation of different conformers. The present invention includes each conformational isomer of compounds of Formula (I), (Ia), (II), (III), (IV), and (V), and mixtures thereof.

Certain compounds of Formula (I), (Ia), (II), (III), (IV), and (V) may exist in zwitterionic form and the present invention includes each zwitterionic form of compounds of Formula (I), (Ia), (II), (III), (IV), and (V) and mixtures thereof.

As used herein the term “pro-drug” refers to an agent which is converted into the parent drug in vivo by some physiological chemical process (e.g., a prodrug on being brought to the physiological pH is converted to the desired drug form). Pro-drugs are often useful because, in some situations, they may be easier to administer than the parent drug. They may, for instance, be bioavailable by oral administration whereas the parent drug is not. The prodrug may also have improved solubility in pharmacological compositions over the parent drug. An example, without limitation, of a pro-drug would be a compound of the present invention wherein it is administered as an ester (the “pro-drug”) to facilitate transmittal across a cell membrane where water solubility is not beneficial, but then it is metabolically hydrolyzed to the carboxylic acid once inside the cell where water solubility is beneficial

Pro-drugs have many useful properties. For example, a pro-drug may be more water soluble than the ultimate drug, thereby facilitating intravenous administration of the drug. A pro-drug may also have a higher level of oral bioavailability than the ultimate drug. After administration, the prodrug is enzymatically or chemically cleaved to deliver the ultimate drug in the blood or tissue.

Exemplary pro-drugs upon cleavage release the corresponding free acid, and such hydrolyzable ester-forming residues of the compounds of this invention include but are not limited to carboxylic acid substituents (e.g., —(CH₂)C(O)OH or a moiety that contains a carboxylic acid) wherein the free hydrogen is replaced by (C₁-C₄)alkyl, (C₂-C₁₂)alkanoyloxymethyl, (C₄-C₉)1-(alkanoyloxy)ethyl, 1-methyl-1-(alkanoyloxy)-ethyl having from 5 to 10 carbon atoms, alkoxycarbonyloxymethyl having from 3 to 6 carbon atoms, 1-(alkoxycarbonyloxy)ethyl having from 4 to 7 carbon atoms, 1-methyl-1-(alkoxycarbonyloxy)ethyl having from 5 to 8 carbon atoms, N-(alkoxycarbonyl)aminomethyl having from 3 to 9 carbon atoms, 1-(N-(alkoxycarbonyl)amino)ethyl having from 4 to 10 carbon atoms, 3-phthalidyl, 4-crotonolactonyl, gamma-butyrolacton-4-yl, di-N,N—(C₁-C₂)alkylamino(C₂-C₃)alkyl (such as β-dimethylaminoethyl), carbamoyl-(C₁-C₂)alkyl, N,N-di(C₁-C₂)-alkylcarbamoyl-(C₁-C₂)alkyl and piperidino-, pyrrolidino- or morpholino(C₂-C₃)alkyl.

Other exemplary pro-drugs release an alcohol of Formula (I), (Ia), (II), (III), (IV), and (V) wherein the free hydrogen of the hydroxyl substituent (e.g., R¹ contains hydroxyl) is replaced by (C₁-C₆)alkanoyloxymethyl, 1-((C₁-C₆)alkanoyloxy)ethyl, 1-methyl-1-((C₁-C₆)alkanoyloxy)ethyl, (C₁-C₆)alkoxycarbonyloxymethyl, N—(C₁-C₆)alkoxycarbonylamino-methyl, succinoyl, (C₁-C₆)alkanoyl, α-amino(C₁-C₄)alkanoyl, arylactyl and α-aminoacyl, or α-aminoacyl-α-aminoacyl wherein said α-aminoacyl moieties are independently any of the naturally occurring L-amino acids found in proteins, P(O)(OH)₂, —P(O)(O(C₁-C₆)alkyl)₂ or glycosyl (the radical resulting from detachment of the hydroxyl of the hemiacetal of a carbohydrate).

The term “heterocyclic” or “heterocyclyl”, as used herein, include non-aromatic, ring systems, including, but not limited to, monocyclic, bicyclic and tricyclic rings, which can be completely saturated or which can contain one or more units of unsaturation, for the avoidance of doubt, the degree of unsaturation does not result in an aromatic ring system) and have 3 to 12 atoms including at least one heteroatom, such as nitrogen, oxygen, or sulfur. For purposes of exemplification, which should not be construed as limiting the scope of this invention, the following are examples of heterocyclic rings: azabicyclo[2.2.1]heptanyl, azepinyl, azetidinyl, morpholinyl, oxopiperidinyl, oxopyrrolidinyl, piperazinyl, piperidinyl, pyrrolidinyl, quinicludinyl, thiomorpholinyl, tetrahydropyranyl and tetrahydrofuranyl.

The term “heteroaryl” as used herein, include aromatic ring systems, including, but not limited to, monocyclic, bicyclic and tricyclic rings, and have 3 to 12 atoms including at least one heteroatom, such as nitrogen, oxygen, or sulfur. For purposes of exemplification, which should not be construed as limiting the scope of this invention: azaindolyl, benzo[b]thienyl, benzimidazolyl, benzofuranyl, benzoxazolyl, benzothiazolyl, benzothiadiazolyl, benzotriazolyl, benzoxadiazolyl, furanyl, imidazolyl, imidazopyridinyl, indolyl, indolinyl, indazolyl, isoindolinyl, isoxazolyl, isothiazolyl, isoquinolinyl, oxadiazolyl, oxazolyl, purinyl, pyranyl, pyrazinyl, pyrazolyl, pyridinyl, pyrimidinyl, pyrrolyl, pyrrolo[2,3-d]pyrimidinyl, pyrazolo[3,4-d]pyrimidinyl, quinolinyl, quinazolinyl, triazolyl, thiazolyl, thiophenyl, tetrahydroindolyl, tetrazolyl, thiadiazolyl, thienyl, thiomorpholinyl, triazolyl or tropanyl.

When the term “substituted heterocyclic” (or heterocyclyl) or “substituted heteroaryl” or “substituted aryl” is used, what is meant is that the heterocyclic, heteroaryl or aryl group is substituted with one or more substituents that can be made by one of ordinary skill in the art and results in a molecule that is an agonist or antagonist of the sphingosine receptor family. For purposes of exemplification, which should not be construed as limiting the scope of this invention, preferred substituents for the heterocycle, heteroaryl or aryl group of this invention are each independently selected from the optionally substituted group consisting of alkenyl, alkoxy, alkoxyalkoxy, alkoxyalkyl, alkoxycarbonyl, alkoxycarbonylheterocycloalkoxy, alkyl, alkylamino, alkylcarbonyl, alkylester, alkyl-NH-alkyl, -alkyl-NH-cycloalkyl, alkyl-O—C(O)—, -alkyl-heterocyclyl, -alkyl-cycloalkyl, alkyl-nitrile, alkynyl, amido groups, amino, aminoalkyl, aminocarbonyl, carbonitrile, carbonylalkoxy, carboxamido, CF₃, CN, —C(O)OH, —C(O)H, —C(O)—C(CH₃)₃, —OH, —C(O)O-alkyl, —C(O)O-cycloalkyl, —C(O)O-heterocyclyl, —C(O)O-alkyl-aryl, —C(O)-alkyl, —C(O)-cycloalkyl, —C(O)-heterocyclyl, cycloalkyl, dialkylaminoalkoxy, dialkylaminocarbonylalkoxy, dialkylaminocarbonyl, halogen, heterocyclyl, a heterocycloalkyl group, heterocyclyloxy, hydroxy, hydroxyalkyl, nitro, OCF₃, oxo, —O-alkyl, —O-heteroaryl, —O-heterocyclyl, —SO₂CH₃, —SO₂NH₂, —SO₂NH-alkyl, —SO₂N(alkyl)₂, tetrazolyl, thienylalkoxy, trifluoromethylcarbonylamino, trifluoromethylsulfonamido, heterocyclylalkoxy, heterocyclyl-S(O)ₚ, cycloalkyl-S(O)ₚ, alkyl-S—, heterocyclyl-S, heterocycloalkyl, cycloalkylalkyl, heterocycolthio, cycloalkylthio, —Z¹⁰⁵—C(O)N(R)₂, —Z¹⁰⁵—N(R)—C(O)—Z²⁰⁰, —Z¹⁰⁵—N(R)—S(O)₂Z²⁰⁰, —Z¹⁰⁵—N(R)—C(O)—N(R)—Z²⁰⁰, —N(R), —N(H)-alkyl, —N(H)-cycloalkyl, —C(O)R, —N(R)—C(O)OR, OR—C(O)-heterocyclyl-OR, R_{c} and —CH₂OR_{c};
wherein p is 0, 1 or 2;
where R_{c} for each occurrence is independently hydrogen, optionally substituted alkyl, optionally substituted aryl, —(C₁-C₆)—NR_{d}Rₑ, -E-(CH₂)ₜ—NR_{d}Rₑ, -E-(CH₂)ₜ—O-alkyl, -E-(CH₂)ₜ—S-alkyl, or -E-(CH₂)ₜ—OH;
   wherein t is an integer from about 1 to about 6;
Z¹⁰⁵ for each occurrence is independently a covalent bond, alkyl, alkenyl or alkynyl; and
Z²⁰⁰ for each occurrence is independently selected from an optionally substituted group selected from the group consisting of alkyl, alkenyl, alkynyl, phenyl, alkyl-phenyl, alkenyl-phenyl or alkynyl-phenyl;
E is a direct bond, O, S, S(O), S(O)₂, or NR_{f}, wherein R_{f} is H or alkyl and R_{d} and Rₑ are independently H, alkyl, alkanoyl or SO₂-alkyl; or R_{d}, Rₑ and the nitrogen atom to which they are attached together to form a five- or six-membered heterocyclic ring.

An “heterocycloalkyl” group, as used herein, is a heterocyclic group that is linked to a compound by an aliphatic group having from one to about eight carbon atoms. For example, a morpholinomethyl group is an heterocycloalkyl group.

As used herein, “aliphatic” or “an aliphatic group” or notations such as “(C₀-C₈)” include straight chained or branched hydrocarbons which are completely saturated or which contain one or more units of unsaturation, and, thus, includes alkyl, alkenyl, alkynyl and hydrocarbons comprising a mixture of single, double and triple bonds. When the group is a C₀ it means that the moiety is not present or in other words, it is a bond. As used herein, “alkyl” means C₁-C₈ and includes straight chained or branched hydrocarbons, which are completely saturated. Examples of alkyls are methyl, ethyl, propyl, butyl, pentyl, hexyl and isomers thereof. As used herein, “alkenyl” and “alkynyl” means C₂-C₈ and includes straight chained or branched hydrocarbons which contain one or more units of unsaturation, one or more double bonds for alkenyl and one or more triple bonds for alkynyl.

As used herein, aromatic groups (or aryl groups) include aromatic carbocyclic ring systems (e.g. phenyl and cyclopentyldienyl) and fused polycyclic aromatic ring systems (e.g. naphthyl, biphenylenyl and 1,2,3,4-tetrahydronaphthyl).

As used herein, cycloalkyl means C₃-C₁₂ monocyclic or multicyclic (e.g., bicyclic, tricyclic, etc.) hydrocarbons that is completely saturated or has one or more unsaturated bonds but does not amount to an aromatic group. Examples of a cycloalkyl group are cyclopropyl, cyclobutyl, cyclopentyl, cyclopentenyl, cyclohexyl and cyclohexenyl.

As used herein, many moieties or substituents are termed as being either “substituted” or “optionally substituted”. When a moiety is modified by one of these terms, unless otherwise noted, it denotes that any portion of the moiety that is known to one skilled in the art as being available for substitution can be substituted, which includes one or more substituents, where if more than one substituent then each substituent is independently selected. Such means for substitution are well-known in the art and/or taught by the instant disclosure. For purposes of exemplification, which should not be construed as limiting the scope of this invention, some examples of groups that are substituents are: alkenyl groups, alkoxy group (which itself can be substituted, such as —O—C₁-C₆-alkyl-OR, —O—C₁-C₆-alkyl-N(R)₂, and OCF₃), alkoxyalkoxy, alkoxycarbonyl, alkoxycarbonylpiperidinyl-alkoxy, alkyl groups (which itself can also be substituted, such as —C₁-C₆-alkyl-OR, —C₁-C₆-alkyl-N(R)₂, COOH, and —CF₃), alkylamino, alkylcarbonyl, alkylester, alkylnitrile, alkylsulfonyl, amino, aminoalkoxy, CF₃, COH, COOH, CN, cycloalkyl, dialkylamino, dialkylaminoalkoxy, dialkylamino carbonyl, dialkylaminocarbonylalkoxy, dialkylaminosulfonyl, esters (—C(O)—OR, where R is groups such as alkyl, heterocycloalkyl (which can be substituted), heterocyclyl, etc., which can be substituted), halogen or halo group (F, Cl, Br, I), hydroxy, morpholinoalkoxy, morpholinoalkyl, —NH—C₁-C₆-alkyl-COOH, nitro, oxo, OCF₃, S(O)₂CH₃, S(O)₂CF₃, and sulfonyl, N-alkylamino or N,N-dialkylamino (in which the alkyl groups can also be substituted).

### Methods of Use

The present invention provides compounds described by general Formula (I), (Ia), (II), (III), (IV), and (V), which are effective as antagonists or agonists of the G protein-coupled S1P receptor family. These compounds reduce the number of circulating and infiltrating T- and B-lymphocytes affording a beneficial immunosuppressive effect.

The present invention also provides compounds that exhibit activity within the S1P receptor family.

In a related aspect the invention provides a method for modulating receptors of the S1P family in a human subject suffering from a disorder in which modulation of S1P activity is beneficial, comprising administering to the human subject a compound of Formula (I), (Ia), (II), (III), (IV), and (V) such that modulation of S1P activity in the human subject is triggered and treatment is achieved.

In another related aspect the invention provides a method of modulating sphingosine 1-phosphate receptor 1 activity comprising contacting a cell with one or more compounds of Formula (I), (Ia), (II), (III), (IV), and (V).

A compound of Formula (I), (Ia), (II), (III), (IV), and (V) or a salt thereof or pharmaceutical compositions containing a therapeutically effective amount thereof is useful in the treatment of a disorder selected from the group comprising CNS system disorders, arthritis, rheumatoid arthritis, osteoarthritis, ankylosing spondylitis, juvenile chronic arthritis, Lyme arthritis, psoriatic arthritis, reactive arthritis, and septic arthritis, spondyloarthropathy, systemic lupus erythematosus, Crohn's disease, ulcerative colitis, inflammatory bowel disease, insulin dependent diabetes mellitus, thyroiditis, asthma, allergic diseases, psoriasis, dermatitis scleroderma, graft versus host disease, organ transplant rejection (including but not limited to bone marrow and solid organ rejection), acute or chronic immune disease associated with organ transplantation, sarcoidosis, atherosclerosis, disseminated intravascular coagulation, Kawasaki's disease, Grave's disease, nephrotic syndrome, chronic fatigue syndrome, Wegener's granulomatosis, Henoch-Schoenlein purpurea, microscopic vasculitis of the kidneys, chronic active hepatitis, uveitis, septic shock, toxic shock syndrome, sepsis syndrome, cachexia, infectious diseases, parasitic diseases, acquired immunodeficiency syndrome, acute transverse myelitis, Huntington's chorea, Parkinson's disease, Alzheimer's disease, stroke, primary biliary cirrhosis, hemolytic anemia, malignancies, heart failure, myocardial infarction, Addison's disease, sporadic, polyglandular deficiency type I and polyglandular deficiency type II, Schmidt's syndrome, adult (acute) respiratory distress syndrome, alopecia, alopecia greata, seronegative arthopathy, arthropathy, Reiter's disease, psoriatic arthropathy, ulcerative colitic arthropathy, enteropathic synovitis, chlamydia, yersinia and salmonella associated arthropathy, atheromatous disease/arteriosclerosis, atopic allergy, autoimmune bullous disease, pemphigus vulgaris, pemphigus foliaceus, pemphigoid, linear IgA disease, autoimmune haemolytic anaemia, Coombs positive haemolytic anaemia, acquired pernicious anaemia, juvenile pernicious anaemia, myalgic encephalitis/Royal Free Disease, chronic mucocutaneous candidiasis, giant cell arteritis, primary sclerosing hepatitis, cryptogenic autoimmune hepatitis, Acquired Immunodeficiency Disease Syndrome, Acquired Immunodeficiency Related Diseases, Hepatitis B, Hepatitis C, common varied immunodeficiency (common variable hypogammaglobulinaemia), dilated cardiomyopathy, female infertility, ovarian failure, premature ovarian failure, fibrotic lung disease, chronic wound healing, cryptogenic fibrosing alveolitis, post-inflammatory interstitial lung disease, interstitial pneumonitis, connective tissue disease associated interstitial lung disease, mixed connective tissue disease associated lung disease, systemic sclerosis associated interstitial lung disease, rheumatoid arthritis associated interstitial lung disease, systemic lupus erythematosus associated lung disease, dermatomyositis/polymyositis associated lung disease, Sjögren's disease associated lung disease, ankylosing spondylitis associated lung disease, vasculitic diffuse lung disease, haemosiderosis associated lung disease, drug-induced interstitial lung disease, radiation fibrosis, bronchiolitis obliterans, chronic eosinophilic pneumonia, lymphocytic infiltrative lung disease, postinfectious interstitial lung disease, gouty arthritis, autoimmune hepatitis, type-1 autoimmune hepatitis (classical autoimmune or lupoid hepatitis), type-2 autoimmune hepatitis (anti-LKM antibody hepatitis), autoimmune mediated hypoglycaemia, type B insulin resistance with acanthosis nigricans, hypoparathyroidism, acute immune disease associated with organ transplantation, chronic immune disease associated with organ transplantation, osteoarthrosis, primary sclerosing cholangitis, psoriasis type 1, psoriasis type 2, idiopathic leucopaenia, autoimmune neutropaenia, renal disease NOS, glomerulonephritides, microscopic vasculitis of the kidneys, Lyme disease, discoid lupus erythematosus, male infertility idiopathic or NOS, sperm autoimmunity, multiple sclerosis (all subtypes), sympathetic ophthalmia, pulmonary hypertension secondary to connective tissue disease, Goodpasture's syndrome, pulmonary manifestation of polyarteritis nodosa, acute rheumatic fever, rheumatoid spondylitis, Still's disease, systemic sclerosis, Sjögren's syndrome, Takayasu's disease/arteritis, autoimmune thrombocytopaenia, idiopathic thrombocytopaenia, autoimmune thyroid disease, hyperthyroidism, goitrous autoimmune hypothyroidism (Hashimoto's disease), atrophic autoimmune hypothyroidism, primary myxoedema, phacogenic uveitis, primary vasculitis, vitiligo, acute liver disease, chronic liver diseases, alcoholic cirrhosis, alcohol-induced liver injury, choleosatatis, idiosyncratic liver disease, Drug-Induced hepatitis, Non-alcoholic Steatohepatitis, allergy and asthma, group B streptococci (GBS) infection, mental disorders (e.g., depression and schizophrenia), Th2 Type and Th1 Type mediated diseases, acute and chronic pain (different forms of pain), and cancers such as lung, breast, stomach, bladder, colon, pancreas, ovarian, prostate and rectal cancer and hematopoietic malignancies (leukemia and lymphoma), and hematopoietic malignancies (leukemia and lymphoma), Abetalipoprotemia, Acrocyanosis, acute and chronic parasitic or infectious processes, acute leukemia, acute lymphoblastic leukemia (ALL), acute myeloid leukemia (AML), acute or chronic bacterial infection, acute pancreatitis, acute renal failure, adenocarcinomas, aerial ectopic beats, AIDS dementia complex, alcohol-induced hepatitis, allergic conjunctivitis, allergic contact dermatitis, allergic rhinitis, allograft rejection, alpha-1-antitrypsin deficiency, amyotrophic lateral sclerosis, anemia, angina pectoris, anterior horn cell degeneration, anti cd3 therapy, antiphospholipid syndrome, anti-receptor hypersensitivity reactions, aordic and peripheral aneurysms, aortic dissection, arterial hypertension, arteriosclerosis, arteriovenous fistula, ataxia, atrial fibrillation (sustained or paroxysmal), atrial flutter, atrioventricular block, B cell lymphoma, bone graft rejection, bone marrow transplant (BMT) rejection, bundle branch block, Burkitt's lymphoma, Burns, cardiac arrhythmias, cardiac stun syndrome, cardiac tumors, cardiomyopathy, cardiopulmonary bypass inflammation response, cartilage transplant rejection, cerebellar cortical degenerations, cerebellar disorders, chaotic or multifocal atrial tachycardia, chemotherapy associated disorders, chromic myelocytic leukemia (CML), chronic alcoholism, chronic inflammatory pathologies, chronic lymphocytic leukemia (CLL), chronic obstructive pulmonary disease (COPD), chronic salicylate intoxication, colorectal carcinoma, congestive heart failure, conjunctivitis, contact dermatitis, cor pulmonale, coronary artery disease, Creutzfeldt-Jakob disease, culture negative sepsis, cystic fibrosis, cytokine therapy associated disorders, Dementia pugilistica, demyelinating diseases, dengue hemorrhagic fever, dermatitis, dermatologic conditions, diabetes, diabetes mellitus, diabetic ateriosclerotic disease, Diffuse Lewy body disease, dilated congestive cardiomyopathy, disorders of the basal ganglia, Down's Syndrome in middle age, drug-induced movement disorders induced by drugs which block CNS dopamine receptors, drug sensitivity, eczema, encephalomyelitis, endocarditis, endocrinopathy, epiglottitis, epstein-barr virus infection, erythromelalgia, extrapyramidal and cerebellar disorders, familial hematophagocytic lymphohistiocytosis, fetal thymus implant rejection, Friedreich's ataxia, functional peripheral arterial disorders, fungal sepsis, gas gangrene, gastric ulcer, glomerular nephritis, graft rejection of any organ or tissue, gram negative sepsis, gram positive sepsis, granulomas due to intracellular organisms, hairy cell leukemia, Hallervorden-Spatz disease, hashimoto's thyroiditis, hay fever, heart transplant rejection, hemachromatosis, hemodialysis, hemolytic uremic syndrome/thrombolytic thrombocytopenic purpura, hemorrhage, hepatitis (A), His bundle arrhythmias, HIV infection/HIV neuropathy, Hodgkin's disease, hyperkinetic movement disorders, hypersensitivity reactions, hypersensitivity pneumonitis, hypertension, hypokinetic movement disorders, hypothalamic-pituitary-adrenal axis evaluation, idiopathic Addison's disease, idiopathic pulmonary fibrosis, antibody mediated cytotoxicity, Asthenia, infantile spinal muscular atrophy, inflammation of the aorta, influenza a, ionizing radiation exposure, iridocyclitis/uveitis/optic neuritis, ischemia-reperfusion injury, ischemic stroke, juvenile rheumatoid arthritis, juvenile spinal muscular atrophy, Kaposi's sarcoma, kidney transplant rejection, legionella, leishmaniasis, leprosy, lesions of the corticospinal system, lipedema, liver transplant rejection, lymphederma, malaria, malignant Lymphoma, malignant histiocytosis, malignant melanoma, meningitis, meningococcemia, metabolic/idiopathic, migraine headache, mitochondrial multi.system disorder, mixed connective tissue disease, monoclonal gammopathy, multiple myeloma, multiple systems degenerations (Mencel Dejerine-Thomas Shi-Drager and Machado-Joseph), myasthenia gravis, mycobacterium avium intracellulare, mycobacterium tuberculosis, myelodyplastic syndrome, myocardial infarction, myocardial ischemic disorders, nasopharyngeal carcinoma, neonatal chronic lung disease, nephritis, nephrosis, neurodegenerative diseases, neurogenic I muscular atrophies, neutropenic fever, non-hodgkins lymphoma, occlusion of the abdominal aorta and its branches, occlusive arterial disorders, okt3 therapy, orchitis/epidydimitis, orchitis/vasectomy reversal procedures, organomegaly, osteoporosis, pancreas transplant rejection, pancreatic carcinoma, paraneoplastic syndrome/hypercalcemia of malignancy, parathyroid transplant rejection, pelvic inflammatory disease, perennial rhinitis, pericardial disease, peripheral atherlosclerotic disease, peripheral vascular disorders, peritonitis, pernicious anemia, pneumocystis carinii pneumonia, pneumonia, POEMS syndrome (polyneuropathy, organomegaly, endocrinopathy, monoclonal gammopathy, and skin changes syndrome), post perfusion syndrome, post pump syndrome, post-MI cardiotomy syndrome, preeclampsia, Progressive supranucleo Palsy, primary pulmonary hypertension, radiation therapy, Raynaud's phenomenon and disease, Raynaud's disease, Refsum's disease, regular narrow QRS tachycardia, renovascular hypertension, reperfusion injury, restrictive cardiomyopathy, sarcomas, scleroderma, senile chorea, Senile Dementia of Lewy body type, seronegative arthropathies, shock, sickle cell anemia, skin allograft rejection, skin changes syndrome, small bowel transplant rejection, solid tumors, specific arrythmias, spinal ataxia, spinocerebellar degenerations, streptococcal myositis, structural lesions of the cerebellum, Subacute sclerosing panencephalitis, Syncope, syphilis of the cardiovascular system, systemic anaphalaxis, systemic inflammatory response syndrome, systemic onset juvenile rheumatoid arthritis, T-cell or FAB ALL, Telangiectasia, thromboangitis obliterans, thrombocytopenia, toxicity, transplants, trauma/hemorrhage, type III hypersensitivity reactions, type IV hypersensitivity, unstable angina, uremia, urosepsis, urticaria, valvular heart diseases, varicose veins, vasculitis, venous diseases, venous thrombosis, ventricular fibrillation, viral and fungal infections, vital encephalitis/aseptic meningitis, vital-associated hemaphagocytic syndrome, Wernicke-Korsakoff syndrome, Wilson's disease, xenograft rejection of any organ or tissue, and diseases involving inappropriate vascularization for example diabetic retinopathy, retinopathy of prematurity, choroidal neovascularization due to age-related macular degeneration, and infantile hemangiomas in human beings. In addition, such compounds may be useful in the treatment of disorders such as, edema, ascites, effusions, and exudates, including for example macular edema, cerebral edema, acute lung injury, adult respiratory distress syndrome (ARDS), proliferative disorders such as restenosis, fibrotic disorders such as hepatic cirrhosis and atherosclerosis, mesangial cell proliferative disorders such as glomerulonephritis, diabetic nephropathy, malignant nephrosclerosis, thrombotic microangiopathy syndromes, and glomerulopathies, myocardial angiogenesis, coronary and cerebral collaterals, ischemic limb angiogenesis, ischemia/reperfusion injury, peptic ulcer Helicobacter related diseases, virally-induced angiogenic disorders, Crow-Fukase syndrome (POEMS), preeclampsia, menometrorrhagia, cat scratch fever, rubeosis, neovascular glaucoma and retinopathies such as those associated with diabetic retinopathy, retinopathy of prematurity, age-related macular degeneration or a central nervous system disorder. In addition, these compounds can be used as active agents against solid tumors, malignant ascites, von Hippel Lindau disease, hematopoietic cancers and hyperproliferative disorders such as thyroid hyperplasia (especially Grave's disease), and cysts (such as hypervascularity of ovarian stroma characteristic of polycystic ovarian syndrome (Stein-Leventhal syndrome) and polycystic kidney disease since such diseases require a proliferation of blood vessel cells for growth and/or metastasis.

### Combination Therapy

Compounds of Formula (I), (Ia), (II), (III), (IV), and (V) of the invention can be used alone or in combination with another therapeutic agent to treat such diseases. It should be understood that the compounds of the invention can be used alone or in combination with an additional agent, e.g., a therapeutic agent, said additional agent being selected by the skilled artisan for its intended purpose. For example, the additional agent can be a therapeutic agent art-recognized as being useful to treat the disease or condition being treated by the compound of the present invention. The additional agent also can be an agent that imparts a beneficial attribute to the therapeutic composition e.g., an agent that affects the viscosity of the composition.

It should further be understood that the combinations which are to be included within this invention are those combinations useful for their intended purpose. The agents set forth below are illustrative for purposes and not intended to be limited. The combinations, which are part of this invention, can be the compounds of the present invention and at least one additional agent selected from the lists below. The combination can also include more than one additional agent, e.g., two or three additional agents if the combination is such that the formed composition can perform its intended function.

Preferred combinations are non-steroidal anti-inflammatory drug(s) also referred to as NSAIDS which include drugs like ibuprofen. Other preferred combinations are corticosteroids including prednisolone; the well known side-effects of steroid use can be reduced or even eliminated by tapering the steroid dose required when treating patients in combination with the S1P receptor agonists or antagonists of this invention. Non-limiting examples of therapeutic agents for rheumatoid arthritis with which a compound of Formula (I), (Ia), (II), (III), (IV), and (V), of the invention can be combined include the following: cytokine suppressive anti-inflammatory drug(s) (CSAIDs); antibodies to or antagonists of other human cytokines or growth factors, for example, TNF, LT, IL-1, IL-2, IL-3, IL-4, IL-5, IL-6, IL-7, IL-8, IL-12, IL-15, IL-16, IL-21, IL-23, interferons, EMAP-II, GM-CSF, FGF, and PDGF. S/T kinase inhibitors of the invention can be combined with antibodies to cell surface molecules such as CD2, CD3, CD4, CD8, CD25, CD28, CD30, CD40, CD45, CD69, CD80 (B7.1), CD86 (B7.2), CD90, CTLA or their ligands including CD154 (gp39 or CD40L).

Preferred combinations of therapeutic agents may interfere at different points in the autoimmune and subsequent inflammatory cascade; preferred examples include TNF antagonists like chimeric, humanized or human TNF antibodies, D2E7 (HUMIRA™), (PCT Publication No. WO 97/29131), CA2 (REMICADE™), CDP 571, and soluble p55 or p75 TNF receptors, derivatives, thereof, (p75TNFR1gG (ENBREL™) or p55TNFR1gG (Lenercept), and also TNFα converting enzyme (TACE) inhibitors; similarly IL-1 inhibitors (Interleukin-1-converting enzyme inhibitors, IL-1RA etc.) may be effective for the same reason. Other preferred combinations include Interleukin 11. Yet other preferred combinations are the other key players of the autoimmune response which may act parallel to, dependent on or in concert with IL-18 function; especially preferred are IL-12 antagonists including IL-12 antibodies or soluble IL-12 receptors, or IL-12 binding proteins. It has been shown that IL-12 and IL-18 have overlapping but distinct functions and a combination of antagonists to both may be most effective. Yet another preferred combination are non-depleting anti-CD4 inhibitors. Yet other preferred combinations include antagonists of the co-stimulatory pathway CD80 (B7.1) or CD86 (B7.2) including antibodies, soluble receptors or antagonistic ligands.

A compound of Formula (I), (Ia), (II), (III), (IV), and (V) of the invention may also be combined with agents, such as methotrexate, 6-MP, azathioprine sulphasalazine, mesalazine, olsalazine chloroquinine/hydroxychloroquine, pencillamine, aurothiomalate (intramuscular and oral), azathioprine, cochicine, corticosteroids (oral, inhaled and local injection), beta-2 adrenoreceptor agonists (salbutamol, terbutaline, salmeteral), xanthines (theophylline, aminophylline), cromoglycate, nedocromil, ketotifen, ipratropium and oxitropium, cyclosporin, FK506, rapamycin, mycophenolate mofetil, leflunomide, NSAIDs, for example, ibuprofen, corticosteroids such as prednisolone, phosphodiesterase inhibitors, adensosine agonists, antithrombotic agents, complement inhibitors, adrenergic agents, agents which interfere with signalling by proinflammatory cytokines such as TNF or IL-1 (e.g. IRAK, NIK, IKK, p38 or MAP kinase inhibitors), IL-1β converting enzyme inhibitors, T-cell signalling inhibitors such as kinase inhibitors, metalloproteinase inhibitors, sulfasalazine, 6-mercaptopurines, angiotensin converting enzyme inhibitors, soluble cytokine receptors and derivatives thereof (e.g. soluble p55 or p75 TNF receptors and the derivatives p75TNFRIgG (Enbrel™ and p55TNFRIgG (Lenercept)), sIL-1RI, sIL-1RII, sIL-6R), antiinflammatory cytokines (e.g. IL-4, IL-10, IL-11, IL-13 and TGFβ), celecoxib, folic acid, hydroxychloroquine sulfate, rofecoxib, etanercept, infliximab, naproxen, valdecoxib, sulfasalazine, methylprednisolone, meloxicam, methylprednisolone acetate, gold sodium thiomalate, aspirin, triamcinolone acetonide, propoxyphene napsylate/apap, folate, nabumetone, diclofenac, piroxicam, etodolac, diclofenac sodium, oxaprozin, oxycodone HCl, hydrocodone bitartrate/apap, diclofenac sodium/misoprostol, fentanyl, anakinra, tramadol HCl, salsalate, sulindac, cyanocobalamin/fa/pyridoxine, acetaminophen, alendronate sodium, prednisolone, morphine sulfate, lidocaine hydrochloride, indomethacin, glucosamine sulf/chondroitin, amitriptyline HCl, sulfadiazine, oxycodone HCl/acetaminophen, olopatadine HCl misoprostol, naproxen sodium, omeprazole, cyclophosphamide, rituximab, IL-1 TRAP, MRA, CTLA4-IG, IL-18 BP, anti-IL-12, Anti-IL15, BIRB-796, SCIO-469, VX-702, AMG-548, VX-740, Roflumilast, IC-485, CDC-801, and Mesopram. Preferred combinations include methotrexate or leflunomide and in moderate or severe rheumatoid arthritis cases, cyclosporine and anti-TNF antibodies as noted above.

Non-limiting examples of therapeutic agents for inflammatory bowel disease with which a compound of Formula (I), (Ia), (II), (III), (IV), or (V) of the invention can be combined include the following: budenoside; epidermal growth factor; corticosteroids; cyclosporin, sulfasalazine; aminosalicylates; 6-mercaptopurine; azathioprine; metronidazole; lipoxygenase inhibitors; mesalamine; olsalazine; balsalazide; antioxidants; thromboxane inhibitors; IL-1 receptor antagonists; anti-IL-1β monoclonal antibodies; anti-IL-6 monoclonal antibodies; growth factors; elastase inhibitors; pyridinyl-imidazole compounds; antibodies to or antagonists of other human cytokines or growth factors, for example, TNF, LT, IL-1, IL-2, IL-6, IL-7, IL-8, IL-12, IL-15, IL-16, EMAP-II, GM-CSF, FGF, and PDGF; cell surface molecules such as CD2, CD3, CD4, CD8, CD25, CD28, CD30, CD40, CD45, CD69, CD90 or their ligands; methotrexate; cyclosporine; FK506; rapamycin; mycophenolate mofetil; leflunomide; NSAIDs, for example, ibuprofen; corticosteroids such as prednisolone; phosphodiesterase inhibitors; adenosine agonists; antithrombotic agents; complement inhibitors; adrenergic agents; agents which interfere with signalling by proinflammatory cytokines such as TNF [symbol] or IL-1 (e.g. IRAK, NIK, IKK, or MAP kinase inhibitors); IL-1β converting enzyme inhibitors; TNF [symbol] converting enzyme inhibitors; T-cell signalling inhibitors such as kinase inhibitors; metalloproteinase inhibitors; sulfasalazine; azathioprine; 6-mercaptopurines; angiotensin converting enzyme inhibitors; soluble cytokine receptors and derivatives thereof (e.g. soluble p55 or p75 TNF receptors, sIL-1RI, sIL-1RII, sIL-6R) and antiinflammatory cytokines (e.g. IL-4, IL-10, IL-11, IL-13 and TGFβ). Preferred examples of therapeutic agents for Crohn's disease with which a compound of Formula (I), (Ia), (II), (III), (IV), or (V) can be combined include the following: TNF antagonists, for example, anti-TNF antibodies, D2E7 (PCT Publication No. WO 97/29131; HUMIRA™), CA2 (REMICADE™), CDP 571, TNFR-Ig constructs, (p75TNFRIgG (ENBREL™) and p55TNFRIgG (Lenercept™)) inhibitors and PDE4 inhibitors. A compound of Formula (I), (Ia), (II), (III), (IV), or (V) can be combined with corticosteroids, for example, budenoside and dexamethasone; sulfasalazine, 5-aminosalicylic acid; olsalazine; and agents which interfere with synthesis or action of proinflammatory cytokines such as IL-1, for example, IL-1β converting enzyme inhibitors and IL-1ra; T cell signaling inhibitors, for example, tyrosine kinase inhibitors 6-mercaptopurines; IL-11; mesalamine; prednisone; azathioprine; mercaptopurine; infliximab; methylprednisolone sodium succinate; diphenoxylate/atrop sulfate; loperamide hydrochloride; methotrexate; omeprazole; folate; ciprofloxacin/dextrose-water; hydrocodone bitartrate/apap; tetracycline hydrochloride; fluocinonide; metronidazole; thimerosal/boric acid; cholestyramine/sucrose; ciprofloxacin hydrochloride; hyoscyamine sulfate; meperidine hydrochloride; midazolam hydrochloride; oxycodone HCl/acetaminophen; promethazine hydrochloride; sodium phosphate; sulfamethoxazole/trimethoprim; celecoxib; polycarbophil; propoxyphene napsylate; hydrocortisone; multivitamins; balsalazide disodium; codeine phosphate/apap; colesevelam HCl; cyanocobalamin; folic acid; levofloxacin; methylprednisolone; natalizumab and interferon-gamma.

Non-limiting examples of therapeutic agents for multiple sclerosis with which a compound of Formula (I), (Ia), (II), (III), (IV), or (V) can be combined include the following: corticosteroids; prednisolone; methylprednisolone; azathioprine; cyclophosphamide; cyclosporine; methotrexate; 4-aminopyridine; tizanidine; interferon-β1a (Avonex®; Biogen); interferon-β1b (Betaseron®; Chiron/Berlex); interferon α-n3) (Interferon Sciences/Fujimoto), interferon-α (Alfa Wassermann/J&J), interferon β1A-IF (Serono/Inhale Therapeutics), Peginterferon α 2b (Enzon/Schering-Plough), Copolymer 1 (Cop-1; Copaxone®; Teva Pharmaceutical Industries, Inc.); hyperbaric oxygen; intravenous immunoglobulin; clabribine; antibodies to or antagonists of other human cytokines or growth factors and their receptors, for example, TNF, LT, IL-1, IL-2, IL-6, IL-7, IL-8, IL-12, IL-23, IL-15, IL-16, EMAP-II, GM-CSF, FGF, and PDGF. A compound of Formula (I), (Ia), (II), (III), (IV), or (V) can be combined with antibodies to cell surface molecules such as CD2, CD3, CD4, CD8, CD19, CD20, CD25, CD28, CD30, CD40, CD45, CD69, CD80, CD86, CD90 or their ligands. A compound of Formula (I), (Ia), (II), (III), (IV), or (V) may also be combined with agents such as methotrexate, cyclosporine, FK506, rapamycin, mycophenolate mofetil, leflunomide, NSAIDs, for example, ibuprofen, corticosteroids such as prednisolone, phosphodiesterase inhibitors, adensosine agonists, antithrombotic agents, complement inhibitors, adrenergic agents, agents which interfere with signalling by proinflammatory cytokines such as TNF [symbol] or IL-1 (e.g. IRAK, NIK, IKK, p38 or MAP kinase inhibitors), IL-1β converting enzyme inhibitors, TACE inhibitors, T-cell signaling inhibitors such as kinase inhibitors, metalloproteinase inhibitors, sulfasalazine, azathioprine, 6-mercaptopurines, angiotensin converting enzyme inhibitors, soluble cytokine receptors and derivatives thereof (e.g. soluble p55 or p75 TNF receptors, sIL-1RI, sIL-1RII, sIL-6R) and antiinflammatory cytokines (e.g. IL-4, IL-10, IL-13 and TGFβ).

Preferred examples of therapeutic agents for multiple sclerosis in which a compound of Formula (I), (Ia), (II), (III), (IV), or (V) can be combined to include interferon-β, for example, IFNβ1a and IFNβ1b; copaxone, corticosteroids, caspase inhibitors, for example inhibitors of caspase-1, IL-1 inhibitors, TNF inhibitors, and antibodies to CD40 ligand and CD80.

A compound of Formula (I), (Ia), (II), (III), (IV), or (V) may also be combined with agents, such as alemtuzumab, dronabinol, daclizumab, mitoxantrone, xaliproden hydrochloride, fampridine, glatiramer acetate, natalizumab, sinnabidol, a-immunokine NNSO3, ABR-215062, AnergiX.MS, chemokine receptor antagonists, BBR-2778, calagualine, CPI-1189, LEM (liposome encapsulated mitoxantrone), THC.CBD (cannabinoid agonist), MBP-8298, mesopram (PDE4 inhibitor), MNA-715, anti-IL-6 receptor antibody, neurovax, pirfenidone allotrap 1258 (RDP-1258), sTNF-R1, talampanel, teriflunomide, TGF-beta2, tiplimotide, VLA-4 antagonists (for example, TR-14035, VLA4 Ultrahaler, Antegran-ELAN/Biogen), interferon gamma antagonists and IL-4 agonists.

Non-limiting examples of therapeutic agents for angina with which a compound of Formula (I), (Ia), (II), (III), (IV), or (V) of the invention can be combined include the following: aspirin, nitroglycerin, isosorbide mononitrate, metoprolol succinate, atenolol, metoprolol tartrate, amlodipine besylate, diltiazem hydrochloride, isosorbide dinitrate, clopidogrel bisulfate, nifedipine, atorvastatin calcium, potassium chloride, furosemide, simvastatin, verapamil HCl, digoxin, propranolol hydrochloride, carvedilol, lisinopril, spironolactone, hydrochlorothiazide, enalapril maleate, nadolol, ramipril, enoxaparin sodium, heparin sodium, valsartan, sotalol hydrochloride, fenofibrate, ezetimibe, bumetanide, losartan potassium, lisinopril/hydrochlorothiazide, felodipine, captopril and bisoprolol fumarate.

Non-limiting examples of therapeutic agents for ankylosing spondylitis with which a compound of Formula (I), (Ia), (II), (III), (IV), or (V) can be combined include the following: ibuprofen, diclofenac, misoprostol, naproxen, meloxicam, indomethacin, diclofenac, celecoxib, rofecoxib, sulfasalazine, methotrexate, azathioprine, minocyclin, prednisone, etanercept, and infliximab.

Non-limiting examples of therapeutic agents for asthma with which a compound of Formula (I), (Ia), (II), (III), (IV), or (V) can be combined include the following: albuterol, salmeterol/fluticasone, montelukast sodium, fluticasone propionate, budesonide, prednisone, salmeterol xinafoate, levalbuterol HCl, albuterol sulfate/ipratropium, prednisolone sodium phosphate, triamcinolone acetonide, beclomethasone dipropionate, ipratropium bromide, azithromycin, pirbuterol acetate, prednisolone, theophylline anhydrous, methylprednisolone sodium succinate, clarithromycin, zafirlukast, formoterol fumarate, influenza virus vaccine, amoxicillin trihydrate, flunisolide, allergy injection, cromolyn sodium, fexofenadine hydrochloride, flunisolide/menthol, amoxicillin/clavulanate, levofloxacin, inhaler assist device, guaifenesin, dexamethasone sodium phosphate, moxifloxacin HCl, doxycycline hyclate, guaifenesin/d-methorphan, p-ephedrine/cod/chlorphenir, gatifloxacin, cetirizine hydrochloride, mometasone furoate, salmeterol xinafoate, benzonatate, cephalexin, pe/hydrocodone/chlorphenir, cetirizine HCl/pseudoephed, phenylephrine/cod/promethazine, codeine/promethazine, cefprozil, dexamethasone, guaifenesin/pseudoephedrine, chlorpheniramine/hydrocodone, nedocromil sodium, terbutaline sulfate, epinephrine, methylprednisolone and metaproterenol sulfate.

Non-limiting examples of therapeutic agents for COPD with which a compound of Formula (I), (Ia), (II), (III), (IV), or (V) can be combined include the following: albuterol sulfate/ipratropium, ipratropium bromide, salmeterol/fluticasone, albuterol, salmeterol xinafoate, fluticasone propionate, prednisone, theophylline anhydrous, methylprednisolone sodium succinate, montelukast sodium, budesonide, formoterol fumarate, triamcinolone acetonide, levofloxacin, guaifenesin, azithromycin, beclomethasone dipropionate, levalbuterol HCl, flunisolide, ceftriaxone sodium, amoxicillin trihydrate, gatifloxacin, zafirlukast, amoxicillin/clavulanate, flunisolide/menthol, chlorpheniramine/hydrocodone, metaproterenol sulfate, methylprednisolone, mometasone furoate, p-ephedrine/cod/chlorphenir, pirbuterol acetate, p-ephedrine/loratadine, terbutaline sulfate, tiotropium bromide, (R,R)-formoterol, TgAAT, cilomilast and roflumilast.

Non-limiting examples of therapeutic agents for HCV with which a compound of Formula (I), (Ia), (II), (III), (IV), or (V) can be combined include the following: Interferon-alpha-2a, Interferon-alpha-2b, Interferon-alpha con1, Interferon-alpha-n 1, pegylated interferon-alpha-2a, pegylated interferon-alpha-2b, ribavirin, peginterferon alfa-2b+ribavirin, ursodeoxycholic acid, glycyrrhizic acid, thymalfasin, Maxamine, VX-497 and any compounds that are used to treat HCV through intervention with the following targets: HCV polymerase, HCV protease, HCV helicase, and HCV IRES (internal ribosome entry site).

Non-limiting examples of therapeutic agents for Idiopathic Pulmonary Fibrosis with which a compound of Formula (I), (Ia), (II), (III), (IV), or (V) can be combined include the following: prednisone, azathioprine, albuterol, colchicine, albuterol sulfate, digoxin, gamma interferon, methylprednisolone sod succ, lorazepam, furosemide, lisinopril, nitroglycerin, spironolactone, cyclophosphamide, ipratropium bromide, actinomycin d, alteplase, fluticasone propionate, levofloxacin, metaproterenol sulfate, morphine sulfate, oxycodone HCl, potassium chloride, triamcinolone acetonide, tacrolimus anhydrous, calcium, interferon-alpha, methotrexate, mycophenolate mofetil and interferon-gamma-10.

Non-limiting examples of therapeutic agents for myocardial infarction with which a compound of Formula (I), (Ia), (II), (III), (IV), or (V) can be combined include the following: aspirin, nitroglycerin, metoprolol tartrate, enoxaparin sodium, heparin sodium, clopidogrel bisulfate, carvedilol, atenolol, morphine sulfate, metoprolol succinate, warfarin sodium, lisinopril, isosorbide mononitrate, digoxin, furosemide, simvastatin, ramipril, tenecteplase, enalapril maleate, torsemide, retavase, losartan potassium, quinapril HCl/mag carb, bumetanide, alteplase, enalaprilat, amiodarone hydrochloride, tirofiban HCl m-hydrate, diltiazem hydrochloride, captopril, irbesartan, valsartan, propranolol hydrochloride, fosinopril sodium, lidocaine hydrochloride, eptifibatide, cefazolin sodium, atropine sulfate, aminocaproic acid, spironolactone, interferon, sotalol hydrochloride, potassium chloride, docusate sodium, dobutamine HCl, alprazolam, pravastatin sodium, atorvastatin calcium, midazolam hydrochloride, meperidine hydrochloride, isosorbide dinitrate, epinephrine, dopamine hydrochloride, bivalirudin, rosuvastatin, ezetimibe/simvastatin, avasimibe, and cariporide.

Non-limiting examples of therapeutic agents for psoriasis with which a compound of Formula (I), (Ia), (II), (III), (IV), or (V) can be combined include the following: calcipotriene, clobetasol propionate, triamcinolone acetonide, halobetasol propionate, tazarotene, methotrexate, fluocinonide, betamethasone diprop augmented, fluocinolone acetonide, acitretin, tar shampoo, betamethasone valerate, mometasone furoate, ketoconazole, pramoxine/fluocinolone, hydrocortisone valerate, flurandrenolide, urea, betamethasone, clobetasol propionate/emoll, fluticasone propionate, azithromycin, hydrocortisone, moisturizing formula, folic acid, desonide, pimecrolimus, coal tar, diflorasone diacetate, etanercept folate, lactic acid, methoxsalen, hc/bismuth subgal/znox/resor, methylprednisolone acetate, prednisone, sunscreen, halcinonide, salicylic acid, anthralin, clocortolone pivalate, coal extract, coal tar/salicylic acid, coal tar/salicylic acid/sulfur, desoximetasone, diazepam, emollient, fluocinonide/emollient, mineral oil/castor oil/na lact, mineral oil/peanut oil, petroleum/isopropyl myristate, psoralen, salicylic acid, soap/tribromsalan, thimerosal/boric acid, celecoxib, infliximab, cyclosporine, alefacept, efalizumab, tacrolimus, pimecrolimus, PUVA, UVB, and sulfasalazine.

Non-limiting examples of therapeutic agents for psoriatic arthritis with which a compound of Formula (I), (Ia), (II), (III), (IV), or (V) can be combined include the following: methotrexate, etanercept, rofecoxib, celecoxib, folic acid, sulfasalazine, naproxen, leflunomide, methylprednisolone acetate, indomethacin, hydroxychloroquine sulfate, prednisone, sulindac, betamethasone diprop augmented, infliximab, methotrexate, folate, triamcinolone acetonide, diclofenac, dimethylsulfoxide, piroxicam, diclofenac sodium, ketoprofen, meloxicam, methylprednisolone, nabumetone, tolmetin sodium, calcipotriene, cyclosporine, diclofenac sodium/misoprostol, fluocinonide, glucosamine sulfate, gold sodium thiomalate, hydrocodone bitartrate/apap, ibuprofen, risedronate sodium, sulfadiazine, thioguanine, valdecoxib, alefacept and efalizumab.

Non-limiting examples of therapeutic agents for restenosis with which a compound of Formula (I), (Ia), (II), (III), (IV), or (V) can be combined include the following: sirolimus, paclitaxel, everolimus, tacrolimus, ABT-578, and acetaminophen.

Non-limiting examples of therapeutic agents for sciatica with which a compound of Formula (I), (Ia), (II), (III), (IV), or (V) can be combined include the following: hydrocodone bitartrate/apap, rofecoxib, cyclobenzaprine HCl, methylprednisolone, naproxen, ibuprofen, oxycodone HCl/acetaminophen, celecoxib, valdecoxib, methylprednisolone acetate, prednisone, codeine phosphate/apap, tramadol hcl/acetaminophen, metaxalone, meloxicam, methocarbamol, lidocaine hydrochloride, diclofenac sodium, gabapentin, dexamethasone, carisoprodol, ketorolac tromethamine, indomethacin, acetaminophen, diazepam, nabumetone, oxycodone HCl, tizanidine HCl, diclofenac sodium/misoprostol, propoxyphene napsylate/apap, asa/oxycod/oxycodone ter, ibuprofen/hydrocodone bit, tramadol HCl, etodolac, propoxyphene HCl, amitriptyline HCl, carisoprodol/codeine phos/asa, morphine sulfate, multivitamins, naproxen sodium, orphenadrine citrate, and temazepam.

Preferred examples of therapeutic agents for SLE (Lupus) with which a compound of Formula (I), (Ia), (II), (III), (IV), or (V) can be combined include the following: NSAIDS, for example, diclofenac, naproxen, ibuprofen, piroxicam, indomethacin; COX2 inhibitors, for example, celecoxib, rofecoxib, valdecoxib; anti-malarials, for example, hydroxychloroquine; steroids, for example, prednisone, prednisolone, budenoside, dexamethasone; cytotoxics, for example, azathioprine, cyclophosphamide, mycophenolate mofetil, methotrexate; inhibitors of PDE4 or purine synthesis inhibitor, for example Cellcept®. A compound of Formula (I), (Ia), (II), (III), (IV), or (V) may also be combined with agents such as sulfasalazine, 5-aminosalicylic acid, olsalazine, Imuran® and agents which interfere with synthesis, production or action of proinflammatory cytokines such as IL-1, for example, caspase inhibitors like IL-1β converting enzyme inhibitors and IL-1ra. A compound of Formula (I), (Ia), (II), (III), (IV), or (V) may also be used with T cell signaling inhibitors, for example, tyrosine kinase inhibitors; or molecules that target T cell activation molecules, for example, CTLA-4-IgG or anti-B7 family antibodies, anti-PD-1 family antibodies. A compound of Formula (I), (Ia), (II), (III), (IV), or (V) can be combined with IL-11 or anti-cytokine antibodies, for example, fonotolizumab (anti-IFNg antibody), or anti-receptor receptor antibodies, for example, anti-IL-6 receptor antibody and antibodies to B-cell surface molecules. A compound of Formula (I), (Ia), (II), (III), (IV), or (V) may also be used with LJP 394 (abetimus), agents that deplete or inactivate B-cells, for example, Rituximab (anti-CD20 antibody), lymphostat-B (anti-BlyS antibody), TNF antagonists, for example, anti-TNF antibodies, D2E7 (PCT Publication No. WO 97/29131; HUMIRA™), CA2 (REMICADE™), CDP 571, TNFR-Ig constructs, (p75TNFRIgG (ENBREL™) and p55TNFRIgG (LENERCEPT™)).

In the compositions of the present invention the active compound may, if desired, be associated with other compatible pharmacologically active ingredients. For example, the compounds of this invention can be administered in combination with another therapeutic agent that is known to treat a disease or condition described herein. For example, with one or more additional pharmaceutical agents that inhibit or prevent the production of VEGF or angiopoietins, attenuate intracellular responses to VEGF or angiopoietins, block intracellular signal transduction, inhibit vascular hyperpermeability, reduce inflammation, or inhibit or prevent the formation of edema or neovascularization. The compounds of the invention can be administered prior to, subsequent to or simultaneously with the additional pharmaceutical agent, whichever course of administration is appropriate. The additional pharmaceutical agents include, but are not limited to, anti-edemic steroids, NSAIDS, ras inhibitors, anti-TNF agents, anti-IL1 agents, antihistamines, PAF-antagonists, COX-1 inhibitors, COX-2 inhibitors, NO synthase inhibitors, Akt/PTB inhibitors, IGF-1R inhibitors, PKC inhibitors, PI3 kinase inhibitors, calcineurin inhibitors and immunosuppressants. The compounds of the invention and the additional pharmaceutical agents act either additively or synergistically. Thus, the administration of such a combination of substances that inhibit angiogenesis, vascular hyperpermeability and/or inhibit the formation of edema can provide greater relief from the deletrious effects of a hyperproliferative disorder, angiogenesis, vascular hyperpermeability or edema than the administration of either substance alone. In the treatment of malignant disorders combinations with antiproliferative or cytotoxic chemotherapies or radiation are included in the scope of the present invention.

One or more compounds of the invention can be administered to a human patient by themselves or in pharmaceutical compositions where they are mixed with biologically suitable carriers or excipient(s) at doses to treat or ameliorate a disease or condition as described herein. Mixtures of these compounds can also be administered to the patient as a simple mixture or in suitable formulated pharmaceutical compositions. A therapeutically effective dose refers to that amount of the compound or compounds sufficient to result in the prevention or attenuation of a disease or condition as described herein. Techniques for formulation and administration of the compounds of the instant application may be found in references well known to one of ordinary skill in the art, such as “Remington's Pharmaceutical Sciences,” Mack Publishing Co., Easton, Pa., latest edition.

### Pharmaceutical Compositions and Modes of Administration

Suitable routes of administration may, for example, include oral, eyedrop, rectal, transmucosal, topical, or intestinal administration; parenteral delivery, including intramuscular, subcutaneous, intramedullary injections, as well as intrathecal, direct intraventricular, intravenous, intraperitoneal, intranasal, or intraocular injections.

Alternatively, one may administer the compound in a local rather than a systemic manner, for example, via injection of the compound directly into an edematous site, often in a depot or sustained release formulation.

Furthermore, one may administer the drug in a targeted drug delivery system, for example, in a liposome coated with endothelial cell-specific antibody.

The pharmaceutical compositions of the present invention may be manufactured in a manner that is itself known, e.g., by means of conventional mixing, dissolving, granulating, dragee-making, levigating, emulsifying, encapsulating, entrapping or lyophilizing processes.

Pharmaceutical compositions for use in accordance with the present invention thus may be formulated in a conventional manner using one or more physiologically acceptable carriers comprising excipients and auxiliaries which facilitate processing of the active compounds into preparations which can be used pharmaceutically. Proper formulation is dependent upon the route of administration chosen.

For injection, the agents of the invention may be formulated in aqueous solutions, preferably in physiologically compatible buffers such as Hanks' solution, Ringer's solution, or physiological saline buffer. For transmucosal administration, penetrants appropriate to the barrier to be permeated are used in the formulation. Such penetrants are generally known in the art.

For oral administration, the compounds can be formulated readily by combining the active compounds with pharmaceutically acceptable carriers well known in the art. Such carriers enable the compounds of the invention to be formulated as tablets, pills, dragees, capsules, liquids, gels, syrups, slurries, suspensions and the like, for oral ingestion by a patient to be treated. Pharmaceutical preparations for oral use can be obtained by combining the active compound with a solid excipient, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). If desired, disintegrating agents may be added, such as the cross-linked polyvinyl pyrrolidone, agar, or alginic acid or a salt thereof such as sodium alginate.

Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

Pharmaceutical preparations which can be used orally include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. All formulations for oral administration should be in dosages suitable for such administration.

For buccal administration, the compositions may take the form of tablets or lozenges formulated in conventional manner.

For administration by inhalation, the compounds for use according to the present invention are conveniently delivered in the form of an aerosol spray presentation from pressurized packs or a nebuliser, with the use of a suitable propellant, e.g., dichlorodifluoromethane, trichlorofluoromethane, dichlorotetrafluoroethane, carbon dioxide or other suitable gas. In the case of pressurized aerosol the dosage unit may be determined by providing a valve to deliver a metered amount. Capsules and cartridges of e.g. gelatin for use in an inhaler or insufflator may be formulated containing a powder mix of the compound and a suitable powder base such as lactose or starch.

The compounds can be formulated for parenteral administration by injection, e.g. bolus injection or continuous infusion. Formulations for injection may be presented in unit dosage form, e.g. in ampoules or in multi-dose containers, with an added preservative. The compositions may take such forms as suspensions, solutions or emulsions in oily or aqueous vehicles, and may contain formulatory agents such as suspending, stabilizing and/or dispersing agents.

Pharmaceutical formulations for parenteral administration include aqueous solutions of the active compounds in water-soluble form. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions.

Alternatively, the active ingredient may be in powder form for constitution with a suitable vehicle, e.g., sterile pyrogen-free water, before use.

The compounds may also be formulated in rectal compositions such as suppositories or retention enemas, e.g., containing conventional suppository bases such as cocoa butter or other glycerides.

In addition to the formulations described previously, the compounds may also be formulated as a depot preparation. Such long acting formulations may be administered by implantation (for example subcutaneously or intramuscularly or by intramuscular injection). Thus, for example, the compounds may be formulated with suitable polymeric or hydrophobic materials (for example as an emulsion in an acceptable oil) or ion exchange resins, or as sparingly soluble derivatives, for example, as a sparingly soluble salt.

An example of a pharmaceutical carrier for the hydrophobic compounds of the invention is a cosolvent system comprising benzyl alcohol, a nonpolar surfactant, a water-miscible organic polymer, and an aqueous phase. The cosolvent system may be the VPD co-solvent system. VPD is a solution of 3% w/v benzyl alcohol, 8% w/v of the nonpolar surfactant polysorbate 80, and 65% w/v polyethylene glycol 400, made up to volume in absolute ethanol. The VPD co-solvent system (VPD:5W) consists of VPD diluted 1:1 with a 5% dextrose in water solution. This co-solvent system dissolves hydrophobic compounds well, and itself produces low toxicity upon systemic administration. Naturally, the proportions of a co-solvent system may be varied considerably without destroying its solubility and toxicity characteristics. Furthermore, the identity of the co-solvent components may be varied: for example, other low-toxicity nonpolar surfactants may be used instead of polysorbate 80; the fraction size of polyethylene glycol may be varied; other biocompatible polymers may replace polyethylene glycol, e.g. polyvinyl pyrrolidone; and other sugars or polysaccharides may substitute for dextrose.

Alternatively, other delivery systems for hydrophobic pharmaceutical compounds may be employed. Liposomes and emulsions are well known examples of delivery vehicles or carriers for hydrophobic drugs. Certain organic solvents such as dimethysulfoxide also may be employed, although usually at the cost of greater toxicity. Additionally, the compounds may be delivered using a sustained-release system, such as semipermeable matrices of solid hydrophobic polymers containing the therapeutic agent. Various sustained-release materials have been established and are well known by those skilled in the art. Sustained-release capsules may, depending on their chemical nature, release the compounds for a few weeks up to over 100 days. Depending on the chemical nature and the biological stability of the therapeutic reagent, additional strategies for protein stabilization may be employed.

The pharmaceutical compositions also may comprise suitable solid or gel phase carriers or excipients. Examples of such carriers or excipients include but are not limited to calcium carbonate, calcium phosphate, various sugars, starches, cellulose derivatives, gelatin, and polymers such as polyethylene glycols.

Many of the compounds of the invention may be provided as salts with pharmaceutically compatible counterions. Pharmaceutically compatible salts may be formed with many acids, including but not limited to hydrochloric, sulfuric, acetic, lactic, tartaric, malic, succinic, etc. Salts tend to be more soluble in aqueous or other protonic solvents than are the corresponding free base forms.

Pharmaceutical compositions suitable for use in the present invention include compositions wherein the active ingredients are contained in an effective amount to achieve its intended purpose. More specifically, a therapeutically effective amount means an amount effective to prevent development of or to alleviate the existing symptoms of the subject being treated. Determination of the effective amounts is well within the capability of those skilled in the art.

### Dosage

For any compound used in a method of the present invention, the therapeutically effective dose can be estimated initially from cellular assays. For example, a dose can be formulated in cellular and animal models to achieve a circulating concentration range that includes the EC₅₀ as determined in cellular assays (i.e., the concentration of the test compound which achieves a half-maximal inhibition of a given receptor activity). In some cases it is appropriate to determine the EC₅₀ in the presence of 3 to 5% serum albumin since such a determination approximates the binding effects of plasma protein on the compound. Such information can be used to more accurately determine useful doses in humans. Further, advantageous compounds for systemic administration effectively modulate receptors of the S1P family in intact cells at levels that are safely achievable in plasma.

A therapeutically effective dose refers to that amount of the compound that results in amelioration of symptoms in a patient. Toxicity and therapeutic efficacy of such compounds can be determined by standard pharmaceutical procedures in cell cultures or experimental animals, e.g., for determining the maximum tolerated dose (MTD) and the ED₅₀ (effective dose for 50% maximal response). The dose ratio between toxic and therapeutic effects is the therapeutic index and it can be expressed as the ratio between MTD and ED₅₀. Compounds which exhibit high therapeutic indices are preferred. The data obtained from these cell culture assays and animal studies can be used in formulating a range of dosage for use in humans. The dosage of such compounds lies preferably within a range of circulating concentrations that include the ED₅₀ with little or no toxicity. The dosage may vary within this range depending upon the dosage form employed and the route of administration utilized. The exact formulation, route of administration and dosage can be chosen by the individual physician in view of the patient's condition. (See e.g. Fingl et al., 1975, in “The Pharmacological Basis of Therapeutics”, Ch. 1 p 1). In the treatment of crises, the administration of an acute bolus or an infusion approaching the MTD may be advantageous to obtain a rapid response.

Dosage amount and interval may be adjusted individually to provide plasma levels of the active moiety which are sufficient to modulate receptors of the S1P family, or minimal effective concentration (MEC). The MEC will vary for each compound but can be estimated from in vitro data; e.g. the concentration necessary to achieve 50-90% inhibition of binding of the natural ligand using the assays described herein. Dosages necessary to achieve the MEC will depend on individual characteristics and route of administration. However, HPLC assays or bioassays can be used to determine plasma concentrations.

Dosage intervals can also be determined using the MEC value. Compounds should be administered using a regimen which maintains plasma levels above the MEC for 10-90% of the time, preferably between 30-90% and more preferably between 50-90% until the desired amelioration of symptoms is achieved. In cases of local administration or selective uptake, the effective local concentration of the drug may not be related to plasma concentration. The amount of composition administered will, of course, be dependent on the subject being treated, on the subject's weight, the severity of the affliction, the manner of administration and the judgment of the prescribing physician.

The compositions may, if desired, be presented in a pack or dispenser device which may contain one or more unit dosage forms containing the active ingredient. The pack may for example comprise metal or plastic foil, such as a blister pack. The pack or dispenser device may be accompanied by instructions for administration. Compositions comprising a compound of the invention formulated in a compatible pharmaceutical carrier may also be prepared, placed in an appropriate container, and labeled for treatment of an indicated condition.

### Exemplary Formulations

In some formulations it may be beneficial to use the compounds of the present invention in the form of particles of very small size, for example as obtained by fluid energy milling.

The use of compounds of the present invention in the manufacture of pharmaceutical compositions is illustrated by the following description. In this description the term “active compound” denotes any compound of the invention but particularly any compound which is the final product of one of the preceding Examples.

### a) Capsules

In the preparation of capsules, 10 parts by weight of active compound and 240 parts by weight of lactose can be de-aggregated and blended. The mixture can be filled into hard gelatin capsules, each capsule containing a unit dose or part of a unit dose of active compound.

### b) Tablets

Tablets can be prepared, for example, from the following ingredients:

| | Parts by weight |
|---|---|
| Active compound | 10 |
| Lactose | 190 |
| Maize starch | 22 |
| Polyvinylpyrrolidone | 10 |
| Magnesium stearate | 3 |

The active compound, the lactose and some of the starch can be de-aggregated, blended and the resulting mixture can be granulated with a solution of the polyvinylpyrrolidone in ethanol. The dry granulate can be blended with the magnesium stearate and the rest of the starch. The mixture is then compressed in a tabletting machine to give tablets each containing a unit dose or a part of a unit dose of active compound.

### c) Enteric Coated Tablets

Tablets can be prepared by the method described in (b) above. The tablets can be enteric coated in a conventional manner using a solution of 20% cellulose acetate phthalate and 3% diethyl phthalate in ethanol:dichloromethane (1:1).

### d) Suppositories

In the preparation of suppositories, for example, 100 parts by weight of active compound can be incorporated in 1300 parts by weight of triglyceride suppository base and the mixture formed into suppositories each containing a therapeutically effective amount of active ingredient.

The present invention also comprises the use of a compound of Formula (I), (Ia), (II), (III), (IV), or (V) as a medicament.

A further aspect of the present invention provides the use of a compound of Formula (I), (Ia), (II), (III), (IV), or (V) or a salt thereof in the manufacture of a medicament for treating vascular hyperpermeability, angiogenesis-dependent disorders, proliferative diseases and/or disorders of the immune system in mammals, particularly human beings.

The present invention also provides a method of treating vascular hyperpermeability, inappropriate neovascularization, proliferative diseases and/or disorders of the immune system which comprises the administration of a therapeutically effective amount of a compound of Formula (I), (Ia), (II), (III), (IV), or (V) to a mammal, particularly a human being, in need thereof.

The teachings of all references, including journal articles, patents and published patent applications, are incorporated herein by reference in their entirety.

### S1P Receptor GTPγS Assays

The [³⁵S]GTPγS binding assay can be run using both scintillation proximity assay (SPA) and filtration methods. Both formats are in 96 well plates and utilize membranes from a stable or transient CHO human cell lines overexpressing S1P₁, S1P₃, S1 P₄ or S1P₅. Compound stocks were made up to 10 mM using DMSO and serial dilutions were carried out using 100% DMSO. Compounds were transferred to 96 well plates to yield a final DMSO concentration of 1% for all assays (1 ul for a 100 μl assay volume). Frozen membranes were thawed and diluted in assay buffer containing of 20 mM HEPES pH 7.4, 0.1% fatty acid-free BSA, 100 mM NaCl, 5 mM MgCl₂ and 10 μM GDP. For the SPA assay membranes are premixed with WGA-SPA beads to yield a final concentration per well of 5 μg membrane and 500 μg of bead. For the filtration assay, membranes are added directly to the incubation plate at 5 ug per well. The assay begins with the addition of 50 μl of the membrane or membrane/bead mixture to each well of the assay plate. Next, 50 μl of 0.4 nM [³⁵S]GTPγS is added to each well and incubated for 30 minutes. Nonspecific binding is measured using 10 μM unlabeled GTPγS. For the SPA, assay the plates are spun and then read on the Topcount. For the filtration assay the plate is harvested onto GF-C filtration plates using a Packard 96 well harvester.

### Inhibition of [³³P]S1P Binding to S1P Receptors

Radio ligand binding was carried out using membranes from transiently transfected HEK cells overexpressing S1P₁, S1P₃, S1P₄ or S1P₅. All compounds are dissolved in DMSO and serial dilutions were carried out in DMSO prior to addition to assay buffer. Final assay DMSO concentrations are 1% (v/v). [³³P]S1P is purchased from Perkin Elmer and used at 50 pM in all assays. Frozen membranes are thawed and resuspended in assay buffer containing 50 mM HEPES pH 7.4, 100 mM NaCl, 10 mM MgCl₂ and 0.1% fatty acid free BSA. Membrane is added to give 5-10 μg of membrane per well. Non-specific binding is determined in the presence of cold 1 μM S1P. Incubations are carried out at room temperature for 45-60 minutes before filtering onto GF/C filtration plates using a Packard 96 well harvester. Plates are dried before adding Microscint to each well, sealed and counted on a Topcount.

### Abbreviations

ACN Acetonitrile
Binap 2,2′-bis(diphenylphosphino)-1,1′-binaphthyl
CH₂Cl₂ dichloromethane
CHCl₃ Chloroform
CO₂ Carbon dioxide
DAST Diethylaminosulfur trifluoride
DBAD Di-tert-butyl azodicarboxylate
DBU 1,8-Diazabicyclo(5.4.0)undec-7-ene
DCC N,N′-Dicyclohexylcarbodiimide
DCE Dicholorethane
DCM Dichloromethane
DIAD Diisopropyl azodicarboxylate
Dibal-H Diisobutylaluminum hydride
DIC N,N′-Diisopropylcarbodiimide
DIEA N,N-Diisopropylethylamine
DMA N,N-Dimethylacetamide
DME 1,2-dimethoxyethane
DMF N,N-Dimethylformamide
DMSO Dimethyl sulfoxide
EA Ethyl acetate
EDC 1,2-dichloroethane
EDCI 1-(3-Dimethylaminopropyl)-3-ethylcarbodiimide
EtOH Ethanol
EtOAc Ethyl acetate
Et₃N Triethylamine
FCC Flash column chromatography
h hour(s)
HBTU O-Benzotriazol-1-yl-N,N,N′,N′-tetramethyluronium Hexafluorophosaphate
HATU O-(7-Azabenzotriazol-1-yl)-N,N,N′,N′-tetramethyluronium Hexafluorophosaphate
HCl Hydrochloric acid
HOBt 1-Hydroxybenzotriazole
HOAT 1-Hydroxy-7-azabenzotriazole
HPLC High Performance Liquid Chromatography
KHMDS Potassium hexamethyldisilazide
MeOH Methanol

### Min Minutes

MW Microwave
NaOH Sodium hydroxide
NIS N-iodosuccinimide
NMP 1-methyl-2-pyrrolidone
PS-DCC Polymer-supported carbodiimide
PS-PPh₃ Polymer-supported triphenylphosphine
RBF Round bottom flask
RP Reverse Phase
Rₜ Retention time
RT Room temperature
THF Tetrahydrofuran
i-PrOH 2-Propanol
PPh₃ Triphenylphosphine
SFC Supercritical fluid chromatography
SOCl₂ Thionyl chloride

### Analytical Methods

Analytical data is defined either within the general procedures or in the tables of examples. Unless otherwise stated, all ¹H or ¹³C NMR data were collected on a Varian Mercury Plus 400 MHz or a Bruker DRX 400 MHz instrument; chemical shifts are quoted in parts per million (ppm). High-pressure liquid chromatography (HPLC) analytical data are either detailed within the experimental or referenced to the table of HPLC conditions, using the lower case method letter, in Table 1.

TABLE 1
List of HPLC methods

| | HPLC Conditions |
|---|---|
| | Unless indicated otherwise, mobile phase A was 10 mM ammonium acetate, mobile |
| Method | phase B was HPLC grade ACN. |
| a | 5-95% B over 3.7 min with a hold at 95% B for 1 min (1.3 mL/min flow rate). 4.6 × |
| | 50 mm Zorbax XDB C18 column (5 μm particles). Detection methods are diode |
| | array (DAD) and evaporative light scattering (ELSD) detection as well as pos/neg |
| | electrospray ionization. |
| b | 5-60% B over 1.5 min then 60-95% B to 2.5 min with a hold at 95% B for 1.2 min |
| | (1.3 mL/min flow rate). 4.6 × 30 mm Vydac Genesis C8 column (4 μm particles). |
| | Detection methods are diode array (DAD) and evaporative light scattering (ELSD) |
| | detection as well as pos/neg electrospray ionization. |
| c | 30-95% B over 2.0 min with a hold at 95% B for 1.7 min (1.0 mL/min flow rate). 4.6 × |
| | 30 mm Vydac Genesis C8 column (4 μm particles). Detection methods are diode |
| | array (DAD) and evaporative light scattering (ELSD) detection as well as pos/neg |
| | electrospray ionization. |
| d | 30-95% B over 2.0 min with a hold at 95% B for 1.5 min (1.0 mL/min flow rate). |
| | UV λ = 210-360 nm; Genesis C8, 4 μm, 30 × 4.6 mm column; ESI +ve/−ve) |
| e | A gradient of 10-100% ACN (B) and 0.1% trifluoroacetic acid in water (A) is used, |
| | at a flow rate of 1.5 mL/min (0-0.1 min 10% A, 0.1-3.1 min 10-100% B, 3.1-3.9 min |
| | 100-10% B, 3.9-4.0 min 100-10% B). 2.1 mm × 30 mm Phenomenex Luna Combi- |
| | HTS C8 (5 μm particles). Detection methods are diode array (DAD) and evaporative |
| | light scattering (ELSD) detection as well as APCI ionization. |
| f | Gradient was 5-35% B in 4 min then 35-95% B to 6 min with a hold at 95% B for |
| | 1.7 min (1.3 mL/min flow rate). Mobile phase A was water with 0.1% formic acid, |
| | mobile phase B was HPLC grade ACN. The column used for the chromatography |
| | was a 4.6 × 30 mm Vydac Genesis C8 column (4 μm particles). Detection methods |
| | are diode array (DAD) and evaporative light scattering (ELSD) detection as well as |
| | pos/neg electrospray ionization. |
| g | The gradient was 5-60% B in 1.5 min then 60-95% B to 2.5 min with a hold at 95% |
| | B for 1.2 min (1.3 mL/min flow rate). Mobile phase A was 10 mM ammonium |
| | acetate, mobile phase B was HPLC grade ACN. The column used for the |
| | chromatography is a 4.6 × 50 mm MAC-MOD Halo C18 column (2.7 μm particles). |
| | Detection methods are diode array (DAD) and evaporative light scattering (ELSD) |
| | detection as well as positive/negative electrospray ionization. |
| h | The gradient was 5-60% B in 1.5 min then 60-95% B to 2.5 min with a hold at 95% |
| | B for 1.2 min (1.3 mL/min flow rate). Mobile phase A was 10 mM ammonium |
| | acetate, mobile phase B was HPLC grade ACN. The column used for the |
| | chromatography was a 4.6 × 50 mm MAC-MOD Halo C8 column (4 μm particles). |
| | Detection methods are diode array (DAD) and evaporative light scattering (ELSD) |
| | detection as well as positive/negative atmospheric pressure chemical ionization |
| | (APCI). |
| i | The gradient was 30-60% B in 1.50 min then 60-95% B to 2.5 min with a hold at |
| | 95% B for 1.2 min (1.3 mL/min flow rate). Mobile phase A was 10 mM ammonium |
| | acetate, mobile phase B was HPLC grade ACN. The column used for the |
| | chromatography is a 4.6 × 50 mm MAC-MOD Halo C8 column (2.7 μm particles). |
| | Detection methods are diode array (DAD) and evaporative light scattering (ELSD) |
| | detection as well as positive/negative electrospray ionization. |
| j | The gradient was 5-60% B in 1.5 min then 60-95% B to 2.5 min with a hold at 95% |
| | B for 1.2 min (1.3 mL/min flow rate). Mobile phase A was 10 mM ammonium |
| | acetate, mobile phase B was HPLC grade ACN. The column used for the |
| | chromatography is a 4.6 × 50 mm MAC-MOD Halo C8 column (2.7 μm particles). |
| | Detection methods are diode array (DAD) and evaporative light scattering (ELSD) |
| | detection as well as positive/negative electrospray ionization. |
| k | The gradient was 5-60% B in 0.75 min then 60-95% B to 1.15 min with a hold at |
| | 95% B for 0.75 min (1.3 mL/min flow rate). Mobile phase A was 10 mM ammonium |
| | acetate, mobile phase B was HPLC grade ACN. The column used for the |
| | chromatography is a 4.6 × 50 mm MAC-MOD Halo C8 column (2.7 μm particles). |
| | Detection methods are diode array (DAD) and evaporative light scattering (ELSD) |
| | detection as well as positive/negative electrospray ionization. |
| l | Samples were purified by preparative HPLC on a Phenomenex Luna C8(2) 5 um |
| | 100 Å AXIA column (30 mm × 75 mm). A gradient of ACN (A) and 0.1% |
| | trifluoroacetic acid in water (B) was used, at a flow rate of 50 mL/min (0-0.5 min |
| | 10% A, 0.5-6.0 min linear gradient 10-100% A, 6.0-7.0 min 100% A, 7.0-8.0 min |
| | linear gradient 100-10% A). Samples were injected in 1.5 mL DMSO:MeOH (1:1). |
| m | A gradient of 10-100% ACN (A) and 0.1% trifluoroacetic acid in water (B) was |
| | used, at a flow rate of 2.0 mL/min (0-0.1 min 10% A, 0.1-2.6 min 10-100% A, 2.6-2.9 min |
| | 100% A, 2.9-3.0 min 100-10% A. 0.5 min post-run delay). |
| n | 9 minute chromatogram. 5_95 NH4OAc 8 m GC8 - The column used for the |
| | chromatography was a 4.6 × 30 mm Vydac Genesis C8 column (4 mm particles). The |
| | gradient was 5-35% B in 4 min then 35-95% B to 6 min with a hold at 95% B for 1.7 min |
| | (1.3 mL/min flow rate). Mobile phase A was 10 mM ammonium acetate, |
| | mobile phase B was HPLC grade ACN. Detection methods are diode array (DAD) |
| | and evaporative light scattering (ELSD) detection as well as pos/neg electrospray |
| | ionization. |
| o | 6 minute chromatogram non polar 6 min GC8: The column used for the |
| | chromatography was a 4.6 × 30 mm Vydac Genesis C8 column (4 mm particles). The |
| | gradient was 30-95% B in 2 min then hold at 95% B to 5.7 min (1.3 mL/min flow |
| | rate). Mobile phase A was 10 mM ammonium acetate, mobile phase B was HPLC |
| | grade ACN. Detection methods are diode array (DAD) and evaporative light |
| | scattering (ELSD) detection as well as pos/neg electrospray ionization. |
| p | 5 minute chromatogram Purity QC method: The column used for the |
| | chromatography was a 4.6 × 50 mm Zorbax XDB C18 column (5 mm particles). The |
| | gradient was 5-95% B in 3.7 min with a hold at 95% B for 1 min (1.3 mL/min flow |
| | rate). Mobile phase A was 10 mM ammonium acetate, mobile phase B was HPLC |
| | grade ACN. Detection methods are diode array (DAD) and evaporative light |
| | scattering (ELSD) detection as well as pos/neg electrospray ionization. |
| q | HPLC Method: 80:20 0.1% H₃PO₄/ACN to 30:70% in 15 min then to 5:95 in 3 min, |
| | hold for 4 min then increase to 80:20 in 0.1 min, keep at 80:20 for 5 min. Column |
| | Zorbax Eclipse XDB C18, 150 × 4.6 mm, 3.5 micron, stop time 27 min, flow rate 1.5 mL/min, |
| | column temperature 25° C. |
| r | HPLC 80:20 0.1% H3PO4/ACN to 30:70% in 15 min then to 5:95 in 3 min, keep for |
| | 4 min then increase to 80:20 in 0.1 min, keep at 80:20 for 5 min. Column Zorbax |
| | Eclipse XDB C18, 150 × 4.6 mm, 3.5 micron, stop time 27 min, flow rate 1.5 mL/min, |
| | column temperature 25° C. |

### Purification Methods

For the general procedures, the final compounds may be purified by any technique or combination of techniques known to one skilled in the art. Some examples that are not limiting include flash chromatography with a solid phase (e.g. silica gel, alumina, etc.) and a solvent (or combination of solvents) that elutes the desired compounds (e.g. heptane, EtOAc, DCM, MeOH, MeCN, water, etc.); preparatory TLC with a solid phase (e.g. silica gel, alumina etc.) and a solvent (or combination of solvents) that elutes the desired compounds (e.g. heptane, EtOAc, DCM, MeOH, MeCN, water, etc.); reverse phase HPLC (see Table 1 for some non-limiting conditions); recrystallization from an appropriate solvent (e.g. MeOH, EtOH, IPA, EtOAc, toluene, etc.) or combination of solvents (e.g. EtOAc/heptane, EtOAc/MeOH, etc.); chiral LC with a solid phase and an appropriate solvent (see Table 2 for some non-limiting conditions) to elute the desired compound; chiral SFC with a solid phase and CO₂ with an appropriate modifier (e.g. MeOH, EtOH, IPA with or without additional modifier such as diethylamine, TFA, etc.); precipitation from a combination of solvents (e.g. DMF/water, DMSO/DCM, EtOAc/heptane, etc.); trituration with an appropriate solvent (e.g. EtOAc, DCM, MeCN, MeOH, EtOH, IPA, n-IPA, etc.); extractions by dissolving a compound in a liquid and washing with an appropriately immiscible liquid (e.g. DCM/water, EtOAc/water, DCM/saturated aqueous NaHCO₃, EtOAc/saturated aqueous NaHCO₃, DCM/10% aqueous HCl, EtOAc/10% aqueous HCl, etc.); distillation (e.g. simple, fractional, Kugelrohr, etc.); gas chromatography using an appropriate temperature, carrier gas and flow rate; sublimation at an appropriate temperature and pressure; filtration through a media (e.g. Florosil®, alumina, Celite®, silica gel, etc.) with a solvent (e.g. heptane, hexanes, EtOAc, DCM, MeOH, etc.) or combination of solvents; salt formation with solid support (resin based, e.g. ion exchange) or without. Some descriptions of these techniques can be found in the following references: Gordon, A. J. and Ford, R. A. “The Chemist's Companion”, 1972; Palleros, D. R. “Experimental Organic Chemistry”, 2000; Still, W. C., Kahn and M. Mitra, A. J. Org. Chem. 1978, 43, 2923; Yan, B. “Analysis and Purification Methods in Combinatorial Chemistry”, 2003; Harwood, L. M., Moody, C. J. and Percy, J. M. “Experimental Organic Chemistry: Standard and Microscale, 2^{nd} Edition”, 1999; Stichlmair, J. G. and Fair, J. R. “Distillation; Principles and Practices”, 1998; Beesley, T. E. and Scott, R. P. W. “Chiral Chromatography”, 1999; Landgrebe, J. A. “Theory and Practice in the Organic Laboratory, 4^{th} Ed.”, 1993; Skoog, D. A. and Leary, J. J. “Principles of Instrumental Analysis, 4^{th} Ed.”, 1992; G. Subramanian, “Chiral Separation Techniques, 3^{rd} Edition”, 2007; Y. Kazakevich, R. Lobrutto, “HPLC for Pharmaceutical Scientists”, 2007.

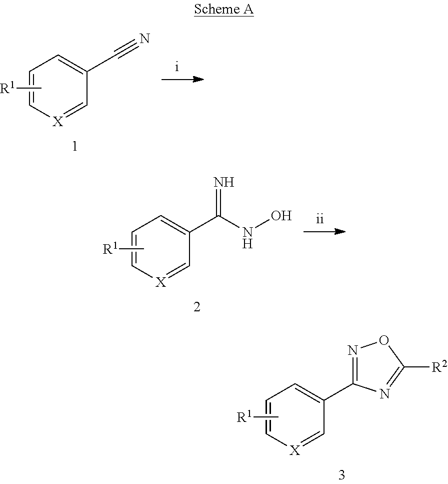

A method for preparing di-substituted oxadiazole compounds of the invention is illustrated in Scheme A (X═CR³ or N). In Scheme A, step i, a suitably substituted nitrile compound 1 (commercially available or made through General procedure A or B) is reacted with hydroxylamine to give compound 2. These types of reactions are well established in the literature (see, for example, Yan, et al., Bioorg & Med Chem Lett 2006, 16(14), 3679-3683). This reaction is typically conducted in a protic solvent (such as MeOH or EtOH) at temperatures at or below reflux (such as 60° C.). The product 2 is typically isolated from the reaction mixture as a solid by concentrating the mixture. Compound 2 can be used as it is. Coupling of compound 2 with a suitable acid or acid chloride followed by ring closure to produce compound 3 is shown in step ii. The coupling reaction is typically carried out with carboxylic acids in the presence of a coupling reagent (such as HOBt, DCC) or with acid chlorides in the presence of an organic base (such as DIEA, Et₃N) at room temperature or elevated temperature (for example, 20-180° C.) in a solvent such as DMF or DMA. The subsequent ring closure reaction is complete in situ at elevated temperature (for example 160° C.) (see, for example, Wang, et al., Org Lett 2005 7(5), 925-928). The compounds 3 can then be isolated and purified using standard techniques (such as reverse-phase liquid chromatography or SFC).

### General Synthetic Schemes

The general synthetic schemes that were utilized to construct the majority of compounds disclosed in this application are described below in (Schemes 1-3).

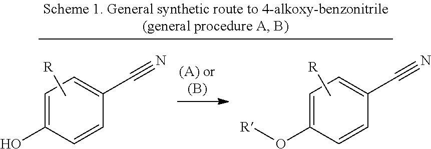

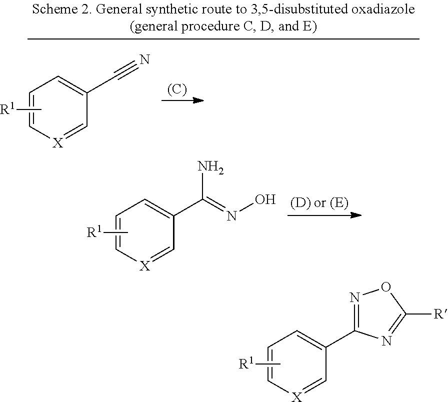

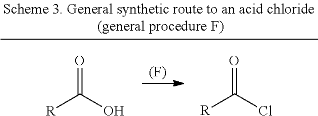

### List of General Procedures

General Procedure A: Preparation of 4-alkoxy-benzonitrile using triphenylphosphine
General Procedure B: Preparation of 4-alkoxy-benzonitrile using polymer-bound triphenylphosphine
General Procedure C: Preparation of hydroxyamidine
General Procedure D: Oxadiazole formation from an acid
General Procedure E: Oxadiazole formation from an acid chloride
General Procedure F: Formation of an acid chloride from a carboxylic acid
General Procedure G: Formation of aldehyde from nitrile
General Procedure H: Amination of aldehyde
General Procedure I: Alkylation of indole with acrylate
General Procedure J: Alkylation of indole with an alkyl halide
General Procedure K: Deprotection of tert-butyl ester or ether
General Procedure L: Amination of aryl halide
General Procedure M: Alkylation of phenol with an alkyl halide
General Procedure N: Debenzylation
General Procedure O: Deprotection of protected 1,2-diol
General Procedure P: Nucleophilic displacement of an aryl fluoride
General Procedure Q: Hydrolysis of an ester
General Procedure R: Mitsunobu coupling to a phenol
General Procedure S: Deprotection of phosphonate ester with or without bromination
General Procedure T: Preparation of benzylic olefin from ketone
General Procedure U: Reduction of benzylic olefin
General Procedure V: Hydrolysis of ester
General Procedure W: Alkylation of a phenol

### Example of Use of General Procedures

The general procedure letter codes constitute a synthetic route to the final product. A worked example of how the route is determined is given below using Example A.33 as a non-limiting illustration. Example A.33, 4-[3-(3-chloro-4-isopropoxy-phenyl)-[1,2,4]oxadiazol-5-yl]-pyridine was prepared from 3-chloro-N-hydroxy-4-isopropoxy-benzamidine using general procedure D, as represented in the following synthetic scheme:

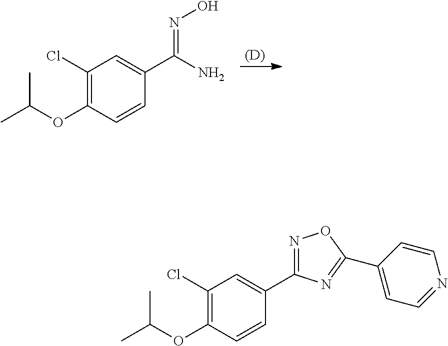

The precursor to Example A.33, 3-chloro-N-hydroxy-4-isopropoxy-benzamidine was prepared using the route (A, C). This translates into the following synthetic sequence, where the hydroxyamidine starting material used in general procedure D is the product by the following the procedure A and C, in the given order.

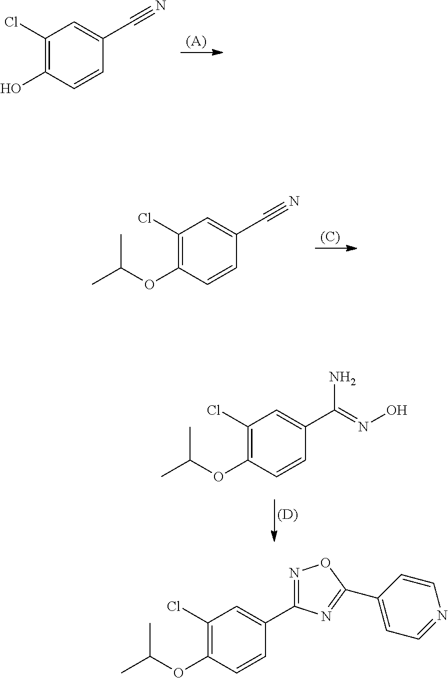

### PREPARATIONS AND EXAMPLES

The general synthetic methods used in each General Procedure follow and include an illustration of a compound that was synthesized using the designated General Procedure. None of the specific conditions and reagents noted herein are to be construed as limiting the scope of the invention and are provided for illustrative purposes only. All starting materials are commercially available from Sigma-Aldrich (including Fluka and Discovery CPR) unless otherwise noted after the chemical name. Reagent/reactant names given are as named on the commercial bottle or as generated by IUPAC conventions, CambridgeSoft® ChemDraw Ultra 9.0.7, CambridgeSoft® Chemistry E-Notebook 9.0.127, or AutoNom 2000.

### General Procedure A

### Preparation of 4-alkoxy-benzonitrile Using Triphenylphosphine

Triphenylphosphine (1-3 equivalents, preferably 1.6 equivalents) and 4-hydroxy-benzonitrile (1-3 equivalents, preferably 1 equivalent) are dissolved in an anhydrous organic solvent such as dichloromethane, toluene, or tetrahydrofuran (preferably tetrahydrofuran) under an atmosphere of nitrogen. After a brief stirring, an azodicarboxylate such as diethyl azodicarboxylate, diisopropyl azodicarboxylate, or di-tert-butyl azodicarboxylate (preferably di-tert-butyl azodicarboxylate) (1-3 equivalents, preferably 1.6 equivalents) is added to the solution and the mixture is stirred for a few minutes before addition of an anhydrous alcohol (1-3 equivalents, preferably 1.25 equivalents). The reaction mixture is stirred at 0-100° C. (preferably about 23° C.) under an atmosphere of nitrogen for a period of about 2-24 hours (preferably 16 hours). The solvent is removed under reduced pressure. The crude product can be further purified by flash column chromatography.

### Exemplification of General Procedure A

### Preparation of 3-chloro-4-isopropoxy-benzonitrile

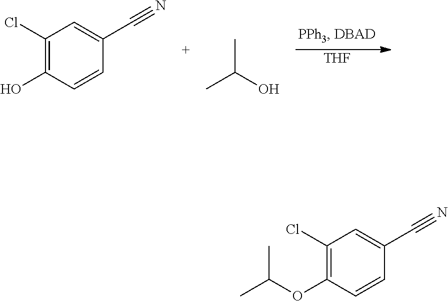

Into a round bottom flask containing triphenylphosphine (27.3 g, 104 mmol) and 3-chloro-4-hydroxy-benzonitrile (10 g, 65 mmol) was added anhydrous tetrahydrofuran (600 mL). The mixture was stirred briefly under nitrogen, di-tert-butyl azodicarboxylate (24 g, 104 mmol) was then added. The mixture was stirred for a few minutes, followed by addition of anhydrous isopropanol (6.23 mL, 81.4 mmol). The reaction mixture was stirred at room temperature overnight under nitrogen. The crude product was purified by flash chromatography using 1:4 (v/v) ethyl acetate/heptane as eluent. Fractions were dried to give 3-chloro-4-isopropoxy-benzonitrile (12.2 g, 91%) as a red-orange semi-solid.

LC/MS (Table 1, Method d) Rₜ=2.36 min, m/z 152.1 (M+H)⁺; ¹H NMR (400 MHz, DMSO-d₆) δ 7.74 (d, 1H), 7.61 (dd, 1H), 7.14 (d, 1H), 4.75 (sept., 1H), 1.34 (d, 6H)

### General Procedure B

### Preparation of 4-alkoxy-benzonitrile Using Polymer-Bound Triphenylphosphine

To an alcohol (1-3 equivalents, preferably 1 equivalent) and 4-hydroxy-benzonitrile (1-3 equivalents, preferably 1 equivalent) dissolved in a suitable solvent such as dichloromethane, dichloroethane, tetrahydrofuran, or 1,4-dioxane (preferable tetrahydrofuran) is added polymer-bound triphenylphosphine (1-3 equivalents, preferably 2 equivalents) and an azodicarboxylate such as diethyl azodicarboxylate, DIAD, or di-tert-butyl azodicarboxylate (preferably DIAD) (1-2 equivalents, preferably 1.5 equivalents). The mixture is shaken at about 0-100° C. (preferably about 23° C.) for a period of 4-24 hours (preferably 16 hours). The crude mixture is filtered and the resin is washed with a suitable solvent such as dichloromethane, dichloroethane, THF, or 1,4-dioxane (preferably THF). The filtrate is concentrated to dryness under reduced pressure and the residue is subjected to General Procedure C.

### Exemplification of General Procedure B

### Preparation of 3-chloro-4-(1-ethyl-propoxy)-benzonitrile

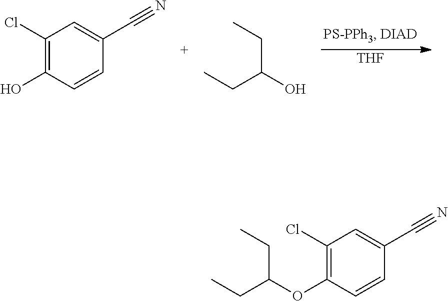

Into a scintillation vial containing a solution of pentan-3-ol (22 mg, 0.25 mmol) dissolved in THF (2 mL) was added a solution of 3-chloro-4-hydroxy-benzonitrile (38 mg, 0.25 mmol) in THF (2 mL) followed by PS—PPh₃ resin (357 mg, 0.5 mmol, loading 1.4 mmol/g) and a solution of DIAD (76 mg, 0.375 mmol) in THF (2 mL). The vial was capped and shaken at room temperature overnight. The reaction mixture was filtered and the resin was washed with THF (4 mL). The filtrate was concentrated to dryness to give 3-chloro-4-(1-ethyl-propoxy)-benzonitrile.

### General Procedure C

### Preparation of Hydroxyamidine

To a solution of benzonitrile (1-3 equivalents, preferably 1 equivalent) in a suitable solvent such as methanol, ethanol, isopropanol, or water (preferably ethanol) is added (1-50 equivalents, preferably 1.1 equivalents). The reaction mixture is heated at about 25-100° C. (preferably 60° C.) for a period of about 2-24 hours (preferably 16 hours). The solvents are removed under reduced pressure. The crude product is dried under vacuum and then subjected to General Procedure D or E.

### Exemplification of General Procedure C

### Preparation of 3-chloro-N-hydroxy-4-isopropoxy-benzamidine

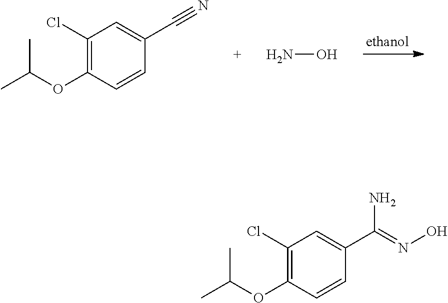

Into a round bottom flask was added 3-chloro-4-isopropoxy-benzonitrile (5.00 g, 25.6 mmol), hydroxylamine (50% by weight in water, 1.86 mL, 28.1 mmol) and ethanol (150 mL). The mixture was heated at about 60° C. overnight. Upon completion of the reaction, the mixture was concentrated to dryness under reduced pressure to give 3-chloro-N-hydroxy-4-isopropoxy-benzamidine (5.76 g, 94%) as a light yellow solid.

LC/MS (Table 1, Method a) Rₜ=2.09 min, m/z 229 (M+H)⁺; ¹H NMR (400 MHz, DMSO-d₆) δ 9.58 (s, 1H), 7.70 (d, 1H), 7.59 (dd, 1H), 7.15 (d, 1H), 5.81 (s, 2H), 4.69 (sept., 1H), 1.29 (d, 6H)

### General Procedure D

### Oxadiazole Formation from an Acid

To a reaction vial is added a hydroxyamidine (0.9-1.5 equivalents, preferably 1.1 equivalent), an acid (0.9-1.5 equivalents, preferably 1 equivalent), a coupling reagent such as HBTU, HATU, HOBt, or polymer-bound HOBt (preferably HOBt) (1-2 equivalents, preferably 1 equivalent), a carbodiimide such as PS, EDCI, DIC, DCC or polymer-bound DCC (preferably polymer-bound DCC) (1.5-3 equivalents, preferably 3 equivalents), a base such as diisopropylethylamine, triethylamine, or N-methylmorpholine (preferably diisopropylamine) (1-3 equivalents, preferably 3 equivalents) and a suitable solvent such as DMF, DMA, or ACN (preferably ACN). The reaction vial is capped and heated (conventional heating or microwave heating, preferably microwave heating) at 100-200° C. (preferably 160° C.) for a period of 15-45 min (preferably 30 min) After cooling down to RT, the crude reaction mixture is filtered, washed with a suitable solvent such as DMF, DMA, or ACN (preferably ACN), and the filtrate is concentrated to dryness under reduced pressure. The crude product is further purified by chromatography.

### Exemplification of General Procedure D

### Preparation of 4-[3-(3-chloro-4-isopropoxy-phenyl)-[1,2,4]oxadiazol-5-yl]-3-methyl-pyridine

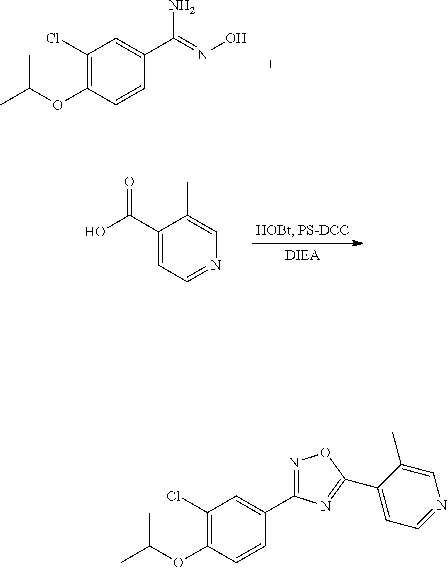

To a microwave vial charged with 3-chloro-N-hydroxy-4-isopropoxy-benzamidine (75 mg, 0.328 mmol), 3-methyl-isonicotinic acid (41 mg, 0.298 mmol), HOBt (46 mg, 0.298 mmol), PS-carbodiimide (720 mg, 0.894 mmol, loading 1.24 mmol/g) was added ACN (3.5 mL) and diisopropylethylamine (156 μL, 0.894 mmol). The reaction vial was capped and heated at about 160° C. for about 30 min in a Biotage microwave. The reaction mixture was filtered and the resin was washed with ACN (4 mL). The filtrate was concentrated to dryness. The crude product was purified via reverse phase HPLC (30-90% ACN, 30 minute ramp) to give 10.2 mg (10%) of 4-[3-(3-chloro-4-isopropoxy-phenyl)-[1,2,4]oxadiazol-5-yl]-3-methyl-pyridine.

LC/MS (Table 1, Method c) Rₜ=2.70 min, m/z 330 (M+H)⁺; ¹H NMR (400 MHz, DMSO-d₆) δ 8.77 (s, 1H), 8.69 (d, 1H), 8.06 (d, 1H), 8.01 (dd, 2H), 7.39 (d, 1H), 4.83 (sept., 1H), 2.70 (s, 3H), 1.35 (d, 6H)

### General Procedure E

### Oxadiazole Formation from an Acid Chloride

To a solution of a 3-chloro-N-hydroxy-4-alkoxy-benzamidine (1-3 equivalents, preferably 1 equivalent) in pyridine is added a solution of an acid chloride (1-3 equivalents, preferably 2 equivalent) in pyridine. The reaction mixture is heated at 60-100° C. (preferably 100° C.) for a period of 8-24 h (preferably 20 h). The solvent is removed under reduced pressure and the residue is further purified by chromatography.

### Exemplification of General Procedure E

### Preparation of 3-[3-chloro-4-(1-ethyl-propoxy)-phenyl]-5-o-tolyl-[1,2,4]oxadiazole

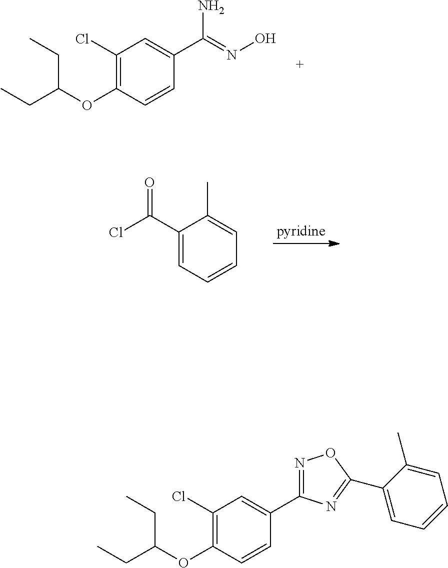

To a solution of 3-chloro-4-(1-ethyl-propoxy)-N-hydroxy-benzamidine (64 mg, 0.25 mmol) (prepared by General procedure B) in pyridine (1 mL) was added a solution of 2-methylbenzoyl chloride (77 mg, 0.5 mmol) in pyridine (1 mL). The mixture was heated at about 100° C. overnight. The solvent was removed under reduced pressure and the crude product was purified via SFC(CO₂/pure MeOH; gradient: 5% hold for 0.5 min, ramp at 7.3% to 50% over 6.5 min, hold at 50% for 1 min) to give 3-[3-chloro-4-(1-ethyl-propoxy)-phenyl]-5-o-tolyl-[1,2,4]oxadiazole (16.5 mg, 18.5%).

LC/MS (Table 1, Method b) Rₜ=3.18 min, m/z 356.13 (M−H)⁻; ¹H NMR (400 MHz, CHCl₃) δ 8.19 (d, 1H), 8.16 (dd, 1H), 8.01 (dd, 1H), 7.48 (m, 1H), 7.37 (d, 1H), 7.01 (d, 1H), 4.28 (m, 1H), 1.77 (m, 4H), 1.01 (t, 6H)

### Preparation of 3-(3-chloro-4-isopropoxyphenyl)-5-(3-chloropyridin-4-yl)-[1,2,4]-oxadiazole

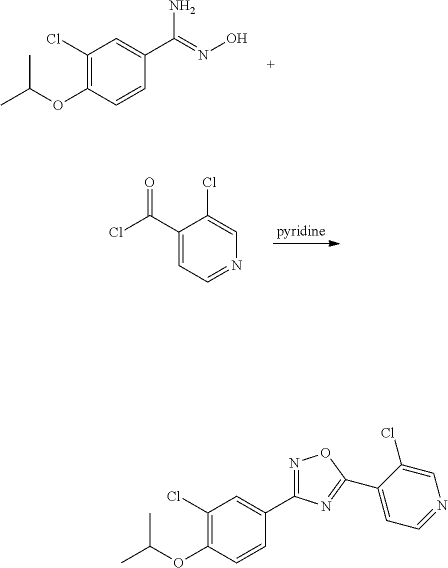

To a solution of 3-chloroisonicotinoyl chloride (about 2.6 mmol) (prepared by General procedure F) in pyridine (5 mL) was added 3-chloro-4-isopropoxy-N-hydroxy-benzamidine (300 mg, 1.31 mmol) (prepared by General procedure B). The mixture was heated at about 100° C. overnight. The solvent was removed under reduced pressure and the crude product was purified via normal phase silica gel chromatography (0-50% ethyl acetate/heptane gradient over 30 min) to give 3-(3-chloro-4-isopropoxyphenyl)-5-(3-chloropyridin-4-yl)-[1,2,4]-oxadiazole (323 mg, 70.3%).

LC/MS (Table 1, Method b) Rₜ=3.88 min, m/z 349.04 (M−H)⁻; ¹H NMR (400 MHz, CHCl₃) δ=8.84 (d, 1H), 8.69 (d, 1H), 8.11 (d, 1H), 8.02 (d, 1H), 7.99 (dd, 1H), 7.02, (d, 1H), 4.69 (m, 1H), 1.44 (d, 6H)

### General Procedure F

### Formation of an Acid Chloride from a Carboxylic Acid

To an acid (preferably 1 equivalent) in a suitable solvent, such as DCM or dichloroethane (preferably DCM) is added a chlorinating reagent such as thionyl chloride or oxalyl chloride (preferably thionyl chloride) (1-100 equivalents, preferably 3 equivalents). The reaction mixture is stirred at 20-80° C. (preferably at about 23° C.) for a period of 1-24 hours (preferably 3 hours). The solvent is removed under reduced pressure. The crude product is dried under vacuum and then subjected to General Procedure E.

### Exemplification of General Procedure F

### Preparation of 3-methyl-isonicotinoyl chloride

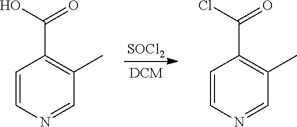

To 3-methylisonicotinic acid (100 mg, 0.729 mmol) suspended in DCM (2.5 mL) was added thionyl chloride (260 mg, 2.188 mmol). The reaction mixture was stirred at RT for about 3 h. The solvent was removed under reduced pressure and the residue was dried under high vacuum for 1 h to give 3-methyl-isonicotinoyl chloride.

### Preparation of 3-chloro-isonicotinoyl chloride

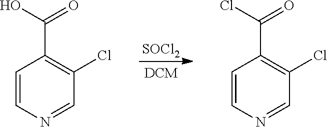

To 3-chloroisonicotinic acid (413 mg, 2.62 mmol) was added thionyl chloride (5 mL, 68.5 mmol). The reaction mixture was stirred at RT for about 20 h. The solution was concentrated under reduced pressure and the residue was dried under high vacuum for 1 h to give 3-chloro-isonicotinoyl chloride.

### General Procedure G

### Formation of Aldehyde from Nitrile

A mixture of a nitrile in a round bottom flask containing (0.9-1.2 equivalents, preferably 1.0 equivalents) in a suitable solvent such as dichloromethane or dichloroethane (preferably dichloromethane) was cooled to between 0° C. and −60° C. (preferably −40° C.). A solution of Dibal-H (0.9-2.5 equivalents, preferably 2.0 equivalents) was added dropwise and then the solution was stirred for 15-240 minutes (preferably 120 minutes), quenched with methanol, warmed to ambient temperature and treated with a 10% solution of Rochelle's salt. After extraction with DCM the combined organic layers were stirred with dilute aqueous acid (preferably 1M aqueous HCl). The layers were separated and the aqueous layer extracted with DCM. The combined organic layers were washed with brine, dried over MgSO₄ or Na₂SO₄, filtered and evaporated to dryness. The crude product is further purified by chromatography.

### Exemplification of General Procedure G

### Preparation of 3-chloro-4-(3-(3-chloro-4-isopropoxyphenyl)-1,2,4-oxadiazol-5-yl)benzonitrile

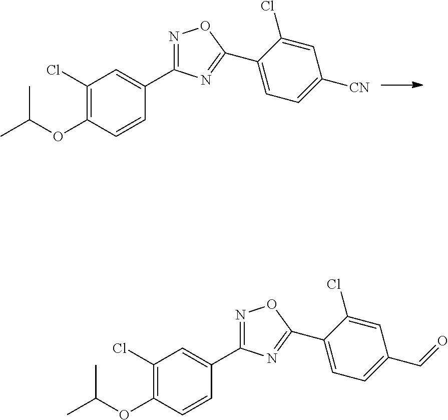

To a 100 mL round bottom flask equipped with septa cap outfitted with nitrogen inlet needle was charged with 3-chloro-4-(3-(3-chloro-4-isopropoxyphenyl)-1,2,4-oxadiazol-5-yl)benzonitrile (1.529 g, 3.27 mmol) in DCM (65.4 mL) to give an orange solution. The reaction mixture was cooled to about −40° C. via an ACN-dry ice bath. Dibal-H (3.60 mL, 3.60 mmol) was then added dropwise at about −40° C. The resulting mixture was stirred for about 2 h at about −40° C. Methanol (0.5 mL, 12.36 mmol) was then added dropwise to the reaction mixture at about −40° C. The ice bath was removed and the reaction was left to warm to ambient temperature then Rochelle's salt solution (60 mL) was added. The resulting mixture was stirred vigorously for about 3 h. The aqueous layer was separated. The organic phase was washed with brine, dried (MgSO₄) and concentrated to give crude yellow oil. The residue was purified via Analogix® FCC system using RediSep™ RS 120 g column, with a gradient of 0-15% EtOAc/Heptane over 40 min. at 40 mL/min then held at 15% until all peaks eluted. Fractions containing the product were combined and concentrated to yield 3-chloro-4-(3-(3-chloro-4-isopropoxyphenyl)-1,2,4-oxadiazol-5-yl)benzonitrile (0.791 g, 2.09 mmol). ¹H NMR (400 MHz, CDCl₃) δ ppm 10.09 (s, 1H), 8.35 (d, J=8.02 Hz, 1H), 8.21 (d, J=1.90 Hz, 1H), 8.09 (s, 1H), 8.03 (dd, J=8.56, 1.86 Hz, 1H), 7.94 (dd, J=8.04, 0.79 Hz, 1H), 7.05 (d, J=8.62 Hz, 1H), 4.69 (td, J=12.05, 6.04 Hz, 1H), 1.45 (t, J=6.80 Hz, 6H).

### General Procedure H

### Amination of Aldehyde

A mixture of an amine (0.9-1.2 equivalents, preferably 1.1 equivalents), an aldehyde (0.9-1.2 equivalents, preferably 1.0 equivalents), a suitable reducing agent, such as polymer supported sodium cyanoborohydride or sodium cyanoborohydride (preferably polymer supported sodium cyanoborohydride)(1.5-3.0 equivalents, preferably 2.0 equivalents), acetic acid (2-24 drops, preferably 6 drops) and a suitable solvent such as DCM or methanol (preferably DCM) was stirred at ambient temperature for 4-72 h, preferably 17 h. The crude product is further purified by chromatography.

### Alternate General Procedure H

In a 2.5 microwave vial, a solution of aldehyde (25 mg, 1 eq) dissolved in 1:1 DCM:methanol (1.5 mL) is added, followed by the addition of amine (35 mg, 1.2 eq.) also dissolved in 1:1 DCM:methanol solution (1.0 mL), followed by resin-bound cyanoborohydride (3 eq.) and acetic acid (10 eq.). The microwave vial was capped and heated at 90° C. for 600 seconds per reaction in Biotage parallel microwave optimizer. The reaction was checked by LC/MS (Table 1, method m) and concentrated to dryness. The residue was dissolved in 1:1 DMSO/MeOH and purified by reverse phase HPLC (Table 1, method L).

### Exemplification of General Procedure H

### Preparation of 1-(3-chloro-4-(3-(3-chloro-4-isopropoxyphenyl)-1,2,4-oxadiazol-5-yl)benzyl)-3-methylazetidine-3-carboxylic acid

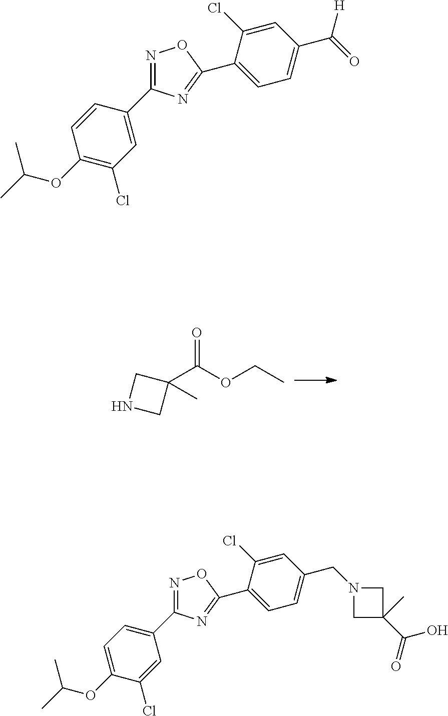

A 500 mL round bottom flask was charged with 3-chloro-4-(3-(3-chloro-4-isopropoxyphenyl)-1,2,4-oxadiazol-5-yl)benzaldehyde (0.745 g, 1.975 mmol), ethyl 3-methylazetidine-3-carboxylate (0.566 g, 3.95 mmol) (Tetrahedron Letters 1991, 32, 36, 4795-4798) and methanol (197 mL). To this was added acetic acid (0.904 mL, 15.80 mmol). The resulting mixture was stirred at ambient temperature for about 1 h then sodium cyanoborohydride (0.095 g, 1.512 mmol) was added in one portion. The reaction was stirred for about 17 h at ambient temperature. The progress of the reaction was monitored by LC/MS. The reaction was concentrated in vacuo to give crude dark yellow oil. The residue was purified via Analogix® FCC system using 120 g Redi-Sep column, with a gradient of 0-40% EtOAc/Heptane over about 45 min. at 50 mL/min then held at 40% EtOAc until all peaks eluted. Fractions containing product were combined and concentrated to yield 0.820 g (1.626 mmol) of colorless oil. The material was dissolved in THF (80 mL). To this was added NaOH (9.0 mL, 9.00 mmol) as 1N solution, followed by MeOH (about 25 mL). The reaction was stirred at ambient temperature for about 3 h, after which the LC/MS showed that hydrolysis was complete. To the reaction mixture was added HCl (9.0 mL, 9.00 mmol) as 1N solution dropwise to neutralize the pH. The reaction mixture was concentrated in vacuo then lyophilized to dryness. The crude white solid was triturated in diethyl ether and DCM then filtered. The resulting solid was washed with water then oven-dried overnight to give 1-(3-chloro-4-(3-(3-chloro-4-isopropoxyphenyl)-1,2,4-oxadiazol-5-yl)benzyl)-3-methylazetidine-3-carboxylic acid (0.377 g, 0.75 mmol) as white solid. LC/MS (Table 1, Method a) Rₜ=1.81 min.; MS m/z: 476.15 (M+H)⁺. ¹H NMR (400 MHz, DMSO) δ ppm 12.67-12.25 (m, 1H), 8.24-7.93 (m, 3H), 7.73-7.32 (m, 3H), 4.90-4.76 (m, 1H), 3.69 (s, 2H), 3.43 (d, J=6.51 Hz, 2H), 3.09 (d, J=6.43 Hz, 2H), 1.45 (s, 3H), 1.35 (d, J=5.75 Hz, 6H).

### General Procedure I

### Alkylation of Indole with Acrylate

To a solution of an indole (0.9-1.2 equivalents, preferably 1.0 equivalents) in a suitable solvent such as ACN at about 60° C. was added an acrylate (1.0-2.0 equivalents, preferably 1.5 equivalents) and a base such as DBU (0.3-1.0 equivalents, preferably 0.5 equivalents). The mixture was stirred at about 50° C. overnight. The solvent was removed under reduced pressure and the crude product was dissolved in DCM, washed with brine, dried over MgSO₄ or Na₂SO₄, filtered and the solvent was removed under reduced pressure. The crude product is further purified by chromatography or recrystallization.

### Exemplification of General Procedure I

### Preparation of tert-butyl 3-(4-(3-(3-chloro-4-isopropoxyphenyl)-1,2,4-oxadiazol-5-yl)-1H-indol-1-yl)propanoate

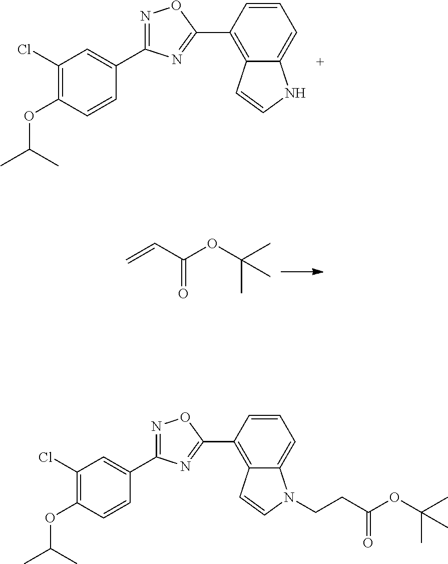

To a solution of 3-(3-chloro-4-isopropoxyphenyl)-5-(1H-indol-4-yl)-1,2,4-oxadiazole (5.6 g, 15.83 mmol) in ACN (55.9 mL) at about 60° C. was added tert-butyl acrylate (3.45 mL, 23.74 mmol) dropwise, followed by DBU (1.193 mL, 7.91 mmol). The mixture was stirred at about 50° C. overnight. The solvent was removed under reduced pressure and the crude product was dissolved in DCM (150 mL), washed with brine (3×100 mL) dried over MgSO₄, filtered and the solvent removed under reduced pressure. Re-crystallization from 30-60° C. petroleum ether gave tert-butyl 3-(4-(3-(3-chloro-4-isopropoxyphenyl)-1,2,4-oxadiazol-5-yl)-1H-indol-1-yl) propanoate (5.42 g, 69.6%). LC/MS (Table 1, Method b) Rₜ=3.03 min, m/z 482.26 (M+H)⁺.

### General Procedure J

### Alkylation of Indole with an Alkyl Halide

To a solution of an indole (0.9-1.2 equivalents, preferably 1.0 equivalents) in a suitable solvent such as DMF was added NaH (0.9-1.2 equivalents, preferably 1.1 equivalents). After about 15 min an alkyl halide (0.9-2.0 equivalents, preferably 1.5 equivalents) was added and the reaction mixture was heated to about 50° C. After about 24 h the reaction mixture was cooled to ambient temperature, evaporated to dryness and the crude product is further purified by chromatography.

### Exemplification of General Procedure J

### Preparation of tert-butyl 4-(4-(3-(3-chloro-4-isopropoxyphenyl)-1,2,4-oxadiazol-5-yl)-1H-indol-1-yl)butanoate

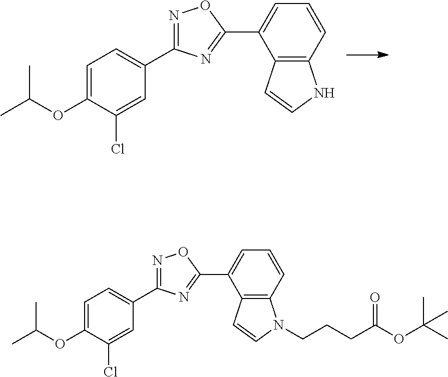

To a solution of 3-(3-chloro-4-isopropoxyphenyl)-5-(1H-indol-4-yl)-1,2,4-oxadiazole (0.100 g, 0.283 mmol) in DMF (0.999 mL) was added NaH (0.012 g, 0.311 mmol). After about 15 min tert-butyl 4-bromobutanoate (0.095 g, 0.424 mmol) was added and the reaction mixture was heated to about 50° C. After about 24 h the reaction mixture was cooled to ambient temperature, concentrated in vacuo and purified by chromatography on silica gel (eluting with EtOAc/Hep) to provide tert-butyl 4-(4-(3-(3-chloro-4-isopropoxyphenyl)-1,2,4-oxadiazol-5-yl)-1H-indol-1-yl)butanoate (0.135 g, 93%) as a colorless oil that solidified on standing. LC/MS (Table 1, Method c) Rₜ=3.50 min, m/z 496 (M+H)⁺.

### General Procedure K

### Deprotection of tert-butyl Ester or Ether

To a solution of a tert-butyl ester (0.9-1.2 equivalents, preferably 1.0 equivalents) in a suitable solvent such as DCM was added trifluoroacetic acid (15-25 equivalents, preferably 20 equivalents. The mixture was stirred at ambient temperature for about 1-10 h (preferably 8 h). The solvent was removed under reduced pressure and crude product was further purified by chromatography or recrystallization.

### Exemplification of General Procedure K

### Preparation of 3-(4-(3-(3-chloro-4-isopropoxyphenyl)-1,2,4-oxadiazol-5-yl)-1H-indol-1-yl)propanoic acid

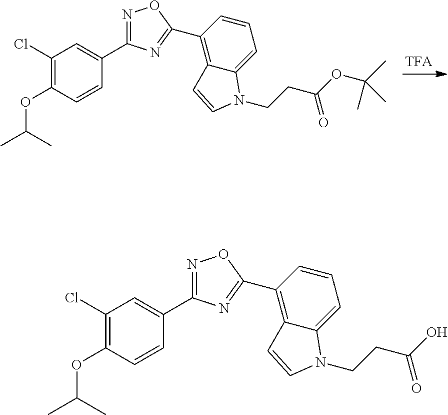

To a solution of tert-butyl 3-(4-(3-(3-chloro-4-isopropoxyphenyl)-1,2,4-oxadiazol-5-yl)-1H-indol-1-yl) propanoate oxadiazole (5.25 g, 10.89 mmol) in DCM (136 mL) was added trifluoroacetic acid (16.78 mL, 218 mmol). The mixture was stirred at ambient temperature for about 8 h. The solvent was removed under reduced pressure and the solid residue was triturated with ether to give 3-(4-(3-(3-chloro-4-isopropoxyphenyl)-1,2,4-oxadiazol-5-yl)-1H-indol-1-yl)propanoic acid (4.35 g, 93.0%). LC/MS (Table 1, Method b) Rₜ=3.03 min, m/z 356.13 (M−H)⁻; ¹H NMR (400 MHz, DMSO) δ 12.39 (s, 1H), 8.13 (m, 1H), 8.07 (m, 1H), 8.00 (d, 1H), 7.94 (d, 1H), 7.7 (d, 1H), 7.41 (m, 2H), 7.18 (d, 1H), 4.84 (s, 1H), 4.53 (td, 2H), 2.82 (td, 2H), 1.36 (d, 6H).

### General Procedure L

### Amination of Aryl Halide

To a reaction vessel is added an aryl fluoride or bromide (preferably fluoride) (0.9-1.2 equivalents, preferably 1.0 equivalents), an amine (0.9-1.5 equivalents, preferably 1.1 equivalents) potassium carbonate (0.9-3.0 equivalents, preferably 2.0 equivalents) and a suitable solvent such as DMF, DMA or DMSO (preferably DMF). The reaction vial is capped and heated either via microwave with cooling or in an oil bath (preferably via microwave) at 110-200° C. (preferably 160° C.) for 15 min to 4 days (preferably 30 min) The crude product is optionally isolated from the reaction mixture by dilution with water followed by filtration or neutralization to pH≦3 and extraction with a suitable organic solvent (ethyl acetate, dichloromethane, diethyl ether—preferably dichloromethane). The product can be further purified by chromatography or crystallization.

### Exemplification of General Procedure L

### Preparation of (1R,3S)-3-(4-(3-(3-chloro-4-isopropoxyphenyl)-1,2,4-oxadiazol-5-yl)phenylamino)cyclopentanecarboxylic acid

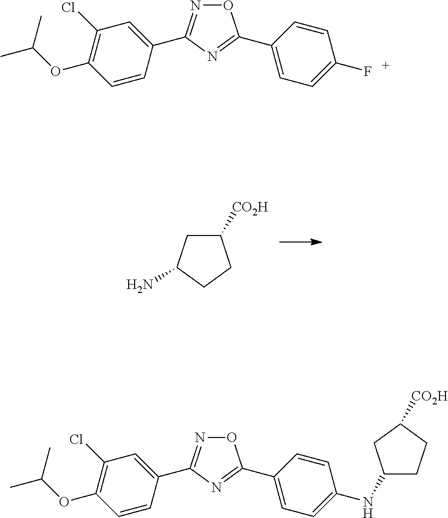

3-(3-Chloro-4-isopropoxyphenyl)-5-(4-fluorophenyl)-1,2,4-oxadiazole (360 mg, 1.082 mmol), (1R,3S)-3-aminocyclopentanecarboxylic acid (154 mg, 1.190 mmol), potassium carbonate (329 mg, 2.380 mmol) and DMF (2 mL) was heated with cooling at about 160° C. on the Biotage microwave for about 30 min. The mixture was diluted with DMSO (6 mL) and ACN (8 mL), filtered and divided into 8 aliquots for purification by molecular ion directed LC/MS. The fractions were combined and evaporated to afford a pale brown solid that was dried in vacuo at about 60° C. for about 3 h. This gave (1R,3S)-3-(4-(3-(3-chloro-4-isopropoxyphenyl)-1,2,4-oxadiazol-5-yl)phenylamino)cyclopentanecarboxylic acid (212 mg, 0.480 mmol, 44.3% yield) as a pale brown solid. LC/MS (Table 1, Method a) Rₜ=3.49 min, m/z 440.20 (M−H)−. 1H NMR (400 MHz, DMSO [symbol] ppm 4.81 (s, 1H), 3.96-3.76 (m, 1H), 2.78 (s, 1H), 2.42-2.25 (m, 1H), 2.12-1.95 (m, 1H), 1.89 (d, J=7.72 Hz, 2H), 1.73-1.61 (m, 1H), 1.61-1.48 (m, 1H), 1.39-1.30 (m, 7H), 12.22-12.07 (m, 1H), 6.73 (d, J=8.82 Hz, 2H), 6.87-6.79 (m, 1H), 7.36 (d, J=8.63 Hz, 1H), 7.87 (d, J=8.59 Hz, 2H), 7.98 (ddd, J=9.78, 1.97, 1.06 Hz, 2H).

### General Procedure M

### Alkylation of Phenol with an Alkyl Halide

A phenol such as benzyl 4-hydroxybenzoate (0.9-1.2 equivalents, preferably 1 equivalents) and a suitable base (such as potassium carbonate (1-5 equivalents, preferably 5 equivalents)) in an organic solvent (such as acetone (about 100 mL)) were combined. An alkyl bromide (such as tert-butyl 2-bromoacetate (0.9-1.2 equivalents, preferably 1 equivalents)) was added dropwise. The solution was stirred at about 60-70° C., preferably about 65° C. for 12-24 h, preferably about 18 h. The solution was cooled and the reaction mixture was filtered through a sintered glass funnel. The filtrate was concentrated in vacuo to afford a crude product which can be further purified via chromatography or crystallization.

### Exemplification of General Procedure M

### Preparation of benzyl 4-(2-tert-butoxy-2-oxoethoxy)benzoate

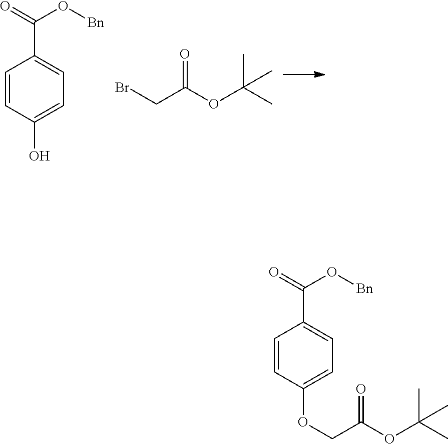

In a 100 mL round bottomed flask benzyl 4-hydroxybenzoate (1.445 g, 6.33 mmol) and potassium carbonate (4.17 g, 30.1 mmol) in acetone (100 mL) were combined. Tert-butyl 2-bromoacetate (0.908 mL, 6.03 mmol) was added dropwise. The solution was stirred at about 65° C. overnight. The solution was cooled and the reaction mixture was filtered through a sintered glass funnel. The filtrate was concentrated to afford pale yellow oil, which was purified via silica gel chromatography (40 g, 30% EtOAc:heptane) to afford benzyl 4-(2-tert-butoxy-2-oxoethoxy)benzoate (2.06 g, 5.90 mmol, 98% yield) as colorless oil. LC/MS (Table 1, Method a) Rₜ=4.31 min.

### General Procedure N

### Debenzylation

A high-pressure flask was charged with 5-20% (preferably 10%) palladium on carbon (0.9-1.2 equivalents, preferably 1.0 equivalents), a suitable solvent such as MeOH (200 mL), then a benzoate ester (50-70 equivalents, preferably 60 equivalents) were added. The resulting suspension was allowed to shake under an atmosphere of hydrogen about 10-70 psi (preferably 47 psi) at ambient temperature for about 2 h. The mixture was filtered through Celite® and the colorless filtrate was concentrated to afford the product.

### Exemplification of General Procedure N

### Preparation of 4-(2-tert-butoxy-2-oxoethoxy)benzoic acid

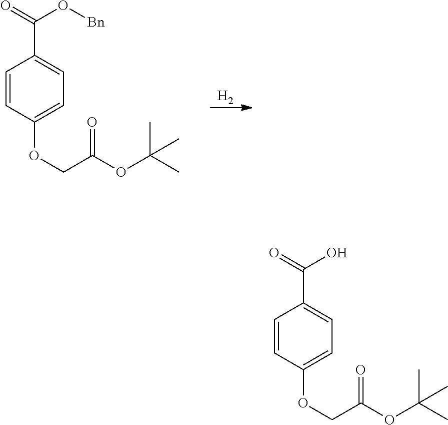

A 500 mL high-pressure flask was charged with benzyl 4-(2-tert-butoxy-2-oxoethoxy)benzoate (2.06 g, 6.02 mmol) in methanol (100 mL). 10% Palladium on carbon (0.320 g, 0.301 mmol) was added, the resulting suspension was allowed to shake under an atmosphere of hydrogen (about 47 psi) at RT for about 6 h. The mixture was filtered through Celite®, and the colorless filtrate was concentrated to afford 4-(2-tert-butoxy-2-oxoethoxy)benzoic acid (1.5 g, 5.95 mmol, 99% yield) as pale yellow solid. LC/MS (Table 1, Method a) Rₜ=3.03 min.; MS m/z: 251.30 (M−H)⁻. ¹H NMR (400 MHz, d-DMSO) ppm 7.88 (d, J=8.99 Hz, 2H), 6.98 (d, J=9.00 Hz, 2H), 4.75 (s, 2H), 1.43 (s, 9H).

### General Procedure O

### Deprotection of a Protected 1,2 diol

To a solution of protected diol (0.9-1.2 equivalents, preferably 1.0 equivalents) in a suitable solvent such as THF was added a solution of 1M HCl (1.5-2.5 equivalents, preferably 2.0 equivalents). The mixture was heated to about 70° C. for about 2 h. After cooling to ambient temperature a solution of an aqueous base such as 1M NaOH was added and the reaction mixture was concentrated in vacuo. The resulting solid was washed with water and dried in vacuo to afford the product.

### Exemplification of General Procedure O

### Preparation of 3-(4-(3-(3-chloro-4-isopropoxyphenyl)-1,2,4-oxadiazol-5-yl)phenoxy)propane-1,2-diol

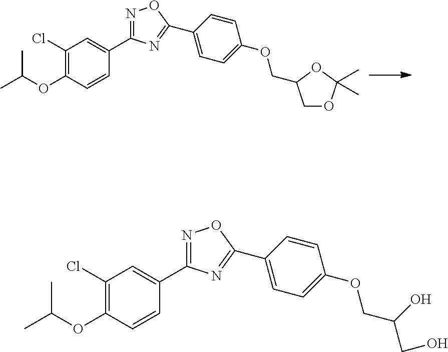

3-(3-Chloro-4-isopropoxyphenyl)-5-(4-((2,2-dimethyl-1,3-dioxolan-4-yl)methoxy)phenyl)-1,2,4-oxadiazole (0.1 g, 0.225 mmol) and p-toluenesulfonic acid monohydrate (8.55 mg, 0.045 mmol) were added in methanol (2.4 mL). The reaction mixture was heated at about 70° C. for about 16 h. The solution was cooled, methanol (1.5 mL) was added to the mixture and recrystallized, the resulted suspension was filtered, the solid was washed with water to afford 3-(4-(3-(3-chloro-4-isopropoxyphenyl)-1,2,4-oxadiazol-5-yl)phenoxy)propane-1,2-diol (0.08 g, 0.198 mmol, 88% yield) as white solid. LC/MS (Table 1, method g) Rₜ=2.97 min.; MS m/z: 405.18 (M+H)⁺. 1H NMR (400 MHz, DMSO-d6) δ ppm 8.16-8.09 (m, 2H), 8.05 (d, J=2.13 Hz, 1H), 7.99 (dd, J=8.64, 2.15 Hz, 1H), 7.38 (d, J=9.05 Hz, 1H), 7.25-7.16 (m, 2H), 5.03 (d, J=5.19 Hz, 1H), 4.87-4.78 (m, 1H), 4.72 (t, J=5.68 Hz, 1H), 4.15 (dd, J=3.97, 10.01 Hz, 1H), 4.01 (dd, J=6.20, 10.03 Hz, 1H), 3.84 (dt, J=4.04, 5.69, 5.91 Hz, 1H), 3.47 (t, J=5.84 Hz, 2H), 1.35 (d, J=6.03 Hz, 6H).

### General Procedure P

### Nucleophilic Displacement of an Aryl Fluoride

A solution of an aryl fluoride in a dry, aprotic solvent such as DMF, THF, toluene or 1,4-dioxane (preferably THF) is treated with a base such as sodium hydride (1.0 to 3.0 equivalents, preferably about 1.1 equivalents) at 0-50° C. (preferably RT). The mixture is treated dropwise with a solution of an alcohol (1-10 equivalents, preferably 1.0-1.1 equivalents) and then the reaction is stirred at 20-150° C. (preferably about 40° C.) until the aryl fluoride is consumed. The reaction is then cooled and concentrated then purified by extractive workup, crystallization or chromatography. Optionally, when one component of the coupling is protected, the crude material may be deprotected by hydrolysis during the extractive workup.

### Exemplification of General Procedure P

### Preparation of (R)-3-{3-Chloro-4-[3-(3-chloro-4-isopropoxy-phenyl)-[1,2,4]oxadiazol-5-yl]-phenoxy}-propane-1,2-diol

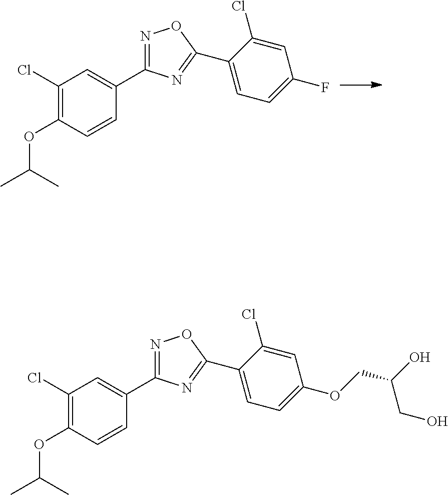

A solution of 5-(2-chloro-4-fluorophenyl)-3-(3-chloro-4-isopropoxyphenyl)-1,2,4-oxadiazole (6.25 g, 17.02 mmol) in dry THF (75.00 mL) is treated with NaH (1.225 g, 20.42 mmol) at room temperature. Neat (2,2-dimethyl-1,3-dioxolan-4-yl)methanol (2.330 mL, 18.72 mmol) is added dropwise at RT and the reaction is heated at about 40° C. for about 4 h. The reaction is cooled to RT and treated with 2 N HCl (75 mL) and allowed to stir 3 days at RT. The mixture was diluted with ethyl acetate (200 mL) and washed with saturated NaCl solution (3×100 mL), dried over sodium sulfate, filtered and concentrated. Further purification by crystallization from ethyl acetate and heptane yielded (R)-3-{3-Chloro-4-[3-(3-chloro-4-isopropoxy-phenyl)-[1,2,4]oxadiazol-5-yl]-phenoxy}-propane-1,2-diol (5.94 g, 79%) as a white solid. LC/MS (Table 1, Method A) Rₜ=2.60 min.; MS m/z: 498 (M+H)⁺. ¹H NMR (400 MHz, DMSO-d6) δ 8.15 (d, J=8.8, 1H), 8.05 (d, J=1.3, 1H), 8.08-7.95 (m, 1H), 7.39 (d, J=8.7, 1H), 7.33 (d, J=2.2, 1H), 7.19 (dd, J=8.9, 2.4, 1H), 5.05 (d, J=5.2, 1H), 4.87-4.78 (m, 1H), 4.72 (t, J=5.7, 1H), 4.20-4.02 (m, 2H), 3.83 (m, 1H), 3.46 (m, 2H), 1.35 (d, J=6.0, 6H).

### General Procedure Q

### Hydrolysis of an Ester

A solution of an ester in an organic solvent such as THF, MeOH, 1,4-dioxane, methanol, ethanol, DMF or DMSO (preferably THF, MeOH or 1,4-dioxane) is treated with an aqueous base (1-10 equivalents, preferably about 1 equivalent) such as NaOH, LiOH or KOH and the mixture is stirred at 20-100° C. (preferably RT) until the reaction is complete. The reaction is neutralized with an acid such as HCl, H₂SO₄, or acetic acid (preferably HCl), cooled, concentrated and purified by extractive workup, crystallization or chromatography.

### Exemplification of General Procedure Q

### Preparation of 3-{3-Chloro-4-[5-(5-chloro-6-isopropoxy-pyridin-3-yl)-[1,2,4]oxadiazol-3-yl]-phenoxy}-cyclobutanecarboxylic acid

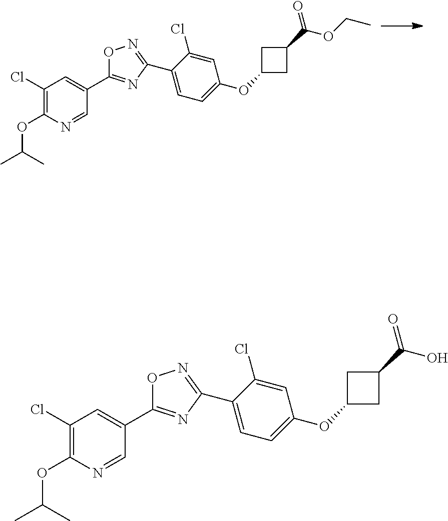

To a solution of (1r,3r)-ethyl 3-(3-chloro-4-(5-(5-chloro-6-isopropoxypyridin-3-yl)-1,2,4-oxadiazol-3-yl)phenoxy)cyclobutanecarboxylate (118 mg, 0.240 mmol) in 1,4-dioxane (2.00 mL) is added 2N NaOH (0.359 mL, 0.719 mmol) and the mixture is stirred at RT for about 3 hours. The reaction is neutralized by addition of 2N HCl (360 μL) and the mixture is concentrated. Re-crystallization from ACN and water yielded 3-{3-Chloro-4-[5-(5-chloro-6-isopropoxy-pyridin-3-yl)-[1,2,4]oxadiazol-3-yl]-phenoxy}-cyclobutanecarboxylic acid (72 mg, 65%) as a white solid.

LC/MS (Table 1, Method a) Rₜ=3.13 min.; MS m/z: 465/467/469 (M+H)⁺. ¹H NMR (400 MHz, DMSO-d6) δ 12.38 (bs, 1H), 8.91 (d, J=2.2, 1H), 8.53 (d, J=2.2, 1H), 7.99-7.92 (m, 1H), 7.15-7.10 (m, 1H), 7.08-7.01 (m, 1H), 5.48-5.42 (m, 2H), 5.01-4.94 (m, 2H), 3.17-3.05 (m, 1H), 2.74-2.66 (m, 3H), 2.46-2.28 (m, 2H), 1.42-1.32 (d, 6H).

### Exemplification of General Procedure Q

### Preparation of Deuterated methyl 5-chloro-6-isopropoxynicotinate

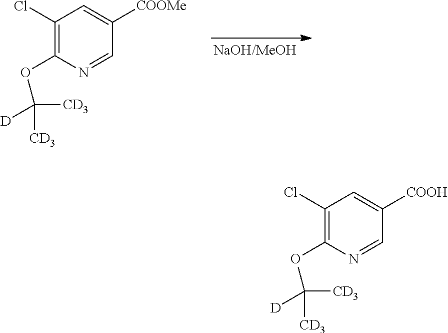

Methyl 5-chloro-6-hydroxynicotinate (3.86 g) was dissolved in about 21 mL methanol and about 2.1 mL water was added. 1.74 g sodium hydroxide was added to the solution. The mixture was heated to about 50° C. and mixed well to dissolve all NaOH and the solution was stirred for about 1 h at the same temperature. The aqueous methanol solution was extracted with hexane (2×10 mL) and the aqueous layer was adjusted to pH 4 using 6N HCl. Methanol was then removed by concentration and the aqueous slurry was adjusted to pH˜1 using HCl in an ice bath. The resulting slurry was stirred for about 30 min then filtered and washed with water. The product was further purified by drying under vacuum. Yield 3.64 g. HPLC purity 98%, 9.404 min. HPLC (Table 1, Method q)

### General Procedure R

### Mitsunobu Coupling to a Phenol

A solution of a phenol and alcohol (1-3 equivalents, preferably about 1.1 equivalents) in a solvent such as THF or 1,4-dioxane (preferably THF) is treated with a phosphine (1-5 equivalents) such as triphenylphosphine or resin-bound triphenylphosphine (preferably resin-bound triphenylphosphine) and optionally with 4 Å molecular sieves and the mixture is cooled to about 0° C. A solution of an alkylazodicarboxylate (1-2 equivalents, preferably about 1.1 equivalents) such as diethylazodicarboxylate, diisopropylazodicarboxylate, or di-tert-butylazodicarboxylate (preferably di-tert-butylazodicarboxylate) in a solvent such as THF or 1,4-dioxane (preferably THF) is added dropwise and then the reaction is warmed to 20-70° C. (preferably RT) with stirring for 1-24 h (preferably about 18 h). The reaction is filtered, concentrated and purified by chromatography on silica gel or crystallization.

### Preparation of 3-(3-Chloro-4-cyano-phenoxy)-cyclobutanecarboxylic acid tert-butyl ester

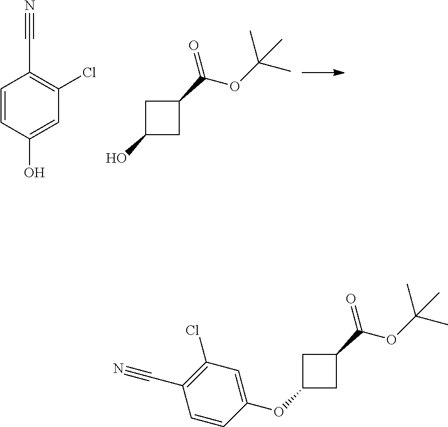

A solution of 2-chloro-4-hydroxybenzonitrile (5.00 g, 32.6 mmol) and (1s,3s)-tert-butyl 3-hydroxycyclobutanecarboxylate (6.17 g, 35.8 mmol) in THF (220 mL) is treated with 4-Å molecular sieves (10 g) and Ph₃P-resin bound (32.6 mL, 98 mmol) for about 20 minutes at RT, then cooled to about 0° C. A solution of di-tert-butyl azodicarboxylate (8.25 g, 35.8 mmol) in THF (30 mL) is added dropwise while maintaining the reaction temperature below about 4° C.

The reaction was stirred about 15 min at about 0° C. and then allowed to warm to RT for about 18 h. The reaction was filtered and the solids rinsed with methanol (3×50 mL) and the combined organic solutions were concentrated. The residue was purified on a silica gel column using a gradient from 10-30% ethyl acetate in heptane. The product fractions were combined and concentrated to a clear, colorless oil which solidifies on standing to afford 3-(3-chloro-4-cyano-phenoxy)-cyclobutanecarboxylic acid tert-butyl ester. LC/MS (Method a) Rₜ=2.75 min.; ¹H NMR (400 MHz, DMSO-d6) δ 7.87 (d, J=8.7, 1H), 7.19 (d, J=2.4, 1H), 6.99 (dd, J=8.7, 2.4, 1H), 4.99-4.92 (m, 1H), 3.11-3.04 (m, 1H), 2.69-2.63 (m, 2H), 2.40-2.28 (m, 2H), 1.44 (s, 9H).

### General Procedure S

### Deprotection of Phosphonate Ester with or without Bromination

To a solution of phosphonate ester (0.9-1.2 equivalents, preferably 1.0 equivalents) in an anhydrous organic solvent such as DCM, or THF (preferably DCM) is added bromotrimethylsilane (1-20 equiv, preferably 10 equiv). The reaction mixture is stirred for about 2-72 h (preferably 2-16 h). The reaction mixture is concentrated under reduced pressure. Intermediates and final compounds prepared via this General Procedure can be optionally purified using one or more of the Purification Methods described above.

### Exemplification of General Procedure S

### Example S.1 3-(4-(3-(3-chloro-4-isopropoxyphenyl)-1,2,4-oxadiazol-5-yl)phenylamino)propylphosphonic acid

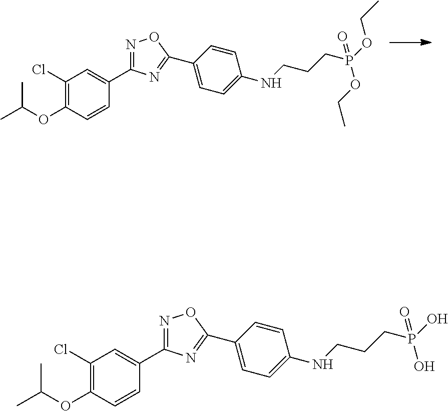

To a solution of diethyl 3-(4-(3-(3-chloro-4-isopropoxyphenyl)-1,2,4-oxadiazol-5-yl)phenylamino)propylphosphonate (Table D, #50, 0.060 g, 0.12 mmol) in DCM (1.2 mL) was added bromotrimethylsilane (0.15 mL, 1.18 mmol). The reaction mixture was stirred at RT for about 16 h. The reaction mixture was concentrated under reduced pressure. The material was purified by RP-HPLC (A=50 mM ammonium acetate, B=ACN; 5-100% B over about 30.0 min (21.0 mL/min flow rate); 21.2×250 mm Thermo Hyperprep C18 column, 8 μm particles) to give 3-(4-(3-(3-chloro-4-isopropoxyphenyl)-1,2,4-oxadiazol-5-yl)phenylamino)propylphosphonic acid (0.035 g, 65%): LC/MS (Table 1, Method b) Rₜ=1.83 min; MS m/z 452 (M+H)⁺.

### General Procedure T

### Preparation of Benzylic Olefin from Ketone

A suspension of 1-3 equivalents sodium hydride (60% in mineral oil, preferably 1.6 equivalent) in about 5 mL DMSO was heated at about 80-100° C. (preferably about 80° C.) for about 15-30 min (preferably 15 min) The mixture was cooled down to about 0-5° C. in an ice-bath, (4-(3-(3-chloro-4-isopropoxyphenyl)-1,2,4-oxadiazol-5-yl)benzyl)triphenylphosphonium chloride (preferably 1 equivalent) was added in one portion, the reaction mixture was stirred at RT for 30 min. A solution of ketone (preferably 1.2 equivalents) in DMSO was added dropwise. The reaction mixture was heated at about 80° C. for about 12-120 h (preferably 64 h). After cooling down, the mixture was poured into ice water, acidified by HCl (6N), extracted by DCM or ethyl acetate, the organic layer was washed by HCl (1N) and brine, dried over magnesium sulfate, filtration and concentration afford a crude product, which is further purified by chromatography.

### Exemplification of General Procedure T

### Preparation of ethyl 3-(4-(3-(3-chloro-4-isopropoxyphenyl)-1,2,4-oxadiazol-5-yl)benzylidene)cyclobutanecarboxylate

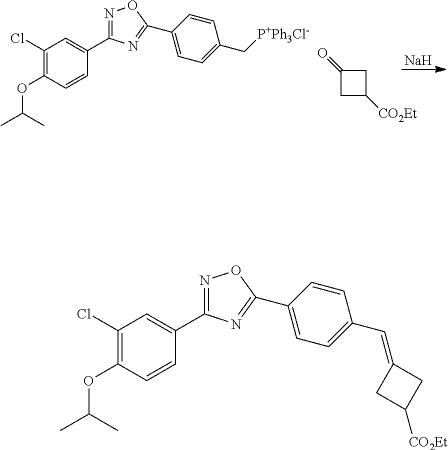

Sodium hydride (0.036 g, 0.900 mmol) was added in DMSO (5.6 mL) under nitrogen to give a white suspension. The mixture was heated at about 80° C. for about 18 min. It was cooled down to RT, then the reaction mixture was cooled down to about 0-5° C. by an ice-bath. (4-(3-(3-chloro-4-isopropoxyphenyl)-1,2,4-oxadiazol-5-yl)benzyl)triphenylphosphonium chloride (0.422 g, 0.675 mmol) was added in one portion, the ice-bath was removed and DMSO (2 mL) was added. It was stirred at RT for about 30 min, a solution of ethyl 3-oxocyclobutanecarboxylate (0.08 g, 0.563 mmol) in DMSO (1.0 mL) was added dropwise over about 5 min. The reaction mixture was heated at about 80° C. for about 64 h. The mixture was cooled down, poured into stirring ice-cold water (60 mL), HCl (6N, 20 mL) was added, the mixture was extracted by DCM (2×75 mL), the combined DCM layer was washed by HCl (1N, 40 mL), water (50 mL), brine (30 mL), dried over magnesium sulfate, filtered and concentrated afford 505 mg orange oil, which was purified via Analogix™ FCC (0% 5 min, 0-10% over 10 min, 10-15% over 15 min, 15% over 5 min EtOAc/Heptane; 80 g Redi-Sep® silica gel column) to afford ethyl 3-(4-(3-(3-chloro-4-isopropoxyphenyl)-1,2,4-oxadiazol-5-yl)benzylidene)cyclobutanecarboxylate (0.106 g, 0.234 mmol, 41.6% yield) as white solid. LC/MS (Table 1, Method g) Rₜ=2.07 min, m/z 453.12 (M+H)⁺. 1H NMR (400 MHz, DMSO-d6) δ ppm 8.10 (d, J=8.41 Hz, 2H), 8.04 (d, J=2.10 Hz, 1H), 7.98 (dd, J=8.63, 2.12 Hz, 1H), 7.46 (d, J=8.46 Hz, 2H), 7.37 (s, 1H), 6.31 (d, J=2.27 Hz, 1H), 4.81 (dt, J=6.0, 12.0 Hz, 1H), 4.11 (q, J=7.10 Hz, 2H), 3.41-3.22 (m, 3H), 3.17-3.03 (m, 2H), 1.34 (d, J=6.02 Hz, 6H), 1.20 (t, J=7.10 Hz, 3H).

### General Procedure U

### Reduction of Benzylic Olefin

A high-pressure flask was charged with a benzylic olefin (1 equivalent) and palladium on carbon (preferably 0.25 equivalents in ethyl acetate). The resulting suspension was stirred under an atmosphere of hydrogen (14 Psi) at ambient temperature for about 30-90 minutes (preferably 60 minutes). The mixture was filtered through Celite® and the colorless filtrate was concentrated to afford the product.

### Exemplification of General Procedure U

### Preparation of Ethyl 3-(4-(3-(3-chloro-4-isopropoxyphenyl)-1,2,4-oxadiazol-5-yl)benzyl)cyclobutanecarboxylate

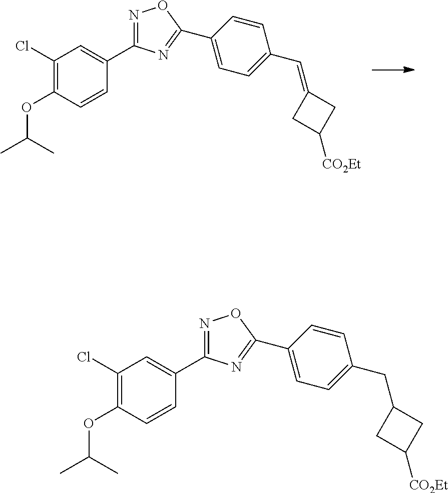

Ethyl 3-(4-(3-(3-chloro-4-isopropoxyphenyl)-1,2,4-oxadiazol-5-yl)benzylidene)cyclobutanecarboxylate (0.08 g, 0.177 mmol) and 10% palladium on carbon (0.047 g, 0.044 mmol) in EtOAc (8.83 mL) were added to give a black suspension. The reaction mixture was allowed to stir under an atmosphere of hydrogen balloon at RT for about 1 h. The reaction mixture was diluted with ethyl acetate and filtered, the filtrate was concentrated and the residue was used as is in next step. LC/MS (Table 1, Method i) Rₜ=2.08 min, m/z 455.14 (M+H)⁺.

### General Procedure V

### Hydrolysis of Ester

A solution of ester (1 equivalent) in ethyl alcohol/1,4-dioxane was combined with a solution of potassium/sodium hydroxide (1-10 equivalents, preferably 10 equivalent) in water, the mixture was heated at about 100° C. for about 16 h. The reaction mixture was cooled down, partitioned between aqueous ammonium chloride (1.0 M) and ethyl acetate, the organic layers were washed by water and concentrated in vacuo, the resulting in crude product which was further purified by chromatography.

### Exemplification of General Procedure V

### Preparation of 3-(4-(3-(3-chloro-4-isopropoxyphenyl)-1,2,4-oxadiazol-5-yl)benzyl)cyclobutanecarboxylic acid

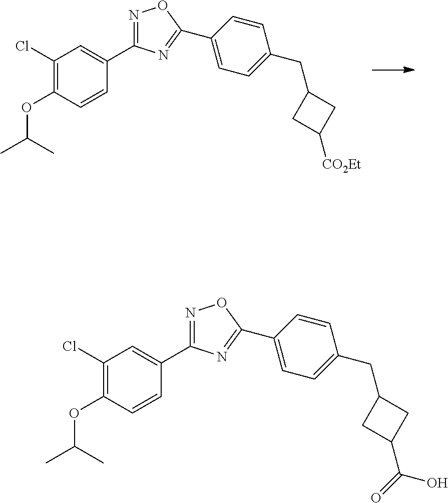

Ethyl 3-(4-(3-(3-chloro-4-isopropoxyphenyl)-1,2,4-oxadiazol-5-yl)benzyl)cyclobutanecarboxylate (0.08 g, 0.176 mmol) in EtOH (5 mL) was added to give a colorless solution. A solution of potassium hydroxide (0.099 g, 1.758 mmol) in water (2.5 mL) was added while stirring. The reaction mixture was heated at about 100° C. for about 16 h. The reaction mixture was cooled down, partitioned between aqueous ammonium chloride (1M, 40 mL) and EtOAc (2×35 mL), the combined organic layers were washed by water (2×30 mL), concentrated to afford 80 mg grey oil, which was purified by RP-HPLC (A=50 mM ammonium acetate, B=ACN; 30-100% B over 30.0 min (21.0 mL/min flow rate); 21.2×250 mm Thermo Hyperprep C18 column, 8 μm particles) to afford 3-(4-(3-(3-chloro-4-isopropoxyphenyl)-1,2,4-oxadiazol-5-yl)benzyl)cyclobutanecarboxylic acid (0.049 g, 0.112 mmol, 64% yield) as white solid. LC/MS (Table 1, Method g) Rₜ=3.13 min, m/z 427.24 (M+H)⁺. ¹H NMR (400 MHz, DMSO-d6) δ ppm 8.12-8.03 (m, 2H), 8.00 (dd, J=8.6, 2.1, 1H), 7.46 (t, J=8.4, 1H), 7.39 (d, J=8.9, 1H), 4.88-4.77 (m, 1H), 3.13-3.02 (m, 1H), 2.97-2.82 (m, 1H), 2.77 (d, J=7.5, 1H), 2.70-2.55 (m, 0H), 2.27-2.14 (m, 1H), 2.00-1.83 (m, 1H), 1.35 (d, J=6.0, 6H).

### General Procedure W

### Alkylation of a Phenol

A solution of ester (1 equivalent) in ethyl alcohol/1,4-dioxane or toluene is combined with a solution of potassium/sodium hydroxide or Ag₂CO₃ (1-10 equivalents, preferably 10 equivalent) in water, the mixture is heated at about 60-100° C. for about 16-20 hours. The reaction mixture is cooled down, and the resulting crude product mixture can be further purified by extractive workup and/or chromatography.

### Exemplification of General Procedure W

### Preparation of methyl 5-chloro-6-isopropoxynicotinate

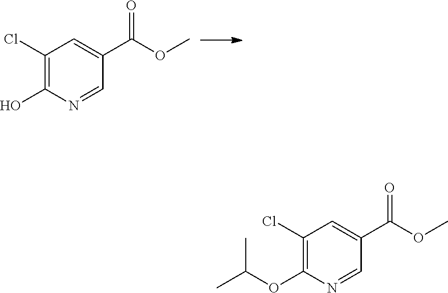

Methyl 5-chloro-6-hydroxynicotinate (8.91 g, 47.5 mmol) and 2-iodo-propane (7.12 mL, 71.2 mmol) were combined in toluene (202 mL) under nitrogen to give a colorless solution. Silver carbonate (19.65 g, 71.2 mmol) was added and the reaction heated at about 60° C. for about 4 h. TLC in 1:1 EtOAc/heptane showed (uv light visualization) that the reaction was not yet complete. The temperature was reduced to about 50° C. and the reaction stirred for about an additional 16 h. The reaction was allowed to cool to ambient temperature. The mixture was filtered through a buchner funnel and washed through with ethyl acetate. The solvents were removed under reduced pressure. The residue was purified by flash column chromatography (120 g Redi-Sep column) eluting with 5% ethyl acetate/heptane and the product fractions combined. The solvents were removed under reduced pressure to provide methyl 5-chloro-6-isopropoxynicotinate (10.539 g, 45.9 mmol, 97% yield) as a colorless oil that solidified under vacuum to a white solid: LC/MS (Method g) Rₜ=2.84 min.; MS m/z: 230.05 (M+H)⁺; ¹H NMR (400 MHz, CDCl3) δ ppm 8.71 (d, J=2.02 Hz, 1H), 8.22 (d, J=1.93 Hz, 1H), 5.53-5.42 (m, 1H), 3.94 (s, 3H), 1.44 (d, J=6.20 Hz, 6H).

### Exemplification of General Procedure W

### Preparation of methyl 5-chloro-6-deutero-isopropoxynicotinate

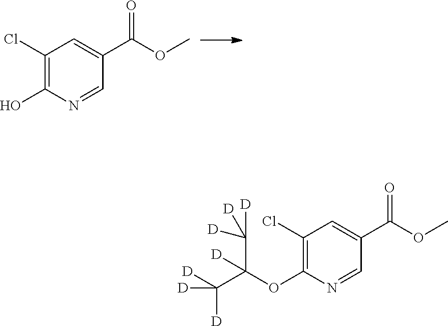

Methyl-5-chloro-6-hydroxynicotinate (3.15 g), toluene (70 mL) and deuterated isopropyl iodide (4.445 g) are added together at room temperature. Ag₂CO₃ (6.95 g) is then added and the reaction mixture is heated at about 60° C. for about 20 h. The solids are removed by filtration and washed with toluene (2×20 mL). The filtrate is concentrated to dryness and redissolved in with methanol followed by concentration each time (3×20 mL). HPLC purity 98%, 14.031 min. HPLC (Table 1, Method q)

### Tables Utilizing General Procedures

TABLE A
Examples made using General Procedures C, D, E (Scheme 2)
The letter in parentheses below the nitrile precursors indicates the General Procedure by which
the nitrile precursor was made.

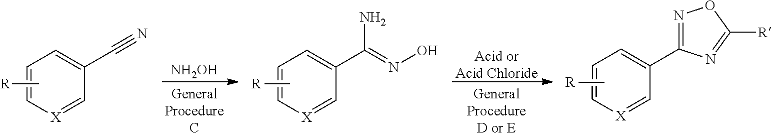

| | | Acid or | | | | |
|---|---|---|---|---|---|---|
| | Nitrile | acid | | | Rₜ/min | |
| Ex # | precursor | choride | Product | Name | (method) | m/z |
| A.1 | 3-chloro-4- isopropoxy benzonitrile (A) | 3-amino- 1-methyl- 1H- pyrazole- 5- carboxylic acid | 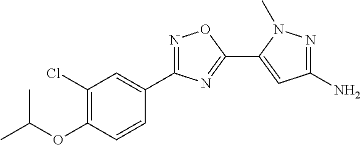 | 5-(3-(3- chloro-4- isopropoxy- phenyl)-1,2,4- oxadiazol-5- yl)-1-methyl- 1H-pyrazol-3- amine | 2.59 (g) | 334.10 (M + H)⁺ |
| A.2 | 3-chloro-4- isopropoxy- benzonitrile (A) | 1H- indole-5- carboxylic acid | 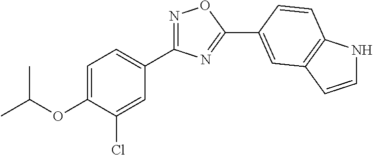 | 3-(3-chloro-4- isopropoxy- phenyl)-5-(1H- indol-5-yl)- 1,2,4- oxadiazole | 1.62 (c) | 354 (M + H)⁺ |
| A.3 | (E)-5- chloro-N′- hydroxy-6- isopropoxy nicotinimid- amide (A) | 2-methyl- 3- ((1S,4R)- 3-oxo-2- azabicyclo [2.2.1] heptan-2- yl)benzoic acid (Prep- aration #64, 65,66, 67) | 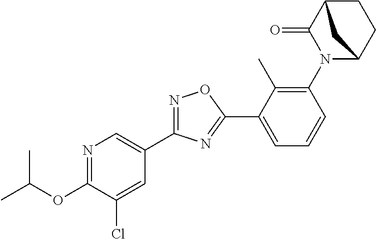 | (1S,4R)-2-(3- (3-(5-chloro-6- isopropoxy- pyridin-3-yl)- 1,2,4-oxa- diazol-5- yl)-2-methyl- phenyl)-2- azabicyclo [2.2.1]heptan- 3-one | 3.26 (a) | 439 (M + H)⁺ |
| A.4 | 3-chloro-4- isopropoxy- benzonitrile (A) | 3- cyano- benzoyl- chloride | 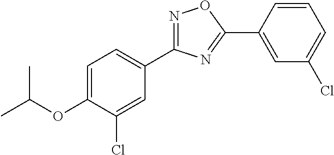 | 3-[3-(3-Chloro- 4-isopropoxy- phenyl)- [1,2,4]oxa- diazol-5-yl]- benzonitrile | 4.66 (g) | H¹NMR (DMSO- d) δ ppm 8.50 (s, 1 H), 8.41 (d, 1 H), 8.17 (s, 1 H), 7.98 (dd, 1 H), 7.88 (d, 1 H), 7.68 (t, 1 H), 7.03 (d, 1 H), 4.67 (m, 1 H), 1.42 (d, 6 H) |

TABLE B
Examples made using General Procedures C, D or E, G and H

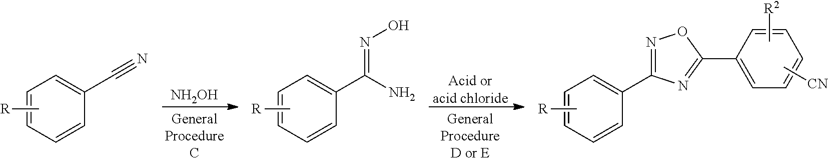

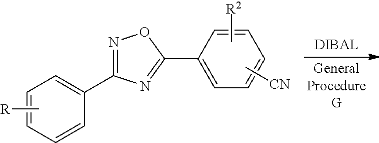

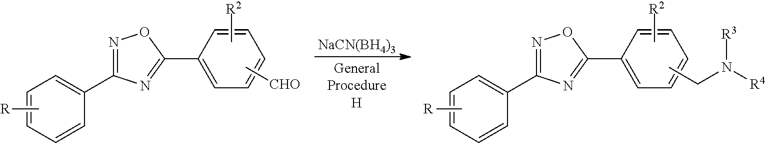

| | | Acid or | | | | | Rₜ/min |
|---|---|---|---|---|---|---|---|
| Ex | Nitrile | acid | | | | | (meth- |
| # | precursor | chloride | Amine | Structure | Name | m/z | od) |
| B.1 | 3-chloro- 4- isopro- poxy- benzo- nitrile | 4- cyano- benzoyl chloride | pyrrolidine- 3- carboxylic acid | 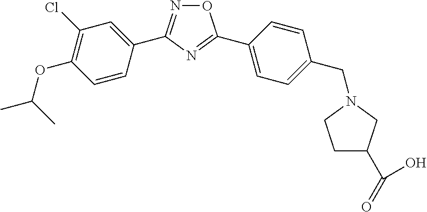 | 1-(4-(3-(3- chloro-4- isopropoxy- phenyl)-1,2,4- oxadiazol- 5-yl)benzyl) pyrrolidine- 3-carboxylic acid | 442.13 (M + H)+ | 1.97 (g) |
| B.2 | 3-chloro- 4- isopro- poxy- benzo- nitrile | 4- cyano- benzoyl- chloride | 3-(tert- butoxy carbonyl- amino) pyrrolidine 3- carboxylic acid | 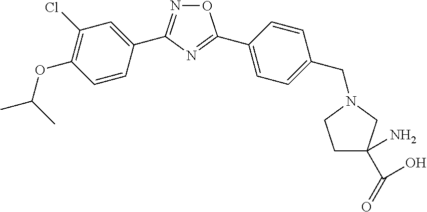 | 3-amino-1-(4- (3-(3-chloro- 4-isopropoxy- phenyl)-1,2,4- oxadiazol-5- yl)benzyl) pyrrolidine-3- carboxylic acid | 457.14 (M + H)+ | 1.94 (g) |
| B.3 | 4-(4- fluoro- piperidin- 1-yl)-3- (trifluoro- methyl) benzo- nitrile | 4- cyano- benzoyl- chloride | 1-(amino- methyl) cyclo- propane carboxylic acid | 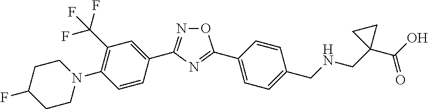 | 1-((4-(3-(4- (4-fluoro- piperidin-1- yl)-3-(tri- fluoromethyl) phenyl)-1,2,4- oxadiazol-5- yl)benzyl amino)methyl) cyclopropane- carboxylic acid | 519.17 (M + H)+ | 2.18 (g) |
| B.4 | 3-chloro- 4- isopro- poxy- benzo- nitrile | 4- cyano- benzoyl chloride | (S)- pyrrolidine- 3- carboxylic acid | 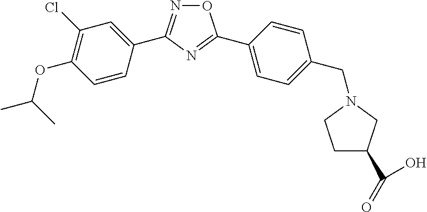 | (S)-1-(4- (3-(3-chloro- 4-isopropoxy- phenyl)-1,2,4- oxadiazol-5- yl)benzyl) pyrrolidine-3- carboxylic acid | 442.12 (M + H)+ | 1.97 (g) |
| B.5 | 3-chloro- 4- isopro- poxy- benzo- nitrile | 4- cyano- benzoyl chloride | (R)- pyrrolidine- 3- carboxylic acid | 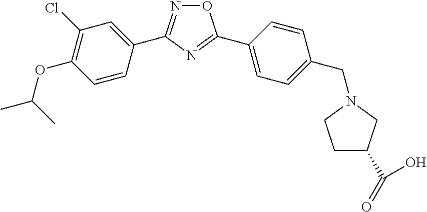 | (R)-1-(4-(3- (3-chloro-4- isopropoxy- phenyl)-1,2,4- oxadiazol-5- yl)benzyl) pyrrolidine-3- carboxylic acid | 442.08 (M + H)+ | 1.97 (g) |
| B.6 | 3-chloro- 4- isopro- poxy- benzo- nitrile | 4- cyano- benzoyl chloride | (S)- azetidine-2- carboxylic acid | 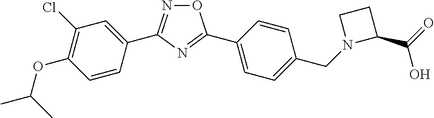 | (S)-1-(4-(3-(3- chloro-4- isopropoxy- phenyl)-1,2,4- oxadiazol-5- yl)benzyl) azetidine-2- carboxylic acid | 428.11 (M + H)+ | 1.96 (g) |
| B.7 | 4-(4- fluoro- piperidin- 1-yl)-3- (trifluoro- methyl) benzo- nitrile | 4- cyano- benzoyl chloride | (R)- pyrrolidine- 3- carboxylic acid | 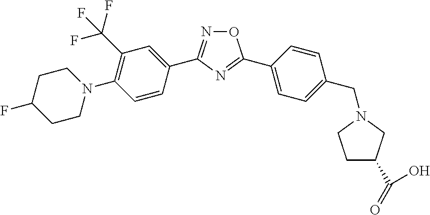 | (R)-1-(4-(3- (4-(4-fluoro- piperidin-1- yl)-3-(tri- fluoromethyl) phenyl)-1,2,4- oxadiazol-5- yl)benzyl) pyrrolidine-3- carboxylic acid | 519.10 (M + H)+ | 2.10 (g) |
| B.8 | 4-(4- fluoro piperidin- 1-yl)-3- (trifluoro- methyl) benzo- nitrile | 4- cyano- benzoyl chloride | (S)- pyrrolidine 3- carboxylic acid | 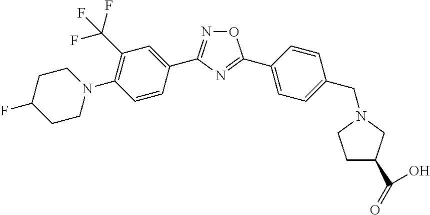 | (S)-1-(4-(3-(4- (4-fluoro- piperidin-1- yl)-3- (trifluoro- methyl) phenyl)-1,2,4- oxadiazole-5- yl)benzyl) pyrrolidine-3- carboxylic acid | 519.09 (M + H)+ | 2.10 (g) |
| B.9 | 4-(4- fluoro- piperidin- 1-yl)-3- (trifluoro- methyl) benzo- nitrile | 4- cyano- benzoyl chloride | 1- amino- cyclopro- pane- carboxylic acid | 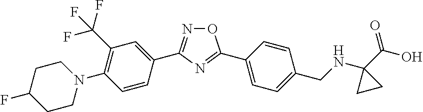 | 1-(4-(3-(4-(4- fluoro- piperidin-1- yl)-3- (trifluoro- methyl) phenyl)-1,2,4- oxadiazole-5- yl)benzyl amino)cyclo- propane- carboxylic acid | 505.08 (M + H)+ | 2.21 (g) |
| B.10 | 3-chloro- 4- isopro- poxy- benzo- nitrile | 4- cyano- benzoyl chloride | Cyclopropyl glycine | 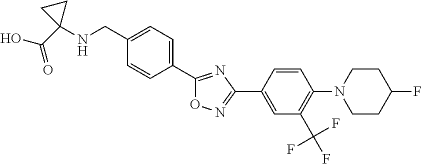 | 1-(4-{3-[4-(4- Fluoro- piperidin-1- yl)-3-trifluoro- methyl- phenyl]-[1,2, 4]oxadiazol- 5-yl}-benzyl amino)- cyclopropane carboxylic acid | 493.1 M + H | 1.66 (m) |
| B.11 | 3-chloro- 4- isopro- poxy- benzo- nitrile | 4- cyano- benzoyl chloride | Pyrrolidine 3- carboxylate | 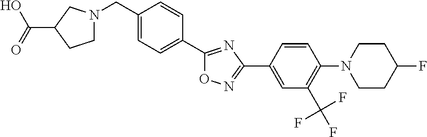 | 1-(4-{3-[4-(4- Fluoro- piperidin-1- yl)-3-tri- fluoromethyl- phenyl]-[1,2, 4]oxadiazol- 5-yl}-benzyl)- pyrrolidine-3- carboxylic acid | 519.1 M + H | 1.75 (m) |
| B.12 | 3-chloro- 4- isopro- poxy- benzo- nitrile | 4- cyano- benzoyl chloride | Pyrrolidine- 4- methyl-3- carboxylate | 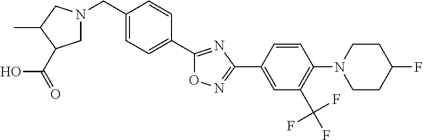 | 1-(4-{3-[4-(4- Fluoro- piperidin-1- yl)-3-tri- fluoromethyl- phenyl]-[1,2, 4]oxadiazol- 5-yl}-benzyl)- 4-methyl- pyrrolidine-3- carboxylic acid | 533.1 M + H | 1.73 (m) |
| B.13 | 3-chloro- 4- isopro- poxy- benzo- nitrile | 4- cyano- benzoyl chloride | Glycine | 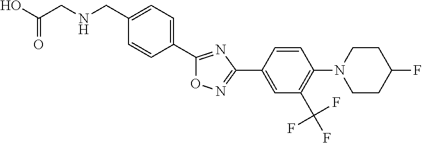 | (4-{3-[4-(4- Fluoro- piperidin-1- yl)-3-tri- fluoromethyl- phenyl]-[1,2, 4]oxadiazol- 5-yl}-benzyl)- amino)-acetic acid | 477.1 M − H | 1.71 (m) |
| B.14 | 3-chloro- 4- isopro- poxy- benzo- nitrile | 4- cyano- benzoyl chloride | (S)- pyrrolidine- 2-yl yl- acetic acid | 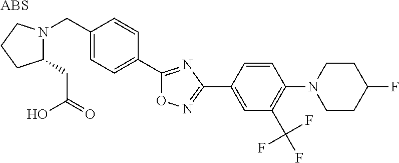 | [(S)-1-(4-{3- [4-(4-Fluoro- piperidin-1- yl)-3-trifluoro- methyl- phenyl]-[1,2, 4]oxadiazol- 5-yl}-benzyl)- pyrrolidin-2- yl]-acetic acid | 533.1 M + H | 1.72 (m) |
| B.15 | 3-chloro- 4- isopro- poxy- benzo- nitrile | 4- cyano- benzoyl chloride | (1- aminocyclo- propyl) methanol | 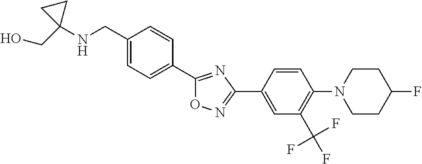 | [1-(4-{3-[4- (4-Fluoro- piperidin-1- yl)-3-tri- fluoromethyl- phenyl]-[1,2, 4]oxadiazol-5- yl}-benzyl amino)-cyclo propyl]- methanol | 491.1 M + H | 1.75 (m) |
| B.16 | 3-chloro- 4- isopro- poxy- benzo- nitrile | 4- cyano- benzoyl chloride | 4,4- dimethyl- pyrrolidine- 3- carboxylate | 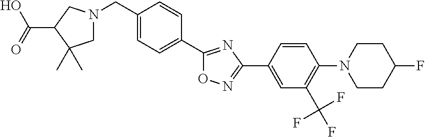 | 1-(4-{3-[4- (4-Fluoro- piperidin-1- yl)-3-tri fluoromethyl- phenyl]-[1,2, 4]oxadiazol-5- yl}-benzyl)- 4,4-dimethyl- pyrrolidin-3- carboxylic acid | 547.1 M + H | 1.78 (m) |
| B.18 | 3-chloro- 4- isopro- poxy- benzo- nitrile | 4- cyano- benzoyl chloride | 1-amino methyl cyclopro- panol | 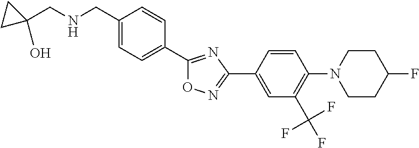 | 1-[(4-{3-[4- (4-Fluoro- piperidin-1- yl)-3-tri- fluoromethyl- phenyl]-[1,2, 4]oxadiazol-5- yl}-benzyl amino)- methyl]-cyclo- propanol | 491.1 M + H | 1.71 (m) |

TABLE C
Examples made using General Procedures C, D or E, I or J and K

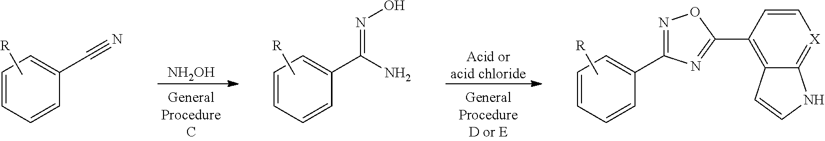

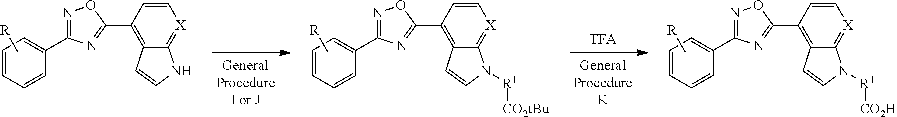

| | | Acid or | tert- | | | | Rₜ/min |
|---|---|---|---|---|---|---|---|
| Ex | Nitrile | acid | butyl | | | | (me- |
| # | precursor | chloride | ester | Structure | Name | m/z | thod) |
| C.1 | 3-chloro- 4-iso- propoxy- benzo- nitrile | 1H- indole-4- carboxylic acid | tert- butyl 4- bromo- butanoate | 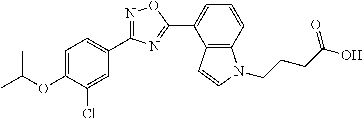 | 4-(4-(3- (3- chloro-4- isopropoxy- phenyl)- 1,2,4- oxadiazol-5- yl)-1H- indol-1- yl)butanoic acid | 440 (M + H)⁺ | 2.95 (a) |
| C.2 | 3-chloro- 4-iso- propoxy- benzo- nitrile | 1H- indole-4- carboxylic acid | tert- butyl 2- fluoro- acrylate | 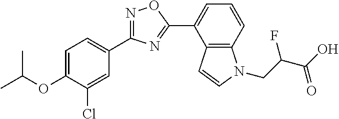 | 3-(4-(3- (3- chloro-4- isopropoxy- phenyl)- 1,2,4- oxadiazol- 5-yl)- 1H-indol- 1-yl)-2- fluoro- propanoic acid | 444 (M + H)⁺ | 2.19 (a) |
| C.3 | 3-chloro- 4-iso- propoxy- benzo- nitrile | 1H- indole-4- carboxylic acid | tert- butyl meth acrylate | 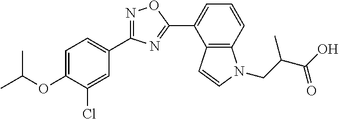 | 3-(4-(3- (3- chloro-4- isopropoxy phenyl)- 1,2,4- oxadiazol- 5-yl)- 1H-indol- 1-yl)-2- methyl- propanoic acid | 442 (M + H)⁺ | 2.47 (a) |
| C.4 | 3-chloro- 4-iso- propoxy- benzo- nitrile | 1H- indole-4- carboxylic acid | ethyl 3- chloro- 2,2- dimethyl- pro- panoate | 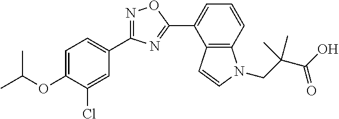 | 3-(4-(3- (3- chloro-4- isopropoxy- phenyl)- 1,2,4- oxadiazol- 5-yl)- 1H-indol- 1-yl)-2,2- dimethyl propanoic acid | 454 (M + H)⁺ | 3.11 (a) |
| C.5 | 3-chloro- 4-iso- propoxy- benzo- nitrile | 1H- indole-4- carboxylic acid | tert- butyl acrylate | 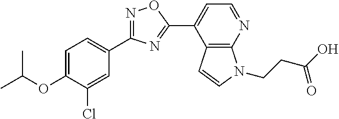 | 3-(4-(3- (3- chloro-4- iso- propoxy- phenyl)- 1,2,4- oxadiazol- 5-yl)- 1H- pyrrolo[2,3- b]pyridin-1- yl)propanoic acid | 427 (M + H)⁺ | 2.84 (a) |

TABLE D
Examples made using General Procedures C, D or E and L

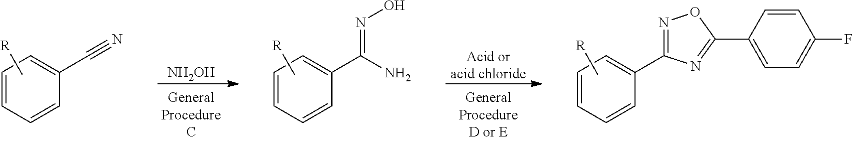

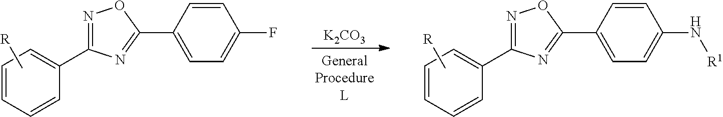

| | | | | | | | Rₜ/ |
|---|---|---|---|---|---|---|---|
| | Nitrile | | | | | | min |
| Ex | pre- | Acid | | | | | (me- |
| # | cursor | chloride | Amine | Structure | Name | m/z | thod) |
| D. 1 | 5- Chloro- 6-iso- pro- poxy- nico- tino- nitrile | 4- Fluoro- 2- methyl- benzoyl chloride | (1R,3S)- 3- amino- cyclo- pentane- carbo- xylic acid | 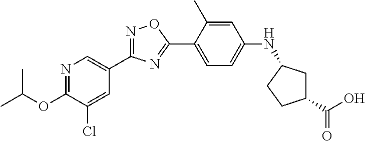 | (1R,3S)-3- {4-[3-(5- Chloro- 6- iso- propoxy- pyridin-3- yl)-[1,2,4] oxadiazol- 5-yl]-3- methyl- phenyl- amino}- cyclo- pentane- carbo- xylic acid | 458 (M − H)⁺ | 3.29 (a) |
| D. 2 | 3- chloro- 4-iso- pro- poxy- benzo- nitrile | 4- fluoro- benzoyl chloride | 4- amino- 3,3- di- methyl- butanoic acid | 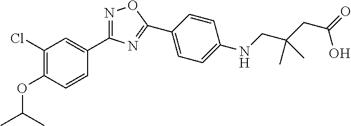 | 4-(4-(3-(3- chloro-4- isopropoxy phenyl)- 1,2,4- oxadiazol- 5-yl) phenyl- amino)- 3,3- dimethyl- butanoic acid | 444 (M − H)⁺ | 2.84 |
| D. 3 | 3- chloro- 4-iso- pro- poxy- benzo- nitrile | 4- fluoro- benzoyl chloride | 4- amino- buta- noic acid | 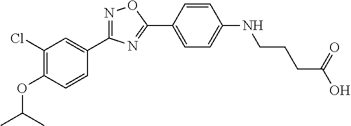 | 4-(4-(3-(3- chloro-4- isopropoxy phenyl)- 1,2,4- oxadiazol- 5-yl) phenyl- amino) butanoic acid | 416.17 (M + H)⁺ | 2.81 (g) |
| D. 4 | 3- chloro- 4-iso- pro- poxy- benzo- nitrile | 4- fluoro- benzoyl chloride | 2- (pyrro- lidin- 3- carbo- xylic acid | 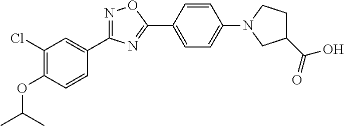 | 1-(4-(3-(3- chloro-4- isopropoxy phenyl)- 1,2,4- oxadiazol- 5-yl) phenyl) pyrro- lidine- 3- carboxylic acid | 428.7 (M + H)⁺ | 2.87 (g) |
| D. 5 | 3- chloro- 4-iso- pro- poxy- benzo- nitrile | 4- fluoro- benzoyl chloride | 2- (pyrro- lidin- 3- yl)acetic acid | 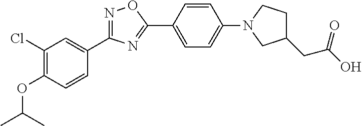 | 2-(1-(4-(3- (3-chloro- 4- isopropoxy phenyl)- 1,2,4- oxadiazol- 5-yl) phenyl) pyrrolidin- 3-yl) acetic acid | 442.18 (M + H)⁺ | 3.08 (g) |
| D. 6 | 3- bromo- 4-iso- pro- poxy- benzo- nitrile | 4- fluoro- benzoyl chloride | (1R,3S)- 3- amino- cyclo- pentane- carbo- xylic acid | 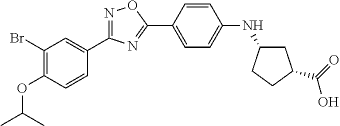 | (1R,3S)-3- (4-(3-(3- bromo-4- isopropoxy phenyl)- 1,2,4- oxadiazol- 5-yl) phenyl- amino) cyclo- pentane- carboxylic acid | 486.19 & 488.12 (M + H)⁺ | 3.01 (g) |
| D. 7 | 4- iso- pro- poxy- 3- (tri- fluoro- methyl) benzo- nitrile | 4- fluoro- benzoyl chloride | (1R,3S)- 3- amino- cyclo- pentane- carbo- xylic acid | 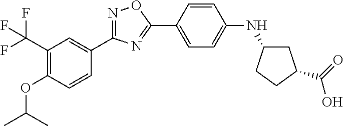 | (1R,3S)-3- (4-(3-(4- iso- propoxy- 3- (trifluoro- methyl) phenyl)- 1,2,4- oxadiazol- 5- yl)phenyl- amino) cyclo- pentane- carboxylic acid | 476.28 (M + H)⁺ | 2.98 (g) |
| D. 8 | 5- chloro- 6-iso- pro- poxy- nico- tino- nitrile | 4- fluoro- benzoyl chloride | (1R,3S)- 3- amino- cyclo- pentane- carbo- xylic acid | 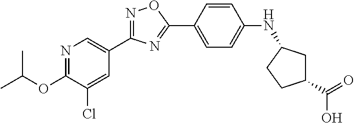 | (1R,3S)-3- (4-(3-(5- chloro-6- isopropoxy pyridin-3- yl)-1,2,4- oxadiazol- 5- yl)phenyl- amino) cyclo- pentane- carboxylic acid | 443 (M + H)⁺ | 1.58 (a) |
| D. 9 | 3- chloro- 4-iso- pro- poxy- benzo- nitrile | 4- fluoro- benzoyl chloride | (R)- pyrro- lidin- 3- amine | 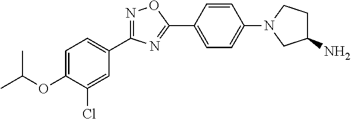 | (R)-1-(4- (3-(3- chloro-4- isopropoxy phenyl)- 1,2,4- oxadiazol- 5- yl)phenyl) pyrrolidin- 3-amine | 399 (M + H)⁺ | 1.98 (a) |
| D. 10 | 3- chloro- 4-iso- pro- poxy- benzo- nitrile | 4- fluoro- benzoyl chloride | (1R,2S)- 2- amino- cyclo- pentane- carbo- xylic acid, hydro- chloric acid | 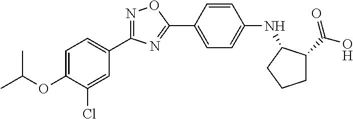 | (1R,2S)-2- (4-(3-(3- chloro-4- isopropoxy phenyl)- 1,2,4- oxadiazol- 5- yl)phenyl- amino) cyclo- pentane- carboxylic acid | 442 (M + H)⁺ | 2.82 (a) |
| D. 11 | 3- chloro- 4- isopro- poxy- benzo- nitrile | 4- fluoro- benzoyl chloride | (1S,2R)- 2- amino- cyclo- hexane- carbo- xylic acid, hydro- chloric acid | 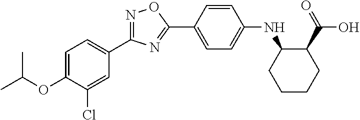 | (1S,2R)-2- (4-(3-(3- chloro-4- isopropoxy phenyl)- 1,2,4- oxadiazol- 5- yl)phenyl- amino) cyclo- hexane- carboxylic acid | 456 (M + H)⁺ | 2.94 (a) |
| D. 12 | 3- chloro- 4-iso- pro- poxy- benzo- nitrile | 4- fluoro- benzoyl chloride | (S)-1- (3- amino- pyrro- lidin- 1-yl) ethanone | 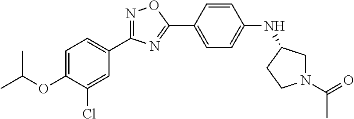 | (S)-1-(3-(4- (3-(3- chloro-4- isopropoxy phenyl)- 1,2,4- oxadiazol- 5- yl)phenyl- amino) pyrrolidin- 1- yl)ethanone | 441 (M + H)⁺ | 2.62 (a) |
| D. 13 | 3- chloro- 4-iso- pro- poxy- benzo- nitrile | 4- fluoro- benzoyl chloride | (1R,2R)- 2- amino- cyclo- hexane- carbo- xylic acid | 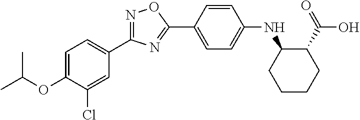 | (1R,2R)-2- (4-(3-(3- chloro-4- isopropoxy phenyl)- 1,2,4- oxadiazol- 5- yl)phenyl- amino) cyclo- hexane- carboxylic acid | 456 (M + H)⁺ | 2.88 (a) |
| D. 14 | 3- chloro- 4-iso- pro- poxy- benzo- nitrile | 4- fluoro- benzoyl chloride | (1R,2S)- 2- amino- cyclo- hexane- carbo- xylic acid | 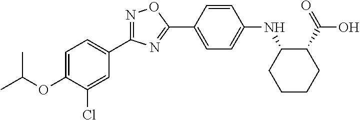 | (1R,2S)-2- (4-(3-(3- chloro-4- isopropoxy phenyl)- 1,2,4- oxadiazol- 5- yl)phenyl- amino) cyclo- hexane- carboxylic acid | 456 (M + H)⁺ | 2.94 (a) |
| D. 15 | 3- chloro- 4-iso- pro- poxy- benzo- nitrile | 4- fluoro- benzoyl chloride | 2- amino- cyclo- hexane- carbo- xylic acid | 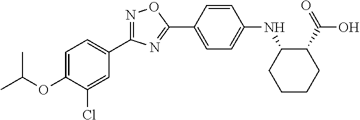 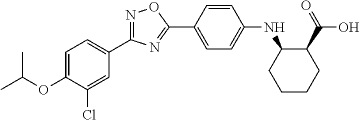 | (1R,2S)-2- (4-(3-(3- chloro-4- isopropoxy phenyl)- 1,2,4- oxadiazol- 5- yl)phenyl- amino) cyclo- hexane- carboxylic acid compound with (1S,2R)-2- (4-(3-(3- chloro-4- isopropoxy phenyl)- 1,2,4- oxadiazol- 5- yl)phenyl- amino) cyclo- hexane- carboxylic acid (1:1) | 456 (M + H)⁺ | 2.94 (a) |
| D. 16 | 3- chloro- 4-iso- pro- poxy- benzo- nitrile | 4- fluoro- benzoyl chloride | 2- amino- cyclo- hexane- carbo- xylic acid | 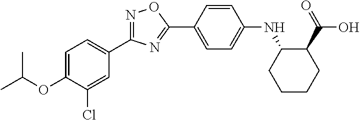 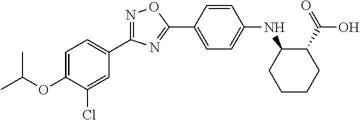 | (1R,2R)-2- (4-(3-(3- chloro-4- isopropoxy phenyl)- 1,2,4- oxadiazol- 5- yl)phenyl- amino) cyclo- hexane- carboxylic acid compound with (1S,2S)-2- (4-(3-(3- chloro-4- isopropoxy phenyl)- 1,2,4- oxadiazol- 5- yl)phenyl- amino) cyclo- hexane- carboxylic acid (1:1) | 456 (M + H)⁺ | 2.88 (a) |
| D. 17 | 3- chloro- 4-iso- pro- poxy- benzo- nitrile | 4- fluoro- benzoyl chloride | (1S,2R)- 2- amino- cyclo- pentane- carbo- xylic acid, Hydro- chloric Acid, 0.5H2O | 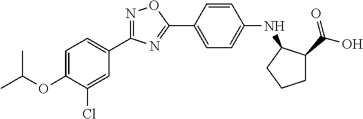 | (1S,2R)-2- (4-(3-(3- chloro-4- isopropoxy phenyl)- 1,2,4- oxadiazol- 5- yl)phenyl- amino) cyclo- pentane- carboxylic acid | 442 (M + H)⁺ | 2.83 (a) |
| D. 18 | 3- chloro- 4- iso- pro- poxy- benzo- nitrile | 4- fluoro- benzoyl chloride | (1S,2S)- 2- amino- cyclo- hexane- carbo- xylic acid | 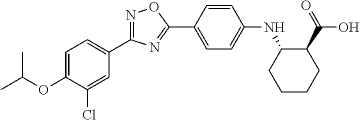 | (1S,2S)-2- (4-(3-(3- chloro-4- isopropoxy phenyl)- 1,2,4- oxadiazol- 5- yl)phenyl- amino) cyclo- hexane- carboxylic acid | 456 (M + H)⁺ | 2.88 (a) |
| D. 19 | 3- chloro- 4- iso- pro- poxy- benzo- nitrile | 4- fluoro- benzoyl chloride | (S)-1- (methyl- sulfonyl) pyrro- lidin- 3- amine | 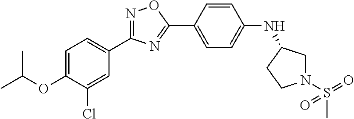 | (S)-N-(4- (3-(3- chloro-4- isopropoxy phenyl)- 1,2,4- oxadiazol- 5- yl) phenyl)- 1- (methyl- sulfonyl) pyrrolidin- 3- amine | 477 (M + H)⁺ | 2.78 (a) |
| D. 20 | 3- chloro- 4-iso- pro- poxy- benzo- nitrile | 4- fluoro- benzoyl chloride | (S)-2- (3- amino- pyrro- lidin- 1- yl)acetic acid, Tri- fluoro- acetic Acid | 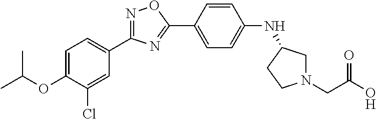 | (S)-2-(3-(4- (3-3- chloro-4- isopropoxy phenyl)- 1,2,4- oxadiazol- 5- yl)phenyl- amino) pyrro- lidin-1- yl)acetic acid | 457 (M + H)⁺ | 2.15 (a) |
| D. 21 | 3- chloro- 4-iso- pro- poxy- benzo- nitrile (Ge- neral Pro- cedure A) | 3- bromo- 4- fluoro- benzoic acid | (1R,3S)- 3- amino- cyclo- pentane- carbo- xylic acid | 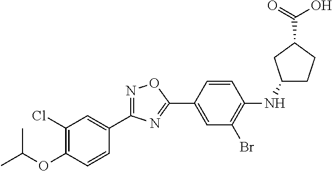 | (1R,3S)-3- (2-bromo- 4-(3-(3- chloro-4- isopropoxy phenyl)- 1,2,4- oxadiazol- 5- yl)phenyl- amino) cyclo- pentane- carboxylic acid | 522 (M + H)⁺ | 3.31 (g) |
| D. 22 | 3- choro- 4-iso- pro- poxy- benzo- nitrile (A) | 2- bromo- 3- fluoro- benzoic acid | (1R,3S)- 3- amino- cyclo- pentane- carbo- xylic acid | 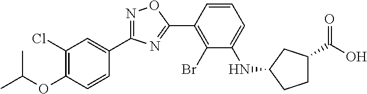 | (1R,3S)-3- (2-bromo- 3-(3-(3- chloro-4- isopropoxy phenyl)- 1,2,4- oxadiazol- 5- yl)phenyl- amino) cyclo pentane- carboxylic acid | 522 (M + H)⁺ | 3.17 (g) |
| D. 23 | 3- chloro- 4-iso- pro- poxy- benzo- nitrile | 6- chloro- nicotinic acid | (1R,3S)- 3- amino- cyclo- pentane- carbo- xylic acid | 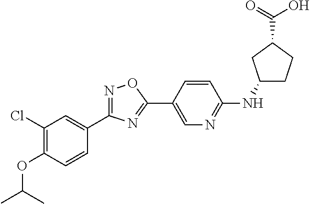 | (1R,3S)-3- (5-(3-(3- chloro-4- isopropoxy phenyl)- 1,2,4- oxadiazol- 5- yl)pyridin- 2- ylamino) cyclo- pentane carboxylic acid | 443.17 (M + H)⁺ | 2.61 (g) |
| D. 24 | 3- chloro- 4-iso- pro- poxy- benzo- nitrile | 2- fluoro- iso- nicotinic acid | (1R,3S)- 3- amino- cyclo- pentane- carbo- xylic acid | 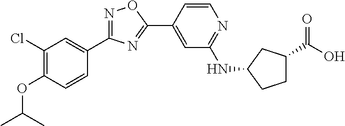 | (1R,3S)-3- (4-(3-(3- chloro-4- isopropoxy phenyl)- 1,2,4- oxadiazol- 5- yl)pyridin- 2- ylamino) cyclo- pentane carboxylic acid | 443.16 (M + H)⁺ | 2.73 (g) |
| D. 25 | 3- chloro- 4-iso- pro- poxy- benzo- nitrile | 4- fluoro- benzoyl chloride | (3S)- methyl 3- amino- 1- methyl cyclo- pentane- carbo- xylate | 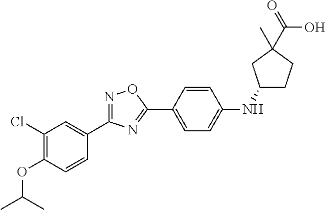 | (3S)-3-(4- (3-(3- chloro-4- isopropoxy phenyl)- 1,2,4- oxadiazol- 5- yl)phenyl- amino)-1- methyl- cyclo- pentane- carboxylic acid | 456.13 (M + H)⁺ | 3.02, 3.14 (g) |
| D. 26 | 3- chloro- 4-iso- pro- poxy- benzo- nitrile | 4- fluoro- benzoyl chloride | 2- ((1R,3S)- 3- amino- cyclo- pentyl) acetic acid | 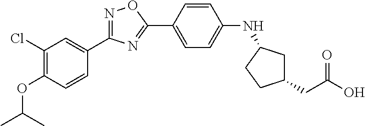 | 2-((1R,3S)- 3-(4-(3-(3- chloro-4- isopropoxy phenyl)- 1,2,4- oxadiazol- 5- yl)phenyl- amino) cyclo pentyl) acetic acid | 456.13 (M + H)⁺ | 3.04 (g) |
| D. 27 | 3- chloro- 4-iso- pro- poxy- benzo- nitrile | 4- fluoro- benzoyl chloride | (3S)- methyl 3- amino- 1- methyl cyclo- pentane- carboxy- late | 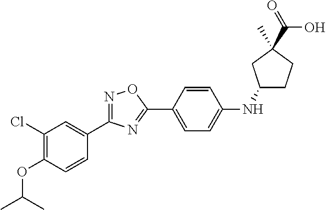 | (1S,3S)-3- (4-(3-(3- chloro-4- isopropoxy phenyl)- 1,2,4- oxadiazol- 5- yl)phenyl- amino)-1- methyl- cyclo- pentane- carboxylic acid | 456.13 (M + H)⁺ | 3.02 (g) |
| D. 28 | 3- chloro- 4-iso- pro- poxy- benzo- nitrile | 4- fluoro- benzoyl chloride | (3S)- methyl 3- amino- 1- methyl cyclo- pentane- carbo- xylate | 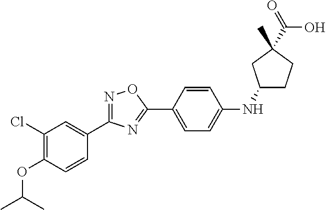 | (1R,3S)-3- (4-(3-(3- chloro-4- isopropoxy phenyl)- 1,2,4- oxadiazol- 5- yl)phenyl- amino)-1- methyl- cyclo- pentane- carboxylic acid | 456.13 (M + H)⁺ | 3.15 (g) |
| D. 29 | 3- chloro- 4-iso- pro- poxy- benzo- nitrile | 4- fluoro- benzoyl chloride | (3S)- methyl 3- amino- 1- fluoro- cyclo- pentane- carbo- xylate | 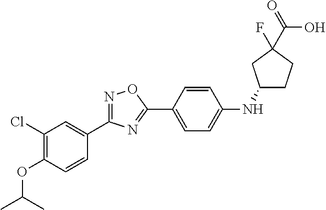 | (3S)-3-(4- (3-(3- chloro-4- isopropoxy pheny) 1,2,4- oxadiazol- 5- yl)phenyl- amino)-1- fluoro- cyclo- pentane- carboxylic acid | 460.11 (M + H)⁺ | 2.29 (g) |
| D. 30 | 3- chloro- 4-iso- pro- poxy- benzo- nitrile | 4- fluoro- benzoyl chloride | (3S)- methyl 3- amino- 1- hydroxy- cyclo- pentane- carbo- xylate | 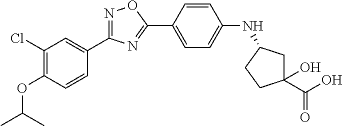 | (3S)-3-(4- (3-(3- chloro-4- isopropoxy phenyl)- 1,2,4- oxadiazol- 5- yl)phenyl- amino)-1- hydroxy- cyclo- pentane- carboxylic acid | 458.09 (M + H)⁺ | 2.35, 2.39 (g) |
| D. 31 | 3- chloro- 4-iso- pro- poxy- benzo- nitrile | 2- chloro- 4- fluoro- benzoyl chloride | (1R,3S)- 3- amino- cyclo- pentane- carbo- xylic acid | 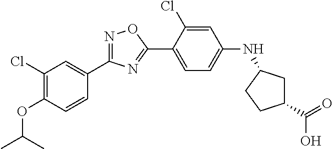 | (1R,3S)-3- (3-chloro- 4-(3-(3- chloro-4- isopropoxy phenyl)- 1,2,4 oxadiazol- 5- yl)phenyl- amino) cyclo- pentane- carboxylic acid | 476.14 (M + H)⁺ | 3.13 (g) |
| D. 32 | 3- bromo- 4-iso- pro- poxy- benzo- nitrile | 2- chloro- 4- fluoro- benzoic acid | (1R,3S)- 3- amino- cyclo- pentane- carbo- xylic acid | 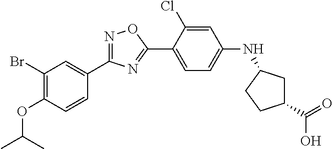 | (1R,3S)-3- (4-(3-(3- bromo-4- isopropoxy phenyl)- 1,2,4- oxadiazol- 5-yl)-3- chloro- phenyl- amino) cyclo- pentane carboxylic acid | 522.14 (M + H)⁺ | 3.17 (g) |
| D. 33 | 3- choro- 4-iso- pro- poxy- benzo- nitrile | 2- bromo- 4- fluoro- benzoic acid | (1R,3S)- 3- amino- cyclo- pentane- carbo- xylic acid | 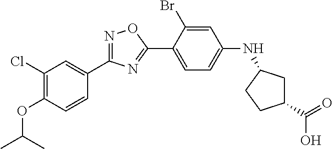 | (1R,3S)-3- (3-bromo- 4-(3-(3- chloro-4- isopropoxy phenyl)- 1,2,4- oxadiazol- 5- yl)phenyl- amino) cyclo- pentane- carboxylic acid | 522.14 (M + H)⁺ | 2.88 (h) |
| D. 34 | 3- chloro- 4-iso- pro- poxy- benzo- nitrile | 4- fluoro- 3- (tri- fluoro- methyl) benzoyl chloride | (1R,3S)- 3- amino- cyclo- pentane- carbo- xylic acid | 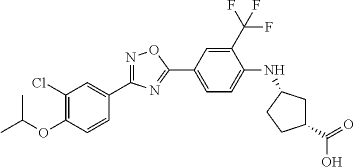 | (1R,3S)-3- (4-(3-(3- chloro-4- isopropoxy phenyl)- 1,2,4- oxadiazol- 5-yl)-2- (trifluoro- methyl) phenyl- amino) cyclo- pentane carboxylic acid | 508.13 (M − H)⁻ | 3.35 (g) |
| D. 35 | 4- Phenyl- 5- (trifluo- rometh- yl)thio- phene- 2- carboni- trile (Prepa- ration #82) | 4- Fluoro- benzoyl chloride | (1R,3S)- 3- amino- cyclo- pentane- carbo- xylic acid | 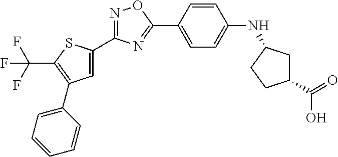 | (1R,3S)-3- (4-(3-(4- phenyl-5- (trifluoro- methyl) thiophen- 2-yl)- 1,2,4- oxadiazol- 5- yl)phenyl- amino) cyclo- pentane- carboxylic acid | 500.13 (M + H)⁺ | 3.22 (g) |
| D. 36 | (1aS,5a R)- 1,1,2- Tri- methyl- 1,1a,5, 5a- tetra- hydro- 3- thia- cyclo- propa[a] penta- lene-4- carbo- nitrile (Prepa- ration #83 | 4- Fluoro- benzoyl chloride | (1R,3S)- 3- amino- cyclo- pentane- carbo- xylic acid | 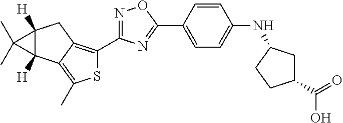 | (S)-3-{4- [3- ((1aS,5aR)- 1,1,2- Trimethyl- 1,1a,5,5a- tetrahydro- 3-thia- cyclopropa [a]pentalen- 4-yl)- [1,2,4]oxa- diazol-5-yl]- phenyl- amino}- cyclo- pentane- carboxylic acid | 450.15 (M + H)⁺ | 3.31 (g) |
| D. 37 | 5- chloro- 6-iso- pro- poxy- nico- tino- nitrile (Prepa- ration #85) | 4- Fluoro- 3- methyl benzoic acid | (1R,3S)- 3- amino- cyclo- pentane- carbo- xylic acid | 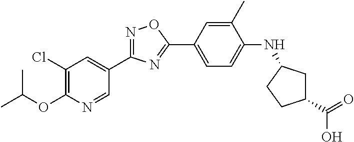 | (1R,3S)-3- (4-(3-(5- chloro-6- isopropoxy pyridin-3- yl)-1,2,4- oxadiazol- 5-yl)-2- methyl- phenyl- amino) cyclo- pentane carboxylic acid | 457.09, 459.09 (M + H)⁺ | 3.39 (g) |
| D. 38 | 3- chloro- 4-iso- pro- poxy- benzo- nitrile | 4- fluoro- 2- (tri- fluoro- methyl) benzoyl chloride | (1R,3S)- 3- amino- cyclo- pentane- carbo- xylic acid | 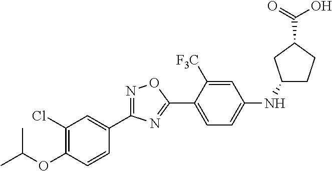 | (1R,3S)-3- (4-(3-(3- chloro-4- isopropoxy phenyl)- 1,2,4- oxadiazol- 5-yl)-3- (trifluoro- methyl) phenyl- amino) cyclo- pentane carboxylic acid | 510 (M + H)⁺ | 3.02 (g) |
| D. 39 | (Z)-5- chloro- N′- hy- droxy- 6-iso- pro- poxy- nico- tinimi- damide | 4- fluoro- 2- (tri- fluoro- methyl) benzoyl chloride | (1R,3S)- 3- amino- cyclo- pentane- carbo- xylic acid | 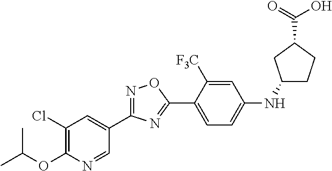 | (1R,3S)-3- (4-(3-(5- chloro-6- isopropoxy pyridin-3- yl)-1,2,4- oxadiazol- 5-yl)-3- (trifluoro- methyl) phenyl- amino) cyclo- pentane carboxylic acid | 511 (M + H)⁺ | 3.16 (g) |
| D. 40 | 3- chloro- 4-iso- pro- poxy- benzo- nitrile | 4- fluoro- 2- methyl benzoyl chloride | (1R,3S)- 3- amino- cyclo- pentane- carbo- xylic acid | 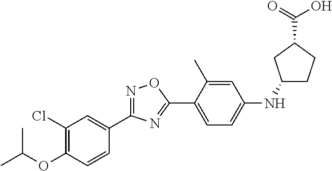 | (1R,3S)-3- (4-(3-(3- chloro-4- isopropoxy phenyl)- 1,2,4- oxadiazol- 5-yl)-3- methyl- phenyl- amino) cyclo- pentane carboxylic acid | 456 (M + H)⁺ | 3.05 (g) |
| D. 41 | 3- chloro- 4-iso- pro- poxy- benzo- nitrile | 4- fluoro- 3- methyl benzoyl chloride | (1R,3S)- 3- amino- cyclo- pen- tane- carbo- xylic acid | 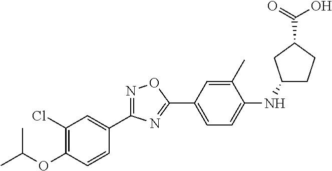 | (1R,3S)-3- (4-(3-(3- chloro-4- isopropoxy phenyl)- 1,2,4- oxadiazol- 5-yl)-2- methyl- phenyl- amino) cyclo- pentane carboxylic acid | 456 (M + H)⁺ | 3.12 (g) |
| D. 42 | 3- chloro- 4-iso- pro- poxy- benzo- nitrile | 4- fluoro- benzoyl chloride | 4- amino- 2- methyl buta- noic acid (Prepa- ration #79- 81) | 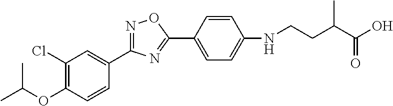 | 4-(4-(3-(3- chloro-4- isopropoxy phenyl)- 1,2,4- oxadiazol- 5- yl)phenyl- amino)-2- methyl- butanoic acid | 430 (M + H)⁺ | 2.91 (g) |
| D. 43 | 3- chloro- 4-iso- pro- poxy- benzo- nitrile | 4- fluoro- benzoyl chloride | glycine tert- butyl ester hydro- chloride | 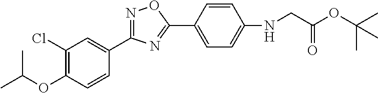 | tert-butyl 2-(4-(3-(3- chloro-4- isopropoxy phenyl)- 1,2,4- oxadiazol- 5-yl) phenyl- amino) acetate | 444 (M + H)⁺ | 2.98 (j) |
| D. 44 | 3- chloro- 4-iso- pro- poxy- benzo- nitrile | 4- fluoro- benoyl chloride | beta- alanine tert- butyl- ester hydro- chloride | 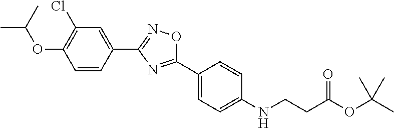 | tert-butyl 3-(4-(3-(3- chloro-4- isopropoxy phenyl)- 1,2,4- oxadiazol- 5- yl)phenyl- amino) propanoate | 458 (M + H)⁺ | 3.20 (j) |
| D. 45 | 3- chloro- 4-iso- pro- poxy- benzo- nitrile | 4- fluoro- benoyl chloride | (3- Amino- propyl)- phos- phonic acid diethyl ester | 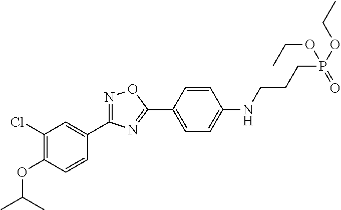 | (3-{4-[3- (3-Chloro- 4-iso- propoxy- phenyl)- [1,2,4]oxa- diazol-5-yl]- phenyl- amino}- propyl)- phosphonic acid diethyl ester | 508 (M + H)⁺ | 3.03 (b) |
| D. 46 | 3- chloro- 4-iso- pro- poxy- benzo- nitrile | 4- fluoro- benoyl chloride | (3- Amino- propyl)- phos- phonic acid diethyl ester | 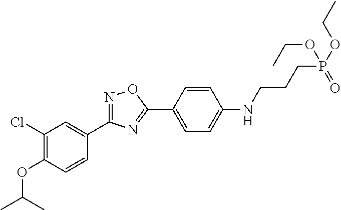 | (3-{4-[3- (3-Chloro- 4- iso- propoxy- phenyl)- [1,2,4] oxadiazol- 5-yl]- phenyl- amino}- propyl)- phosphonic acid diethyl ester | 508 (M + H)⁺ | 3.03 (b) |

TABLE E
Examples made using General Procedures R, K, D and V

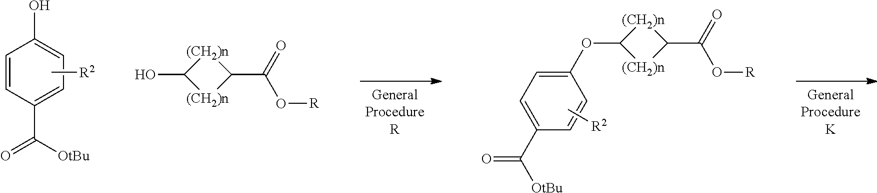

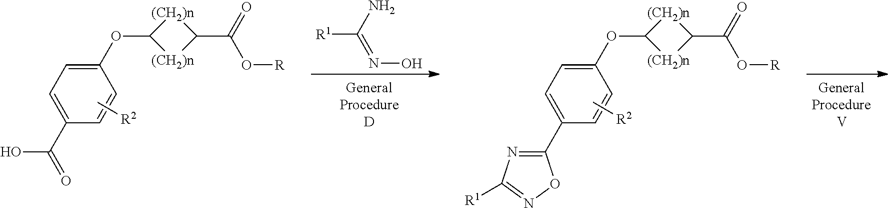

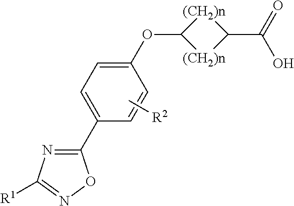

| | Nitrile | | | | | | Rₜ/min |
|---|---|---|---|---|---|---|---|
| Ex | pre- | | | | | | (me- |
| # | cursor | Phenol | Alcohol | Structure | Name | m/z | thod) |
| E.1 | 3- chloro- 4-iso- propo- xy- benzo- nitrile | benzyl 4- hydroxy- benzoate | 3- hydroxy- cyclo- butane- carbo- xylic acid tert- butyl ester | 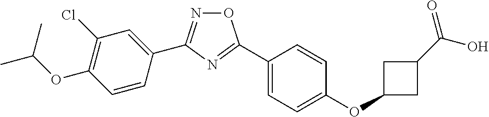 | 3-{4-[3-(3- chloro-4- iso- propoxy- phenyl)- [1,2,4] oxa- diazol- 5-yl]- phe- noxy}- cyclo- butane- carbo- xylic acid | 429.0 (M + H)⁺ | 2.63 (a) |

TABLE F
Examples made using General Procedures R, C, D and K

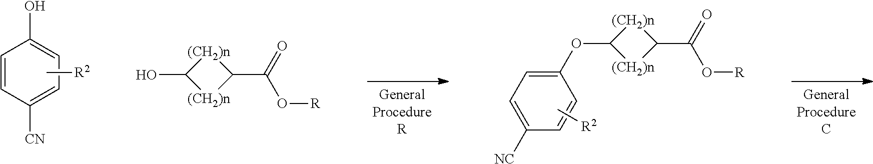

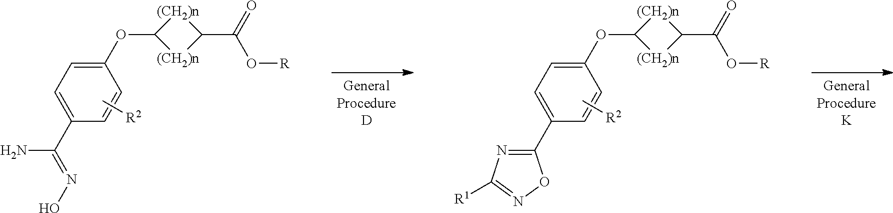

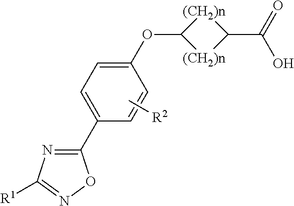

| Ex | Benzo- | | | | | Rₜ/min |
|---|---|---|---|---|---|---|
| # | nitrile | Alcohol | Structure | Name | m/z | (method) |
| F.1 | 2- Chloro- 4- hydro- xy- benzo- nitrile | 3- Hydroxy- cyclo- butane- carbo- xylic acid tert- butyl ester | 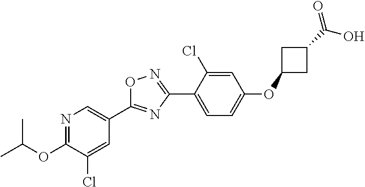 | 3-{3-Chloro-4- [5-(5-chloro-6- isopropoxy- pyridin-3- yl)- [1,2,4]oxadiazol- 3-yl]-phenoxy}- cyclobutane- carboxylic acid | 464 (M + H)⁺ | 2.72 (a) |
| F.2 | 2- Chloro- 4- hydroxy- benzo- nitrile | 4- Hydroxy- cyclo- hexane- carboxylic acid tert- butyl ester | 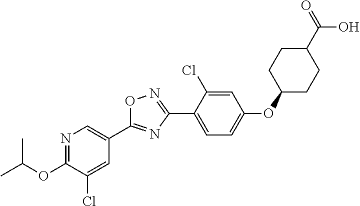 | 4-{3-Chloro-4- [5-(5-chloro-6- isopropoxy- pyridin-3- yl)- [1,2,4] oxadiazol- 3-yl]-phenoxyl- cyclohexane- carboxylic acid | 490 (M + H) | 2.83 (a) |

TABLE G
Examples made using General Procedures C, D or E, L and K

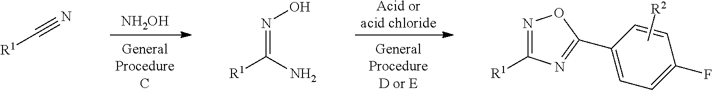

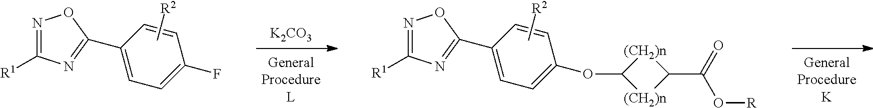

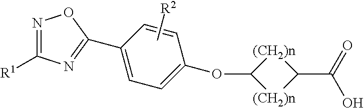

| | | | | | | | Rₜ/min |
|---|---|---|---|---|---|---|---|
| Ex | Nitrile | Acid | | | | | (me- |
| # | precursor | chloride | alcohol | Structure | Name | m/z | thod) |
| G.1 | 5- Chloro- 6-iso- pro- poxy- nico- tino- nitrile | 2- Chloro- 4- fluoro- benzoyl chloride | 3- Hy- droxy- cyclo- butane- carbo- xylic acid tert- butyl ester | 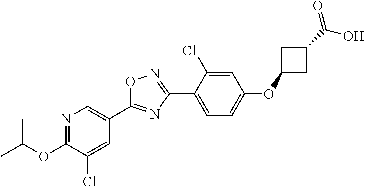 | 3-{3- Chloro- 4-[3-(5- chloro-6- isopro- poxy- pyridin- 3-yl)- [1,2,4] oxa- diazol- 5-yl]- phenoxy}- cyclo- butane- carboxylic acid | 464 (M − H)⁺ | 2.99 (a) |
| G.2 | 5- Chloro- 6- isopro- poxy- nico- tino- nitrile | 2- Chloro- 4- fluoro- benzoyl chloride | 3- Hy- droxy- cyclo- butane- carbo- xylic acid tert- butyl ester | 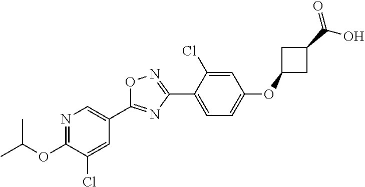 | 3-{3- Chloro- 4-[-(5- chloro-6- isopro- poxy pyridin- 3-yl)- [1,2,4] oxa- diazol- 5-yl]- phenoxy}- cyclo- butane- carboxylic acid | 464 (M − H)⁺ | 2.99 (a) |

TABLE H
Examples made using General Procedures P, C, D and K or Q

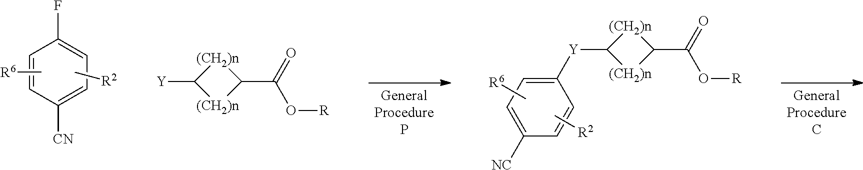

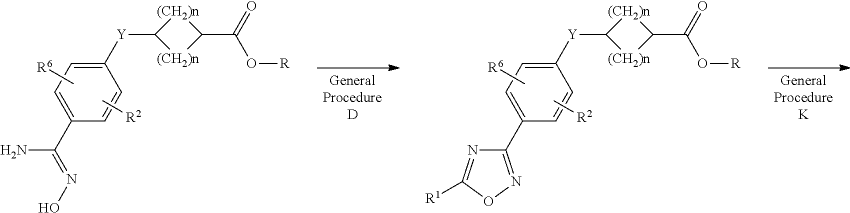

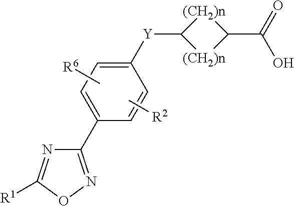

| | | Carbo- | | | | | |
|---|---|---|---|---|---|---|---|
| Ex | Benzo- | xylic | Alcohol | | | | Rₜ/min |
| # | nitrile | acid | or amine | Structure | Name | m/z | (method) |
| H.1 | 2- Chloro- 4- benzo- nitrile | 5- Chloro- 6- iso- propoxy- fluoro- nicotinic acid | 3- Hydroxy- cyclo- butane- carbo- xylic acid ethyl ester | 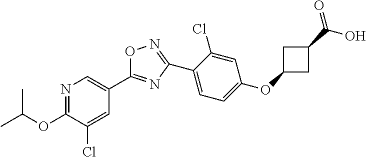 | 3-{3- Chloro-4- [5-(5- chloro-6- isopro- poxy- pyridin-3- yl)- [1,2,4] oxa- diazol- 3-yl]- phenoxy}- cyclobutane- carboxylic acid- phenoxy}- cyclobutane- carboxylic acid | 464 (M + H)⁺ | 2.86 (a) |
| H.2 | 2- Chloro- 4- hydroxy- benzo- nitrile | 3- Chloro- 4- isopro- poxy- benzoic acid | (1r,4r)- tert-butyl 4- hydroxy- cyclo- hexane- carbo- xylate | 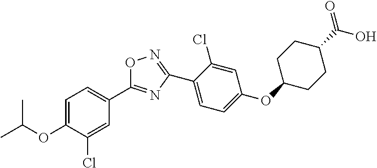 | (1r,4r)-4- (3-chloro- 4-(5-(3- chloro-4- isopropoxy phenyl)- 1,2,4- oxadiazol- 3- yl)phenoxy) cyclohexane- carboxylic acid | 491 (M + H) | 2.97 (b) |
| H.3 | 3,4- difluo- robenzo nitrile | 5- Chloro- 6 - isopro- poxy- nicotinic acid | (1r,4r)- tert-butyl 4- hydroxy- cyclo- hexane- carbo- xylate | 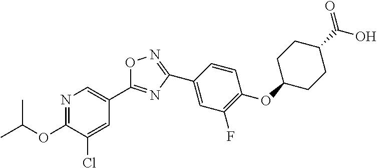 | (1r,4r)-4- (4-(5-(5- chloro -6- isopropoxy pyridin-3- yl)-1,2,4- oxadiazol- 3-yl)-2- fluoro- phenoxy) cyclo- hexane- carbo- xylic acid | 476 (M + H) | 2.81 (b) |
| H.4 | 3,4- difluo- robenzo nitrile | 5- Chloro- 6- iso- propoxy- nicotinic acid | (1s,4s)- tert-butyl 4- hydroxy- cyclo- hexane- carbo- xylate | 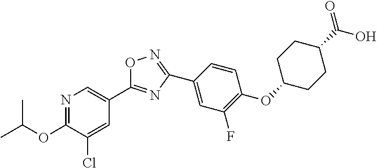 | (1s,4s)-4- (4-(5-(5- chloro-6- isopropoxy pyridin-3- yl)-1,2,4- oxadiazol- 3-yl)-2- fluorophe- noxy)cyclo- hexane- carboxylic acid | 476 (M + H) | 2.80 (b) |
| H.5 | 4- fluoro- benzo- nitrile | 5- Chloro- 6- iso- propoxy- nicotinic acid | (1r,4r)- tert-butyl 4- hydroxy- cyclo- hexane- carbo- xylate | 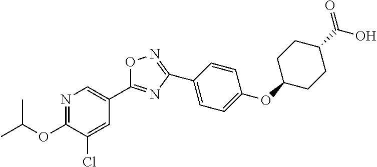 | (1r,4r)-4- (4-(5-(5- chloro-6- isopropoxy pyridin-3- yl)-1,2,4- oxadiazol- 3- yl)phenoxy) cyclo- hexane- carboxylic acid | 458 (M + H) | 2.78 (b) |
| H.6 | 4- fluoro- benzo- nitrile | 5- Chloro- 6- isopro- poxy- nicotinic acid | (1R,3S)- 3- Amino- cyclo- pentane- carbo- xylic acid | 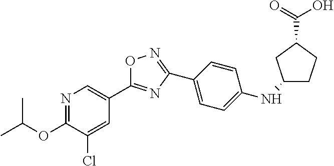 | (1R,3S)-3- (4-(5-(5- chloro-6- isopropoxy pyridin-3- yl)-1,2,4- oxadiazol- 3- yl)phenyl- amino) cyclo- pentane- carboxylic acid | 443 (M + H) | 2.81 (b) |
| H.7 | 4- fluoro- benzo- nitrile Note H | 5- Chloro- 6- isopro- poxy- nicotinic acid | (1R,3S)- 3- Amino- cyclo- pentane- carbo- xylic acid | 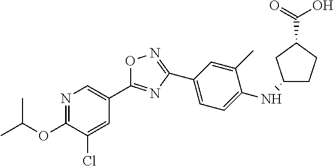 | (1R,3S)-3- (4-(5-(5- chloro-6- isopropoxy pyridin-3- yl)-1,2,4- oxadiazol- 3-yl)-2- methyl- phenyl- amino) cyclo- pentane carboxylic acid | 457 (M + H) | 2.94 (b) |
| H.8 | 4- fluoro- benzo- nitrile Note H | 3- Chloro- 4- isopro- poxy- benzoic acid | (1R,3S)- 3- Amino- cyclo- pentane- carbo- xylic acid | 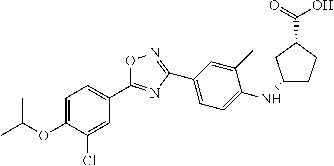 | (1R,3S)-3- (4-(5-(3- chloro-4- isopropoxy phenyl)- 1,2,4- oxadiazol- 3-yl)-2- methyl- phenyl- amino) cyclo- pentane carboxylic acid | 457 (M + H) | 2.87 (b) |

Note H: See Preparation of Additional Examples section for modification of benzonitrile intermediate

TABLE I
Examples made using General Procedures C, D or E, P, K, R and K or Q

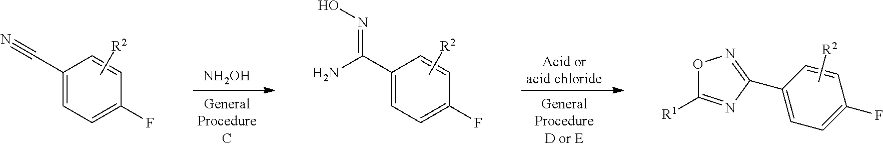

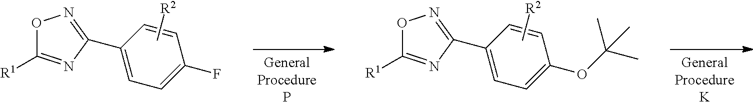

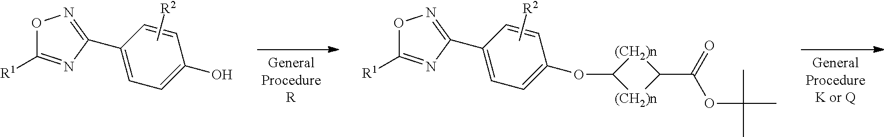

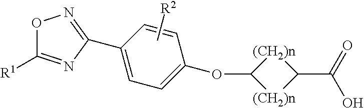

| Ex | Nitrile | | | | | | Rₜ/min |
|---|---|---|---|---|---|---|---|
| # | precursor | Acid | alcohol | Structure | Name | m/z | (method) |
| I.1 | 4- Fluoro 2- chloro- benzo- nitril | 5- Chloro- 6- isopro- poxy- nicotinic acid | 4- Hydroxy- cyclo- hexane- carbo- xylic acid ethyl ester | 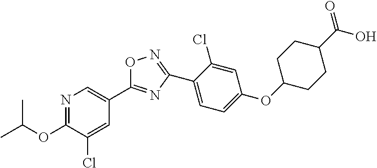 | 4-{3- Chloro- 4-[5-(5- chloro-6- isopro- poxy- pyridin- 3-yl)- [1,2,4] oxa- diazol- 3-yl]- phenoxy}- cyclo- hexane- carbo- xylic acid | 492 (M − H)⁺ | 3.26 + 3.31 (a) Note F |
| I.2 | 4- Fluoro- 2- chloro- benzo- nitril | 5- Chloro- 6- isopro- poxy- nicotinic acid | 3- Hydroxy- cyclo- pentane- carbo- xylic acid ethyl ester | 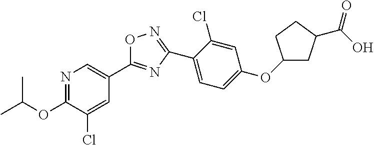 | 3-{3- Chloro- 4-[5-(5- chloro-6- isopro- poxy- pyridin- 3-yl)- [1,2,4] oxa- diazol- 3-yl]- phenoxy}- cyclo- pentane- carbo- xylic acid | 478 (M − H)⁺ | 3.18 + 3.25 (a) Note F. |
| I.3 | 4- Fluoro- 2- chloro- benzo- nitril | 5- Chloro- 6- oxy- nicotinic acid | 3- Hydroxy- cyclo- hexane- carbo- xylic acid ethyl ester | 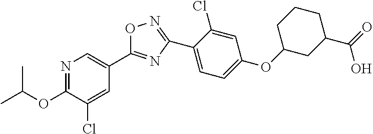 | 3-{3- Chloro- 4-[5-(5- chloro-6- isopro- poxy- pyridin- 3-yl)- isoprop [1,2,4] oxa- diazol- 3-yl]- phenoxy}- cyclo- hexane- carbo- xylic acid | 478 (M − H)⁺ | 3.31 + 3.38 (a) Note F. |
| I.4 | 4 Fluoro- 2- chloro- benzo- nitril | 5- Chloro- 6- isopro- poxy- nicotinic acid | 4- Hydroxy- cyclo- hexane- carbo- xylic acid tert- butyl ester | 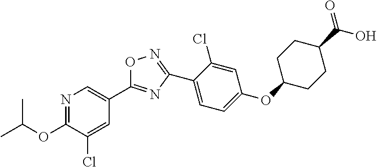 | 4-{3- Chloro- 4-[5-(5- chloro-6- isopro- poxy- pyridin- 3-yl)- [1,2,4] oxa- diazol- 3-yl]- phenoxy}- cyclo- hexane- carbo- xylic acid | 492 (M − H)⁺ | 3.12 (a) |
| I.5 | 4- Fluoro- 2- chloro- benzo- nitril | 5- Chloro- 6- isopro- poxy- nico- tinic acid | 3- Hydroxy- cyclo- pentane- carbo- xylic acid ethyl ester | 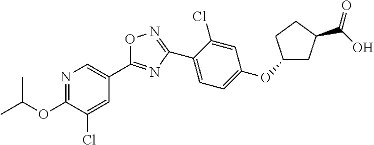 | (1R,3R)- 3-{3- Chloro- 4-{5-[5- chloro- 6- isopro- poxy- pyridin- 3-yl)- [1,2,4] oxa- diazol- 3-yl]- phenoxy}- cyclo- pentane- carbo- xylic acid | 478 (M − H)⁺ | 2.78 (a) Note G |
| I.6 | 4- Fluoro- 2- chloro- benzo- nitril | 5- Chloro- 6- iso- propoxy- nicotinic acid | 3- Hydroxy- cyclo- pentane- carbo- xylic acid ethyl ester | 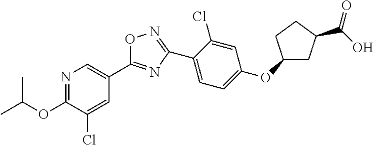 | (1R,3S)- 3-{3- Chloro- 4-[5-(5- chloro- 6- isopro- poxy- pyridin- 3-yl)- [1,2,4] oxa- diazol- 3-yl]- phenoxy}- cyclo- pentane- carbo- xylic acid | 478 (M − H)⁺ | 2.74 (a) Note G |
| I.7 | 4- Fluoro- 2- chloro- benzo- nitril | 5- Chloro- 6- isopro- poxy- nicotinic acid | 3- Hydroxy- cyclo- pentane- carbo- xylic acid ethyl ester | 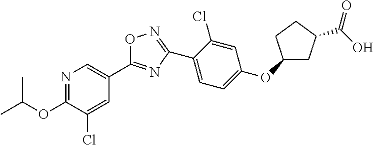 | (1S,3S)- 3-{3- Chloro- 4-[5-(5- chloro- 6- isopro- poxy- pyridin- 3-yl)- [1,2,4] oxa- diazol- 3-yl]- phenoxy}- cyclo- pentane- carbo- xylic acid | 478 (M − H)⁺ | 2.78 (a) Note G |
| I.8 | 4- Fluoro- 2- chloro- benzo- nitril | 5- Chloro- 6- isopro- poxy- nicotinic acid | 3- Hydroxy- cyclo- pentane- carbo- xylic acid ethyl ester | 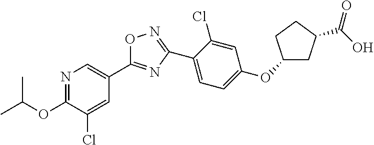 | (1S,3R)- 3-{3- Chloro- 4-[5-(5- chloro- 6- isopro- poxy- pyridin- 3-yl)- [1,2,4] oxa- diazol- 3-yl]- phenoxy}- cyclo- pentane- carbo- xylic acid | 478 (M − H)⁺ | 2.74 (a) Note G |
| I.9 | 2- Chloro 4- fluoro- benzo- nitrile | 5- Chloro- 6- deutero- isopro- poxy- nicotinic acid | 3- Hydroxy- cyclo- butane- carbo- xylic acid tert- butyl ester | 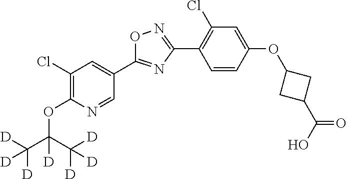 | 3-{3- Chloro- 4-[5-(5- chloro-6- deutero- isopro- poxy- pyridin- 3-yl)- [1,2,4] oxa- diazol- 3-yl]- phenoxy}- cyclo- butane- carbo- xylic acidphen- oxy}- cyclo- butane- carbo- xylic acid | — | 21.717 min, 98.8% and 21.138 min, 1.19% (r) |

Note F: Mixture of isomers
Note G: Crude mixture of 4 isomers was separated using two orthogonal preparative chromatography methods.
Method A: Isocratic elution using 0.12% trifluoroacetic acid in heptane (solvent B) and isopropanol (solvent A) in a ratio of 20% solvent A:80% solvent B on a Daicel ADH column (20 × 250 mm) over 16.5 min, monitoring with UV (280 nm).
Method B: Gradient elution using 0.12% trifluoroacetic acid in heptane (solvent B) and 1:1/ethanol:methanol (solvent A), 20-60% solvent A on a Daicel ADH column (20 × 250 mm) over 19.5 minutes, monitoring with UV (280 nm).

TABLE J
Examples made using General Procedures C, D or E, I or J and Q

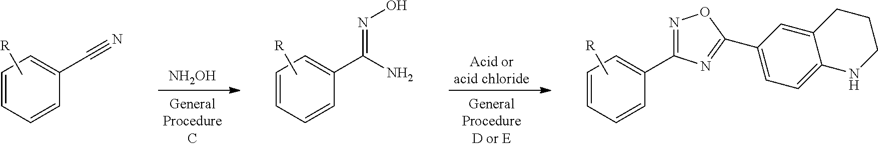

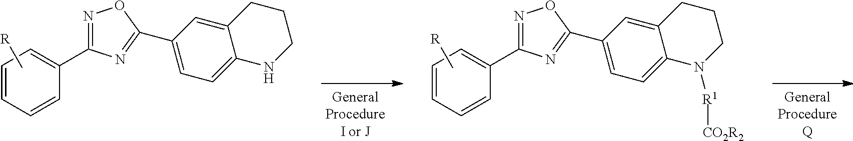

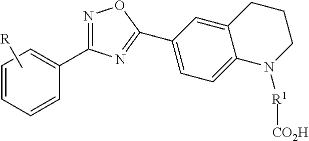

| | | Acid or | | | | | Rₜ/min |
|---|---|---|---|---|---|---|---|
| Ex | Nitrile | acid | | | | | (me- |
| # | precursor | chloride | ester | Structure | Name | m/z | thod) |
| J.1 | 3-chloro- 4- isopropo- xybenzo- nitrile | 4- fluoro- benzoyl chloride | ethyl 2- bromo- acetate | 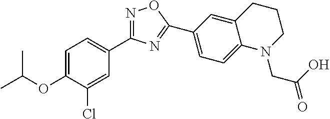 | 2-(6-(3-(3- chloro-4- isopropoxy- phenyl)-1,2,4- oxadiazol-5- yl)-3,4- dihydroquinolin- 1(2H)- yl)acetic acid | 428 (M + H)⁺ | 2.55 (a) |
| J.2 | 3-chloro- 4- isopropo- xybenzo- nitrile | 4- fluoro- benzoyl chloride | ethyl 3- bromo- pro- panoate | 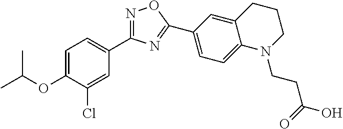 | 3-(6-(3-(3- chloro-4- isopropoxy- phenyl)-1,2,4- oxadiazol-5- yl)-3,4- dihydroquinolin- 1(2H)- yl)propanoic acid | 443 (M + H)⁺ | 2.78 (a) |

TABLE K
Examples made using Preparations #61, 62 and 63
The letter in parentheses below the phenol precursors indicates the General Procedure by
which the phenol precursor was made.

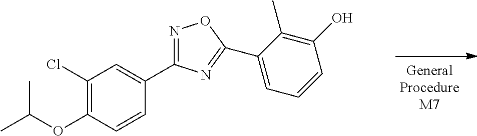

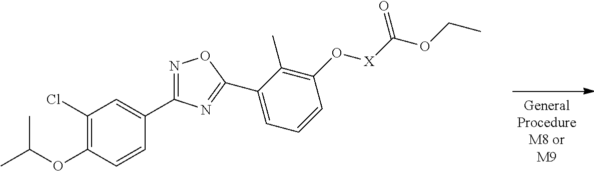

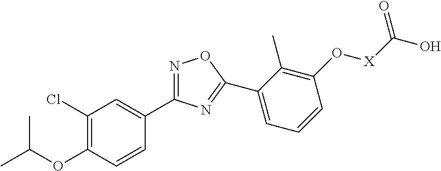

| | Phenol | Alkyl | | | Rₜ/min | |
|---|---|---|---|---|---|---|
| Ex # | precursor | Halide | Product | Name | (method) | m/z |
| K.1 | 3-(3-(3- chloro-4- isopropoxy phenyl)- 1,2,4- oxadiazol- 5-yl)-2- methyl- phenol (A,C, E) | (E)-ethyl 4- bromobut- 2-enoate | 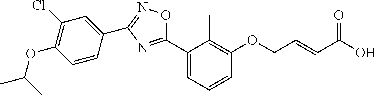 | (E)-4-(3-(3-(3- chloro-4- isopropoxy- phenyl)-1,2,4- oxadiazol-5- yl)-2- methylphenoxy) but-2-enoic acid | 3.05 (g) | 429 (M + H)⁺ |
| K.2 | 3-(3-(3- chloro-4- isopropoxy phenyl)- 1,2,4- oxadiazol- 5-yl)-2- methyl- phenol (A, C, E) | (E)-ethyl 4- bromobut- 2-enoate | 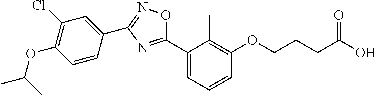 | 4-(3-(3-(3- chloro-4- isopropoxy- phenyl)-1,2,4- oxadiazol-5- 5-yl)-2- methyl- phenoxy) butanoic acid | 3.08 (g) | 431 (M + H)⁺ |
| K.3 | 3-(3-(3- chloro-4- isopropoxy phenyl)- 1,2,4- oxadiazol- 5-yl)-2- methyl- phenol (A, C, E) | (E)-ethyl 4- bromobut- 2-enoate | 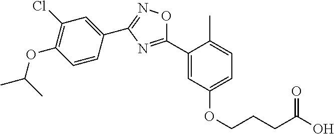 | 4-(3-(3-(3- chloro-4- isopropoxy- phenyl)-1,2,4- oxadiazol-5- yl)-4- methyl- phenoxy) butanoic acid | 3.11 (g) | 431 (M + H)⁺ |

TABLE L
Examples made using General Procedures C and D or E

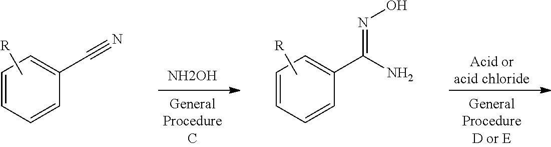

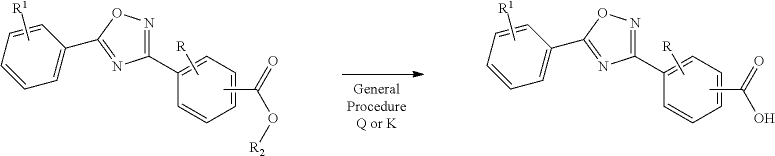

| | Carboxylic | | | | | |
|---|---|---|---|---|---|---|
| | acid | | | | | Rₜ/min |
| Ex # | precursor | Nitrile | Structure | Name | m/z | (method) |
| L.1 | 3- chloro- 4 isopro- poxy- benzoic acid carbo- xylate | (1R,3S)- methyl 3- (4- cyano- phenyl- amino) cyclo- pentane (Pro- cedures #40 and 41) | 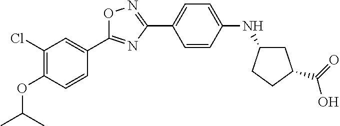 | (1R,3S)-3-(4-(5- (3-chloro-4- isopropoxy- phenyl)-1,2,4- oxadiazol-3- yl)phenylamino) cyclopentane- carboxylic acid | 442.27 (M + H)⁺ | 2.94 (g) |

TABLE M
Examples prepared using General Procedure S

| | | | Rₜ min | |
|---|---|---|---|---|
| | | | (Table 2, | m/z ESI+ |
| Phosphonate ester | Product | Example # | Method) | (M + H)⁺ |
| diethyl 3-(4-(3-(3- chloro-4- isopropoxyphenyl)- 1,2,4-oxadiazol-5- yl)benzylamino)propyl phosphonate (prepared in table B entry 17,) | 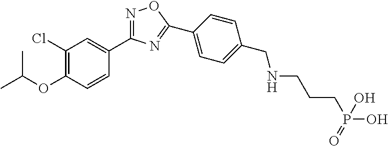 | M.1 | 1.85 (b) | 466 |
| diethyl (1S,3R)-3-(4- (3-(3-chloro-4- isopropoxyphenyl)- 1,2,4-oxadiazol-5- yl)phenylamino)cyclo- pentylphosphonate (Preparation #68) | 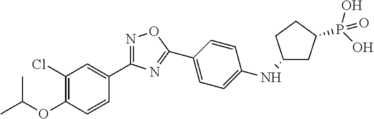 | M.2 | 2.01 (b) | 478 |
| diethyl (1R,3R)-3-(4- (3-(3-chloro-4- isopropoxyphenyl)- 1,2,4-oxadiazol-5- yl)phenylamino)cyclo- pentylphosphonate (Preparation #68) | 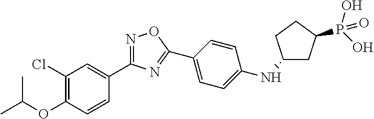 | M.3 | 1.97 (b) | 478 |
| diethyl (1R,3R)-3-(4- (3-(3-chloro-4- isopropoxyphenyl)- 1,2,4-oxadiazol-5- yl)phenylamino)cyclo- pentylphosphonate (Preparation #68) | 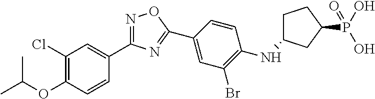 | M.4 | 2.18 (b) | 556 |
| diethyl (1R,3S)-3-(4- (3-(3-chloro-4- isopropoxyphenyl)- 1,2,4-oxadiazol-5- yl)phenylamino)cyclo- pentylphosphonate (Preparation #68) | 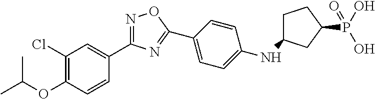 | M.5 | 1.96 (b) | 478 |
| diethyl (1R,35)-3-(4- (3-(3-chloro-4- isopropoxyphenyl)- 1,2,4-oxadiazol-5- yl)phenylamino)cyclo- pentylphosphonate (Preparation #68) | 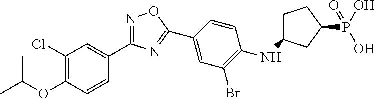 | M.6 | 2.16 (b) | 556 |
| diethyl (1S,3S)-3-(4- (3-(3-chloro-4- isopropoxyphenyl)- 1,2,4-oxadiazol-5- yl)phenylamino)cyclo- pentylphosphonate (Preparation #68) | 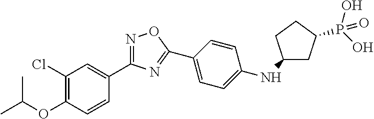 | M.7 | 1.97 (b) | 478 |

TABLE N
Examples made using General Procedures T, U and V

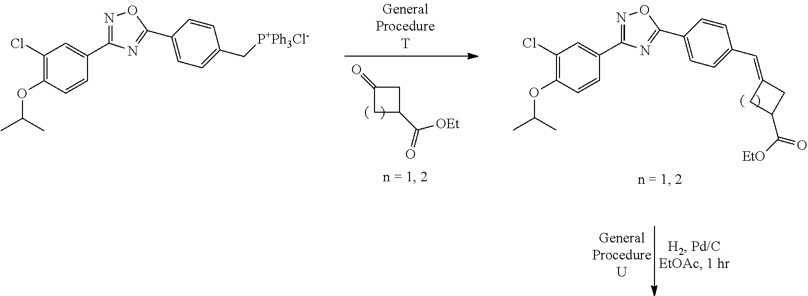

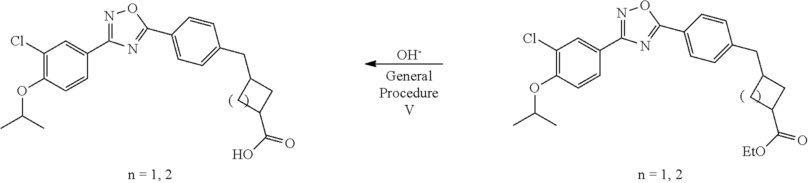

| | | Acid or | | | | | |
|---|---|---|---|---|---|---|---|
| Ex | Nitrile | acid | | | | | Rₜ/min |
| # | precursor | chloride | Ketone | Structure | Name | m/z | (method) |
| N.1 | 3- chloro- 4- isopro- poxy- benzo- nitrile | 4- (chloro methyl) benzoyl chloride | ethyl 3- oxo- cyclo- butane- carbo- xylate | 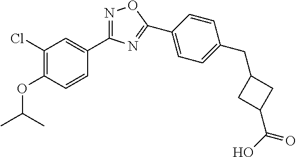 | 3-(4-(3-(3- chloro-4- isopropoxy phenyl) 1,2,4- oxadiazol- 5- yl)benzyl) cyclobutane- carboxylic acid | 427.24 (M + H)⁺ | 3.13 (g) |
| N.2 | 3- chloro- 4- isopro- poxy- benzo- nitrile | 4- (chloro methyl) benzoyl chloride | ethyl 3- oxo- cyclo- pentane- carbo- xylate | 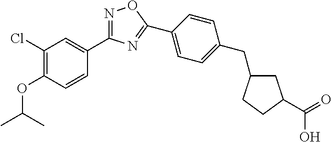 | 3-(4-(3-(3- chloro-4- isopropoxy phenyl)- 1,2,4- oxadiazol- 5- yl)benzyl) cyclopentane- carboxylic acid | 441.11 (M + H)⁺ | 3.32 (g) |

TABLE P
The following deuterated compounds could be prepared by those skilled in the art:

| | | Acid or | | | |
|---|---|---|---|---|---|
| Ex | Nitrile | acid | | | |
| # | precursor | chloride | Amine | Structure | Name |
| P.1 | 3-chloro- 4- isopropo- xybenzo- nitrile | 4- cyano- benzoyl chloride | pyrro- lidine-3- carboxylic acid | 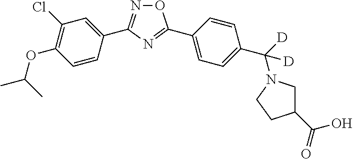 | Deutero-1-(4- (3-(3-chloro-4- isopropoxy- phenyl)-1,2,4- oxadiazol-5- yl)benzyl) pyrrolidine-3- carboxylic acid |
| P.2 | 5- Chloro- 6- isopropoxy- nicotino- nitrile | 4-Fluoro-2- methyl- benzoyl chloride | (1R,3S)- 3- amino- cyclopen- tane- carboxylic acid | 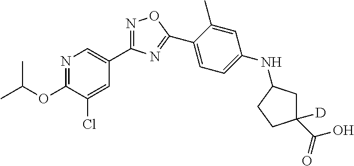 | Deutero- (1R,3S)-3-{4- [3-(5-Chloro-6- isopropoxy- pyridin-3- yl)- [1,2,4]oxadiazol- 5-yl]-3-methyl- phenylamino}- cyclopentane- carboxylic acid |
| P.3 | 3-chloro- 4- isopropoxy- benzo- nitrile | benzyl 4- hydroxy- benzoate | 3- hydroxy- cyclo- butane- carboxylic acid tert- butyl ester | 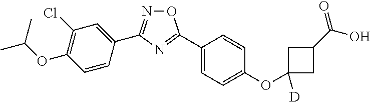 | Deutero-3-{4- [3-(3-chloro-4- isopropoxy- phenyl)- [1,2,4]oxadiazol- 5-yl}- phenoxy}- cyclobutane- carboxylic acid |
| P.4 | 3-chloro- 4- isopropoxy- benzo- nitrile | 4- (chloro- methyl) benzoyl chloride | ethyl 3- oxocyclo- butane- carbo- xylate | 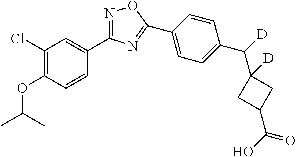 | Deutero-3-(4- (3-(3-chloro-4- isopropoxy- phenyl)- 1,2,4- oxadiazol-5- yl)benzyl)cyclo- butanecarboxylic acid |

### Preparation of Additional Examples

### Preparation #1

### Preparation of 3-chloro-4-isopropoxy-benzoic acid

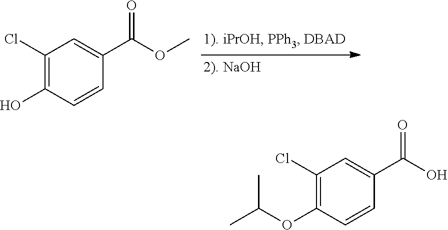

Into a round bottom flask was added triphenylphosphine (62 g, 0.263 mol), 3-chloro-4-hydroxy-benzoic acid methyl ester (10 g, 0.0535 mol) and anhydrous THF (500 mL). The mixture was briefly stirred under nitrogen, then DBAD (19.75 g, 0.0858 mol) was added. The mixture was stirred for a few minutes before adding anhydrous isopropanol (5.125 mL, 0.067 mol). After the reaction mixture was stirred at RT under an atmosphere of nitrogen for about 3 h, DBAD (19.75 g, 0.0858 mol) and anhydrous isopropanol (5.125 mL, 0.067 mol) were added and the mixture was left to stir at RT overnight. The solvent was removed under reduced pressure. The residue was dissolved in a minimum amount of ethyl acetate. Heptane was added and the precipitate was removed by filtration. The filtrate was brought up in methanol. Water was added until the solution was cloudy. The precipitate was filtered off The methanol/water precipitation procedure was repeated two more times. The filtrate was taken up in THF (200 mL) and 5 M NaOH (200 mL). The mixture was stirred at RT overnight. The organic solvent was removed under reduced pressure. The aqueous layer was extracted three times with ethyl acetate. The aqueous layer was further acidified to pH 1-2 with 2 M HCl. The cloudy suspension was then extracted with ethyl acetate three times. The organic layers were combined, dried over magnesium sulfate, and concentrated to dryness to give 3-chloro-4-isopropoxy-benzoic acid (8.4 g, 71.4%) as a white solid.

LC/MS (Table 1, Method b) Rₜ=2.42 min, m/z (M−H)⁻ 213; ¹H NMR (400 MHz, DMSO-d₆) 612.95 (s, 1H), 7.87 (m, 2H), 7.25 (d, 1H), 4.79 (m, 1H), 1.32 (d, 6H)

### Preparation #2

### 4-(3-(3-chloro-4-isopropoxyphenyl)-1,2,4-oxadiazol-5-yl)benzonitrile

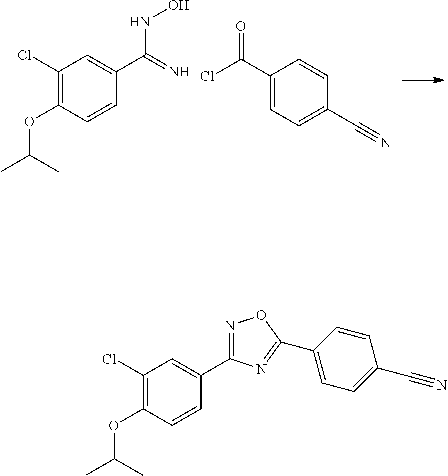

3-Chloro-N-hydroxy-4-isopropoxybenzimidamide (10 g, 43.7 mmol) was dissolved in DMF (219 mL) under nitrogen. The mixture was heated at about 110° C. for about 10 min. A solution of 4-cyanobenzoyl chloride (7.24 g, 43.7 mmol) dissolved in DMF (30 mL) was added dropwise over about 20 min and the reaction heated at about 110° C. for about 4 h until LC/MS showed the reaction was complete. The reaction was cooled in an ice bath and poured into rapidly stirred water (1000 mL). The resulting white precipitate was collected by vacuum filtration and washed with water. The precipitate was dissolved in methylene chloride and washed with 1 N HCl and then brine. The methylene chloride was dried over sodium sulfate, filtered, and evaporated. Heptane and DCM were added to the residue and the mixture heated until the DCM had boiled off after which the mixture was allowed to cool. Solids did not dissolve in hot heptane. The resulting solid was collected by vacuum filtration and washed with heptane to provide 4-(3-(3-chloro-4-isopropoxyphenyl)-1,2,4-oxadiazol-5-yl)benzonitrile (12.568 g, 37.0 mmol, 85% yield) as a tan solid: LC/MS (Table 1, Method a) Rₜ=4.58 min.; MS m/z: 340.20 (M+H)⁺.

### Preparation #3

### 4-(3-(3-chloro-4-isopropoxyphenyl)-1,2,4-oxadiazol-5-yl)benzaldehyde

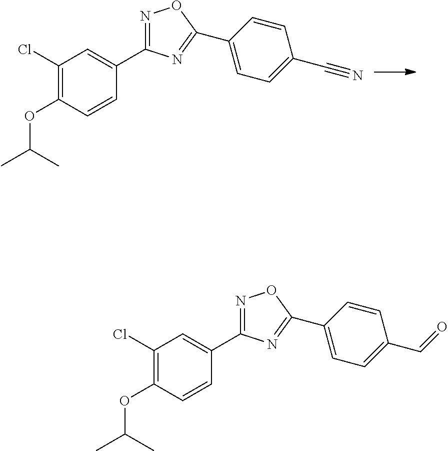

4-(3-(3-Chloro-4-isopropoxyphenyl)-1,2,4-oxadiazol-5-yl)benzonitrile (10 g, 29.4 mmol) was dissolved in dichloromethane (535 mL) under nitrogen. The reaction was cooled to about −40° C. in a dry ice/ACN bath measuring the temperature internally. A solution of Dibal-H (58.9 mL, 58.9 mmol) was added dropwise and the reaction stirred for about 30 min. and then quenched with methanol. The mixture was stirred until the bubbles subsided. The mixture was then warmed to RT and stirred rapidly with a 10% solution of Rochelle's salt. The separated layers were extracted with DCM (3×100 mL). The combined extracts were stirred rapidly with about 100 mL of 1 N HCl and the solution turned from orange to colorless. TLC indicated the mixture had been cleaned up to just one spot with some baseline material. The layers were separated and the aqueous layer extracted with DCM (2×100 mL). The combined organic extracts were washed with brine, dried over sodium sulfate, filtered, and evaporated to dryness to afford an off white solid. The solid was stirred with heptane and the solvent removed carefully via pipette. The solid was dried under vacuum to afford 4-(3-(3-chloro-4-isopropoxyphenyl)-1,2,4-oxadiazol-5-yl)benzaldehyde (9.15 g, 26.7 mmol, 91% yield) as white solid: LC/MS (Table 1, Method a) Rₜ=4.59 min.; MS m/z: 343.26, 345.18 (M+H)⁺.

### Example #1

### 1-(4-(3-(3-chloro-4-isopropoxyphenyl)-1,2,4-oxadiazol-5-yl)benzyl)azetidine-3-carboxylic acid

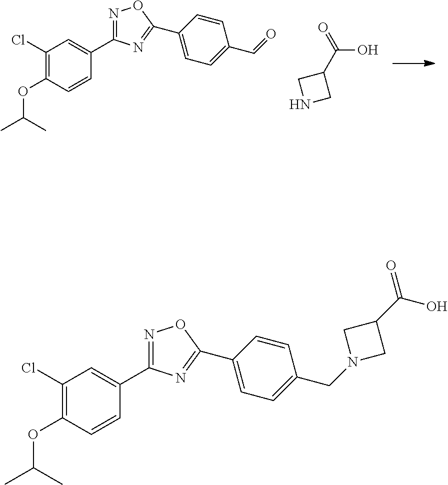

Azetidine-3-carboxylic acid (3.72 g, 36.8 mmol) (Synchem) was dissolved in acetic acid (16.03 mL, 280 mmol) and methanol (2 mL). This was added to a stirred suspension of 4-(3-(3-chloro-4-isopropoxyphenyl)-1,2,4-oxadiazol-5-yl)benzaldehyde (12 g, 35.0 mmol) in MeOH (600 mL). The reaction was stirred for about 18 h. Sodium cyanoborohydride (5.50 g, 88 mmol) was added and the reaction stirred for about 4 h. The reaction was cooled with an ice bath and the precipitate was collected by vacuum filtration and washed with ice cold methanol and then ether. TLC showed impurities still present. The crude solid was dissolved in 1:1 EtOAc/(6:3:1 CHCl₃/MeOH/NH₄OH) with a little extra added NH₄OH. Chromatography over silica gel in a mixture of 1:1 EtOAc/(6:3:1 CHCl₃/MeOH/NH₄OH) increasing to all 6:3:1 CHCl₃/MeOH/NH₄OH eluted the product. The fractions were evaporated to dryness to afford a colorless film/oil. The mixture was dissolved in methanol and evaporated to dryness. The residue was resuspended in the minimum amount of methanol, water was added and the mixture filtered, washed with water and then ether. The residue was dried under vacuum at ambient temperature and then under vacuum at about 60° C. to remove trace methanol to afford 1-(4-(3-(3-chloro-4-isopropoxyphenyl)-1,2,4-oxadiazol-5-yl)benzyl)azetidine-3-carboxylic acid (8.3 g, 19.40 mmol, 55.4% yield) as a white solid: LC/MS (Table 1, Method a) Rₜ=2.94 min.; MS m/z: 428.31, 430.27 (M+H)⁺; mp 194.8-195.9° C.; ¹H NMR (400 MHz, DMSO-d) δ ppm 8.12 (d, J=8.34 Hz, 2H), 8.06 (d, J=2.13 Hz, 1H), 8.00 (dd, J=8.67, 2.15 Hz, 1H), 7.54 (d, J=8.36 Hz, 2H), 7.39 (d, J=9.06 Hz, 1H), 4.88-4.77 (m, 1H), 3.67 (s, 2H), 3.48-3.38 (m, 2H), 3.29-3.19 (m, 3H), 1.35 (d, J=6.02 Hz, 6H).

### Preparation #4

### Preparation of 3-(3-chloro-4-isopropoxyphenyl)-5-(4-fluorophenyl)-1,2,4-oxadiazole

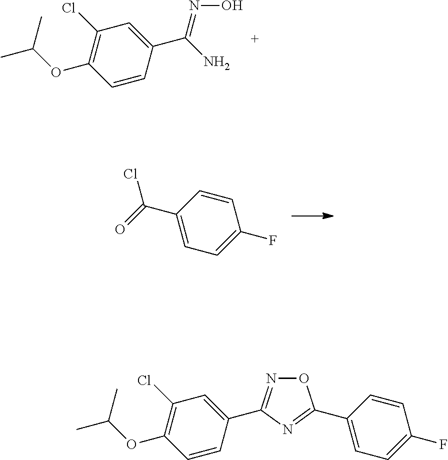

(Z)-3-Chloro-N′-hydroxy-4-isopropoxybenzimidamide (2.0 g. 8.75 mmol), 4-fluorobenzoyl chloride (2.1 g, 13.12 mmol) and pyridine (12 mL) are loaded into a 20 mL microwave vial equipped with a stir bar. The vessel is sealed and the reaction heated to about 200° C. with cooling for about 25 min. The mixture was purified using normal phase chromatography to afford a pale brown solid. Analysis by LC/MS showed this to be a 35:30:21 mixture of 3-(3-chloro-4-isopropoxyphenyl)-5-(4-fluorophenyl)-1,2,4-oxadiazole, 2-chloro-4-(5-(4-fluorophenyl)-1,2,4-oxadiazol-3-yl)phenol and 4-fluorobenzoic acid. The mixture was purified a second time using normal phase chromatography to afford 5 fractions. Fractions 1, 2 and 3 were combined and evaporated to dryness to afford 3-(3-chloro-4-isopropoxyphenyl)-5-(4-fluorophenyl)-1,2,4-oxadiazole (420 mmol, 14%) as a white solid. LC/MS (Table 1, Method a) Rₜ=2.85 min, m/z 333.10 (M−H)⁺

### Preparation #5

### Preparation of 3-chloro-4-(3-(3-chloro-4-isopropoxyphenyl)-1,2,4-oxadiazol-5-yl)benzonitrile

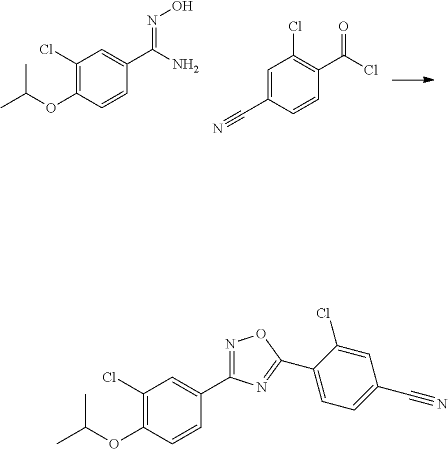

To a 250 mL round bottom flask equipped with a stir bar and charged with 2-chloro-4-cyanobenzoic acid (3.0 g, 16.52 mmol), was added anhydrous DCM (80 mL), and DMF (0.064 mL, 0.826 mmol). Oxalyl chloride (8.26 mL, 16.52 mmol) (2M solution in DCM) was then added slowly and the mixture was stirred under nitrogen at ambient temperature. Upon addition of the oxalyl chloride, gas evolution began and the suspended solid began to dissolve. After about 2-3 h, the reaction became translucent. The mixture was concentrated in vacuo. The resulting crude material was dissolved in pyridine (50 mL). To this was added (Z)-3-chloro-N′-hydroxy-4-isopropoxybenzimidamide (1.258 g, 5.50 mmol). The mixture was heated to about 100° C. under an atmosphere of nitrogen for about 16 h. The resulting mixture was cooled to ambient temperature. Pyridine was removed under reduced pressure and the resulting material was triturated in DCM and MeOH mixture (about 1:1). The resulting precipitate was left standing for a few minutes at ambient temperature then was collected via filtration, washed with a mixture of 1:1 DCM/MeOH, and then with straight MeOH and dried in a vacuum oven for about 48 h to yield 3-chloro-4-(3-(3-chloro-4-isopropoxyphenyl)-1,2,4-oxadiazol-5-yl)benzonitrile (1.529 g, 4.09 mmol, 25%) as a beige solid. ¹H NMR (400 MHz, DMSO) δ ppm 8.39 (d, J=1.53 Hz, 1H), 8.35 (d, J=8.15 Hz, 1H), 8.09 (dd, J=8.14, 1.53 Hz, 1H), 8.05 (d, J=2.11 Hz, 1H), 8.00 (dd, J=8.63, 2.12 Hz, 1H), 7.39 (d, J=8.82 Hz, 1H), 4.82 (sept, J=6.04 Hz, 1H), 1.35 (d, J=6.01 Hz, 6H).

### Example #2

### Preparation of 2-(4-(3-(3-chloro-4-isopropoxyphenyl)-1,2,4-oxadiazol-5-yl)phenyl)propan-2-amine

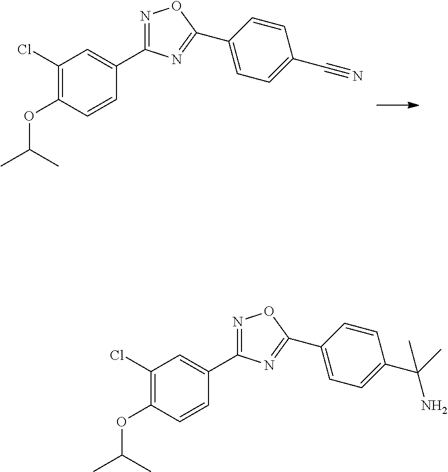

Anhydrous cerium (III) chloride (5.57 g, 22.60 mmol) and anhydrous tetrahydrofuran (20 mL) were added to a dry 2-neck round bottom flask under nitrogen. The resulting suspension was sonicated for a few minutes and then stirred at room temperature for about 90 minutes. The mixture was then cooled to about −50° C., and methylithium (14.13 mL, 22.60 mmol) was added slowly. After about 60 min, and warming to about 0° C., the reaction was cooled to about −50° C. and 4-(3-(3-chloro-4-isopropoxyphenyl)-1,2,4-oxadiazol-5-yl)benzonitrile (2.4 g, 7.06 mmol) in 8 mL of anhydrous THF was added drop-wise, to keep the temperature of the reaction at about −50° C. The reaction was maintained at about −50° C. for about 1 h, then left to warm to RT overnight. The next day the reaction was cooled to about −50° C., and quenched by the addition of 21 mL of 35% NH₄OH. The quenched reaction was left to warm to RT over about two h. The mixture was filtered through Celite® and washed with DCM (4×60 mL). The filtrate was collected and then washed with water and dried over MgSO₄. Solvent was removed under reduced pressure and the crude material was purified by RP-HPLC (A=50 mM ammonium acetate, B=ACN; 30-70% B over 30.0 min (21.0 mL/min flow rate); 21.2×250 mm Thermo Hyperprep C18 column, 8 μm particles) to give 2-(4-(3-(3-chloro-4-isopropoxyphenyl)-1,2,4-oxadiazol-5-yl)phenyl)propan-2-amine as the acetic acid salt (309 mg; 10.1%). LC/MS (Table 1, Method a) Rₜ=2.61 min; ¹H NMR (400 MHz, DMSO-d6) δ ppm 8.14-7.94 (m, 4H), 7.80 (d, J=8.43 Hz, 2H), 7.37 (d, J=8.81 Hz, 1H), 4.80 (sept, J=6.04 Hz, 1H), 1.85 (s, 3H), 1.39 (s, 6H), 1.36-1.31 (d, J=6.04 Hz, 6H)

### Example #3

### Preparation of methyl 3-(2-(4-(3-(3-chloro-4-isopropoxyphenyl)-1,2,4-oxadiazol-5-yl)phenyl)propan-2-ylamino)propanoate

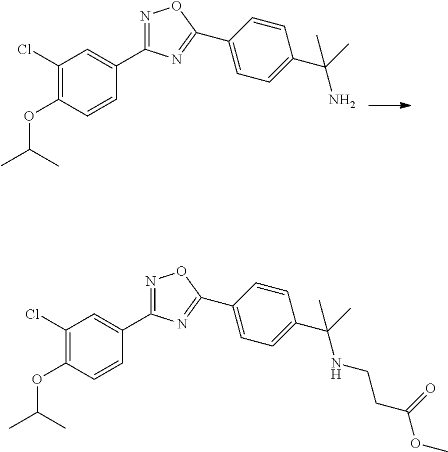

2-(4-(3-(3-Chloro-4-isopropoxyphenyl)-1,2,4-oxadiazol-5-yl)phenyl)propan-2-amine and acetic acid (132 mg, 0.306 mmol) was added to a 5 mL microwave vial equipped with a stirring bar. Methyl acrylate (52.6 mg, 0.611 mmol), and MeOH (3.0 mL) were added, the vial capped, and the reaction heated to about 120° C. for about 90 min under microwave irradiation (Biotage Optimizer™, 300 W). After about 90 min another aliquot of methyl acrylate (52.6 mg, 0.611 mmol) was added and the reaction heated for about another 60 min at about 120° C. The reaction was cooled and the solvent removed under reduced pressure. The crude material was purified by RP-HPLC (A=50 mM ammonium acetate, B=ACN; 30-70% B over 30.0 min (21.0 mL/min flow rate); 21.2×250 mm Thermo Hyperprep C18 column, 8 μm particles) to give methyl 3-(2-(4-(3-(3-chloro-4-isopropoxyphenyl)-1,2,4-oxadiazol-5-yl)phenyl)propan-2-ylamino)propanoate (83.5 mg; 59.7%). LC/MS (Table 1, Method a) Rₜ=2.78 min, m/z=458.29 (M=H)⁺.

### Example #4

### Preparation of 3-(2-(4-(3-(3-chloro-4-isopropoxyphenyl)-1,2,4-oxadiazol-5-yl)phenyl)propan-2-ylamino)propanoic acid

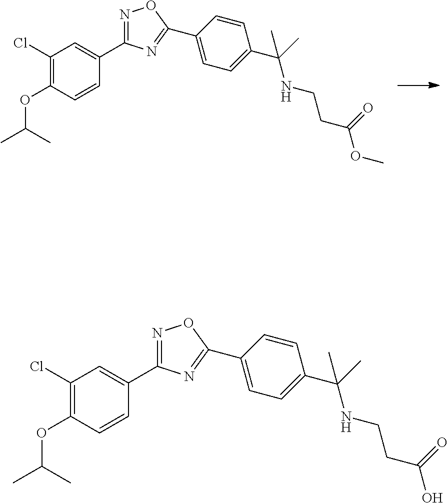

Methyl 3-(2-(4-(3-(3-chloro-4-isopropoxyphenyl)-1,2,4-oxadiazol-5-yl)phenyl)propan-2-ylamino)propanoate (83 mg, 0.181 mmol) was dissolved in ethanol (4 mL) and NaOH (4 mL, 8.00 mmol) was added. The mixture was stirred at RT under nitrogen. After about 20 min the reaction was neutralized by drop-wise addition of acetic acid. The aqueous mixture was then frozen and lyophilized. DCM was added to the solid, filtered, and washed with DCM. The filtrate was concentrated and ether added to afford a slightly cloudy solution. 1N HCl in ether was added dropwise until white precipitate formed. The material was collected by filtration, washed with ether, and dried in a vacuum oven to give 3-(2-(4-(3-(3-chloro-4-isopropoxyphenyl)-1,2,4-oxadiazol-5-yl)phenyl)propan-2-ylamino)propanoic acid as the hydrochloric acid salt (61.5 mg; 70.6%). LC/MS (Table 1, Method a) Rₜ=1.98 min, m/z=444.29 (M=H)⁺; 1H NMR (400 MHz, DMSO-d6) δ ppm 8.32 (d, J=8.57 Hz, 2H), 8.12 (d, J=2.08 Hz, 1H), 8.03 (dd, J=8.64, 2.10 Hz, 1H), 7.85 (d, J=8.59 Hz, 2H), 7.25 (d, J=8.78 Hz, 1H), 4.79 (sept, J=6.11 Hz, 1H), 2.95 (t, J=6.20 Hz, 2H), 2.44 (t, J=6.17 Hz, 2H), 1.84 (s, 6H), 1.40 (d, J=6.04 Hz, 6H).

### Example #5

### Preparation of 3-(3-chloro-4-isopropoxyphenyl)-5-(1H-indol-4-yl)-1,2,4-oxadiazole

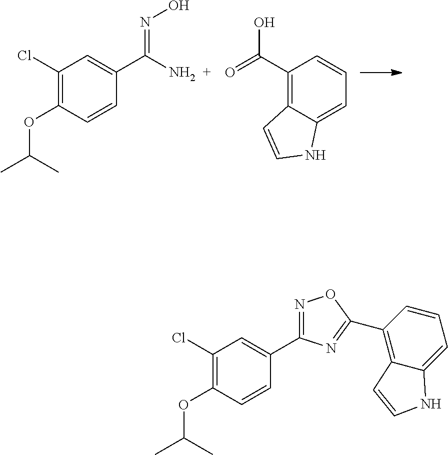

Under an atmosphere of nitrogen a mixture of 1H-indole-4-carboxylic acid (3.88 g, 24.05 mmol), (3-dimethylamino-propyl)-ethyl-carbodiimide hydrochloride (4.61 g, 24.05 mmol) and benzotriazol-1-ol hydrate (3.68 g, 24.05 mmol) in anhydrous DMF (61.4 mL) was stirred at ambient temperature for about 1 h. To the reaction mixture a solution of 3-chloro-N-hydroxy-4-isopropoxybenzamidine (5.0 g, 21.87 mmol) in DMF (11.51 mL) was added. The mixture was stirred and heated at about 140° C. for about 2 h. The mixture was cooled to ambient temperature and poured into water (1 L). The product was partitioned between ethyl acetate and the aqueous phase. The organic layer washed with 1N HCl (4×150 mL), 1N NaOH (2×150 mL) and water (2×300 mL), dried over MgSO₄ and filtered. The solvent was removed under reduced pressure and the crude product was purified by elution through Florisil with heptane/ethyl acetate (2:1) to give 3-(3-chloro-4-isopropoxyphenyl)-5-(1H-indol-4-yl)-1,2,4-oxadiazole (2.76 g, 35.7%). LC/MS (Table 1, Method b) Rₜ=2.69 min, m/z 354.17 (M+H)⁺.

### Preparation #6

### Preparation of (4-(3-(3-chloro-4-isopropoxyphenyl)-1,2,4-oxadiazol-5-yl)phenyl)methanol

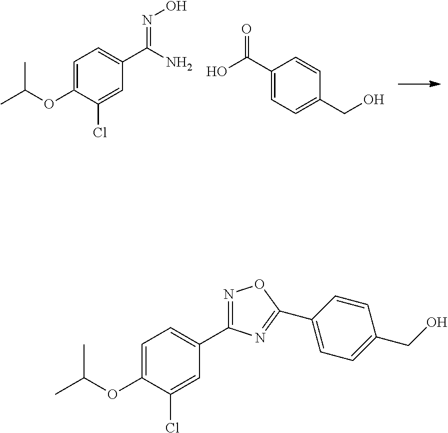

To a slurry of 4-(hydroxymethyl)benzoic acid (0.220 g, 1.443 mmol) in DMF (1.640 mL) was added EDC (0.277 g, 1.443 mmol) followed by HOBT hydrate (0.195 g, 1.443 mmol). After about 45 min. a solution of (Z)-3-chloro-N′-hydroxy-4-isopropoxybenzimidamide (0.300 g, 1.31 mmol) in DMF (1.640 mL) was added and the reaction mixture was heated to about 140° C. for about 2 h. After cooling to RT the reaction mixture was concentrated in vacuo and purified by chromatography on silica gel (eluting with EtOAc/Hep) to provide (4-(3-(3-chloro-4-isopropoxyphenyl)-1,2,4-oxadiazol-5-yl)phenyl)methanol (0.336 g, 71%) as an off-white solid. LC/MS (Table 1, Method c) Rₜ=2.80 min, m/z 345 (M+H)⁺.

### Preparation #7

### Preparation of 5-(4-(azidomethyl)phenyl)-3-(3-chloro-4-isopropoxyphenyl)-1,2,4-oxadiazole

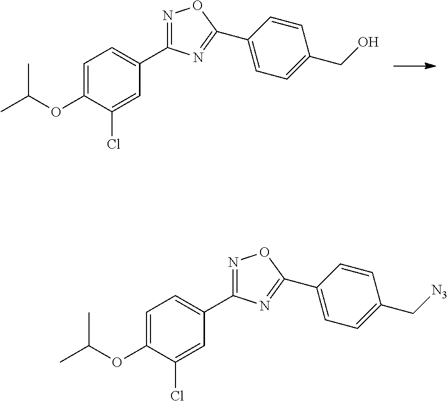

To a solution of (4-(3-(3-chloro-4-isopropoxyphenyl)-1,2,4-oxadiazol-5-yl)phenyl)methanol (0.100 g, 0.290 mmol) in THF (1.5 mL) was added DBU (0.048 mL, 0.319 mmol) followed by diphenyl phosphorazidate (0.069 mL, 0.319 mmol). After about 15 h the reaction mixture was poured into ether and saturated NaHCO₃. The organic layer was separated, washed with brine, dried (MgSO₄), concentrated in vacuo and purified by chromatography on silica gel (eluting with EtOAc/Hep) to provide 5-(4-(azidomethyl)phenyl)-3-(3-chloro-4-isopropoxyphenyl)-1,2,4-oxadiazole (0.066 g, 60%) as a colorless solid. LC/MS (Table 1, Method c) Rₜ=3.22 min, m/z 370 (M+H)⁺

### Example #6

### Preparation of (4-(3-(3-chloro-4-isopropoxyphenyl)-1,2,4-oxadiazol-5-yl)phenyl)methanamine

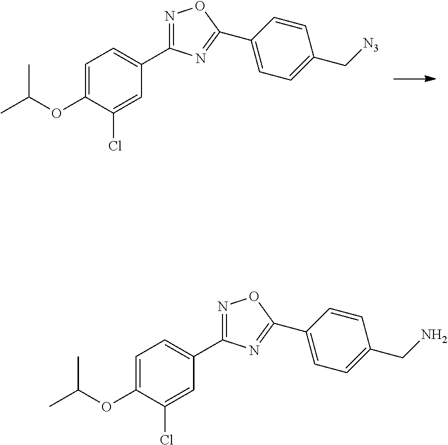

To a solution of 5-(4-(azidomethyl)phenyl)-3-(3-chloro-4-isopropoxyphenyl)-1,2,4-oxadiazole (0.066 g, 0.178 mmol) in THF (3.40 mL) and water (0.170 mL) was added polymer-supported triphenylphosphine (0.237 g, 0.711 mmol). After about 2 h the reaction mixture was heated to about 60° C. After about 1 h the reaction mixture was cooled to RT, filtered, concentrated in vacuo and purified by chromatography on silica gel (eluting with MeOH:DCM) to provide (4-(3-(3-chloro-4-isopropoxyphenyl)-1,2,4-oxadiazol-5-yl)phenyl)methanamine (40 mg, 64%) as a colorless solid.

LC/MS (Table 1, Method c) Rₜ=1.97 min, m/z 344 (M+H)⁺.

### Preparation #8

### 3-(3-(3-chloro-4-isopropoxyphenyl)-1,2,4-oxadiazol-5-yl)cyclopentanone

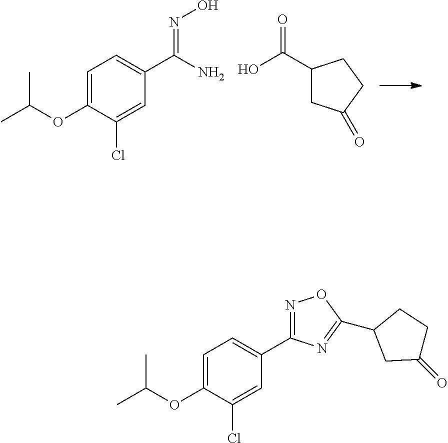

To a slurry of 3-oxocyclopentanecarboxylic acid (0.123 g, 0.962 mmol) in DMF (1.0 mL) was added EDC (0.184 g, 0.962 mmol) followed by HOBT hydrate (0.130 g, 0.962 mmol). After about 1 h a solution of (Z)-3-chloro-N′-hydroxy-4-isopropoxybenzimidamide (0.2 g, 0.875 mmol) in DMF (0.5 mL) was added and the reaction mixture was heated to about 140° C. for about 45 min. After cooling to RT the reaction mixture was concentrated in vacuo and purified by chromatography on silica gel (eluting with EtOAc/Hep) to provide 3-(3-(3-chloro-4-isopropoxyphenyl)-1,2,4-oxadiazol-5-yl)cyclopentanone (0.156 g, 56%) as a yellow oil.

LC/MS (Table 1, Method c) Rₜ=2.75 min, m/z 321 (M+H)⁺.

### Example #7

### 3-(3-(3-(3-chloro-4-isopropoxyphenyl)-1,2,4-oxadiazol-5-yl)cyclopentylamino)propanoic acid

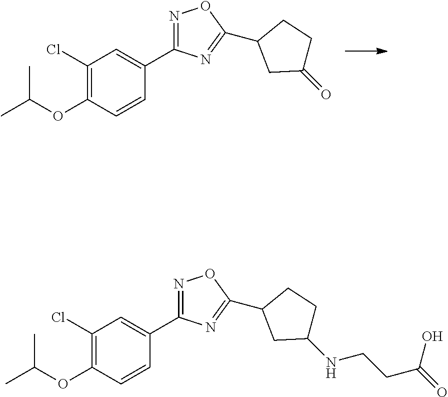

To a slurry of 3-(3-(3-chloro-4-isopropoxyphenyl)-1,2,4-oxadiazol-5-yl)cyclopentanone (0.178 g, 0.555 mmol) in MeOH (6.94 mL) and DCE (6.94 mL) was added acetic acid (0.254 mL, 4.44 mmol) followed by 3-aminopropanoic acid (0.494 g, 5.55 mmol). After about 1 h sodium cyanoborohydride (0.017 g, 0.277 mmol) was added to the reaction mixture. After about 15 h the reaction mixture was filtered, rinsing with MeOH. The filtrate was concentrated in vacuo and purified by RP HPLC to provide 3-(3-(3-(3-chloro-4-isopropoxyphenyl)-1,2,4-oxadiazol-5-yl)cyclopentylamino)propanoic acid. LC/MS (Table 1, Method c) Rₜ=1.64 min, m/z 394 (M+H)⁺.

### Example #8

### 4-(3-(3-(3-chloro-4-isopropoxyphenyl)-1,2,4-oxadiazol-5-yl)cyclopentylamino)butanoic acid

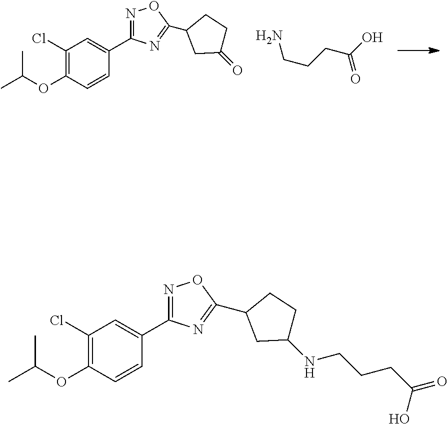

3-(3-(3-chloro-4-isopropoxyphenyl)-1,2,4-oxadiazol-5-yl)cyclopentanone (0.078 g, 0.243 mmol) was suspended in mixture of MeOH (3.04 mL) and DCE (3.04 mL). To this was added acetic acid (0.111 mL, 1.945 mmol) followed by 4-aminobutanoic acid (0.251 g, 2.432 mmol) as solid. The solution was stirred at RT for 0.5-1 h. Sodium cyanoborohydride (7.64 mg, 0.122 mmol) was then added in one portion. The reaction was stirred at RT overnight and LC/MS indicated reaction was complete. The excess amino acid was filtered off and the filtrate concentrated in vacuo. The crude oil was partitioned between ethyl acetate and brine. The organic layer was dried (MgSO₄) and concentrated to afford a residue that was purified on a Prep HPLC system using 30-100% ACN in 50 mM NH4OAc buffer at 21 mL/min. Fractions 12-14 were combined and concentrated in vacuo. The resulting material was sonicated in MeOH. The suspended precipitate was filtered, rinsed with cold MeOH and dried to 4-(3-(3-(3-chloro-4-isopropoxyphenyl)-1,2,4-oxadiazol-5-yield yl)cyclopentylamino)butanoic acid, (11 mg, 0.025 mmol) as white solid. LC/MS (Table 1, Method c) Rₜ=1.72 min, m/z 408.22 (M−H)⁺. 1H NMR (400 MHz, DMSO) δ ppm 8.06-7.94 (d, 2H), 7.89-7.79 (dd, J=1.99, 8.66 Hz, 1H), 7.14-7.06 (d, J=8.68 HZ, 1H), 4.78-4.65 (td, J=6.08, 12.13 Hz, 1H), 4.09-3.96 (dd, J=5.94, 10.14 Hz, 1H), 3.91-3.79 (m, 1H), 3.38-3.24 (t, J=7.26 Hz, 2H), 2.73-2.65 (dd, J=4.81 11.44 Hz, 2H), 2.65-2.56 (m, 1H), 2.53-2.37 (m, 2H), 2.37-2.28 (m, 1H), 2.28-2.22 (m, 1H), 2.22-2.20 (s, 1H), 2.20-2.10 (m, 2H), [2.10-1.96 (m, 1H) and 1.48-1.38 (d, J=6.05 Hz, 6H)

### Preparation #9

### (R)-benzyl 4-((2,2-dimethyl-1,3-dioxolan-4-yl)methoxy)benzoate

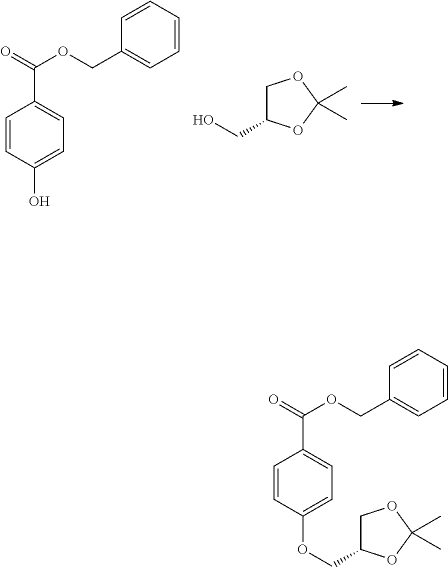

In a 250 mL round bottom flask was added triphenylphosphine (6.54 g, 24.92 mmol) in THF (79 mL) to give a colorless clear solution. The solution was cooled to about 0° C. by ice-bath. After stirring for about 15 min, diisopropyl azodicarboxylate (5.11 mL, 24.96 mmol) (orange liquid) was added dropwise over about 5 min. The reaction mixture turned into off-white suspension in the process. The reaction mixture was stirred at about 0° C. for about 30 min. Then a colorless solution of benzyl 4-hydroxybenzoate (5.69 g, 24.92 mmol) and (R)-(2,2-dimethyl-1,3-dioxolan-4-yl)methanol (3.00 mL, 23.73 mmol) in THF (39.5 mL) was added to the mixture, keeping the temperature at or below about 0° C. The solution turned clear light yellow. The solution was stirred for about 2 h at about 0° C. then slowly warmed to ambient temperature and stirred over the weekend. The mixture was concentrated in vacuo to give crude yellow oil (˜27 g). The crude oil was dissolved in ether. Then heptane was added. The resultant precipitate was sonicated and filtered. The filtrate was concentrated and purified via Analogix® system using RediSep™ RS 120 g column, with a gradient of 0-20% EtOAc/Heptane over 10 min at 50 mL/min then hold at 20% ethyl acetate for 20 min. Fractions containing product were combined and concentrated to afford (R)-benzyl 4-((2,2-dimethyl-1,3-dioxolan-4-yl)methoxy)benzoate as white solid (6.17 g, 23.73 mmol). LC/MS (Table 1, Method c) Rₜ=2.89 min, m/z 343.20 (M+H)⁺

### Preparation #10

### (R)-4-((2,2-dimethyl-1,3-dioxolan-4-yl)methoxy)benzoic acid

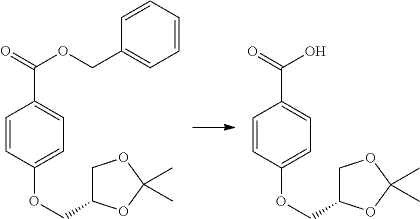

A 500 mL high-pressure flask was charged with palladium on carbon (0.300 g, 0.282 mmol), then MeOH (200 mL) was added, followed by (R)-benzyl 4-((2,2-dimethyl-1,3-dioxolan-4-yl)methoxy)benzoate (6.17 g, 18.02 mmol). The resulting suspension was allowed to shake under an atmosphere of hydrogen (35 Psi) at ambient temperature for about 2 h. The mixture was filtered through Celite® and the colorless filtrate was concentrated to afford (R)-4-((2,2-dimethyl-1,3-dioxolan-4-yl)methoxy)benzoic acid as white solid (4.45 g, 17.64 mmol).

LC/MS (Table 1, Method c) Rₜ=2.15 min, m/z 253.14 (M+H)⁺

### Preparation #11

### (R)-3-(3-chloro-4-isopropoxyphenyl)-5-(4-((2,2-dimethyl-1,3-dioxolan-4-yl)methoxy)phenyl)-1,2,4-oxadiazole

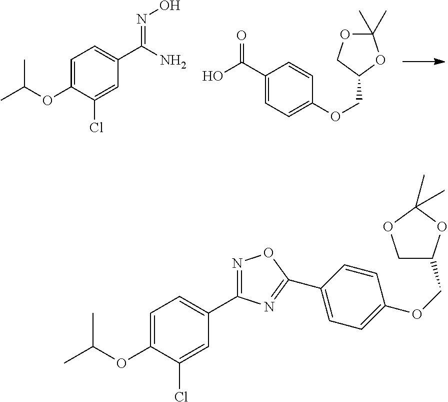

To a slurry of (R)-4-((2,2-dimethyl-1,3-dioxolan-4-yl)methoxy)benzoic acid (0.303 g, 1.203 mmol) in DMF (1.367 mL) was added EDC (0.231 g, 1.203 mmol) followed by HOBT hydrate (0.163 g, 1.203 mmol). After about 1.5 h a solution of (Z)-3-chloro-N′-hydroxy-4-isopropoxybenzimidamide (0.250 g, 1.09 mmol) in DMF (1.367 mL) was added. The reaction mixture was heated to about 140° C. for about 2 h. After cooling to RT the reaction mixture was concentrated in vacuo and purified by chromatography on silica gel (eluting with EtOAc/Hep) to provide (R)-3-(3-chloro-4-isopropoxyphenyl)-5-(4((2,2-dimethyl-1,3-dioxolan-4-yl)methoxy)phenyl)-1,2,4-oxadiazole (0.339 g, 70%) as a colorless solid. LC/MS (Table 1, Method c) Rₜ=3.36 min, m/z 445 (M+H)⁺.

### Example #9

### Preparation of (S)-3-(4-(3-(3-chloro-4-isopropoxyphenyl)-1,2,4-oxadiazol-5-yl)phenoxy)propane-1,2-diol

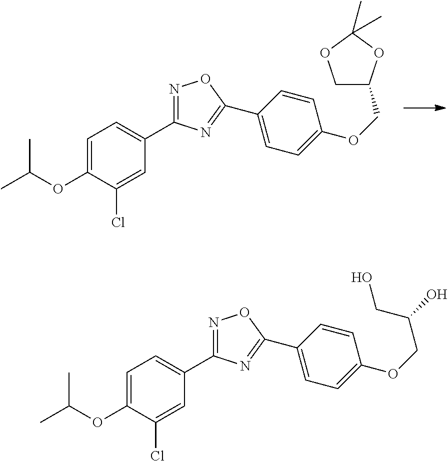

To a solution of (R)-3-(3-chloro-4-isopropoxyphenyl)-5-(4-((2,2-dimethyl-1,3-dioxolan-4-yl)methoxy)phenyl)-1,2,4-oxadiazole (0.339 g, 0.762 mmol) in THF (15.24 mL) was added a solution of 1N HCl (1.524 mL, 1.524 mmol). After about 48 h additional 1N HCl (2.286 mL, 2.286 mmol) was added and the reaction mixture was heated to about 70° C. for about 2 h. After cooling to ambient temperature a solution of 1N NaOH (3.81 mL, 3.81 mmol) was added and the reaction mixture was concentrated in vacuo. The resulting solid was washed with water and dried in vacuo to provide (S)-3-(4-(3-(3-chloro-4-isopropoxyphenyl)-1,2,4-oxadiazol-5-yl)phenoxy)propane-1,2-diol (0.294 g, 94%) as a colorless solid. LC/MS (Table 1, Method c) Rₜ=2.73 min, m/z 405 (M+H)⁺.

### Example #10

### Preparation of 4-(3-(3-chloro-4-isopropoxyphenyl)-1,2,4-oxadiazol-5-yl)benzenesulfonamide

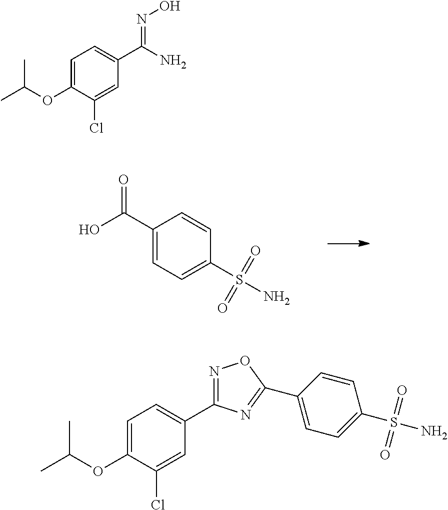

To a slurry of 4-sulfamoylbenzoic acid (1.452 g, 7.22 mmol) in DMF (8.20 mL) was added

EDC (1.383 g, 7.22 mmol) followed by HOBT hydrate (0.975 g, 7.22 mmol). After about 30 min a solution of (Z)-3-chloro-N′-hydroxy-4-isopropoxybenzimidamide in DMF (8.20 mL) was added. The reaction mixture was heated to about 140° C. for about 2 h. After cooling to RT the reaction mixture was concentrated in vacuo and purified by purified by chromatography on silica gel (eluting with EtOAc/Hep) to provide 4-(3-(3-chloro-4-isopropoxyphenyl)-1,2,4-oxadiazol-5-yl)benzenesulfonamide (1.28 g, 50%) as a colorless solid. LC/MS (Table 1, Method c) Rₜ=2.74 min, m/z 392 (M−H)⁻.

### Preparation #12a

### Preparation of tert-butyl 3,3′-(4-(3-(3-chloro-4-isopropoxyphenyl)-1,2,4-oxadiazol-5-yl)phenylsulfonylazanediyl)dipropanoate and tert-butyl 3-(4-(3-(3-chloro-4-isopropoxyphenyl)-1,2,4-oxadiazol-5-yl)phenylsulfonamido)propanoate

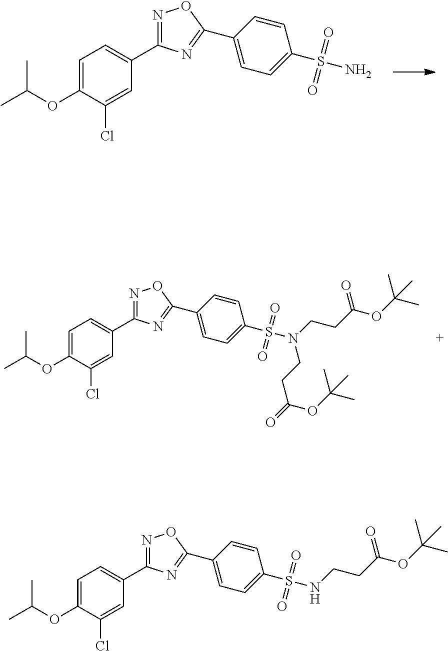

To a solution of 4-(3-(3-chloro-4-isopropoxyphenyl)-1,2,4-oxadiazol-5-yl)benzenesulfonamide (0.500 g, 1.270 mmol) in DMF (3.17 mL) was added NaH (0.056 g, 1.396 mmol). After about 10 min. tert-butyl 3-bromopropanoate (0.233 mL, 1.396 mmol) was added and the reaction mixture was heated to about 60° C. After about 48 h the reaction mixture was cooled to RT and purified by chromatography on silica gel (eluting with EtOAc/Hep) to provide tert-butyl 3,3′-(4-(3-(3-chloro-4-isopropoxyphenyl)-1,2,4-oxadiazol-5-yl)phenylsulfonylazanediyl)dipropanoate (0.24 g, 29%) as a colorless solid. LC/MS (Table 1, Method c) Rₜ=3.43 min, m/z 667 (M+NH₄)⁺, in addition to tert-butyl 3-(4-(3-(3-chloro-4-isopropoxyphenyl)-1,2,4-oxadiazol-5-yl)phenylsulfonamido)propanoate (0.28 g, 42%) as a colorless solid. LC/MS (Table 1, Method c) Rₜ=3.13 min, m/z 521 (M−H)⁻.

### Example #13

### Preparation of 3-(4-(3-(3-chloro-4-isopropoxyphenyl)-1,2,4-oxadiazol-5-yl)phenylsulfonamido)propanoic acid

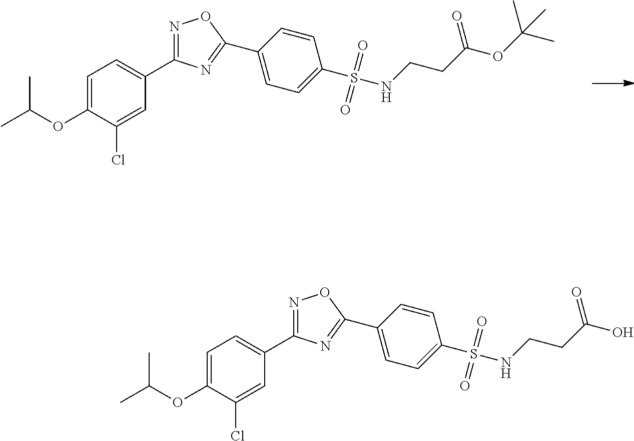

To a solution of tert-butyl 3-(4-(3-(3-chloro-4-isopropoxyphenyl)-1,2,4-oxadiazol-5-yl)phenylsulfonamido)propanoate (0.28 g, 0.536 mmol) in dichloromethane (6.0 mL) was added TFA (2.0 mL, 26.0 mmol). After about 3 h the reaction mixture was concentrated in vacuo and the resulting solid was triturated with ether, filtered and dried to provide 3-(4-(3-(3-chloro-4-isopropoxyphenyl)-1,2,4-oxadiazol-5-yl)phenylsulfonamido)propanoic acid (0.176 g, 70%) as a colorless solid. LC/MS (Table 1, Method c) Rₜ=2.54 min, m/z 466 (M+H)⁺.

### Example #14

### Preparation of 2,2′-(4-(3-(3-chloro-4-isopropoxyphenyl)-1,2,4-oxadiazol-5-yl)phenylsulfonylazanediyl)diacetic acid

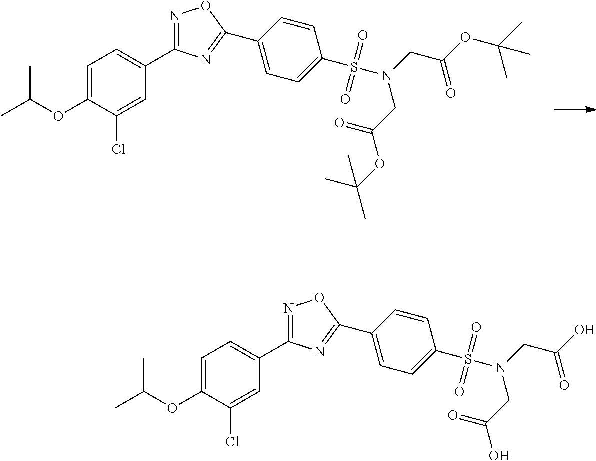

TFA (1.0 mL, 12.98 mmol) was added to a stirred mixture of tert-butyl 2,2′-(4-(3-(3-chloro-4-isopropoxyphenyl)-1,2,4-oxadiazol-5-yl)phenylsulfonylazanediyl)diacetate (0.106 g, 0.170 mmol), dichlorom and then concentrated in vacuo. The resulting residue was triturated with diethyl ether, filtered and dried to give 2,2′-(4-(3-(3-chloro-4-isopropoxyphenyl)-1,2,4-oxadiazol-5-yl)phenylsulfonylazanediyl)diacetic acid (63 mg, 0.122 mmol) as a white solid.

LC/MS (Table 1, Method c) Rₜ=1.84 min, m/z 508.38 (M−H)⁻.

### Preparation #12b

### Preparation of tert-butyl 2,2′-(4-(3-(3-chloro-4-isopropoxyphenyl)-1,2,4-oxadiazol-5-yl)phenylsulfonylazanediyl)diacetate and tert-butyl 2-(4-(3-(3-chloro-4-isopropoxyphenyl)-1,2,4-oxadiazol-5-yl)phenylsulfonamido)acetate

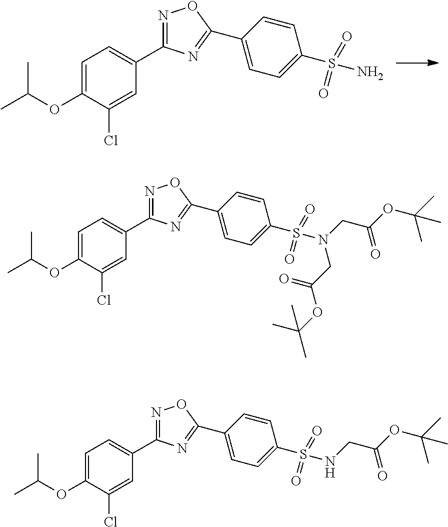

Powdered K₂CO₃ (0.190 g, 1.374 mmol) was added dropwise to a stirred mixture of 4-(3-(3-chloro-4-isopropoxyphenyl)-1,2,4-oxadiazol-5-yl)benzenesulfonamide (0.492 g, 1.249 mmol) in anhydrous ACN (6.25 mL) under N₂. Tert-butyl 2-bromoacetate (0.203 mL 1.374 mmol) was then added and the mixture heated to about 80° C. for about 3 h. The reaction mixture (suspension) was concentrated in vacuo and the resulting material triturated in DCM and filtered. The filtrate was concentrated and purified directly via Analogix® system using RediSep™ RS 40 g column, with a gradient of 0-40% EtOAc/Heptane over 40 min. at 30 mL/min. Fractions containing product were combined and concentrated. This gave tert-butyl 2,2′-(4-(3-(3-chloro-4-isopropoxyphenyl)-1,2,4-oxadiazol-5-yl)phenylsulfonylazanediyl)diacetate (249 mg, 0.400 mmol) as a sticky white solid LC/MS (Table 1, Method c) Rₜ=3.17 min, m/z 639 (M+NH₄)⁺ and tert-butyl 2-(4-(3-(3-chloro-4-isopropoxyphenyl)-1,2,4-oxadiazol-5-yl)phenylsulfonamido)acetate (121 mg, 0.238 mmol) as a white solid LC/MS (Table 1, Method c) Rₜ=2.81 min, m/z 508 (M+H)⁺.

### Example #16

### Preparation of 2-(4-(3-(3-chloro-4-isopropoxyphenyl)-1,2,4-oxadiazol-5-yl)phenylsulfonamido)acetic acid

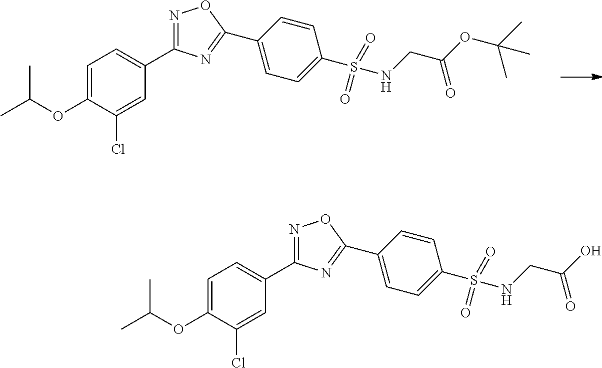

TFA (2.0 mL, 26.0 mmol) was added dropwise to a stirred mixture of tert-butyl 2-(4-(3-(3-chloro-4-isopropoxyphenyl)-1,2,4-oxadiazol-5-yl)phenylsulfonamido)acetate (0.121 g, 0.238 mmol), DCM (5.0 mL) under N₂. The mixture was stirred at ambient temperature for about 3 h then concentrated in vacuo. The resulting solid was triturated in ether, filtered and dried to yield 2-(4-(3-(3-chloro-4-isopropoxyphenyl)-1,2,4-oxadiazol-5-yl)phenylsulfonamido)acetic acid (46 mg, 0.102 mmol) as a white solid. LC/MS (Table 1, Method c) Rₜ=2.14 min, m/z 450.34 (M−H)⁻

### Preparation #12c

### Preparation of tert-butyl 2-(5-(3-(3-chloro-4-isopropoxyphenyl)-1,2,4-oxadiazol-5-yl)-3,4-dihydroisoquinolin-2(1H)-yl)acetate

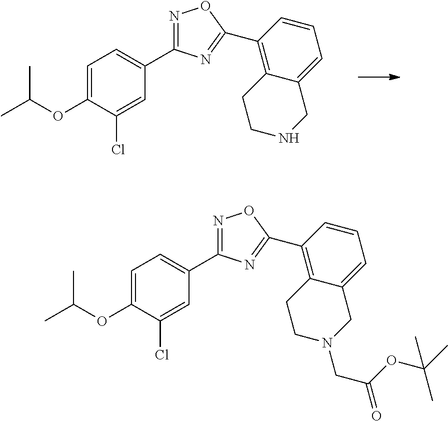

To a solution of 3-(3-chloro-4-isopropoxyphenyl)-5-(1,2,3,4-tetrahydroisoquinolin-5-yl)-1,2,4-oxadiazole (0.0726 g, 0.196 mmol) in DMF (1.963 mL) was added K₂CO₃ (0.054 g, 0.393 mmol) followed by tert-butyl bromoacetate (0.030 mL, 0.206 mmol). After about 48 h the reaction mixture was filtered, concentrated in vacuo and purified by chromatography to provide tert-butyl 2-(5-(3-(3-chloro-4-isopropoxyphenyl)-1,2,4-oxadiazol-5-yl)-3,4-dihydroisoquinolin-2(1H)-yl)acetate as a colorless oil that solidified on standing. LC/MS (Table 1, Method c) Rₜ=3.41 min, m/z 486 (M+H)⁺.

### Preparation #12d

### Preparation of tert-butyl 5-(3-(3-chloro-4-isopropoxyphenyl)-1,2,4-oxadiazol-5-yl)-3,4-dihydroisoquinoline-2(1H)-carboxylate

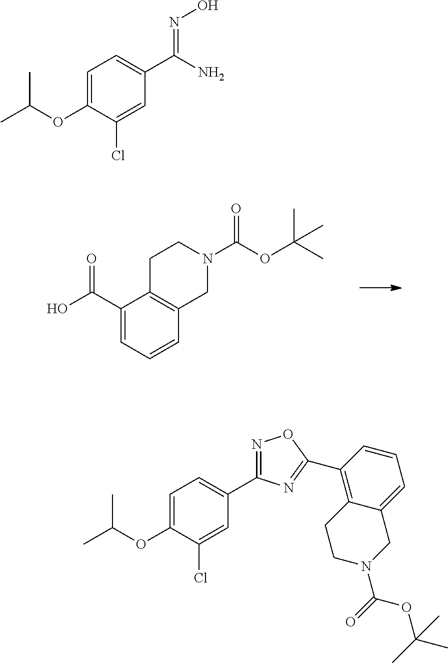

To a slurry of 2-(tert-butoxycarbonyl)-1,2,3,4-tetrahydroisoquinoline-5-carboxylic acid (0.380 g, 1.371 mmol) in DMF (1.662 mL) was added EDC (0.263 g, 1.371 mmol) followed by HOBT hydrate. After about 1 h a solution of (Z)-3-chloro-N′-hydroxy-4-isopropoxybenzimidamide (0.285 g, 1.246 mmol) in DMF (0.831 mL) was added and the reaction mixture was heated to about 140° C. for about 1 h. The reaction mixture was concentrated in vacuo and purified by chromatography on silica gel to provide tert-butyl 5-(3-(3-chloro-4-isopropoxyphenyl)-1,2,4-oxadiazol-5-yl)-3,4-dihydroisoquinoline-2(1H)-carboxylate (0.403 g, 69%) as a colorless oil. LC/MS (Table 1, Method c) Rₜ=3.43 min, m/z 471 (M+H)⁺.

### Example #19

### Preparation of 3-(3-chloro-4-isopropoxyphenyl)-5-(1,2,3,4-tetrahydroisoquinolin-5-yl)-1,2,4-oxadiazole

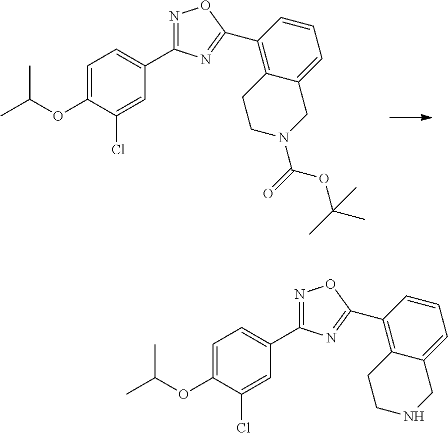

To a solution of tert-butyl 5-(3-(3-chloro-4-isopropoxyphenyl)-1,2,4-oxadiazol-5-yl)-3,4-dihydroisoquinoline-2(1H)-carboxylate (0.403 g, 0.858 mmol) in dioxane (17.15 mL) was added a 4N solution of HCl in 1,4-dioxane (3.86 mL, 15.44 mmol). After about 15 h the reaction mixture was filtered. The resulting solid was partitioned between EtOAc and saturated NaHCO₃. The organic layer was separated, dried (MgSO₄) filtered and concentrated in vacuo to provide 3-(3-chloro-4-isopropoxyphenyl)-5-(1,2,3,4-tetrahydroisoquinolin-5-yl)-1,2,4-oxadiazole (0.230 g, 73%) as a colorless solid. LC/MS (Table 1, Method c) Rₜ=2.00 min, m/z 372 (M+H)⁺.

### Example #20

### Preparation of 2-(5-(3-(3-chloro-4-isopropoxyphenyl)-1,2,4-oxadiazol-5-yl)-3,4-dihydroisoquinolin-2(1H)-yl)acetic acid

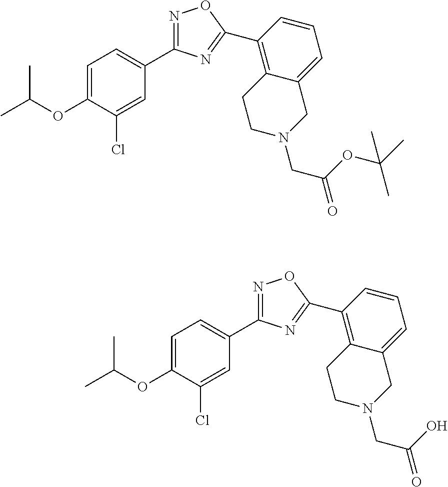

To a solution of tert-butyl 2-(5-(3-(3-chloro-4-isopropoxyphenyl)-1,2,4-oxadiazol-5-yl)-3,4-dihydroisoquinolin-2(1H)-yl)acetate (0.1319 g, 0.273 mmol) in dichloromethane (10 mL was added triisopropylsilane (0.056 mL, 0.273 mmol) followed by TFA (2 mL). After about 15 h reaction mixture was concentrated in vacuo. The resulting solid was triturated in ether, filtered and dried to provide 2-(5-(3-(3-chloro-4-isopropoxyphenyl)-1,2,4-oxadiazol-5-yl)-3,4-dihydroisoquinolin-2(1H)-yl)acetic acid (0.138 g, 93%) as an off-white solid. LC/MS (Table 1, Method c) Rₜ=2.00 min, m/z 428 (M+H)⁺.

### Preparation #12e

### Preparation of tert-butyl 3-(5-(3-(3-chloro-4-isopropoxyphenyl)-1,2,4-oxadiazol-5-yl)-3,4-dihydroisoquinolin-2(1H)-yl)propanoate

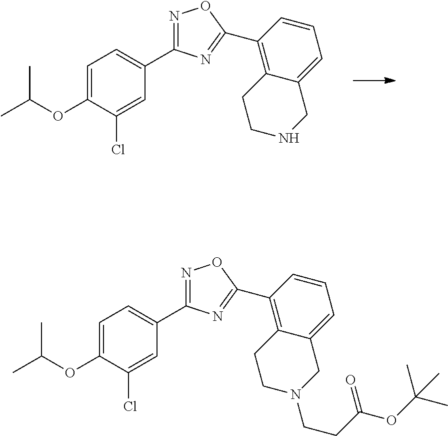

To 3-(3-chloro-4-isopropoxyphenyl)-5-(1,2,3,4-tetrahydroisoquinolin-5-yl)-1,2,4-oxadiazole (0.1088 g, 0.294 mmol) in DMF (2.94 mL) (briefly heated to about 40° C. for complete dissolution) was added K₂CO₃ (0.081 g, 0.588 mmol) and tert-butyl 3-bromopropanoate (0.046 mL, 0.276 mmol) and the mixture stirred at ambient temperature for about 2 h. Additional tert-butyl 3-bromopropanoate (0.053 mL, 0.315 mmol) was added and the reaction was stirred at about 60° C. over the weekend. Additional tert-butyl 3-bromopropanoate (0.053 mL, 0.315 mmol) was added and the reaction continued heating at about 60° C. overnight. Additional K₂CO₃ (0.041 g, 0.294 mmol) was added, followed by tert-butyl 3-bromopropanoate (0.053 mL, 0.315 mmol). The reaction was heated at about 60° C. overnight. The reaction mixture was filtered and the filtrate concentrated in vacuo to give ˜179 mg of crude yellow oil. The crude residue was purified via Analogix® system using RediSep™ RS 12 g column, with a gradient of 0-45% EtOAc/Heptane over 35 min. at 15 mL/min. Fractions 23-28 were combined and concentrated to yield tert-butyl 3-(5-(3-(3-chloro-4-isopropoxyphenyl)-1,2,4-oxadiazol-5-yl)-3,4-dihydroisoquinolin-2(1H)-yl)propanoate (91 mg, 0.183 mmol) as light yellow oil. LC/MS (Table 1, Method c) Rₜ=3.39 min, m/z 500.72 (M+H)⁺.

### Example #22

### Preparation of 3-(5-(3-(3-chloro-4-isopropoxyphenyl)-1,2,4-oxadiazol-5-yl)-3,4-dihydroisoquinolin-2(1H)-yl)propanoic acid, TFA salt

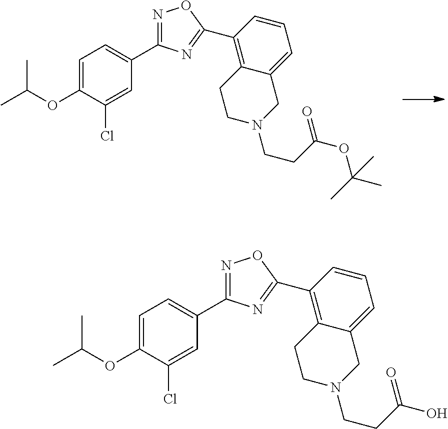

To tert-butyl 3-(5-(3-(3-chloro-4-isopropoxyphenyl)-1,2,4-oxadiazol-5-yl)-3,4-dihydroisoquinolin-2 (1H)-yl)propanoate (0.091 g, 0.183 mmol) in dichloromethane (6.0 mL) TFA (1.5 mL) was added and the mixture stirred at ambient temperature overnight. The reaction mixture was concentrated in vacuo and the resulting crude product was dissolved in small amount of DCM. Ether was added until a solid precipitated out. The mixture was filtered, rinsed with ether and dried to give 3-(5-(3-(3-chloro-4-isopropoxyphenyl)-1,2,4-oxadiazol-5-yl)-3,4-dihydroisoquinolin-2(1H)-yl)propanoic acid, TFA salt (74.7 mg, 0.134 mmol) as light yellow solid. LC/MS (Table 1, Method c) Rₜ=2.04 min, m/z 442.25 (M+H)⁺. 1H NMR (400 MHz, DMSO) δ ppm 8.18-8.11 (dd, J=2.07 6.76 Hz, 1H), 8.11-8.06 (d, J=2.01 Hz, 1H), 8.06-7.99 (J=2.02, 8.64 Hz, 1H), 7.61-7.53 (J=6.58, 6.58 Hz, 1H), 7.45-7.37 (J=8.8 Hz, 1H), 4.90-4.78 (m, 1H), 4.65-4.46 (s, 2H), 3.71-3.51 (s, 3H), 3.51-3.38 (J=6.87, 6.87 Hz, 3H), 2.91-2.81 (t, J=7.32, 7.32 Hz, 2H) and 1.39-1.33 (d, 6H)

### Preparation #12

### 4-isopropoxy-3-(trifluoromethyl)benzonitrile

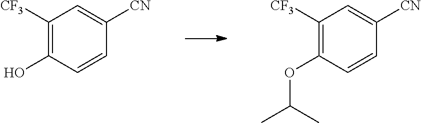

Under an atmosphere of nitrogen a mixture of 4-hydroxy-3-(trifluoromethyl)benzonitrile (5.89 g, 31.5 mmol) and triphenylphosphine (13.21 g, 50.4 mmol) in anhydrous THF (200 mL) was stirred for about 5 min at ambient temperature. To the solution DBAD (11.60 g, 50.4 mmol) was added, stirred about 5 min before the addition of 2-propanol (3.03 mL, 39.3 mmol). The mixture was stirred at ambient temperature for about 72 h. The solvent was removed under reduced pressure. The resulting oil was triturated with 30-60° C. pet/ether (200 mL), filtered to remove phosphine oxide and the crude product was purified further by elution through silica with heptane/ethyl acetate (4:1). The isolated oil was dissolved in dichloromethane (200 mL) and stirred with TFA (4.85 mL, 63.0 mmol) for about 90 min at ambient temperature. The solution was basified with 2.5 N NaOH (30 mL) and the product was partitioned between DCM and the basic aqueous phase to give the crude 4-isopropoxy-3-(trifluoromethyl)benzonitrile (6.56, 91%). LC/MS (Table 1, Method a) Rₜ=2.32 min, 1H NMR (400 MHz, CDCl3) 7.85 (d, 1H), 7.75 (dd, 1H), 7.06 (d, 1H), 4.73 (m, 1H), 1.41 (dd, 6H).

### Preparation #13

### (Z)—N′-hydroxy-4-isopropoxy-3-(trifluoromethyl)benzimidamide

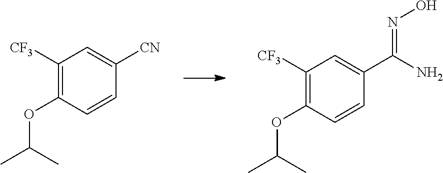

Under an atmosphere of nitrogen, 4-isopropoxy-3-(trifluoromethyl)benzonitrile (6.5 g, 28.4 mmol) and 50% aqueous hydroxylamine (5.21 mL, 85 mmol) in EtOH (20.0 mL) was heated at about 60° C. for about 18 h. Solvents were removed in vacuo and the residue was azeotroped with MeOH. The residual solid was purified by precipitation from an ethyl acetate/30-60° C. pet/ether mixture (1:2) to give (Z)—N′-hydroxy-4-isopropoxy-3-(trifluoromethyl)benzimidamide (2.51 g, 33.8%) LC/MS (Table 1, Method b) Rₜ=1.89 min, m/z 263.13 (M+H)⁺.

### Preparation #14

### (S)-3-chloro-4-(tetrahydrofuran-3-yloxy)benzonitrile

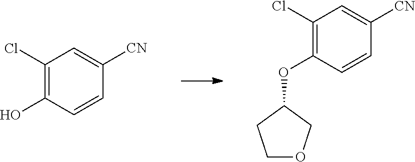

Under an atmosphere of nitrogen, a mixture of 3-chloro-4-hydroxybenzonitrile (8.70 g, 56.7 mmol) and triphenylphosphine (23.77 g, 91 mmol) in anhydrous THF (218 mL) was stirred for about 5 min at ambient temperature. To the solution DBAD (20.87 g, 91 mmol) was added, stirred about 5 min before the addition of (S)-(+)-3-hydroxytetrahydrofuran (3.87 mL, 56.7 mmol) in THF (10 mL). The mixture was stirred at ambient temperature for about 24 h. The solvent was removed under reduced pressure. The residue was dissolved in dichloromethane (200 mL) and stirred with TFA (21.82 mL, 283 mmol) for about 90 min. at ambient temperature. The solution was basified with aqueous sodium hydroxide and the product was partitioned between DCM and the basic aqueous phase. The DCM was dried over magnesium sulphate, filtered and solvent removed under reduced pressure to give an oil. The oil was stirred with hot 30-60° C. pet/ether (200 mL), cooled and filtered. The solvent was removed under reduced pressure to give the crude (S)-3-chloro-4-(tetrahydrofuran-3-yloxy)benzonitrile (11.2 g). Rₜ 2.06 min, m/z 378.2 (M+H)⁺.

### Preparation #15

### (S,Z)-3-chloro-N′-hydroxy-4-(tetrahydrofuran-3-yloxy)benzimidamide

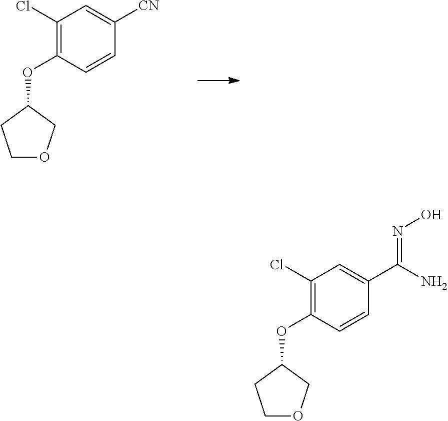

Under an atmosphere of nitrogen, (S)-3-chloro-4-(tetrahydrofuran-3-yloxy)benzonitrile (11.2 g, 50.1 mmol) and 50% aqueous hydroxylamine (3.31 g, 50.1 mmol) in EtOH (150.0 mL) was heated at about 60° C. for about 18 h. Solvents were removed in vacuo and the residue was azeotroped with MeOH. The residual solid was purified by precipitation from an ethyl acetate/30-60° C. pet/ether mixture (1:2) to give (S,Z)-3-chloro-N′-hydroxy-4-(tetrahydrofuran-3-yloxy)benzimidamide (5.3 g) LC/MS (Table 1, Method b) Rₜ=1.52 min, m/z 257.09 (M+H)⁺.

### Preparation #16

### 4-morpholino-3-(trifluoromethyl)benzonitrile

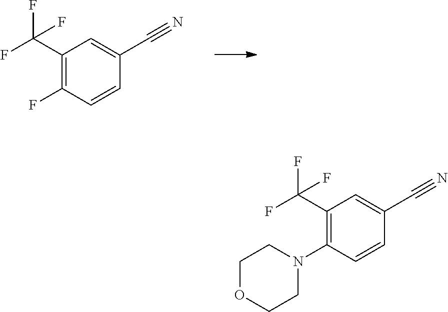

To a solution of 4-fluoro-3-(trifluoromethyl)benzonitrile (15 g, 79 mmol) in dimethylsulfoxide (160 mL) was added morpholine (13.8 mL, 159 mmol) and potassium carbonate (16.4 g, 119 mmol). The mixture was heated at about 90° C. for about 18 h. The mixture was cooled to ambient temperature and the solid was removed by filtration. The filtrate was partitioned between ethyl acetate (1.8 L) and water (1.5 L). The organic layer was washed with water (1.0 L) and brine (1.0 L) and dried over anhydrous magnesium sulphate. The solvent was removed in vacuo to give 4-morpholino-3-(trifluoromethyl)benzonitrile (17.25 g, 85%). ¹H NMR (DMSO-d6, 400 MHz) δ ppm 8.18 (d, J=2.05 Hz, 1H), 8.09 (dd, J=8.51, 2.06 Hz, 1H), 7.60 (d, J=8.52 Hz, 1H), 3.69-3.75 (m, 4H), 2.97-3.04 (m, 4H).

### Preparation #17

### N′-hydroxy-4-morpholino-3-(trifluoromethyl)benzimidamide

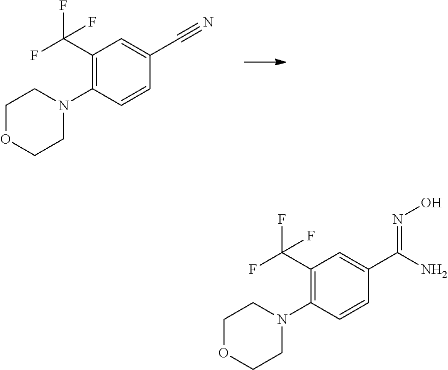

To a solution of 4-morpholino-3-(trifluoromethyl)benzonitrile (17.3 g, 67.3 mmol) in ethanol (400 mL) was added a 50% aqueous solution of hydroxylamine (4.9 mL, 74.1 mmol) dropwise. The mixture was heated at about 65° C. for about 24 h. The mixture was cooled to ambient temperature and the solid was removed by filtration. The filtrate was partitioned between ethyl acetate (1.8 L) and water (1.5 L). The organic layer was washed with water (1.0 L) and brine (1.0 L) and dried over anhydrous magnesium sulphate. The solvent was removed in vacuo to give N′-hydroxy-4-morpholino-3-(trifluoromethyl)benzimidamide (18.6 g, 91%) as a mixture of syn/anti isomers. LC/MS (Table 1, Method b) Rₜ=1.85 min, m/z 290.15 (M+H)⁺; ¹H NMR (DMSO-d6, 400 MHz) δ ppm 9.75 (s, 1H), 8.09-8.16 (m, 1H), 7.89-7.96 (m, 1H), 7.52-7.58 (m, 1H), 3.66-3.72 (m, 4H), 2.83-2.93 (m, 4H).

### Preparation #18

### 5-Methoxy-3,6-dihydro-2H-pyrazine-1-carboxylic acid benzyl ester

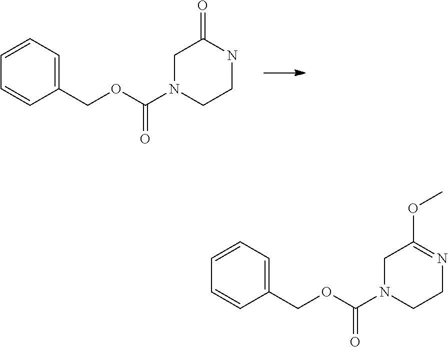

A solution of benzyl 3-oxopiperazine-1-carboxylate (2.50 g, 10.67 mmol) in CH₂Cl₂ (100 mL) was cooled to about 0° C. and treated with Na₂CO₃ (23.0 g, 217 mmol) for about 10 min. Neat trimethyloxonium tetrafluoroborate (5.50 g, 37.2 mmol) was added in one portion, then the reaction is allowed to warm to RT for about 6 h. The reaction was poured into water (100 mL), and the layers were separated. The aqueous layer was re-extracted with 50 mL CH₂Cl₂ and the combined organic layers were washed with brine (100 mL). The organic layer was dried over sodium sulfate, filtered and concentrated to yield 5-methoxy-3,6-dihydro-2H-pyrazine-1-carboxylic acid benzyl ester (2.51 g, 95%) as an oil. LC/MS (Table 1, Method a) Rₜ=3.00 min, m/z 249.24 (M+H)+⁻; ¹H NMR (400 MHz, DMSO-d6) δ ppm 7.36 (m, 5H), 5.16 (s, 2H), 3.96 (s, 2H), 3.68 (s, 3H), 3.54 (s, 2H), 3.47 (m, 2H)

### Preparation #19

### 3-Methyl-5,6-dihydro-8H-imidazo[1,2-a]pyrazine-7-carboxylic acid benzyl ester

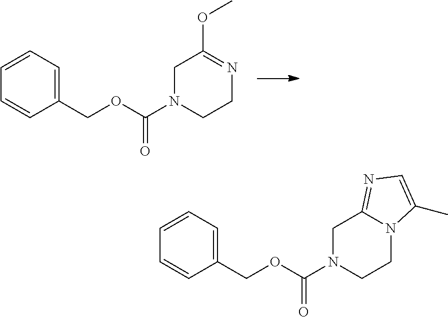

To a solution of 3-methoxy-5,6-dihydropyrazine-1(2H)-carboxylate (4.48 g, 18.03 mmol) in MeOH (200 mL) was added propargylamine (6.18 mL, 90 mmol) at RT. The mixture was heated at reflux for about 5 h, then cooled to RT and concentrated. The residue was dissolved in 1N HCl (100 mL) and washed with 3×75 mL ethyl acetate. The aqueous solution was neutralized with solid Na₂CO₃ and extracted with 2×100 mL ethyl acetate. The combined extracts were washed with 100 mL saturated NaCl solution, filtered and concentrated. The residue was triturate with ether, filtered and dried under reduced pressure to yield 3-Methyl-5,6-dihydro-8H-imidazo[1,2-a]pyrazine-7-carboxylic acid benzyl ester (2.91 g, 60%) as an off-white solid. LC/MS (Table 1, Method a) Rₜ=3.07 min, m/z 272.11 (M+H)+⁻; ¹H NMR (400 MHz, DMSO-d6) δ ppm 7.30 (m, 5H), 6.58 (q, 1H), 5.13 (s, 2H), 4.55 (s, broad, 2H), 3.84 (s, 4H), 2.10 (s, 3H).

### Preparation #20

### 2-Iodo-3-methyl-5,6-dihydro-8H-imidazo[1,2-a]pyrazine-7-carboxylic acid benzyl ester

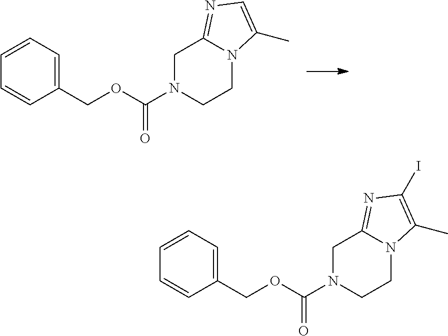

To a solution of benzyl 3-methyl-5,6-dihydroimidazo[1,2-a]pyrazine-7(8H)-carboxylate (1.085 g, 4.00 mmol) in 1,2-dichloroethane (60 mL) was added NIS (4.50 g, 20.00 mmol) and the reaction was heated at reflux for about 1 h. The reaction was cooled to RT and poured into 100 mL of saturated 5% sodium thiosulfate solution. The layers were separated and the aqueous layer was re-extracted with 1,2-dichloroethane (40 mL). The combined organic layers were washed with water (100 mL), dried over sodium sulfate, filtered and concentrated. Product was extracted from the residue by trituration with 3×50 mL portions of ether. The extract was filtered and concentrated to yield 2-Iodo-3-methyl-5,6-dihydro-8H-imidazo[1,2-a]pyrazine-7-carboxylic acid benzyl ester (1.42 g, 89%) as a pale yellow oil. LC/MS (Table 1, Method a) Rₜ=3.32 min, m/z 398.59 (M+H)+⁻; ¹H NMR (400 MHz, CHCl₃) δ ppm 7.35 (m, 5H), 5.13 (s, 2H), 4.56 (s, broad, 2H), 4.38 (t, 2H), 3.82 (s, broad, 2H), 2.09 (s, 3H)

### Preparation #21

### 3-Methyl-5,6-dihydro-8H-imidazo[1,2-a]pyrazine-2,7-dicarboxylic acid 7-benzyl ester

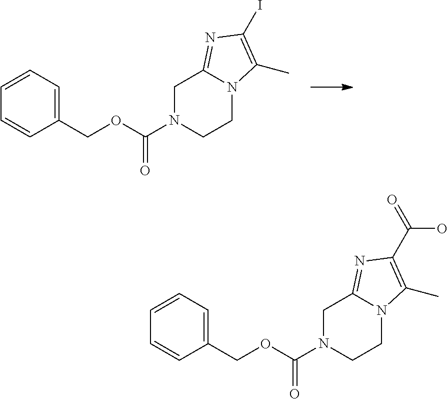

A solution of benzyl 2-iodo-3-methyl-5,6-dihydroimidazo[1,2-a]pyrazine-7(8H)-carboxylate (900 mg, 2.266 mmol) in dry THF (25 mL) was cooled to about 0° C. and ethyl magnesiumbromide (1.888 mL, 5.66 mmol) was added at such a rate as to maintain the reaction temperature below about 2.5° C. The reaction was stirred under nitrogen at about 0° C. for about 15 min. then the reaction was quenched with a stream of carbon dioxide. The reaction was concentrated to solids and acetic acid (0.60 mL, 10.48 mmol) ethyl acetate (50 mL) were added and the suspension was stirred vigorously at RT for about 15 min. The resulting solid was filtered and washed with an additional 15 mL ethyl acetate. The residue was dissolved in 10 mL water plus 2 N HCl to pH 4, then washed 2 times with 10 mL ether then extracted with 4×20 mL CH₂Cl₂. The combined organic extracts were dried over sodium sulfate, filtered and evaporated under reduced pressure to yield 3-Methyl-5,6-dihydro-8H-imidazo[1,2-a]pyrazine-2,7-dicarboxylic acid 7-benzyl ester (374 mg, 52%) as a foam.

LC/MS (Table 1, Method a) Rₜ=2.28 min, m/z 316.10 (M+H)+⁻; 1H NMR (400 MHz, DMSO-d6) δ ppm 7.35 (m, 5H), 5.11 (s, 2H), 4.56 (s, broad, 2H), 3.88 (m, 2H), 3.83 (s, broad, 2H), 2.36 (s, 3H).

### Preparation #22

### Preparation of 2-[3-(3-Chloro-4-isopropoxy-phenyl)-[1,2,4]oxadiazol-5-yl]-3-methyl-5,6-dihydro-8H-imidazo[1,2-a]pyrazine-7-carboxylic acid benzyl ester

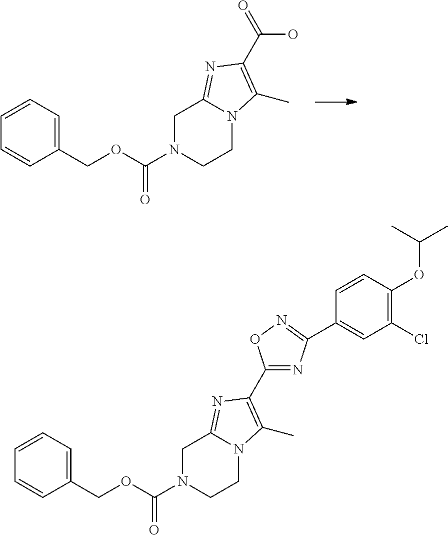

To a solution of 7-(benzyloxycarbonyl)-3-methyl-5,6,7,8-tetrahydroimidazo[1,2-a]pyrazine-2-carboxylic acid (370 mg, 1.173 mmol) in DCM (10 mL) was added oxalyl chloride (2.054 mL, 23.47 mmol) and DMF (5 μL). The reaction was stirred for about 1 h and concentrated. A solution of (E)-3-chloro-N′-hydroxy-4-isopropoxybenzimidamide (268 mg, 1.173 mmol) in pyridine (10.00 mL) was added and the reaction was stirred at RT for about 30 min. The reaction was treated with acetyl chloride (0.092 mL, 1.291 mmol) and then was heated at 115° C. under nitrogen for about 4 h. The reaction was cooled, concentrated and partitioned between saturated Na₂CO₃ and methylene chloride. The organic layer was washed with water, dried (sodium sulfate), filtered and concentrated under reduced pressure. The residue was purified on silica gel using 80:20/methylene chloride:ethyl acetate as the eluent to yield 2-[3-(3-chloro-4-isopropoxy-phenyl)-[1,2,4]oxadiazol-5-yl]-3-methyl-5,6-dihydro-8H-imidazo[1,2-a]pyrazine-7-carboxylic acid benzyl ester (173 mg, 29%) as an off-white solid. LC/MS (Table 1, Method a) Rₜ=4.34 min, m/z 508.24 (M+H)+⁻; ¹H NMR (400 MHz, DMSO-d6) δ ppm 7.98 (d, 1H), 7.93 (d, d, 1H), 7.35 (m, 6H), 5.12 (s, 2H), 4.78 (m, 1H), 4.66 (s, broad, 2H), 3.99 (m, 2H), 3.88 (s, broad, 2H), 2.57 (s, 3H), 1.31 (d, 6H).

### Example #23

### Preparation of 2-[3-(3-Chloro-4-isopropoxy-phenyl)-[1,2,4]oxadiazol-5-yl]-3-methyl-5,6,7,8-tetrahydro-imidazo[1,2-a]pyrazine

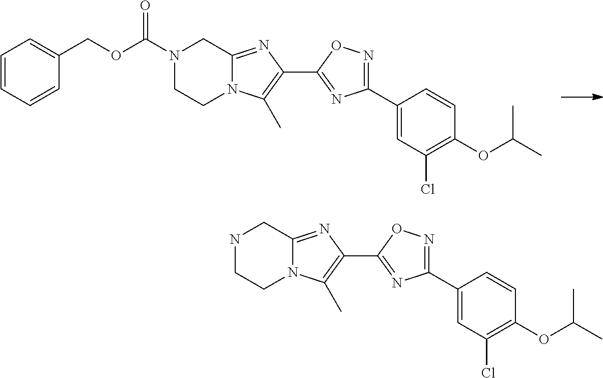

A solution of benzyl 2-(3-(3-chloro-4-isopropoxyphenyl)-1,2,4-oxadiazol-5-yl)-3-methyl-5,6-dihydroimidazo[1,2-a]pyrazine-7(811)-carboxylate (160 mg, 0.315 mmol) in 33% HBr solution in acetic acid (2.00 mL) containing triisopropylsilane (0.065 mL, 0.315 mmol)) was stirred at RT under nitrogen for about 10 min. Ether (20 mL) was added to precipitate the product. The resulting solid was filtered off, treated with saturated bicarbonate solution (10 mL) and extracted with methylene chloride (2×10 mL). The combined organic layers were dried over sodium sulfate, filtered, concentrated to solids and dried under reduced pressure to yield 2-[3-(3-chloro-4-isopropoxy-phenyl)-[1,2,4]oxadiazol-5-yl]-3-methyl-5,6,7,8-tetrahydro-imidazo[1,2-a]pyrazine (113 mg, 96%) as an off-white solid. LC/MS (Table 1, Method a) Rₜ=3.14 min, m/z 374.24 (M+H)+⁻; ¹H NMR (400 MHz, DMSO-d6) δ ppm 8.01 (d, 1H), 7.97 (d, d, 1H), 7.36 (d, 1H), 4.81 (m, 1H), 4.66 (s, 2H), 3.90 (s, 2H), 3.87 (t, 2H), 3.12 (t, 2H), 2.60 (s, 3H), 1.34 (d, 6H).

### Example #24

### Preparation of 1-{2-[3-(3-Chloro-4-isopropoxy-phenyl)-[1,2,4]oxadiazol-5-yl]-3-methyl-5,6-dihydro-8H-imidazo[1,2-a]pyrazin-7-yl}-ethanone

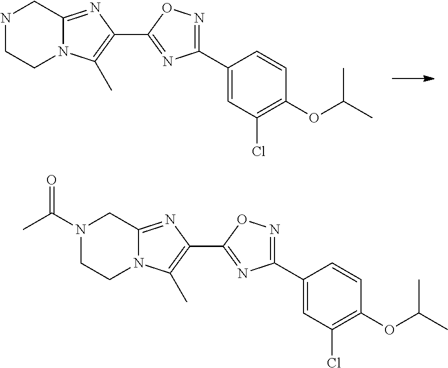

To a solution of 2-(3-chloro-4-isopropoxyphenyl)-3-methyl-5,6,7,8-tetrahydroimidazo[1,2-a]pyrazine (32 mg, 0.105 mmol) in methylene chloride (2.0 mL) was added acetyl chloride (7.50 μl, 0.105 mmol) at RT. The mixture was stirred at RT for 4 h and concentrated. The residue was purified by reverse phase HPLC to yield 1-{2-[3-(3-chloro-4-isopropoxy-phenyl)-[1,2,4]oxadiazol-5-yl]-3-methyl-5,6-dihydro-8H-imidazo[1,2-a]pyrazin-7-yl}-ethanone (31 mg, 86%) as an off-white solid. LC/MS (Table 1, Method a) Rₜ=3.46 min, m/z 416.20 (M+H)⁺; ¹H NMR (400 MHz, DMSO-d6) δ ppm 8.02 (d, 1H), 8.97 (d, d, 1H), 7.37 (d, 1H), 4.81 (m, 2H), 4.70 (s, 2H), 3.90 (s, 2H), 4.07 (t0m, 1H), 3.95 (m, 3H), 2.62 (s, 3H), 2.14 (m, 3H), 1.34 (d, 6H).

### Preparation #22a

### Preparation of {2-[3-(3-Chloro-4-isopropoxy-phenyl)-[1,2,4]oxadiazol-5-yl]-3-methyl-5,6-dihydro-8H-imidazo[1,2-a]pyrazin-7-yl}-acetic acid tert-butyl ester

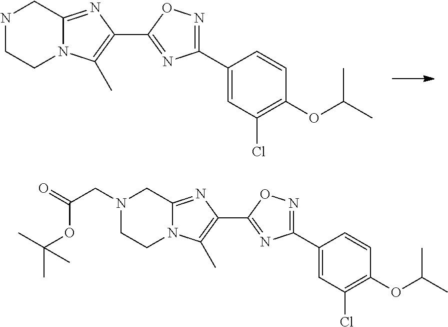

To a solution of 3-(3-chloro-4-isopropoxyphenyl)-5-(3-methyl-5,6,7,8-tetrahydroimidazo[1,2-a]pyrazin-2-yl)-1,2,4-oxadiazole (50.0 mg, 0.134 mmol) in DMF (1.0 mL) at RT was added sodium carbonate (28.4 mg, 0.267 mmol) and tert-butyl bromoacetate (0.021 mL, 0.140 mmol) at RT. The reaction was continued overnight. The reaction was filtered and concentrated. The residue was dissolved in ethyl acetate (10 mL), washed with brine (10 mL), dried over sodium sulphate, filtered and concentrated to yield {2-[3-(3-chloro-4-isopropoxy-phenyl)-[1,2,4]oxadiazol-5-yl]-3-methyl-5,6-dihydro-8H-imidazo[1,2-a]pyrazin-7-yl}-acetic acid tert-butyl ester (35 mg, 54%) as an off-white foam which was used in the next step without further purification. LC/MS (Table 1, Method a) Rₜ=4.32 min, m/z 488.29 (M+H)⁺

### Example #26

### Preparation of {2-[3-(3-Chloro-4-isopropoxy-phenyl)-[1,2,4]oxadiazol-5-yl]-3-methyl-5,6-dihydro-8H-imidazo[1,2-a]pyrazin-7-yl}-acetic acid, trifluoroacetic acid salt

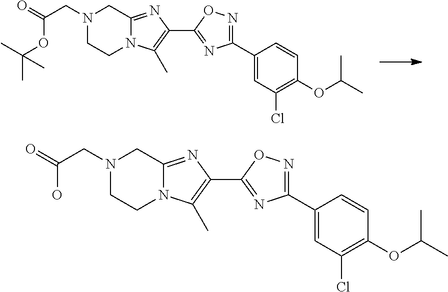

To a solution of tert-butyl 2-(2-(3-(3-chloro-4-isopropoxyphenyl)-1,2,4-oxadiazol-5-yl)-3-methyl-5,6-dihydroimidazo[1,2-a]pyrazin-7(8H)-yl)acetate (32 mg, 0.066 mmol) and triisopropylsilane (0.013 mL, 0.066 mmol) in methylene chloride (2.0 mL) was added TFA (2.0 mL) at RT for about 3 h. The reaction was diluted with ether (20 mL) and the product was filtered off and dried under reduced pressure. LC/MS (Table 1, Method a) Rₜ=2.99 min, m/z 432.23 (M+H)+⁻; ¹H NMR (400 MHz, DMSO-d6) δ 8.0 (m, 2H), 7.36 (m, 1H), 4.81 (m, 1H), 3.97 (m, 2H), 3.84 (m, 2H), 3.46 (m, 2H), 3.09 (m, 2H), 2.59 (s, 3H), 1.33 (d, 6H).

### Preparation #23

### 2-Methyl-imidazo[1,2-a]pyrazine-3-carboxylic acid ethyl ester

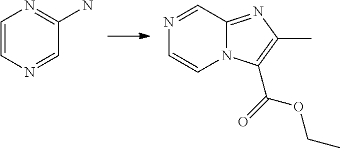

A solution of pyrazin-2-amine (3.6 g, 37.9 mmol) and ethyl 2-chloro-3-oxobutanoate (5.24 mL, 37.9 mmol) in ethanol (30 mL) was heated at reflux for about 9 h. A 1 N solution of HCl in ether was added and the mixture was concentrated under reduced pressure. The residue was triturated with 3×50 mL ACN and filtered to yield crude 2-methyl-imidazo[1,2-a]pyrazine-3-carboxylic acid ethyl ester (4.5 g, 58%) as an amorphous solid which was used in the next step without further purification.

### Preparation #24

### 2-Methyl-imidazo[1,2-a]pyrazine-3-carboxylic acid

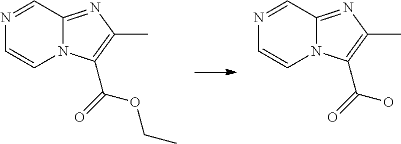

A solution of sodium hydroxide (1.754 g, 43.9 mmol) in water (25 mL) was added to crude ethyl 2-methylimidazo[1,2-a]pyrazine-3-carboxylate (4.5 g, 21.93 mmol). The reaction is exothermic and goes to completion in minutes without additional heating. The mixture was acidified with concentrated HCl to about pH 5. The solution was injected onto a preparative C18 column and washed with water and then eluted with 20% CH₃CN/water. The product fractions were combined and concentrate to yield 2-methyl-imidazo[1,2-a]pyrazine-3-carboxylic acid (250 mg, 6%) as a tan solid. LC/MS (Table 1, Method a) Rₜ=0.84 min, m/z 176.18 (M−H)⁻; ¹H NMR (400 MHz, DMSO-d6) δ 9.12 (m, 2H), 8.12 (m, 1H), 2.66 (s, 3H).

### Example #27

### Preparation of 3-[3-(3-Chloro-4-isopropoxy-phenyl)-[1,2,4]oxadiazol-5-yl]-2-methyl-imidazo[1,2-a]pyrazine

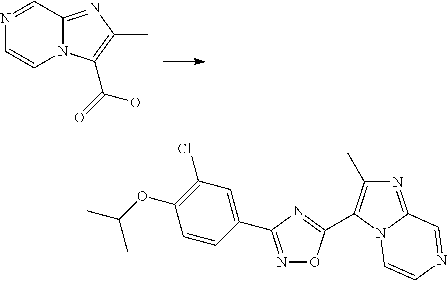

A solution of 2-methylimidazo[1,2-a]pyrazine-3-carboxylic acid (250 mg, 1.411 mmol) in DCE (5 mL) was treated with Hunig's Base (0.542 mL, 3.10 mmol) and HATU (590 mg, 1.552 mmol) at RT for about 15 min. and about 40° C. for about 30 min. The reaction was concentrated and the residue was dissolved in acetic acid (10 mL) and heated at about 100° C. for about 45 min. The reaction was cooled to RT and concentrated under reduced pressure. The residue was partitioned between saturated sodium carbonate solution (10 mL) and methylene chloride (2×10 mL). The organic layers were dried with sodium sulfate, filtered and concentrated under reduced pressure. The residue was purified on silica gel using 9:1/CH₂Cl₂:MeOH. The product fractions were combined and concentrated under reduced pressure to yield 3-[3-(3-chloro-4-isopropoxy-phenyl)-[1,2,4]oxadiazol-5-yl]-2-methyl-imidazo[1,2-a]pyrazine (133 mg, 25%) as a tan solid.

LC/MS (Table 1, Method a) Rₜ=4.31 min, m/z 370.25 (M+H)⁺; ¹H NMR (400 MHz, DMSO-d6) δ 9.43 (d, d, 1H), 9.26 (d, 1H), 8.30 (d, 1H), 8.21 (d, 1H), 8.10 (d, d, 1H), 7.40 (d, 1H), 4.84 (m, 1H), 2.84 (s, 3H), 1.36 (d, 6H)

### Preparation #24a

### Preparation of 3-(3-chloro-4-isopropoxyphenyl)-5-(4-((2,2-dimethyl-1,3-dioxolan-4-yl)methoxy)phenyl)-1,2,4-oxadiazole

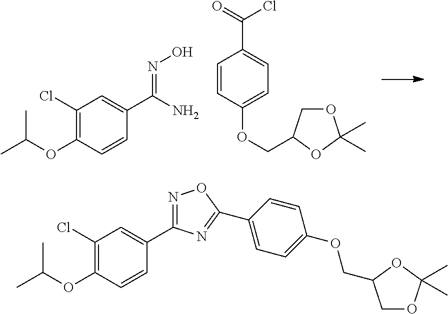

In a 25 mL microwave tube 4-((2,2-dimethyl-1,3-dioxolan-4-yl)methoxy)benzoyl chloride (0.483 g, 1.784 mmol) and (Z)-3-chloro-N′-hydroxy-4-isopropoxybenzimidamide (0.272 g, 1.189 mmol) in pyridine (15 mL) were combined to give an orange solution. The vessel was capped and the reaction heated at about 200° C. for about 20 min under microwave irradiation (Biotage Optimizer™, 300 W). The mixture was cooled, the solvent was removed to afford a yellow solid, which was partitioned between water (100 mL) and EtOAc (50 mL), extracted by EtOAc (2×30 mL), the combined EtOAC layer was washed by water (2×30 mL), and concentrated to afford a yellow solid, which was purified via silica gel chromatography (40 g, 30% EtOAc:Heptane) to afford 3-(3-chloro-4-isopropoxyphenyl)-5-(4-((2,2-dimethyl-1,3-dioxolan-4-yl)methoxy)phenyl)-1,2,4-oxadiazole (0.3 g, 0.674 mmol, 56.7% yield) as white solid. LC/MS (30_{—}95 NH4OAc 4m GC8.olp) Rₜ=3.22 min.; MS m/z: 445.31 (M+H)⁺. ¹H NMR (400 MHz, d-DMSO [symbol] ppm 8.17-8.09 (m, 2H), 8.05 (d, J=2.13 Hz, 1H), 7.99 (dd, J=8.64, 2.15 Hz, 1H), 7.38 (d, J=9.01 Hz, 1H), 7.26-7.19 (m, 2H), 4.88-4.77 (m, 1H), 4.45 (s, 1H), 4.23-4.07 (m, 3H), 3.79 (dd, J=8.42, 6.29 Hz, 1H), 1.35 (m, 12H).

### Preparation #25

### tert-butyl 2-(4-(chlorocarbonyl)phenoxy)acetate

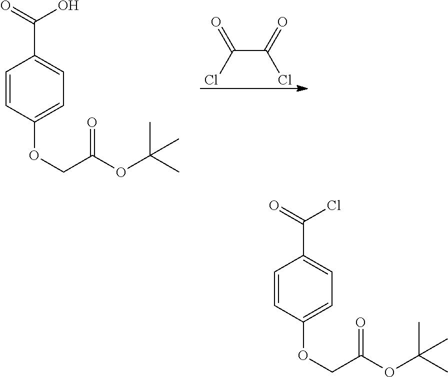

In a 100 mL round bottomed flask was 4-(2-tert-butoxy-2-oxoethoxy)benzoic acid (0.76 g, 3.01 mmol) in dichloromethane (30.1 mL) to give a colorless suspension. Five drops DMF was added to the solution. The reaction mixture was cooled by ice-bath. Oxalyl chloride (0.396 mL, 4.52 mmol) was added dropwise. The ice-bath was removed, and the solution was stirred at RT for about 40 min. The reaction mixture was concentrated to afford tert-butyl 2-(4-(chlorocarbonyl)phenoxy)acetate (0.86 g, 3.18 mmol, 105% yield) as colorless oil. ¹H NMR (400 MHz, CDCl3) d ppm 8.10 (d, 2H), 6.95 (d, 2H), 4.61 (s, 2H), 1.49 (s, 9H)

### Example #29

### Preparation of 2-(4-(3-(3-chloro-4-isopropoxyphenyl)-1,2,4-oxadiazol-5-yl)phenoxy)acetic acid

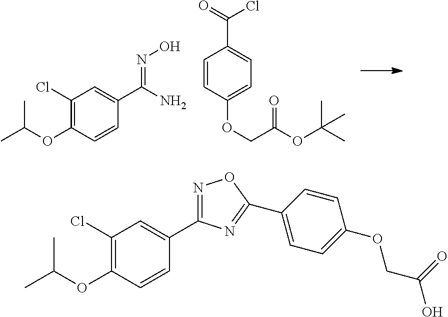

A 25 mL microwave reaction vial was charged with tert-butyl 2-(4-(chlorocarbonyl)phenoxy)acetate (0.815 g, 3.01 mmol) and pyridine (15 mL), (Z)-3-chloro-N′-hydroxy-4-isopropoxybenzimidamide (0.459 g, 2.007 mmol) was added. The vessel was capped and the reaction heated at about 200° C. for about 20 min under microwave irradiation (Biotage Optimizer, 300 W). The mixture was cooled, the reaction mixture was poured into stirring HCl (10%, 100 mL), the resulting suspension was filtered, the solid was washed by HCl (5%, 2×10 mL) and dried to afford grey solid, which was purified by RP-HPLC (A=50 mM ammonium acetate, B=ACN; 30-95% B over 25.0 min (21.0 mL/min flow rate); 21.2×250 mm Thermo Hyperprep C18 column, 8 μm particles) to give 2-(4-(3-(3-chloro-4-isopropoxyphenyl)-1,2,4-oxadiazol-5-yl)phenoxy)acetic acid (0.246 g, 0.633 mmol, 31.5% yield) as white solid. LC/MS (Table 1, Method f) Rₜ=2.08 min.; MS m/z: 389.14 (M+H)⁺.

1H NMR (400 MHz, d-DMSO [symbol] ppm 13.28-13.07 (m, 1H), 8.13 (d, J=9.03 Hz, 2H), 8.05 (d, J=2.13 Hz, 1H), 7.99 (dd, J=8.64, 2.15 Hz, 1H), 7.38 (d, J=9.04 Hz, 1H), 7.18 (d, J=9.06 Hz, 2H), 4.85 (s, 3H), 1.35 (d, J=6.03 Hz, 6H).

### Preparation #26

### Preparation of 5-(6-(1H-benzo[d][1,2,3]triazol-1-yloxy)pyridin-3-yl)-3-(3-chloro-4-isopropoxyphenyl)-1,2,4-oxadiazole

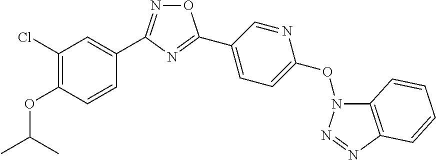

A 25 mL microwave reaction vial was charged with (Z)-3-chloro-N′-hydroxy-4-isopropoxybenzimidamide (0.1 g, 0.437 mmol), 6-bromonicotinic acid (0.097 g, 0.481 mmol), and DCC (0.099 g, 0.481 mmol) in ACN (2.403 mL). HOBT (0.074 g, 0.481 mmol) was added in one portion, the resulting suspension was allowed to stir at RT for about 10 min. DIEA (0.168 mL, 0.962 mmol) was added dropwise, the reaction mixture was heated at about 120° C. for about 30 min under microwave irradiation (Biotage Optimizer, 300 W). The solution was cooled, the reaction mixture was partitioned between EtOAc (50 mL) and water (50 mL), the organic layer was washed by water (2×50 mL), and concentrated afforded yellow solid, which was purified via silica gel chromatography (12 g, 20% EtOAc:Heptane) to afford 5-(6-(1H-benzo[d][1,2,3]-triazol-1-yloxy)pyridin-3-yl)-3-(3-chloro-4-isopropoxyphenyl)-1,2,4-oxadiazole (0.128 g, 0.285 mmol, 65.2% yield) as a white solid.

LC/MS (Table 1, Method a) Rₜ=3.74 min.; MS m/z: 449.18 (M+H)⁺. 1H NMR (400 MHz, d-DMSO) ppm 8.88 (dd, J=2.25, 0.65 Hz, 1H), 8.62 (dd, J=8.68, 2.27 Hz, 1H), 8.15 (t, J=5.28 Hz, 2H), 7.97 (dd, J=8.62, 2.14 Hz, 1H), 7.55 (d, J=0.96 Hz, 1H), 7.52-7.44 (m, 2H), 7.36 (dd, J=8.68, 0.70 Hz, 1H), 7.03 (d, J=8.87 Hz, 1H), 4.73-4.61 (m, 1H), 1.46-1.40 (m, 6H).

### Preparation #27

### (Z)-3-bromo-N′-hydroxy-4-isopropoxybenzimidamide

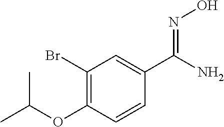

3-Bromo-4-isopropoxybenzonitrile (0.68 g, 2.83 mmol) and hydroxylamine (0.208 mL, 3.12 mmol) were combined in EtOH (20 mL). The reaction mixture was heated at about 65° C. for about 16 h. The reaction mixture was concentrated to afford (Z)-3-bromo-N′-hydroxy-4-isopropoxybenzimidamide (0.76 g, 2.78 mmol, 98% yield) as pale yellow solid. LC/MS (Table 1, Method a) Rₜ=2.89 min.; MS m/z: 275.00 (M+H)⁺.

### Preparation #28

### Preparation of 4-(3-(3-bromo-4-isopropoxyphenyl)-1,2,4-oxadiazol-5-yl)benzonitrile

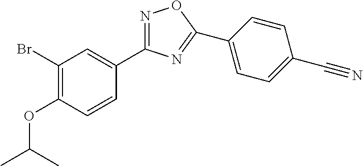

A 25 mL microwave vial equipped with a stirring bar was charged with 4-cyanobenzoyl chloride (0.4 g, 2.416 mmol), (Z)-3-bromo-N′-hydroxy-4-isopropoxybenzimidamide (0.5 g, 1.831 mmol) and pyridine (15 mL) to give an orange solution. The vessel was capped and the reaction heated at about 200° C. for about 20 min under microwave irradiation (Biotage Optimizer, 300 W). The solution was cooled, the reaction mixture was partitioned between aqueous HCl (10%, 150 mL) and DCM (40 mL) mixture, the DCM layer was drained, and the aqueous layer was extracted by DCM (2×20 mL). The combined DCM layers were washed by water (2×20 mL) and concentrated to afford white solid, which was purified via silica gel chromatography (40 g, 40% EtOAc:Heptane) to afford 4-(3-(3-bromo-4-isopropoxyphenyl)-1,2,4-oxadiazol-5-yl)benzonitrile (0.638 g, 1.660 mmol, 91% yield) as white solid. LC/MS (Method c) Rₜ=3.17 min.; MS m/z: 386.19 (M+H)⁺. 1H NMR (400 MHz, d-DMSO) ppm 8.40-8.32 (m, 2H), 8.23 (d, J=2.13 Hz, 1H), 8.14 (dd, J=8.14, 0.61 Hz, 2H), 8.05 (dd, J=8.65, 2.15 Hz, 1H), 7.36 (d, J=9.12 Hz, 1H), 4.89-4.77 (m, 1H), 1.35 (d, J=6.03 Hz, 6H).

### Preparation #29

### Preparation of 4-(3-(3-bromo-4-isopropoxyphenyl)-1,2,4-oxadiazol-5-yl)benzaldehyde

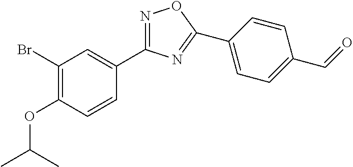

A 100 mL round bottom flask equipped with septa cap outfitted with nitrogen inlet needle was charged with 4-(3-(3-bromo-4-isopropoxyphenyl)-1,2,4-oxadiazol-5-yl)benzonitrile (0.64 g, 1.666 mmol) in DCM (33.3 mL) to give a colorless solution. The reaction mixture was cooled to about −40° C. by ACN-dry ice bath and it turned into a white suspension. Dibal-H (3.33 mL, 3.33 mmol) was added dropwise over about 10 min. It was stirred for an extra about 60 min at about −40° C. Methanol (0.135 mL, 3.33 mmol) was added dropwise to quench the reaction. Then all of the mixture was poured into stirring Rochelle's salt (200 mL). It was stirred at RT for 4 h, then partitioned, the aqueous layer was extracted by DCM (2×50 mL), the combined DCM layers were washed by water (60 mL), dried over MgSO₄. Filtration and concentration afforded 1.04 g orange oil, which was purified via silica gel chromatography (40 g, 40% EtOAc:Heptane) to afford 4-(3-(3-bromo-4-isopropoxyphenyl)-1,2,4-oxadiazol-5-yl)benzaldehyde (0.551 g, 1.423 mmol, 85% yield) as pale yellow solid. LC/MS (Method c)

Rₜ=3.17 min.; MS m/z: 388.94 (M+H)⁺. 1H NMR (400 MHz, d-DMSO) ppm 10.15 (s, 1H), 8.41 (d, J=8.20 Hz, 2H), 8.24 (d, J=2.13 Hz, 1H), 8.20-8.14 (m, 2H), 8.06 (dd, J=8.64, 2.15 Hz, 1H), 7.37 (d, J=9.11 Hz, 1H), 4.89-4.78 (m, 1H), 1.36 (d, J=6.03 Hz, 6H).

### Preparation #30

### Preparation of 3-(3-bromo-4-isopropoxyphenyl)-5-(4-(dimethoxymethyl)phenyl)-1,2,4-oxadiazole

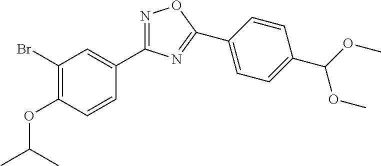

4-(3-(3-Bromo-4-isopropoxyphenyl)-1,2,4-oxadiazol-5-yl)benzaldehyde (0.551 g, 1.423 mmol), molecular sieve (4 Å, 8-12 mesh, 130 mg) and p-toluenesulfonic acid monohydrate (0.037 g, 0.195 mmol) were added in trimethyl orthoformate (4 mL, 36.2 mmol) and methanol (6 mL), the reaction mixture was heated at about 80° C. for about 16 h. The solution was cooled, the reaction mixture was concentrated to afford grey solid, which was purified via silica gel chromatography (12 g, 20% EtOAc:Heptane) to afford 3-(3-bromo-4-isopropoxyphenyl)-5-(4-(dimethoxymethyl)phenyl)-1,2,4-oxadiazole (0.61 g, 1.366 mmol, 96% yield) as white solid. LC/MS (Table 1, Method a) Rₜ=3.31 min.; MS m/z: 435.03 (M+H)⁺. 1H NMR (400 MHz, d-DMSO) ppm 8.25-8.19 (m, 3H), 8.05 (dd, J=8.63, 2.14 Hz, 1H), 7.67 (d, J=8.18 Hz, 2H), 7.35 (d, J=9.02 Hz, 1H), 5.52 (s, 1H), 4.86-4.78 (m, 1H), 3.30 (s, 6H), 1.35 (d, J=6.02 Hz, 7H).

### Preparation #31

### Preparation of 5-(5-(4-(dimethoxymethyl)phenyl)-1,2,4-oxadiazol-3-yl)-2-isopropoxybenzonitrile

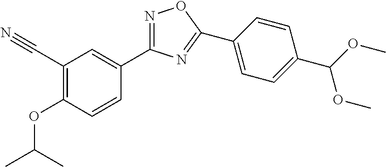

A 25 mL microwave vial equipped with a stirring bar was charged with 3-(3-bromo-4-isopropoxyphenyl)-5-(4-(dimethoxymethyl)phenyl)-1,2,4-oxadiazole (0.25 g, 0.577 mmol), copper(I) cyanide (0.133 g, 1.485 mmol) and pyridine (15 mL). The vessel was capped and the reaction heated to about 230° C. for about 30 min under microwave irradiation (Biotage Optimizer, 300 W). The solution was cooled and the reaction mixture was concentrated. To the residue was added hydrated ferric chloride (0.8 g), concentrated hydrochloric acid (2 mL) and water (12 mL). The solution was heated at about 65° C. for about 20 min, the aqueous mixture was extracted by DCM (3×30 mL), the combined DCM layers were washed with FeCl₃ solution (2×20 mL), then water (2×20 mL), dried (brine, MgSO₄) and concentrated to yield yellow solid, which was purified via silica gel chromatography (40 g, 20% EtOAc:Heptane) to afford 5-(5-(4-(dimethoxymethyl)phenyl)-1,2,4-oxadiazol-3-yl)-2-isopropoxybenzonitrile (0.086 g, 0.227 mmol, 39.3% yield) as pale yellow solid which was used in the next step without further purification.

### Preparation #32

### Preparation of 5-(5-(4-formylphenyl)-1,2,4-oxadiazol-3-yl)-2-isopropoxybenzonitrile

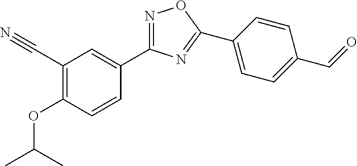

5-(5-(4-(Dimethoxymethyl)phenyl)-1,2,4-oxadiazol-3-yl)-2-isopropoxybenzonitrile (0.086 g, 0.227 mmol) and p-toluenesulfonic acid monohydrate (0.043 g, 0.227 mmol) were added in acetone (10 mL) to give a colorless solution. The reaction mixture was heated at about 60° C. for about 2 h. The solution was cooled, the reaction mixture was concentrated, the residue was purified via silica gel chromatography (12 g, 50% EtOAc:Heptane) to afford 5-(5-(4-formylphenyl)-1,2,4-oxadiazol-3-yl)-2-isopropoxybenzonitrile (0.077 g, 0.231 mmol, 102% yield) as white solid. LC/MS (Table 1, Method f) Rₜ=2.88 min.; MS m/z: 334.08 (M+H)⁺.

### Example #30

### Preparation of 1-(4-(3-(3-cyano-4-isopropoxyphenyl)-1,2,4-oxadiazol-5-yl)benzyl)azetidine-3-carboxylic acid

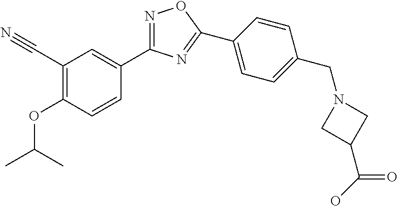

5-(5-(4-Formylphenyl)-1,2,4-oxadiazol-3-yl)-2-isopropoxybenzonitrile (0.077 g, 0.231 mmol) and azetidine-3-carboxylic acid (0.028 g, 0.277 mmol) were combined in methanol (11.55 mL) and DCE (11.55 mL) in a sealed vial. Acetic acid (0.066 mL, 1.155 mmol) was added. The reaction mixture was stirred at ambient temperature for about 2 h. MP-cyanoborohydride (0.265 g, 0.570 mmol) was added and the reaction stirred for about 24 h. The solution was filtered, the solid was washed with methylene chloride and methanol, and the filtrate was concentrated to afford a white solid, which was recrystallized by methanol (5 mL) to give 1-(4-(3-(3-cyano-4-isopropoxyphenyl)-1,2,4-oxadiazol-5-yl)benzyl)azetidine-3-carboxylic acid (0.025 g, 0.060 mmol, 25.9% yield) as white solid: LC/MS (Table 1, Method a) Rₜ=2.10 min.; MS m/z: 420.26 (M+H)⁺. 1H NMR (400 MHz, d-DMSO) ppm 8.35-8.28 (m, 2H), 8.17-8.11 (d, J=8.00 Hz, 2H), 7.56-7.50 (m, 8.69 Hz, 3H), 4.98-4.89 (m, 1H), 3.68 (s, 2H), 3.43 (s, 2H), 3.25-3.23 (m, 3H), 1.38 (d, J=6.03 Hz, 6H).

### Example #31

### Preparation of 1-(4-(3-(3-chloro-4-isopropoxyphenyl)-1,2,4-oxadiazol-5-yl)phenyl)cyclopropanecarbonitrile

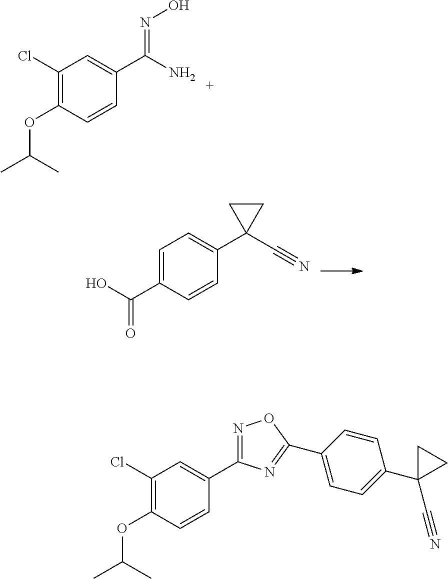

A 20 mL microwave vial was charged with 4-(1-cyanocyclopropyl)benzoic acid (720 mg, 3.85 mmol), (Z)-3-chloro-N′-hydroxy-4-isopropoxybenzimidamide (880 mg, 3.85 mmol), DCC (873 mg, 4.23 mmol), HOBT (648 mg, 4.23 mmol), ACN (10 mL), and DIEA (1.478 mL, 8.46 mmol). The vial was capped and heated to about 160° C. via microwave irradiation for about 25 min (max 300 W). Solvent was removed under reduced pressure and crude oil was purified by flash column chromatography (Analogix® system, heptane/ethyl acetate, 0-45% ethyl acetate over 30 min; 80 g column, 60 mL/min flow rate). Fractions containing product were combined, rotovapped, and dried in a vacuum oven overnight to give 1-(4-(3-(3-chloro-4-isopropoxyphenyl)-1,2,4-oxadiazol-5-yl)phenyl)cyclopropanecarbonitrile (347 mg, 23.8%) as a yellow solid. LC/MS (Table 1, Method c) Rₜ=3.19 min, m/z 380.43 (M+H)⁺; ¹H NMR (400 MHz, DMSO)) δ ppm 8.22-8.12 (m, 2H), 8.05 (d, 1H), 7.99 (dd, 2.14 Hz, 1H), 7.62-7.55 (m, 2H), 7.38 (d, 1H), 4.82 (td, 1H), 1.90 (q, 2H), 1.67 (q, 2H), 1.38-1.33 (m, 6H).

### Preparation #32a

### Preparation of 1-(4-(3-(3-chloro-4-isopropoxyphenyl)-1,2,4-oxadiazol-5-yl)phenyl)cyclopropanecarbaldehyde

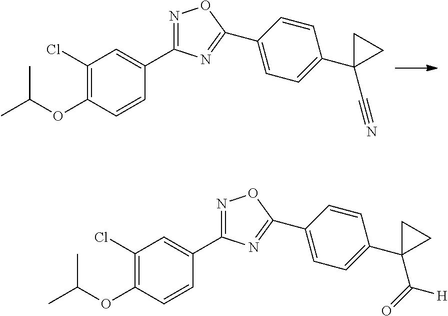

A 100 mL round bottom flask was charged with 1-(4-(3-(3-chloro-4-isopropoxyphenyl)-1,2,4-oxadiazol-5-yl)phenyl)cyclopropanecarbonitrile (300 mg, 0.790 mmol) and dichloromethane (8 mL) and then cooled to about −40° C. Dibal-H (0.869 mL, 0.869 mmol) was added slowly via syringe and the reaction mixture left to warm to RT overnight. The reaction was quenched by addition of MeOH (4 mL), and aqueous Rochelle's salt (4 mL). Layers were separated and the aqueous layer was extracted with DCM (3×25 mL). Organic layers were washed with saturated sodium bicarbonate solution, then dried over MgSO₄, and concentrated. To a solution of the crude material in 3 mL of THF was added 3 mL of 1N HCl. The mixture was stirred at RT for about 1 h. The mixture was rotovapped to remove THF. The material was then purified via flash column chromatography (Analogix®, 40 g column, 0-40% ethyl acetate in heptane over 30 min, 30 mL/min flow rate). The fractions containing product were combined and concentrated to give 1-(4-(3-(3-chloro-4-isopropoxyphenyl)-1,2,4-oxadiazol-5-yl)phenyl)cyclopropanecarbaldehyde (144 mg, 48%) as a tacky yellow solid. LC/MS (Table 1, Method c) Rₜ=3.11 min, m/z 383.50 (M+H)⁺.

### Example #33

### Preparation of 3-((1-(4-(3-(3-chloro-4-isopropoxyphenyl)-1,2,4-oxadiazol-5-yl)phenyl)cyclopropyl)methylamino)propanoic acid, Trifluoroacetic Acid

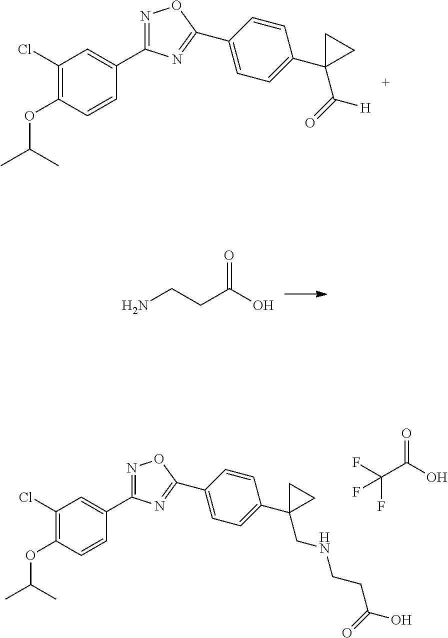

A 20 mL vial was charged with 1-(4-(3-(3-chloro-4-isopropoxyphenyl)-1,2,4-oxadiazol-5-yl)phenyl)cyclopropanecarbaldehyde (46 mg, 0.120 mmol), methanol (2.5 mL), 3-aminopropanoic acid (10.70 mg, 0.120 mmol) and acetic acid (0.034 mL, 0.601 mmol). The vial was capped and the mixture stirred for about 30 min at RT. Next, sodium cyanoborohydride (7.55 mg, 0.120 mmol) was added in one aliquot and the reaction was stirred overnight at RT. Solvents were removed under reduced pressure and the crude material was purified via RP-HPLC (A=0.1% TFA, B=ACN; 30% to 95% B over 30 min at 21.0 mL/min; UVλ=254 nm; Thermo Hyperprep HS C18, 8 250×21.2 mm column). Fractions containing product were rotovapped and lyophilized to give 3-((1-(4-(3-(3-chloro-4-isopropoxyphenyl)-1,2,4-oxadiazol-5-yl)phenyl)cyclopropyl)methylamino)propanoic acid (27 mg, 40%) as the trifluoroacetic acid salt. LC/MS (Table 1, Method c) Rₜ=2.07 min, m/z 456.25 (M+H)⁺; ¹H NMR (400 MHz, methanol) δ ppm 8.22 (d, 2H), 8.11 (d, 1H), 8.03 (dd, 1H), 7.69 (d, J=8.19 Hz, 2H), 7.24 (d, 1H), 4.80-4.76 (m, 1H), 3.36 (s, 2H), 3.13 (t, 2H), 2.44 (t, 2H), 1.40 (d, 6H), 1.17 (d, 4H).

### Example #34

### Preparation of N-(4-(3-(3-chloro-4-isopropoxyphenyl)-1,2,4-oxadiazol-5-yl)benzyl)-1-(2,2-dimethyl-1,3-dioxolan-4-yl)methanamine

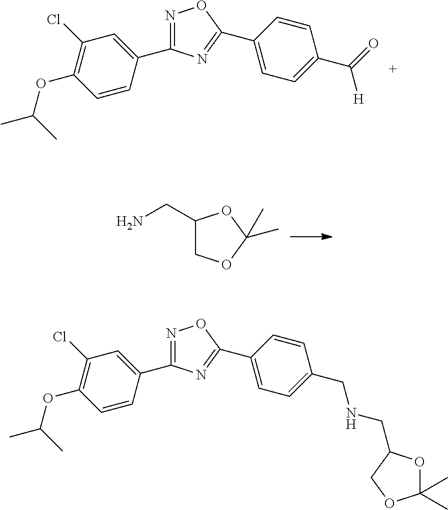

4-(3-(3-chloro-4-isopropoxyphenyl)-1,2,4-oxadiazol-5-yl)benzaldehyde (150 mg, 0.438 mmol), (2,2-dimethyl-1,3-dioxolan-4-yl)methanamine (0.057 mL, 0.438 mmol), methanol (4 mL), and acetic acid (0.125 mL, 2.188 mmol) were loaded into a 25 mL flask equipped with a stirring bar. The mixture was stirred for about 10 min at RT under nitrogen. Sodium cyanoborohydride (27.5 mg, 0.438 mmol) was added in one portion, and the reaction mixture was stirred at RT overnight. Solvent was removed under reduced pressure and crude material was purified by RP-HPLC (A=50 mM ammonium acetate, B=ACN; 40% to 80% B over 30 min at 21.0 mL/min; UVλ=254 nm; Thermo Hyperprep HS C18, 8 μm, 250×21.2 mm column). Fractions containing product were combined, rotovapped and lyophilized to give N-(4-(3-(3-chloro-4-isopropoxyphenyl)-1,2,4-oxadiazol-5-yl)benzyl)-1-(2,2-dimethyl-1,3-dioxolan-4-yl)methanamine (130.9 mg, 64.7%) as a white solid. LC/MS (Table 1, Method c) Rₜ=2.59 min, m/z 458.62 (M+H)⁺; ¹H NMR (400 MHz, DMSO) δ ppm 8.13 (d, 2H), 8.06 (d, 1H), 8.00 (dd, 1H), 7.61 (d, 2H), 7.39 (d, 1H), 4.82 (sept, 1H), 4.15 (p, 1H), 3.99 (dd, 1H), 3.84 (s, 2H), 3.63 (dd, 1H), 2.61 (ddd, 2H), 1.86 (s, 4H), 1.35 (d, 6H) 1.26 (s, 3H).

### Example #35

### Preparation of 3-(4-(3-(3-chloro-4-isopropoxyphenyl)-1,2,4-oxadiazol-5-yl)benzylamino)propane-1,2-diol

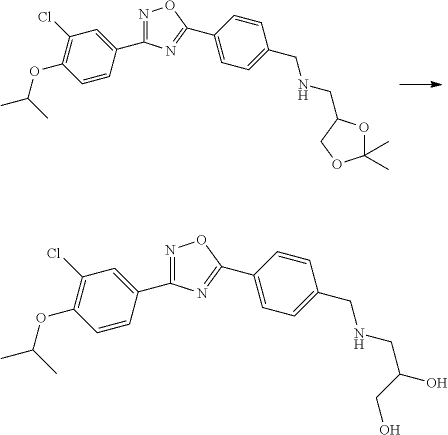

To a solution of N-(4-(3-(3-chloro-4-isopropoxyphenyl)-1,2,4-oxadiazol-5-yl)benzyl)-1-(2,2-dimethyl-1,3-dioxolan-4-yl)methanamine (108 mg, 0.236 mmol) in THF (4 mL) was added 1N aqueous HCl (0.778 mL, 0.778 mmol) The reaction was heated to about 65° C. under nitrogen for about 90 min. Heating was stopped and the reaction was neutralized by addition of 1N aqueous NaOH (0.778 mL, 0.778 mmol). THF was removed under reduced pressure and the remaining aqueous solution was basified (pH approx 9) by the addition of 0.1N NaOH, at which point white precipitate formed. Solid was collected by vacuum filtration, and washed with 0.1N NaOH (3×10 mL). Solid was dried in a vacuum oven overnight to give 3-(4-(3-(3-chloro-4-isopropoxyphenyl)-1,2,4-oxadiazol-5-yl)benzylamino)propane-1,2-diol (31.7 mg, 32%) as an off-white solid. LC/MS (Table 1, Method c) Rₜ=1.90 min, m/z 418.47 (M+H)⁺; ¹H NMR (400 MHz, methanol) δ ppm 7.22 (d, J=8.68 Hz, 1H), 7.60 (d, 2H), 8.01 (dd, 1H), 8.10 (d, 1H), 8.16 (d, 2H), 4.78 (sept, 1H), 2.76 (dd, 1H), 2.63 (dd, 1H), 3.52 (d, 2H), 3.90 (d, 2H), 3.78 (m, 1H), 1.40 (d, 6H).

### Preparation #33

### Preparation of (Z)-methyl 3-(4-(3-(3-chloro-4-isopropoxyphenyl)-1,2,4-oxadiazol-5-yl)phenyl)acrylate

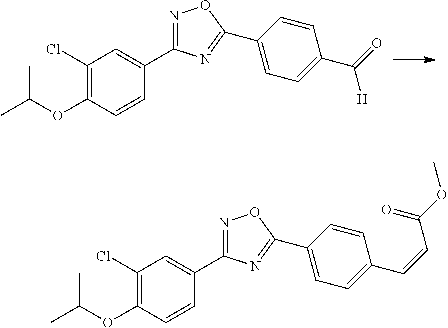

A two-neck round bottom flask was charged with methyl 2-(bis(2,2,2-trifluoroethoxy)phosphoryl)acetate (0.235 mL, 1.109 mmol), 18-crown-6 (1465 mg, 5.54 mmol) and THF (15 mL). The mixture was then cooled to about −78° C. under an atmosphere of nitrogen. Potassium bis(trimethylsilyl)amide (221 mg, 1.109 mmol) was added and the mixture stirred for a few min. 4-(3-(3-chloro-4-isopropoxyphenyl)-1,2,4-oxadiazol-5-yl)benzaldehyde (380 mg, 1.109 mmol) was added and the mixture stirred at about −78° C. for about 90 min and then left to warm to RT overnight. Reaction was quenched by the addition of saturated NH₄C1 (aqueous). The mixture was separated and the aqueous layer was extracted with ether (3×10 mL). The combined organics were dried over MgSO₄ and concentrated to give an off-white solid. The solid was triturated with MeOH and collected by vacuum filtration and washed with MeOH (3×10 mL). The collected solid was dried overnight in a vacuum oven to give (Z)-methyl 3-(4-(3-(3-chloro-4-isopropoxyphenyl)-1,2,4-oxadiazol-5-yl)phenyl)acrylate (325 mg, 73.5%).

LC/MS (Table 1, Method c) Rₜ=3.22 min, m/z 399.16 (M+H)⁺. ¹H NMR (400 MHz, DMSO) δ ppm 8.18 (d, 2H), 8.06 (d, 1H), 8.01 (dd, 1H), 7.79 (d, 2H), 7.40 (d, 1H), 7.18 (d, 1H), 6.84 (d, 1H), 6.20 (d, 1H), 4.83 (sept, 1H), 3.67 (s, 3H), 1.35 (d, 6H).

### Example #36

### Preparation of Trans-methyl 2-(4-(3-(3-chloro-4-isopropoxyphenyl)-1,2,4-oxadiazol-5-yl)phenyl)cyclopropanecarboxylate

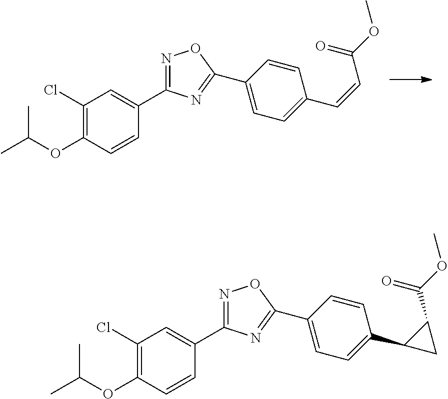

To a stirred suspension of trimethylsulfoxonium iodide (234 mg, 1.065 mmol) in DMSO (5.0 mL) under nitrogen, was added, in portions NaH (42.6 mg, 1.065 mmol), with a water bath in place to keep the reaction between about 25-30° C. Upon completion of hydrogen evolution, a solution of (Z)-methyl 3-(4-(3-(3-chloro-4-isopropoxyphenyl)-1,2,4-oxadiazol-5-yl)phenyl)acrylate (386 mg, 0.968 mmol) in DMSO (5.00 mL) was added drop-wise, keeping the reaction temperature at or below about 35° C. After addition was complete, the reaction was stirred at RT for about an hour and a half and then warmed to about 50° C. for about 2 hs. 50 mL of water was then added to the reaction, and the reaction left to stir at RT overnight. The reaction mixture was diluted with saturated aqueous sodium chloride, and the aqueous layer was extracted 3× with 75 mL EtOAc. Organic layers were combined, dried over MgSO₄, and concentrated. The crude material was purified by RP-HPLC (A=50 mM ammonium acetate, B=ACN; 30% to 100% B over 30 min at 21.0 mL/min; UVλ=254 nm; Thermo Hyperprep HS C18, 8 μm, 250×21.2 mm column). Fractions containing product were combined, concentrated and lyophilized to give trans-methyl 2-(4-(3-(3-chloro-4-isopropoxyphenyl)-1,2,4-oxadiazol-5-yl)phenyl)cyclopropanecarboxylate (155 mg, 39%) as a white solid. LC/MS (Table 1, Method c) Rₜ=3.27 min, m/z 413.17 (M+H)⁺. ¹H NMR (400 MHz, DMSO) δ ppm 8.08 (d, 2H), 8.06 (d, 1H), 7.99 (dd, 1H), 7.47 (d, 2H), 7.39 (d, 1H), 4.82 (sept, 1H), 3.66 (s, 3H), 2.59 (ddd, 1H), 2.12 (ddd, 1H), 1.58 (ddd, 1H), 1.53 (ddd, 1H), 1.35 (d, 6H).

### Example #37

### Preparation of Trans-2-(4-(3-(3-chloro-4-isopropoxyphenyl)-1,2,4-oxadiazol-5-yl)phenyl)cyclopropanecarboxylic acid

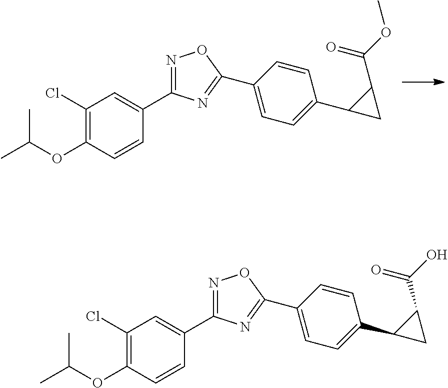

To a suspension of (1S,2S)-methyl 2-(4-(3-(3-chloro-4-isopropoxyphenyl)-1,2,4-oxadiazol-5-yl)phenyl)cyclopropane carboxylate (111 mg, 0.269 mmol) in ethanol (5 mL) was added 2 N NaOH (5 mL, 10.00 mmol). The mixture was stirred under nitrogen at RT overnight. Reaction mixture was neutralized by addition of acetic acid, and then acidified with a few drops of 1 N aqueous HCl (pH about 2). White solid precipitated and was collected by filtration, washed with 0.1 N HCl (3×5 mL), and dried under vacuum to give trans-2-(4-(3-(3-chloro-4-isopropoxyphenyl)-1,2,4-oxadiazol-5-yl)phenyl)cyclopropanecarboxylic acid (64 mg, 59%). LC/MS (Table 1, Method f) Rₜ=2.99 min, m/z 399.16 (M+H)⁺. ¹H NMR (400 MHz, DMSO) δ ppm 8.07 (d, 2H), 8.05 (d, 1H), 7.99 (dd, 1H), 7.45 (d, 2H), 7.38 (d, 1H), 4.82 (sept., 1H), 2.54 (m, 1H), 1.97 (m, 1H), 1.53 (td, 1H), 1.46 (ddd, 1H), 1.35 (d, 6H).

### Example #38

### Preparation of tert-butyl 5-(3-(3-chloro-4-isopropoxyphenyl)-1,2,4-oxadiazol-5-yl)isoindoline-2-carboxylate

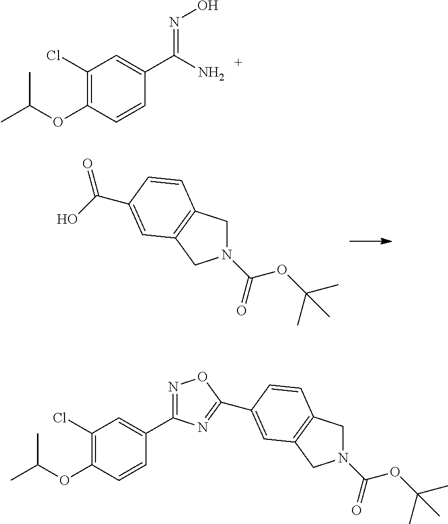

To a solution of 2-(tert-butoxycarbonyl)isoindoline-5-carboxylic acid (190 mg, 0.722 mmol) in ACN (3 mL) in a 5 mL microwave vial was added HOBT (330 mg, 2.16 mmol), DCC (298 mg, 2.16 mmol), and DIEA (0.115 mL, 0.656 mmol). The mixture was stirred RT for about 16 h. Next, (Z)-3-chloro-N′-hydroxy-4-isopropoxybenzimidamide (150 mg, 0.656 mmol) (prepared by General Procedure B) was added and the reaction was heated to about 150° C. under microwave irradiation (max 300 W) for about 20 min. After cooling, the reaction mixture was filtered, concentrated, and purified via Analogix® FCC system using RediSep™ 40 g column, with a gradient of 0-40% EtOAc/Heptane over 30 min. at a flow rate of 30 mL/min Fractions containing product were combined, rotovapped, and dried in a vacuum oven to give tert-butyl 5-(3-(3-chloro-4-isopropoxyphenyl)-1,2,4-oxadiazol-5-yl)isoindoline-2-carboxylate (46.2 mg, 15.5%) LC/MS (Table 1, Method c) Rₜ=3.40 min, m/z 456.22 (M+H)⁺; ¹H NMR (400 MHz, DMSO) δ ppm 8.16 (d, 1H), 8.10 (s, 1H), 8.05 (d, 1H), 7.61 (m, 1H), 7.39 (d, 1H), 4.82 (sept, 1H), 4.70 (d, 4H), 1.48 (s, 9H), 1.35 (d, 6H).

### Example #39

### Preparation of 3-(3-chloro-4-isopropoxyphenyl)-5-(isoindolin-5-yl)-1,2,4-oxadiazole, Trifluoroacetic Acid

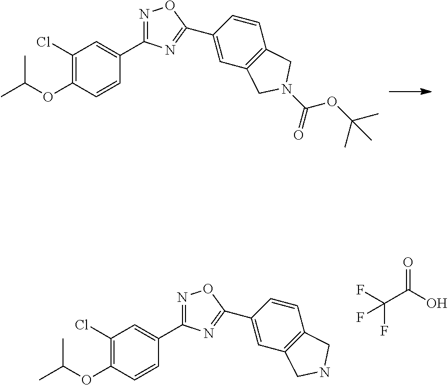

To a solution of tert-butyl 5-(3-(3-chloro-4-isopropoxyphenyl)-1,2,4-oxadiazol-5-yl)isoindoline-2-carboxylate (41 mg, 0.090 mmol) in DCM (2 mL) was added TFA (0.5 mL, 6.49 mmol). The mixture was stirred at RT under nitrogen for about 30 min. After 30 min, ether was added slowly to the mixture, until it became cloudy and a white precipitate formed. The solid was collected by filtration and washed with ether (3×10 mL). The collected solid was then dried in vacuum oven to give 3-(3-chloro-4-isopropoxyphenyl)-5-(isoindolin-5-yl)-1,2,4-oxadiazole as the TFA salt (26.7 mg, 62.6%). LC/MS (Table 1, Method c) Rₜ=2.29 min, m/z 356.17 (M+H)⁺. ¹H NMR (400 MHz, DMSO) δ ppm 9.46 (s, 2H), 8.27 (s, 1H), 8.20 (d, 1H), 8.00 (d, 1H), 7.70 (d, 1H), 7.41 (d, 1H), 4.83 (sept, 1H), 4.64 (d, 4H), 1.35 (d, 6H).

### Preparation #34

### Preparation of methyl 3-(5-(3-(3-chloro-4-isopropoxyphenyl)-1,2,4-oxadiazol-5-yl)isoindolin-2-yl)propanoate

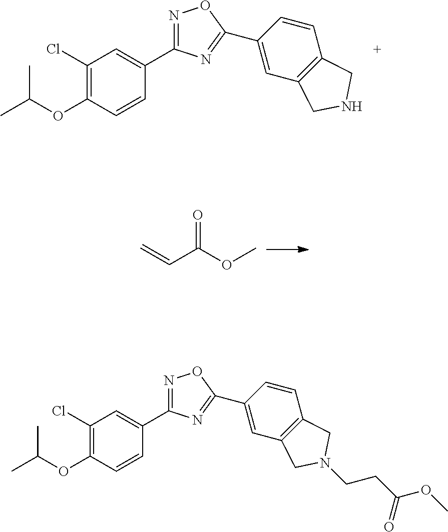

3-(3-Chloro-4-isopropoxyphenyl)-5-(isoindolin-5-yl)-1,2,4-oxadiazole (16.7 mg, 0.047 mmol) was added to a 2 mL microwave vial equipped with a stirring bar. Methyl acrylate (8.45 μL, 0.094 mmol), and methanol (1.0 mL) were added, the vial capped, and the reaction heated to about 90° C. for about 20 min under microwave irradiation (300 W). After about 20 minutes, another aliquot of methyl acrylate (8.45 μL, 0.094 mmol) was added, the vial was re-sealed, and heated to about 110° C. for about 40 min under microwave irradiation (300 W). The reaction was then concentrated and dried under vacuum overnight to give crude methyl 3-(5-(3-(3-chloro-4-isopropoxyphenyl)-1,2,4-oxadiazol-5-yl)isoindolin-2-yl)propanoate as a yellow oil (21.6 mg, 104%). The product was used without further purification. LC/MS (Table 1, Method c) Rₜ=2.85 min, m/z 442.45 (M+H)⁺

### Example #41

### Preparation of 3-(5-(3-(3-chloro-4-isopropoxyphenyl)-1,2,4-oxadiazol-5-yl)isoindolin-2-yl)propanoic acid, Hydrochloric Acid

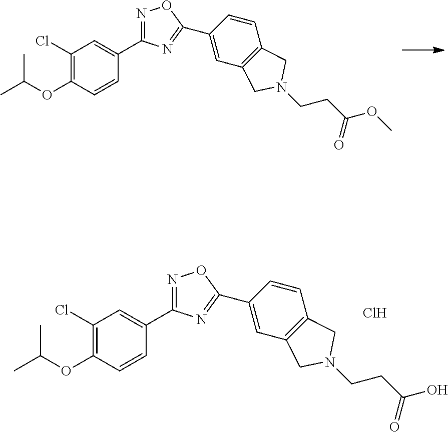

To a solution of methyl 3-(5-(3-(3-chloro-4-isopropoxyphenyl)-1,2,4-oxadiazol-5-yl)isoindolin-2-yl)propanoate (21 mg, 0.048 mmol) in ethanol (1 mL) was added 2 M aqueous NaOH (1 mL, 2.000 mmol). The reaction was stirred at RT under an atmosphere of nitrogen for about 4 h. Reaction mixture was then acidified to about a pH of 1 by addition of 2 N HCl, at which time a precipitate formed. The solid was collected by filtration and washed with water (3×5 mL). The solid was then dried in a vacuum oven overnight to give 3-(5-(3-(3-chloro-4-isopropoxyphenyl)-1,2,4-oxadiazol-5-yl)isoindolin-2-yl)propanoic acid as the hydrochloride salt (10.2 mg, 46.2%). LC/MS (Table 1, Method c) Rₜ=1.86 min, m/z 428.20 (M+H)⁺. ¹H NMR (400 MHz, DMSO) δ ppm 12.12 (m, 1H), 8.23 (s, 1H), 8.19 (d, 1H), 8.07 (d, 1H), 8.01 (dd, 1H), 7.68 (d, 1H), 7.41 (d, 1H), 4.83 (sept, 1H), 4.72 (s, 4H), 3.58 (t, 2H), 2.84 (t, 2H), 1.36 (d, 6H)

### Example #42

### Preparation of (Z)-3-(4-(3-(3-chloro-4-isopropoxyphenyl)-1,2,4-oxadiazol-5-yl)phenyl)acrylic acid

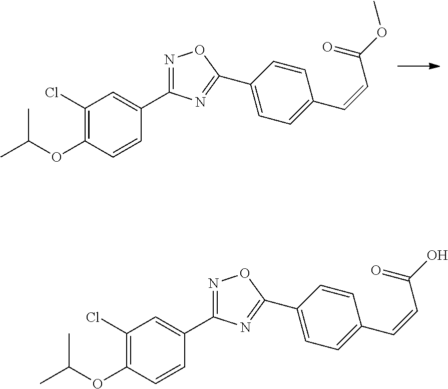

To a solution of (Z)-methyl 3-(4-(3-(3-chloro-4-isopropoxyphenyl)-1,2,4-oxadiazol-5-yl)phenyl)acrylate (30 mg, 0.075 mmol) in EtOH (2 mL) was added 2 N aqueous NaOH (2 mL). The reaction was stirred at RT, under nitrogen, for about 2 h. The reaction was acidified via addition of 1 N HCl, until a precipitate formed. The solid was collected by filtration, washed with 0.2 N HCl, and dried in a vacuum oven to give (Z)-3-(4-(3-(3-chloro-4-isopropoxyphenyl)-1,2,4-oxadiazol-5-yl)phenyl)acrylic acid (8.2 mg, 28.3%). LC/MS (Table 1, Method c) Rₜ=2.64 min, m/z 385.12 (M+H)⁺. ¹H NMR (400 MHz, DMSO) δ ppm 13.11-12.20 (m, 1H), 8.15 (d, 2H), 8.04 (d, 1H), 7.99 (dd, 1H), 7.78 (d, 2H), 7.37 (d, 1H), 7.03 (d, 1H), 6.12 (d, 1H), 4.81 (sept., 1H), 1.33 (d, 6H)

### Preparation #35

### Preparation of 3-chloro-4-(3-(3-chloro-4-isopropoxyphenyl)-1,2,4-oxadiazol-5-yl)aniline

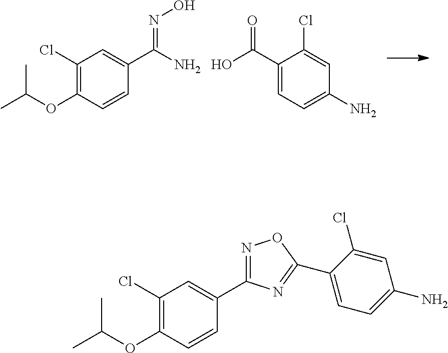

(Z)-3-chloro-N′-hydroxy-4-isopropoxybenzimidamide (0.5 g, 2.187 mmol), 4-amino-2-chlorobenzoic acid (0.413 g, 2.405 mmol), DCC (0.496 g, 2.405 mmol), HOBT (0.368 g, 2.405 mmol) were placed in an 80 mL microwave vial and ACN (12.01 mL) was added. The reaction mixture was stirred for about 5 min at RT before the addition of DIEA (0.840 mL, 4.81 mmol). The reaction mixture was heated to about 120° C. for about 30 min in a microwave. TLC (50% EA/Hept) indicated 4 spots Rf 0.8, 0.6, 0.5 and 0.3. LC/MS (2007_{—}9349) indicated by UV 16% (2.61 mins) to (M+H) 364.31. The solvent was removed and the crude material purified by FCC (50% EA/Hept) to afford 3-chloro-4-(3-(3-chloro-4-isopropoxyphenyl)-1,2,4-oxadiazol-5-yl)aniline (534 mg, 1.466 mmol, 67.1% yield). LC/MS (Table A, Method b) indicated a 99% by UV (3.10 mins) and 92% by ELSD (3.06 mins) (M+H)⁺ 1364.12

### Example #43

### Preparation of 3-(3-chloro-4-(3-(3-chloro-4-isopropoxyphenyl)-1,2,4-oxadiazol-5-yl)phenylamino)cyclobutanecarboxylic acid

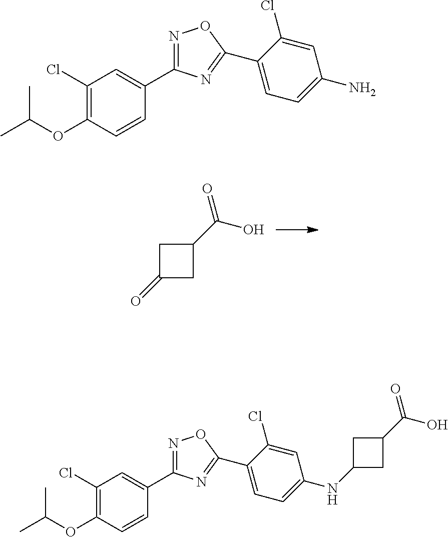

3-Chloro-4-(3-(3-chloro-4-isopropoxyphenyl)-1,2,4-oxadiazol-5-yl)aniline (200 mg, 0.549 mmol) and 3-oxocyclobutanecarboxylic acid (62.7 mg, 0.549 mmol) in methanol (1280 μL) at RT was added acetic acid (842 μl, 14.72 mmol). The reaction mixture was stirred at RT for about 10 min before addition of sodium cyanoborohydride (17.25 mg, 0.275 mmol) as a single portion. The reaction mixture was stirred at RT overnight. The solvent was removed and the crude material purified by FCC (50% EA/Hept) to afford 3-(3-chloro-4-(3-(3-chloro-4-isopropoxyphenyl)-1,2,4-oxadiazol-5-yl)phenylamino)cyclobutanecarboxylic acid (135 mg, 0.292 mmol, 53.2% yield) as a white solid. LC/MS (Table A, Method b) indicated 100% by UV (3.06 mins) to (M+H)⁺ 364.12.

### Preparation #36

### 4-(3-(3-chloro-4-isopropoxyphenyl)-1,2,4-oxadiazol-5-yl)aniline

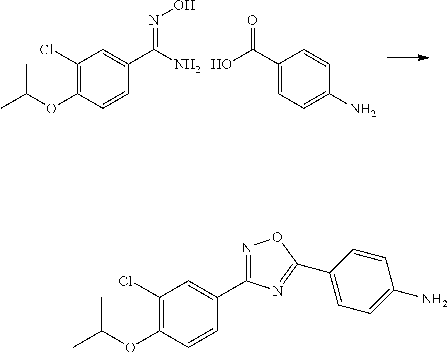

(Z)-3-chloro-N′-hydroxy-4-isopropoxybenzimidamide (1 g, 4.37 mmol), 4-aminobenzoic acid (0.660 g, 4.81 mmol), HOBT (0.737 g, 4.81 mmol), DCC (0.992 g, 4.81 mmol) and DIEA (1.680 mL, 9.62 mmol) were combined in a microwave vial. The reaction mixture was heated in the microwave for about 20 min at about 150° C. The reaction mixture was filtered to remove the urea formed in the reaction and the solvent was removed in vacuo. The crude material was purified by FCC (50% ethyl acetate/heptane) to afford 4-(3-(3-chloro-4-isopropoxyphenyl)-1,2,4-oxadiazol-5-yl)aniline (729 mg, 2.211 mmol, 50.6% yield) as an off white solid: LC/MS (Table A, Method b) 3.00 min, (M+H)⁺ 330.13.

### Example #44

### 3-(4-(3-(3-chloro-4-isopropoxyphenyl)-1,2,4-oxadiazol-5-yl)phenylamino)cyclobutanecarboxylic acid

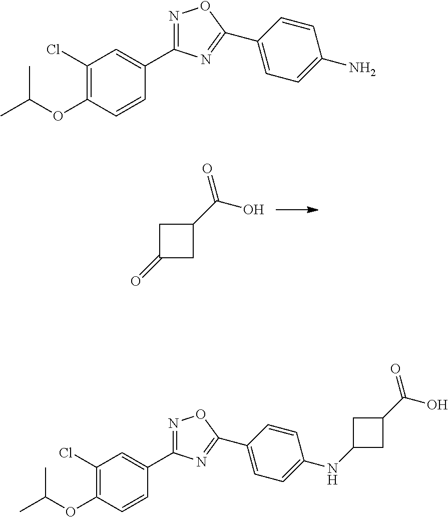

4-(3-(3-Chloro-4-isopropoxyphenyl)-1,2,4-oxadiazol-5-yl)aniline (250 mg, 0.531 mmol) in methanol (1478 μL) at RT was added 3-oxocyclobutanecarboxylic acid (60.5 mg, 0.531 mmol) followed by acetic acid (814 μL, 14.22 mmol). The reaction mixture was stirred at RT for about 5 min before the addition of sodium cyanoborohydride (16.67 mg, 0.265 mmol). The reaction mixture was stirred overnight at RT. The solvent was removed and the crude material purified by FCC (50% ethyl acetate/heptane) to afford 3-(4-(3-(3-chloro-4-isopropoxyphenyl)-1,2,4-oxadiazol-5-yl)phenylamino)cyclobutanecarboxylic acid (139 mg, 0.302 mmol, 56.9% yield) as a white solid. LC/MS (Table A, Method b) 2.89 min, (M+H)⁺ 428.20.

### Preparation #37

### 3-Hydroxy-cyclobutanecarboxylic acid tert-butyl ester

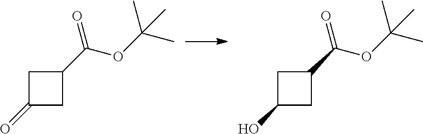

A solution of tert-butyl 3-oxocyclobutanecarboxylate (prepared according to R. P. Lemieux, J. Org. Chem. (1993), Vol. 58, No. 1, pp. 100-110) (10.5 g, 61.7 mmol) in EtOH (110 mL) was treated with sodium borohydride (2.173 mL, 61.7 mmol) portionwise at RT (cold water bath used to maintain reaction temperature below about 30° C.) and the reaction was stirred at room temperature for about 2 h. The reaction was diluted with saturated NaCl solution (300 mL) and extracted with EtOAc (300 mL). The EtOAc layer was washed with saturated salt solution (3×200 mL), dried over sodium sulfate, filtered and concentrated. The crude product was further purified by distillation, taking the fraction that boils at about 88-90° C. at 3 Torr to yield 3-hydroxy-cyclobutanecarboxylic acid tert-butyl ester (7.37 g, 70%) as colorless oil.

LC/MS (Table 1, Method a), Rₜ=no peak, no parent ion; ¹H NMR (400 MHz, DMSO-d6) δ ppm 5.12 (d, J=6.9, 1H), 3.97-3.88 (m, 1H), 2.46-2.29 (m, 3H), 1.94-1.86 (m, 2H), 1.39 (s, 9H).

### Preparation #38

### 4-Hydroxy-cyclohexanecarboxylic acid tert-butyl ester

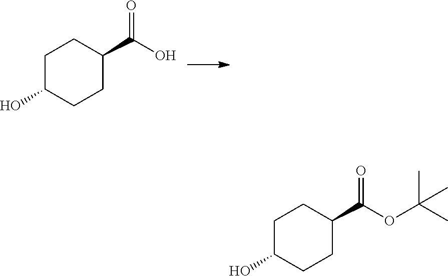

To a suspension of trans-4-hydroxycyclohexanecarboxylic acid (3.25 g, 22.54 mmol) in dichloroethane (150 mL) was added tert-butyl 2,2,2-trichloroacetimidate (16.15 mL, 90 mmol) and mixture was heated at about 80° C. under nitrogen for about 4 days. The reaction was cooled to RT, filtered and the white solid was rinsed with 10 mL methylene chloride. The organic layer was washed with saturated sodium bicarbonate solution (100 mL), filtered and concentrated. The residue was triturated twice with 10 mL of 80:20/heptane: ethyl acetate and filtered. The filtrate was injected onto a silica gel column and eluted with a gradient from 20% to 60% ethyl acetate in heptane over about 40 min. The combined product fractions were concentrated to an oil that solidifies on drying to constant weight under vacuum to yield 4-hydroxy-cyclohexanecarboxylic acid tert-butyl ester (1.95 g, 45%) as a white solid.

LC/MS (Table 1, Method f) Rₜ=no peak, no parent ion; ¹H NMR (400 MHz, DMSO-d6) δ ppm 4.52 (d, J=4.3, 1H), 3.38-3.29 (m, 1H), 2.10-2.02 (m, 1H), 1.82-1.79 (m, 4H), 1.38 (s, 9H), 1.34-1.24 (m, 2H), 1.18-1.08 (m, 2H).

### Preparation #39

### 3-Hydroxy-cyclopentanecarboxylic acid ethyl ester

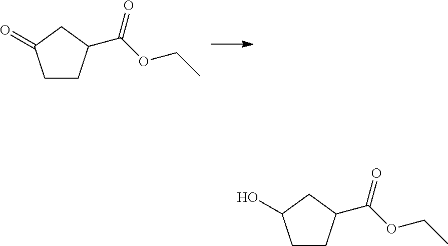

A solution of ethyl 3-oxocyclopentanecarboxylate (2.00 g, 12.81 mmol) in EtOH (20 mL) was treated with sodium borohydride (0.451 mL, 12.81 mmol) portionwise at RT (RT water bath to control exotherm) and the reaction was stirred overnight. The reaction was quenched with 2N HCl to about pH=2 and extracted with CH₂Cl₂, washed with water, dried over sodium sulfate, filtered and concentrated to oil. The crude product was further purified on silica gel using a gradient of 20-40% ethyl acetate in heptane. Pure product fractions and concentrate were combined to constant weight. NMR indicates an approximately 3:7 ratio of isomers. ¹H NMR (400 MHz, DMSO-d6) δ ppm 4.52-4.50 (m, 1H), 4.19-4.15 (m, 0.3H), 4.08-4.02 (m, 2.7H), 2.94-2.86 (m, 0.3H), 2.74-2.66 (m, 0.7H), 2.08-1.46 (m, 6H), 1.19-1.15 (m, 3H).

### Preparation #40

### (1R,3S)-3-(4-cyanophenylamino)cyclopentane carboxylic acid

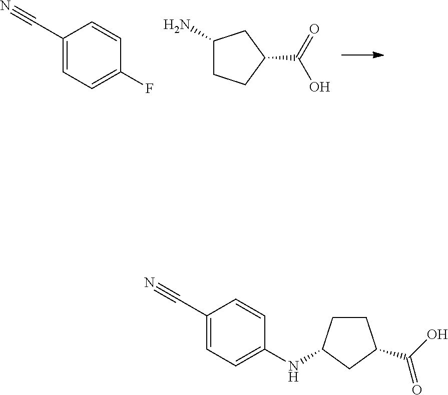

To a round bottom flask was added 4-fluorobenzonitrile (1.705 g, 14.08 mmol), (1R,3S)-3-aminocyclopentanecarboxylic acid (2 g, 15.49 mmol), potassium carbonate (4.28 g, 31 mmol), DMSO (45 mL) and water (1 mL). The mixture was heated at about 100° C. for about 16 h. The mixture was then cooled to ambient temperature and partitioned between water (250 mL) and EtOAc (250 mL). The aqueous layer was extracted with EtOAc (2×100 mL). The combined organic layers were dried over Na₂SO₄, filtered and concentrated to dryness to yield (1R,3S)-3-(4-cyanophenylamino)cyclopentane carboxylic acid (1.83 g, 7.87 mmol, 55.9%) as an off-white solid. LC/MS (Table 1, Method b) Rₜ=1.94 min, m/z 231 (M+H)+. ¹H NMR (400 MHz, DMSO-d₆) δ ppm 12.1 (s, 1H), 7.44-7.42 (d, 2H), 6.74-6.72 (d, 1H), 6.63-6.61 (d, 2H), 3.81-3.74 (m, 1H), 2.79-2.71 (m, 1H), 2.37-2.27 (m, 1H), 2.01-1.93 (m, 1H), 1.89-1.83 (m, 2H), 1.65-1.58 (m, 1H), 1.54-1.45 (m, 1H).

### Preparation #41

### Preparation of (1R,3S)-ethyl 3-(4-cyanophenylamino)cyclopentane carboxylate

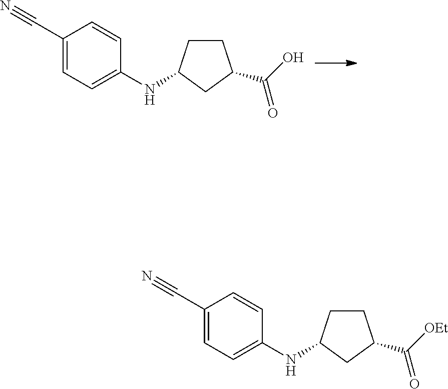

A solution of (1R,3S)-3-(4-cyanophenylamino)cyclopentane carboxylic acid (1.83 g, 7.87 mmol) in DMF (79 mL) was treated with DIEA (2.78 mL, 15.89 mmol) and HATU (3.63 g, 9.54 mmol) at RT. EtOH (0.696 mL, 11.92 mmol) was then added and the reaction was stirred for about 16 h. The reaction was concentrated under reduced pressure and the residue was partitioned between saturated sodium carbonate solution (100 mL) and EtOAc (2×100 mL). The organic layers were dried over Na₂SO₄, filtered and concentrated under reduced pressure. The residue was purified by silica gel chromatography (120 g, 0-60% EtOAc:Heptane) to afford (1R,3S)-ethyl 3-(4-cyanophenylamino)cyclopentane carboxylate (1.66 g, 6.43 mmol, 81%) as an oil. LC/MS (Table 1, Method b) Rₜ=2.38 min, m/z 259 (M+H)+. ¹H NMR (400 MHz, DMSO-d₆) δ ppm 7.44-7.42 (d, 2H), 6.74-6.72 (d, 1H), 6.63-6.61 (d, 2H), 4.08-4.03 (q, 2H), 3.82-3.77 (m, 1H), 2.87-2.79 (m, 1H), 2.35-2.28 (m, 1H), 2.03-1.94 (m, 1H), 1.90-1.85 (m, 2H), 1.63-1.59 (m, 1H), 1.53-1.47 (m, 1H), 1.18-1.14 (t, 3H).

### Preparation #42

### (1R,3S)-ethyl 3-(2-bromo-4-cyanophenylamino)cyclopentane carboxylate

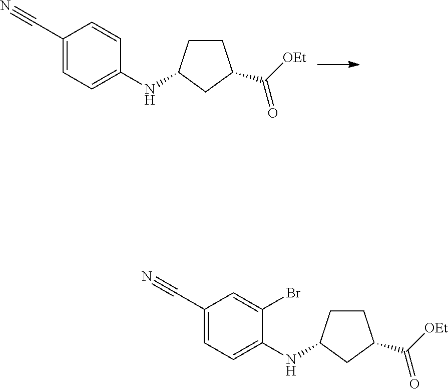

Sodium perborate monohydrate (0.353 g, 3.54 mmol) was added dropwise to a suspension of KBr (0.481 g, 4.04 mmol), (1R,3S)-ethyl 3-(4-cyanophenylamino)cyclopentane carboxylate (0.870 g, 3.37 mmol) and ammonium molybdate tetrahydrate (0.017 mL, 0.034 mmol) in acetic acid (11.2 mL). The reaction was stirred for about 16 h at RT and then concentrated acetic acid was removed under reduced pressure. Saturated NaCO₃ solution (50 mL) was added and the residue was extracted with EtOAc (100 mL). The organic layer was washed with a saturated Na₂CO₃ solution, dried over Na₂SO₄, filtered and concentrated under reduced pressure to yield (1R,3S)-ethyl 3-(2-bromo-4-cyanophenylamino)cyclopentane carboxylate (1.112 g, 3.30 mmol, 98%) as a tan oil. LC/MS (Table 1, Method b) Rₜ=2.64 min, m/z 337 (M+H)+. ¹H NMR (400 MHz, DMSO-d₆) δ ppm 7.89 (d, 1H), 7.61-7.58 (dd, 1H), 6.85-6.82 (d, 1H), 5.77-5.75 (d, 1H), 4.10-4.05 (q, 2H), 4.04-395 (m, 1H), 2.94-2.86 (m, 1H), 2.29-2.24 (m, 1H), 2.02-1.80 (m, 4H), 1.71-1.63 (m, 1H), 1.19-1.16 (t, 3H).

### Preparation #43

### Preparation of (1R,3S)-ethyl 3-(4-cyano-2-methylphenylamino)cyclopentane carboxylate

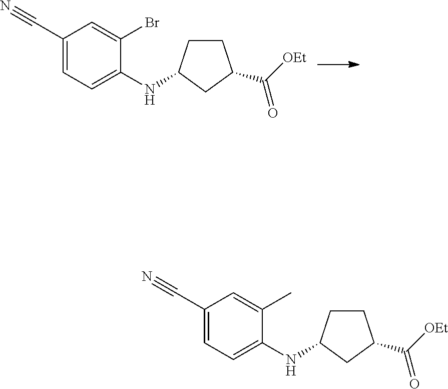

A solution of (1R,3S)-ethyl 3-(2-bromo-4-cyanophenylamino)cyclopentane carboxylate (1.12 g, 3.32 mmol), cesium carbonate (3.25 g, 9.96 mmol), trimethylboroxine (1.668 g, 6.64 mmol) and bis(triphenylphosphine)palladium(II) chloride (0.117 g, 0.166 mmol) in DME (16.6 mL) and water (5.5 mL) was purged with nitrogen for about 15 min. The reaction was stirred at about 90° C. and after about 4 h the reaction was cooled to ambient temperature. DME was removed under reduced pressure and the residue was partitioned between EtOAc (25 mL) and saturated NaCO₃ solution (25 mL). The aqueous layer was extracted again with EtOAc (50 mL) and the combined organic layers were dried over Na₂SO₄, filtered and concentrated under reduce pressure. The residue was purified by silica gel chromatography (120 g, 0-40% EtOAc:Heptane) to yield (1R,3S)-ethyl 3-(4-cyano-2-methylphenylamino)cyclopentane carboxylate (0.66 g, 2.42 mmol, 73%) as an oil. LC/MS (Table 1, Method b) Rₜ=2.61 min, m/z 273 (M+H)+. ¹H NMR (400 MHz, DMSO-d₆) δ ppm 7.43-7.40 (dd, 1H), 7.33 (s, 1H), 6.66-6.63 (d, 1H), 5.55-5.53 (d, 1H), 4.09-4.04 (q, 2H), 3.95-3.87 (m, 1H), 2.82-2.90 (m, 1H), 2.31-2.24 (m, 1H), 2.08 (s, 3H), 2.00-1.87 (m, 3H), 1.85-1.77 (m, 1H), 1.68-1.61 (m, 1H), 1.19-1.15 (t, 3H).

### Preparation #44

### Preparation of 4-(4-fluoropiperidin-1-yl)-3-(trifluoromethyl)benzonitrile

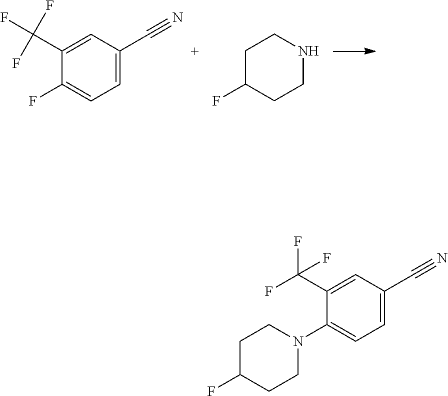

In a 200 mL round bottom flask, 4-fluoro-3-trifluoromethyl-benzonitrile (5.43 g, 28.7 mmol), 4-fluoropiperidine hydrochloride (4.407 g, 31.6 mmol), and potassium carbonate (9.92 g, 71.7 mmol) in DMF (50 mL) were added to give a tan suspension. The mixture was heated at about 90° C. for about 40 h. After cooling to ambient temperature, the reaction mixture was partitioned between water (75 mL) and ethyl acetate (75 mL). The organic layer was washed with water (50 mL) and brine (50 mL). The organic layer was dried over anhydrous magnesium sulfate and the solvent was removed in vacuo. The crude material was purified via gradient normal phase chromatography on silica gel (0% to 100% ethyl acetate in heptane over 6 column volumes) to afford 4-(4-fluoropiperidin-1-yl)-3-(trifluoromethyl)benzonitrile (4.87 g, 17.9 mmol, 62% yield). LC/MS R_{f}=7.51 min.; MS m/z: (M+H+AcOH)⁺. (Table 1, Method n). ¹H NMR (400 MHz, DMSO-d₆) ppm 8.17 (d, J=1.98, 1H), 8.06 (dd, J=2.01, 8.51, 1H), 7.59 (d, J=8.53, 1H), 4.87 (tdd, J=3.26, 6.66, 48.35, 1H), 3.13 (t, J=10.01, 2H), 3.02-2.92 (m, 2H), 2.07-1.78 (m, 4H).

### Preparation #45

### Preparation of (Z)-4-(4-fluoropiperidin-1-yl)-N′-hydroxy-3-(trifluoromethyl)benzimidamide

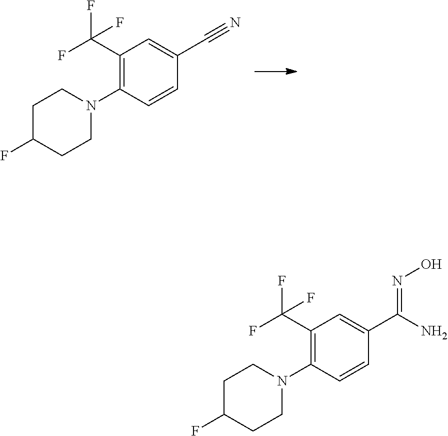

4-(4-Fluoro-piperidin-1-yl)-N-hydroxy-3-trifluoromethyl-benzamidine Prepared using General procedure C. LC/MS R_{f}=5.53 min.; MS m/z: 306.17 (M+H)⁺. (Table 1, Method n)

¹H NMR (400 MHz, DMSO-d₆) δ 9.74 (s, 1H), 7.95 (d, J=1.8, 1H), 7.91 (dd, J=1.8, 8.4, 1H), 7.53 (d, J=8.4, 1H), 5.93 (s, 2H), 4.94-4.74 (m, 1H), 3.01 (t, J=9.2, 2H), 2.88-2.78 (m, 2H), 2.06-1.90 (m, 2H), 1.84 (dqd, J=3.7, 6.8, 13.5, 2H).

### Preparation #46

### Preparation of 4-(3-(4-(4-fluoropiperidin-1-yl)-3-(trifluoromethyl)phenyl)-1,2,4-oxadiazol-5-yl)benzonitrile

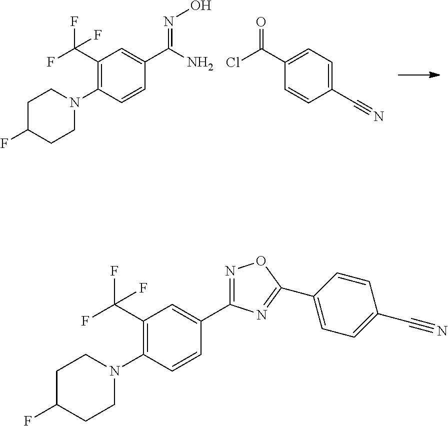

4-{3-[4-(4-Fluoro-piperidin-1-yl)-3-trifluoromethyl-phenyl-1,2,4]oxadiazol-5-yl}-benzonitrile Prepared using General procedure E. LC/MS R_{f}=2.26 min.; MS m/z: 417.18 (M+H)⁺ (Table 1, Method o) ¹H NMR (400 MHz, DMSO-d₆) δ 8.38 (d, J=8.3, 2H), 8.32 (dd, J=1.9, 8.4, 1H), 8.29 (d, J=1.9, 1H), 8.14 (d, J=8.3, 2H), 7.72 (d, J=8.4, 1H), 4.98-4.78 (m, 1H), 3.19-3.05 (m, 2H), 3.02-2.90 (m, 2H), 2.02 (dddd, J=3.6, 6.3, 11.5, 13.4, 2H), 1.94-1.81 (m, 2H).

### Preparation #47

### Reaction to produce 4-(3-(4-(4-fluoropiperidin-1-yl)-3-(trifluoromethyl)phenyl)-1,2,4-oxadiazol-5-yl)benzaldehyde

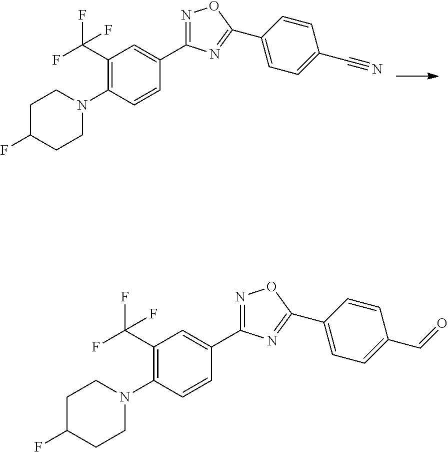

4-{3-[4-(4-Fluoro-piperidin-1-yl)-3-trifluoromethyl-phenyl-1,2,4]oxadiazol-5-yl}-benzaldehyde Prepared using General Procedure G. LC/MS R_{f}=4.20 min.; MS m/z: 421.15 (M+H)⁺ (Table 1, Method p) ¹H NMR (400 MHz, DMSO-d₆) δ 10.16 (s, 1H), 8.42 (d, J=8.2, 2H), 8.33 (dd, J=2.0, 8.4, 1H), 8.30 (d, J=2.0, 1H), 8.17 (d, J=8.6, 2H), 7.72 (d, J=8.4, 1H), 4.88 (dtt, J=3.3, 6.6, 48.4, 1H), 3.16-3.06 (m, 2H), 3.01-2.91 (m, 2H), 2.10-1.94 (m, 2H), 1.94-1.80 (m, 2H).

### Example #45

### Preparation of 1-(4-(3-(4-(4-fluoropiperidin-1-yl)-3-(trifluoromethyl)phenyl)-1,2,4-oxadiazol-5-yl)benzyl)azetidine-3-carboxylic acid, Ammonium Acetate salt

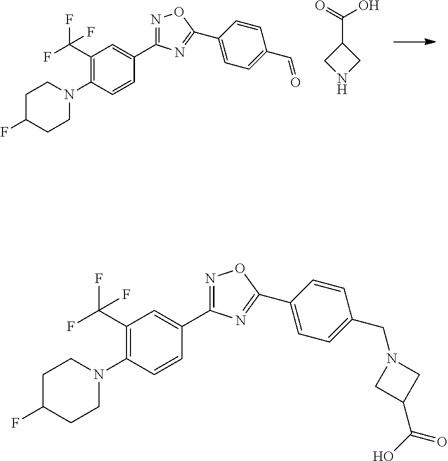

1-(4-{3-[4-(4-Fluoro-piperidin-1-yl)-3-trifluoromethyl-phenyl]-[1,2,4]oxadiazol-5-yl}-benzyl)-azetidine-3-carboxylic acid Prepared using General Procedure H. LC/MS Rₜ=2.07 min.; MS m/z: 505.19 (M+H)⁺, (Table 1, Method g). ¹H NMR (400 MHz, DMSO-d₆). δ ppm 8.33-8.26 (m, 2H), 8.15 (d, J=8.35 Hz, 2H), 7.71 (d, J=8.43 Hz, 1H), 7.55 (d, J=8.43 Hz, 2H), 4.88 (dddd, J=17.52, 10.37, 6.67, 3.53 Hz, 1H), 3.68 (s, 2H), 3.47-3.16 (m, 12H), 3.16-3.06 (m, 2H), 2.98-2.90 (m, 2H), 2.09-1.81 (m, 1H), 1.91 (s, 3H).

### Example #46

### Preparation of 5-((4-(3-(4-(4-fluoropiperidin-1-yl)-3-(trifluoromethyl)phenyl)-1,2,4-oxadiazol-5-yl)benzylamino)methyl)isoxazol-3-ol

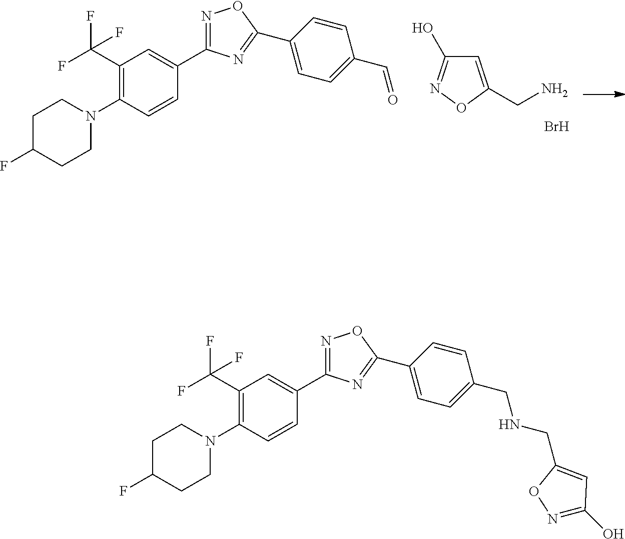

Prepared using General Procedure H. LC/MS Rₜ=2.69 min.; MS m/z: 516.18 (M−H)⁻, Table 1, method g. 1H NMR (400 MHz, CDCl3). δ ppm 8.50-8.42 (m, 1H), 8.28 (d, J=1.11 Hz, 1H), 8.19 (d, J=8.14 Hz, 2H), 7.55 (d, J=8.25 Hz, 3H), 7.44 (d, J=8.32 Hz, 1H), 5.89 (s, 1H), 4.96-4.74 (m, 1H), 3.94 (s, 2H), 3.86 (s, 2H), 3.25-3.09 (m, 2H), 3.00-2.87 (m, 2H), 2.05 (m, 4H).

### Example #47

### Preparation of 2-((4-(3-(4-isopropoxy-3-(trifluoromethyl)phenyl)-1,2,4-oxadiazol-5-yl)phenoxy)methyl)morpholine

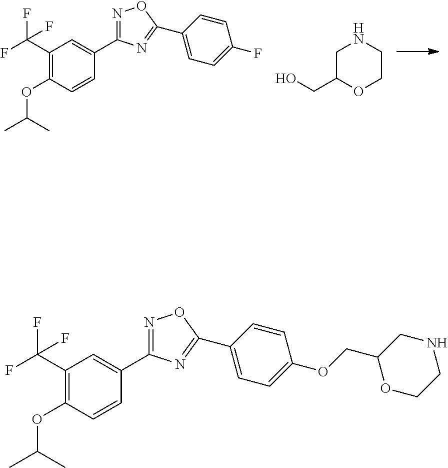

In a 25 mL round bottom flask, 2-hydroxymethylmorpholine (0.096 g, 0.819 mmol) (TYGER) in THF (10 mL) was added to give a colorless solution. Sodium hydride (0.020 g, 0.819 mmol) was added and the mixture was stirred until hydrogen evolution ceased. 5-(4-fluorophenyl)-3-(4-isopropoxy-3-(trifluoromethyl)phenyl)-1,2,4-oxadiazole (0.300 g, 0.819 mmol) was added and the mixture was stirred at ambient temperature under a nitrogen atmosphere then heated at reflux for about 4 days. The solvent was removed in vacuo. The crude material was purified by gradient normal phase chromatography using 0-10% methanol in dichloromethane as an eluent to afford 2-((4-(3-(4-isopropoxy-3-(trifluoromethyl)phenyl)-1,2,4-oxadiazol-5-yl)phenoxy)methyl)morpholine (0.032 g, 0.068 mmol, 8.4% yield). LC/MS Rₜ=2.29 min.; MS m/z: 464.15 (M+H)⁺. (Table 1, Method j). ¹H NMR (400 MHz, DMSO) δ ppm 8.27 (dd, J=8.78, 2.11 Hz, 1H), 8.19 (d, J=2.06 Hz, 1H), 8.11 (d, J=8.95 Hz, 2H), 7.51 (d, J=8.93 Hz, 1H), 7.18 (d, J=8.98 Hz, 2H), 4.90 (td, J=12.15, 6.07 Hz, 1H), 4.09-4.02 (m, 2H), 3.76-3.67 (m, 2H), 3.49-3.42 (m, 1H), 3.15 (d, J=5.20 Hz, 1H), 2.86 (dd, J=12.11, 2.23 Hz, 1H), 2.71-2.59 (m, 2H), 2.55-2.45 (m, 1H), 1.33 (d, J=6.02 Hz, 6H).

### Preparation #48 and 49

### Preparation of (R)- and (S)-4-(tetrahydrofuran-3-yloxy)-3-(trifluoromethyl)benzonitrile according to Scheme AA

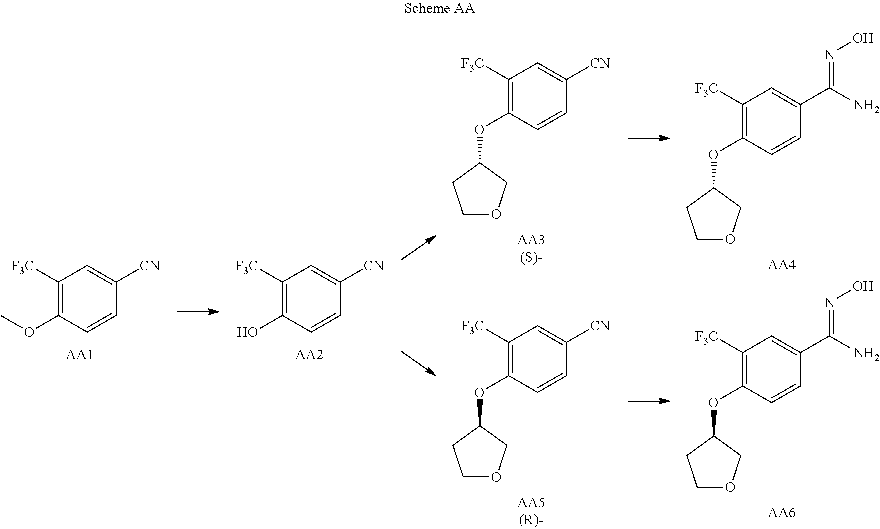

A mixture of 4-methoxy-3-(trifluoromethyl)benzonitrile (AA1, 24.88 g, 0.124 mol) and pyridine hydrochloride (29.04 g, 0.251 mol) was stirred and heated at about 200° C. for about 40 min. The mixture was cooled to ambient temperature before the addition of water and the resulting precipitate was filtered and dried to give 21.1 g of 4-hydroxy-3-(trifluoromethyl)benzonitrile (AA2) which was used without further purification. Yield: 91%.

To a stirred mixture of 4-hydroxy-3-(trifluoromethyl)benzonitrile (AA2, 9.9 g, 53 mmol) and PPh₃ (23.6 g, 90 mmol) in THF (250 mL) at about 0° C., DIAD (17.7 mL, 90 mmol) was added The mixture was stirred for about 10 min. at about 0° C. before (R)-(−)-3-hydroxytetrahydrofuran (5 g, 56.7 mmol) in THF (20 mL) was added. The mixture was stirred at ambient temperature overnight under an atmosphere of nitrogen. The solvent was removed in vacuo and the residue was purified by flash column chromatography (EA/petroleum ether=10-20%) to give (R)-4-(tetrahydrofuran-3-yloxy)-3-(trifluoromethyl)benzonitrile (AA5, 8.8 g, 65% yield) which was used without further purification.

19 mL (96 mmol) of diisopropyl azodicarboxylate was added to the mixture of 4-hydroxy-3-(trifluoromethyl)benzonitrile (AA2, 11 g, 59 mmol) and triphenylphosphine (25.3 g, 96 mmol) in THF (250 mL) at about 0° C. The mixture was stirred for about 10 min. at about 0° C. (S)-(+)-3-hydroxytetrahydrofuran (5 g, 56.7 mmol) in THF (20 mL) was added. The mixture was stirred at RT overnight under nitrogen. The solvent was removed in vacuo and the residue was purified by flash column chromatography (ethyl acetate/petroleum ether=10-20%) to afford (S)-4-(tetrahydrofuran-3-yloxy)-3-(trifluoromethyl)benzonitrile. (AA3, 9.2 g, yield: 63%) which was used without further purification.

A solution of (R)-4-(tetrahydrofuran-3-yloxy)-3-(trifluoromethyl)benzonitrile (AA5, 8.6 g, 33 mmol) in ethanol (80 mL) was treaded with 50% aqueous hydroxylamine (8.1 mL). The mixture was stirred and heated at about 60° C. for about 18 h. The solvents were removed in vacuo and the residue was purified by flash column chromatography (methanol/chloroform=5-15%) to give (R)—N′-hydroxy-4-(tetrahydrofuran-3-yloxy)-3-(trifluoromethyl)benzimidamide (AA6, 5.4 g, yield: 56%) LC/MS (Table 1, Method c) Rₜ=2.48 min, m/z 290.0 (M+H)⁺; ¹H NMR (400 MHz, DMSO-d₆) δ 9.64 (s, 1H), 7.89 (m, 2H), 7.27 (d, 1H), 5.91 (s, 2H), 5.24 (m, 1H), 3.95 (dd, 1H), 3.79 (ddd, 3H), 2.24 (dtd, 1H), 1.98 (td, 1H).

A solution of (S)-4-(tetrahydrofuran-3-yloxy)-3-(trifluoromethyl)benzonitrile (AA3, 9 g, 35 mmol) in ethanol (80 mL) was treaded with 50% aqueous hydroxylamine (8.5 mL). The mixture was stirred and heated at about 60° C. overnight. The solvents were removed in vacuo and the residue was purified by flash column chromatography (methanol/chloroform=5-15%) to afford N-Hydroxy-4-[(S)-(tetrahydro-furan-3-yl)oxy]-3-trifluoromethyl-benzamidine. (AA4, 5.6 g, yield: 55%) which was used without further purification.

### Preparation #50

### (S)-5-(4-Fluorophenyl)-3-(4-(tetrahydrofuran-3-yloxy)-3-(trifluoromethyl)phenyl)-1,2,4-oxadiazole

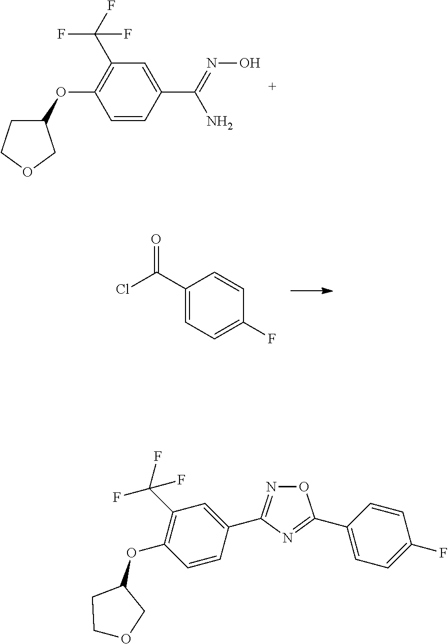

(S,Z)-M-hydroxy-4-(tetrahydrofuran-3-yloxy)-3-(trifluoromethyl)benzimidamide (500 mg, 1.723 mmol), 4-fluorobenzoyl chloride (0.207 mL, 1.723 mmol) and pyridine (5 mL) are loaded into a microwave vial equipped with a stir bar. The vessel is sealed and the reaction heated to about 160° C. with cooling for about 25 min. The mixture was diluted with DMSO (2 mL) and analyzed by LC/MS. MeCN (6 mL) was added and the mixture subjected to purification by molecular ion directed LC/MS to afford (S)-5-(4 fluorophenyl)-3-(4-(tetrahydrofuran-3-yloxy)-3-(trifluoromethyl)phenyl)-1,2,4-oxadiazole (413 mg, 1.047 mmol, 60.8% yield) as an off-white solid. LC/MS (Table 1, Method g) Rₜ=3.15 min.; MS m/z: did not ionize. ¹H NMR (400 MHz, DMSO) d ppm 8.42-8.14 (m, 4H), 7.60-7.43 (m, 3H), 5.35 (t, J=5.02 Hz, 1H), 3.98 (dd, J=10.49, 4.40 Hz, 1H), 3.91-3.76 (m, 3H), 2.30 (dd, J=13.95, 5.75 Hz, 1H), 2.11-1.96 (m, 1H).

### Example #48

### (1R,3S)-3-(4-(3-(4-((S)-Tetrahydrofuran-3-yloxy)-3-(trifluoromethyl)phenyl)-1,2,4-oxadiazol-5-yl)phenylamino)cyclopentanecarboxylic acid

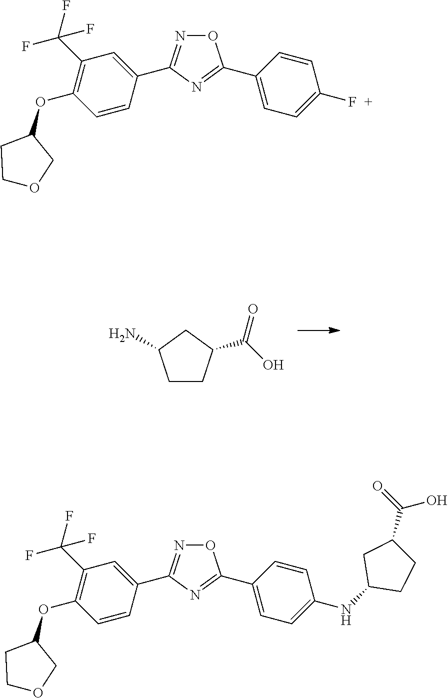

(S)-5-(4-fluorophenyl)-3-(4-(tetrahydrofuran-3-yloxy)-3-(trifluoromethyl)phenyl)-1,2,4-oxadiazole (350 mg, 0.888 mmol), (1R,3S)-3-aminocyclopentanecarboxylic acid (115 mg, 0.888 mmol), potassium carbonate (270 mg, 1.953 mmol) and DMF (2 mL) was heated with cooling at about 160° C. on the Biotage microwave for about 30 min. The mixture was diluted with DMSO (6 mL) and MeCN (8 mL), filtered and divided into 8 aliquots for purification by molecular ion directed LC/MS. The fractions were combined and evaporated to afford a pale brown solid that was dried in vacuo at about 60° C. for about 3 h to afford (1R,3S)-3-(4-(3-(4-((S)-tetrahydrofuran-3-yloxy)-3-(trifluoromethyl)phenyl)-1,2,4-oxadiazol-5-yl)phenylamino)cyclopentanecarboxylic acid (43 mg, 0.083 mmol, 9.33% yield) as a pale brown solid. NMR showed the presence of ammonium acetate and so the compound was dried in vacuo at about 100° C. for about 3 h to afford (1R,3S)-3-(4-(3-(4-((S)-tetrahydrofuran-3-yloxy)-3-(trifluoromethyl)phenyl)-1,2,4-oxadiazol-5-yl)phenylamino)cyclopentanecarboxylic acid (43 mg, 0.083 mmol, 9.33% yield) as an off-white solid. LC/MS (Table 1, Method g) Rₜ=2.61 min.; MS m/z: 504.2 (M+H)⁺. 1H NMR (400 MHz, DMSO) d ppm 7.48 (d, J=8.85 Hz, 1H), 7.03-6.89 (m, 1H), 6.72 (d, J=8.94 Hz, 2H), 7.87 (d, J=8.88 Hz, 2H), 8.20 (d, J=2.03 Hz, 1H), 8.27 (dd, J=8.73, 2.06 Hz, 1H), 5.37-5.27 (m, 1H), 3.98 (dd, J=10.41, 4.47 Hz, 1H), 3.90-3.74 (m, 4H), 2.75 (t, J=8.31 Hz, 1H), 2.30 (td, J=11.68, 6.98 Hz, 2H), 2.10-1.92 (m, 2H), 1.92-1.81 (m, 2H), 1.68 (dd, J=7.98, 4.74 Hz, 1H), 1.55 (dd, J=12.18, 6.65 Hz, 1H).

### Example #49

### (1R,3S)-3-(4-(3-(4-(4-Fluoropiperidin-1-yl)-3-(trifluoromethyl)phenyl)-1,2,4-oxadiazol-5-yl)phenylamino)cyclopentanecarboxylic acid

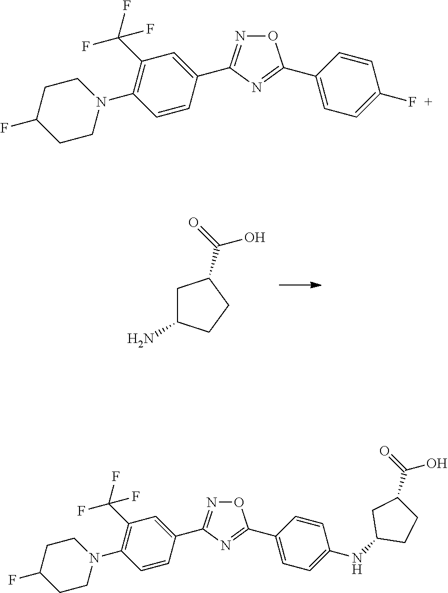

5-(4-Fluorophenyl)-3-(4-(4-fluoropiperidin-1-yl)-3-(trifluoromethyl)phenyl)-1,2,4-oxadiazole (420 mg, 1.026 mmol), (1R,3S)-3-aminocyclopentanecarboxylic acid (146 mg, 1.129 mmol), potassium carbonate (312 mg, 2.257 mmol) and DMF (2 mL) was heated with cooling at about 160° C. on the Biotage microwave for about 30 min. The mixture was diluted with DMSO (6 mL) and MeCN (8 mL), filtered and divided into 8 aliquots for purification by molecular ion directed LC/MS. The fractions were combined and evaporated to afford a pale brown solid that was dried in vacuo at about 60° C. for about 3 h to afford (1R,3S)-3-(4-(3-(4-(4-fluoropiperidin-1-yl)-3-(trifluoromethyl)phenyl)-1,2,4-oxadiazol-5-yl)phenylamino)cyclopentanecarboxylic acid (122 mg, 0.235 mmol, 22.93% yield) as an off-white solid. LC/MS (Table 1, Method f) Rₜ=3.05 min.; MS m/z: 517.17 (M−H)⁻. ¹H NMR (400 MHz, DMSO) δ ppm 6.83 (d, J=6.67 Hz, 1H), 6.72 (d, J=8.92 Hz, 2H), 8.28-8.21 (m, 2H), 7.87 (d, J=8.86 Hz, 2H), 7.67 (d, J=8.40 Hz, 1H), 12.16-12.05 (m, 1H), 4.97-4.75 (m, 1H), 3.84 (d, J=6.98 Hz, 1H), 3.08 (t, J=9.04 Hz, 2H), 1.65 (d, J=12.27 Hz, 1H), 1.52 (s, 1H), 2.31 (s, 1H), 2.11-1.92 (m, 3H), 1.92-1.79 (m, 4H), 2.97-2.85 (m, 2H), 2.83-2.69 (m, 1H).

### Example #50

### (1R,3S)-3-(4-(3-(4-(4,4-Difluoropiperidin-1-yl)-3-(trifluoromethyl)phenyl)-1,2,4-oxadiazol-5-yl)phenylamino)cyclopentanecarboxylic acid acetate salt

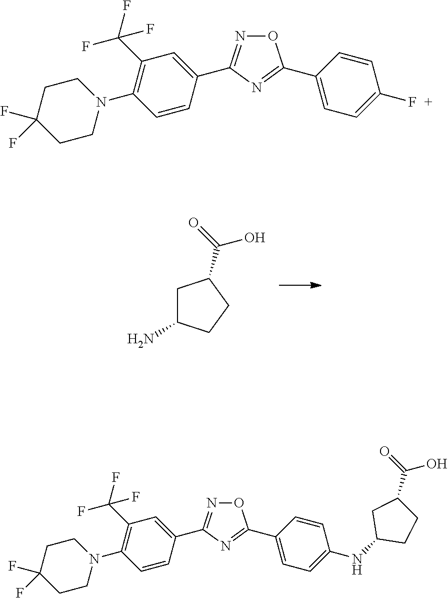

3-(4-(4,4-Difluoropiperidin-1-yl)-3-(trifluoromethyl)phenyl)-5-(4-fluorophenyl)-1,2,4-oxadiazole (503 mg, 1.177 mmol), (1R,3S)-3-aminocyclopentanecarboxylic acid (167 mg, 1.295 mmol), potassium carbonate (358 mg, 2.59 mmol) and DMF (2 mL) was heated with cooling at about 160° C. on the Biotage microwave for about 30 min. The mixture was diluted with DMSO (6 mL) and MeCN (8 mL), filtered and divided into 8 aliquots for purification by molecular ion directed LC/MS. The fractions were combined and evaporated to afford a pale brown solid that was dried in vacuo at about 60° C. for about 3 h to afford (1R,3S)-3-(4-(3-(4-(4,4-difluoropiperidin-1-yl)-3-(trifluoromethyl)phenyl)-1,2,4-oxadiazol-5-yl)phenylamino)cyclopentanecarboxylic acid (243 mg, 0.453 mmol, 38.5% yield) as an off-white solid as the acetate salt. LC/MS (Table 1, Method g) Rₜ=3.06 min.; MS m/z: 535.2 (M−H)⁻.

1H NMR (400 MHz, DMSO) δ ppm 7.73 (d, J=8.23 Hz, 1H), 7.86 (d, J=8.85 Hz, 2H), 8.29-8.21 (m, 2H), 2.81-2.68 (m, 1H), 2.30 (d, J=12.64 Hz, 1H), 2.10 (ddd, J=13.96, 10.71, 5.50 Hz, 4H), 1.98 (s, 1H), 1.92-1.81 (m, 2H), 1.66 (d, J=12.66 Hz, 1H), 1.52 (s, 1H), 3.09-3.04 (m, 4H), 3.87-3.79 (m, 1H), 2.52 (s, 3H).

### Example #51

### (1S,3S)-3-(4-(3-(4-(4-fluoropiperidin-1-yl)-3-(trifluoromethyl)phenyl)-1,2,4-oxadiazol-5-yl)phenylamino)cyclopentanecarboxylic acid acetate salt

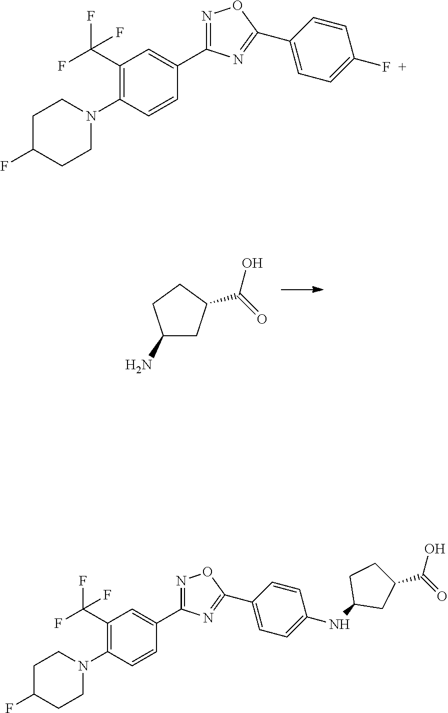

5-(4-Fluorophenyl)-3-(4-(4-fluoropiperidin-1-yl)-3-(trifluoromethyl)phenyl)-1,2,4-oxadiazole (576 mg, 1.408 mmol), (1S,3S)-3-aminocyclopentanecarboxylic acid (200 mg, 1.549 mmol, Afid Therapeutics), potassium carbonate (428 mg, 3.10 mmol) and DMF (2 mL) was heated with cooling at about 160° C. on the Biotage microwave for about 30 min. The mixture was diluted with DMSO (6 mL) and MeCN (8 mL), filtered and divided into 8 aliquots for purification by molecular ion directed LC/MS. The fractions were combined and evaporated to afford a pale brown solid that was dried in vacuo at about 60° C. for about 3 h to afford (1S,3S)-3-(4-(3-(4-(4-fluoropiperidin-1-yl)-3-(trifluoromethyl)phenyl)-1,2,4-oxadiazol-5-yl)phenylamino)cyclopentanecarboxylic acid (47 mg, 0.082 mmol, 5.80% yield) as a pale brown solid as the acetate salt. LC/MS (Table 1, Method g) Rₜ=3.03 min.; MS m/z: 517.2 (M−H)⁻. 1H NMR (400 MHz, DMSO) δ ppm 8.23 (td, J=5.39, 1.81 Hz, 2H), 7.65 (d, J=8.39 Hz, 1H), 6.69 (dd, J=9.02, 2.47 Hz, 2H), 6.79 (d, J=6.44 Hz, 1H), 7.86 (d, J=8.88 Hz, 2H), 4.97-4.87 (m, 1H), 4.83-4.74 (m, 1H), 3.96-3.79 (m, 2H), 3.07 (s, 2H), 2.97-2.85 (m, 2H), 2.85-2.72 (m, 1H), 2.20-1.43 (m, 13H).

### Example #52

### (1R,3R)-3-(4-(3-(4-(4-fluoropiperidin-1-yl)-3-(trifluoromethyl)phenyl)-1,2,4-oxadiazol-5-yl)phenylamino)cyclopentanecarboxylic acid acetate salt

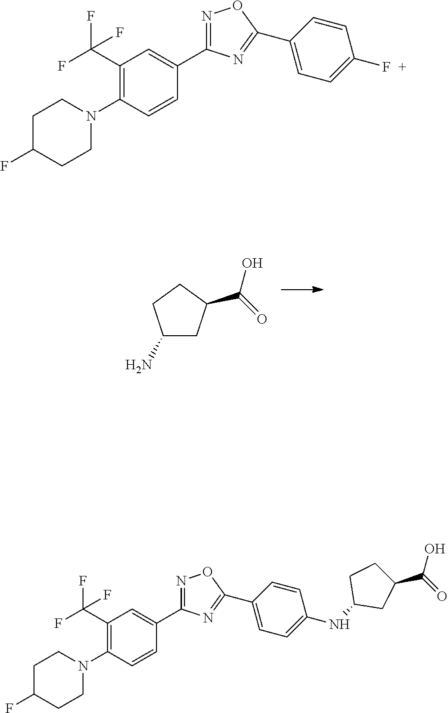

5-(4-Fluorophenyl)-3-(4-(4-fluoropiperidin-1-yl)-3-(trifluoromethyl)phenyl)-1,2,4-oxadiazole (576 mg, 1.408 mmol), (1R,3R)-3-aminocyclopentanecarboxylic acid (200 mg, 1.549 mmol, Afid Therapeutics), potassium carbonate (428 mg, 3.10 mmol) and DMF (3 mL) was heated with cooling at about 160° C. on the Biotage microwave for about 30 min. The mixture was diluted with DMSO (6 mL) and MeCN (8 mL), filtered and divided into 8 aliquots for purification by molecular ion directed LC/MS. The fractions were combined and evaporated to afford a pale brown solid that was dried in vacuo at about 60° C. for about 3 hours to afford (1R,3R)-3-(4-(3-(4-(4-fluoropiperidin-1-yl)-3-(trifluoromethyl)phenyl)-1,2,4-oxadiazol-5-yl)phenylamino)cyclopentanecarboxylic acid (43 mg, 0.075 mmol, 5.30% yield) as an pale brown solid as the acetate salt. LC/MS (Table 1, Method g Rₜ=3.03 min.; MS m/z: 517.2 (M−H)⁻. 1H NMR (400 MHz, DMSO) δ ppm 8.27-8.18 (m, 2H), 7.85 (dd, J=8.88, 3.58 Hz, 2H), 7.65 (d, J=8.35 Hz, 1H), 6.79 (d, J=6.47 Hz, 1H), 6.70 (d, J=8.94 Hz, 2H), 4.91 (dd, J=6.64, 3.30 Hz, 1H), 4.82-4.73 (m, 1H), 3.87 (d, J=5.83 Hz, 1H), 3.06 (t, J=8.93 Hz, 2H), 2.96-2.76 (m, 3H), 2.28-1.39 (m, 13H).

### Example #53

### (1S,3R)-3-(4-(5-(3-cyano-4-(4-fluoropiperidin-1-yl)phenyl)-1,2,4-oxadiazol-3-yl)-2-(trifluoromethyl)phenylamino)cyclopentanecarboxylic acid acetate salt

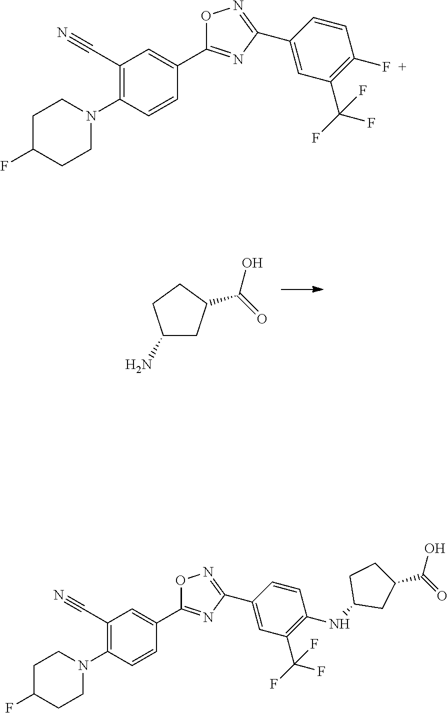

5-(3-(4-fluoro-3-(trifluoromethyl)phenyl)-1,2,4-oxadiazol-5-yl)-2-(4-fluoropiperidin-1-yl)benzonitrile (500 mg, 1.151 mmol), (1S,3R)-3-aminocyclopentanecarboxylic acid (164 mg, 1.266 mmol), potassium carbonate (350 mg, 2.53 mmol) and DMF (3 mL) was heated with cooling at about 160° C. on the Biotage microwave for about 30 min. The mixture was diluted with DMSO (12 mL) and ACN (9 mL), filtered and divided in 8 aliquots for purification by molecular ion directed LC/MS. The fractions were combined and evaporated to afford a pale brown solid that was dried in vacuo at about 60° C. for about 7 h to afford (1S,3R)-3-(4-(5-(3-cyano-4-(4-fluoropiperidin-1-yl)phenyl)-1,2,4-oxadiazol-3-yl)-2-(trifluoromethyl)phenylamino)cyclopentanecarboxylic acid (153 mg, 0.267 mmol, 23.23% yield) as a pale brown solid as the acetate salt.

LC/MS (Table 1, Method g) Rₜ=2.93 min.; MS m/z: 542.1 (M−H)⁻.

δ H (400 MHz, DMSO) 8.32 (1H, s), 8.19 (1H, d, J 8.9), 8.01 (2H, d, J 7.8), 7.31 (1H, d, J 8.9), 7.03 (1H, d, J 9.2), 5.96 (1H, s), 5.05-4.79 (1H, m), 4.12-3.97 (1H, m), 3.44 (4H, d, J 29.9), 2.84 (1H, s), 1.97 (11H, d, J 73.3).

### Example #54

### (1S,3R)-3-(4-(5-(3-cyano-4-(4-fluoropiperidin-1-yl)phenyl)-1,2,4-oxadiazol-3-yl)phenylamino)cyclopentanecarboxylic acid acetate salt

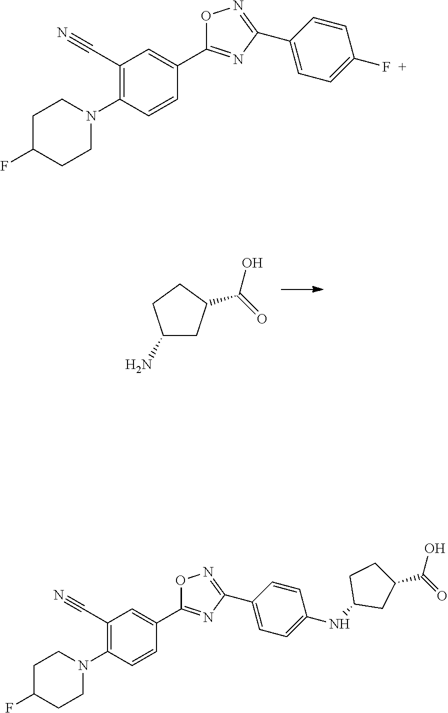

5-(3-(4-Fluorophenyl)-1,2,4-oxadiazol-5-yl)-2-(4-fluoropiperidin-1-yl)benzonitrile (500 mg, 1.365 mmol), (1S,3R)-3-aminocyclopentanecarboxylic acid (194 mg, 1.501 mmol), potassium carbonate (415 mg, 3.00 mmol) and DMF (3 mL) was heated with cooling at about 160° C. on the Biotage microwave for about 30 min. The mixture was diluted with DMSO (12 mL) and MeCN (9 mL), filtered and divided into 12 aliquots for purification by molecular ion directed LC/MS. The fractions were combined and evaporated to afford a pale brown solid that was dried in vacuo at about 60° C. for about 7 h to afford (1S,3R)-3-(4-(5-(3-cyano-4-(4-fluoropiperidin-1-yl)phenyl)-1,2,4-oxadiazol-3-yl)phenylamino)cyclopentanecarboxylic acid (23 mg, 0.046 mmol, 3.40% yield) as a pale yellow solid as the acetate salt. LC/MS (Table 1, Method g) Rₜ=2.59 min.; MS m/z: 474.2 (M−H)⁻.

δ H (400 MHz, DMSO) 8.33 (1H, s), 8.24-8.17 (1H, m), 7.75 (2H, d, J 8.6), 7.34 (1H, d, J 8.9), 6.65 (2H, d, J 8.7), 5.03-4.83 (2H, m), 3.84-3.72 (2H, m), 3.55-3.32 (6H, m), 2.75-2.58 (2H, m), 1.83 (15H, s).

### Preparation #51 and 52

### 5-[3-(4-Fluoro-phenyl)-[1,2,4]oxadiazol-5-yl]-2-(4-fluoro-piperidin-1-yl)-benzonitrile and 5-[3-(4-Fluoro-3-trifluoromethyl-phenyl)-[1,2,4]oxadiazol-5-yl]-2-(4-fluoro-piperidin-1-yl)-benzonitrile according to Scheme BB

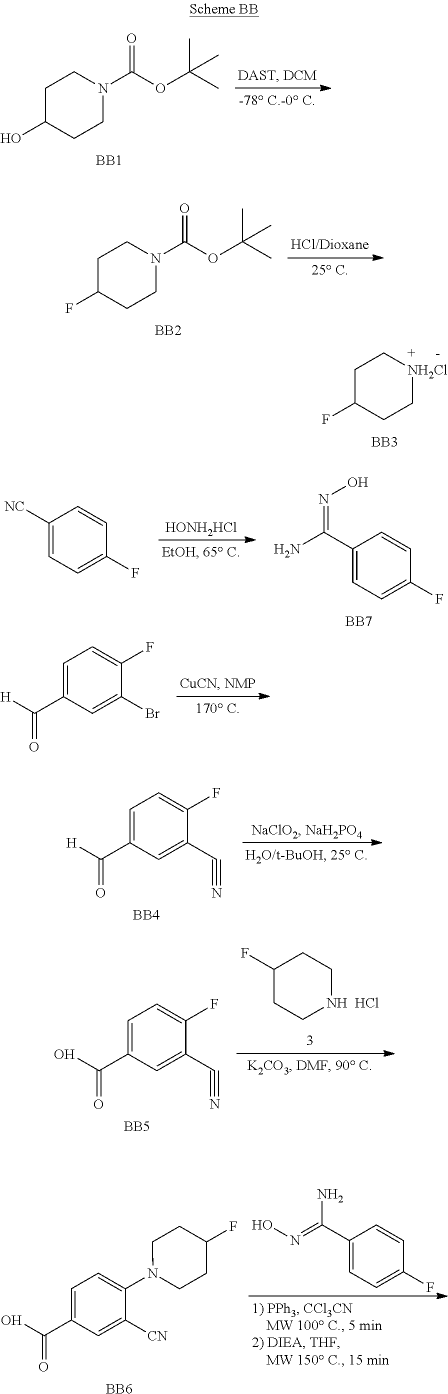

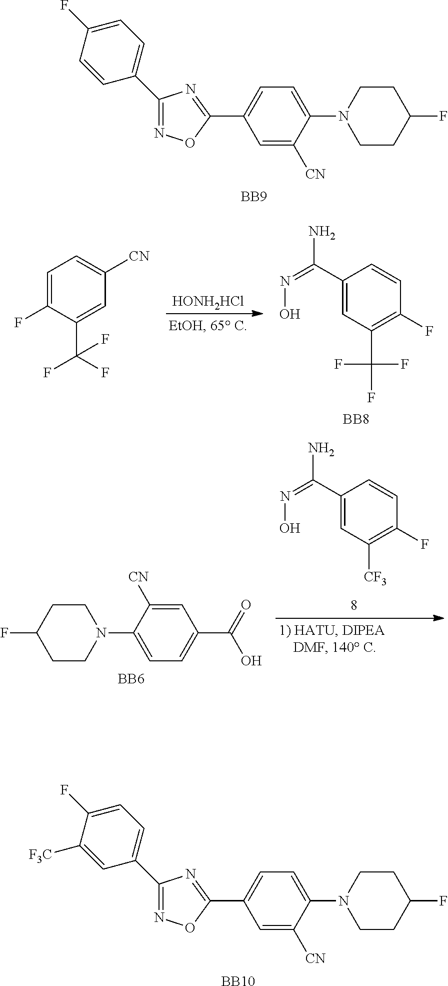

### Preparation of BB2: 4-Fluoro-piperidine-1-carboxylic Acid tert-butyl Ester

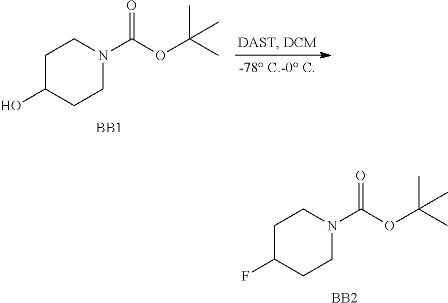

The solution of 4-hydroxy-piperidine-1-carboxylic acid tert-butyl ester (BB1, 20.0 g, 99.4 mmol) in DCM (400 mL) was cooled to about −78° C. and to this the DAST (31.65 mL) was slowly added dropwise over about 30 min. The mixture was stirred at about −78° C. for about 0.5 h and at about 0° C. for about 0.5 h. The analysis by TLC showed the formation of desired product which was then quenched by addition of saturated NaHCO₃ aqueous and chloroform. The organic layer was separated, dried over Na₂SO₄, and concentrated under reduced pressure. The crude product was further purified by silicon gel column chromatography to afford 4-Fluoro-piperidine-1-carboxylic acid tert-butyl ester as colorless oil (BB2, 8.0 g).

### Preparation of BB3: 4-fluoro-piperidine Hydrochloride

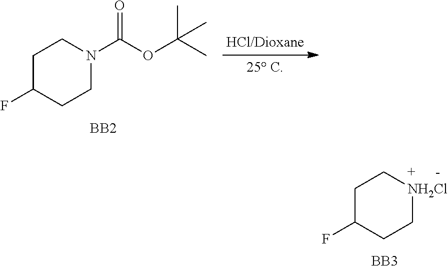

To a solution of 4-fluoro-piperidine-1-carboxylic acid tert-butyl ester 2 (BB2, 23.0 g, 113.2 mmol) in DCM (460 mL) the solution of HCl/1,4-dioxane (2 N, 200 mL) was slowly added dropwise at about 0° C. The mixture was stirred at about 25° C. overnight. The analysis by TLC indicated the completion of the reaction. The solution was concentrated under reduced pressure. The residue was washed with DCM, dried in vacuum to afford 4-fluoro-piperidine as white hydrochloride salt (BB3, 13.3 g).

### Preparation of BB4: 2-Fluoro-5-formyl-benzonitrile

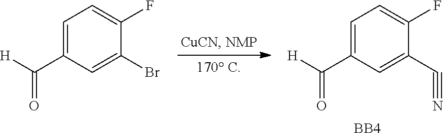

3-Bromo-4-fluoro-benzaldehyde (15.0 g, 74 mmol) and CuCN (7.6 g, 84.4 mmol) were mixed with NMP (27 mL) under vigorously stirring at about 25° C. The mixture was slowly heated over several hours to reach about 170° C. and stirred for about 24 h. The analysis by TLC showed the complete consumption of starting material and formation of new product. The temperature was cooled to about 80° C. and EtOAc (250 mL) and water (150 mL) was added. The organic layer was separated and the aqueous was extracted with EtOAc (150 mL×2). The combined organic layers were dried over NaSO₄ and concentrated. The residue was purified by silica gel column chromatography to afford 2-Fluoro-5-formyl-benzonitrile as a white solid (BB4, 9.0 g).

### Preparation of BB5: 3-Cyano-4-fluoro-benzoic Acid

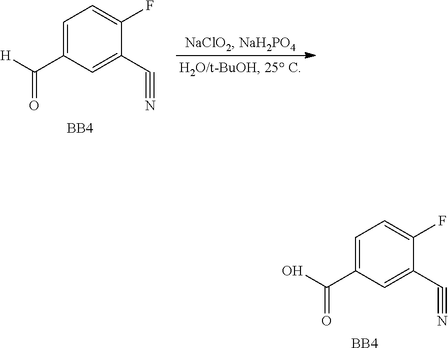

To a solution of the 2-fluoro-5-formyl-benzonitrile BB4 in mixed solvent of H₂O/t-BuOH (48 mL/201 mL), the NaClO₂ (28.2 g, 223.5 mmol) and NaH₂PO₄ (22.2 g, 142.6 mmol) was added. The mixture was stirred vigorously at about 25° C. and stood overnight. The analysis by TLC indicated the complete consumption of the starting material. The solution was adjusted to pH=3.5 by addition of 1 N aqueous HCl and extracted with DCM/1-Propanol (10:1, 200 mL×3). The combined organic layer was dried over Na₂SO₄ and concentrated in vacuum. The residue was redissolved in aqueous NaHCO₃ and extracted with DCM. The aqueous layer was acidified; the white precipitate was extracted with DCM, dried over Na₂SO₄ and concentrated under reduced pressure to afford 3-Cyano-4-fluoro-benzoic acid as white solid (BB5, 9.5 g).

### Preparation of BB8: 4-Fluoro-N-hydroxy-benzamidine

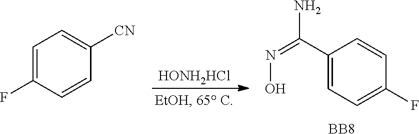

To a solution of the 4-fluoro-benzonitrile (10.0 g, 52.9 mmol) in ethanol (200 mL), the solution of NH₂OH.HCl (4.0 g, 58.2 mmol) in H₂O (4 mL) was added dropwise. The mixture was stirred vigorously at about 65° C. and stood overnight. The analysis by TLC indicated the complete consumption of starting material. The solution was concentrated to remove the solvent. The residue was dissolved in DCM and washed with water, brine, dried over Na₂SO₄ and concentrated in vacuum to afford 4-Fluoro-N-hydroxy-benzamidine as pale-yellow solid (BB8).

### Preparation of BB6: 3-Cyano-4-(4-fluoro-piperidin-1-yl)-benzoic acid

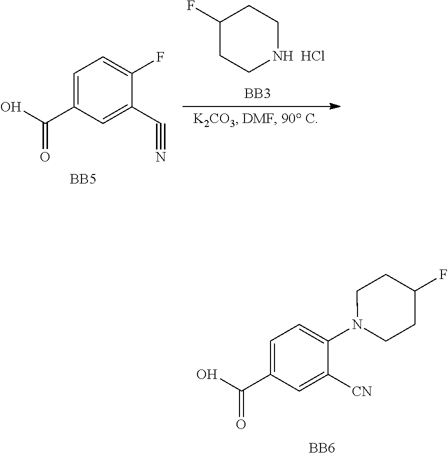

To a solution of the 3-cyano-4-fluoro-benzoic acid BB5 (14.3 g, 86.9 mmol) and K₂CO₃ (36.3 g, 26.1 mmol) in DMF (150 mL) the 4-fluoro-piperidinium hydrochloride BB3 (13.3 g, 95.6 mmol) was added neat at about 25° C. The mixture was heated to about 90° C. and stirred overnight. After about 24 h, the analysis by HPLC indicated there still remained starting material. The solution was stirred for another 24 h. The solution was quenched by addition of EtOAc and (300 mL) 1 N HCl. The organic layer was washed with brine, dried over Na₂SO₄, concentrated under reduced pressure. The residue was further purified by silica gel column chromatograph to afford 3-Cyano-4-(4-fluoro-piperidin-1-yl)-benzoic acid as white solid (BB6).

### Preparation of 5-[3-(4-Fluoro-phenyl)-[1,2,4]oxadiazol-5-yl]-2-(4-fluoro-piperidin-1-yl)-benzonitrile

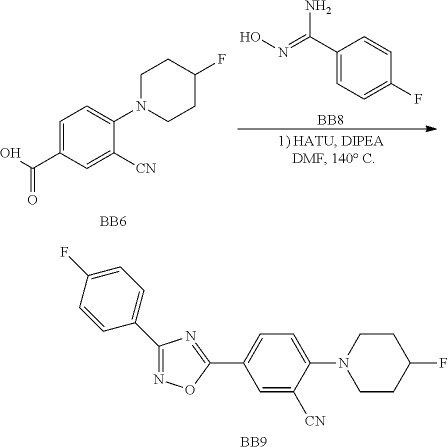

A solution of the 3-cyano-4-(4-fluoro-piperidin-1-yl)-benzoic acid BB6 (0.5 g×18, 2 mmol) and CCl₃CN (0.44 g×18, 3 mmol) in dry THF (15 mL) was prepared and to this the Ph₃P (1.58 g*18, 6 mmol) was added under protection of nitrogen at about 25° C. The mixture was reacted in microwave add to abbreviation list at about 100° C. for about 5 min, then a solution of 4-fluoro-N-hydroxy-benzamidine BB8 (0.34 g×18, 2.2 mmol) and DIEA (0.52 g×18, 4 mmol) was added. The mixture was reacted in MW at about 150° C. for about 15 min. The analysis by TLC showed the formation of desired product, which was purified by silica gel column chromatograph to afford 5-[3-(4-fluoro-phenyl)-[1,2,4]oxadiazol-5-yl]-2-(4-fluoro-piperidin-1-yl)-benzonitrile as white solid (BB9). 1H NMR (400 MHz, DMSO) δ ppm 8.31 (1H, m), 8.19-8.17 (1H, m), 8.08-8.05 (2H, m), 7.41-7.36 (2H, m), 7.32-7.30 (1H, m), 4.97-4.85 (1H, m), 3.50-3.35 (4H, m), 2.07-1.91 (2H, m) and 1.89-1.85 (2H, m).

### Preparation of BB7: 4-Fluoro-N-hydroxy-3-trifluoromethyl-benzamidine

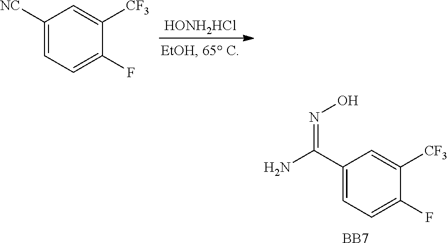

To a solution of the 4-fluoro-3-trifluoromethyl-benzonitrile (20.0 g, 105.8 mmol) in ethanol (400 mL) a solution of the NH₂OH.HCl (8.0 g, 116.4 mmol) which was adjusted to pH=7.0 by addition of NaOH in H₂O (8 mL) was added. The mixture was stirred vigorously and heated to about 65° C. for about 16 h. The analysis by TLC indicated the completion of the reaction; it was concentrated to remove the solvent. The residue was redissolved in DCM (200 mL) and washed with water, brine and dried over Na₂SO₄, concentrated in vacuum to afford 4-Fluoro-N-hydroxy-3-trifluoromethyl-benzamidine (BB7) as pale-yellow solid. ¹H NMR (400 MHz, DMSO) δ ppm 9.83 (1H, s), 8.00-7.98 (2H, m), 7.50 (1H, t) and 6.00 (2H, s).

### Preparation of BB10: 5-[3-(4-Fluoro-3-trifluoromethyl-phenyl)-[1,2,4]oxadiazol-5-yl]-2-(4-fluoro-piperidin-1-yl)-benzonitrile

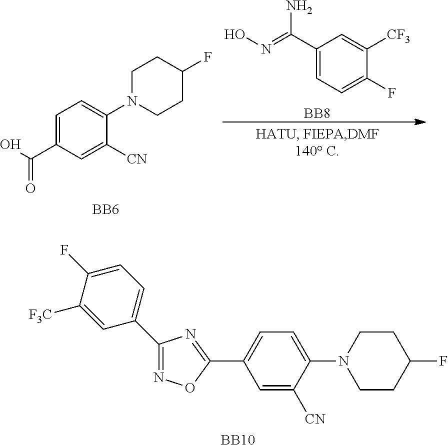

To a solution of compound BB6 (20 g, 80.6 mmol) in DMF (385 mL), the HATU (36.5 g, 96.7 mmol), 4-fluoro-N-hydroxy-3-trifluoromethyl-benzamidine (21.5 g, 96.7 mmol) and DIPA (29.3 g, 227 mmol) was added. The analysis by HPLC indicated there was no change starting material. The solution was cooled to about 25° C. and quenched by addition of H₂O/DCM (200 mL/500 mL). The organic layer was separated, washed with saturated NaHCO₃; brine (200 mL×2) dried over NaSO₄ and concentrated. The crude product was isolated by the pre-HPLC to afford the product as white solid (BB10) 1H NMR (400 MHz, DMSO) δ ppm 8.42-8.38 (2H, m), 8.32-8.30 (1H, m), 8.25-8.22 (1H, m), 7.75 (1H, t), 7.35 (1H, d), 5.01-4.87 (1H, m), 3.59-3.36 (4H, m), 2.12-2.02 (2H, m) and 1.92-1.74 (2H, m).

### Preparation #53 and 54

### Preparation of 5-(4-fluorophenyl)-3-(4-(4-fluoropiperidin-1-yl)-3-(trifluoromethyl)phenyl)-1,2,4-oxadiazole and 3-(4-(4,4-difluoropiperidin-1-yl)-3-(trifluoromethyl)phenyl)-5-(4-fluorophenyl)-1,2,4-oxadiazole

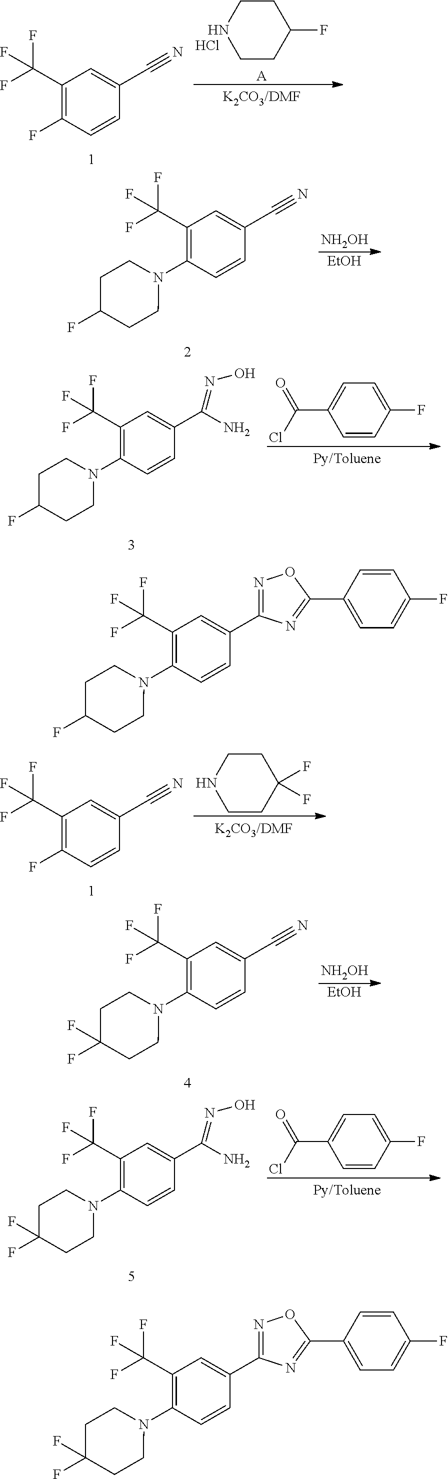

### Preparation of 4-(4-fluoropiperidin-1-yl)-3-(trifluoromethyl)benzonitrile (compound 2)

To a stirred solution of compound 1 (5.0 g, 0.026 mmol) and 4-fluoropiperidine.HCl (4.05 g, 0.029 mmol) in DMSO (50 mL), K₂CO₃ (9.12 g, 0.066 mmol) was added at RT. The reaction mixture was heated at about 100° C. for about 12 h. Upon complete consumption of starting material, the reaction mixture was cooled to RT and poured into crushed ice. The aqueous layer was extracted with hexane (2×50 mL), washed with brine (50 mL), dried over sodium sulfate and concentrated under reduced pressure to yield a crude compound which was purified by column chromatography using 2% EtOAc/Hexane to afford compound 2 as colorless liquid (4.75 g, 67%). ¹H NMR (200 MHz, CDCl₃): δ 1.94-2.01 (m, 2H), 2.04-2.12 (m, 2H), 2.92-3.03 (m, 2H), 3.13-3.25 (m, 2H), 4.70-5.10 (m, 1H), 7.32 (d, J=8.4 Hz, 1H), 7.75 (dd, J=8.4, 2.2 Hz, 1H), 7.89 (d, J=2.2 Hz, 1H);

MS (ESI): 273 (M⁺+H).

### Preparation of 4-(4-fluoropiperidin-1-yl)-N′-hydroxy-3-(trifluoromethyl)benzimidamide (compound 3)

A stirred solution of compound 2 (4.75 g, 0.017 mmol) in EtOH (50 mL), hydroxylamine (50% in water, 1.26 mL, 0.019 mmol) was added at RT. The reaction mixture was heated at about 65° C. for about 4 h. Upon complete consumption of starting material, the volatiles were removed under reduced pressure to afford crude 4-(4-fluoropiperidin-1-yl)-N′-hydroxy-3-(trifluoromethyl)benzimidamide 3 (5.4 g) which was used for the next step without further purification.

### Preparation of 5-(4-fluorophenyl)-3-(4-(4-fluoropiperidin-1-yl)-3-(trifluoromethyl)phenyl)-1,2,4-oxadiazole

A stirred solution of compound 3 (5.4 g, 0.017 mmol) in toluene (50 mL), 4-fluorobenzoyl chloride (2.33 mL, 0.019 mmol) and pyridine (1.56 mL, 0.019 mmol) were added at RT. The reaction mixture was heated at about 130° C. for about 10 h. Upon complete consumption of starting material the toluene was removed and the crude product was dissolved in EtOAc (100 mL). The organic layer was washed with water (50 mL), brine (50 mL), dried over sodium sulfate and concentrated under reduced pressure to get crude compound which was purified by column chromatography using 1% EtOAc/Hexane to afford 5-(4-fluorophenyl)-3-(4-(4-fluoropiperidin-1-yl)-3-(trifluoromethyl)phenyl)-1,2,4-oxadiazole as white solid (5.0 g, 69%).

¹H NMR (500 MHz, DMSO-d6): δ 1.83-1.90 (m, 2H), 1.95-2.04 (m, 2H), 2.49-2.94 (m, 2H), 3.07-3.11 (m, 2H), 4.80-4.92 (m, 1H), 7.49 (t, J=8.5 Hz, 2H), 7.68 (d, J=8.5 Hz, 1H), 8.24-8.29 (m, 4H).

¹³C NMR (125 MHz, DMSO-d6): δ 174.7, 166.9, 165.9, 163.9, 154.8, 131.8, 130.8, 125.8, 125.1, 124.8, 122.5, 121.8, 119.8, 116.8, 116.7, 88.2, 86.9, 49.4, 49.3, 31.5, 31.4.

### Preparation of 4-(4,4-difluoropiperidin-1-yl)-3-(trifluoromethyl)benzonitrile (compound 4)

A solution stirred solution of compound 1 (5.0 g, 0.026 mmol) and 4,4-difluoropiperidine.HCl (4.58 g, 0.029 mmol) in DMSO (50 mL), K₂CO₃ (9.12 g, 0.066 mmol) was added at RT. The reaction mixture was heated at about 100° C. for about 12 h. Upon complete consumption of starting material, the reaction mixture was cooled to RT and poured into crushed ice. The aqueous layer was extracted with EtOAc (2×100 mL), washed with water (60 mL), brine (60 mL), dried over sodium sulfate and concentrated under reduced pressure to get crude compound which was purified by column chromatography using 2% EtOAc/Hexane to get compound 4 as white solid (3.2 g, 42%). ¹H NMR (200 MHz, CDCl₃): δ 2.06-2.25 (m, 4H), 3.13 (t, J=5.4 Hz, 4H), 7.36 (d, J=8.4 Hz, 1H), 7.78 (dd, J=8.4, 2.2 Hz, 1H), 7.92 (d, J=2.2 Hz, 1H). MS (ESI): 290 (M⁺+H).

### Preparation of 4-(4,4-difluoropiperidin-1-yl)-N′-hydroxy-3-(trifluoromethyl)benzimidamide (compound 5)

A solution stirred solution of compound 4 (3.4 g, 0.011 mmol) in EtOH (65 mL), hydroxyl amine (50% in water, 0.85 mL, 0.025 mmol) was added at RT. The reaction mixture was heated at about 65° C. for about 3 h. Upon complete consumption of starting material, the volatiles were removed under reduced pressure to get crude compound 5 (3.78 g) which was used for the next step without further purification.

### Preparation of 3-(4-(4,4-difluoropiperidin-1-yl)-3-(trifluoromethyl)phenyl)-5-(4-fluorophenyl)-1,2,4-oxadiazole

A solution stirred solution of compound 5 (3.78 g, 0.011 mmol) in toluene (115 mL), 4-fluorobenzoyl chloride (1.54 mL, 0.013 mmol) and pyridine (1.03 mL, 0.013 mmol) were added at RT. The reaction mixture was heated at about 120° C. for about 10 h. Upon complete consumption of starting material, the toluene was removed and crude mass was dissolved in EtOAc (150 mL). The organic layer was washed with water (50 mL), brine (50 mL), dried over sodium sulfate and concentrated under reduced pressure to get crude compound which was purified by column chromatography using 1% EtOAc/Hexane to afford 3-(4-(4,4-difluoropiperidin-1-yl)-3-(trifluoromethyl)phenyl)-5-(4-fluorophenyl)-1,2,4-oxadiazole as white solid (2.5 g, 50%). ¹H NMR (500 MHz, CDCl₃): δ 2.13-2.20 (m, 4H), 3.11 (m, 4H), 7.23-7.44 (m, 2H), 7.48 (d, J=8.5 Hz, 1H), 8.22-8.26 (m, 2H), 8.30 (dd, J=8.5 Hz, 2.0 Hz, 1H), 8.45 (d, J=2.0 Hz, 1H) ¹³C NMR (125 MHz, CDCl₃): δ 175.1, 167.7, 166.6, 164.5, 154.2, 131.6, 130.7, 130.6, 127.4, 127.1, 127.0, 126.9, 126.8, 124.7, 124.2, 123.6, 123.4, 122.5, 121.5, 120.4, 119.6, 116.6, 116.4, 50.5, 50.48, 50.4, 34.7, 34.6, 34.4.

### Example #55

### Preparation of 1-(4-(3-(3-chloro-4-isopropoxyphenyl)-1,2,4-oxadiazol-5-yl)benzyl)-5-oxopyrrolidine-3-carboxylic acid

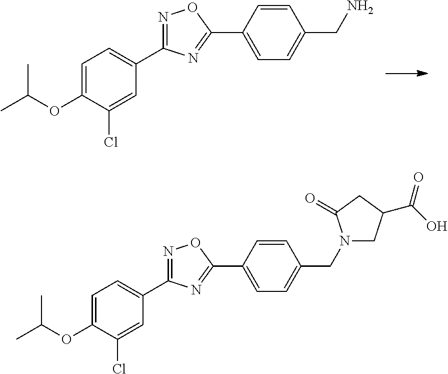

To a solution of (4-(3-(3-chloro-4-isopropoxyphenyl)-1,2,4-oxadiazol-5-yl)phenyl)methanamine (27 mg, 0.079 mmol) (Example #6) in MeOH (1 mL) was added dimethyl 2-methylenesuccinate (12.42 mg, 0.079 mmol). After about 4 h additional dimethyl 2-methylenesuccinate (124 mg, 0.79 mmol) was added and the reaction mixture was heated to about 50° C. After about 20 h the reaction mixture was cooled to RT and lithium hydroxide monohydrate (9.89 mg, 0.236 mmol) was added. After about 30 min a 1 N solution of HCl (10 mL) and DCM (10 mL) were added to the reaction mixture. The organic layer was removed, dried (Na₂SO₄) filtered and concentrated in vacuo. The oily residue was suspended in Et₂O (30 mL). After standing for about 30 min the resulting solid was collected by filtration and dried in vacuo to provide 1-(4-(3-(3-chloro-4-isopropoxyphenyl)-1,2,4-oxadiazol-5-yl)benzyl)-5-oxopyrrolidine-3-carboxylic acid (30 mg, 84%) as a colorless solid. LC/MS (Table 1, Method a) Rₜ=2.38 min, m/z 456 (M+H)⁺.

### Example #56

### Separation of (R)-3-(4-(3-(3-chloro-4-isopropoxyphenyl)-1,2,4-oxadiazol-5-yl)-1H-indol-1-yl)-2-methylpropanoic acid and (S)-3-(4-(3-(3-chloro-4-isopropoxyphenyl)-1,2,4-oxadiazol-5-yl)-1H-indol-1-yl)-2-methylpropanoic acid

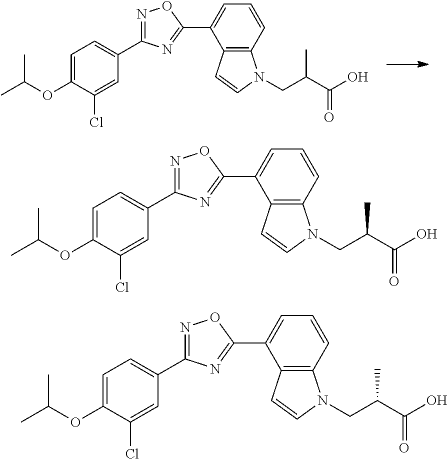

The enantiomers of 3-(4-(3-(3-chloro-4-isopropoxyphenyl)-1,2,4-oxadiazol-5-yl)-1H-indol-1-yl)-2-methylpropanoic acid (example #C. 3) were separated by chiral chromatography using a Daicel AD-H (20×250 mm, 5 μm particle) column at a flow rate of 25 mL/min, gradient elution with 2-16% ethanol in heptane with 0.2% diethylamine modifier over 26 min. Elution of compounds was monitored by UV detection at 254 nm with a column temperature of about 25° C. Injection samples were prepared by dissolving the racemic acid in 80% heptane: 20% ethanol: 0.2% diethylamine. Concentration of the fractions containing the desired products provided ((R)-3-(4-(3-(3-chloro-4-isopropoxyphenyl)-1,2,4-oxadiazol-5-yl)-1H-indol-1-yl)-2-methylpropanoic acid (stereochemistry was arbitrarily assigned) LC/MS (Table 1, Method a) Rₜ=3.05 min, m/z 440 (M+H)⁺, or =neg. Followed by ((S)-3-(4-(3-(3-chloro-4-isopropoxyphenyl)-1,2,4-oxadiazol-5-yl)-1H-indol-1-yl)-2-methylpropanoic acid (stereochemistry was arbitrarily assigned) LC/MS (Table 1, Method a) Rₜ=3.05 min, m/z 440 (M+H)⁺

### Preparation #57

### Preparation of (1R,2S)-methyl 2-(4-(3-(3-chloro-4-isopropoxyphenyl)-1,2,4-oxadiazol-5-yl)phenylamino)cyclopentanecarboxylate compound with (1S,2R)-methyl 2-(4-(3-(3-chloro-4-isopropoxyphenyl)-1,2,4-oxadiazol-5-yl)phenylamino)cyclopentanecarboxylate (1:1)

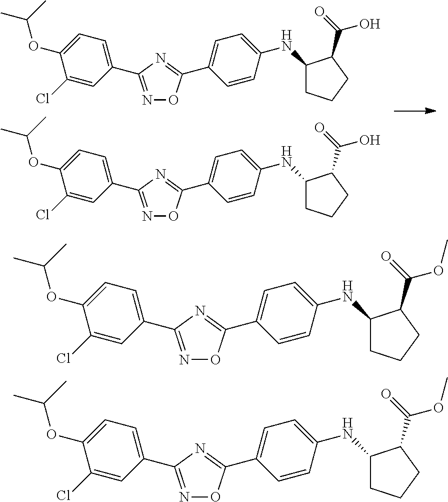

A 20 mL reaction vial was charged with (1R,2S)-2-(4-(3-(3-chloro-4-isopropoxyphenyl)-1,2,4-oxadiazol-5-yl)phenylamino)cyclopentanecarboxylic acid compound with (1S,2R)-2-(4-(3-(3-chloro-4-isopropoxyphenyl)-1,2,4-oxadiazol-5-yl)phenylamino) cyclopentanecarboxylic acid (1:1) (0.265 g, 0.300 mmol) in methanol (1.934 mL) to give a cloudy suspension. To the reaction mixture was added thionyl chloride (0.044 mL, 0.600 mmol) dropwise. The resulting solution was stirred at about 60° C. for about 15 hr. The mixture was cooled to about ambient temperature. The resulting white precipitate was filtered, rinsed with water (5 mL) and oven dried to provide (1R,2S)-methyl 2-(4-(3-(3-chloro-4-isopropoxyphenyl)-1,2,4-oxadiazol-5-yl)phenylamino)cyclopentanecarboxylate compound with (1S,2R)-methyl 2-(4-(3-(3-chloro-4-isopropoxyphenyl)-1,2,4-oxadiazol-5-yl)phenylamino)cyclopentanecarboxylate (1:1) (0.165 g, 0.181 mmol, 60.3% yield) as white solid. LC/MS (Table 1, Method a) Rₜ=3.11 min, m/z 456 (M+H)⁺.

### Example #58

### Preparation of (1R,2R)-2-(4-(3-(3-chloro-4-isopropoxyphenyl)-1,2,4-oxadiazol-5-yl)phenylamino)cyclopentanecarboxylic acid compound with (1S,2S)-2-(4-(3-(3-chloro-4-isopropoxyphenyl)-1,2,4-oxadiazol-5-yl)phenylamino)cyclopentanecarboxylic acid (1:1)

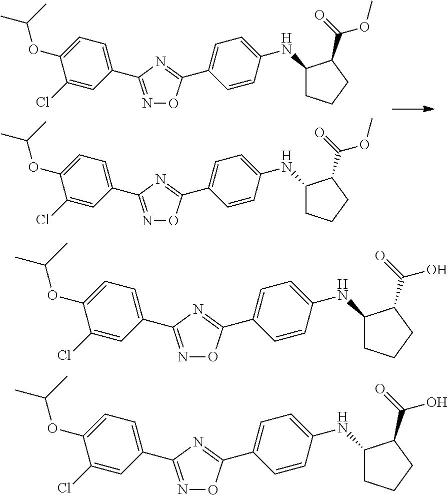

A nitrogen-flushed 25 mL flask was charged sodium (0.398 mg, 0.017 mmol). The flask was cooled to about 0° C. and methanol (5 mL) was added. The mixture was stirred until all metal dissolved. A solution of (1R,2S)-methyl 2-(4-(3-(3-chloro-4-isopropoxyphenyl)-1,2,4-oxadiazol-5-yl)phenylamino)cyclopentanecarboxylate compound with (1S,2R)-methyl 2-(4-(3-(3-chloro-4-isopropoxyphenyl)-1,2,4-oxadiazol-5-yl)phenylamino)cyclopentanecarboxylate (1:1) (0.158 g, 0.173 mmol) in methanol (10 mL) and DCM (3 mL) was added to the reaction mixture. After stirring at RT for about 48 h water (2 mL) was added to the reaction mixture. The mixture was concentrated in vacuo and the residue was partitioned between EtOAc (10 mL) and water (10 mL). The organic phase was dried (Na₂SO₄) filtered and concentrated. The crude material was purified by RP-HPLC on a Hyperprep HS C18 column using 0-95% ACN/Water (NH₄OAc buffer) over 30 min at 21 mL/min. Fractions were concentrated in vacuo to provide (1R,2R)-2-(4-(3-(3-chloro-4-isopropoxyphenyl)-1,2,4-oxadiazol-5-yl)phenylamino)cyclopentanecarboxylic acid compound with (1S,2S)-2-(4-(3-(3-chloro-4-isopropoxyphenyl)-1,2,4-oxadiazol-5-yl)phenylamino)cyclopentanecarboxylic acid (1:1) (0.0336 g, 0.038 mmol, 21.94% yield) as white solid. LC/MS (Table 1, Method a) Rₜ=2.76 min, m/z 442 (M+H)⁺.

### Example #59

### Preparation of 3-(3-chloro-4-isopropoxyphenyl)-5-(1-methyl-1,2,3,4-tetrahydroquinolin-6-yl)-1,2,4-oxadiazole

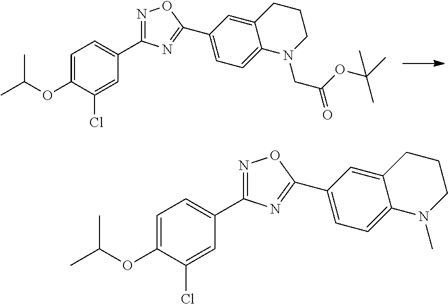

To a solution of tert-butyl 2-(6-(3-(3-chloro-4-isopropoxyphenyl)-1,2,4-oxadiazol-5-yl)-3,4-dihydroquinolin-1(21-1)-yl)acetate (0.117 g, 0.242 mmol) in DCM (2.015 mL) was added TFA (0.403 mL) dropwise. After about 24 h the reaction mixture was concentrated in vacuo and the crude material was purified by chromatography on silica gel (12 g) eluting with EtOAc/Heptane (0-15%). Fractions containing product were combined and concentrated to yield 3-(3-chloro-4-isopropoxyphenyl)-5-(1-methyl-1,2,3,4-tetrahydroquinolin-6-yl)-1,2,4-oxadiazole (0.0405 g, 0.106 mmol, 43.6% yield) as light yellow powder.

¹H NMR (400 MHz, DMSO) d 8.00 (s, 1H), 7.94 (d, J=8.7, 1H), 7.77 (d, J=8.7, 1H), 7.66 (s, 1H), 7.34 (d, J=8.7, 1H), 6.69 (d, J=8.8, 1H), 4.84-4.75 (m, 1H), 3.40-3.32 (m, 2H), 2.96 (s, 3H), 2.77 (t, J=6.1, 2H), 1.89 (s, 2H), 1.33 (d, J=6.0, 6H).

LC/MS (Table 1, Method a) Rₜ=3.26 min, m/z 384 (M+H)⁺.

### Preparation #58

### Preparation of (R)-5-(1H-indol-4-yl)-3-(4-(tetrahydrofuran-3-yloxy)-3-(trifluoromethyl)phenyl)-1,2,4-oxadiazole

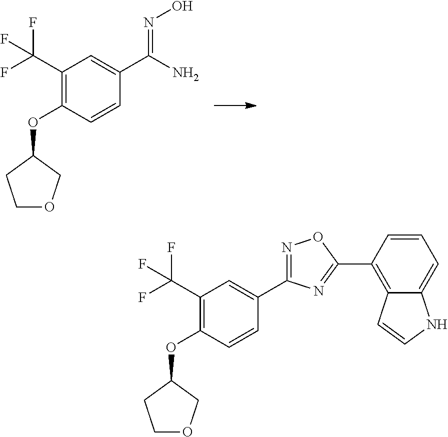

A mixture of 1H-indole-4-carboxylic acid (0.611 g, 3.79 mmol), EDCI hydrochloride (0.727 g, 3.79 mmol) and HOBT hydrate (0.580 g, 3.79 mmol) in anhydrous DMF (9.66 ml) was stirred at RT for about 1 hour under an atmosphere of nitrogen. A solution of (R)—N′-hydroxy-4-(tetrahydrofuran-3-yloxy)-3-(trifluoromethyl)benzimidamide (1.00 g, 3.45 mmol) in anhydrous DMF (1.823 ml) was added and the reaction mixture was stirred at approximately 140° C. for about 2 hr. The reaction was cooled to RT and poured into water (200 mL). The crude product was partitioned between EA and the aqueous phase. The combined organic extracts were washed with 20% brine solution and 1N HCl (2×50 mL), 1N NaOH (3×50 mL) and water (3×50 mL), then dried over MgSO₄, filtered and solvent removed to yield a pale brown solid 1.23 g of (R)-5-(1H-indol-4-yl)-3-(4-(tetrahydrofuran-3-yloxy)-3-(trifluoromethyl)phenyl)-1,2,4-oxadiazole LC/MS (Table 1, Method c) Rₜ=2.36 min, m/z 416.1 (M+H)⁺; ¹H NMR (400 MHz, DMSO-d₆) δ 11.7 (d, 1H), 8.39 (dd, 1H), 8.31 (d, 1H), 8.15 (s, 1H), 7.99 (dd, 1H), 7.79 (td, 1H), 7.69 (m, 1H), 7.53 (d, 1H), 7.35 (m, 1H), 7.18 (ddd, 1H), 5.36 (m, 1H), 3.82 (m, 4H), 2.31 (dd, 2H), 1.99 (s, 2H).

### Preparation #59

### Preparation of (R)-tert-butyl 3-(4-(3-(4-(tetrahydrofuran-3-yloxy)-3-(trifluoromethyl)phenyl)-1,2,4-oxadiazol-5-yl)-1H-indol-1-yl)propanoate

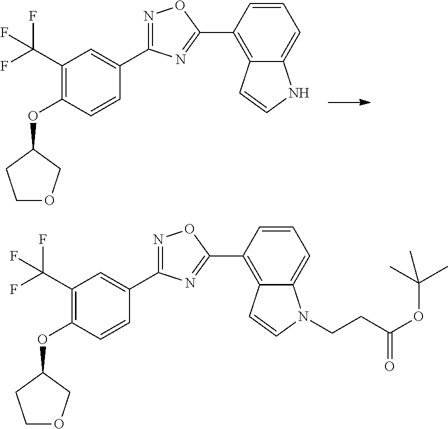

To a stirred solution of (R)-5-(1H-indol-4-yl)-3-(4-(tetrahydrofuran-3-yloxy)-3-(trifluoromethyl)phenyl)-1,2,4-oxadiazole (0.4 g, 0.963 mmol) in ACN (5.0 mL) (heated to about 60° C. till complete dissolution), tert-butyl acrylate (0.212 mL, 1.445 mmol) was added dropwise, followed by DBU (0.072 mL, 0.482 mmol). The solution was stirred at about 50° C. for about 18 h. The solvent was removed and the yellow solid was dissolved in EtOAc (150 mL), washed with saturated brine (3×100 mL), dried over MgSO₄, filtered and solvent removed to yield a yellow gum/solid (0.59 g). The gum/solid was triturated with EtOAc (5 mL), cooled to about 0-5° C. and the resultant pale yellow solid was collected, washed with ice-cold EtOAc (2×1 mL) and cold 30-60° C. petroleum ether (2×1 mL) and dried to afford (R)-tert-butyl 3-(4-(3-(4-(tetrahydrofuran-3-yloxy)-3-(trifluoromethyl)phenyl)-1,2,4-oxadiazol-5-yl)-1H-indol-1-yl)propanoate. (0.27 g, 52% yield). LC/MS (Table 1, Method c) Rₜ=2.76 min, m/z 544.22 (M+H)⁺; ¹H NMR (400 MHz, DMSO-d₆) δ 8.39 (dd, 1H), 8.3 (d, 1H), 8.01 (dd, 1H), 7.94 (d, 1H), 7.7 (d, 1H), 7.53 (d, 1H), 7.4 (m, 1H), 7.12 (dd, 1H), 5.36 (d, 1H), 4.52 (t, 2H), 3.99 (dd, 1H), 3.84 (ddd, 3H), 2.81 (t, 2H), 2.31 (dd, 1H), 2.07 (s, 1H), 1.31 (s, 9H).

### Example #60

### Preparation of (R)-3-(4-(3-(4-(tetrahydrofuran-3-yloxy)-3-(trifluoromethyl)phenyl)-1,2,4-oxadiazol-5-yl)-1H-indol-1-yl)propanoic acid

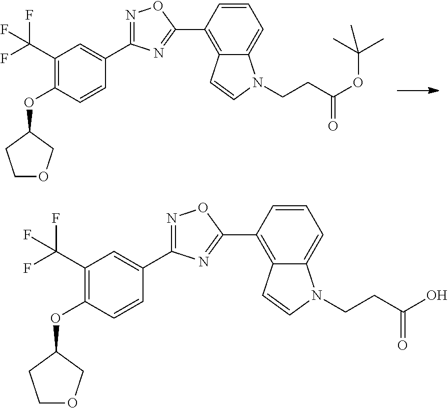

A stirred solution of (R)-tert-butyl 3-(4-(3-(4-(tetrahydrofuran-3-yloxy)-3-(trifluoromethyl)phenyl)-1,2,4-oxadiazol-5-yl)-1H-indol-1-yl)propanoate (0.25 g, 0.460 mmol) in DCM (23 mL) was treated with trifluoroacetic acid (0.709 mL, 9.20 mmol) at ambient temperature. The reaction mixture was stirred for about 20 h. The solvent was removed and the residue was diluted with water (50 mL). The product was partitioned between EtOAc (4×2 mL) and the acid aqueous phase. The extracts were combined and washed with saturated brine (4×30 mL) and dried over MgSO₄, filtered and the solvent removed in vacuo to yield a pale yellow solid (0.24 g). The solid was recrystallized from can (8 mL), cooled to ambient temperature before the solid was collected, washed with ACN (2×2 mL), 30-60° C. petroleum ether (2×2 mL) and dried to give a pale yellow powdery solid of (R)-3-(4-(3-(4-(tetrahydrofuran-3-yloxy)-3-(trifluoromethyl)phenyl)-1,2,4-oxadiazol-5-yl)-1H-indol-1-yl)propanoic acid. (0.155 g, 69%) LC/MS (Table 1, Method c) Rₜ=2.12 min, m/z 486.11 (M−H)⁻. ¹H NMR (400 MHz, DMSO-d₆) δ 12.48 (m, 1), 8.38 (dd, 1H), 8.3 (d, 1H), 8.01 (dd, 1H), 7.95 (d, 1H), 7.71 (d, 1H), 7.53 (d, 1H), 7.4 (m, 1H), 7.16 (dd, 1H), 5.36 (dt, 1H), 4.53 (t, 2H), 3.99 (dd, 1H), 3.84 (ddd, 3H), 2.81 (t, 2H), 2.31 (m, 1H), 2.05 (m, 1H).

### Examples #61, 62, 63 and 64

### Preparation of (1R,3S)-3-[4-(5-biphenyl-2-yl-[1,2,4]oxadiazol-3-yl)-2-methyl-phenylamino]-cyclopentanecarboxylic acid, (1R,3S)-3-[4-(5-biphenyl-3-yl-[1,2,4]oxadiazol-3-yl)-2-methyl-phenylamino]-cyclopentanecarboxylic acid, (1R,3S)-3-[4-(5-biphenyl-4-yl-[1,2,4]oxadiazol-3-yl)-2-methyl-phenylamino]-cyclopentanecarboxylic acid and (1R,3S)-3-{4-[5-(4-cyclohexyl-phenyl)-[1,2,4]oxadiazol-3-yl]-2-methyl-phenylamino}-cyclopentanecarboxylic acid

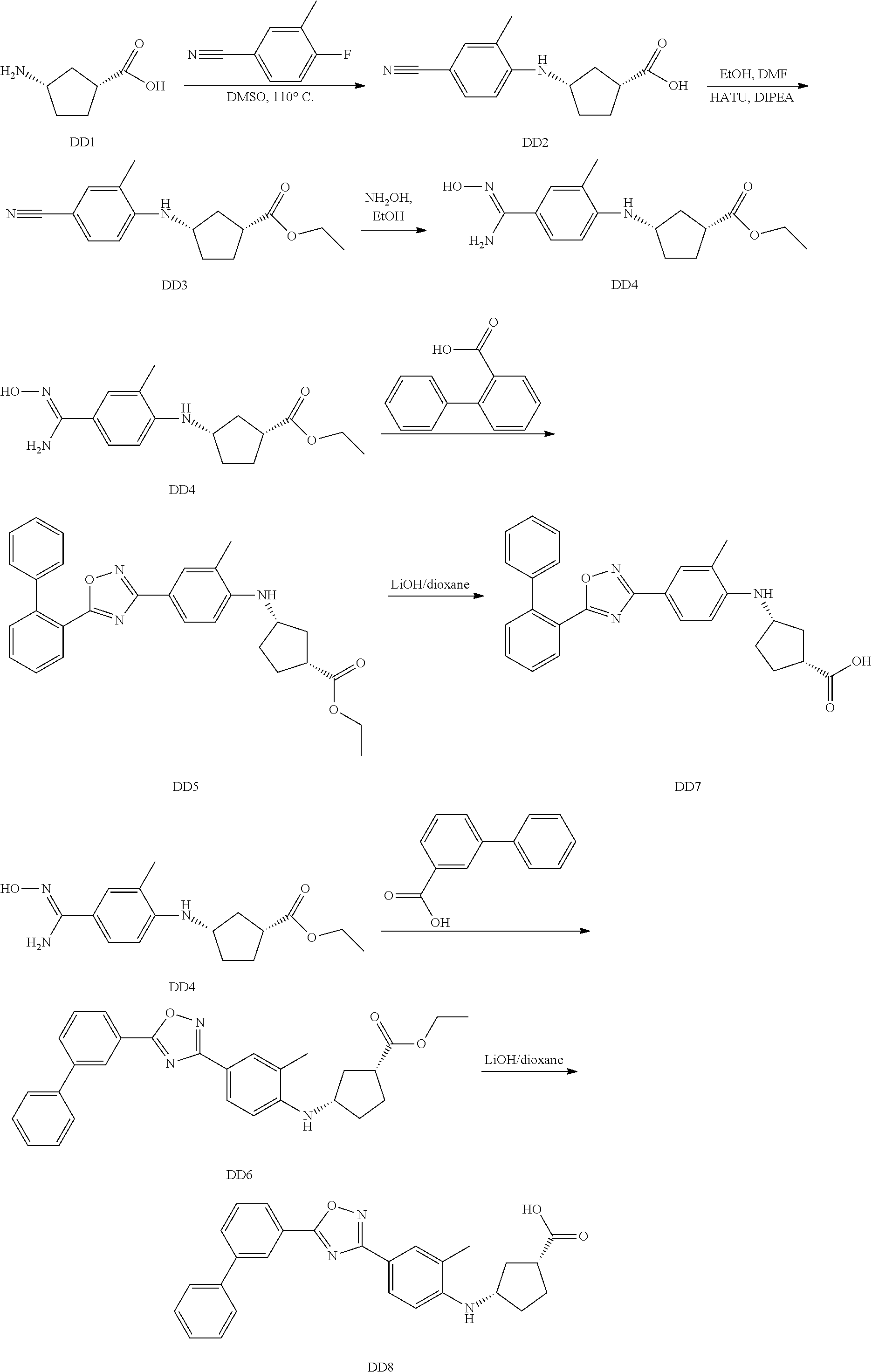

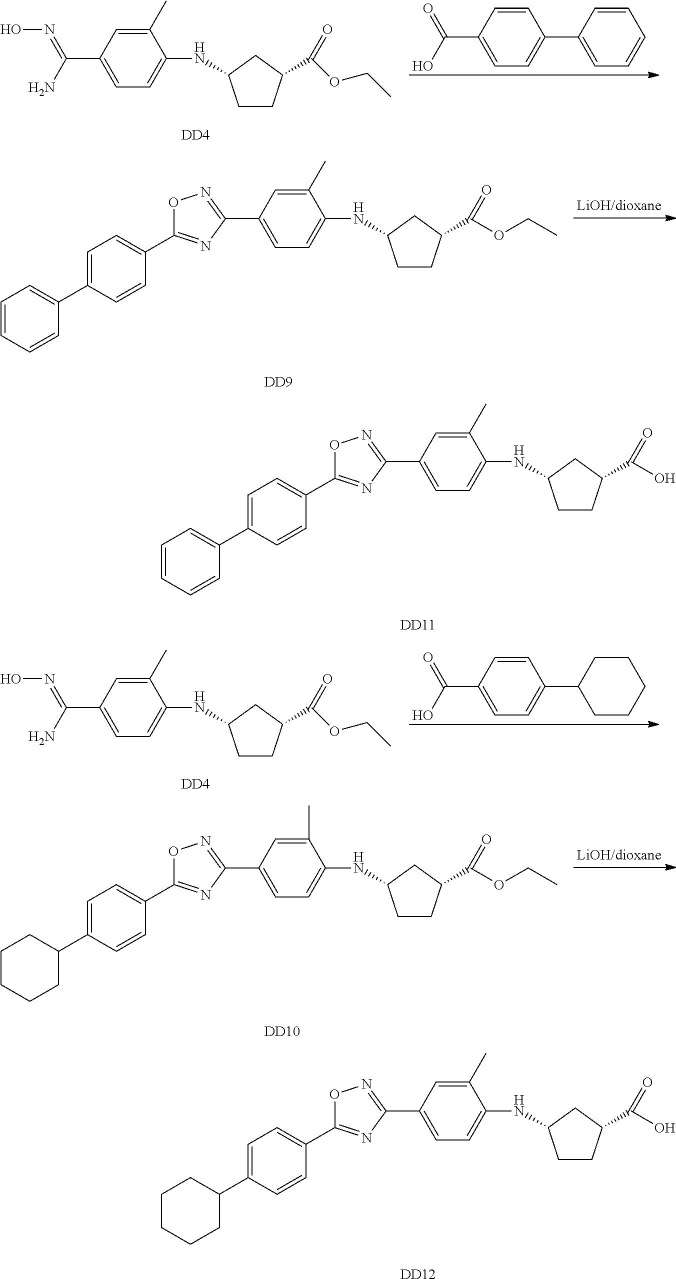

### Synthesis of DD2: (1R,3S)-3-(4-Cyano-2-methyl-phenylamino)-cyclopentanecarboxylic acid

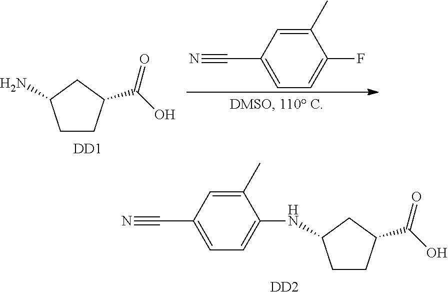

A solution of DD1 (1R,3S)-3-Amino-cyclopentanecarboxylic acid (5.89 g, 45.6 mmol) and 4-fluoro-3-methylbenzonitrile (5.60 g, 41.4 mmol) in DMSO (140 mL) and water (11.67 mL) was treated with potassium carbonate (12.60 g, 91 mmol) and the mixture was heated to about 110° C. under nitrogen overnight. The solution was cooled to room temperature and diluted with water, washed with ether then acidified to pH=2 with concentrated HCl. The product was extracted with ethyl acetate, the combined ethyl acetate layers were washed with saturated NaCl solution, dried over sodium sulfate, filtered and concentrated to about 100 mL. Hexane (100 mL) was added and the solution concentrated until product comes out of solution. Filtered off solids and concentrated the filtrate to a gum which was dissolved in methanol 40 mL, and water and concentrated until crystals formed. A second crop of solids was filtered off combined with the first crop then dried in a vacuum overnight to give 5.78 g of (1R,3S)-3-(4-Cyano-2-methyl-phenylamino)-cyclopentanecarboxylic acid (DD2, 57% yield) LC/MS (ESI): m/z 245.2 (M+H), Rₜ: 0.78 min.

### Synthesis of DD3: (1R,3S)-3-(4-Cyano-2-methyl-phenylamino)-cyclopentanecarboxylic acid ethyl ester

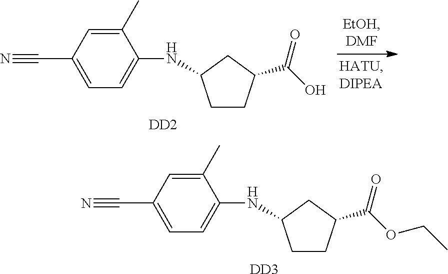

In a 500 mL round bottom flask, (1R,3S)-3-(4-Cyano-2-methyl-phenylamino)-cyclopentanecarboxylic acid (DD2, 5.78 g, 23.7 mmol) in DMF (200 mL) was added to give orange solution. Molecule sieve (4A, 8-12 mesh, 4.4 g) was added. HATU (10.8 g, 28.4 mmol) was added in one portion, ethanol (2.08 mL, 35.6 mmol) was added. The solution was stirred at RT for about 30 min, then the mixture was cooled to about 0° C. in an ice bath. N,N-ciisopropylethylamine (8.27 mL, 47.4 mmol) was added dropwise over about 10 min. The ice-bath was removed, and the solution was stirred at RT overnight. The reaction mixture was filtered, washed with DMF, the combined filtrate was concentrated, the residue was partitioned between EtOAc and sat ammonium chloride, the organic layer was washed with saturated ammonium chloride, water, saturated sodium bicarbonate, and brine, dried over sodium sulfate, filtered and concentrated afforded 8.1 g of brown oil, which was purified by gel chromatography (0-30% EtOAc/petroleum ether) to afford (1R,3S)-3-(4-Cyano-2-methyl-phenylamino)-cyclopentanecarboxylic acid ethyl ester (DD3, 5.48 g, 20.1 mmol, 85% yield) as orange oil. LC/MS (ESI): m/z 273.3 (M+H), Rₜ: 0.928 min.

### Synthesis of DD4: (1R,3S)-3-[4-(N-Hydroxycarbamimidoyl)-2-methyl-phenylamino]-cyclopentanecarboxylic acid ethyl ester

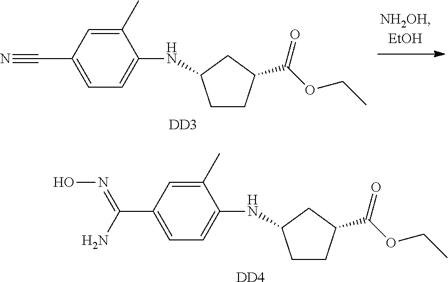

In a 250 mL round bottom flask, (1R,3S)-3-(4-Cyano-2-methyl-phenylamino)-cyclopentanecarboxylic acid ethyl ester (DD3, 5.48 g, 20.1 mmol) and hydroxylamine (4.92 mL, 80.4 mmol) in EtOH (86 mL) were added to give pale yellow solution. The solution was heated at about 50° C. overnight. The reaction mixture was near colorless solution, which was concentrated, the residue was loaded into silica gel cartridge with the aid of EtOAc, purified by HPLC reverse phase (0% over 3 min, 0-95% MeOH/H₂O over 30 min; 220 g silica gel column) to afford (1R,3S)-3-[4-(N-Hydroxycarbamimidoyl)-2-methyl-phenylamino]-cyclopentanecarboxylic acid ethyl ester (DD4, 4 g, 13.1 mmol, 65% yield) as off-white slight waxy solid. LC/MS (ESI): m/z 306 (M+H), Rₜ:1.80 min.

### Synthesis of DD5: (1R,3S)-3-[4-(5-Biphenyl-2-yl-[1,2,4]oxadiazol-3-yl)-2-methyl-phenylamino]-cyclopentanecarboxylic acid ethyl ester

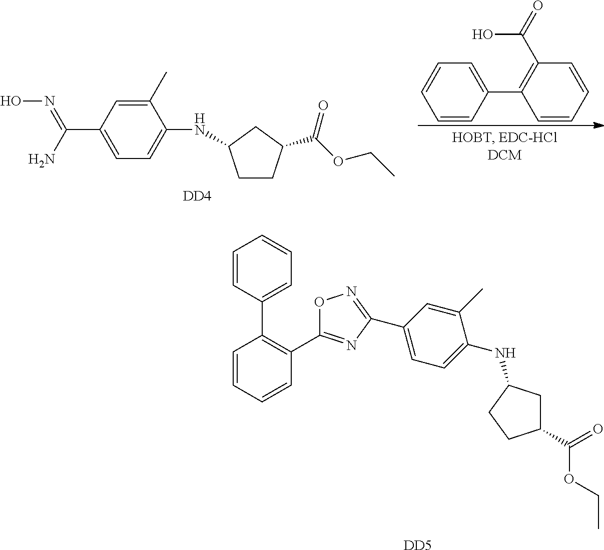

In a 250 mL round bottom flask, 2-biphenylcarboxylic acid (340 mg, 1.72 mmol) and HOBT (300 mg, 1.97 mmol) in DCM (5 mL) were added to give white suspension. EDC-HCl (377 mg, 1.97 mmol) was added in one portion, the reaction mixture became mostly clear, then it rapidly became a cloudy suspension. After about 1 h, the reaction mixture was a cloudy pale yellow suspension. (1R,3S)-3-[4-(N-Hydroxycarbamimidoyl)-2-methyl-phenylamino]-cyclopentanecarboxylic acid ethyl ester (DD4, 500 mg, 1.64 mmol) was added with the aid of 5 mL DCM, and the reaction mixture became deep blue solution immediately. It was stirred at RT for about 150 min. The reaction mixture was diluted with DCM, washed with brine, dried over sodium sulfate overnight, filtered and concentrated to afford a waxy yellow solid, which was dissolved in 1,4-dioxane (10 mL) to form a deep red solution, Molecular sieves (4 Å, 8-12 mesh, beads, 300 mg) were added. The solution was heated at about 110° C. for about 2 h. The solution was cooled down, the reaction mixture was a black solution, it was concentrated, the residue was purified via silica gel chromatography (6/1 petroleum ether to EtOAc) to afford 110 mg (14.4% yield) of (1R,3S)-3-[4-(5-Biphenyl-2-yl-[1,2,4]oxadiazol-3-yl)-2-methyl-phenylamino]-cyclopentanecarboxylic acid ethyl ester DD5. LC/MS (ESI): m/z 468.2 (M+H), Rₜ: 2.49 min.

### Synthesis of DD6: (1R,3S)-3-[4-(5-Biphenyl-3-yl-[1,2,4]oxadiazol-3-yl)-2-methyl-phenylamino]-cyclopentanecarboxylic acid ethyl ester

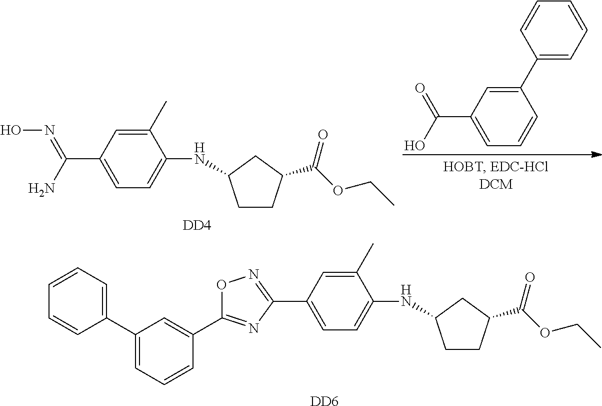

In a 250 mL round bottom flask, 3-biphenylcarboxylic acid (340 mg, 1.72 mmol) and HOBT (300 mg, 1.97 mmol) in DCM (5 mL) were added to give a white suspension. EDC-HCl (377 mg, 1.97 mmol) was added in one portion, the reaction mixture became mostly clear, then it rapidly became a cloudy suspension. After about 1 h, the reaction mixture was a cloudy pale yellow suspension. (1R,3S)-3-[4-(N-Hydroxycarbamimidoyl)-2-methyl-phenylamino]-cyclopentanecarboxylic acid ethyl ester (DD10, 500 mg, 1.64 mmol) was added with the aid of 5 mL DCM. The reaction mixture became deep blue solution immediately, it was stirred at RT for about 150 min. The reaction mixture was diluted with DCM, washed with brine, dried over sodium sulfate overnight, filtered and concentrated to afford a waxy yellow solid, which was dissolved in 1,4-dioxane (10 mL) to form a deep red solution, MS (4 Å, 8-12 mesh, beads, 300 mg) was added. The solution was heated at about 110° C. for about 2 h. The solution was cooled down, the reaction mixture was a black solution, it was concentrated, the residue was purified via silica gel chromatography (6/1 petroleum ether to EA) to afford 280 mg (36.6% yield) of compound DD6: (1R,3S)-3-[4-(5-Biphenyl-3-yl-[1,2,4]oxadiazol-3-yl)-2-methyl-phenylamino]-cyclopentanecarboxylic acid ethyl ester

LC/MS (ESI): m/z 468.2 (M+H), Rₜ: 2.57 min.

### Synthesis of DD9: (1R,3S)-3-[4-(5-Biphenyl-4-yl-[1,2,4]oxadiazol-3-yl)-2-methyl-phenylamino]-cyclopentanecarboxylic acid ethyl ester

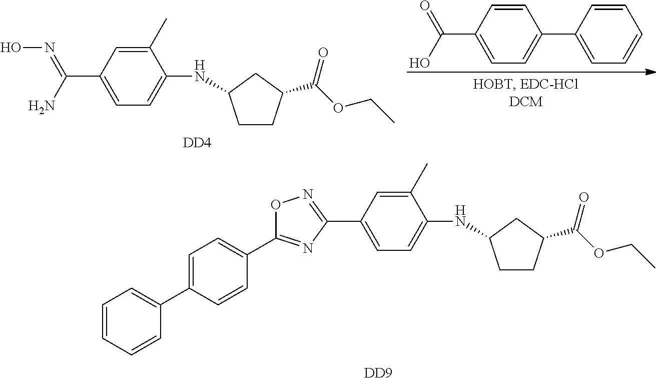

In a 250 mL round bottom flask, 4-biphenylcarboxylic acid (340 mg, 1.72 mmol) and HOBT (300 mg, 1.97 mmol) in DCM (5 mL) were added to give a white suspension. EDC-HCl (377 mg, 1.97 mmol) was added in one portion, the reaction mixture became mostly clear, then it rapidly became a cloudy suspension. After about 1 h, the reaction mixture was a cloudy pale yellow suspension. (1R,3S)-3-[4-(N-hydroxycarbamimidoyl)-2-methyl-phenylamino]-cyclopentanecarboxylic acid ethyl ester (DD4, 500 mg, 1.64 mmol) was added with the aid of 5 mL DCM. The reaction mixture became deep blue solution immediately, it was stirred at RT for about 150 min. The reaction mixture was diluted with DCM, washed with brine, dried over sodium sulfate overnight, filtered and concentrated to afford a waxy yellow solid, which was dissolved in 1,4-dioxane (10 mL) to form a deep red solution, MS (4 Å, 8-12 mesh, beads, 300 mg) was added. The solution was heated at about 110° C. for about 2 h. The solution was cooled down, the reaction mixture was black solution, it was concentrated, the residue was purified via silica gel chromatography (6/1 petroleum ether to EA) to afford 320 mg (41.8% yield) of (1R,3S)-3-[4-(5-Biphenyl-4-yl-[1,2,4]oxadiazol-3-yl)-2-methyl-phenylamino]-cyclopentanecarboxylic acid ethyl ester DD9 LC/MS (ESI): m/z 468.2 (M+H), Rₜ: 2.57 min.

### Synthesis of DD10: (1R,3S)-3-{4-[5-(4-Cyclohexyl-phenyl)-[1,2,4]oxadiazol-3-yl]-2-methyl-phenylamino}-cyclopentanecarboxylic acid ethyl ester

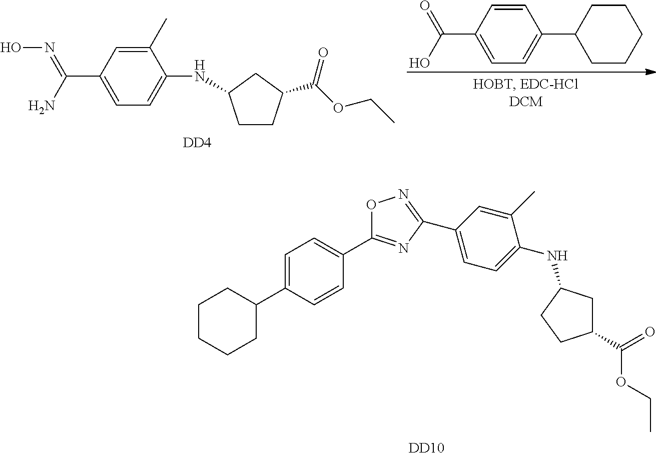

In a 250 mL round bottom flask, 4-cyclohexyl benzoic acid (340 mg, 1.72 mmol) and HOBt (300 mg, 1.97 mmol) in DCM (5 m″) were added to give white suspension. EDC-HCl (377 mg, 1.97 mmol) was added in one portion, the reaction mixture became mostly clear, then it rapidly became a cloudy suspension. After about 1 h, the reaction mixture was a cloudy pale yellow suspension. (1R,3S)-3-[4-(N-hydroxycarbamimidoyl)-2-methyl-phenylamino]-cyclopentanecarboxylic acid ethyl ester (DD4, 500 mg, 1.64 mmol) was added with the aid of 5 mL DCM. The reaction mixture became deep blue solution immediately, it was stirred at RT for about 150 min. The reaction mixture was diluted with DCM, washed with brine, dried over sodium sulfate overnight, filtered and concentrated to afforded a waxy yellow solid, which was dissolved in 1,4-dioxane (10 mL) to form a deep red solution, MS (4 Å, 8-12 mesh, beads, 300 mg) was added. The solution was heated at about 110° C. for about 2 h. The solution was cooled down, the reaction mixture was black solution, it was concentrated, the residue was purified via silica gel chromatography (6/1 petroleum ether to EA) to afford 91 mg (11.3% yield) of compound DD10: (1R,3S)-3-{4-[5-(4-Cyclohexyl-phenyl)-[1,2,4]oxadiazol-3-yl]-2-methyl-phenylamino}-cyclopentanecarboxylic acid ethyl ester

LC/MS (ESI): m/z 474.2 (M+H), Rₜ: 2.75 min.

### Example #61

### Preparation of DD7: (1R,3S)-3-[4-(5-biphenyl-2-yl-[1,2,4]oxadiazol-3-yl)-2-methyl-phenylamino]-cyclopentanecarboxylic acid

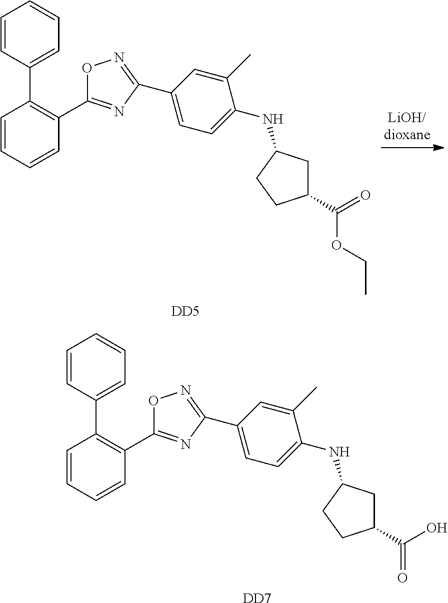

A solution of DDS: (1R,3S)-3-[4-(5-Biphenyl-2-yl-[1,2,4]oxadiazol-3-yl)-2-methyl-phenylamino]-cyclopentanecarboxylic acid ethyl ester (110 mg, 0.235 mmol) in 1,4-dioxane (10 mL) with LiOH (1M, 4 mL), stirred at RT for about 4 h, then the mixture was acidified by 1.0 M HCl, diluted with EtOAc, washed by brine to pH=about 6, dried over Na₂SO₄, and concentrated. HPLC reverse phase purification gave 48.5 mg (47% yield) of DD7 (1R,3S)-3-[4-(5-biphenyl-2-yl-[1,2,4]oxadiazol-3-yl)-2-methyl-phenylamino]-cyclopentanecarboxylic acid. ¹HNMR (500 MHz, CDCl₃, δ) 8.06 (d, 1H, J=7.6 Hz), 67.78 (d, 1H, J=8.4 Hz), 7.73 (s, 1H), 7.61 (t, 1H, J=7.0 Hz), 7.52-7.48 (m, 2H), 7.36-7.35 (m, 3H), 7.29-7.26 (m, 2H), 6.65 (d, 1H, J=8.4 Hz), 5.17 (br, 1H), 4.05-4.03 (m, 1H), 3.01-2.99 (m, 1H), 2.38-2.29 (m, 1H), 2.14 (s, 3H), 2.10-1.85 (m, 5H).

### Example #62

### Preparation of DD8: (1R,3S)-3-[4-(5-biphenyl-3-yl-[1,2,4]oxadiazol-3-yl)-2-methyl-phenylamino]-cyclopentanecarboxylic acid

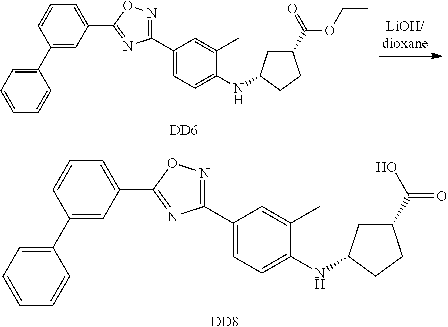

A solution of DD6: (1R,3S)-3-[4-(5-biphenyl-3-yl-[1,2,4]oxadiazol-3-yl)-2-methyl-phenylamino]-cyclopentanecarboxylic acid ethyl ester (280 mg, 0.599 mmol) in 1,4-dioxane (3 mL) with LiOH (1M, 2.4 mL), stirred at RT for about 4 h, then the mixture was acidified by 1.0 M HCl, diluted with EtOAc, washed by brine to pH about 6, dried over Na₂SO₄, and concentrated. HPLC reverse phase purification gave 144.9 mg (55.1% yield) of compound DD8: (1R,3S)-3-[4-(5-Biphenyl-3-yl-[1,2,4]oxadiazol-3-yl)-2-methyl-phenylamino]-cyclopentanecarboxylic acid. ¹HNMR (500 MHz, CDCl₃, δ) 8.43 (s, 1H), 8.18 (d, 1H, J=8.4 Hz), 7.95 (d, 1H, J=8.4 Hz), 7.88 (s, 1H), 7.81 (d, 1H, J=8.0 Hz), 7.68 (d, 2H, J=7.2 Hz), 7.61 (t, 1H, J=7.8 Hz), 7.50 (t, 2H, J=7.6 Hz), 7.41 (t, 1H, J=7.4 Hz), 6.70 (d, 1H, J=4.4 Hz), 3.07-3.00 (m, 1H), 2.42-2.33 (m, 1H), 2.19 (s, 3H), 2.14-1.88 (m, 5H).

### Example #63

### Preparation of DD11: (1R,3S)-3-[4-(5-biphenyl-4-yl-[1,2,4]oxadiazol-3-yl)-2-methyl-phenylamino]-cyclopentanecarboxylic acid

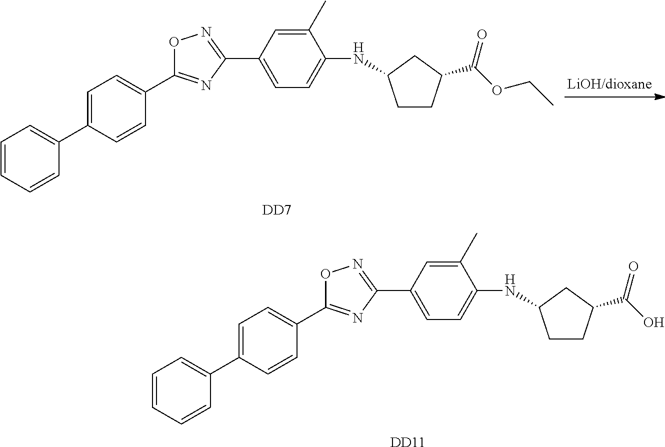

A solution of (1R,3S)-3-[4-(5-Biphenyl-4-yl-[1,2,4]oxadiazol-3-yl)-2-methyl-phenylamino]-cyclopentanecarboxylic acid ethyl ester DD9 (105 mg, 0.225 mmol) in 1,4-dioxane (3 mL) with LiOH (1M, 0.9 mL), stirred at RT for about 4 h, then the mixture was acidified by 1.0 M HCl, diluted with EA, washed by brine to about pH=6, dried over Na₂SO₄, concentrated. HPLC reverse phase purification gave 90.9 mg (92% yield) of compound DD11: (1R,3S)-3-[4-(5-Biphenyl-4-yl-[1,2,4]oxadiazol-3-yl)-2-methyl-phenylamino]-cyclopentanecarboxylic acid. ¹HNMR (500 MHz, CDCl₃, δ) 8.26 (d, 1H, J=8.4 Hz), 7.96 (d, 1H, J=8.4 Hz), 7.89 (s, 1H), 7.75 (d, 2H, J=8.4 Hz), 7.65 (d, 2H, J=7.2 Hz), 7.48 (t, 1H, J=7.4 Hz), 7.41 (t, 2H, J=7.4 Hz), 6.76 (d, 1H, J=8.8 Hz), 6.16 (br, 1H), 4.13-4.05 (m, 1H), 3.07-3.00 (m, 1H), 2.40-2.33 (m, 1H), 2.22 (s, 3H), 2.15-1.88 (m, 5H).

### Example #64

### Preparation of DD12: (1R,3S)-3-{4-[5-(4-Cyclohexyl-phenyl)-[1,2,4]oxadiazol-3-yl]-2-methyl-phenylamino}-cyclopentanecarboxylic acid

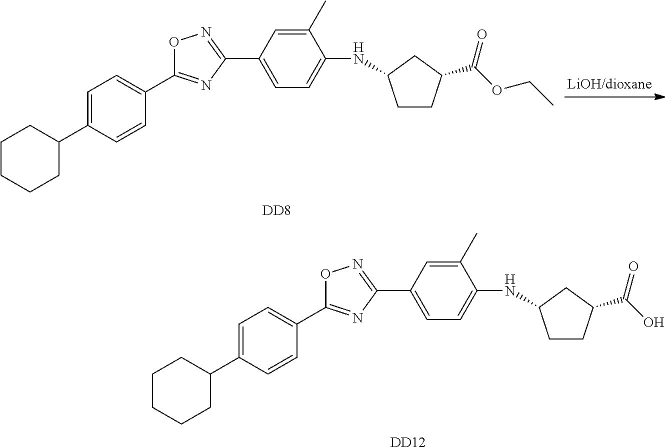

A solution of DD10: (1R,3S)-3-{4-[5-(4-cyclohexylcyclohexyl-phenyl)-[1,2,4]oxadiazol-3-yl]-2-methyl-phenylamino}-cyclopentanecarboxylic acid ethyl ester (129 mg, 0.272 mmol) in dioxane (10 mL) with LiOH (1M, 1.1 mL), was stirred at RT for about 4 h, then the mixture was acidified by 1.0 M HCl, diluted with EtOAc, washed by brine to about pH=6, dried over Na₂SO₄, and concentrated. HPLC reverse phase purification gave 97.1 mg (80% yield) of compound DD12: (1R,3S)-3-{4-[5-(4-Cyclohexyl-phenyl)-[1,2,4]oxadiazol-3-yl]-2-methyl-phenylamino}-cyclopentanecarboxylic acid. ¹HNMR (500 MHz, CDCl₃, δ) 8.10 (d, 1H, J=8.4 Hz), 7.93 (d, 1H, J=7.6 Hz), 7.89 (s, 1H), 7.36 (d, 2H, J=8.4 Hz), 6.82 (d, 1H, J=8.0 Hz), 6.62 (br, 1H), 4.13-4.04 (m, 1H), 3.10-2.97 (m, 1H), 3.61-2.56 (m, 1H), 2.38-2.31 (m, 1H), 2.24 (s, 3H), 2.11-1.75 (m, 10H), 1.50-1.25 (m, 1H).

### Example #65

### Preparation of (1R,3S)-3-((4-(3-(3-chloro-4-isopropoxyphenyl)-1,2,4-oxadiazol-5-yl)phenyl)(methyl)amino)cyclopentanecarboxylic acid

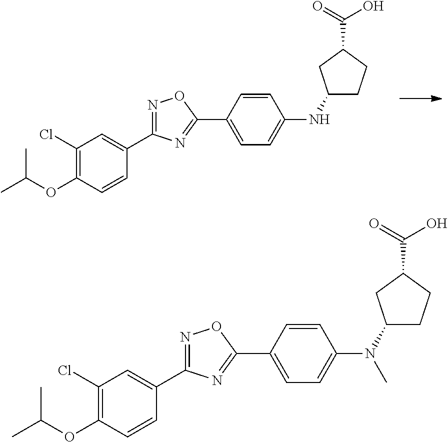

A 20 mL reaction vial equipped with septa cap outfitted with nitrogen inlet needle was charged with (1R,3S)-3-(4-(3-(3-chloro-4-isopropoxyphenyl)-1,2,4-oxadiazol-5-yl)phenylamino)cyclopentanecarboxylic acid (0.469 g, 1.061 mmol) and formaldehyde solution (0.398 mL, 5.31 mmol) in acetic acid (10.61 mL) to give a colorless suspension. Sodium triacetoxyborohydride (0.675 g, 3.18 mmol) was added in one portion. The resulting suspension was allowed to stir at RT for 1 day. The reaction mixture was concentrated. The sample was diluted with water and the resulting solid was collected by filtration and dried open to the air. The sample was further purified via automated silica gel chromatography (5% MeOH:CH₂Cl₂; EA-40 g column, 18×150 mm test tubes). The fractions containing product were concentrated to give (1R,3S)-3-((4-(3-(3-chloro-4-isopropoxyphenyl)-1,2,4-oxadiazol-5-yl)phenyl)(methyl)amino)cyclopentanecarboxylic acid (101 mg, 21%) as a white solid.

LC/MS (Table 1, Method g) Rₜ=3.08 min, m/z 456 (M+H)⁺; ¹H NMR (400 MHz, DMSO) δ 8.04-7.82 (m, 4H), 7.35 (d, J=8.9, 1H), 6.96 (d, J=9.2, 2H), 4.80 (dt, J=12.2, 6.0, 1H), 4.46 (dd, J=17.1, 7.4, 1H), 2.87 (s, 3H), 2.84-2.74 (m, 1H), 2.16-2.05 (m, 1H), 2.05-1.60 (m, 6H), 1.33 (d, J=6.0, 6H)

### Preparation #60a

### Preparation of Methyl 3-(5-(3-(3-chloro-4-isopropoxyphenyl)-1,2,4-oxadiazol-5-yl)-1H-indol-1-yl)cyclopentanecarboxylate

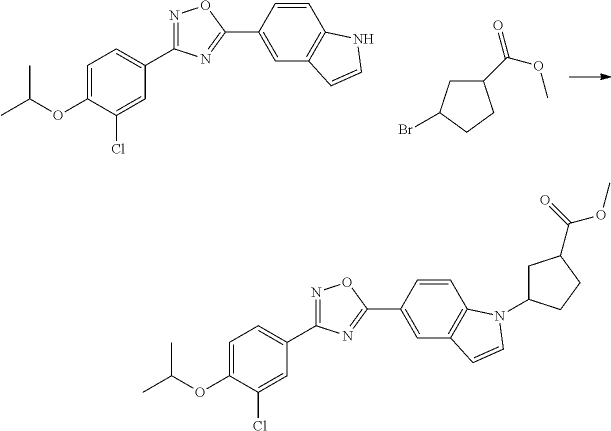

A 100 mL round bottom flask equipped with rubber septum and nitrogen inlet needle was charged with 3-(3-chloro-4-isopropoxyphenyl)-5-(1H-indol-5-yl)-1,2,4-oxadiazole (0.144 g, 0.407 mmol) in DMF (4.07 mL) to give a tan solution. The reaction mixture was cooled at about 0° C. for about 10 min. Sodium hydride dispersion in mineral oil (0.020 g, 0.488 mmol) was added in one portion. The resulting suspension was allowed to stir at about 0° C. for about 20 min. Methyl 3-bromocyclopentanecarboxylate (0.101 g, 0.488 mmol) was added in one portion. The resulting yellow solution was allowed to stir at RT for 1 day. Additional methyl 3-bromocyclopentanecarboxylate (0.169 g, 0.814 mmol) and sodium iodide (0.031 g, 0.204 mmol) were each added sequentially in one portion. The resulting solution was allowed to stir at RT for an additional 18 h. The reaction mixture was concentrated and the sample was transferred in solution onto a prep HPLC column and eluted with 60%-100% MeCN:Water (NH₄OAc buffer) over 30 min. The fractions containing product were combined and concentrated to afford methyl 3-(5-(3-(3-chloro-4-isopropoxyphenyl)-1,2,4-oxadiazol-5-yl)-1H-indol-1-yl)cyclopentanecarboxylate (11 mg, 6%). LC/MS (Table 1, Method g) Rₜ=3.55 min, m/z 480 (M+H)⁺.

### Example #67

### Preparation of 3-(5-(3-(3-chloro-4-isopropoxyphenyl)-1,2,4-oxadiazol-5-yl)-1H-indol-1-yl)cyclopentanecarboxylic acid

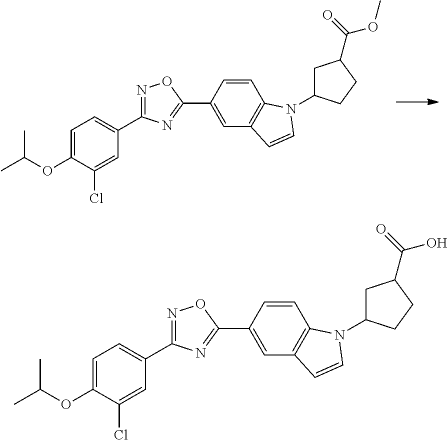

A 25 mL round bottom flask equipped with rubber septum and nitrogen inlet needle was charged with methyl 3-(5-(3-(3-chloro-4-isopropoxyphenyl)-1,2,4-oxadiazol-5-yl)-1H-indol-1-yl)cyclopentanecarboxylate (0.011 g, 0.023 mmol) in (1,4?)dioxane (2.292 mL) to give a colorless solution. Sodium hydroxide solution (0.229 mL, 0.229 mmol) was added dropwise via syringe. The resulting solution was allowed to stir at RT for about 2 h. The pH of the solution was adjusted to pH=about 1 by dropwise addition of 1N HCl solution. The mixture was diluted with DCM and the solution was washed with H₂O (1×10 mL). The organic phase was dried over MgSO₄, filtered and concentrated to give 3-(5-(3-(3-chloro-4-isopropoxyphenyl)-1,2,4-oxadiazol-5-yl)-1H-indol-1-yl)cyclopentanecarboxylic acid (10 mg, 90%) as a colorless oil. LC/MS (Table 1, Method g) Rₜ=3.12 min, m/z 466 (M+H)⁺; ¹H NMR (400 MHz, DMSO) δ 8.47 (d, J=1.7, 1H), 8.10-7.92 (m, 3H), 7.79 (dd, J=11.9, 8.6, 1H), 7.72 (dd, J=7.8, 3.2, 1H), 7.40 (d, J=8.8, 1H), 6.73 (t, J=2.6, 1H), 5.14-5.00 (m, 1H), 4.88-4.78 (m, 1H), 3.13-2.93 (m, 2H), 2.31-2.08 (m, 4H), 2.10-1.94 (m, 2H), 1.39-1.33 (m, 6H).

### Preparation #60

### Preparation 3-(3-chloro-4-isopropoxyphenyl)-5-(4-fluoro-3-((trimethylsilyl)ethynyl)phenyl)-1,2,4-oxadiazole

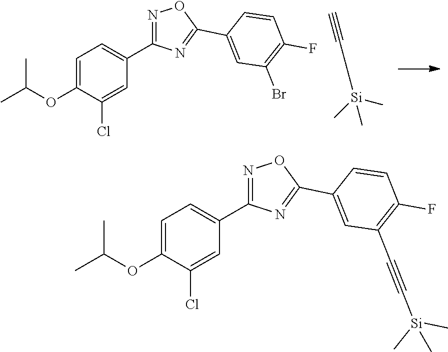

A 25 mL round bottom flask equipped with reflux condenser outfitted with a nitrogen inlet adapter was charged with 5-(3-bromo-4-fluorophenyl)-3-(3-chloro-4-isopropoxyphenyl)-1,2,4-oxadiazole (0.608 g, 1.477 mmol), bis(triphenylphosphine)palladium(II) chloride (0.052 g, 0.074 mmol), copper(I) iodide (0.014 g, 0.074 mmol), evacuated and filled with nitrogen (three cycles), and then THF was added to give a yellow solution. Ethynyltrimethylsilane (0.307 mL, 2.215 mmol) and triethylamine (0.618 mL, 4.43 mmol) were each added sequentially in one portion. The reaction mixture was heated at about 60° C. for about 6 h. The reaction mixture was allowed to cool to RT and concentrated. The sample was deposited onto silica gel and purified via automated silica gel chromatography (2% EtOAc:Heptane; EA-40 g column, 18×150 mm test tubes). The fractions containing product were combined and concentrated to give 3-(3-chloro-4-isopropoxyphenyl)-5-(4-fluoro-3-((trimethylsilyl)ethynyl)phenyl)-1,2,4-oxadiazole (321 mg, 51%) as a solid. LC/MS (Table 1, Method g) Rₜ=2.43 min, m/z 429 (M+H)⁺.

### Preparation #60b

### Preparation of (1R,3S)-3-(4-(3-(3-chloro-4-isopropoxyphenyl)-1,2,4-oxadiazol-5-yl)-2-ethynylphenylamino)cyclopentanecarboxylic acid

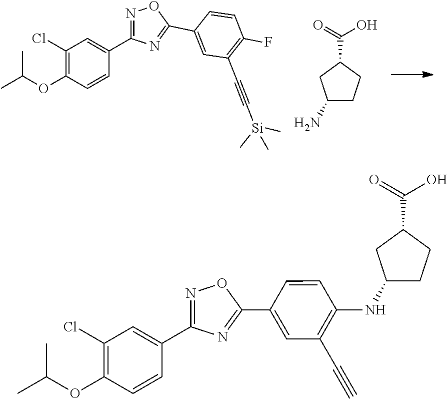

A 20 mL microwave reaction vial equipped with pressure-releasing septa cap was charged with 3-(3-chloro-4-isopropoxyphenyl)-5-(4-fluoro-3-((trimethylsilyl)ethynyl)phenyl)-1,2,4-oxadiazole (0.295 g, 0.688 mmol), (1R,3S)-3-aminocyclopentanecarboxylic acid (0.098 g, 0.756 mmol), and potassium carbonate (0.209 g, 1.513 mmol) in water (0.344 mL) and DMSO (3.09 mL) to give a tan suspension. The reaction mixture was heated at about 170° C. for about 1 h using simultaneous heating while cooling. The reaction mixture was diluted with 10 mL of water and diluted with EtOAc. The solution was washed with 1M HCl solution (3×50 mL) and saturated NaCl solution (1×50 mL). The organic phase was dried over MgSO₄, filtered and concentrated to give a solid that was triturated with MeOH, filtered, and dried open to the air to give 256 mg of a solid that was suspended in DMSO and filtered, washed with 1N HCl solution and dried open to the air to afford (1R,3S)-3-((4-(3-(3-chloro-4-isopropoxyphenyl)-1,2,4-oxadiazol-5-yl)phenyl)(methyl)amino)cyclopentanecarboxylic acid (220 mg, 64%) as a white solid. LC/MS (Table 1, Method g) Rₜ=1.63 min, m/z 466 (M+H)⁺.

### Example #69

### Preparation of (1R,3S)-3-(5-(3-(3-chloro-4-isopropoxyphenyl)-1,2,4-oxadiazol-5-yl)-1H-indol-1-yl)cyclopentanecarboxylic acid

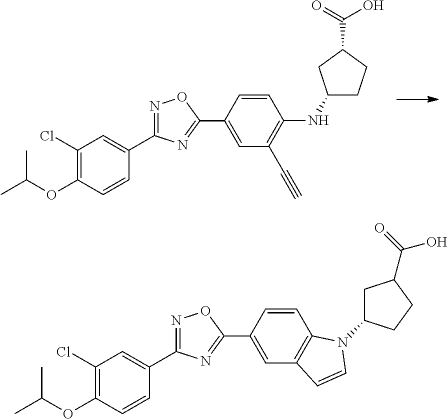

A 50 mL reaction vial equipped with septa cap outfitted with nitrogen inlet needle was charged with (1R,3S)-3-(4-(3-(3-chloro-4-isopropoxyphenyl)-1,2,4-oxadiazol-5-yl)-2-ethynylphenylamino)cyclopentanecarboxylic acid (0.220 g, 0.113 mmol) and gold(I) chloride (2.63 mg, 0.011 mmol) in DCE (1.133 mL) to give a tan solution. The reaction mixture was heated at about 80° C. for about 14 h. The reaction mixture was deposited onto silica gel and loaded onto a silica gel column and eluted with 40%-80% EtOAc:Heptane. The fractions containing product were combined and concentrated to give (1R,3S)-3-(5-(3-(3-chloro-4-isopropoxyphenyl)-1,2,4-oxadiazol-5-yl)-1H-indol-1-yl)cyclopentanecarboxylic acid (5 mg, 2%) as a solid. LC/MS (Table 1, Method g) Rₜ=3.12 min, m/z 466 (M+H)⁺; ¹H NMR (400 MHz, DMSO) δ 8.47 (d, J=1.7, 1H), 8.10-7.92 (m, 3H), 7.79 (dd, J=11.9, 8.6, 1H), 7.72 (dd, J=7.8, 3.2, 1H), 7.40 (d, J=8.8, 1H), 6.73 (t, J=2.6, 1H), 5.14-5.00 (m, 1H), 4.88-4.78 (m, 1H), 3.13-2.93 (m, 2H), 2.31-2.08 (m, 4H), 2.10-1.94 (m, 2H), 1.39-1.33 (m, 6H).

### Preparation #61

### Preparation of (E)-ethyl 4-(3-(3-(3-chloro-4-isopropoxyphenyl)-1,2,4-oxadiazol-5-yl)-2-methylphenoxy)but-2-enoate

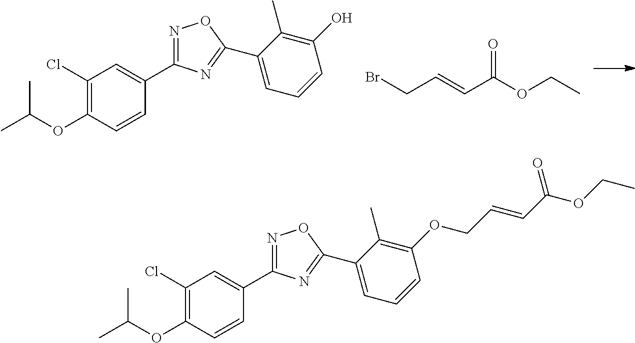

A 50 mL reaction vial equipped with septa cap outfitted with nitrogen inlet needle was charged with 3-(3-(3-chloro-4-isopropoxyphenyl)-1,2,4-oxadiazol-5-yl)-2-methylphenol (0.350 g, 1.015 mmol) and potassium carbonate (0.351 g, 2.54 mmol) in acetone (5.08 mL) to give a colorless suspension. (E)-ethyl 4-bromobut-2-enoate (0.275 mL, 2.030 mmol) was added dropwise via syringe. The resulting suspension was allowed to stir at RT for about 2 days. The reaction mixture was diluted with EtOAc and the solution was washed with 1M HCl solution (1×50 mL), saturated NaHCO₃ solution (1×50 mL), and saturated NaCl solution (1×50 mL). The organic phase was dried over MgSO₄, filtered and concentrated to give 540 mg of a solid. The sample was purified via automated silica gel chromatography (10%-20% EtOAc:Heptane; EA-40 g column, 18×150 mm test tubes). The fractions containing product were combined and concentrated to afford (E)-ethyl 4-(3-(3-(3-chloro-4-isopropoxyphenyl)-1,2,4-oxadiazol-5-yl)-2-methylphenoxy)but-2-enoate (353 mg, 76%) as a solid. LC/MS (Table 1, Method k) Rₜ=1.98 min, m/z 457 (M+H)⁺.

### Preparation #62

### Preparation of Ethyl 4-(3-(3-(3-chloro-4-isopropoxyphenyl)-1,2,4-oxadiazol-5-yl)-2-methylphenoxy)butanoate

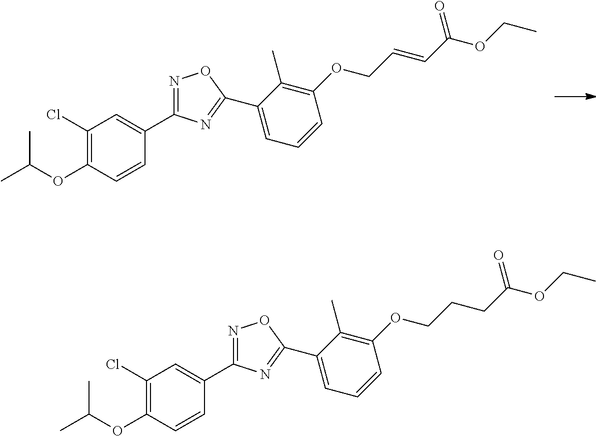

A 100 mL round bottom flask equipped with rubber septum and nitrogen inlet needle was charged with (E)-ethyl 4-(3-(3-(3-chloro-4-isopropoxyphenyl)-1,2,4-oxadiazol-5-yl)-2-methylphenoxy)but-2-enoate (0.336 g, 0.735 mmol), copper(I) chloride (3.64 mg, 0.037 mmol), p-Tol-Binap (0.050 g, 0.074 mmol), sodium tert-butoxide (3.53 mg, 0.037 mmol), evacuated and filled with nitrogen (three cycles), and then toluene (2.94 mL) was added to give a yellow solution. Poly(methylhydrosiloxane) (0.176 mL, 2.94 mmol) was added in one portion. The resulting solution was allowed to stir at RT for about 3 days. EtOH (about 10 mL) was added dropwise. The solution was diluted with EtOAc. The solution was washed with saturated NaHCO₃ solution (1×50 mL), H₂O (1×50 mL), and saturated NaCl solution (1×50 mL). The organic phase was dried over MgSO₄, filtered and concentrated to give 590 mg of a solid. The sample was purified via automated silica gel chromatography (10%-40% EtOAc:Heptane; EA-40 g column, 18×150 mm test tubes). The fractions containing product were combined and concentrated to afford ethyl 4-(3-(3-(3-chloro-4-isopropoxyphenyl)-1,2,4-oxadiazol-5-yl)-2-methylphenoxy)butanoate (178 mg, 53%) as an oil. LC/MS (Table 1, Method g) Rₜ=3.74 min, m/z 459 (M+H)⁺.

### Preparation #63

### Preparation of (E)-4-(3-(3-(3-chloro-4-isopropoxyphenyl)-1,2,4-oxadiazol-5-yl)-2-methylphenoxy)but-2-enoic acid

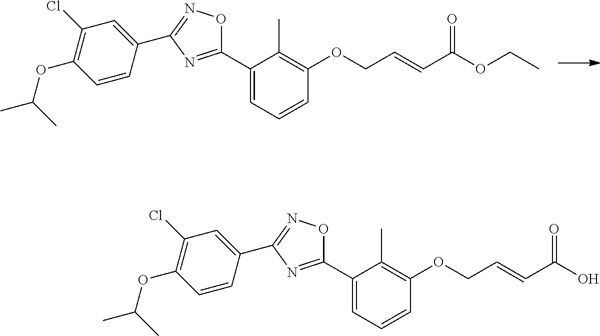

A 25 mL round bottom flask equipped with rubber septum and nitrogen inlet needle was charged with (E)-ethyl 4-(3-(3-(3-chloro-4-isopropoxyphenyl)-1,2,4-oxadiazol-5-yl)-2-methylphenoxy)but-2-enoate (0.255 g, 0.407 mmol) in (1,4?)dioxane (2.037 mL) to give a colorless solution. Sodium hydroxide solution (1.222 mL, 1.222 mmol) was added in one portion. The resulting solution was allowed to stir at RT for about 16 h. The pH was adjusted to pH=about 4 by dropwise addition of acetic acid. The solution was concentrated and triturated with water (1×20 mL). The solid was filtered through a sintered glass funnel, washed with water, and dried in vacuo to give (E)-4-(3-(3-(3-chloro-4-isopropoxyphenyl)-1,2,4-oxadiazol-5-yl)-2-methylphenoxy)but-2-enoic acid (39 mg, 22%) as a white solid.

LC/MS (Table 1, Method g) Rₜ=3.05 min, m/z 429 (M+H)⁺; ¹H NMR (400 MHz, DMSO) δ 8.04 (d, J=2.0, 1H), 8.00 (d, J=8.6, 1H), 7.77 (d, J=7.7, 1H), 7.48-7.33 (m, 4H), 6.74 (d, J=5.8, 1H), 5.07 (dd, J=13.2, 7.1, 1H), 4.86-4.75 (m, 1H), 3.19 (d, J=7.0, 2H), 2.58 (s, 3H), 1.33 (d, J=6.0, 6H).

### Preparation #64

### Preparation of Methyl 4-iodo-2-methoxybenzoate

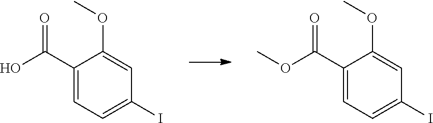

A 250 mL round bottom flask equipped with nitrogen inlet adapter was charged with 4-iodo-2-methoxybenzoic acid (5.0 g, 17.98 mmol) in MeOH (36.0 mL) to give a colorless solution. The reaction mixture was cooled at about 0° C. for about 20 min. Thionyl chloride (3.94 mL, 53.9 mmol) was added slowly via syringe. The resulting solution was allowed to stir at RT for about 18 h. The solution was concentrated and dissolved in EtOAc. The solution was washed with saturated NaHCO₃ solution (1×200 mL) and saturated NaCl solution (1×200 mL). The organic phase was dried over MgSO₄, filtered and concentrated to give methyl 4-iodo-2-methoxybenzoate (5.2 g, 99%) as an oil. LC/MS (Table 1, Method g) Rₜ=2.43 min; ¹H NMR (400 MHz, DMSO) δ 7.47 (s, 1H), 7.38 (s, 2H), 3.81 (s, 3H), 3.75 (s, 3H).

### Preparation #65

### Preparation of Methyl 2-methoxy-4-((1R,4S)-3-oxo-2-azabicyclo[2.2.1]hept-5-en-2-yl) benzoate

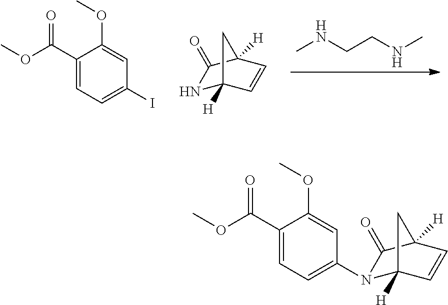

A 50 mL round bottom flask equipped with reflux condenser outfitted with a nitrogen inlet adapter was charged with (1R,4S)-2-azabicyclo[2.2.1]hept-5-en-3-one (1.480 g, 13.56 mmol), copper(I) iodide (0.129 g, 0.678 mmol), potassium phosphate tribasic (5.76 g, 27.1 mmol), evacuated and filled with nitrogen (three cycles), and then toluene (27.1 mL) was added to give a white suspension. Methyl 4-iodo-2-methoxybenzoate (4.95 g, 16.95 mmol) and N¹,N²-dimethylethane-1,2-diamine (0.146 mL, 1.356 mmol) were each added sequentially in one portion. The reaction mixture was heated at about 110° C. for about 18 h. The reaction mixture was filtered and deposited onto silica gel. The crude material was purified via automated silica gel chromatography (10%-50% EtOAc:Heptane; EA-80 g column, 18×150 mm test tubes). The fractions containing product were combined and concentrated to afford methyl 2-methoxy-4-((1R,4S)-3-oxo-2-azabicyclo[2.2.1]hept-5-en-2-yl)benzoate (2.69 g, 74%) as a solid. LC/MS (Table 1, Method g) Rₜ=1.95 min, m/z 274 (M+H)⁺.

### Preparation #66

### Preparation of Methyl 2-methoxy-4-((1S,4R)-3-oxo-2-azabicyclo[2.2.1]heptan-2-yl) benzoate

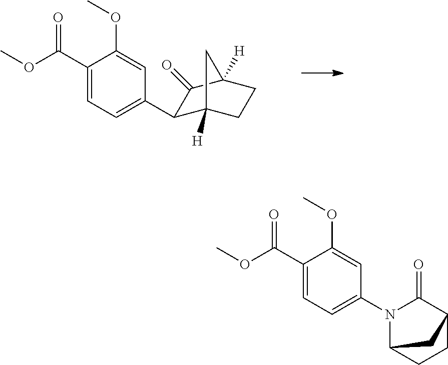

A 250 mL round bottom flask was charged with methyl 2-methoxy-4-((1R,45)-3-oxo-2-azabicyclo[2.2.1]hept-5-en-2-yl)benzoate (2.70 g, 9.88 mmol) in ethyl acetate (99 mL) to give a brown solution. Palladium on carbon (0.210 g, 0.198 mmol) was added in one portion. The resulting suspension was allowed to stir under an atmosphere of hydrogen (balloon) at RT for about 6 h. The suspension was filtered through a pad of Celite®. The filtrate was concentrated to give methyl 2-methoxy-4-((1S,4R)-3-oxo-2-azabicyclo[2.2.1]heptan-2-yl)benzoate (2.73 g, 100%) as a solid. LC/MS (Table 1, Method g) Rₜ=1.91 min, m/z 276 (M+H)⁺.

### Preparation #67

### Preparation of 2-Methoxy-4-((1S,4R)-3-oxo-2-azabicyclo[2.2.1]heptan-2-yl)benzoic acid

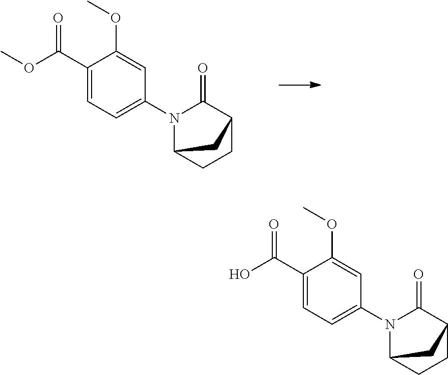

A 25 mL round bottom flask equipped with rubber septum and nitrogen inlet needle was charged with methyl 2-methoxy-4-((1S,4R)-3-oxo-2-azabicyclo[2.2.1]heptan-2-yl)benzoate (0.610 g, 2.216 mmol) in EtOH (8.86 mL) to give a colorless solution. Barium hydroxide solution (8.12 mL, 2.437 mmol) was added dropwise. The resulting suspension was allowed to stir at RT for about 16 h. The pH was adjusted to pH=about 5 by dropwise addition of acetic acid. The solution was concentrated. The solid was triturated with EtOAc (1×25 mL). The solid was removed via filtration through a sintered glass funnel, and the filtrate was dried in vacuo to give 2-methoxy-4-((1S,4R)-3-oxo-2-azabicyclo[2.2.1]heptan-2-yl)benzoic acid (378 mg, 65%) as an oil. LC/MS (Table 1, Method k) Rₜ=1.71 min; ¹H NMR (400 MHz, DMSO) δ 7.67 (t, J=7.8, 1H), 7.42 (dd, J=5.5, 1.9, 1H), 7.14-7.06 (m, 1H), 4.71 (s, 1H), 3.79 (s, 3H), 2.83 (s, 1H), 1.95 (m, 3H), 1.69 (dd, J=17.7, 12.0, 1H), 1.52 (dd, J=17.4, 10.9, 2H).

### Preparation #67a

### Preparation of (1S,4R)-2-(4-(3-(3-chloro-4-isopropoxyphenyl)-1,2,4-oxadiazol-5-yl)-3-methoxyphenyl)-2-azabicyclo[2.2.1]heptan-3-one

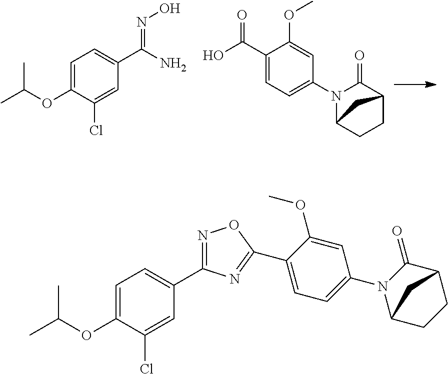

Suspended 2-methoxy-4-((1S,4R)-3-oxo-2-azabicyclo[2.2.1]heptan-2-yl)benzoic acid (0.340 g, 1.301 mmol), HOBt (0.299 g, 1.952 mmol), EDC (0.374 g, 1.952 mmol) in DMF (2.169 mL) under nitrogen. The solution was stirred at RT for about 30 min. (Z)-3-chloro-N′-hydroxy-4-isopropoxybenzimidamide (0.446 g, 1.952 mmol) was added as a solution in DMF (0.434 mL) and heated to about 140° C. for about 4 h. The solution was allowed to cool to RT and precipitated with water. The solids were filtered and then dissolved in EtOAc. The organic phase was washed with 1N HCl solution (1×200 mL), saturated NaHCO₃ solution (1×200 mL), and saturated NaCl solution (1×200 mL), dried over MgSO₄, filtered and concentrated to give (1S,4R)-2-(4-(3-(3-chloro-4-isopropoxyphenyl)-1,2,4-oxadiazol-5-yl)-3-methoxyphenyl)-2-azabicyclo[2.2.1]heptan-3-one (290 mg, 44%) as a solid. LC/MS (Table 1, Method g) Rₜ=3.18 min, m/z 454 (M+H)⁺.

### Example #71

### Preparation of (1R,3S)-3-(4-(3-(3-chloro-4-isopropoxyphenyl)-1,2,4-oxadiazol-5-yl)-3-methoxyphenylamino)cyclopentanecarboxylic acid

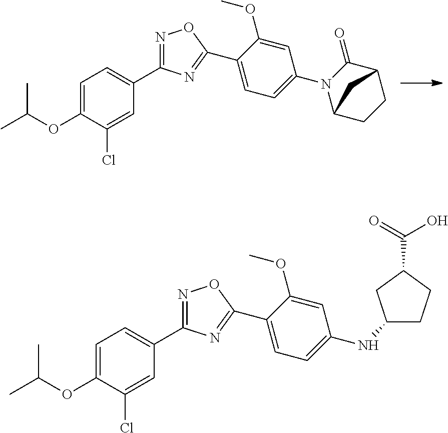

A 75 mL reaction vial equipped with septa cap outfitted with nitrogen inlet needle was charged with (1S,4R)-2-(4-(3-(3-chloro-4-isopropoxyphenyl)-1,2,4-oxadiazol-5-yl)-3-methoxyphenyl)-2-azabicyclo[2.2.1]heptan-3-one (0.290 g, 0.639 mmol) in THF (6.39 mL) to give a colorless solution. Sodium hydroxide solution (6.39 mL, 6.39 mmol) was added dropwise via syringe. The reaction mixture was heated at about 60° C. for about 16 h. The pH was adjusted to pH=about 2 by dropwise addition of 1N HCl solution. The aqueous suspension was extracted with EtOAc (2×50 mL) and washed with saturated NaCl solution (1×50 mL). The organic phase was dried over MgSO₄, filtered and concentrated to give an oil. The sample was purified via automated silica gel chromatography (0%-5% MeOH:CH₂Cl₂;EA-40 g column, 18×150 mm test tubes). The fractions containing product were combined and concentrated to afford 204 mg of a solid. The sample was recrystallized from ether, the solvent was decanted, and the solid was collected and washed with ether, then dried in vacuo to give (1R,3S)-3-(4-(3-(3-chloro-4-isopropoxyphenyl)-1,2,4-oxadiazol-5-yl)-3-methoxyphenylamino)cyclopentanecarboxylic acid (120 mg, 39%) as a solid. LC/MS (Table 1, Method g) Rₜ=2.85 min, m/z 472 (M+H)⁺; ¹H NMR (400 MHz, DMSO) δ 7.98 (d, J=2.1, 1H), 7.92 (dd, J=8.6, 2.1, 1H), 7.82 (d, J=8.7, 1H), 7.34 (d, J=9.0, 1H), 6.79 (d, J=6.9, 1H), 6.33 (dd, J=8.8, 2.0, 1H), 6.29 (d, J=1.8, 1H), 4.85-4.73 (m, 1H), 3.95-3.83 (m, 4H), 2.82-2.71 (m, 1H), 2.40-2.29 (m, 1H), 2.01 (dd, J=12.3, 7.0, 1H), 1.87 (dd, J=15.3, 7.5, 2H), 1.70-1.60 (m, 1H), 1.53 (m, 1H), 1.33 (d, J=6.0, 6H).

### Example #72

### Preparation of (R)-3-(3-(3-(3-chloro-4-isopropoxyphenyl)-1,2,4-oxadiazol-5-yl)-2-methylphenoxy)propane-1,2-diol

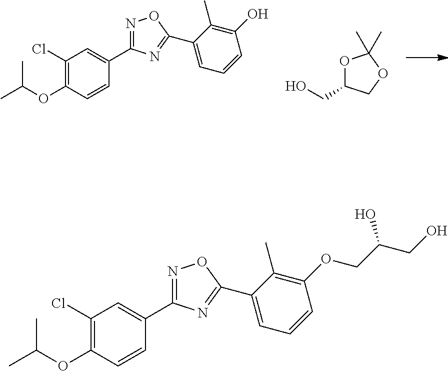

A 50 mL reaction vial equipped with septa cap outfitted with nitrogen inlet needle was charged with 3-(3-(3-chloro-4-isopropoxyphenyl)-1,2,4-oxadiazol-5-yl)-2-methylphenol (0.350 g, 1.015 mmol), (S)-(2,2-dimethyl-1,3-dioxolan-4-yl)methanol (0.139 mL, 1.117 mmol), and PS-triphenylphosphine (0.406 g, 1.218 mmol) in THF (5.08 mL) to give a tan suspension. 4 Å molecular sieves were added in one portion. The resulting suspension was allowed to stir at RT for about 2 days. The reaction mixture was diluted with about 20 mL of ether and HCl (0.508 mL, 6.09 mmol) was added dropwise. The resulting suspension was allowed to stir at RT for about 6 h. The suspension was filtered through a pad of Celite® with the aid of EtOAc. The filtrate was concentrated and the resulting oil was diluted with DCM (5.08 mL). TFA (0.782 mL, 10.15 mmol) was added dropwise. The resulting solution was allowed to stir at RT for about 8 h. The mixture was concentrated and the sample was purified via automated silica gel chromatography (5%-20% EtOAc:Heptane; RS-40 g column, 18×150 mm test tubes). The fractions containing product were combined and concentrated and then dissolved in MeOH. 100 mg of K₂CO₃ was added and the suspension was allowed to stir at RT for about 6 h. The solution was concentrated and triturated with water (2×50 mL), filtered and washed with water, then dried in vacuo to give (R)-3-(3-(3-(3-chloro-4-isopropoxyphenyl)-1,2,4-oxadiazol-5-yl)-2-methylphenoxy)propane-1,2-diol (142 mg, 33%) as a white solid. LC/MS (Table 1, Method g) Rₜ=3.35 min, m/z 457 (M+H)⁺; ¹H NMR (400 MHz, DMSO) δ 8.04 (d, J=2.1, 1H), 7.99 (dd, J=8.6, 2.1, 1H), 7.61 (d, J=7.3, 1H), 7.39 (dd, J=8.5, 5.8, 2H), 7.28 (d, J=8.0, 1H), 4.98 (d, J=5.1, 1H), 4.82 (d, J=6.0, 1H), 4.68 (s, 1H), 4.06 (d, J=4.5, 1H), 3.98 (d, J=5.7, 1H), 3.84 (d, J=5.1, 1H), 3.50 (d, J=2.7, 2H), 2.54 (s, 3H), 1.34 (d, J=6.0, 6H).

### Preparation #68

### Preparation of diethyl(1R,3R)-3-(4-(3-(3-chloro-4-isopropoxyphenyl)-1,2,4-oxadiazol-5-yl)phenylamino)cyclopentylphosphonate, diethyl(1S,3R)-3-(4-(3-(3-chloro-4-isopropoxyphenyl)-1,2,4-oxadiazol-5-yl)phenylamino)cyclopentylphosphonate, diethyl(1R,3S)-3-(4-(3-(3-chloro-4-isopropoxyphenyl)-1,2,4-oxadiazol-5-yl)phenylamino)cyclopentylphosphonate and diethyl(1S,3S)-3-(4-(3-(3-chloro-4-isopropoxyphenyl)-1,2,4-oxadiazol-5-yl)phenylamino)cyclopentylphosphonate

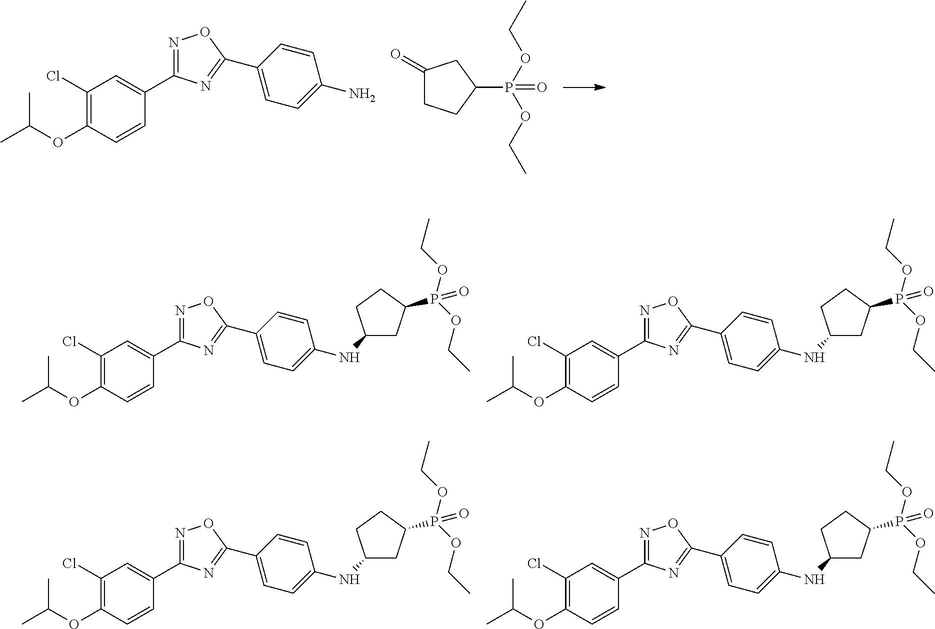

A solution of 4-(3-(3-chloro-4-isopropoxyphenyl)-1,2,4-oxadiazol-5-yl)aniline (0.430 g, 1.30 mmol, 4-(3-(3-chloro-4-isopropoxyphenyl)-1,2,4-oxadiazol-5-yl)aniline was prepared from 3-chloro-4-isopropoxybenzonitrile and 4-aminobenzoic acid by following General Procedures C and D.), diethyl 3-oxocyclopentylphosphonate (0.861 g, 3.91 mmol, prepared according to Journal of Medicinal Chemistry (1986), 29(10), 1988-95.), acetic acid (1.50 mL, 26.1 mmol), 4 Å molecular sieves (1.5 g) in DCM (6.5 mL) and MeOH (6.52 mL) was heated at about 40° C. for about 6 h, then NaCNBH₃ (0.041 g, 0.65 mmol) was added. The reaction mixture was stirred at RT for about 16 h. Water (20 mL) was added to the reaction mixture. After filtration, the filtrate was concentrated under reduced pressure and purified on silica gel (80 g) using (1-5% MeOH in DCM) followed by chiral chromatography (The gradient was 0-40% A in 19 min (20 mL/min flow rate). Mobile phase A was 200 proof ethanol, mobile phase B was HPLC grade heptane with 0.1% diethylamine added. The column used for the chromatography was a Daicel OD-H, 20×250 mm column (5 μm particles). Detection methods were UV (330 nm) detection as well as optical rotation) to give diethyl(1R,3R)-3-(4-(3-(3-chloro-4-isopropoxyphenyl)-1,2,4-oxadiazol-5-yl)phenylamino)cyclopentylphosphonate [Rₜ 12.3 min, optical rotation=positive] (0.060 g, 9%): LC/MS (Table 1, Method b) Rₜ=2.87 min; MS m/z: 534 (M+H)⁺; diethyl(1S,3R)-3-(4-(3-(3-chloro-4-isopropoxyphenyl)-1,2,4-oxadiazol-5-yl)phenylamino)cyclopentylphosphonate[Rₜ 13.4 min, or =negative] (0.065 g, 9%): LC/MS (Table 1, Method b) Rₜ=2.82 min; MS m/z: 534 (M+H)⁺; diethyl(1R,3S)-3-(4-(3-(3-chloro-4-isopropoxyphenyl)-1,2,4-oxadiazol-5-yl)phenylamino)cyclopentylphosphonate [Rₜ 15.4 min, optical rotation=positive] (0.149 g, 21%): LC/MS (Table 1, Method b) Rₜ=2.87 min; MS m/z: 534 (M+H)⁺; diethyl(1S,3S)-3-(4-(3-(3-chloro-4-isopropoxyphenyl)-1,2,4-oxadiazol-5-yl)phenylamino)cyclopentylphosphonate[Table 3, Method 9, Rₜ 13.4 min, or=negative] (0.052 g, 7%): LC/MS (Table 1, Method b) Rₜ=2.82 min; MS m/z: 534 (M+H)⁺.

### Example #73

### Preparation of N-(4-(3-(3-chloro-4-isopropoxyphenyl)-1,2,4-oxadiazol-5-yl)phenyl) isonicotinamide

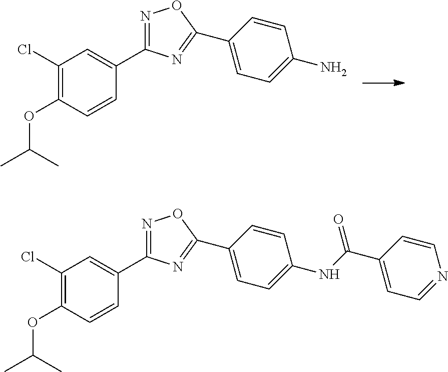

4-(3-(3-chloro-4-isopropoxyphenyl)-1,2,4-oxadiazol-5-yl)aniline was prepared from 3-chloro-4-isopropoxybenzonitrile and 4-aminobenzoic acid by following General Procedures C and D. Isonicotinic acid (0.112 g, 0.910 mmol) was dissolved in thionyl chloride (5 mL, 68.5 mmol) to give a colorless solution. DMF (3 drops) was added, the reaction mixture was heated at about 70° C. for about 16 h. Cooled down, the solvent was removed to afford roughly 0.2 g pale yellow solid. 4-(3-(3-chloro-4-isopropoxyphenyl)-1,2,4-oxadiazol-5-yl)aniline (0.15 g, 0.455 mmol) was dissolved in DCM (5 mL) to give a colorless solution, the reaction mixture was cooled to about 0-5° C. in an ice bath. The cloudy suspension of the 0.2 g pale yellow solid in DCM (5 mL) was added dropwise to the solution. The ice-bath was removed, the reaction mixture was stirred at RT overnight. The reaction mixture was concentrated in vacuo, the yellow residue was triturated by methanol (3 mL) and DMSO (3 mL), the resulting suspension was filtered, the solid was washed by methanol (2×5 mL). Dried in oven overnight to afford 0.18 g solid, to which was added DMSO (2 mL) and methanol (2 mL), heated at about 80° C. for about 30 min. The solution was cooled down, the mixture was filtered, the solid was collected and washed by methanol (5 mL) and water (2×5 mL), and dried to afford N-(4-(3-(3-chloro-4-isopropoxyphenyl)-1,2,4-oxadiazol-5-yl)phenyl)isonicotinamide (0.12 g, 0.276 mmol, 60.7% yield) as yellow powder: LC/MS (Table 1, Method a) Rₜ=4.39 min.; MS m/z: 435.19 (M+H)⁺. 1H NMR (400 MHz, DMSO-d6) δ ppm 11.17 (s, 1H), 8.96 (dd, J=4.84, 1.50 Hz, 2H), 8.26-8.20 (m, 2H), 8.17 (dd, J=4.82, 1.54 Hz, 2H), 8.14-8.09 (m, 2H), 8.06 (d, J=2.11 Hz, 1H), 8.00 (dd, J=8.63, 2.13 Hz, 1H), 7.40 (d, J=8.88 Hz, 1H), 4.91-4.75 (m, 1H), 1.36 (d, J=6.02 Hz, 6H).

### Example #74

### Preparation of 3-amino-1-(4-(3-(3-chloro-4-isopropoxyphenyl)-1,2,4-oxadiazol-5-yl)benzyl)pyrrolidine-3-carboxylic acid

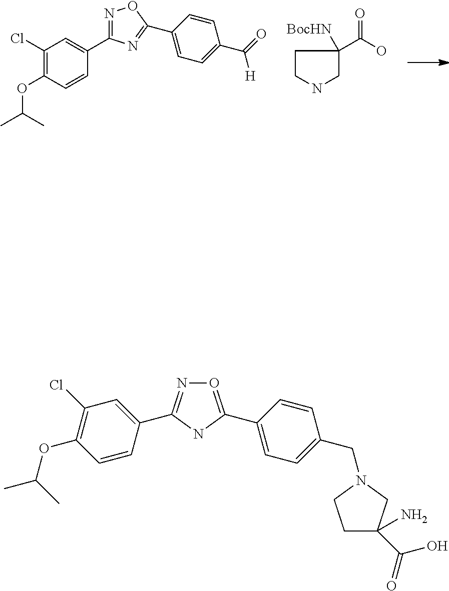

3-Amino-1-(4-(3-(3-chloro-4-isopropoxyphenyl)-1,2,4-oxadiazol-5-yl)benzyl)pyrrolidine-3-carboxylic acid was made by General Procedures H and K. LC/MS (Table 1, Method g) Rₜ=1.94 min, m/z 457.14 (M+H)⁺. 1H NMR (400 MHz, DMSO-d6) δ ppm 8.12 (d, J=8.29 Hz, 2H), 8.05 (d, J=2.11 Hz, 1H), 7.99 (dd, J=8.64, 2.12 Hz, 1H), 7.65 (d, J=8.28 Hz, 2H), 7.38 (d, J=8.97 Hz, 1H), 4.81 (dt, J=6.0, 12.0 Hz, 1H), 3.73 (dd, J=24.40, 10.0 Hz, 2H), 2.96-2.90 (m, 1H), 2.75 (dd, J=34.80, 10.0 Hz, 2H), 2.37-2.27 (m, 2H), 1.80-1.72 (m, 1H), 1.34 (d, J=6.02 Hz, 6H).

### Preparation #69

### Preparation of (1R,3S)-methyl 3-(dibenzylamino)cyclopentanecarboxylate

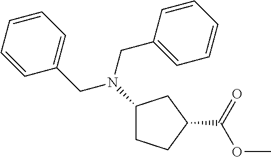

### Step 1

### (1R,3S)-Methyl 3-aminocyclopentanecarboxylate Hydrochloride

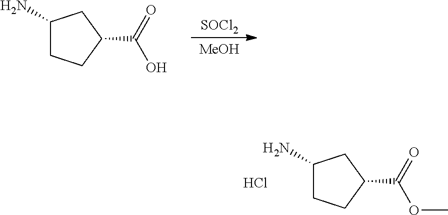

A solution of (1R,3S)-3-aminocyclopentanecarboxylic acid (0.115 g, 0.890 mmol) (PepTech) in MeOH (1.5 mL) was cooled to about 0° C. in an ice bath. Thionyl chloride (0.130 mL, 1.781 mmol) was added dropwise. The reaction mixture was stirred at about 0° C. for about 2 h, then the reaction mixture was heated at about 65° C. for about 18 h until TLC in (3:1:1 n-BuOH/AcOH/water) showed (Ninhydrin/EtOH visualization) less polar spot forming (Product's Rf=0.428, SM's Rf=0.345). The reaction mixture was cooled down and concentrated to afford (1R,3S)-Methyl 3-aminocyclopentanecarboxylate hydrochloride (0.16 g, 0.89 mmol, 100% yield) as a pale green solid.

### Step 2

### (1R,3S)-Methyl 3-(dibenzylamino)cyclopentane carboxylate

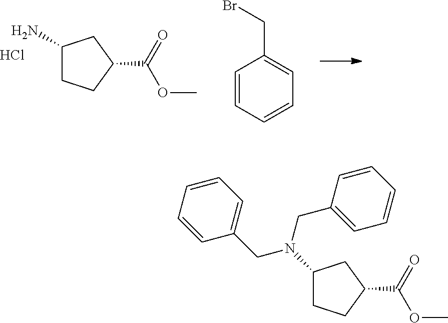

A solution of sodium carbonate (0.296 g, 2.79 mmol) in water (0.840 mL) was added DCM (1.680 mL). (1R,3S)-Methyl 3-aminocyclopentanecarboxylate hydrochloric acid (0.125 g, 0.698 mmol) was added, then benzyl bromide (0.170 mL, 1.431 mmol) was added. The reaction mixture was heated at about 40° C. for about 7 h. The reaction mixture was cooled down, partitioned between EtOAc (30 mL) and water (30 mL), the organic layer was washed by brine (20 mL), dried over sodium sulfate, filtered and concentrated to afford 237 mg pale yellow oil, which was purified via Analogix® (0-25% EtOAc/Heptane over 20 min; 12 g Redi-Sep® silica gel column) to afford (1R,3S)-methyl 3-(dibenzylamino)cyclopentanecarboxylate (0.195 g, 0.603 mmol, 86% yield) as colorless oil.

LC/MS (Table 1, Method g) Rₜ=2.89 min, m/z 324.19 (M+H)⁺.

### Preparation #70

### Preparation of (3S)-methyl 3-amino-1-fluorocyclopentanecarboxylate

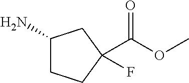

### Step 1

### (3S)-Methyl 3-(dibenzylamino)-1-fluorocyclopentanecarboxylate

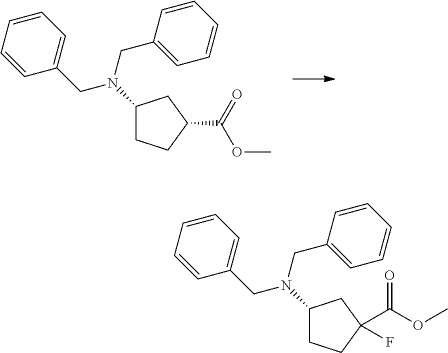

A solution of (1R,3S)-methyl 3-(dibenzylamino)cyclopentanecarboxylate (0.139 g, 0.430 mmol) in THF (1.433 mL) was cooled to about −78° C. in a dry ice/acetone bath. Potassium hexamethyldisiazide (0.945 mL, 0.473 mmol) (0.5 M in toluene) was added dropwise over about 5 min, the reaction mixture was stirred at about −78° C. for about 50 min. A solution of N-fluoro-N-(phenylsulfonyl)benzenesulfonamide (0.163 g, 0.516 mmol) in THF (0.716 mL) was added dropwise over about 1 min, then the cooling bath was removed, the reaction mixture was warmed up to RT. Hydrochloric acid (0.1 M, 4.30 mL, 0.430 mmol) was added to quench the reaction. The mixture was partitioned between water (30 mL) and ether (30 mL), the aqueous layer was extracted by ether (2×15 mL), the combined ether layer was washed by brine (20 mL), dried over sodium sulfate, filtered and concentrated to afford 198 mg yellow oil, which was purified via Analogix® (0-30% EtOAc/Heptane over 20 min; 12 g Redi-Sep® silica gel column) to afford (3S)-methyl 3-(dibenzylamino)-1-fluorocyclopentanecarboxylate (0.098 g, 0.287 mmol, 66.8% yield) as colorless oil. LC/MS (Table 1, Method g) Rₜ=3.11 min, m/z 342.15 (M+H)⁺.

### Step 2

### (3S)-Methyl 3-amino-1-fluorocyclopentanecarboxylate

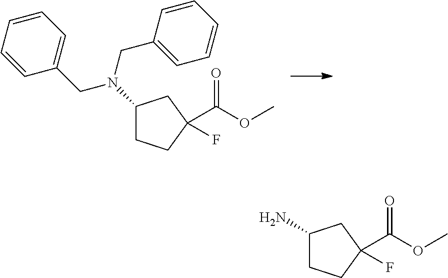

(3S)-Methyl 3-amino-1-fluorocyclopentanecarboxylate was made by General Procedure N and it used in the next step without further purification.

### Preparation #71

### Preparation of (3S)-methyl 3-amino-1-methylcyclopentanecarboxylate

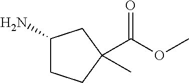

### Step 1

### (3S)-Methyl 3-(dibenzylamino)-1-methylcyclopentanecarboxylate

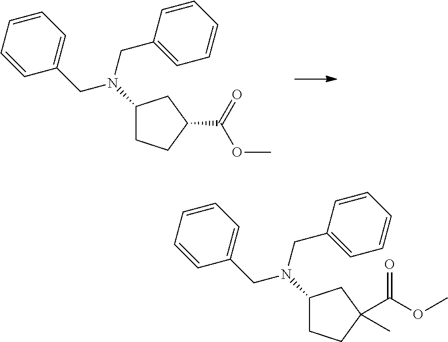

A solution of diisopropylamine (0.521 mL, 3.65 mmol) in THF (2.77 mL) was cooled to about 0° C. in an ice bath, n-butyllithium (2.075 mL, 3.32 mmol) (1.6 M in hexane) was added dropwise over about 2 min. The solution was stirred at about 0° C. for about 15 min. Then the reaction mixture was cooled to about −78° C. in a dry ice/acetone bath. A solution of (1R,3S)-methyl 3-(dibenzylamino)cyclopentanecarboxylate (0.358 g, 1.107 mmol) in THF (1.384 mL) was added dropwise over about 8 min. It was stirred at about −78° C. for about 80 min. A colorless solution of methyl iodide (0.415 mL, 6.64 mmol) in THF (1.384 mL) was added dropwise over about 13 min. The reaction mixture was stirred at about −78° C. for about 24 min. The cooling bath was removed, the reaction mixture was warmed to RT and stirred at RT overnight. The crude mixture was poured into saturated ammonium chloride (25 mL), rinsed by some water, extracted by EtOAc (50 mL), the organic layer was washed by water (20 mL) and brine (20 mL), dried over sodium sulfate, filtered and concentrated to afford 390 mg orange oil, which was purified via Analogix® (0-25% EtOAc/Heptane over 20 min; 40 g Redi-Sep® silica gel column) to afford (3S)-methyl 3-(dibenzylamino)-1-methylcyclopentanecarboxylate (0.34 g, 1.008 mmol, 91% yield) as orange oil. LC/MS (Table 1, Method g) Rₜ=3.11 min, m/z 338.19 (M+H)⁺.

### Step 2

### (3S)-Methyl 3-amino-1-methylcyclopentanecarboxylate

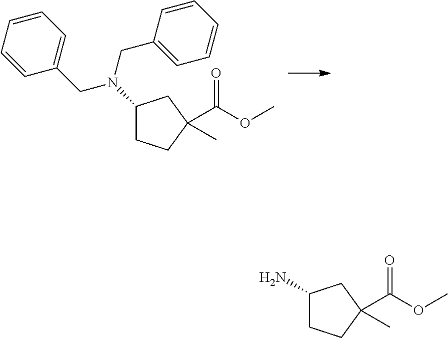

(3S)-Methyl 3-amino-1-methylcyclopentanecarboxylate was made using General Procedure N. TLC Rf=0.545 in 3:1:1 n-BuOH/AcOH/water (KMnO₄ visualization).

### Preparation #72

### Preparation of (3S)-methyl 3-amino-1-hydroxycyclopentanecarboxylate

### Step 1

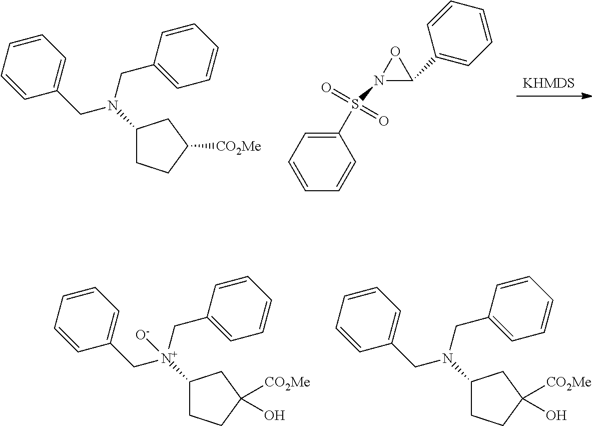

A solution of KHMDS (0.557 mL, 0.278 mmol) in THF (2.441 mL) was cooled to about −78° C. in a dry ice/acetone bath. A solution of (1R,3S)-methyl 3-(dibenzylamino)cyclopentanecarboxylate (0.06 g, 0.186 mmol) in THF (1.953 mL) was added dropwise over about 3 min. The reaction mixture turned into pink orange gradually in the addition process. The solution was stirred at about −78° C. for about 70 min. A solution of (R)-3-phenyl-2-(phenylsulfonyl)-1,2-oxaziridine (0.073 g, 0.278 mmol) (Organic Syntheses, 1988, 66, 203) in THF (4.88 mL) was added dropwise over about 3 min. The solution was stirred at about −78° C. for about 70 min, then saturated NH₄Cl (0.5 mL) was added to quench the reaction and the reaction mixture was warmed to RT. The reaction mixture was concentrated in vacuo, to the aqueous residue was added EtOAc (15 mL) and water (5 mL), the aqueous layer was removed, the organic layer was washed by water (10 mL) and brine (10 mL), dried over sodium sulfate, filtered and concentrated to afford 140 mg yellow solid, which was purified via Analogix® (0-50% EtOAc/Heptane over 20 min; 40 g Redi-Sep® silica gel column) to afford 20 mg yellow oil. LC/MS showed a mixture of (3S)-methyl 3-amino-1-hydroxycyclopentanecarboxylate and N-oxide, LC/MS (Table 1, Method h) Rₜ=2.09; 2.57 min, m/z 340.2; 356.2 (M+H)⁺. (3S)-methyl 3-amino-1-hydroxycyclopentanecarboxylate is used without further purification in next step.

### Step 2

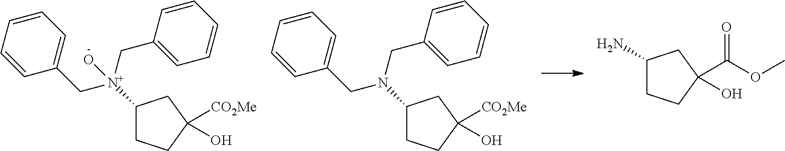

(S)-3-Amino-1-hydroxy-cyclopentanecarboxylic acid methyl ester was made using General Procedure E. LC/MS (Table 1, Method h) Rₜ=0.46 min, m/z 160.09 (M+H)⁺.

### Example #75

### Preparation of (3S)-3-(4-(3-(3-chloro-4-isopropoxyphenyl)-1,2,4-oxadiazol-5-yl)phenylamino)-1-hydroxycyclopentanecarboxylic acid

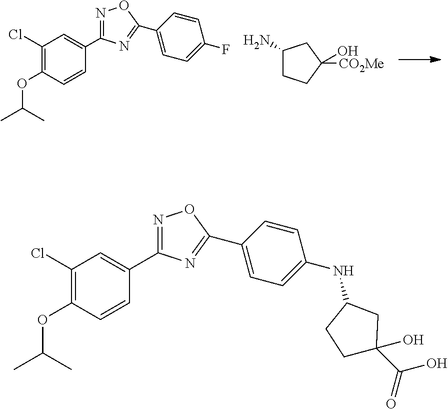

(3S)-3-(4-(3-(3-chloro-4-isopropoxyphenyl)-1,2,4-oxadiazol-5-yl)phenylamino)-1-hydroxycyclopentanecarboxylic acid was made using General Procedures P and Q LC/MS (Table 1, Method g) Rₜ=2.39 min, m/z 458.09 (M+H)⁺. 1H NMR (400 MHz, DMSO-d6) δ ppm 8.02 (d, J=1.9, 1H), 7.96 (dd, J=8.7, 2.0, 1H), 7.87 (dd, J=8.9, 2.9, 2H), 7.36 (d, J=8.8, 1H), 6.93-6.86 (m, 1H), 6.82 (d, J=6.9, 1H), 6.72 (d, J=8.8, 2H), 4.88-4.74 (m, 1H), 4.11-3.87 (m, 2H), 2.59-2.52 (m, 1H), 2.31-2.08 (m, 2H), 2.05-1.89 (m, 1H), 1.89-1.67 (m, 2H), 1.68-1.50 (m, 1H), 1.35 (d, J=6.0, 6H).

### Preparation #73

### Preparation of 3-(3-chloro-4-isopropoxyphenyl)-5-(4-(chloromethyl)phenyl)-1,2,4-oxadiazole

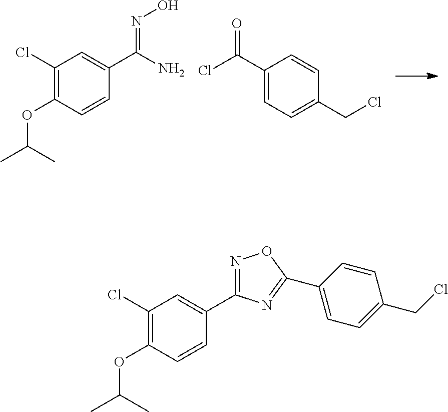

3-(3-chloro-4-isopropoxyphenyl)-5-(4-(chloromethyl)phenyl)-1,2,4-oxadiazole was made using General Procedure E. LC/MS (Table 1, Method h) Rₜ=3.15 min, m/z 365.09 (M+H)⁺. 1H NMR (400 MHz, DMSO-d6) δ ppm 8.25-8.17 (m, 2H), 8.07 (d, J=2.12 Hz, 1H), 8.01 (dd, J=8.65, 2.13 Hz, 1H), 7.73 (d, J=8.26 Hz, 2H), 7.40 (d, J=8.87 Hz, 1H), 4.90 (s, 2H), 4.83 ((dt, J=6.0, 12.0 Hz, 1H), 1.35 (d, J=6.02 Hz, 6H).

### Preparation #74

### Preparation of (4-(3-(3-chloro-4-isopropoxyphenyl)-1,2,4-oxadiazol-5-yl)benzyl)triphenylphosphonium chloride

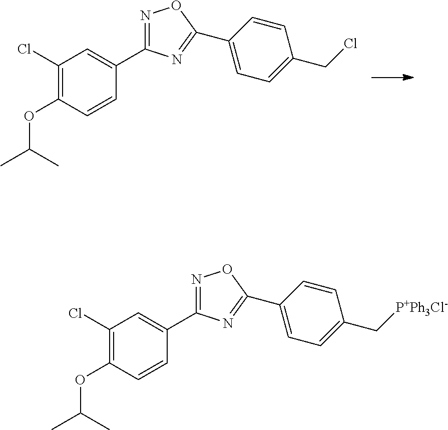

3-(3-chloro-4-isopropoxyphenyl)-5-(4-(chloromethyl)phenyl)-1,2,4-oxadiazole (1.653 g, 4.55 mmol) and triphenylphosphine (1.790 g, 6.83 mmol) in Xylene (30.3 mL) were added to give a cloudy colorless solution. The solution was heated at about 140° C. for about 18 h. The solution was cooled, the white suspension was filtered, washed by heptane (3×8 mL), dried in vacuo (at about 60° C.) for about 3 h to afford (4-(3-(3-chloro-4-isopropoxyphenyl)-1,2,4-oxadiazol-5-yl)benzyl)triphenylphosphonium chloride (1.812 g, 2.90 mmol, 63.7% yield) as white solid. LC/MS (Table 1, Method g) Rₜ=2.89 min, m/z 589.11 (M+H)⁺. 1H NMR (400 MHz, DMSO-d6) δ ppm 8.06-8.02 (m, 3H), 8.00-7.86 (m, 4H), 7.78-7.67 (m, 12H), 7.38 (d, J=8.97 Hz, 1H), 7.23 (dd, J=8.44, 2.35 Hz, 2H), 5.32 (d, J=16.30 Hz, 2H), 4.81 (dt, J=6.0, 12.0 Hz, 1H), 1.33 (d, J=6.02 Hz, 6H).

### Example #76

### Preparation of (1R,3S)-3-(4-(5-(3-chloro-4-isopropoxyphenyl)-1,2,4-oxadiazol-3-yl)phenylamino)cyclopentanecarboxylic acid

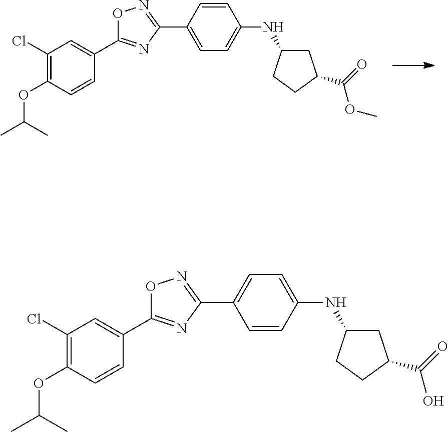

(1R,3S)-3-(4-(5-(3-chloro-4-isopropoxyphenyl)-1,2,4-oxadiazol-3-yl)phenylamino)cyclopentanecarboxylic acid was prepared using General Procedure Q

LC/MS (Table 1, Method g) Rₜ=2.94 min, m/z 442.27 (M+H)⁺. 1H NMR (400 MHz, DMSO-d6) δ ppm 12.10 (s, 1H), 8.14 (d, J=2.09 Hz, 1H), 8.07 (dd, J=8.67, 2.16 Hz, 1H), 7.78 (d, J=8.68 Hz, 2H), 7.42 (d, J=8.90 Hz, 1H), 6.69 (d, J=8.77 Hz, 2H), 6.40 (d, J=6.66 Hz, 1H), 4.87 (dt, J=6.0, 12.0 Hz, 1H), 3.86-3.77 (m, 1H), 2.83-2.72 (m, 1H), 2.37-2.30 (m, 1H), 2.05-1.96 (m, 1H), 1.93-1.82 (m, 2H), 1.69-1.61 (m, 1H), 1.57-1.49 (m, 1H), 1.36 (d, J=6.01 Hz, 6H).

### Example #77

### Preparation of 1-Amino-3-(4-(3-(3-chloro-4-isopropoxyphenyl)-1,2,4-oxadiazol-5-yl)phenoxy)cyclopentanecarboxylic acid

### Step 1

### Benzyl 4-(3-(tert-butoxycarbonylamino)-3-(ethoxycarbonyl)cyclopentyloxy)benzoate

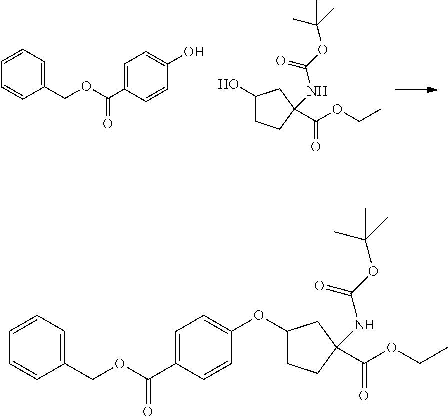

Benzyl 4-(3-(tert-butoxycarbonylamino)-3-(ethoxycarbonyl)cyclopentyloxy)benzoate was prepared using General Procedure R. LC/MS (Table 1, Method g) Rₜ=3.05 min, m/z 484.14 (M+H)⁺.

### Step 2

### 4-(3-(tert-butoxycarbonylamino)-3-(ethoxycarbonyl)cyclopentyloxy)benzoic acid

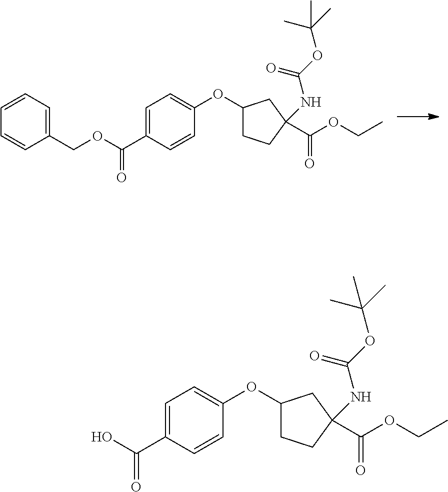

4-(3-(tert-butoxycarbonylamino)-3-(ethoxycarbonyl)cyclopentyloxy)benzoic acid was prepared using General Procedure N. LC/MS (Table 1, Method g) Rₜ=2.16 min, m/z 394.15 (M+H)⁺.

### Step 3

### Ethyl 1-(tert-butoxycarbonylamino)-3-(4-(3-(3-chloro-4-isopropoxyphenyl)-1,2,4-oxadiazol-5-yl)phenoxy)cyclopentanecarboxylate

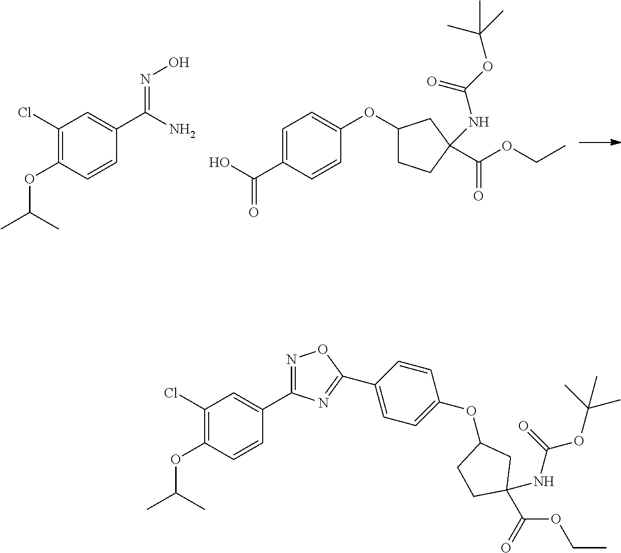

Ethyl 1-(tert-butoxycarbonylamino)-3-(4-(3-(3-chloro-4-isopropoxyphenyl)-1,2,4-oxadiazol-5-yl)phenoxy)cyclopentanecarboxylate was prepared using General Procedure D. LC/MS (Table 1, Method g) Rₜ=3.78 min, m/z 586.22 (M+H)⁺.

### Step 4

### Ethyl 1-amino-3-(4-(3-(3-chloro-4-isopropoxyphenyl)-1,2,4-oxadiazol-5-yl)phenoxy)cyclopentanecarboxylate

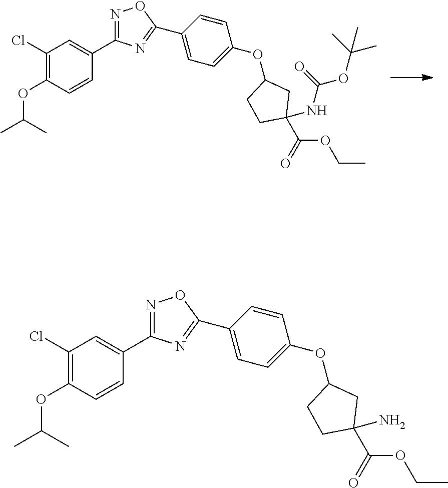

Ethyl 1-amino-3-(4-(3-(3-chloro-4-isopropoxyphenyl)-1,2,4-oxadiazol-5-yl)phenoxy)cyclopentanecarboxylate was prepared using General Procedure K. LC/MS (Table 1, Method g) Rₜ=2.57 min, m/z 486.10 (M+H)⁺.

### Step 5

### 1-Amino-3-{4-[3-(3-chloro-4-isopropoxy-phenyl)-[1,2,4]oxadiazol-5-yl]-phenoxy}-cyclopentanecarboxylic acid

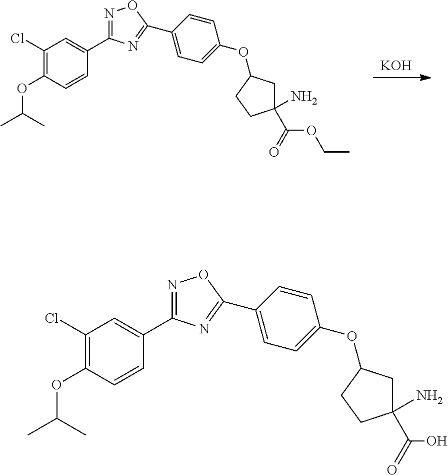

1-Amino-3-{-4-[3-(3-chloro-4-isopropoxy-phenyl)-[1,2,4]oxadiazol-5-yl]-phenoxy}-cyclopentanecarboxylic acid was prepared using General Procedure P. LC/MS (Table 1, Method g) Rₜ=2.08 min, m/z 458.11 (M+H)⁺. ¹H NMR (400 MHz, DMSO-d6) δ ppm 8.70-8.20 (brs, 2H), 8.13 (d, J=8.9, 2H), 8.04 (d, J=2.1, 1H), 7.98 (dd, J=8.6, 2.1, 1H), 7.38 (d, J=9.0, 1H), 7.21 (d, J=8.9, 2H), 5.16-5.09 (m, 1H), 4.82 (hept, J=6.1, 1H), 2.77 (dd, J=14.7, 6.4, 1H), 2.44-2.19 (m, 2H), 2.20-2.02 (m, 3H), 1.35 (d, J=6.0, 6H).

### Example #78

### Preparation of 1-Amino-3-(3-chloro-4-(5-(5-chloro-6-isopropoxypyridin-3-yl)-1,2,4-oxadiazol-3-yl)phenoxy)cyclopentanecarboxylic acid

### Step 1

### Ethyl 1-(tert-butoxycarbonylamino)-3-(3-chloro-4-(5-(5-chloro-6-isopropoxypyridin-3-yl)-1,2,4-oxadiazol-3-yl)phenoxy)cyclopentanecarboxylate

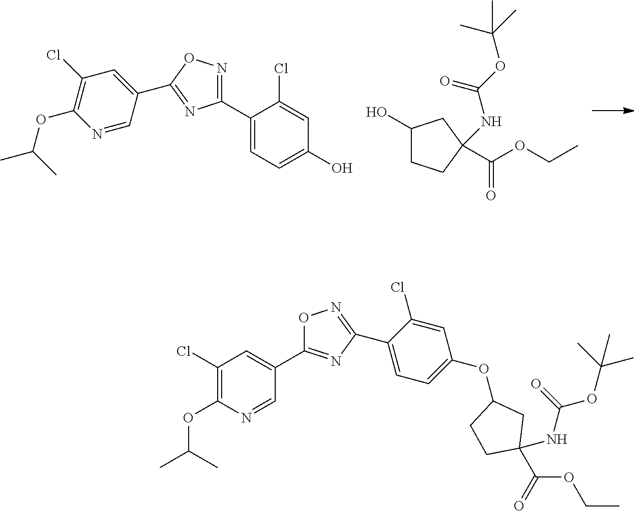

Ethyl 1-(tert-butoxycarbonylamino)-3-(3-chloro-4-(5-(5-chloro-6-isopropoxypyridin-3-yl)-1,2,4-oxadiazol-3-yl)phenoxy)cyclopentanecarboxylate was prepared using General Procedure M. LC/MS (Table 1, Method i) Rₜ=2.10 min, m/z 621.20 (M+H)⁺.

### Step 2

### Ethyl 1-amino-3-(3-chloro-4-(5-(5-chloro-6-isopropoxypyridin-3-yl)-1,2,4-oxadiazol-3-yl)phenoxy)cyclopentanecarboxylate

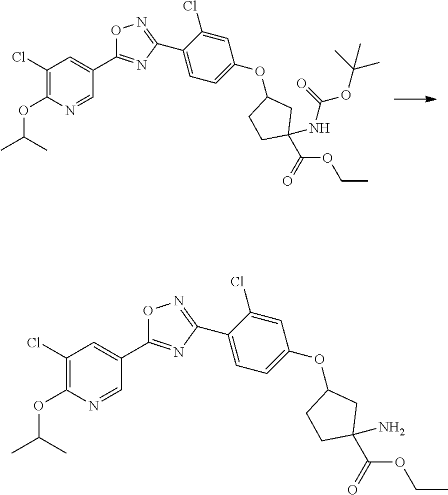

Ethyl 1-amino-3-(3-chloro-4-(5-(5-chloro-6-isopropoxypyridin-3-yl)-1,2,4-oxadiazol-3-yl)phenoxy)cyclopentanecarboxylate was prepared using General Procedure K. LC/MS (Table 1, Method g) Rₜ=2.90 min, m/z 521.09 (M+H)⁺.

### Step 3

### 1-Amino-3-(3-chloro-4-(5-(5-chloro-6-isopropoxypyridin-3-yl)-1,2,4-oxadiazol-3-yl)phenoxy)cyclopentanecarboxylic acid

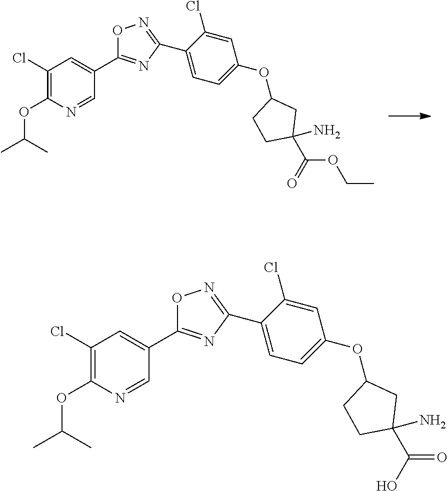

1-Amino-3-(3-chloro-4-(5-(5-chloro-6-isopropoxypyridin-3-yl)-1,2,4-oxadiazol-3-yl)phenoxy)cyclopentanecarboxylic acid was prepared using General Procedure P. LC/MS (Table 1, Method g) Rₜ=2.14 min, m/z 493.10 (M+H)⁺. 1HNMR (400 MHz, DMSO-d6) δ ppm 8.91 (d, J=2.1, 1H), 8.53 (d, J=2.1, 1H), 7.97 (d, J=8.7, 1H), 7.89-7.60 (brs, 2H), 7.25 (d, J=2.4, 1H), 7.15 (dd, J=8.8, 2.4, 1H), 5.51-5.39 (m, 1H), 5.14-5.05 (m, 1H), 2.65 (dd, J=14.1, 6.1, 1H), 2.33-2.18 (m, 1H), 2.18-2.05 (m, 1H), 1.99-1.80 (m, 3H), 1.39 (d, J=6.2, 6H).

### Example #79

### Preparation of (1R,3S)-3-(4-(5-(3-chloro-4-isopropoxyphenyl)-1,2,4-oxadiazol-3-yl)-3-methylphenylamino)cyclopentanecarboxylic acid

### Step 1

### 2-Methyl-4-((1R,4S)-3-oxo-2-azabicyclo[2.2.1]hept-5-en-2-yl)benzonitrile

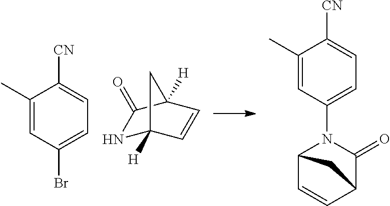

4-Bromo-2-methylbenzonitrile (10 g, 51.0 mmol) and (1R,4S)-2-azabicyclo[2.2.1]hept-5-en-3-one (4.64 g, 42.5 mmol) in toluene (85 mL) were added to give pale yellow suspension. N¹,N²-dimethylethane-1,2-diamine (0.458 mL, 4.25 mmol) was added, the grinded potassium phosphate tribasic (18.05 g, 85 mmol) was added. The reaction mixture was evacuated and refilled with nitrogen for three times. Copper(I) iodide (0.405 g, 2.125 mmol) was added. The reaction mixture was evacuated and refilled with nitrogen for three times, a condenser was put on under nitrogen. The resulting green suspension was heated at about 130° C. for about 16 h. The reaction mixture was cooled and diluted with EtOAc (250 mL), washed with water (3×100 mL), HCl (1 N, 2×100 mL), and water (100 mL), the organic layer was filtered through Celite®, and concentrated to afford 10.7 g yellow solid, which was purified via Analogix® (10%-50% EtOAc/Heptane over 30 min; 120 g Redi-Sep® silica gel column) to afford 2-methyl-4-((1R,4S)-3-oxo-2-azabicyclo[2.2.1]hept-5-en-2-yl)benzonitrile (4.12 g, 18.37 mmol, 43.2% yield) as white solid. LC/MS (Table 1, Method g) Rₜ=2.10 min, m/z 225.12 (M+H)⁺. 1H NMR (400 MHz, CDCl₃) δ ppm 7.55 (d, J=8.53 Hz, 1H), 7.44 (d, J=2.00 Hz, 1H), 7.31 (dd, J=8.53, 2.22 Hz, 1H), 7.02 (dd, J=5.28, 1.91 Hz, 1H), 6.73 (ddd, J=4.96, 3.23, 1.43 Hz, 1H), 4.85 (dd, J=3.78, 1.88 Hz, 1H), 3.56-3.50 (m, 1H), 2.53 (s, 3H), 2.47 (td, J=8.24, 1.63 Hz, 1H), 2.33 (td, J=8.24, 1.54 Hz, 1H).

### Step 2

### Preparation of 2-Methyl-4-((1S,4R)-3-oxo-2-azabicyclo[2.2.1]heptan-2-yl)benzonitrile

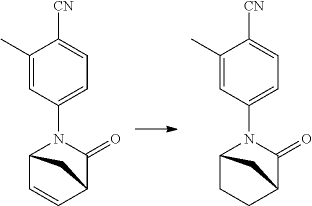

2-Methyl-4-((1S,4R)-3-oxo-2-azabicyclo[2.2.1]heptan-2-yl)benzonitrile was prepared using General Procedure N. LC/MS (Table 1, Method h) Rₜ=2.10 min, m/z 227.13 (M+H)⁺.

### Step 3

### Preparation of (Z)—N′-hydroxy-2-methyl-4-((1S,4R)-3-oxo-2-azabicyclo[2.2.1]heptan-2-yl)benzimidamide

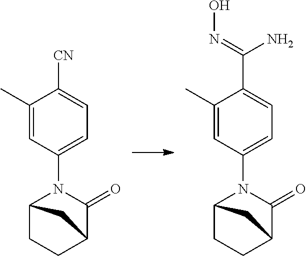

(Z)—N′-hydroxy-2-methyl-4-((1S,4R)-3-oxo-2-azabicyclo[2.2.1]heptan-2-yl)benzimidamide was prepared using General Procedure C. LC/MS (Table 1, Method g) Rₜ=1.33 min, m/z 260.17 (M+H)⁺.

### Step 4

### (1S,4R)-2-(4-(5-(3-chloro-4-isopropoxyphenyl)-1,2,4-oxadiazol-3-yl)-3-methylphenyl)-2-azabicyclo[2.2.1]heptan-3-one

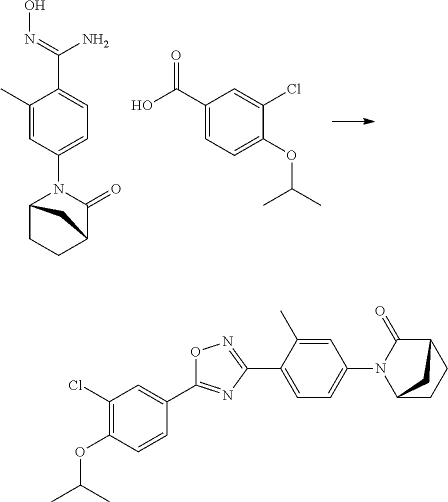

(1S,4R)-2-(4-(5-(3-chloro-4-isopropoxyphenyl)-1,2,4-oxadiazol-3-yl)-3-methylphenyl)-2-azabicyclo[2.2.1]heptan-3-one was prepared using General Procedure D. LC/MS (Table 1, Method g) Rₜ=3.45 min, m/z 438.14 (M+H)⁺. 1H NMR (400 MHz, DMSO-d6) δ ppm 8.17 (d, J=2.17 Hz, 1H), 8.10 (dd, J=8.69, 2.17 Hz, 1H), 8.02 (d, J=8.39 Hz, 1H), 7.66-7.60 (m, 2H), 7.44 (d, J=8.96 Hz, 1H), 4.88 (td, J=6.0, 1H), 4.73 (brs, 1H), 2.88-2.83 (m, 1H), 2.61 (s, 3H), 2.02-1.90 (m, 3H), 1.79-1.68 (m, 1H), 1.61-1.54 (m, 2H), 1.36 (d, J=6.01 Hz, 6H), 1.29-1.20 (m, 1H), 0.89-0.83 (m, 1H).

### Step 5

### (1R,3S)-3-(4-(5-(3-chloro-4-isopropoxyphenyl)-1,2,4-oxadiazol-3-yl)-3-methylphenylamino)cyclopentanecarboxylic acid

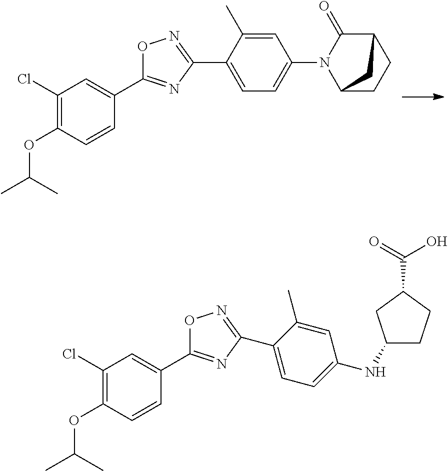

(1R,3S)-3-(4-(5-(3-chloro-4-isopropoxyphenyl)-1,2,4-oxadiazol-3-yl)-3-methylphenylamino)cyclopentanecarboxylic acid was prepared using General Procedure P.

LC/MS (Table 1, Method g) Rₜ=3.08 min, m/z 456.44 (M+H)⁺. 1HNMR (400 MHz, DMSO-d6) δ ppm 12.13 (brs, 1H), 8.14 (d, J=2.16 Hz, 1H), 8.07 (dd, J=8.70, 2.17 Hz, 1H), 7.82 (d, J=8.52 Hz, 1H), 7.43 (d, J=9.03 Hz, 1H), 6.55 (d, J=7.93 Hz, 2H), 6.28 (d, J=6.84 Hz, 1H), 4.87 (td, J=6.0 Hz, 1H), 3.81 (sextet, J₁=6.8 Hz, J₂=7.2 Hz, 1H), 2.77 (quintet, J=8.2 Hz, 1H), 2.51 (s, 7H), 2.38-2.28 (m, 1H), 2.05-1.94 (m, 1H), 1.92-1.83 (m, 2H), 1.69-1.58 (m, 1H), 1.58-1.45 (m, 1H), 1.36 (d, J=6.02 Hz, 6H).

### Example #80

### Preparation of (1S,4R)-2-(4-(5-(5-chloro-6-isopropoxypyridin-3-yl)-1,2,4-oxadiazol-3-yl)-3-methylphenyl)-2-azabicyclo[2.2.1]heptan-3-one

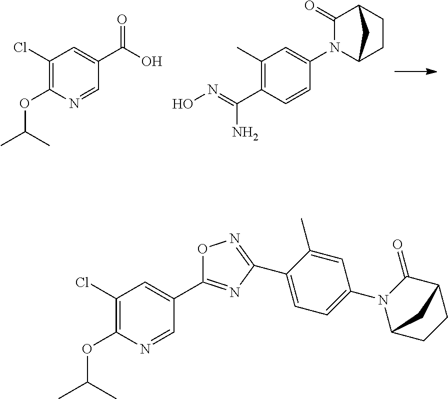

(1S,4R)-2-(4-(5-(5-chloro-6-isopropoxypyridin-3-yl)-1,2,4-oxadiazol-3-yl)-3-methylphenyl)-2-azabicyclo[2.2.1]heptan-3-one is prepared using General Procedure D. LC/MS (Table 1, Method g) Rₜ=3.61 min, m/z 439.18 (M+H)⁺. 1H NMR (400 MHz, CDCl₃) δ ppm 8.86 (d, J=2.12 Hz, 1H), 8.38 (d, J=2.12 Hz, 1H), 8.09 (d, J=8.60 Hz, 1H), 7.56 (d, J=2.18 Hz, 1H), 7.47 (dd, J=8.58, 2.26 Hz, 1H), 5.49 (td, J=6.0, 1H), 4.57-4.52 (m, 1H), 3.02-2.97 (m, 1H), 2.69 (s, 3H), 2.13-1.86 (m, 4H), 1.84-1.73 (m, 1H), 1.60-1.54 (m, 1H), 1.45 (d, J=6.21 Hz, 6H).

### Example #81

### Preparation of (1R,3S)-3-(4-(5-(5-chloro-6-isopropoxypyridin-3-yl)-1,2,4-oxadiazol-3-yl)-3-methylphenylamino)cyclopentanecarboxylic acid

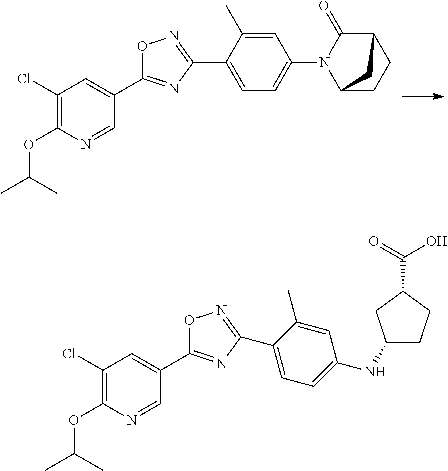

(1R,3S)-3-(4-(5-(5-chloro-6-isopropoxypyridin-3-yl)-1,2,4-oxadiazol-3-yl)-3-methylphenylamino)cyclopentanecarboxylic acid is prepared using General Procedure P.

LC/MS (Table 1, Method g) Rₜ=3.23 min, m/z 457.15 (M+H)⁺. ¹HNMR (400 MHz, DMSO-d6) δ ppm 12.08 (brs, 1H), 8.88 (d, J=2.10 Hz, 1H), 8.50 (d, J=2.09 Hz, 1H), 7.83 (d, J=8.66 Hz, 1H), 6.61-6.51 (m, 2H), 6.28 (d, J=6.32 Hz, 1H), 5.44 (td, J=6.0 Hz, 1H), 3.88-3.74 (m, 1H), 2.83-2.70 (m, 1H), 2.52 (s, 3H), 2.38-2.27 (m, 1H), 2.05-1.94 (m, 1H), 1.87 (dd, J=15.25, 7.64 Hz, 2H), 1.68-1.58 (m, 1H), 1.58-1.46 (m, 1H), 1.39 (d, J=6.19 Hz, 6H).

### Example #82

### Preparation of 5-(3-(2H-tetrazol-5-yl)phenyl)-3-(3-chloro-4-isopropoxyphenyl)-1,2,4-oxadiazole

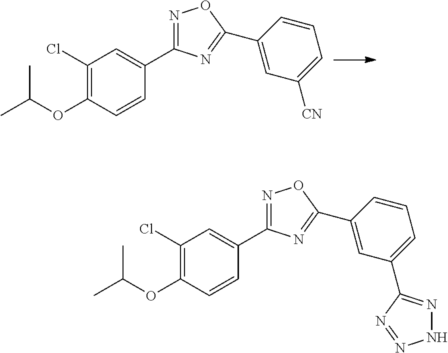

A mixture of 3-(3-(3-chloro-4-isopropoxyphenyl)-1,2,4-oxadiazol-5-yl)benzonitrile Table A, entry A.4), sodium azide (0.036 g, 0.554 mmol), zinc bromide (0.074 g, 0.329 mmol), water (2.83 mL) and THF (0.944 mL) was heated with stirring in a CEM microwave at about 120° C. for about 105 min. The resulting suspension was acidified to a pH of about 1 using 1 N aqueous HCl. Acetic acid and ethanol were added, after which the mixture was stirred overnight. The solution was then diluted with water and stirred to give a white precipitate and the resulting solid was collected by vacuum filtration and washed with water to provide a white solid. The crude solid was further purified by automated flash chromatography (0-10% MeOH in DCM) to give the product, 5-(3-(2H-tetrazol-5-yl)phenyl)-3-(3-chloro-4-isopropoxyphenyl)-1,2,4-oxadiazole (0.090 g; 78%). LC/MS (Table 1, Method g) Rₜ=2.14 min, m/z 383 (M+H)⁺; ¹H NMR (400 MHz, DMSO-d6) δ 8.84 (s, 1H), 8.42-8.35 (m, 2H), 8.10 (d, J=2.1, 1H), 8.04 (dd, J=2.1, 8.6, 1H), 7.90 (t, J=7.9, 1H), 7.41 (d, J=8.8, 1H), 4.91-4.76 (m, 1H), 1.36 (d, J=6.0, 6H).

### Example #83

### Preparation of methyl 2-(5-(3-(3-(3-chloro-4-isopropoxyphenyl)-1,2,4-oxadiazol-5-yl)phenyl)-2H-tetrazol-2-yl)acetate

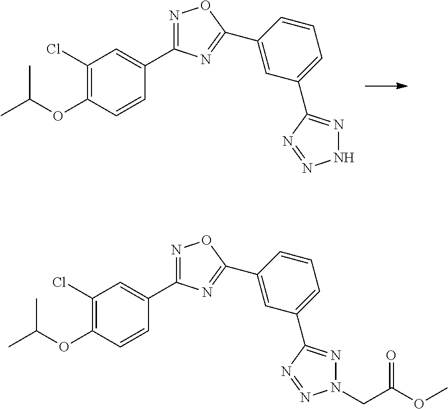

A mixture of 5-(3-(2H-tetrazol-5-yl)phenyl)-3-(3-chloro-4-isopropoxyphenyl)-1,2,4-oxadiazole (0.114 g, 0.299 mmol), methyl bromoacetate (0.06 mL, 0.651 mmol) and potassium carbonate (0.061 g, 0.441 mmol) in DMF (2.3 mL) was stirred overnight at RT. The mixture was then concentrated to dryness and the residue was triturated with water (10 mL). The resulting suspension was filtered through a sintered glass funnel and the resulting solid washed with water (3×15 mL), air dried, and resuspended in dichloromethane/MeOH (9:1; 5 mL). The suspension was filtered through a sintered glass funnel and the filtrate then concentrated under reduced pressure. The residue was purified by automated flash chromatography (0-25% EtOAc/heptane) to give the product as a white solid: methyl 2-(5-(3-(3-(3-chloro-4-isopropoxyphenyl)-1,2,4-oxadiazol-5-yl)phenyl)-2H-tetrazol-2-yl)acetate (0.082 g, 60.3%). LC/MS (Table 1, Method g) Rₜ=3.10 min, m/z 455 (M+H)⁺; ¹H NMR (400 MHz, DMSO-d6) δ 8.82 (s, 1H), 8.40 (dd, J=7.8, 19.8, 2H), 8.10 (d, J=2.1, 1H), 8.05 (dd, J=2.1, 8.6, 1H), 7.90 (t, J=7.9, 1H), 7.40 (d, J=8.8, 1H), 5.99 (s, 2H), 4.84 (dt, J=6.2, 12.2, 1H), 3.78 (s, 3H), 1.36 (d, J=6.0, 6H).

### Preparation #85

### Preparation of 3-(3-(3-chloro-4-isopropoxyphenyl)-1,2,4-oxadiazol-5-yl)-N-hydroxybenzimidamide

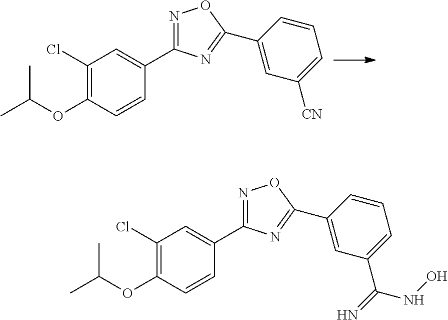

A mixture of 3-(3-(3-chloro-4-isopropoxyphenyl)-1,2,4-oxadiazol-5-yl)benzonitrile (0.101 g, 0.298 mmol), hydroxylamine hydrochloride (0.031 g, 0.445 mmol) and triethylamine (0.095 mL, 0.684 mmol) in ethanol (2.98 mL) was heated to about reflux for about 20 h in a 10 mL round bottom flask. The reaction mixture was concentrated under reduced pressure to provide a crude solid, which was then triturated with water (5 mL) and then isolated by vacuum filtration, rinsed with water and dried in open air to afford 3-(3-(3-chloro-4-isopropoxyphenyl)-1,2,4-oxadiazol-5-yl)-N-hydroxybenzimidamide (0.098 g, 88%), which was used without further purification. LC/MS (Table 1, Method j) Rₜ=1.45 min, m/z 373 (M+H)⁺;

### Example #84

### Preparation of 3-(3-(3-(3-chloro-4-isopropoxyphenyl)-1,2,4-oxadiazol-5-yl)phenyl)-1,2,4-oxadiazol-5(2H)-one

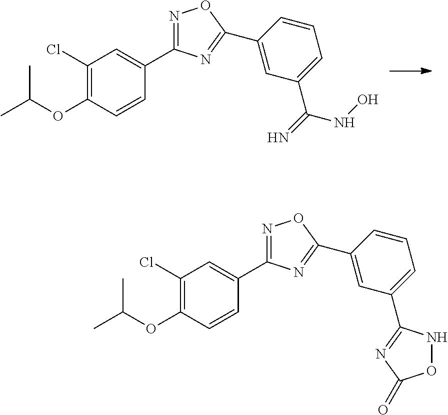

To a mixture of 3-(3-(3-chloro-4-isopropoxyphenyl)-1,2,4-oxadiazol-5-yl)-N-hydroxybenzimidamide (0.098 g, 0.263 mmol) and 1,1′-carbonyldiimidazole (0.058 g, 0.345 mmol) in 1,4-dioxane (0.263 mL) was added DBU (0.042 mL, 0.281 mmol). The resulting solution was heated at about 105° C. for about 4 h and monitored by LC/MS (Table 1, method j). Additional 1,1′-carbonyldiimidazole (0.021 g) and DBU (0.025 mL) were added and heating continued for an additional 0.5 h, after which LC/MS (Table 1, method j) indicated complete conversion to the product. The reaction mixture was diluted with water (6 mL), extracted with ethyl acetate (5 mL), acidified to a pH of about 2 with aqueous 4 N HCl, and again extracted with ethyl acetate (2×5 mL). The two organic layers were combined, concentrated under reduced pressure, and the resulting semisolid redissolved in dichloromethane and filtered to remove a white solid. The filtrate was concentrated and then purified by preparative HPLC (Varian ProStar with UV-Visible detector) using a Thermo Electron Hyperprep HS C18 column and the following gradient: A: Water; B: Acetonitrile; 30% B to 70% B over 45 min to give the product: 3-(3-(3-(3-chloro-4-isopropoxyphenyl)-1,2,4-oxadiazol-5-yl)phenyl)-1,2,4-oxadiazol-5(2H)-one (0.030 g, 28.6% yield). LC/MS (Table 1, Method g) Rₜ=2.61 min, m/z 397 (M−H)⁻; ¹H NMR (400 MHz, DMSO-d6) δ 13.42-13.04 (m, 1H), 8.62 (s, 1H), 8.40 (d, J=8.0, 1H), 8.15 (d, J=8.0, 1H), 8.09 (d, J=1.9, 1H), 8.03 (dd, J=2.0, 8.6, 1H), 7.87 (t, J=7.9, 1H), 7.41 (d, J=8.6, 1H), 4.92-4.74 (m, 1H), 1.36 (d, J=6.0, 6H).

### Preparation #76

### Preparation of 3-(4-fluorophenyl)-5-(4-isobutylphenyl)-1,2,4-oxadiazole

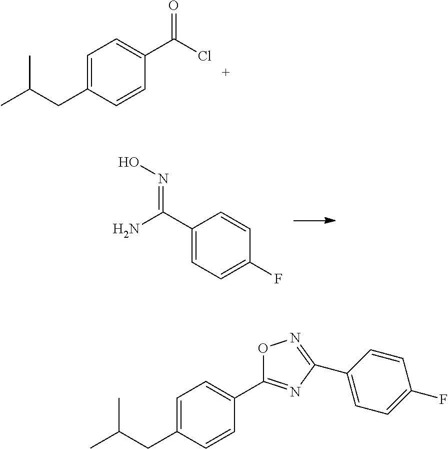

3-(4-Fluorophenyl)-5-(4-isobutylphenyl)-1,2,4-oxadiazole was prepared from 4-isobutylbenzoyl chloride (prepared from 4-isobutylbenzoic acid [TCI] according to General Procedure F) and 4-fluorobenzamidoxime according to General Procedure E. LC/MS (Table 1, Method h) Rₜ=3.32 min, m/z 297 (M+H)⁺; ¹H NMR (400 MHz, DMSO-d6) δ 8.25-8.06 (m, 4H), 7.52-7.39 (m, 4H), 2.59 (d, J=7.2, 2H), 1.92 (dt, J=6.8, 13.6, 1H), 0.89 (t, J=8.5, 6H).

### Example #85

### Preparation of (1R,3S)-3-(4-(5-(4-isobutylphenyl)-1,2,4-oxadiazol-3-yl)phenylamino)cyclopentanecarboxylic acid

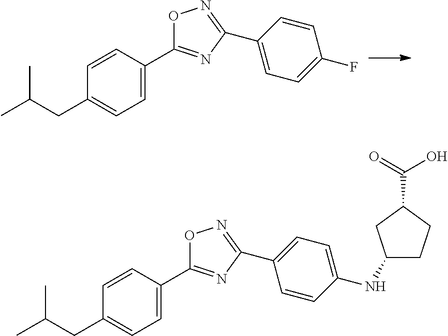

(1R,3S)-3-(4-(5-(4-isobutylphenyl)-1,2,4-oxadiazol-3-yl)phenylamino)cyclopentanecarboxylic acid was prepared from 3-(4-fluorophenyl)-5-(4-isobutylphenyl)-1,2,4-oxadiazole and (1R,3S)-3-aminocyclopentanecarboxylic acid (Acros) according to General Procedure L.

LC/MS (Table 1, Method g) Rₜ=3.09 min, m/z 406 (M+H)⁺; ¹H NMR (400 MHz, CDCl₃) δ 8.13-8.07 (m, 2H), 8.00-7.93 (m, 2H), 7.31 (s, 1H), 7.29 (s, 1H), 6.66 (d, J=8.5, 2H), 3.99 (p, J=5.5, 1H), 3.03-2.91 (m, 1H), 2.56 (d, J=7.2, 2H), 2.38 (ddd, J=6.6, 9.1, 13.6, 1H), 2.12-1.87 (m, 5H), 1.80 (dd, J=5.9, 11.6, 1H), 0.93 (d, J=6.6, 6H).

### Preparation #77

### Preparation of 3-(3-chloro-4-isopropoxyphenyl)-5-(2-ethyl-4-fluorophenyl)-1,2,4-oxadiazole

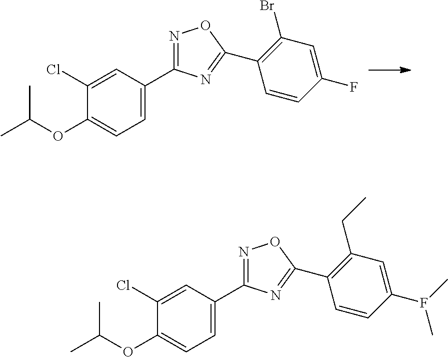

A 5 mL microwave reaction vial equipped with pressure-releasing septa cap was charged with 5-(2-bromo-4-fluorophenyl)-3-(3-chloro-4-isopropoxyphenyl)-1,2,4-oxadiazole (0.500 g, 1.215 mmol, prepared from 3-chloro-N-hydroxy-4-isopropoxy-benzamidine and 2-bromo-4-fluorobenzoyl chloride according to General Procedure E), ethylboronic acid (0.179 g, 2.429 mmol, Alfa Aesar), tetrakis(triphenylphosphine)palladium(0) (0.140 g, 0.121 mmol), and potassium phosphate tribasic (0.516 g, 2.429 mmol) in 1,2-dimethoxyethane (13.35 mL) to give a orange suspension. The mixture was heated in a Biotage microwave at about 150° C. for about 45 min. The reaction was concentrated to dryness to give an orange residue, which was purified by automated flash chromatography (0-10% EtOAc/heptane) to afford 3-(3-chloro-4-isopropoxyphenyl)-5-(2-ethyl-4-fluorophenyl)-1,2,4-oxadiazole (0.224 g, 51.1% yield). LC/MS (Table 1, Method h) Rₜ=3.33 min, m/z 361 (M+H)⁺; ¹H NMR (400 MHz, CDCl₃) δ 8.18 (d, J=2.1, 1H), 8.13 (dd, J=5.8, 8.7, 1H), 8.01 (dd, J=2.1, 8.5, 1H), 7.15-7.02 (m, 3H), 4.73-4.63 (m, 1H), 3.23-3.14 (m, 2H), 1.44 (d, J=6.0, 6H), 1.32 (t, J=7.5, 3H).

### Example #86

### Preparation of (1R,3S)-3-(4-(3-(3-chloro-4-isopropoxyphenyl)-1,2,4-oxadiazol-5-yl)-3-ethylphenylamino)cyclopentanecarboxylic acid

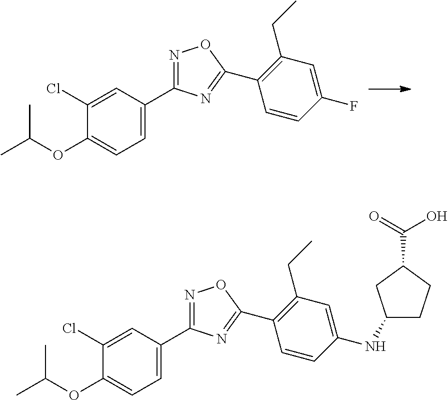

(1R,3S)-3-(4-(3-(3-chloro-4-isopropoxyphenyl)-1,2,4-oxadiazol-5-yl)-3-ethylphenylamino)cyclopentanecarboxylic acid was prepared from 3-(3-chloro-4-isopropoxyphenyl)-5-(2-ethyl-4-fluorophenyl)-1,2,4-oxadiazole and (1R,3S)-3-aminocyclopentanecarboxylic acid (Acros) according to General Procedure L. LC/MS (Table 1, Method g) Rₜ=3.14 min, m/z 470 (M+H)⁺; ¹H NMR (400 MHz, DMSO-d6) δ 12.32-11.86 (m, 1H), 8.01 (d, J=2.1, 1H), 7.96 (dd, J=2.1, 8.6, 1H), 7.89-7.82 (m, 1H), 7.38 (d, J=8.7, 1H), 6.61-6.55 (m, 2H), 4.81 (p, J=6.0, 1H), 3.91-3.80 (m, 1H), 3.04 (q, J=7.4, 2H), 2.75 (s, 1H), 2.33-2.23 (m, 1H), 2.02-1.92 (m, 1H), 1.92-1.82 (m, 2H), 1.72-1.60 (m, 1H), 1.60-1.48 (m, 1H), 1.35 (d, J=6.0, 6H), 1.21 (d, J=7.4, 4H).

### Preparation #78

### Preparation of 2-(3-(3-chloro-4-isopropoxyphenyl)-1,2,4-oxadiazol-5-yl)-5-fluorobenzonitrile

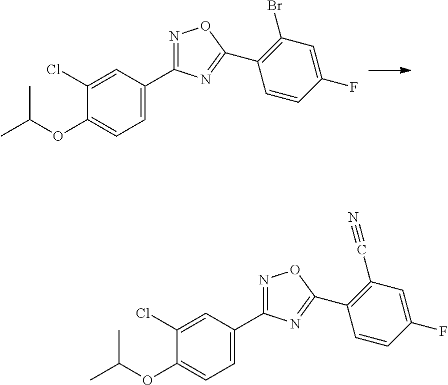

A suspension of 5-(2-bromo-4-fluorophenyl)-3-(3-chloro-4-isopropoxyphenyl)-1,2,4-oxadiazole (0.500 g, 1.215 mmol) and copper(I) cyanide (0.163 g, 1.822 mmol) in N-methyl-2-pyrrolidinone (5.65 mL) was heated in a Biotage microwave at about 150° C. for about 15 min. The reaction was diluted with ethyl acetate (90 mL) and water (90 mL) was added. After separating the layers, the aqueous phase was extracted with ethyl acetate (2×25 mL) and the combined organic phases were washed with brine, dried over MgSO₄, filtered, and concentrated under reduced pressure. The crude material was purified automated flash chromatography (0-15% EtOAc/Heptane) to give the product, 2-(3-(3-chloro-4-isopropoxyphenyl)-1,2,4-oxadiazol-5-yl)-5-fluorobenzonitrile (0.220 g, 50.6% yield). LC/MS (Table 1, Method g) Rₜ=3.06 min, m/z 358 (M+H)⁺; ¹H NMR (400 MHz, CDCl₃) δ 8.35 (dd, J=5.2, 8.8, 1H), 8.20 (d, J=2.1, 1H), 8.05 (dd, J=2.1, 8.6, 1H), 7.66-7.48 (m, 2H), 7.05 (d, J=8.6, 1H), 4.68 (dt, J=6.0, 12.1, 1H), 1.45-1.38 (m, 6H).

### Example #87

### Preparation of (1R,3S)-3-(4-(3-(3-chloro-4-isopropoxyphenyl)-1,2,4-oxadiazol-5-yl)-3-cyanophenylamino)cyclopentanecarboxylic acid

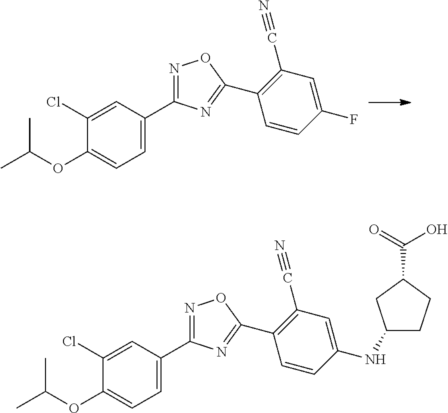

(1R,3S)-3-(4-(3-(3-chloro-4-isopropoxyphenyl)-1,2,4-oxadiazol-5-yl)-3-cyanophenylamino)cyclopentanecarboxylic acid was prepared from 2-(3-(3-chloro-4-isopropoxyphenyl)-1,2,4-oxadiazol-5-yl)-5-fluorobenzonitrile and (1R,3S)-3-aminocyclopentanecarboxylic acid (Acros) according to General Procedure L. LC/MS (Table 1, Method g) Rₜ=2.85 min, m/z 467 (M+H)⁺; ¹H NMR (400 MHz, DMSO-d6) δ 12.31-12.02 (m, 1H), 8.04 (d, J=6.8, 2H), 7.98 (dd, J=2.1, 8.6, 1H), 7.43-7.38 (m, 1H), 7.38-7.30 (m, 1H), 7.15 (d, J=2.4, 1H), 7.00 (dd, J=2.4, 9.0, 1H), 4.82 (p, J=6.0, 1H), 3.92 (q, J=6.8, 1H), 2.82-2.72 (m, 1H), 2.38-2.28 (m, 1H), 2.03 (s, 1H), 1.94-1.84 (m, 2H), 1.68 (s, 1H), 1.55 (d, J=7.1, 1H), 1.35 (d, J=6.0, 6H).

### Preparation #79

### Preparation of (E)-ethyl 4-(1,3-dioxoisoindolin-2-yl)-2-methylbut-2-enoate

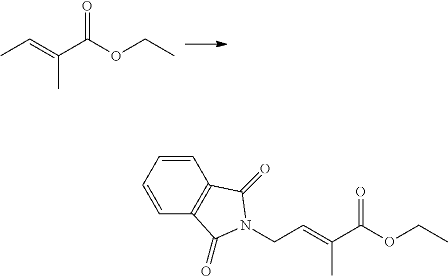

A 200 mL round bottom flask equipped with reflux condenser outfitted with a nitrogen inlet adapter was charged with ethyl tiglate (16.13 mL, 117 mmol), N-bromosuccinimide (10.72 mL, 126 mmol), and benzoyl peroxide (0.012 g, 0.051 mmol) in carbon tetrachloride (58.5 mL) to give a yellow suspension. The reaction mixture was heated at reflux for about 1 day.

The reaction mixture was concentrated under reduced pressure via rotary evaporation to give a reddish-brown oil. The reaction flask was then outfitted with a reflux condenser and nitrogen inlet adapter, and then was charged with potassium phthalimide (19.82 g, 107 mmol) and DMF (86 mL) to give a purple solution. The mixture was heated in an oil bath at about 140° C. for about 18 h. The bath was removed and the reaction cooled to ambient temperature, filtered through a sintered glass funnel, and concentrated under reduced pressure to about half its original volume. The resulting dark brown solution was diluted with EtOAc (450 mL) and extracted with water (2×400 mL), 50% saturated aqueous NaCl (2×400 mL) and brine (400 mL). The organic phase was dried over MgSO₄, filtered and concentrated to give a brown oil. The crude material was purified by automated flash chromatography (10-50% EtOAc/heptane) to give a yellow semi-solid, from which the product was isolated by crystallization from cyclohexane: (E)-ethyl 4-(1,3-dioxoisoindolin-2-yl)-2-methylbut-2-enoate (4.0 g, 13.68% yield). ¹H NMR (400 MHz, CDCl₃) δ 7.90-7.83 (m, 2H), 7.77-7.70 (m, 2H), 6.69-6.61 (m, 1H), 4.44 (d, J=6.8, 2H), 4.22-4.13 (m, 2H), 1.27 (td, J=0.8, 7.1, 3H).

### Preparation #80

### Preparation of ethyl 4-(1,3-dioxoisoindolin-2-yl)-2-methylbutanoate

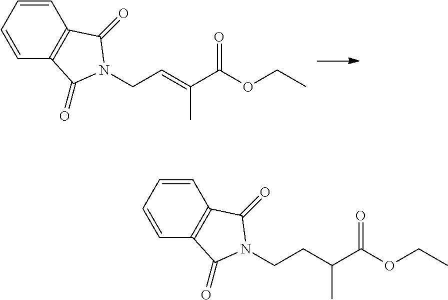

A solution of (E)-ethyl 4-(1,3-dioxoisoindolin-2-yl)-2-methylbut-2-enoate (4.0 g, 14.64 mmol) in methanol (150 mL) was passed through an H-Cube hydrogenation apparatus containing a 10% palladium on carbon cartridge at 1.5 mL/min under an atmosphere of hydrogen (about 20 bar) at about 20° C. for about 90 min. The catalyst cartridge was replaced with a fresh one and the process repeated, after which the resulting solution was concentrated via rotary evaporation to afford ethyl 4-(1,3-dioxoisoindolin-2-yl)-2-methylbutanoate (4.03 g, 100% yield), as a colorless oil. The material was used directly in the next step without further purification. LC/MS (Table 1, Method h) Rₜ=2.00 min, m/z 276 (M+H)⁺; ¹H NMR (400 MHz, CDCl₃) δ 7.92-7.79 (m, 2H), 7.77-7.64 (m, 2H), 4.12 (q, J=7.2, 2H), 3.75 (t, J=7.1, 2H), 2.47 (h, J=7.1, 1H), 2.16-2.02 (m, 1H), 1.79 (dq, J=6.9, 13.7, 1H), 1.34-1.13 (m, 6H).

### Preparation #81

### Preparation of 4-methyl-2-aminobutanoic acid

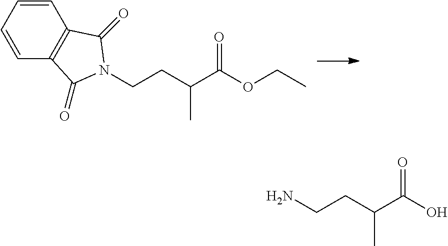

A 500 mL round bottom flask equipped with reflux condenser outfitted with a nitrogen inlet adapter was charged with ethyl 4-(1,3-dioxoisoindolin-2-yl)-2-methylbutanoate (4.03 g, 14.64 mmol) in acetic acid (113 mL) and 6 N HCl (227 mL) to give a colorless solution. The solution was heated at about 135° C. for about 24 h. After cooling to RT, a white precipitate formed and was removed by vacuum filtration. The filtrate was concentrated under reduced pressure, filtered again to remove more solid, and further concentrated to give a faintly yellow residue. The residue was purified by ion exchange chromatography (Dowex 8×50 w H⁺ form; rinsed w/deionized water; prepared and loaded with 1 N aqueous HCl; eluted sequentially with deionized water followed by 1 M aqueous pyridine solution). Collection and evaporation of the appropriate fractions (identified using ninhydrin stain) gave the product, 4-amino-2-methylbutanoic acid (1.526 g, 89% yield), as a faintly yellow solid. The material was used directly in the next step without further purification. ¹H NMR (400 MHz, D₂O) δ 3.10-2.95 (m, 2H), 2.47-2.35 (m, 1H), 1.98-1.84 (m, 1H), 1.82-1.69 (m, 1H), 1.16 (dd, J=1.1, 7.0, 3H).

### Preparation #82

### Preparation of 4-phenyl-5-(trifluoromethyl)thiophene-2-carboxamide

### Step 1

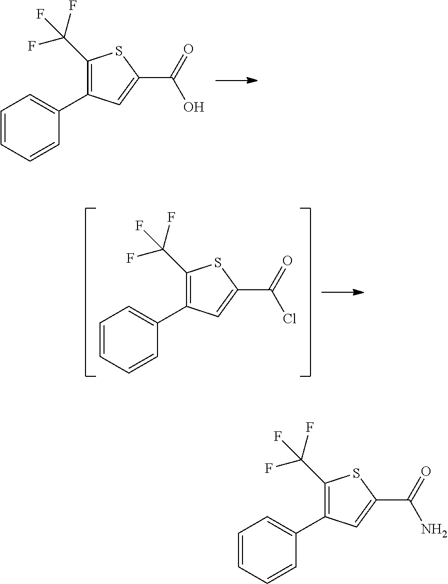

4-Phenyl-5-(trifluoromethyl)thiophene-2-carboxylic acid (5 g, 18.37 mmol) (Maybridge) and DMF (0.071 mL, 0.918 mmol) were combined in dichloromethane (184 mL) under nitrogen to give a colorless solution. Oxalyl chloride (1.768 mL, 20.20 mmol) was added slowly and then the reaction stirred for about 5 h. The solvents were removed under reduced pressure. Toluene was added and the solvents removed. The residue was taken up in ethyl acetate (10 mL) and added dropwise to a rapidly stirred mixture of ethyl acetate (150 mL) and concentrated ammonium hydroxide (100 mL). The mixture was stirred for 1 h. The layers were separated and the aqueous layer extracted with ethyl acetate (50 mL). The combined extracts were washed with brine, dried over sodium sulfate, filtered, and evaporated to a white solid: LC/MS (Method g) Rₜ=2.24 min.; MS m/z: 270.06 (M−H)⁻, ¹H NMR (400 MHz, DMSO-d6) δ ppm 8.24 (s, 1H), 7.90-7.87 (m, 1H), 7.80 (s, 1H), 7.52-7.42 (m, 5H)

### Step 2

### Preparation of 4-phenyl-5-(trifluoromethyl)thiophene-2-carbonitrile

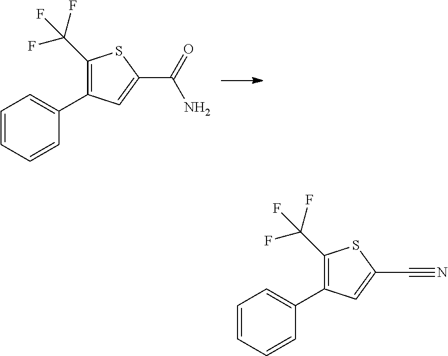

4-phenyl-5-(trifluoromethyl)thiophene-2-carboxamide (1.400 g, 5.16 mmol) was dissolved in 1,2-dichloroethane (51 mL) under nitrogen to give a colorless solution. Burgess reagent (4.92 g, 20.64 mmol) (Acros) was added and the reaction heated at about 60° C. for about 4 h. The reaction was allowed to cool to ambient temperature. Methylene chloride (50 mL) and water (50 mL) were added and the layers separated. The aqueous layer was extracted with methylene chloride (1×25 mL). The combined extracts were washed with brine, dried over sodium sulfate, filtered, and evaporated to a colorless solid. The residue was purified by flash column chromatography (40 g Redi-Sep) eluting with 20-50% ethyl acetate/heptane and the product fractions combined. The solvents were removed under reduced pressure to provide 4-phenyl-5-(trifluoromethyl)thiophene-2-carbonitrile (1.227 g, 4.85 mmol, 94% yield) as a white solid: LC/MS (Method H) Rₜ=2.74 min.; 1H NMR (400 MHz, DMSO-d6) δ ppm 8.27-8.17 (m, 1H), 7.55-7.40 (m, 6H).

### Step 3

### Preparation of (Z)—N′-hydroxy-4-phenyl-5-(trifluoromethyl)thiophene-2-carboximidamide

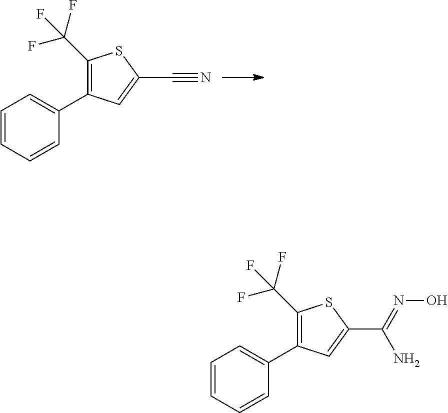

4-Phenyl-5-(trifluoromethyl)thiophene-2-carbonitrile (1.22 g, 4.82 mmol) was dissolved in ethanol (48 mL) under nitrogen to give a colorless suspension. Hydroxylamine (0.947 mL, 19.27 mmol) was added and the reaction heated at about 60° C. for about 5 h. TLC in 1:1 EtOAc/heptane showed (uv light visualization) that the reaction was complete. The reaction was allowed to cool to ambient temperature. The solvents were removed under reduced pressure. The residue was purified by flash column chromatography (40 g Redi-Sep column) eluting with 20-50% ethyl acetate/heptane and the product fractions combined. The solvents were removed under reduced pressure to provide (Z)—N′-hydroxy-4-phenyl-5-(trifluoromethyl)thiophene-2-carboximidamide (1.397 g, 4.88 mmol, 101% yield) as a white solid: LC/MS (Method g) Rₜ=2.19 min.; MS m/z: 287.08 (M+H)⁺; 1H NMR (400 MHz, DMSO) δ ppm 9.98 (s, 1H), 7.65-7.63 (m, 1H), 7.51-7.41 (m, 5H), 6.15 (s, 2H).

### Preparation #83

### Preparation of (1aS,5aR)-1,1,2-Trimethyl-1,1a,5,5a-tetrahydro-3-thia-cyclopropa[a]pentalene-4-carboxylic acid amide

### Step 1

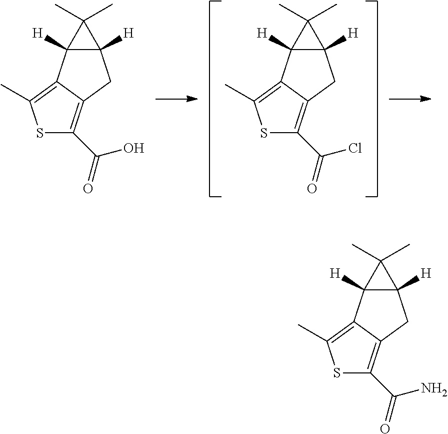

(1aS,5aR)-1,1,2-Trimethyl-1,1a,5,5a-tetrahydro-3-thia-cyclopropa[a]pentalene-4-carboxylic acid (0.39 g, 1.754 mmol) (Prepared according to WO2006/010379) and DMF (6.79 μl, 0.088 mmol) were combined in dichloromethane (17 mL) under nitrogen to give an orange solution. Oxalyl chloride (0.169 mL, 1.930 mmol) was added and the reaction stirred for about 4 h. The solvents were removed under reduced pressure and the residue dried under vacuum for about 30 minutes. The residue was dissolved in ethyl acetate (15 mL). Concentrated ammonium hydroxide (10 mL) was added with rapid stirring and the mixture was stirred for about 45 min. The layers were separated and the aqueous layer extracted with ethyl acetate (15 mL). The combined organic extracts were washed with brine, dried over sodium sulfate, filtered, and evaporated to an orange oil. The residue was purified by flash column chromatography (40 g Redi-Sep column) eluting with 1:1 EtOAc/heptane and the product fractions combined. The solvents were removed under reduced pressure to provide (1aS,5aR)-1,1,2-Trimethyl-1,1a,5,5a-tetrahydro-3-thia-cyclopropa[a]pentalene-4-carboxylic acid amide (0.324 g, 1.464 mmol, 83% yield) as an orange oil: LC/MS (Method g) Rₜ=2.12 min.; MS m/z: 222.12 (M+H)⁺.

### Step 2

### Preparation of (1aS,5aR)-1,1,2-Trimethyl-1,1a,5,5a-tetrahydro-3-thia-cyclopropa[a]pentalene-4-carbonitrile

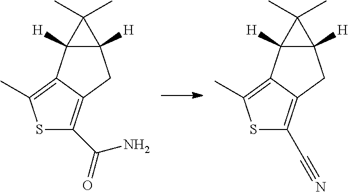

(1aS,5aR)-1,1,2-Trimethyl-1,1a,5,5a-tetrahydro-3-thia-cyclopropa[a]pentalene-4-carboxylic acid amide (0.324 g, 1.464 mmol) was dissolved in 1,2-dichloroethane (14 mL) under nitrogen to give an orange solution. Burgess reagent (0.698 g, 2.93 mmol) (Acros) was added and the reaction stirred for about 18 h. Methylene chloride (20 mL) and water (15 mL) were added and the layers separated. The aqueous layer was extracted with methylene chloride (1×10 mL). The combined extracts were washed with brine, dried over sodium sulfate, decanted, and evaporated to an orange oil. The oil was purified by flash column chromatography (40 g Redi-Sep) eluting with 0-10% ethyl acetate/heptane and the product fractions combined. The solvents were removed under reduced pressure to provide (1aS,5aR)-1,1,2-Trimethyl-1,1a,5,5a-tetrahydro-3-thia-cyclopropa[a]pentalene-4-carbonitrile (0.26 g, 1.279 mmol, 87% yield) as a colorless oil: LC/MS (Method h) Rₜ=2.84 min.; MS m/z: 204.09 (M+H)⁺.

### Step 3

### Preparation of (1aS,5aR)—N-Hydroxy-1,1,2-trimethyl-1,1a,5,5a-tetrahydro-3-thia-cyclopropa[a]pentalene-4-carboxamidine

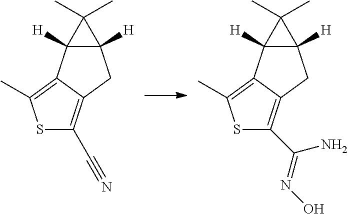

(1aS,5aR)-1,1,2-Trimethyl-1,1a,5,5a-tetrahydro-3-thia-cyclopropa[a]pentalene-4-carbonitrile (0.26 g, 1.279 mmol) and hydroxylamine (50% solution in water) (0.302 mL, 5.12 mmol) were combined in ethanol (12.8 mL) under nitrogen to give a colorless solution. The mixture was heated at about 60° C. for about 5 h. TLC in 1:1 EtOAc/heptane showed (uv light visualization) that the reaction was complete. The solvents were removed under reduced pressure. The residue was purified by flash column chromatography (40 g Redi-Sep column) eluting with 20-50% ethyl acetate/heptane and the product fractions combined. The solvents were removed under reduced pressure to provide (1aS,5aR)—N-Hydroxy-1,1,2-trimethyl-1,1a,5,5a-tetrahydro-3-thia-cyclopropa[a]pentalene-4-carboxamidine (0.284 g, 1.202 mmol, 94% yield) as a sticky white solid: LC/MS (Method g) Rₜ=2.16 min.; MS m/z: 237.09 (M+H)⁺.

### Preparation #84

### Preparation of (2R,4R)-1-tert-butyl 2-methyl 4-(4-(3-(3-chloro-4-isopropoxyphenyl)-1,2,4-oxadiazol-5-yl)phenylamino)pyrrolidine-1,2-dicarboxylate

### Step 1

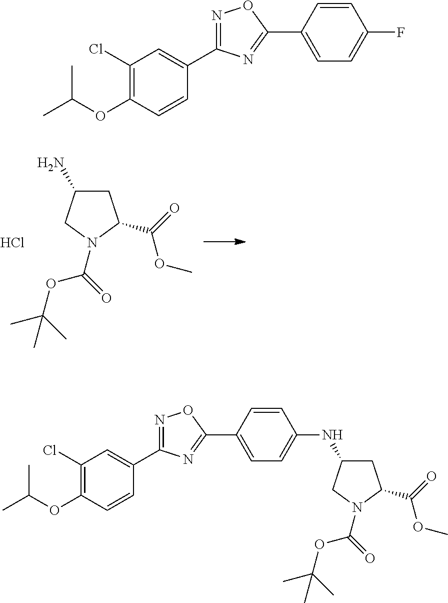

3-(3-Chloro-4-isopropoxyphenyl)-5-(4-fluorophenyl)-1,2,4-oxadiazole (0.4 g, 1.202 mmol), (2R,4R)-1-tert-butyl 2-methyl 4-aminopyrrolidine-1,2-dicarboxylate hydrochloride (0.506 g, 1.803 mmol) (Acesys Pharmatech), and potassium carbonate (0.498 g, 3.61 mmol) were combined in DMSO (4.01 mL) in a sealed vial to give a white suspension. The mixture was heated at about 100° C. for about 20 h. Ethyl acetate (20 mL) and water (20 mL) were added and the layers separated. The ethyl acetate layer was washed with water (2×10 mL). The ethyl acetate layer was washed with brine, dried over sodium sulfate, filtered, and evaporated to a yellow oil. The residue was purified by flash column chromatography (40 g Redi-Sep column) eluting with 10-50% ethyl acetate/heptane and the product fractions combined. The solvents were removed under reduced pressure to provide (2R,4R)-1-tert-butyl 2-methyl 4-(4-(3-(3-chloro-4-isopropoxyphenyl)-1,2,4-oxadiazol-5-yl)phenylamino)pyrrolidine-1,2-dicarboxylate (0.085 g, 0.153 mmol, 12.69% yield) as a off-white solid: LC/MS (Method h) Rₜ=3.10 min.; MS m/z: 557.24 (M+H)⁺, NMR (400 MHz,) δ 8.21 (d, J=2.0, 1H), 8.08-7.99 (m, 3H), 7.05 (d, J=8.7, 1H), 6.75-6.66 (m, 2H), 4.75-4.63 (m, 1H), 4.41 (dd, J=8.6, 35.3, 1H), 4.31-4.19 (m, 1H), 3.90-3.54 (m, 5H), 2.66-2.42 (m, 1H), 2.26-2.10 (m, 1H), 1.47 (t, J=12.3, 16H).

### Step 2

### Preparation of (2R,4R)-1-(tert-butoxycarbonyl)-4-(4-(3-(3-chloro-4-isopropoxyphenyl)-1,2,4-oxadiazol-5-yl)phenylamino)pyrrolidine-2-carboxylic acid

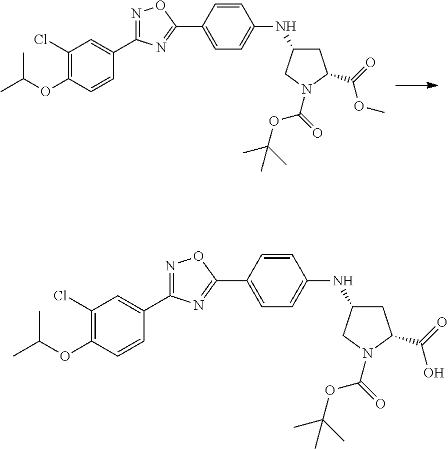

(2R,4R)-1-tert-butyl 2-methyl 4-(4-(3-(3-chloro-4-isopropoxyphenyl)-1,2,4-oxadiazol-5-yl)phenylamino)pyrrolidine-1,2-dicarboxylate (0.085 g, 0.153 mmol) was dissolved in a mixture of THF (2.0 mL) and water (1.0 mL) under nitrogen to give a colorless solution. Lithium hydroxide (15 mg, 0.626 mmol) was added and the reaction stirred for about 48 h. The solvents were removed under reduced pressure. Ethyl acetate (15 mL) and 1 N HCl (3 mL) were added and the layers separated. The aqueous layer was extracted with ethyl acetate (1×10 mL). The combined extracts were washed with brine, dried over sodium sulfate, filtered, and evaporated to a white solid. The residue was purified by flash column chromatography (40 g Redi-Sep column) eluting with 50-100% ethyl acetate/heptane and the product fractions combined. The solvents were removed under reduced pressure to provide (2R,4R)-1-(tert-butoxycarbonyl)-4-(4-(3-(3-chloro-4-isopropoxyphenyl)-1,2,4-oxadiazol-5-yl)phenylamino)pyrrolidine-2-carboxylic acid (0.036 g, 0.066 mmol, 43.4% yield) as an off-white solid: LC/MS (Method g) Rₜ=2.71 min.; MS m/z: 543.15 (M+H)⁺, NMR (400 MHz,) δ 8.21 (d, J=1.6, 1H), 8.08-7.98 (m, 3H), 7.05 (d, J=8.6, 1H), 6.63 (d, J=8.5, 2H), 4.76-4.64 (m, 1H), 4.59 (d, J=8.8, 1H), 4.28-4.17 (m, 1H), 3.68-3.46 (m, 2H), 2.73 (d, J=13.4, 1H), 2.50-2.36 (m, 1H), 1.53 (s, 9H), 1.50 (m, obscured, 1H), 1.45 (d, J=6.0, 6H).

### Example #88

### (2R,4R)-4-(4-(3-(3-chloro-4-isopropoxyphenyl)-1,2,4-oxadiazol-5-yl)phenylamino)pyrrolidine-2-carboxylic acid

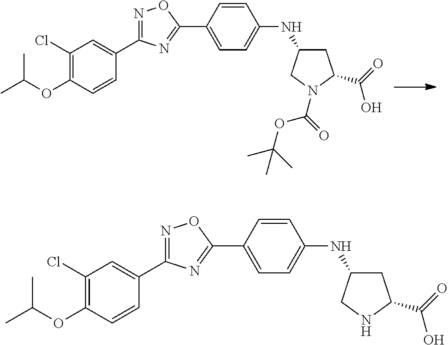

(2R,4R)-1-(tert-butoxycarbonyl)-4-(4-(3-(3-chloro-4-isopropoxyphenyl)-1,2,4-oxadiazol-5-yl)phenylamino)pyrrolidine-2-carboxylic acid (0.036 g, 0.066 mmol) was dissolved in dichloromethane (0.663 mL) under nitrogen to give a colorless solution. Trifluoroacetic acid (0.663 mL) was added and the reaction stirred for about 18 h. The solvents were removed under reduced pressure. The residue was purified by flash column chromatography (4 g Redi-sep) eluting with 1:1 EtOAc/(6:3:1 CHCl₃/MeOH/NH₄OH) and the product fractions combined. The solvents were removed under reduced pressure. Ether was added and the resulting solid was collected by vacuum filtration and washed with ether to provide (2R,4R)-4-(4-(3-(3-chloro-4-isopropoxyphenyl)-1,2,4-oxadiazol-5-yl)phenylamino)pyrrolidine-2-carboxylic acid (0.043 g, 0.097 mmol, quantitative yield) as a white solid on drying under vacuum at 55° C.: LC/MS (Method g) Rₜ=2.00 min.; MS m/z: 443.11 (M+H)⁺; 1H NMR (400 MHz, DMSO) δ ppm 8.00-7.95 (m, 1H), 7.95-7.89 (m, 1H), 7.86 (d, J=8.41 Hz, 2H), 7.33 (d, J=8.58 Hz, 1H), 6.86 (d, J=6.34 Hz, 1H), 6.72 (d, J=6.34 Hz, 2H), 4.84-4.72 (m, 1H), 4.19-4.08 (m, 1H), 3.81 (t, J=8.15 Hz, 1H), 3.47-3.39 (m, 1H), 3.13-3.06 (m, 1H), 2.62-2.51 (m, 1H), 1.96-1.85 (m, 1H), 1.33 (d, J=5.81 Hz, 6H).

### Preparation #85

### Preparation of methyl 5-chloro-6-hydroxynicotinate

### Step 1

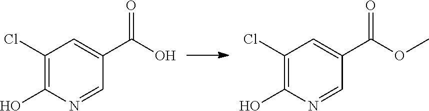

5-Chloro-6-hydroxynicotinic acid (10.2 g, 58.8 mmol) (Alfa Aesar) was dissolved in methanol (102 mL) under nitrogen to give a white suspension. Sulfuric acid (15.66 mL, 294 mmol) was added dropwise (exotherm observed) and the reaction heated at about 65° C. for about 6 h.

The reaction was cooled to about ambient temperature and stirred overnight. The resulting solid was collected by vacuum filtration and washed with methanol (2×10 mL) and ether (2×10 mL) to provide methyl 5-chloro-6-hydroxynicotinate (8.911 g, 47.5 mmol, 81% yield) as a white solid on drying under vacuum at 60° C.: LC/MS (Method g) Rₜ=1.45 min.; MS m/z: 188.00 (M+H)⁺, 1H NMR (400 MHz, CDCl3) δ ppm 12.71 (s, 1H), 8.06-8.03 (m, 1H), 8.00-7.98 (m, 1H), 3.76 (s, 3H)

### Step 2

### Preparation of methyl 5-chloro-6-isopropoxynicotinate

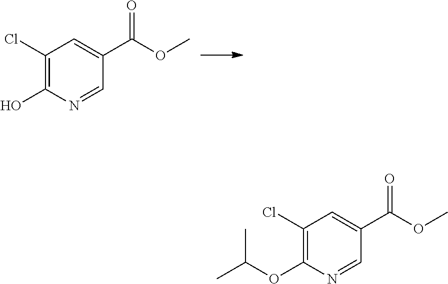

Methyl 5-chloro-6-hydroxynicotinate (8.91 g, 47.5 mmol) and 2-iodo-propane (7.12 mL, 71.2 mmol) were combined in toluene (202 mL) under nitrogen to give a colorless solution. Silver carbonate (19.65 g, 71.2 mmol) was added and the reaction heated at about 60° C. for about 4 h. TLC in 1:1 EtOAc/heptane showed (uv light visualization) that the reaction was not yet complete. The temperature was reduced to about 50° C. and the reaction stirred for about an additional 16 h. The reaction was allowed to cool to ambient temperature. The mixture was filtered through a buchner funnel and washed through with ethyl acetate. The solvents were removed under reduced pressure. The residue was purified by flash column chromatography (120 g Redi-Sep column) eluting with 5% ethyl acetate/heptane and the product fractions combined. The solvents were removed under reduced pressure to provide methyl 5-chloro-6-isopropoxynicotinate (10.539 g, 45.9 mmol, 97% yield) as a colorless oil that solidified under vacuum to a white solid: LC/MS (Method g) Rₜ=2.84 min.; MS m/z: 230.05 (M+H)⁺; 1H NMR (400 MHz, CDCl3) δ ppm 8.71 (d, J=2.02 Hz, 1H), 8.22 (d, J=1.93 Hz, 1H), 5.53-5.42 (m, 1H), 3.94 (s, 3H), 1.44 (d, J=6.20 Hz, 6H).

### Step 3

### Preparation of 5-chloro-6-isopropoxynicotinamide

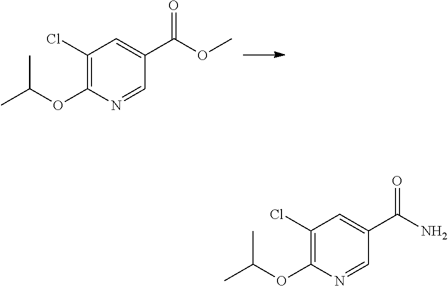

Methyl 5-chloro-6-isopropoxynicotinate (5.19 g, 22.60 mmol) was stirred in ammonia (7 M in methanol) (75 mL, 525 mmol) in a pressure vessel (with relief valve) to give a white suspension. The mixture was heated at about 60° C. for about 48 h. The reaction was cooled to about 0-5° C. in an ice bath and the reaction vessel opened. The solvents were removed under reduced pressure. The resulting white solid was taken up in water (100 mL) and stirred rapidly for about 2 h. The resulting solid was collected by vacuum filtration and washed with water and then pentane to provide 5-chloro-6-isopropoxynicotinamide (4.45 g, 20.73 mmol, 92% yield) as a white solid: LC/MS (Method g) Rₜ=1.91 min.; MS m/z: 215.04 (M+H)⁺, NMR (400 MHz,) δ 8.58 (d, J=2.2, 1H), 8.24 (d, J=2.2, 1H), 8.00 (s, 1H), 7.48 (s, 1H), 5.42-5.27 (m, 1H), 1.32 (d, J=6.2, 6H).

### Step 4

### Preparation of 5-chloro-6-isopropoxynicotinonitrile

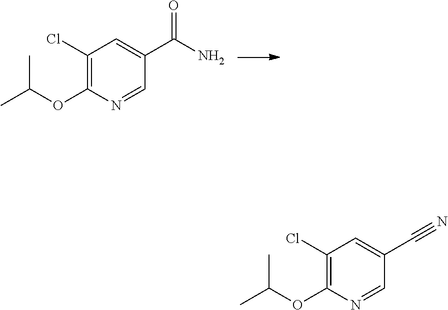

5-Chloro-6-isopropoxynicotinamide (6 g, 28.0 mmol) was dissolved in 1,2-dichloroethane (140 mL) under nitrogen to give a white suspension. Burgess reagent (13.32 g, 55.9 mmol) (Alfa Aesar) was added and the reaction stirred for about 16 h. As the reaction progressed all the solids dissolved (within an hour or two). The solvents were removed under reduced pressure. The residue was purified by flash column chromatography (80 g Redi-Sep column) eluting with 10% ethyl acetate/heptane and the product fractions combined. The solvents were removed under reduced pressure to provide 5-chloro-6-isopropoxynicotinonitrile (5.36 g, 27.3 mmol, 98% yield) as a white solid: LC/MS (Method g) Rₜ=2.60 min.; MS m/z: 196.07 (M+H)⁺, 1H NMR (400 MHz, DMSO) δ ppm 8.63 (d, J=2.03 Hz, 1H), 8.45 (d, J=2.04 Hz, 1H), 5.41-5.31 (m, 1H), 1.33 (d, J=6.17 Hz, 6H)

### Step 5

### Preparation of (Z)-5-chloro-N′-hydroxy-6-isopropoxynicotinimidamide

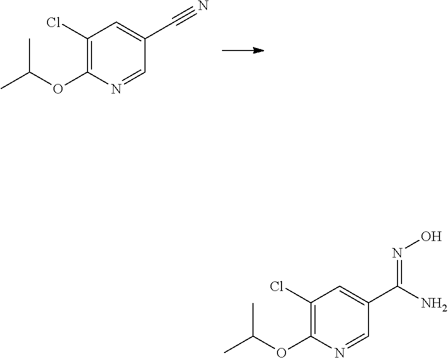

5-Chloro-6-isopropoxynicotinonitrile (5.36 g, 27.3 mmol) was dissolved in ethanol (136 mL) under nitrogen to give a colorless solution. Hydroxylamine (50% in water) (6.43 mL, 109 mmol) was added and the reaction heated at about 60° C. for about 3 h. TLC in 1:1 EtOAc/heptane showed (uv light visualization) that the reaction was complete. The reaction was concentrated under vacuum to a thick, colorless oil. Heptane was added to give a biphasic system. The oil was seeded with previously made product and crystallization formed a heavy precipitate. The precipitate was collected by vacuum filtration and washed with heptane. The mother liquor contained more product that precipitated. The mother liquor was concentrated, heptane added, and seeded to give a second crop. The crops looked the same by TLC and were combined to give (Z)-5-chloro-N′-hydroxy-6-isopropoxynicotinimidamide (6.108 g, 26.6 mmol, 98% yield) as a white solid on drying under vacuum at about 60° C. over phosphorous pentoxide: LC/MS (Method g) Rₜ=1.89 min.; MS m/z: 230.05 (M+H)⁺, 1H NMR (400 MHz, DMSO) δ=9.71 (s, 1H), 8.36 (d, J=2.1, 1H), 8.02 (d, J=2.1, 1H), 5.91 (s, 2H), 5.37-5.24 (m, 1H), 1.31 (d, J=6.2, 6H).

### Example #89

### 2-(4-(3-(4-isopropoxy-3-(trifluoromethyl)phenyl)-1,2,4-oxadiazol-5-yl)phenylamino)propan-1-ol

### Step 1

### Preparation of N-(1-(benzyloxy)propan-2-yl)-4-(3-(4-isopropoxy-3-(trifluoromethyl)phenyl)-1,2,4-oxadiazol-5-yl)aniline

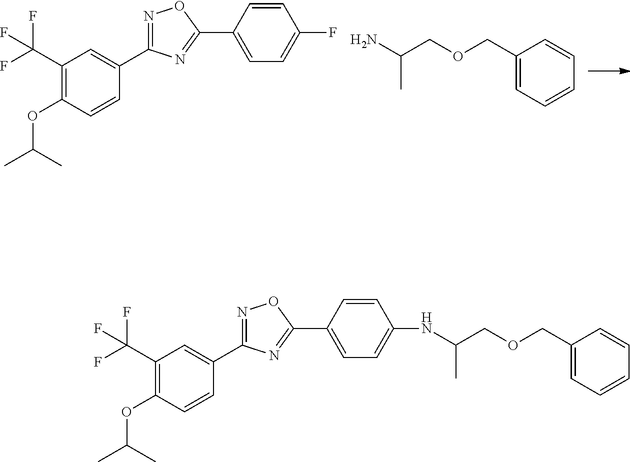

5-(4-Fluorophenyl)-3-(4-isopropoxy-3-(trifluoromethyl)phenyl)-1,2,4-oxadiazole (1.794 g, 4.90 mmol), 1-(benzyloxy)propan-2-amine (0.890 g, 5.39 mmol), and potassium carbonate (1.489 g, 10.77 mmol) were combined in DMSO (17 mL) and water (1.4 mL) under nitrogen to give a white suspension. The mixture was heated at about 110° C. for about 72 h. TLC in 1:1 EtOAc/heptane showed (uv light visualization) slow conversion to a more polar spot. The reaction was allowed to cool to ambient temperature. Ethyl acetate (75 mL) and water (50 mL) were added and the layers separated. The ethyl acetate was washed with water (2×25 mL). The combined extracts were washed with brine, dried over sodium sulfate, filtered, and evaporated to an off-white solid. This mixture was transferred to a microwave vial and 2 equivalents of the amine added (1.9 g). DMSO (15 mL) and water (1.2 mL) were added and the vial capped. The reaction was heated in a Biotage microwave (heating with cooling) at about 180° C. for about 30 minutes. TLC showed better progress toward product. Heating for about an additional 30 minutes gave approximately 50% conversion as judged by TLC. The reaction was heated for about an additional 60 minutes. The reaction was poured into water (50 mL) and ethyl acetate (150 mL). The ethyl acetate was washed with water (2×50 mL). The ethyl acetate was washed with brine, dried over sodium sulfate, filtered, and evaporated to a brown oil. With the aid of a small amount of methylene chloride the oil was transferred onto a column and was purified by flash column chromatography (80 g Redi-Sep column) eluting with 10-40% ethyl acetate/heptane and the product fractions combined. The solvents were removed under reduced pressure to provide N-(1-(benzyloxy)propan-2-yl)-4-(3-(4-isopropoxy-3-(trifluoromethyl)phenyl)-1,2,4-oxadiazol-5-yl)aniline (1.475 g, 2.88 mmol, 58.9% yield) as an orange oil: LC/MS (Method g) Rₜ=3.75 min.; MS m/z: 512.14 (M+H)⁺; 1H NMR (400 MHz, CDCl3) δ=8.39 (s, 1H), 8.27 (d, J=8.7, 1H), 8.02 (d, J=8.6, 2H), 7.43-7.30 (m, 5H), 7.12 (d, J=8.8, 1H), 6.68 (d, J=8.7, 2H), 4.82-4.70 (m, 1H), 4.59 (s, 2H), 4.43 (d, J=8.0, 1H), 3.88-3.76 (m, 1H), 3.57 (d, J=4.6, 2H), 1.44 (d, J=6.0, 6H), 1.33 (d, J=6.5, 3H).

### Step 2

### Preparation of 2-(4-(3-(4-isopropoxy-3-(trifluoromethyl)phenyl)-1,2,4-oxadiazol-5-yl)phenylamino)propan-1-ol

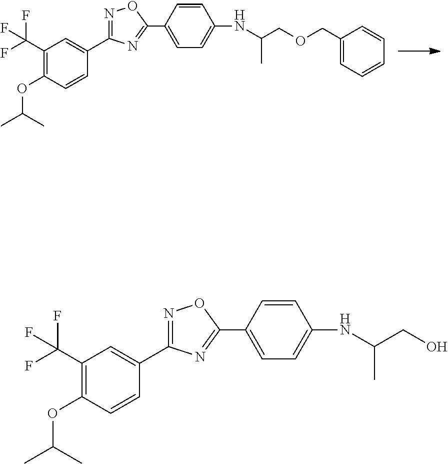

N-(1-(benzyloxy)propan-2-yl)-4-(3-(4-isopropoxy-3-(trifluoromethyl)phenyl)-1,2,4-oxadiazol-5-yl)aniline (1.111 g, 2.172 mmol) was dissolved in dichloromethane (145 mL) under nitrogen to give a colorless solution. The reaction was cooled to about 0-5° C. in an ice bath. A solution of boron tribromide (1.0 M in dichloromethane) (4.34 mL, 4.34 mmol) was added slowly and the reaction stirred for about 30 min. TLC in 1:1 EtOAc/heptane showed (uv light visualization) reaction complete having formed a more polar spot. The reaction was quenched by addition of 1 N HCl (10 mL). The cooling was removed and the reaction stirred for about 30 min. Saturated sodium bicarbonate (30 mL) was added and the layers separated. The aqueous layer was extracted with methylene chloride (1×30 mL). The combined extracts were washed with brine, dried over sodium sulfate, filtered, and evaporated to a yellow/tan solid. The residue (dissolved in methylene chloride) was purified by flash column chromatography (40 g Redi-Sep column) eluting with 20-60% ethyl acetate/heptane and the product fractions combined. The solvents were removed under reduced pressure. Pentane was added and the solid scraped from the sides of the flask. The resulting solid was collected by vacuum filtration and washed with pentane to provide 2-(4-(3-(4-isopropoxy-3-(trifluoromethyl)phenyl)-1,2,4-oxadiazol-5-yl)phenylamino)propan-1-ol (0.771 g, 1.830 mmol, 84% yield) as a white solid on drying under vacuum at about 70° C. over phosphorous pentoxide: LC/MS (Method g) Rₜ=2.95 min.; MS m/z: 422.14 (M+H)⁺; ¹H NMR (400 MHz, DMSO) δ=8.24 (dd, J=2.1, 8.8, 1H), 8.17 (d, J=2.1, 1H), 7.85 (d, J=8.8, 2H), 7.49 (d, J=8.9, 1H), 6.73 (d, J=8.9, 2H), 6.52 (d, J=7.9, 1H), 4.97-4.81 (m, 1H), 4.76 (t, J=5.6, 1H), 3.62-3.49 (m, 1H), 3.49-3.40 (m, 1H), 3.36-3.30 (m, 1H), 1.32 (d, J=6.0, 6H), 1.14 (d, J=6.4, 3H).

### Example #90

### (R)-2-(4-(3-(4-isopropoxy-3-(trifluoromethyl)phenyl)-1,2,4-oxadiazol-5-yl) phenoxy)propanoic acid

### Step 1

### Preparation of (R)-benzyl 4-(1-ethoxy-1-oxopropan-2-yloxy)benzoate

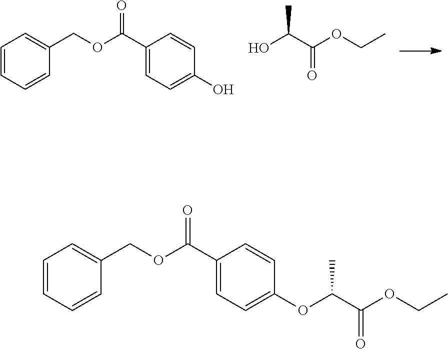

Benzyl 4-hydroxybenzoate (10 g, 43.8 mmol), (S)-ethyl 2-hydroxypropanoate (6.21 g, 52.6 mmol), and triphenylphosphine (14.36 g, 54.8 mmol) were combined in tetrahydrofuran (110 mL) under nitrogen to give a colorless solution. Molecular sieves 4 Å beads (4-8 mesh) (5 g) were added and the reaction stirred for about 30 min. The reaction was cooled to about 0-5° C. in an ice bath. Di-tertbutylazodicarboxylate (12.61 g, 54.8 mmol) was added (exotherm observed) and the reaction stirred for about 18 h. TLC in 1:1 EtOAc/heptane showed (uv light visualization) that the reaction was complete. The mixture was filtered through Celite® and washed through with ethyl acetate. The solvents were removed under reduced pressure. Heptane was added and the sides scraped to induce crystallization of triphenylphosphine oxide and reduced di-tertbutylazodicarboxylate. The flask was swirled until a thick precipitate had formed. After standing overnight the solids were removed by vacuum filtration and the solid washed with heptane until no further product eluted as judged by TLC. The solvents were removed under reduced pressure. The residue was purified by flash column chromatography (120 g Redi-Sep column) eluting with 10-20% ethyl acetate/heptane and the product fractions combined. The product fractions were concentrated to provide (R)-benzyl 4-(1-ethoxy-1-oxopropan-2-yloxy)benzoate (16.3 g, 49.6 mmol, 113% yield) as a colorless oil: LC/MS (Method g) Rₜ=2.83 min.; MS m/z: 329.15 (M+H)⁺; 1H NMR (400 MHz, CDCl3) δ 8.05 (d, J=8.9, 2H), 7.60-7.21 (m, 5H), 6.91 (d, J=8.8, 2H), 5.36 (s, 2H), 4.84 (q, J=6.8, 1H), 4.24 (q, J=7.1, 2H), 1.67 (d, J=6.8, 3H), 1.27 (t, J=7.1, 3H).

### Step 2

### Preparation of (R)-4-(1-ethoxy-1-oxopropan-2-yloxy)benzoic acid

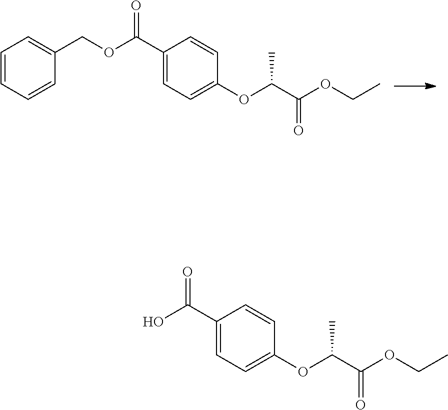

(R)-benzyl 4-(1-ethoxy-1-oxopropan-2-yloxy)benzoate (14.39 g, 43.8 mmol) was dissolved in methanol (438 mL). Palladium hydroxide on carbon (20%) (3.08 g, 4.38 mmol) was added, the reaction was flushed with hydrogen, and hydrogenated at atmospheric pressure for about 3 h. TLC in 1:1 EtOAc/heptane showed (uv light visualization) that the reaction was complete. The reaction was flushed with nitrogen. The mixture was filtered through Celite® and washed through with methanol. The solvents were removed under reduced pressure. Ethyl acetate was added and the solvents removed again to remove any remaining methanol providing a white solid. Heptane was added and the solid scraped from the sides of the flask. The resulting solid was collected by vacuum filtration and washed with heptane to provide (R)-4-(1-ethoxy-1-oxopropan-2-yloxy)benzoic acid (8.83 g, 37.1 mmol, 85% yield) as a white solid:

LC/MS (Method g) Rₜ=1.88 min.; MS m/z: 237.08 (M+H)⁺, 1H NMR (400 MHz, DMSO) δ 12.67 (m, 1H), 7.85 (d, J=8.9, 2H), 6.94 (d, J=8.9, 2H), 5.06 (d, J=6.8, 1H), 4.13 (q, J=7.1, 2H), 1.51 (d, J=6.8, 3H), 1.15 (t, J=7.1, 3H).

### Step 3

### Preparation of (R)-ethyl 2-(4-(3-(4-isopropoxy-3-(trifluoromethyl)phenyl)-1,2,4-oxadiazol-5-yl)phenoxy)propanoate

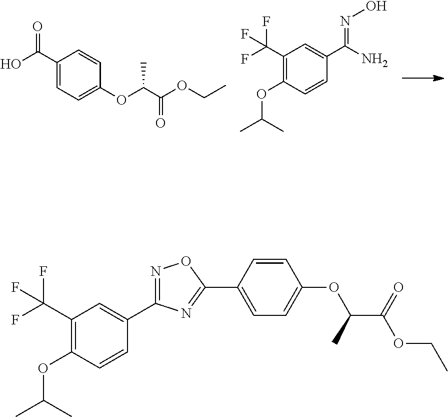

(R)-4-(1-ethoxy-1-oxopropan-2-yloxy)benzoic acid (1 g, 4.20 mmol) and HOBt (0.884 g, 4.62 mmol) were combined in methylene chloride (20.99 mL) under nitrogen to give a white suspension. EDC (0.885 g, 4.62 mmol) was added and the reaction stirred for about 4 h. TLC in 1:1 EtOAc/heptane showed (uv light visualization) a less polar spot had formed. Methylene chloride (20 mL) and water (20 mL) were added and the layers separated. The methylene chloride was washed with water (2×15 mL). The combined extracts were washed with brine, dried over sodium sulfate, filtered, and evaporated to a white solid. The solid was dissolved in DMF (20 mL) and (Z)—N′-hydroxy-4-isopropoxy-3-(trifluoromethyl)benzimidamide (1.101 g, 4.20 mmol) was added. The reaction was heated at about 60° C. for about 72 h. TLC showed formation of an intermediate spot and a non-polar spot. LC/MS showed this to be the uncyclized intermediate and the oxadiazole product. The reaction temperature was increased to about 90° C. and the reaction progressed more rapidly. A further increase to about 110° C. for about 4 h completed the reaction. The reaction was cooled to ambient temperature and poured into water (about 200 mL). The aqueous mixture was extracted with ethyl acetate (3×50 mL). The combined ethyl acetate extracts were washed with 5% lithium chloride solution (3×30 mL). The ethyl acetate was then washed with brine, dried over sodium sulfate, filtered and evaporated. The resulting residue was purified by flash column chromatography (80 g Redi-sep column) eluting with ethyl acetate/heptane and the product fractions combined. The solvents were removed under reduced pressure to provide (R)-ethyl 2-(4-(3-(4-isopropoxy-3-(trifluoromethyl)phenyl)-1,2,4-oxadiazol-5-yl)phenoxy)propanoate (1.373 g, 2.96 mmol, 70.4%) as an off white solid:

LC/MS (Method g) Rₜ=3.50 min.; MS m/z: 465.15 (M+H)⁺, ¹H NMR (400 MHz, CDCl3) δ 8.40 (s, 1H), 8.28 (d, J=8.7, 1H), 8.18 (d, J=7.7, 2H), 7.13 (d, J=8.6, 1H), 7.04 (d, J=7.8, 2H), 4.89 (dd, J=6.7, 13.5, 1H), 4.83-4.70 (m, 1H), 4.28 (q, J=7.1, 2H), 1.71 (d, J=5.8, 3H), 1.45 (d, J=4.9, 6H), 1.30 (t, J=7.1, 3H).

### Step 4

### Preparation of (R)-2-(4-(3-(4-isopropoxy-3-(trifluoromethyl)phenyl)-1,2,4-oxadiazol-5-yl)phenoxy)propanoic acid

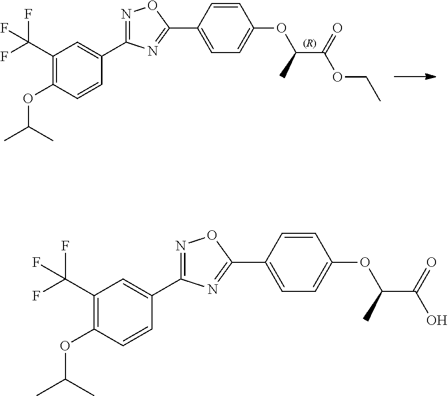

(R)-ethyl 2-(4-(3-(4-isopropoxy-3-(trifluoromethyl)phenyl)-1,2,4-oxadiazol-5-yl)phenoxy)propanoate (0.5 g, 1.077 mmol) was dissolved in ethanol (10 mL) open to the air to give a white suspension. Sodium hydroxide (1 M) (2.153 mL, 2.153 mmol) was added and the reaction stirred for about 2 h. The starting material was not very soluble. Tetrahydrofuran (5 mL) was added and the reaction sonicated. All the material dissolved and the reaction stirred for about an additional 30 min. TLC in 1:1 EtOAc/heptane showed (uv light visualization) that the reaction was complete. The solvents were removed under reduced pressure. 1 N HCl (2.5 mL) was added and the aqueous layer was extracted with ethyl acetate (2×10 mL). The combined extracts were washed with brine, dried over sodium sulfate, filtered, and evaporated to a white solid. The residue was purified by flash column chromatography (40 g Redi-Sep column) eluting with 5-10% methanol in methylene chloride and the product fractions combined. The solvents were removed under reduced pressure to provide (R)-2-(4-(3-(4-isopropoxy-3-(trifluoromethyl)phenyl)-1,2,4-oxadiazol-5-yl) phenoxy)propanoic acid (0.286 g, 0.655 mmol, 60.9% yield) as a white solid on drying under vacuum at about 60° C.: LC/MS (Method g) Rₜ=2.31 min.; MS m/z: 437.11 (M+H)⁺; 1H NMR (400 MHz, DMSO) δ 13.16 (s, 0H), 8.26 (dd, J=2.1, 8.8, 1H), 8.19 (d, J=2.1, 1H), 8.11 (d, J=8.9, 2H), 7.51 (d, J=8.9, 1H), 7.11 (d, J=9.0, 2H), 5.02 (q, J=6.7, 1H), 4.96-4.85 (m, 1H), 1.54 (d, J=6.8, 3H), 1.32 (d, J=6.0, 6H).

### Example #91

### (R)—N-(2-hydroxyethyl)-2-(4-(3-(4-isopropoxy-3-(trifluoromethyl)phenyl)-1,2,4-oxadiazol-5-yl) phenoxy)propanamide

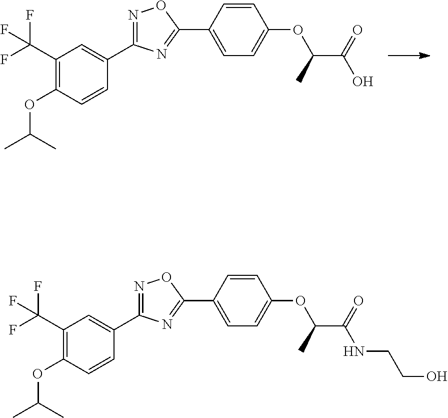

(R)-2-(4-(3-(4-isopropoxy-3-(trifluoromethyl)phenyl)-1,2,4-oxadiazol-5-yl)phenoxy)propanoic acid (0.05 g, 0.115 mmol) and HOBT (0.021 g, 0.115 mmol) were combined in dichloromethane (2.29 mL) in a sealed vial to give a colorless solution. EDC (0.022 g, 0.115 mmol) was added and the reaction stirred for about 30 min. Ethanolamine (30 μl, 0.496 mmol) was added and the reaction stirred for about 30 min. TLC in EtOAc showed (uv light visualization) that the reaction was complete. Methylene chloride (5 mL) and water (5 mL) were added and the layers separated. The methylene chloride was washed with 1 N HCl (1×5 mL). The methylene chloride was washed with brine, dried over sodium sulfate, filtered, and evaporated to a colorless oil. The residue was purified by flash column chromatography (40 g Redi-Sep column) eluting with 50-80% ethyl acetate/heptane and the product fractions combined. The solvents were removed under reduced pressure. The residue was triturated with pentane and the resulting solid was collected by vacuum filtration and washed with pentane to provide (R)—N-(2-hydroxyethyl)-2-(4-(3-(4-isopropoxy-3-(trifluoromethyl)phenyl)-1,2,4-oxadiazol-5-yl)phenoxy)propanamide (0.043 g, 0.090 mmol, 78% yield) as a white solid: LC/MS (Method g) Rₜ=2.64 min.; MS m/z: 480.17 (M+H)⁺; 1H NMR (400 MHz, DMSO) δ 8.27 (dd, J=2.1, 8.8, 1H), 8.22-8.18 (m, 1H), 8.15 (t, J=5.9, 1H), 8.13-8.09 (m, 1H), 7.56-7.46 (m, 1H), 7.19-7.11 (m, 2H), 4.96-4.84 (m, 2H), 4.67 (q, J=5.4, 1H), 3.42-3.33 (m, 2H), 3.20-3.11 (m, 2H), 1.46 (d, J=6.6, 3H), 1.32 (d, J=6.0, 6H).

### Example #92

### (1R,3S)-3-(4-(5-(5-bromo-6-isopropoxypyridin-3-yl)-1,2,4-oxadiazol-3-yl)phenylamino)cyclopentanecarboxylic acid

### Step 1

### Preparation of methyl 5-bromo-6-hydroxynicotinate

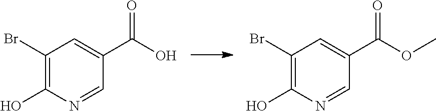

5-Bromo-6-hydroxynicotinic acid (4.9 g, 22.48 mmol) (Combi-Blocks) was stirred in methanol (39.0 mL) under nitrogen to give a off-white suspension. Sulfuric acid (5.99 mL, 112 mmol) was added slowly and the reaction heated at about 60° C. for about 20 h. The reaction was allowed to cool to ambient temperature. The resulting solid was collected by vacuum filtration and washed with methanol and then ether to provide methyl 5-bromo-6-hydroxynicotinate (4.041 g, 17.42 mmol, 77% yield) as a off-white solid on drying under vacuum at 50° C.: LC/MS (Method g) Rₜ=1.55 min.; MS m/z: 233.95 (M+H)⁺; ¹H NMR (400 MHz, DMSO) δ 12.66 (s, 1H), 8.16 (d, J=2.4, 1H), 8.07 (d, J=2.4, 1H), 3.76 (s, 3H).

### Step 2

### Preparation of methyl 5-bromo-6-isopropoxynicotinate

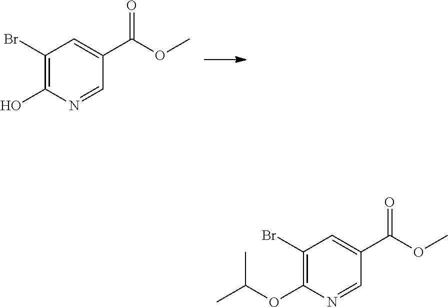

Methyl 5-bromo-6-hydroxynicotinate (4 g, 17.24 mmol) and 2-iodopropane (2.58 mL, 25.9 mmol) (Alfa Aesar) were combined in toluene (73 mL) and equipped with a reflux condenser under nitrogen to give a white suspension. Silver carbonate (7.13 g, 25.9 mmol) (Alfa Aesar) was added and the reaction heated at about 60° C. for about 24 h. The mixture was filtered through Celite® and washed through with ethyl acetate until no further product came out as judged by TLC. The solvents were removed under reduced pressure to provide methyl 5-bromo-6-isopropoxynicotinate (4.809 g, 17.54 mmol, 102% yield) as a white solid: LC/MS (Method g) Rₜ=2.92 min.; MS m/z: 274.00, 276.00 (M+H)⁺, 1H NMR (400 MHz, DMSO) δ 8.67 (d, J=2.1, 1H), 8.35 (d, J=2.1, 1H), 5.42-5.30 (m, 1H), 3.83 (s, 3H), 1.33 (d, J=6.2, 6H).

### Step 3

### Preparation of 5-bromo-6-isopropoxynicotinic acid

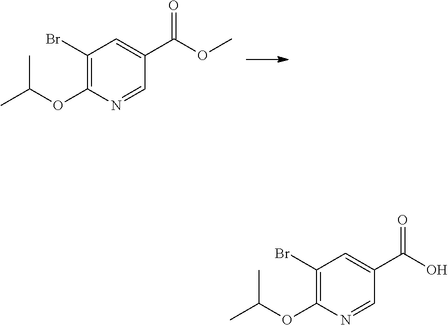

Methyl 5-bromo-6-isopropoxynicotinate (4.8 g, 17.51 mmol) and sodium hydroxide (1.401 g, 35.0 mmol) were combined in methanol (31 mL) and water (3.5 mL) under nitrogen to give a cloudy colorless solution. The mixture was heated at about 50° C. for about 6 h. The reaction was allowed to cool to ambient temperature. The solvents were removed under reduced pressure. The residue was dissolved in water. The mixture was acidified to about pH=1 with 1 N HCl causing a thick white precipitate to form. The resulting solid was collected by vacuum filtration and washed with water to provide 5-bromo-6-isopropoxynicotinic acid (4.155 g, 15.98 mmol, 91% yield) as a white solid on drying under vacuum at about 50° C. over phosphorous pentoxide: LC/MS (Method g) Rₜ=1.92 min.; MS m/z: 257.98, 259.97 (M−H)⁻; 1H NMR (400 MHz, DMSO) δ 13.26 (s, 1H), 8.65 (d, J=2.0, 1H), 8.31 (d, J=2.1, 1H), 5.35 (dt, J=6.2, 12.4, 1H), 1.33 (d, J=6.2, 6H).

### Step 4

### Preparation of (1R,3S)-ethyl 3-(4-(5-(5-bromo-6-isopropoxypyridin-3-yl)-1,2,4-oxadiazol-3-yl)phenylamino)cyclopentanecarboxylate

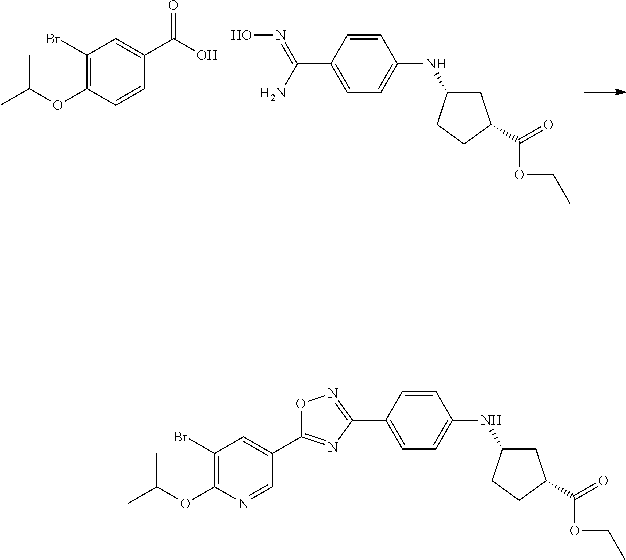

5-Bromo-6-isopropoxynicotinic acid (0.1 g, 0.384 mmol), HOBt (0.081 g, 0.423 mmol), and molecular sieves 4 Å (4-8 mesh beads) (0.5 g) were combined in DMF (1.922 mL) under nitrogen to give a colorless suspension. EDC (0.081 g, 0.423 mmol) and N,N-Diisoproplylethylamine (0.074 mL, 0.423 mmol) were added and the reaction stirred for about min. (1R,3S)-ethyl 3-(4-((Z)—N′-hydroxycarbamimidoyl)phenylamino)cyclopentanecarboxylate (0.101 g, 0.346 mmol) was added and the reaction heated at about 100° C. for about 6 h. Ethyl acetate (15 mL) and water (10 mL) were added and the layers separated. The ethyl acetate was washed with water (2×5 mL). The combined extracts were washed with brine, dried over sodium sulfate, filtered, and evaporated to a brown oil. The residue was purified by flash column chromatography (40 g Redi-Sep) eluting with 10-20% ethyl acetate/heptane and the product fractions combined. The solvents were removed under reduced pressure to provide (1R,3S)-ethyl 3-(4-(5-(5-bromo-6-isopropoxypyridin-3-yl)-1,2,4-oxadiazol-3-yl)phenylamino)cyclopentanecarboxylate (0.023 g, 0.045 mmol, 11.6% yield): LC/MS (Method g) Rₜ=3.94 min.; MS m/z: 515.11, 517.11 (M+H)⁺, ¹H NMR (400 MHz, DMSO) δ 8.89 (d, J=2.1, 1H), 8.60 (d, J=2.1, 1H), 7.77 (d, J=8.6, 2H), 6.68 (d, J=8.7, 2H), 6.41 (d, J=6.7, 1H), 5.47-5.35 (m, 1H), 4.05 (dd, J=7.1, 14.2, 2H), 3.87-3.75 (m, 1H), 2.89-2.76 (m, 1H), 2.38-2.28 (m, 1H), 2.06-1.94 (m, 1H), 1.88 (dd, J=7.6, 15.2, 2H), 1.69-1.59 (m, 1H), 1.58-1.47 (m, 1H), 1.36 (d, J=6.2, 6H), 1.15 (t, J=7.1, 3H).

### Step 5

### Preparation of (1R,3S)-3-(4-(5-(5-bromo-6-isopropoxypyridin-3-yl)-1,2,4-oxadiazol-3-yl)phenylamino)cyclopentanecarboxylic acid

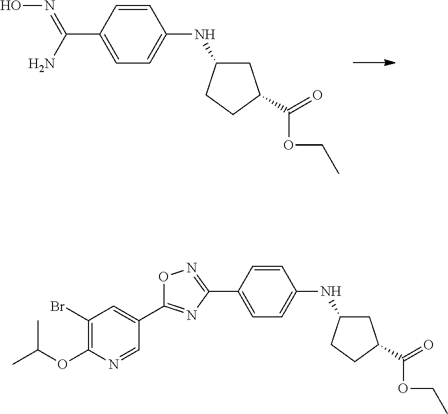

(1R,3S)-Ethyl 3-(4-(5-(5-bromo-6-isopropoxypyridin-3-yl)-1,2,4-oxadiazol-3-yl)phenylamino)cyclopentanecarboxylate (0.023 g, 0.045 mmol) was dissolved in ethanol (0.446 mL) in a sealed vial to give a white suspension. A solution of sodium hydroxide (1 M) (0.179 mL, 0.179 mmol) was added in one portion and the reaction stirred for about 4 h. THF (1 mL) was added and the reaction cleared. LC/MS indicated that the reaction was complete. The solvents were removed under reduced pressure. The residue was dissolved in water (about 3 mL) and 1 N HCl (0.2 mL) was added with rapid stirring causing a white precipitate to form. The mixture was stirred for about 1 h to ensure complete precipitation. The resulting solid was collected by vacuum filtration and washed with water and pentane to provide (1R,3S)-3-(4-(5-(5-bromo-6-isopropoxypyridin-3-yl)-1,2,4-oxadiazol-3-yl)phenylamino)cyclopentanecarboxylic acid (0.02 g, 0.041 mmol, 92% yield) as an off-white solid on drying under vacuum at 50° C. over phosphorous pentoxide: LC/MS (Method g) Rₜ=3.14 min.; MS m/z: 487.1 (M+H)⁺; 1H NMR (400 MHz, DMSO) δ 12.03 (s, 1H), 8.89 (d, J=2.1, 1H), 8.60 (d, J=2.1, 1H), 7.77 (d, J=8.6, 2H), 6.68 (d, J=8.7, 2H), 6.41 (d, J=6.6, 1H), 5.46-5.34 (m, 1H), 3.86-3.74 (m, 1H), 2.81-2.70 (m, 1H), 2.38-2.26 (m, 1H), 2.06-1.92 (m, 1H), 1.86 (dd, J=7.6, 15.2, 2H), 1.71-1.57 (m, 1H), 1.57-1.46 (m, 1H), 1.36 (d, J=6.2, 6H).

### Example #93

### Preparation of (R)-2-(4-(3-(4-isopropoxy-3-(trifluoromethyl)phenyl)-1,2,4-oxadiazol-5-yl)phenoxy)propanal

### Step 1

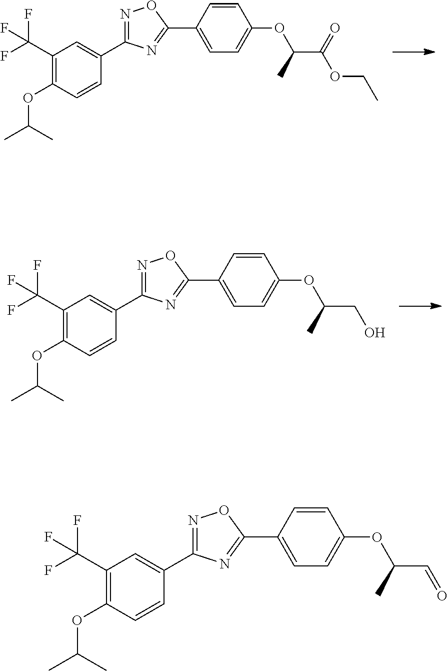

(R)-ethyl 2-(4-(3-(4-isopropoxy-3-(trifluoromethyl)phenyl)-1,2,4-oxadiazol-5-yl)phenoxy)propanoate (0.608 g, 1.309 mmol) was dissolved in tetrahydrofuran (13.09 mL) under nitrogen to give a colorless solution. The reaction was cooled to about 0-5° C. in an ice bath. A solution of lithium aluminum hydride (2 M in THF) (1.309 mL, 2.62 mmol) was added dropwise and the reaction stirred for about 30 min. TLC in 1:1 EtOAc/heptane showed (uv light visualization) conversion to a more polar spot. The reaction was quenched by addition of water (100 uL) and the reaction stirred for about 15 min. 10% NaOH (300 uL) was added and the reaction stirred for about 30 minutes. Water (100 μL) was added and the reaction stirred for about 30 min. The mixture was filtered through Celite® and washed through with ethyl acetate. The solution was then dried over sodium sulfate, filtered and concentrated. The residue was dissolved in dichloromethane (13.09 mL). Dess-Martin periodinane (1.111 g, 2.62 mmol) was added and the reaction stirred for about 5 h. The reaction was quenched by addition of saturated sodium bicarbonate (1 mL) and methylene chloride (5 mL). The layers were separated. The aqueous layer was extracted with methylene chloride (1×2 mL). The combined extracts were washed with brine, dried over sodium sulfate, filtered, and evaporated to a colorless oil. The residue was purified by flash column chromatography (40 g Redi-Sep column) eluting with 20-40% ethyl acetate/heptane and the product fractions combined. The solvents were removed under reduced pressure to provide (R)-2-(4-(3-(4-isopropoxy-3-(trifluoromethyl)phenyl)-1,2,4-oxadiazol-5-yl)phenoxy)propanal (0.281 g, 0.668 mmol, 51.1% yield) as a off-white solid: LC/MS (Method g) Rₜ=2.77 min.; MS m/z: 439.14 (M+H2O)⁺; ¹H NMR (400 MHz, DMSO) δ 9.68 (s, 1H), 8.26 (dd, J=2.0, 8.8, 1H), 8.19 (d, J=2.0, 1H), 8.11 (d, J=8.8, 2H), 7.50 (d, J=8.9, 1H), 7.19 (d, J=8.8, 2H), 5.23 (q, J=7.0, 1H), 4.96-4.85 (m, 1H), 1.46 (d, J=7.0, 3H), 1.32 (d, J=6.0, 6H).

### Step 2

### Preparation of (R,E)-ethyl 4-(4-(3-(4-isopropoxy-3-(trifluoromethyl)phenyl)-1,2,4-oxadiazol-5-yl)phenoxy)-2-methylpent-2-enoate

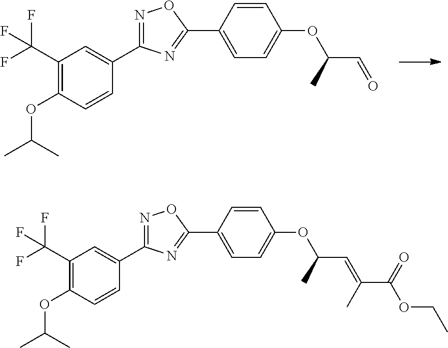

(R)-2-(4-(3-(4-isopropoxy-3-(trifluoromethyl)phenyl)-1,2,4-oxadiazol-5-yl)phenoxy)propanal (0.281 g, 0.668 mmol) was dissolved in dichloromethane (6.68 mL) under nitrogen to give a colorless solution. Ethyl 2-(triphenylphosphoranylidene)propionate (0.266 g, 0.735 mmol) was added and the reaction stirred for about 18 h. The solvents were removed under reduced pressure. The residue was purified by flash column chromatography (40 g Redi-Sep column) eluting with 20-40% ethyl acetate/heptane and the product fractions combined. The solvents were removed under reduced pressure to provide (R,E)-ethyl 4-(4-(3-(4-isopropoxy-3-(trifluoromethyl)phenyl)-1,2,4-oxadiazol-5-yl)phenoxy)-2-methylpent-2-enoate (0.281 g, 0.557 mmol, 83% yield) as a white solid: LC/MS (Method g) Rₜ=3.93 min.; MS m/z: 505.17 (M+H)⁺; ¹H NMR (400 MHz, DMSO) δ 8.26 (dd, J=2.1, 8.7, 1H), 8.19 (d, J=2.0, 1H), 8.11 (d, J=8.8, 2H), 7.50 (d, J=8.9, 1H), 7.11 (d, J=8.9, 2H), 6.56 (dd, J=1.3, 7.9, 1H), 5.52-5.42 (m, 1H), 4.96-4.84 (m, 1H), 4.10 (qd, J=1.6, 7.1, 2H), 1.94 (d, J=1.0, 3H), 1.42 (d, J=6.3, 3H), 1.32 (d, J=6.0, 6H), 1.19 (t, J=7.1, 3H).

### Step 3

### Preparation of (R,E)-ethyl 4-(4-(3-(4-isopropoxy-3-(trifluoromethyl)phenyl)-1,2,4-oxadiazol-5-yl)phenoxy)-2-methylpent-2-enoate

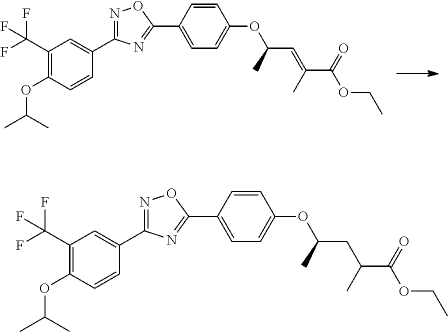

(R,E)-ethyl 4-(4-(3-(4-isopropoxy-3-(trifluoromethyl)phenyl)-1,2,4-oxadiazol-5-yl)phenoxy)-2-methylpent-2-enoate (0.281 g, 0.557 mmol) was dissolved in ethyl acetate (11.14 mL). Palladium on carbon (10%) (0.059 g, 0.056 mmol) was added, the reaction was flushed with hydrogen, and hydrogenated at atmospheric pressure for about 4 h. LC/MS showed product and oxadiazole opening. The mixture was filtered through a syringe filter and washed through with ethyl acetate. The solvents were removed under reduced pressure. The residue was purified by flash column chromatography (40 g Redi-Sep column) eluting with 20-40% ethyl acetate/heptane and the product fractions combined. The solvents were removed under reduced pressure to provide (4R)-ethyl 4-(4-(3-(4-isopropoxy-3-(trifluoromethyl)phenyl)-1,2,4-oxadiazol-5-yl)phenoxy)-2-methylpentanoate (0.105 g, 0.207 mmol, 37.2% yield) as a colorless oil: LC/MS (Method i) Rₜ=2.17 min.; MS m/z: 507.18 (M+H)⁺, ¹H NMR (400 MHz, DMSO) δ 8.26 (dd, J=2.1, 8.8, 1H), 8.19 (d, J=2.0, 1H), 8.09 (d, J=8.9, 2H), 7.50 (d, J=8.9, 1H), 7.12 (d, J=8.9, 2H),4.95-4.84 (m, 1H), 4.69-4.56 (m, 1H), 4.10-3.92 (m, 2H), 2.68-2.50 (m, 2H), 2.11-2.02 (m, 1H), 1.97-1.76 (m, 2H), 1.69-1.61 (m, 1H), 1.32 (d, J=6.0, 6H), 1.26 (d, J=6.0, 3H), 1.15-1.01 (m, 3H) major diastereomer, minor diastereomer observed at δ8.10 (d, J=9.0, 2H), 7.14 (d, J=8.9, 2H).

### Step 4

### Preparation of (R)-4-(4-(3-(4-isopropoxy-3-(trifluoromethyl)phenyl)-1,2,4-oxadiazol-5-yl)phenoxy)-2-methylpentanoic acid

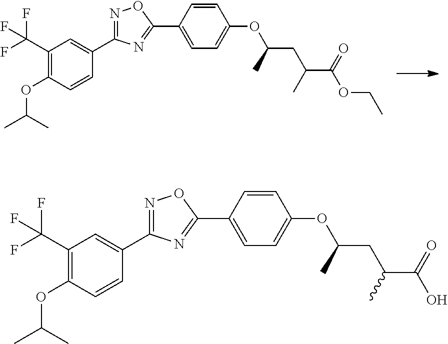

(4R)-ethyl 4-(4-(3-(4-isopropoxy-3-(trifluoromethyl)phenyl)-1,2,4-oxadiazol-5-yl)phenoxy)-2-methylpentanoate (0.105 g, 0.207 mmol) was dissolved in ethanol (2.073 mL) under nitrogen to give a colorless solution. A solution of sodium hydroxide (1 M) (0.829 mL, 0.829 mmol) was added in one portion and the reaction stirred for about 20 h. On addition of the sodium hydroxide the reaction turned cloudy but it became homogeneous after reacting overnight. The solvents were removed under reduced pressure. The residue was dissolved in water (about 5 mL) with sonication. 1 N HCl (900 uL) was added to bring the mixture to about pH=1. A white precipitate formed and was stirred for about 2 h. The resulting solid was collected by vacuum filtration and washed with water and a small amount of pentane to provide (R)-4-(4-(3-(4-isopropoxy-3-(trifluoromethyl)phenyl)-1,2,4-oxadiazol-5-yl)phenoxy)-2-methylpentanoic acid (0.081 g, 0.169 mmol, 82% yield) as a white solid: LC/MS (Method g) Rₜ=3.24 min.; MS m/z: 479.16 (M+H)⁺ (major diastereomer) and Rₜ=3.20 min.; MS m/z: 479.16 (M+H)⁺ (minor diastereomer), ¹H NMR (400 MHz, DMSO) δ 12.10 (s, 1H), 8.27 (dd, J=1.9, 8.7, 1H), 8.19 (d, J=1.9, 1H), 8.10 (d, J=8.8, 2H), 7.51 (d, J=8.9, 1H), 7.19-7.09 (m, 2H), 4.97-4.84 (m, 1H), 4.69-4.57 (m, 1H), 2.59-2.50 (m, 1H), 1.97-1.84 (m, 1H), 1.75 (d, J=19.4, 1H), 1.33 (d, J=6.0, 6H), 1.27 (d, J=6.0, 3H), 1.10 (d, J=7.1, 3H). Minor diastereomer observed at 2.13-2.02 (m, 1H), 1.66-1.53 (m, 1H), 1.31 (d, J=6.0, 6H), 1.08 (d, J=7.1, 3H).

### Preparation #86

### Preparation of 5-chloro-6-isopropoxynicotinic acid

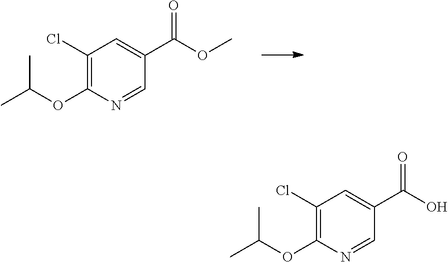

Methyl 5-chloro-6-isopropoxynicotinate (24 g, 105 mmol) was stirred in methanol (190 mL) and water (19.00 mL) under nitrogen to give a colorless solution. Sodium hydroxide (5.43 g, 136 mmol) was added and the reaction heated at about 60° C. for about 6 h. TLC in EtOAc showed (uv light visualization) that the reaction was complete. The reaction was allowed to cool to ambient temperature. The solvents were removed under reduced pressure. The white residue was redissolved in water (about 250 mL) to give a cloudy solution. The mixture was filtered through Celite and washed through with water (about 200 mL). The clear aqueous solution was acidified to about pH 2 with 1 N HCl (140 mL) causing a heavy white precipitate to form. The resulting solid was collected by vacuum filtration and washed with water and then pentane to provide 5-chloro-6-isopropoxynicotinic acid (21.946 g, 102 mmol, 97% yield) as a white solid on drying under vacuum at about 50° C. over phosphorous pentoxide: LC/MS (Table 1, Method g) Rₜ=1.91 min.; MS m/z: 214.02, 216.04 (M−H)⁻, ¹H NMR (400 MHz, DMSO) δ ppm 13.29 (s, 1H), 8.62 (d, J=2.0, 1H), 8.18 (d, J=2.0, 1H), 5.44-5.31 (m, 1H), 1.33 (d, J=6.2, 6H).

### Preparation #87

### 3-(3-Chloro-4-cyano-phenoxy)-cyclobutanecarboxylic acid tert-butyl ester

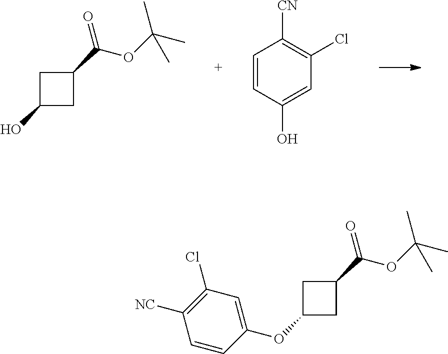

A solution of 2-chloro-4-hydroxybenzonitrile (5.00 g, 32.6 mmol) and (1s,3s)-tert-butyl 3-hydroxycyclobutanecarboxylate described in WO20070607 (6.17 g, 35.8 mmol) in THF (220 mL) is treated with 4 Å molecular sieves (10 g) and Ph₃P-Resin bound (32.6 mL, 98 mmol) for 20 min. at RT, then cooled to about 0° C. A solution of di-tert-butyl azodicarboxylate (8.25 g, 35.8 mmol) in THF (30 mL) is added dropwise while maintaining the reaction temperature below about 4° C. The reaction is stirred about 15 minutes at about 0° C. and then allowed to warm to RT for about 18 h. The reaction was filtered and the solids rinsed with methanol (3×50 mL) and the combined organic solutions were concentrated. The residue was purified on a silica gel column using a gradient from 10-30% ethyl acetate in heptane. The product fractions were combined and concentrated to a clear, colorless oil which solidified on standing to yield 3-(3-Chloro-4-cyano-phenoxy)-cyclobutanecarboxylic acid tert-butyl ester, (6.03 g, 60%) as a white solid. LC/MS (Table 1, Method a) Rₜ=2.75 min.; MS m/z: No parent mass. ¹H NMR (400 MHz, DMSO) δ ppm 7.87 (d, J=8.7, 1H), 7.19 (d, J=2.4, 1H), 6.99 (dd, J=8.7, 2.4, 1H), 4.99-4.92 (m, 1H), 3.11-3.04 (m, 1H), 2.69-2.63 (m, 2H), 2.40-2.28 (m, 2H), 1.44 (s, 9H).

### Preparation #88

### 3-[3-Chloro-4-(N-hydroxycarbamimidoyl)-phenoxy]cyclobutanecarboxylic acid tert-butyl ester

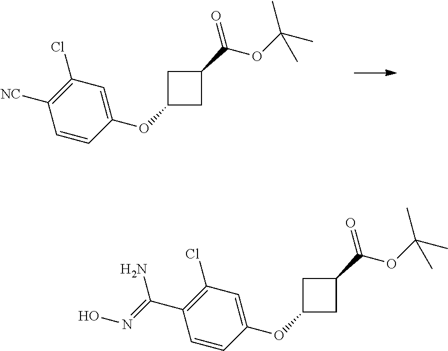

Into a round bottom flask was added (1r,3r)-tert-butyl 3-(3-chloro-4-cyanophenoxy)cyclobutanecarboxylate (5.98 g, 19.43 mmol), hydroxylamine (50% by weight in water, 6.88 mL, 117 mmol) and ethanol (50 mL). The mixture was heated at 60° C. overnight. Upon completion of the reaction, the mixture was cooled to room temperature, diluted with 150 mL water and extracted with 150 mL ethyl acetate. The organic layer was washed with saturated NaCl solution (100 mL), dried over sodium sulfate, filtered and concentrated to solids. The crude product was triturated with ether to obtain 3-[3-Chloro-4-(N-hydroxycarbamimidoyl)-phenoxy]-cyclobutanecarboxylic acid tert-butyl ester (5.43 g, 82%) as a white solid.

LC/MS (Table 1, Method a) Rₜ=2.15 min, m/z 341 (M+H)⁺; ¹H NMR (400 MHz, DMSO-d₆) δ ppm 9.37 (s, 1H), 7.30 (d, J=8.5, 1H), 6.89 (d, J=2.5, 2H), 6.81 (dd, J=8.5, 2.5, 1H), 5.71 (s, 2H), 4.91-4.81 (m, 1H), 3.12-3.01 (m, 1H), 2.63 (m, 2H), 2.37-2.26 (m, 2H), 1.44 (s, 9H).

### Preparation #89

### 3-{3-Chloro-4-[5-(5-chloro-6-isopropoxy-pyridin-3-yl)-[1,2,4]oxadiazol 3-yl]-phenoxy}-cyclobutanecarboxylic acid tert-butyl ester

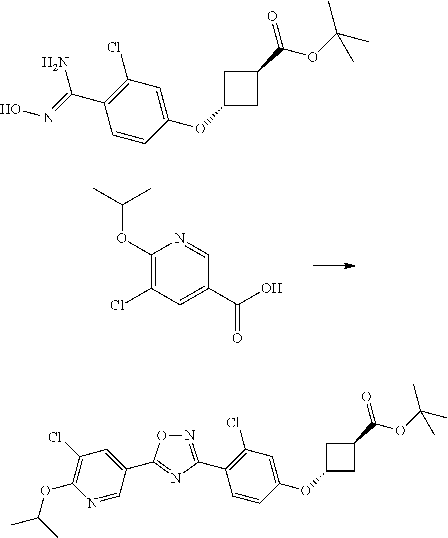

A solution of 5-chloro-6-isopropoxynicotinic acid (3.42 g, 15.87 mmol) and 1H-benzo[d][1,2,3]triazol-1-ol hydrate (2.430 g, 15.87 mmol) in CH₂Cl₂ (100 mL) was treated with EDC (3.04 g, 15.87 mmol) and the reaction was stirred at RT for 2 h. The mixture was washed twice with water (50 mL), dried over sodium sulfate, filtered and concentrated. The residue was dissolved in NMP (100 mL) and (1r,3r)-tert-butyl 3-(3-chloro-4-((Z)—N′-hydroxycarbamimidoyl)phenoxy)cyclobutanecarboxylate (5.23 g, 14.43 mmol) was added and the mixture was heated at about 110° C. for about 18 h. The reaction was cooled to room temperature, diluted with water (200 mL) and extracted with ethyl acetate (200 mL). The ethyl acetate layer was washed with saturated sodium bicarbonate solution (100 mL), saturated ammonium chloride solution (100 mL) and with saturated NaCl solution (100 mL). The organic layer was dried over sodium sulfate, filtered and concentrated. The residue was further purified on silica gel using a gradient 10% to 20% ethyl acetate/heptane. Product fractions were concentrated to about 100 mL and filtered to yield 3-{3-Chloro-4-[5-(5-chloro-6-isopropoxy-pyridin-3-yl)-[1,2,4]oxadiazol-3-yl]-phenoxy}-cyclobutanecarboxylic acid tert-butyl ester (6.52 g, 87%) as off white solid product. LC/MS (Table 1, Method c) Rₜ=3.30 min, m/z 520 (M+H)⁺; ¹H NMR (400 MHz, DMSO-d₆) δ ppm 8.91 (d, J=2.1, 1H), 8.53 (d, J=2.1, 1H), 7.95 (d, J=8.7, 1H), 7.12 (d, J=2.5, 1H), 7.03 (dd, J=8.8, 2.5, 1H), 5.50-5.40 (m, 1H), 5.02-4.90 (m, 1H), 3.13-3.06 (m, 1H), 2.73-2.63 (m, 2H), 2.43-2.32 (m, 2H), 1.45 (s, 9H), 1.39 (d, J=6.2, 6H).

### Example #94

### 3-{3-Chloro-4-[5-(5-chloro-6-isopropoxy-pyridin-3-yl)-[1,2,4]oxadiazol-3-yl]-phenoxy}-cyclobutanecarboxylic acid

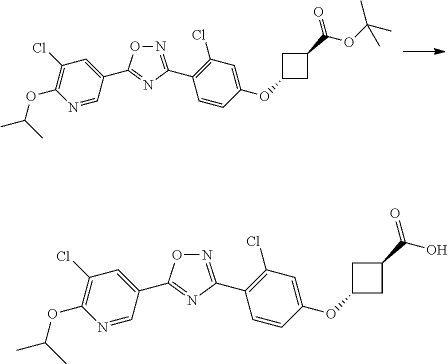

A solution of (1r,3r)-tert-butyl 3-(3-chloro-4-(5-(5-chloro-6-isopropoxypyridin-3-yl)-1,2,4-oxadiazol-3-yl)phenoxy)cyclobutanecarboxylate (6.42 g, 12.34 mmol) in DCM (100.0 mL) was treated with triisopropylsilane (2.53 mL, 12.34 mmol) and then TFA (100 mL) at RT for about 45 min. The mixture was concentrated to yield an oil and purified on silica gel using a gradient from 0-10% MeOH. Product fractions were combined and concentrated. The residue was triturated with 1:1/MeOH water (50 mL), filtered, washed with water and dried under vacuum to yield 3-{3-Chloro-4-[5-(5-chloro-6-isopropoxy-pyridin-3-yl)-[1,2,4]oxadiazol-3-yl]-phenoxy}-cyclobutanecarboxylic acid (5.26 g, 92%) as white solid.

LC/MS (Table 1, Method c) Rₜ=2.70 min, m/z 464 (M+H)⁺; ¹H NMR (400 MHz, DMSO-d₆) δ ppm 12.36 (s, 1H), 8.88 (d, J=2.1, 1H), 8.50 (d, J=2.1, 1H), 7.94 (d, J=8.7, 1H), 7.11 (d, J=2.5, 1H), 7.03 (dd, J=8.8, 2.5, 1H), 5.50-5.38 (m, 1H), 5.03-4.90 (m, 1H), 3.12 (m, 1H), 2.71 (m, 2H), 2.43-2.32 (m, 2H), 1.39 (d, J=6.2, 6H).

### Preparation #90

### (1R,3S)-ethyl 3-(4-((Z)—N′-hydroxycarbamimidoyl)phenylamino)cyclopentane carboxylate

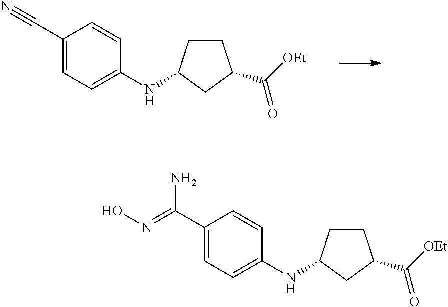

Into a round bottom flask was added (1R,3S)-ethyl 3-(4-cyanophenylamino)cyclopentane carboxylate (0.5 g, 1.936 mmol), hydroxylamine (50% by weight in water, 0.457 mL, 7.74 mmol) and ethanol (6 mL). The mixture was heated at about 60° C. for about 16 h. Upon completion of the reaction, the mixture was concentrated to dryness under reduced pressure to give (1R,3S)-ethyl 3-(4-((Z)—N′-hydroxycarbamimidoyl)phenylamino)cyclopentane carboxylate (0.506 g, 1.737 mmol, 90%) as an oil. LC/MS (Table 1, Method b) Rₜ=1.62 min, m/z 292 (M+H)+; ¹H NMR (400 MHz, DMSO-d₆) δ ppm 9.18 (s, 1H), 7.39-7.37 (d, 2H), 6.53-6.50 (d, 2H), 5.84-5.83 (d, 1H), 5.52 (s, 2H), 4.08-4.03 (q, 2H), 3.79-3.71 (m, 1H), 2.86-277 (m, 1H), 2.35-2.28 (m, 1H), 2.01-1.93 (m, 1H), 1.89-1.84 (m, 2H), 1.64-1.56 (m, 1H), 1.54-1.47 (m, 1H), 1.18-1.15 (t, 3H).

### Preparation #91

### (1R,3S)-ethyl 3-(4-(5-(5-chloro-6-isopropoxypyridin-3-yl)-1,2,4-oxadiazol-3-yl)phenylamino)cyclopentane carboxylate

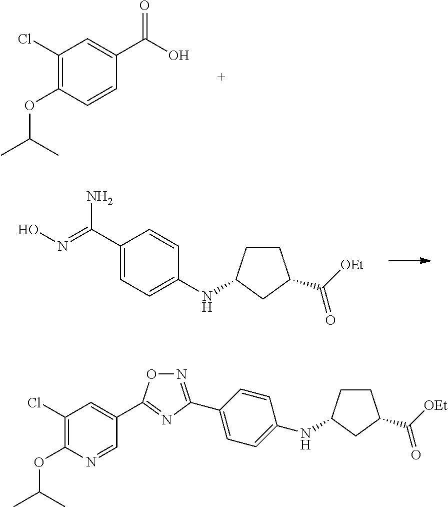

To a solution of (1R,3S)-Ethyl 3-(4-((Z)—N′-hydroxycarbamimidoyl)phenylamino)cyclopentane carboxylate (0.506 g, 1.737 mmol), EDC (0.366 g, 1.910 mmol), DIEA (0.334 mL, 1.910 mmol) and HOBT (0.293 g, 1.910 mmol) in DMF (10 mL) was added solution 5-chloro-6-isopropoxynicotinic acid (0.412 g, 1.910 mmol) and the reaction was stirred at RT under nitrogen for about 1 h and then heated at about 100° C. for about 16 h. The reaction was cooled and concentrated under reduced pressure and the residue was taken up in EtOAc (25 mL) and sequentially washed with a saturated NH₄Cl solution (20 mL), a saturated Na₂CO₃ solution (20 mL) and water (20 mL). The organic layer was dried over Na₂SO₄, filtered and concentrated under reduced pressure. The residue was purified on silica gel using a gradient of 15-50% EtOAc in Heptane to afford (1R,3S)-ethyl 3-(4-(5-(5-chloro-6-isopropoxypyridin-3-yl)-1,2,4-oxadiazol-3-yl)phenylamino)cyclopentane carboxylate (0.170 g, 0.361 mmol, 20.8%) as a pale yellow solid. LC/MS (Table 1, Method b) Rₜ=3.21 min, m/z 471 (M+H)⁺; ¹H NMR (400 MHz, DMSO-d₆) δ ppm 8.88 (d, 1H), 8.50 (d, 1H), 7.80-7.78 (d, 2H), 6.71-6.69 (d, 2H), 6.43-6.42 (d, 1H), 5.48-5.41 (m, 1H), 4.09-4.04 (q, 2H), 3.87-3.78 (m, 1H), 2.89-2.81 (m, 1H), 2.39-2.32 (m, 1H), 2.06-1.97 (m, 1H), 1.92-1.87 (m, 2H), 1.70-1.62 (m, 1H), 1.57-1.50 (m, 1H), 1.39-1.38 (d, 6H), 1.19-1.15 (t, 3H).

### Example #95

### (1R,3S)-3-(4-(5-(5-chloro-6-isopropoxypyridin-3-yl)-1,2,4-oxadiazol-3-yl)phenylamino)cyclopentane carboxylic acid

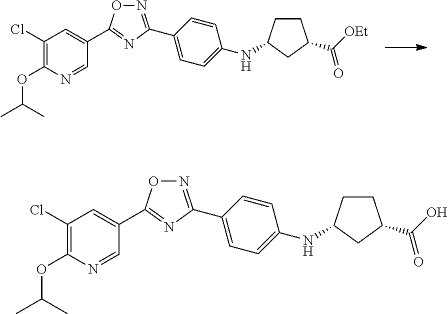

To a solution of (1R,3S)-ethyl 3-(4-(5-(5-chloro-6-isopropoxypyridin-3-yl)-1,2,4-oxadiazol-3-yl)phenylamino)cyclopentane carboxylate (170 mg, 0.361 mmol) in dioxane (3.6 mL) was added 2 M potassium hydroxide (0.722 mL, 1.444 mmol) and the mixture was stirred at RT for about 16 h. The reaction was neutralized by addition of 2 M HCl (750 μL) and the mixture is concentrated. The residue was triturated with water and aqueous ammonium acetate solution. The resulting solid was filtered and washed with water and dried to yield (1R,3S)-3-(4-(5-(5-chloro-6-isopropoxypyridin-3-yl)-1,2,4-oxadiazol-3-yl)phenylamino)cyclopentane carboxylic acid (141.6 mg, 0.317 mmol, 88%) as a tan solid. LC/MS (Table 1, Method b) Rt=2.81 min, m/z 433 (M+H)+; 1H NMR (400 MHz, DMSO-d6) δ ppm 8.88-87 (d, 1H), 8.50-8.49 (d, 1H), 7.81-7.79 (d, 2H), 6.74-6.71 (d, 2H), 5.49-5.40 (m, 1H), 3.85-3.78 (m, 1H), 2.82-2.73 (m, 1H), 2.37-2.30 (m, 1H), 2.05-1.96 (m, 1H), 1.91-1.85 (m, 2H), 1.69-1.62 (m, 1H), 1.58-1.50 (m, 1H), 1.39-1.38 (d, 6H).

### Preparation #I-1: Isomerization

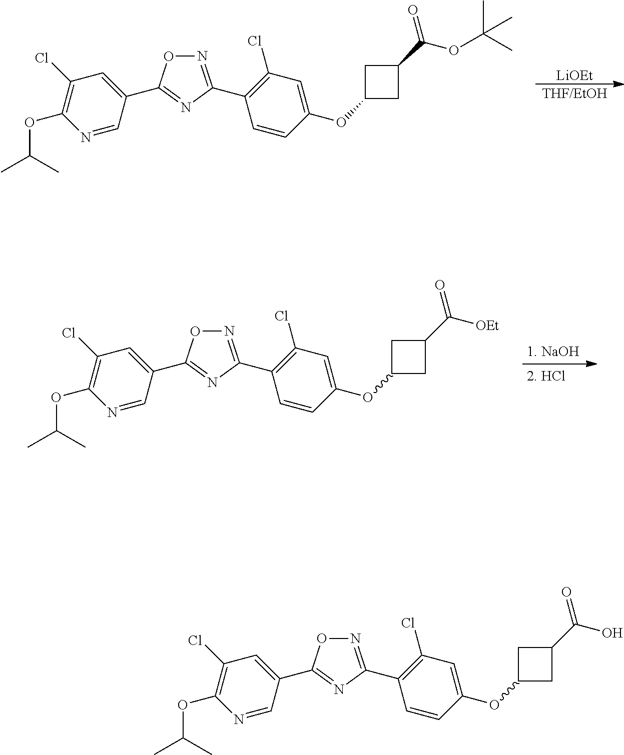

n-BuLi (about 1 equivalent) is added to about 20 mL THF at 0° C., followed by ethanol and trans-tert-butyl 3-(4-(5-(5-chloro-6-isopropoxypyridin-3-yl)-1,2,4-oxadiazol-3-yl)phenoxy)cyclobutanecarboxylate. Warmed to about room temperature over about 5 h then stirred about 18 h. NaOH (4 eq, 5% in water) is added. The reaction is stirred for about 4 h at rt and the pH is adjusted to about 2 with about 12 N HCl. The precipitate is filtered, washed with water and 1:1 ethanol water then dried. Yield 1.32 g. 82:14 cis/trans products identical to those described above (74% yield).

What is claimed is:

1. A compound of Formula (I) 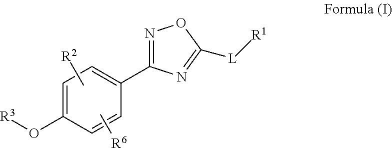
pharmaceutically acceptable salts, biologically active metabolites, solvates, hydrates, prodrugs, racemates, enantiomers or stereoisomers thereof, wherein
L is a bond or optionally substituted (C₁-C₃)alkyl;
R¹ is —C(O)—NH-phenyl, —NH—C(O)-furanyl, —NH—S(O)₂-optionally substituted phenyl, —O-optionally substituted (C₁-C₃)alkyl, —S-optionally substituted (C₁-C₃)alkyl, optionally substituted (C₂-C₆)alkyl, optionally substituted amino, optionally substituted (C₃-C₆)cycloalkyl, —(CH₂)(C₃)alkyl, optionally substituted tetrahydrobenzofuranyl, optionally substituted furanyl, optionally substituted tetrahydrofuranyl, optionally substituted 2,3-dihydroisoindolyl, optionally substituted isoindolinyl, optionally substituted imidazolyl, optionally substituted 5,6-dihydro imidazo[1,2-a]pyrazinyl, optionally substituted imidazo[1,2-a]pyrazinyl, optionally substituted indolyl, optionally substituted isoxazolyl, optionally substituted morpholinyl, optionally substituted naphthyl, optionally substituted phenyl, —O—CH₂-optionally substituted phenyl, —O— optionally substituted phenyl, —O-optionally substituted phenyl, optionally substituted piperidinyl, optionally substituted pyrazolyl, optionally substituted pyridinyl, optionally substituted pyrimidinyl, optionally substituted pyrrolidinyl, optionally substituted 1,2,3,4-tetrahydroisoquinolinyl, optionally substituted quinolinyl, optionally substituted 3,4-dihydroquinolinyl, optionally substituted 3,4-dihydroisoquinolinyl, optionally substituted 5,6,7,8-tetrahydroimidazo[1,2-a]pyrazinyl, optionally substituted pyrrolyl, optionally substituted pyrrolo[2,3-b]pyridinyl, optionally substituted quinolinyl, optionally substituted thiazolyl or optionally substituted thienyl;
R² is Br, Cl, CF₃, CN, or —O—(C₁-C₂)alkyl;
R³ is optionally substituted-(C₃-C₈)alkyl, deuterated —(C₂-C₆)alkyl, (C₄-C₅)alkenyl, (C₄-C₅)alkynyl, optionally substituted-(C₃-C₆)cycloalkyl, -optionally substituted (C₂-C₃)alkyl-O-optionally substituted (C₁-C₃)alkyl, -optionally substituted (C₁-C₃)alkyl-imidazolyl, -optionally substituted (C₁-C₃)alkyl-morpholinyl, -optionally substituted (C₁-C₃)alkyl-optionally substituted phenyl, -optionally substituted (C₁-C₃)alkyl-optionally substituted piperazinyl, -optionally substituted (C₁-C₃)alkyl-pyrrolidinyl, -optionally substituted (C₁-C₃)alkyl-piperidinyl, optionally substituted (C₁-C₃)alkyl-thienyl, tetrahydrofuranyl or optionally substituted thiazolyl; and
R⁶ is H;
provided that
R¹ is not substituted by optionally substituted cyclohexyl, —C(O)-cyclohexyl or —NH— cyclohexyl;
when L is (C₁-C₃)alkyl, R¹ is not optionally substituted isoxazolyl;
when R³ is optionally substituted (C₁)alkyl, L-R¹ is not cyclohexyl or —CH₂-cyclohexyl; and
provided that the compound is not

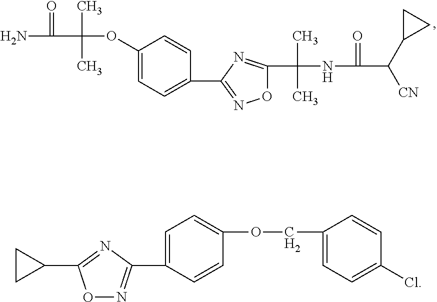

2. The compound of claim 1 wherein R¹ is optionally substituted by one or more substituents independently selected from Br, Cl, F, CF₃, CN, oxo, —C(═O)H, —N(R⁹)₂, optionally substituted (C₁-C₆)alkyl, optionally substituted (C₂-C₆)alkenyl, optionally substituted (C₂-C₆)alkynyl, optionally substituted azabicyclo[2.2.1]heptanyl, optionally substituted (C₃-C₆)cycloalkyl, —C(R⁹)₂-optionally substituted (C₃-C₆)cycloalkyl, —C(R⁹)₂-optionally substituted azetidinyl, —CR⁹₂-optionally substituted piperidinyl, —C(R⁹)₂-optionally substituted pyrrolidinyl, —C(R⁹)₂—N(R⁹)₂, —C(O)-optionally substituted (C₁-C₆)alkyl, —C(O)—NR⁹—(C₁-C₆)alkyl, —C(O)—O-optionally substituted (C₁-C₆)alkyl, —C(R⁹)₂—C(O)—O-optionally substituted (C₁-C₆)alkyl, —NR⁹-optionally substituted (C₃-C₆)cycloalkyl, —NR⁹-optionally substituted azetidinyl, —NR₉-furanyl, —NR⁹-optionally substituted pyrrolidinyl, —NR⁹—C(O)—O-optionally substituted (C₁-C₃)alkyl, —NR⁹-optionally substituted (C₁-C₆)alkyl, —NR⁹-optionally substituted (C₃-C₆)cycloalkyl, —NR⁹—C(O)-azetidinyl, —NR⁹—C(O)-furanyl, —NR⁹—C(O)-pyridinyl, —NR⁹—C(O)-optionally substituted pyrrolidinyl, —NR⁹—S(O)₂-optionally substituted phenyl, —O-optionally substituted (C₁-C₆)alkyl, —O-deuterated —(C₂-C₆)alkyl, —O-optionally substituted (C₂-C₆)alkenyl, —O-optionally substituted (C₃-C₆)cycloalkyl, —O-1H-benzo[d][1,2,3]triazolyl, —S(O)₂—N(R⁹)₂, —S(O)₂—NR⁹-optionally substituted (C₁-C₄)alkyl, optionally substituted azetidinyl, optionally substituted piperidinyl, optionally substituted pyridinyl, optionally substituted pyrrolidinyl, optionally substituted 1,2,4 oxadiazolyl, optionally substituted pyrrolidinyl, optionally substituted tetrazolyl, 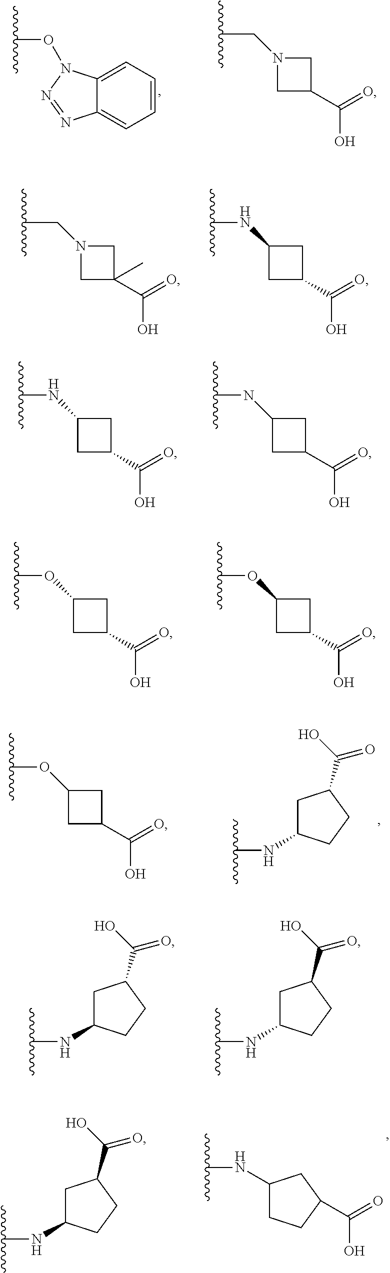 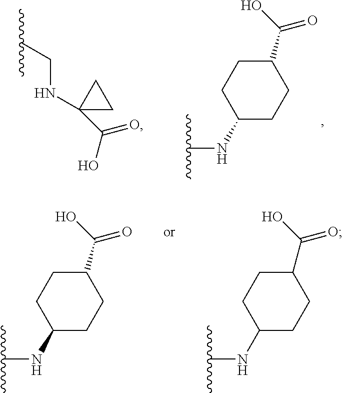
and
wherein each R⁹ is independently selected from H or optionally substituted (C₁-C₆)alkyl.

3. The compound of claim 1 wherein the compound is a compound of Formula (Ia) 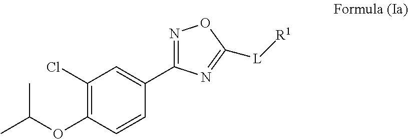
wherein L is a bond.

4. The compound of claim 3 wherein R¹ is optionally substituted tetrahydrobenzofuranyl, optionally substituted furanyl, optionally substituted 2,3-dihydroisoindolyl, optionally substituted isoindolinyl, optionally substituted imidazolyl, optionally substituted 5,6-dihydro imidazo[1,2-a]pyrazinyl, optionally substituted imidazo[1,2-a]pyrazinyl, optionally substituted indolyl, optionally substituted isoxazolyl, optionally substituted pyrazolyl, optionally substituted pyridinyl, optionally substituted pyrimidinyl, optionally substituted pyrrolidinyl, optionally substituted 1,2,3,4-tetrahydroisoquinolinyl, optionally substituted quinolinyl, optionally substituted 3,4-dihydroquinolinyl, optionally substituted 3,4-dihydroisoquinolinyl, optionally substituted 5,6,7,8-tetrahydroimidazo[1,2-a]pyrazinyl, optionally substituted pyrrolyl, optionally substituted pyrrolo[2,3-b]pyridinyl, optionally substituted quinolinyl, optionally substituted thiazolyl or optionally substituted thienyl.

5. The compound of claim 4 wherein R¹ is optionally substituted furanyl, optionally substituted imidazolyl, optionally substituted isoxazolyl, optionally substituted pyrazolyl, optionally substituted pyridinyl, optionally substituted pyrimidinyl, optionally substituted pyrrolidinyl, optionally substituted pyrrolyl, optionally substituted thiazolyl or optionally substituted thienyl.

6. The compound of claim 1 wherein R¹ is optionally substituted phenyl or optionally substituted indolyl.

7. The compound of claim 1 wherein
L is optionally substituted (C₁-C₃)alkyl;
R¹ is —C(O)—NH-phenyl, —NH—C(O)-furanyl, —NH—S(O)₂-optionally substituted phenyl, optionally substituted —O—(C₁-C₃)alkyl, —S—(C₁-C₃)alkyl, optionally substituted benzyloxy, optionally substituted (C₃-C₆)cycloalkyl, optionally substituted imidazolyl, morpholinyl, optionally substituted naphthyl, optionally substituted phenyl, optionally substituted phenoxy, optionally substituted piperazinyl, optionally substituted piperidinyl, optionally substituted pyridinyl, optionally substituted pyrrolidinyl or optionally substituted thienyl;
R² is Cl;
R³ is isopropyl; and
R⁶ is H.

8. The compound of claim 1 wherein L is CH₂ and R¹ is optionally substituted phenyl or optionally substituted (C₃-C₆)cycloalkyl.

9. The compound of claim 1 wherein the compound is
4-[3-(3-chloro-4-isopropoxy-phenyl)-[1,2,4]oxadiazol-5-yl]-3-methyl-pyridine;
3-[3-chloro-4-(1-ethyl-propoxy)-phenyl]-5-o-tolyl-[1,2,4]oxadiazole;
3-(3-chloro-4-isopropoxyphenyl)-5-(3-chloropyridin-4-yl)-[1,2,4]-oxadiazole;
3-chloro-4-(3-(3-chloro-4-isopropoxyphenyl)-1,2,4-oxadiazol-5-yl)benzonitrile;
1-(3-chloro-4-(3-(3-chloro-4-isopropoxyphenyl)-1,2,4-oxadiazol-5-yl)benzyl)-3-methylazetidine-3-carboxylic acid;
tert-butyl 3-(4-(3-(3-chloro-4-isopropoxyphenyl)-1,2,4-oxadiazol-5-yl)-1H-indol-1-yl)propanoate;
tert-butyl 4-(4-(3-(3-chloro-4-isopropoxyphenyl)-1,2,4-oxadiazol-5-yl)-1H-indol-1-yl)butanoate;
3-(4-(3-(3-chloro-4-isopropoxyphenyl)-1,2,4-oxadiazol-5-yl)-1H-indol-1-yl)propanoic acid;
(1R,3S)-3-(4-(3-(3-chloro-4-isopropoxyphenyl)-1,2,4-oxadiazol-5-yl)phenylamino)cyclopentanecarboxylic acid;
3-(4-(3-(3-chloro-4-isopropoxyphenyl)-1,2,4-oxadiazol-5-yl)phenoxy)propane-1,2-diol;
(R)-3-{3-chloro-4-[3-(3-chloro-4-isopropoxy-phenyl)-[1,2,4]oxadiazol-5-yl]-phenoxy}-propane-1,2-diol;
3-{3-chloro-4-[5-(5-chloro-6-isopropoxy-pyridin-3-yl)-[1,2,4]oxadiazol-3-yl]-phenoxy}-cyclobutanecarboxylic acid;
3-(4-(3-(3-chloro-4-isopropoxyphenyl)-1,2,4-oxadiazol-5-yl)phenylamino)propylphosphonic acid;
ethyl 3-(4-(3-(3-chloro-4-isopropoxyphenyl)-1,2,4-oxadiazol-5-yl)benzylidene)cyclobutanecarboxylate;
ethyl 3-(4-(3-(3-chloro-4-isopropoxyphenyl)-1,2,4-oxadiazol-5-yl)benzyl)cyclobutanecarboxylate;
3-(4-(3-(3-chloro-4-isopropoxyphenyl)-1,2,4-oxadiazol-5-yl)benzyl)cyclobutanecarboxylic acid;
5-(3-(3-chloro-4-isopropoxyphenyl)-1,2,4-oxadiazol-5-yl)-1-methyl-1H-pyrazol-3-amine;
3-(3-chloro-4-isopropoxyphenyl)-5-(1H-indol-5-yl)-1,2,4-oxadiazole;
1-(4-(3-(3-chloro-4-isopropoxyphenyl)-1,2,4-oxadiazol-5-yl)benzyl)pyrrolidine-3-carboxylic acid;
3-amino-1-(4-(3-(3-chloro-4-isopropoxyphenyl)-1,2,4-oxadiazol-5-yl)benzyl)pyrrolidine-3-carboxylic acid;
(S)-1-(4-(3-(3-chloro-4-isopropoxyphenyl)-1,2,4-oxadiazol-5-yl)benzyl)pyrrolidine-3-carboxylic acid;
(R)-1-(4-(3-(3-chloro-4-isopropoxyphenyl)-1,2,4-oxadiazol-5-yl)benzyl)pyrrolidine-3-carboxylic acid;
(S)-1-(4-(3-(3-chloro-4-isopropoxyphenyl)-1,2,4-oxadiazol-5-yl)benzyl)azetidine-2-carboxylic acid;
4-(4-(3-(3-chloro-4-isopropoxyphenyl)-1,2,4-oxadiazol-5-yl)-1H-indol-1-yl)butanoic acid;
3-(4-(3-(3-chloro-4-isopropoxyphenyl)-1,2,4-oxadiazol-5-yl)-1H-indol-1-yl)-2-fluoropropanoic acid;
3-(4-(3-(3-chloro-4-isopropoxyphenyl)-1,2,4-oxadiazol-5-yl)-1H-indol-1-yl)-2-methylpropanoic acid;
3-(4-(3-(3-chloro-4-isopropoxyphenyl)-1,2,4-oxadiazol-5-yl)-1H-indol-1-yl)-2,2-dimethylpropanoic acid;
3-(4-(3-(3-chloro-4-isopropoxyphenyl)-1,2,4-oxadiazol-5-yl)-1H-pyrrolo[2,3-b]pyridin-1-yl)propanoic acid;
(1R,3S)-3-{4-[3-(5-chloro-6-isopropoxy-pyridin-3-yl)-[1,2,4]oxadiazol-5-yl]-3-methyl-phenylamino}-cyclopentane carboxylic acid;
4-(4-(3-(3-chloro-4-isopropoxyphenyl)-1,2,4-oxadiazol-5-yl)phenylamino)-3,3-dimethylbutanoic acid;
4-(4-(3-(3-chloro-4-isopropoxyphenyl)-1,2,4-oxadiazol-5-yl)phenylamino)butanoic acid;
1-(4-(3-(3-chloro-4-isopropoxyphenyl)-1,2,4-oxadiazol-5-yl)phenyl)pyrrolidine-3-carboxylic acid;
2-(1-(4-(3-(3-chloro-4-isopropoxyphenyl)-1,2,4-oxadiazol-5-yl)phenyl)pyrrolidin-3-yl)acetic acid;
(1R,3S)-3-(4-(3-(3-bromo-4-isopropoxyphenyl)-1,2,4-oxadiazol-5-yl)phenylamino)cyclopentanecarboxylic acid;
(1R,3S)-3-(4-(3-(4-isopropoxy-3-(trifluoromethyl)phenyl)-1,2,4-oxadiazol-5-yl)phenylamino)cyclopentanecarboxylic acid;
(1R,3S)-3-(4-(3-(5-chloro-6-isopropoxypyridin-3-yl)-1,2,4-oxadiazol-5-yl)phenylamino)cyclopentanecarboxylic acid;
(R)-1-(4-(3-(3-chloro-4-isopropoxyphenyl)-1,2,4-oxadiazol-5-yl)phenyl)pyrrolidin-3-amine, acetic acid;
(1R,2S)-2-(4-(3-(3-chloro-4-isopropoxyphenyl)-1,2,4-oxadiazol-5-yl)phenylamino)cyclopentanecarboxylic acid;
(1S,2R)-2-(4-(3-(3-chloro-4-isopropoxyphenyl)-1,2,4-oxadiazol-5-yl)phenylamino)cyclohexanecarboxylic acid;
(S)-1-(3-(4-(3-(3-chloro-4-isopropoxyphenyl)-1,2,4-oxadiazol-5-yl)phenylamino)pyrrolidin-1-yl)ethanone;
(1R,2R)-2-(4-(3-(3-chloro-4-isopropoxyphenyl)-1,2,4-oxadiazol-5-yl)phenylamino)cyclohexanecarboxylic acid;
(1R,2S)-2-(4-(3-(3-chloro-4-isopropoxyphenyl)-1,2,4-oxadiazol-5-yl)phenylamino)cyclohexanecarboxylic acid;
(1R,2S)-2-(4-(3-(3-chloro-4-isopropoxyphenyl)-1,2,4-oxadiazol-5-yl)phenylamino)cyclohexanecarboxylic acid;
(1S,2R)-2-(4-(3-(3-chloro-4-isopropoxyphenyl)-1,2,4-oxadiazol-5-yl)phenylamino)cyclohexanecarboxylic acid;
(1R,2R)-2-(4-(3-(3-chloro-4-isopropoxyphenyl)-1,2,4-oxadiazol-5-yl)phenylamino)cyclohexanecarboxylic acid;
(1S,2S)-2-(4-(3-(3-chloro-4-isopropoxyphenyl)-1,2,4-oxadiazol-5-yl)phenylamino)cyclohexanecarboxylic acid;
(1S,2R)-2-(4-(3-(3-chloro-4-isopropoxyphenyl)-1,2,4-oxadiazol-5-yl)phenylamino)cyclopentanecarboxylic acid;
(1S,2S)-2-(4-(3-(3-chloro-4-isopropoxyphenyl)-1,2,4-oxadiazol-5-yl)phenylamino)cyclohexanecarboxylic acid;
(S)—N-(4-(3-(3-chloro-4-isopropoxyphenyl)-1,2,4-oxadiazol-5-yl)phenyl)-1-(methylsulfonyl)pyrrolidin-3-amine;
(S)-2-(3-(4-(3-(3-chloro-4-isopropoxyphenyl)-1,2,4-oxadiazol-5-yl)phenylamino)pyrrolidin-1-yl)acetic acid;
(1R,3S)-3-(2-bromo-4-(3-(3-chloro-4-isopropoxyphenyl)-1,2,4-oxadiazol-5-yl)phenylamino)cyclopentanecarboxylic acid;
(1R,3S)-3-(2-bromo-3-(3-(3-chloro-4-isopropoxyphenyl)-1,2,4-oxadiazol-5-yl)phenylamino)cyclopentanecarboxylic acid;
(1R,3S)-3-(5-(3-(3-chloro-4-isopropoxyphenyl)-1,2,4-oxadiazol-5-yl)pyridin-2-ylamino)cyclopentanecarboxylic acid;
(1R,3S)-3-(4-(3-(3-chloro-4-isopropoxyphenyl)-1,2,4-oxadiazol-5-yl)pyridin-2-ylamino)cyclopentanecarboxylic acid;
(3S)-3-(4-(3-(3-chloro-4-isopropoxyphenyl)-1,2,4-oxadiazol-5-yl)phenylamino)-1-methylcyclopentanecarboxylic acid;
2-((1R,3S)-3-(4-(3-(3-chloro-4-isopropoxyphenyl)-1,2,4-oxadiazol-5-yl)phenylamino)cyclopentyl)acetic acid;
(1S,3S)-3-(4-(3-(3-chloro-4-isopropoxyphenyl)-1,2,4-oxadiazol-5-yl)phenylamino)-1-methylcyclopentanecarboxylic acid;
(1R,3S)-3-(4-(3-(3-chloro-4-isopropoxyphenyl)-1,2,4-oxadiazol-5-yl)phenylamino)-1-methylcyclopentanecarboxylic acid;
(3S)-3-(4-(3-(3-chloro-4-isopropoxyphenyl)-1,2,4-oxadiazol-5-yl)phenylamino)-1-fluorocyclopentanecarboxylic acid;
(1R,3S)-3-(4-(5-(3-chloro-4-isopropoxyphenyl)-1,2,4-oxadiazol-3-yl)phenylamino)cyclopentanecarboxylic acid;
(3S)-3-(4-(3-(3-chloro-4-isopropoxyphenyl)-1,2,4-oxadiazol-5-yl)phenylamino)-1-hydroxycyclopentanecarboxylic acid;
(1R,3S)-3-(3-chloro-4-(3-(3-chloro-4-isopropoxyphenyl)-1,2,4-oxadiazol-5-yl)phenylamino)cyclopentanecarboxylic acid;
(1R,3S)-3-(4-(3-(3-bromo-4-isopropoxyphenyl)-1,2,4-oxadiazol-5-yl)-3-chlorophenylamino)cyclopentanecarboxylic acid;
(1R,3S)-3-(3-bromo-4-(3-(3-chloro-4-isopropoxyphenyl)-1,2,4-oxadiazol-5-yl)phenylamino)cyclopentanecarboxylic acid;
(1R,3S)-3-(4-(3-(3-chloro-4-isopropoxyphenyl)-1,2,4-oxadiazol-5-yl)-2-(trifluoromethyl)phenylamino)cyclopentanecarboxylic acid;
(R)-3-(4-(3-(3-chloro-4-isopropoxyphenyl)-1,2,4-oxadiazol-5-yl)-1H-indol-1-yl)-2-methylpropanoic acid;
(S)-3-(4-(3-(3-chloro-4-isopropoxyphenyl)-1,2,4-oxadiazol-5-yl)-1H-indol-1-yl)-2-methylpropanoic acid;
(1R,2S)-methyl 2-(4-(3-(3-chloro-4-isopropoxyphenyl)-1,2,4-oxadiazol-5-yl)phenylamino)cyclopentanecarboxylate;
(1S,2R)-methyl 2-(4-(3-(3-chloro-4-isopropoxyphenyl)-1,2,4-oxadiazol-5-yl)phenylamino)cyclopentanecarboxylate;
(1R,2R)-2-(4-(3-(3-chloro-4-isopropoxyphenyl)-1,2,4-oxadiazol-5-yl)phenylamino)cyclopentanecarboxylic acid;
(1S,2S)-2-(4-(3-(3-chloro-4-isopropoxyphenyl)-1,2,4-oxadiazol-5-yl)phenylamino)cyclopentanecarboxylic acid;
3-(3-chloro-4-isopropoxyphenyl)-5-(1-methyl-1,2,3,4-tetrahydroquinolin-6-yl)-1,2,4-oxadiazole;
(R)-3-(4-(3-(4-(tetrahydrofuran-3-yloxy)-3-(trifluoromethyl)phenyl)-1,2,4-oxadiazol-5-yl)-1H-indol-1-yl)propanoic acid;
(1R,3S)-3-[4-(5-biphenyl-2-yl-[1,2,4]oxadiazol-3-yl)-2-methyl-phenylamino]-cyclopentanecarboxylic acid;
(1R,3S)-3-[4-(5-biphenyl-3-yl-[1,2,4]oxadiazol-3-yl)-2-methyl-phenylamino]-cyclopentanecarboxylic acid;
(1R,3S)-3-[4-(5-biphenyl-4-yl-[1,2,4]oxadiazol-3-yl)-2-methyl-phenylamino]-cyclopentanecarboxylic acid;
(1R,3S)-3-{4-[5-(4-cyclohexyl-phenyl)-[1,2,4]oxadiazol-3-yl]-2-methyl-phenylamino}-cyclopentanecarboxylic acid;
(1R,3S)-3-((4-(3-(3-chloro-4-isopropoxyphenyl)-1,2,4-oxadiazol-5-yl)phenyl)(methyl)amino)cyclopentanecarboxylic acid;
methyl 3-(5-(3-(3-chloro-4-isopropoxyphenyl)-1,2,4-oxadiazol-5-yl)-1H-indol-1-yl)cyclopentanecarboxylate;
3-(5-(3-(3-chloro-4-isopropoxyphenyl)-1,2,4-oxadiazol-5-yl)-1H-indol-1-yl)cyclopentanecarboxylic acid;
(1R,3S)-3-(4-(3-(3-chloro-4-isopropoxyphenyl)-1,2,4-oxadiazol-5-yl)-2-ethynylphenylamino)cyclopentanecarboxylic acid;
(1R,3S)-3-(5-(3-(3-chloro-4-isopropoxyphenyl)-1,2,4-oxadiazol-5-yl)-1H-indol-1-yl)cyclopentanecarboxylic acid;
(1S,4R)-2-(4-(3-(3-chloro-4-isopropoxyphenyl)-1,2,4-oxadiazol-5-yl)-3-methoxyphenyl)-2-azabicyclo[2.2.1]heptan-3-one;
(1R,3S)-3-(4-(3-(3-chloro-4-isopropoxyphenyl)-1,2,4-oxadiazol-5-yl)-3-methoxyphenylamino)cyclopentanecarboxylic acid;
(R)-3-(3-(3-(3-chloro-4-isopropoxyphenyl)-1,2,4-oxadiazol-5-yl)-2-methylphenoxy)propane-1,2-diol;
N-(4-(3-(3-chloro-4-isopropoxyphenyl)-1,2,4-oxadiazol-5-yl)phenyl)isonicotinamide;
N-(4-(3-(3-chloro-4-isopropoxyphenyl)-1,2,4-oxadiazol-5-yl)phenyl)isonicotinamide;
(3S)-3-(4-(3-(3-chloro-4-isopropoxyphenyl)-1,2,4-oxadiazol-5-yl)phenylamino)-1-hydroxycyclopentanecarboxylic acid;
(1R,3S)-3-(4-(5-(3-chloro-4-isopropoxyphenyl)-1,2,4-oxadiazol-3-yl)phenylamino)cyclopentanecarboxylic acid;
1-amino-3-(4-(3-(3-chloro-4-isopropoxyphenyl)-1,2,4-oxadiazol-5-yl)phenoxy)cyclopentanecarboxylic acid;
1-amino-3-(3-chloro-4-(5-(5-chloro-6-isopropoxypyridin-3-yl)-1,2,4-oxadiazol-3-yl)phenoxy)cyclopentanecarboxylic acid;
(1R,3S)-3-(4-(5-(3-chloro-4-isopropoxyphenyl)-1,2,4-oxadiazol-3-yl)-3-methylphenylamino)cyclopentanecarboxylic acid;
(1S,4R)-2-(4-(5-(5-chloro-6-isopropoxypyridin-3-yl)-1,2,4-oxadiazol-3-yl)-3-methylphenyl)-2-azabicyclo[2.2.1]heptan-3-one;
(1R,3S)-3-(4-(5-(5-chloro-6-isopropoxypyridin-3-yl)-1,2,4-oxadiazol-3-yl)-3-methylphenylamino)cyclopentanecarboxylic acid;
methyl 2-(5-(3-(3-(3-chloro-4-isopropoxyphenyl)-1,2,4-oxadiazol-5-yl)phenyl)-2H-tetrazol-2-yl)acetate;
3-(3-(3-(3-chloro-4-isopropoxyphenyl)-1,2,4-oxadiazol-5-yl)phenyl)-1,2,4-oxadiazol-5(2M-one;
(1R,3S)-3-(4-(3-(3-chloro-4-isopropoxyphenyl)-1,2,4-oxadiazol-5-yl)-3-ethylphenylamino)cyclopentanecarboxylic acid;
(1R,3S)-3-(4-(3-(3-chloro-4-isopropoxyphenyl)-1,2,4-oxadiazol-5-yl)-3-cyanophenylamino)cyclopentanecarboxylic acid;
(2R,4R)-4-(4-(3-(3-chloro-4-isopropoxyphenyl)-1,2,4-oxadiazol-5-yl)phenylamino)pyrrolidine-2-carboxylic acid;
2-(4-(3-(4-isopropoxy-3-(trifluoromethyl)phenyl)-1,2,4-oxadiazol-5-yl)phenylamino)propan-1-ol;
(R)-2-(4-(3-(4-isopropoxy-3-(trifluoromethyl)phenyl)-1,2,4-oxadiazol-5-yl)phenoxy)propanoic acid;
(R)—N-(2-hydroxyethyl)-2-(4-(3-(4-isopropoxy-3-(trifluoromethyl)phenyl)-1,2,4-oxadiazol-5-yl)phenoxy)propanamide;
(R)-2-(4-(3-(4-isopropoxy-3-(trifluoromethyl)phenyl)-1,2,4-oxadiazol-5-yl)phenoxy)propanal;
3-{3-Chloro-4-[5-(5-chloro-6-isopropoxy-pyridin-3-yl)-[1,2,4]oxadiazol-3-yl]-phenoxy}-cyclobutanecarboxylic acid; or
(1R,3S)-3-(4-(5-(5-chloro-6-isopropoxypyridin-3-yl)-1,2,4-oxadiazol-3-yl)phenylamino)cyclopentane carboxylic acid.

10. A compound of Formula (II) 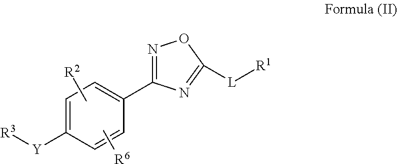
pharmaceutically acceptable salts, biologically active metabolites, solvates, hydrates, prodrugs, enantiomers or stereoisomers thereof, wherein
Y is a bond;
L is a bond or CH₂;
R¹ is optionally substituted (C₁-C₄)alkyl, optionally substituted indolyl or optionally substituted phenyl;
R² is CF₃,
R³ is H, morpholinyl, optionally substituted piperidine or (C₃-C₅)cycloalkyl; and
R⁶ is H.

11. The compound of claim 10 wherein R¹ is optionally substituted by one or more substituents independently selected from Cl, F, CN, optionally substituted (C₁-C₃)alkyl, —CH₂-optionally substituted azetidinyl, —CH₂-optionally substituted pyrrolidinyl, —CH₂NR^{c}R^{d}, —NH-optionally substituted (C₃-C₆)cycloalkyl, optionally substituted piperidinyl, 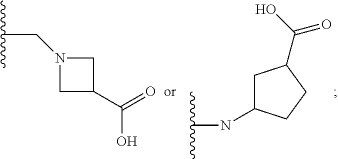
wherein R^{c} and R^{d} are independently H, optionally substituted (C₁-C₆)alkyl or optionally substituted (C₃-C₆)cycloalkyl.

12. The compound of claim 11 wherein the compound is
1-((4-(3-(4-(4-fluoropiperidin-1-yl)-3-(trifluoromethyl)phenyl)-1,2,4-oxadiazol-5-yl)benzylamino)methyl)cyclopropanecarboxylic acid;
(R)-1-(4-(3-(4-(4-fluoropiperidin-1-yl)-3-(trifluoromethyl)phenyl)-1,2,4-oxadiazol-5-yl)benzyl)pyrrolidine-3-carboxylic acid;
(S)-1-(4-(3-(4-(4-fluoropiperidin-1-yl)-3-(trifluoromethyl)phenyl)-1,2,4-oxadiazol-5-yl)benzyl)pyrrolidine-3-carboxylic acid;
1-(4-(3-(4-(4-fluoropiperidin-1-yl)-3-(trifluoromethyl)phenyl)-1,2,4-oxadiazol-5-yl)benzylamino)cyclopropanecarboxylic acid;
1-(4-{3-[4-(4-fluoro-piperidin-1-yl)-3-trifluoromethyl-phenyl]-[1,2,4]oxadiazol-5-yl}-benzylamino)-cyclopropanecarboxylic acid;
1-(4-{3-[4-(4-fluoro-piperidin-1-yl)-3-trifluoromethyl-phenyl]-[1,2,4]oxadiazol-5-yl}-benzyl)-pyrrolidine-3-carboxylic acid;
1-(4-{3-[4-(4-fluoro-piperidin-1-yl)-3-trifluoromethyl-phenyl]-[1,2,4]oxadiazol-5-yl}-benzyl)-4-methyl-pyrrolidine-3-carboxylic acid;
4-fluoro-piperidin-1-yl)-3-trifluoromethyl-phenyl]-[1,2,4]oxadiazol-5-yl}-benzylamino)-acetic acid;
[(S)-1-(4-{3-[4-(4-fluoro-piperidin-1-yl)-3-trifluoromethyl-phenyl]-[1,2,4]oxadiazol-5-yl}-benzyl)-pyrrolidin-2-yl]-acetic acid;
[1-(4-{3-[4-(4-fluoro-piperidin-1-yl)-3-trifluoromethyl-phenyl]-[1,2,4]oxadiazol-5-yl}-benzylamino)-cyclopropyl]-methanol;
1-(4-{3-[4-(4-fluoro-piperidin-1-yl)-3-trifluoromethyl-phenyl]-[1,2,4]oxadiazol-5-yl}-benzyl)-4,4-dimethyl-pyrrolidine-3-carboxylic acid;
1-[(4-{3-[4-(4-fluoro-piperidin-1-yl)-3-trifluoromethyl-phenyl]-[1,2,4]oxadiazol-5-yl}-benzylamino)-methyl]-cyclopropanol;
(1R,3S)-3-(4-(3-(3-chloro-4-isopropoxyphenyl)-1,2,4-oxadiazol-5-yl)-3-(trifluoromethyl)phenylamino)cyclopentanecarboxylic acid;
(1R,3S)-3-(4-(3-(3-chloro-4-isopropoxyphenyl)-1,2,4-oxadiazol-5-yl)-3-methylphenylamino)cyclopentanecarboxylic acid;
(1R,3S)-3-(4-(3-(3-chloro-4-isopropoxyphenyl)-1,2,4-oxadiazol-5-yl)-2-methylphenylamino)cyclopentanecarboxylic acid;
4-(4-(3-(3-chloro-4-isopropoxyphenyl)-1,2,4-oxadiazol-5-yl)phenylamino)-2-methylbutanoic acid;
tert-butyl 2-(4-(3-(3-chloro-4-isopropoxyphenyl)-1,2,4-oxadiazol-5-yl)phenylamino)acetate;
tert-butyl 3-(4-(3-(3-chloro-4-isopropoxyphenyl)-1,2,4-oxadiazol-5-yl)phenylamino)propanoate;
1-amino-3-(4-(3-(3-chloro-4-isopropoxyphenyl)-1,2,4-oxadiazol-5-yl)phenoxy)cyclopentanecarboxylic acid;
3-{4-[3-(3-chloro-4-isopropoxy-phenyl)-[1,2,4]oxadiazol-5-yl]-phenoxy}-cyclobutanecarboxylic acid;
2-(6-(3-(3-chloro-4-isopropoxyphenyl)-1,2,4-oxadiazol-5-yl)-3,4-dihydroquinolin-1(2H)-yl)acetic acid;
3-(6-(3-(3-chloro-4-isopropoxyphenyl)-1,2,4-oxadiazol-5-yl)-3,4-dihydroquinolin-1(2H)-yl)propanoic acid;
(E)-4-(3-(3-(3-chloro-4-isopropoxyphenyl)-1,2,4-oxadiazol-5-yl)-2-methylphenoxy)but-2-enoic acid;
4-(3-(3-(3-chloro-4-isopropoxyphenyl)-1,2,4-oxadiazol-5-yl)-2-methylphenoxy)butanoic acid;
4-(3-(3-(3-chloro-4-isopropoxyphenyl)-1,2,4-oxadiazol-5-yl)-4-methylphenoxy)butanoic acid;
(3-{4-[3-(3-chloro-4-isopropoxy-phenyl)-[1,2,4]oxadiazol-5-yl]-phenylamino}-propyl)-phosphonic acid diethyl ester;
(3-{4-[3-(3-chloro-4-isopropoxy-phenyl)-[1,2,4]oxadiazol-5-yl]-benzylamino}-propyl)-phosphonic acid;
(1S,3R)-3-(4-(3-(3-chloro-4-isopropoxyphenyl)-1,2,4-oxadiazol-5-yl)phenylamino)cyclopentylphosphonic acid;
(1R,3R)-3-(4-(3-(3-chloro-4-isopropoxyphenyl)-1,2,4-oxadiazol-5-yl)phenylamino)cyclopentylphosphonic acid;
(1R,3R)-3-(2-bromo-4-(3-(3-chloro-4-isopropoxyphenyl)-1,2,4-oxadiazol-5-yl)phenylamino)cyclopentylphosphonic acid;
(1R,3S)-3-(4-(3-(3-chloro-4-isopropoxyphenyl)-1,2,4-oxadiazol-5-yl)phenylamino)cyclopentylphosphonic acid;
(1R,3S)-3-(2-bromo-4-(3-(3-chloro-4-isopropoxyphenyl)-1,2,4-oxadiazol-5-yl)phenylamino)cyclopentylphosphonic acid;
(1S,3S)-3-(4-(3-(3-chloro-4-isopropoxyphenyl)-1,2,4-oxadiazol-5-yl)phenylamino)cyclopentylphosphonic acid;
3-(4-(3-(3-chloro-4-isopropoxyphenyl)-1,2,4-oxadiazol-5-yl)benzyl)cyclobutanecarboxylic acid;
3-(4-(3-(3-chloro-4-isopropoxyphenyl)-1,2,4-oxadiazol-5-yl)benzyl)cyclopentanecarboxylic acid;
1-(4-(3-(3-chloro-4-isopropoxyphenyl)-1,2,4-oxadiazol-5-yl)benzyl)azetidine-3-carboxylic acid;
2-(4-(3-(3-chloro-4-isopropoxyphenyl)-1,2,4-oxadiazol-5-yl)phenyl)propan-2-amine;
methyl 3-(2-(4-(3-(3-chloro-4-isopropoxyphenyl)-1,2,4-oxadiazol-5-yl)phenyl)propan-2-ylamino)propanoate;
3-(2-(4-(3-(3-chloro-4-isopropoxyphenyl)-1,2,4-oxadiazol-5-yl)phenyl)propan-2-ylamino)propanoic acid;
3-(3-chloro-4-isopropoxyphenyl)-5-(1H-indol-4-yl)-1,2,4-oxadiazole;
(4-(3-(3-chloro-4-isopropoxyphenyl)-1,2,4-oxadiazol-5-yl)phenyl)methanamine;
3-(3-(3-(3-chloro-4-isopropoxyphenyl)-1,2,4-oxadiazol-5-yl)cyclopentylamino)propanoic acid;
4-(3-(3-(3-chloro-4-isopropoxyphenyl)-1,2,4-oxadiazol-5-yl)cyclopentylamino)butanoic acid;
(S)-3-(4-(3-(3-chloro-4-isopropoxyphenyl)-1,2,4-oxadiazol-5-yl)phenoxy)propane-1,2-diol;
4-(3-(3-chloro-4-isopropoxyphenyl)-1,2,4-oxadiazol-5-yl)benzenesulfonamide;
tert-butyl 3,3′-(4-(3-(3-chloro-4-isopropoxyphenyl)-1,2,4-oxadiazol-5-yl)phenylsulfonylazanediyl)dipropanoate;
tert-butyl 3-(4-(3-(3-chloro-4-isopropoxyphenyl)-1,2,4-oxadiazol-5-yl)phenylsulfonamido)propanoate;
3-(4-(3-(3-chloro-4-isopropoxyphenyl)-1,2,4-oxadiazol-5-yl)phenylsulfonamido)propanoic acid;
2,2′-(4-(3-(3-chloro-4-isopropoxyphenyl)-1,2,4-oxadiazol-5-yl)phenylsulfonylazanediyl)diacetic acid;
tert-butyl 2,2′-(4-(3-(3-chloro-4-isopropoxyphenyl)-1,2,4-oxadiazol-5-yl)phenylsulfonylazanediyl)diacetate;
tert-butyl 2-(4-(3-(3-chloro-4-isopropoxyphenyl)-1,2,4-oxadiazol-5-yl)phenylsulfonamido)acetate;
2-(4-(3-(3-chloro-4-isopropoxyphenyl)-1,2,4-oxadiazol-5-yl)phenylsulfonamido)acetic acid;
tert-butyl 2-(5-(3-(3-chloro-4-isopropoxyphenyl)-1,2,4-oxadiazol-5-yl)-3,4-dihydroisoquinolin-2(1H)-yl)acetate;
tert-butyl 5-(3-(3-chloro-4-isopropoxyphenyl)-1,2,4-oxadiazol-5-yl)-3,4-dihydroisoquinoline-2(1H)-carboxylate;
3-(3-chloro-4-isopropoxyphenyl)-5-(1,2,3,4-tetrahydroisoquinolin-5-yl)-1,2,4-oxadiazole;
2-(5-(3-(3-chloro-4-isopropoxyphenyl)-1,2,4-oxadiazol-5-yl)-3,4-dihydroisoquinolin-2(1H)-yl)acetic acid;
tert-butyl 3-(5-(3-(3-chloro-4-isopropoxyphenyl)-1,2,4-oxadiazol-5-yl)-3,4-dihydroisoquinolin-2(1H)-yl)propanoate;
3-(5-(3-(3-chloro-4-isopropoxyphenyl)-1,2,4-oxadiazol-5-yl)-3,4-dihydroisoquinolin-2(1H)-yl)propanoic acid;
2-[3-(3-chloro-4-isopropoxy-phenyl)-[1,2,4]oxadiazol-5-yl]-3-methyl-5,6,7,8-tetrahydro-imidazo[1,2-a]pyrazine;
1-{2-[3-(3-chloro-4-isopropoxy-phenyl)-[1,2,4]oxadiazol-5-yl]-3-methyl-5,6-dihydro-8H-imidazo[1,2-a]pyrazin-7-yl}-ethanone;
{2-[3-(3-chloro-4-isopropoxy-phenyl)-[1,2,4]oxadiazol-5-yl]-3-methyl-5,6-dihydro-8H-imidazo[1,2-a]pyrazin-7-yl}-acetic acid tert-butyl ester;
{2-[3-(3-chloro-4-isopropoxy-phenyl)-[1,2,4]oxadiazol-5-yl]-3-methyl-5,6-dihydro-8H-imidazo[1,2-a]pyrazin-7-yl}-acetic acid;
3-[3-(3-chloro-4-isopropoxy-phenyl)-[1,2,4]oxadiazol-5-yl]-2-methyl-imidazo[1,2-a]pyrazine;
3-(3-chloro-4-isopropoxyphenyl)-5-(4-((2,2-dimethyl-1,3-dioxolan-4-yl)methoxy)phenyl)-1,2,4-oxadiazole;
2-(4-(3-(3-chloro-4-isopropoxyphenyl)-1,2,4-oxadiazol-5-yl)phenoxy)acetic acid;
1-(4-(3-(3-cyano-4-isopropoxyphenyl)-1,2,4-oxadiazol-5-yl)benzyl)azetidine-3-carboxylic acid;
1-(4-(3-(3-chloro-4-isopropoxyphenyl)-1,2,4-oxadiazol-5-yl)phenyl)cyclopropanecarbonitrile;
1-(4-(3-(3-chloro-4-isopropoxyphenyl)-1,2,4-oxadiazol-5-yl)phenyl)cyclopropanecarbaldehyde;
3-((1-(4-(3-(3-chloro-4-isopropoxyphenyl)-1,2,4-oxadiazol-5-yl)phenyl)cyclopropyl)methylamino)propanoic acid;
N-(4-(3-(3-chloro-4-isopropoxyphenyl)-1,2,4-oxadiazol-5-yl)benzyl)-1-(2,2-dimethyl-1,3-dioxolan-4-yl)methanamine;
3-(4-(3-(3-chloro-4-isopropoxyphenyl)-1,2,4-oxadiazol-5-yl)benzylamino)propane-1,2-diol;
(Z)-methyl 3-(4-(3-(3-chloro-4-isopropoxyphenyl)-1,2,4-oxadiazol-5-yl)phenyl)acrylate;
trans-methyl 2-(4-(3-(3-chloro-4-isopropoxyphenyl)-1,2,4-oxadiazol-5-yl)phenyl)cyclopropanecarboxylate;
trans-2-(4-(3-(3-chloro-4-isopropoxyphenyl)-1,2,4-oxadiazol-5-yl)phenyl)cyclopropanecarboxylic acid;
tert-butyl 5-(3-(3-chloro-4-isopropoxyphenyl)-1,2,4-oxadiazol-5-yl)isoindoline-2-carboxylate;
3-(3-chloro-4-isopropoxyphenyl)-5-(isoindolin-5-yl)-1,2,4-oxadiazole;
methyl 3-(5-(3-(3-chloro-4-isopropoxyphenyl)-1,2,4-oxadiazol-5-yl)isoindolin-2-yl)propanoate;
3-(5-(3-(3-chloro-4-isopropoxyphenyl)-1,2,4-oxadiazol-5-yl)isoindolin-2-yl)propanoic acid;
(Z)-methyl 3-(4-(3-(3-chloro-4-isopropoxyphenyl)-1,2,4-oxadiazol-5-yl)phenyl)acrylate;
(Z)-3-(4-(3-(3-chloro-4-isopropoxyphenyl)-1,2,4-oxadiazol-5-yl)phenyl)acrylic acid;
3-(3-chloro-4-(3-(3-chloro-4-isopropoxyphenyl)-1,2,4-oxadiazol-5-yl)phenylamino)cyclobutanecarboxylic acid;
3-(4-(3-(3-chloro-4-isopropoxyphenyl)-1,2,4-oxadiazol-5-yl)phenylamino)cyclobutanecarboxylic acid;
1-(4-(3-(4-(4-fluoropiperidin-1-yl)-3-(trifluoromethyl)phenyl)-1,2,4-oxadiazol-5-yl)benzyl)azetidine-3-carboxylic acid;
of 5-((4-(3-(4-(4-fluoropiperidin-1-yl)-3-(trifluoromethyl)phenyl)-1,2,4-oxadiazol-5-yl)benzylamino)methyl)isoxazol-3-ol;
2-((4-(3-(4-isopropoxy-3-(trifluoromethyl)phenyl)-1,2,4-oxadiazol-5-yl)phenoxy)methyl)morpholine;
(1R,3S)-3-(4-(3-(4-((S)-tetrahydrofuran-3-yloxy)-3-(trifluoromethyl)phenyl)-1,2,4-oxadiazol-5-yl)phenylamino)cyclopentanecarboxylic acid;
(1R,3S)-3-(4-(3-(4-(4-fluoropiperidin-1-yl)-3-(trifluoromethyl)phenyl)-1,2,4-oxadiazol-5-yl)phenylamino)cyclopentanecarboxylic acid;
(1R,3S)-3-(4-(3-(4-(4,4-difluoropiperidin-1-yl)-3-(trifluoromethyl)phenyl)-1,2,4-oxadiazol-5-yl)phenylamino)cyclopentanecarboxylic acid;
(1S,3S)-3-(4-(3-(4-(4-fluoropiperidin-1-yl)-3-(trifluoromethyl)phenyl)-1,2,4-oxadiazol-5-yl)phenylamino)cyclopentanecarboxylic acid;
(1R,3R)-3-(4-(3-(4-(4-fluoropiperidin-1-yl)-3-(trifluoromethyl)phenyl)-1,2,4-oxadiazol-5-yl)phenylamino)cyclopentanecarboxylic acid;
(1S,3R)-3-(4-(5-(3-cyano-4-(4-fluoropiperidin-1-yl)phenyl)-1,2,4-oxadiazol-3-yl)-2-(trifluoromethyl)phenylamino)cyclopentanecarboxylic acid;
(1S,3R)-3-(4-(5-(3-cyano-4-(4-fluoropiperidin-1-yl)phenyl)-1,2,4-oxadiazol-3-yl)phenylamino)cyclopentanecarboxylic acid;
5-[3-(4-Fluoro-phenyl)-[1,2,4]oxadiazol-5-yl]-2-(4-fluoro-piperidin-1-yl)-benzonitrile;
5-[3-(4-Fluoro-3-trifluoromethyl-phenyl)-[1,2,4]oxadiazol-5-yl]-2-(4-fluoro-piperidin-1-yl)-benzonitrile;
(1R,3S)-3-[4-(5-biphenyl-2-yl-[1,2,4]oxadiazol-3-yl)-2-methyl-phenylamino]-cyclopentanecarboxylic acid;
(1R,3S)-3-[4-(5-biphenyl-3-yl-[1,2,4]oxadiazol-3-yl)-2-methyl-phenylamino]-cyclopentanecarboxylic acid;
(1R,3S)-3-{4-[5-(4-cyclohexyl-phenyl)-[1,2,4]oxadiazol-3-yl]-2-methyl-phenylamino}-cyclopentanecarboxylic acid; or
(1R,3S)-3-(4-(5-(4-isobutylphenyl)-1,2,4-oxadiazol-3-yl)phenylamino)cyclopentanecarboxylic acid.

13. A compound of Formula (III) 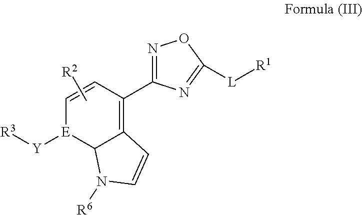
pharmaceutically acceptable salts, biologically active metabolites, solvates, hydrates, prodrugs, enantiomers or stereoisomers thereof, wherein
E is CH or N;
Y is a bond;
L is a bond;
R¹ is optionally substituted aryl;
R² is H;
R³ is H; and
R⁶ is H or optionally substituted (C₁-C₃)alkyl.

14. A compound of Formula (IV): 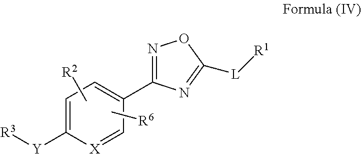
or a pharmaceutically acceptable salt, solvate, hydrate, metabolite, prodrug, enantiomer or stereoisomer thereof,
wherein:
X is N or CR⁴;
L is a bond, —CH₂CH₂—, (C₃-C₆)cycloalkyl, or —CHR⁵;
Y is —O—, —NR⁷— or —C(R⁷)(R^{7′})—;
R¹ is optionally substituted aryl, optionally substituted heteroaryl, optionally substituted heterocyclyl, optionally substituted —(C₁-C₆)alkyl-O—(C₁-C₃)alkyl, optionally substituted —(C₁-C₆)alkyl-O—(C₁-C₆)alkyl-O—(C₁-C₃)alkyl, optionally substituted —(C₁-C₆)alkyl-O-aryl, alkylsulfanylalkyl, unsubstituted (C₂-C₅)alkyl, substituted (C₁-C₆)alkyl, —COR¹¹, optionally substituted —O—(C₁-C₃)alkyl, —N(R⁷)(R⁸), —N(R⁷)SO₂—R¹¹ or optionally substituted (C₃-C₆)cycloalkyl, and wherein R¹ is not substituted cyclopentathiophene, halothiophene, substituted indan or substituted chromenone;
R² and R⁶ may be the same or different and are independently H, —(C₁-C₄)alkyl, —O—(C₁-C₃)alkyl, —CF₃, —CN, halo or —COO—(C₁-C₄)alkyl;
R³ is optionally substituted aryl, optionally substituted heterocyclyl, optionally substituted heteroaryl, optionally substituted (C₃-C₆)cycloalkyl, —(CH₂)ₙ—R¹¹, —CO—OR¹¹, —CO—R¹¹, —CON(R⁷)(R¹¹), —N(R⁷)(R¹¹), —SOR¹¹, —SO₂R¹¹ and optionally substituted straight or branched (C₁-C₈)alkyl chain optionally including —CO—, —COO—, —SO—, —SO₂—, —CONH—, —NHCO—, —N— or —O— groups embedded within the alkyl chain; and when Y is O, R³ is not alkyldiazeapane, —C(CH₃)₂COOCH₂CH₃ or —CH₂CH₂N(CH₂CH₃)₂, and when Y is —CH₂—, R³ is not —CH₂COOH;
or Y is a bond and R³ is optionally substituted morpholino;
R⁴ is H, —(C₁-C₄)alkyl, —O—(C₁-C₃)alkyl, —CF₃, —CN or halo;
R⁵ is H, O—(C₁-C₃)alkyl or (C₁-C₃)alkyl;
each occurrence of R⁷ or R^{7′} is independently H or optionally substituted (C₁-C₃)alkyl;
R⁸ is H, optionally substituted CH₃, or —COR¹¹;
R¹¹ is hydrogen, optionally substituted (C₁-C₃)alkyl, optionally substituted alkynyl, optionally substituted aryl, optionally substituted heteroaryl, optionally substituted heterocyclyl or optionally substituted (C₃-C₆)cycloalkyl; and
n is 1, 2, 3 or 4;
provided that
R¹ is not optionally substituted furanyl or —C(O)-optionally substituted furanyl;
R³ is not optionally substituted quinolinyl;
R¹¹ is not optionally substituted cyclopropyl, optionally substituted cyclohexyl, optionally substituted furanyl, optionally substituted imidazolyl, optionally substituted indolyl, optionally substituted naphthyl, optionally substituted piperazinyl, optionally substituted pyrazolyl, optionally substituted pyridazinyl or optionally substituted quinolinyl;
R¹ is not substituted by —C(O)-cyclopentyl, optionally substituted cyclopentyl, —C(O)-cyclobutyl, cyclobutyl, —C(O)-cyclohexyl or optionally substituted cyclohexyl;
R³ is not substituted by —C(O)-cyclopropyl;
when R³ is CH₃ or 4-chlorophenylmethyl, L-R¹ is not cyclopropyl, cyclopentyl, optionally substituted cyclohexyl, —CH₂-cyclohexyl, —NH-cyclohexyl, —CH₂CH₂-cyclohexyl or optionally substituted pyrazolyl;
when Y is O, R³ is not —(C₀-C₄)alkyl-optionally substituted isoxazolyl or optionally substituted pyrazolyl;
when L is (C₁-C₃)alkyl, R¹ is not optionally substituted isoxazolyl;
when L is a bond, R¹ is not optionally substituted cyclobutyl, optionally substituted cyclohexyl, optionally substituted naphthyl, —CH₂-optionally substituted naphthyl, —CH₂—O-optionally substituted naphthyl, optionally substituted pyrazolyl or tetrahydrobenzofuranyl;
provided the compound is not 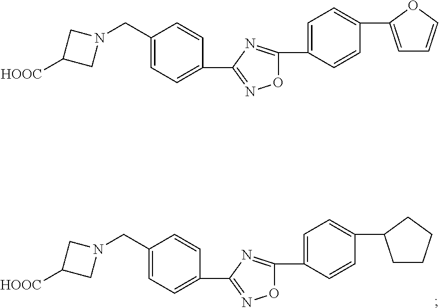
provided the compound is not 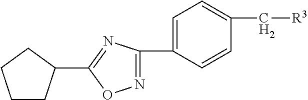
wherein R³ is optionally substituted piperazinyl or optionally substituted phenyl;
provided the compound is not 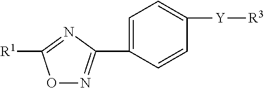
wherein R¹ is optionally substituted pyridine or 3-chlorophenyl and —Y—R³ is
—NH—C(O)-optionally substituted phenyl;
—O-optionally substituted pyridinyl;
—NH—C(O)—OCH₃;
—CH₂-optionally substituted piperazinyl;
—O-optionally substituted (C₁-C₉)alkyl;
—CH₂-morpholinyl; or
—O—C(O)-optionally substituted pyridinyl;
provided the compound is not 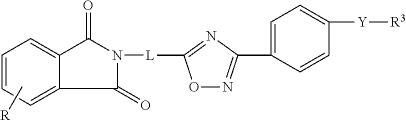
wherein
L is CH₂, CH(CH₃) or CH₂CH₂;
Y is O or CH₂;
R² is H or OCH₃;
R³ is CH₃ or OCF₃; and
R is H or NO₂;
provided the compound is not 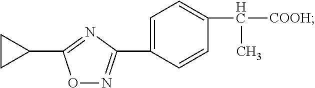
provided the compound is not 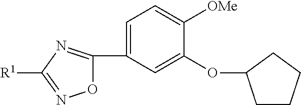
wherein R¹ is phenyl, 4-chlorophenyl, piperidinyl or thienyl.

15. The compound of claim 14 wherein
R¹ is optionally substituted phenyl, optionally substituted tetrahydrobenzofuranyl, optionally substituted furanyl, optionally substituted 2,3-dihydroisoindolyl, optionally substituted isoindolinyl, optionally substituted imidazolyl, optionally substituted 5,6-dihydro imidazo[1,2-a]pyrazinyl, optionally substituted imidazo[1,2-a]pyrazinyl, optionally substituted indolyl, optionally substituted isoxazolyl, optionally substituted pyrazolyl, optionally substituted pyridinyl, optionally substituted pyrimidinyl, optionally substituted pyrrolidinyl, optionally substituted 1,2,3,4-tetrahydroisoquinolinyl, optionally substituted quinolinyl, optionally substituted 3,4-dihydroquinolinyl, optionally substituted 3,4-dihydroisoquinolinyl, optionally substituted 5,6,7,8-tetrahydroimidazo[1,2-a]pyrazinyl, optionally substituted pyrrolyl, optionally substituted pyrrolo[2,3-b]pyridinyl, optionally substituted quinolinyl, optionally substituted thiazolyl, optionally substituted thienyl, optionally substituted —(C₁-C₆)alkyl-O—(C₁-C₃)alkyl, optionally substituted —(C₁-C₆)alkyl-O—(C₁-C₆)alkyl-O—(C₁-C₃)alkyl, optionally substituted —(C₁-C₆)alkyl-O-phenyl, unsubstituted (C₂-C₅)alkyl, substituted (C₁-C₆)alkyl, —COR¹¹, optionally substituted —O—(C₁-C₃)alkyl, —N(R⁷)(R⁸), —N(R⁷)SO₂—R¹¹ or optionally substituted (C₃-C₆)cycloalkyl;
R² and R⁶ may be the same or different and are independently H, —(C₁-C₄)alkyl, —O—(C₁-C₃)alkyl, —CF₃, —CN, Cl, or F.

16. The compound of claim 15 wherein
L is a bond, —CH₂CH₂—, or —CHR⁵;
Y is —O—, —NR⁷— or —C(R⁷)(R^{7′})—;
R¹ is optionally substituted phenyl, optionally substituted furanyl, optionally substituted isoindolinyl, optionally substituted imidazolyl, optionally substituted imidazo[1,2-a]pyrazinyl, optionally substituted indolyl, optionally substituted isoxazolyl, optionally substituted pyrazolyl, optionally substituted pyridinyl, optionally substituted pyrimidinyl, optionally substituted pyrrolidinyl, optionally substituted quinolinyl, optionally substituted pyrrolyl, optionally substituted pyrrolo[2,3-b]pyridinyl, optionally substituted quinolinyl, optionally substituted thiazolyl, optionally substituted thienyl, optionally substituted —(C₁-C₆)alkyl-O—(C₁-C₃)alkyl, optionally substituted —(C₁-C₆)alkyl-O-phenyl, unsubstituted (C₂-C₅)alkyl, substituted (C₁-C₆)alkyl, —COR¹¹, optionally substituted —O—(C₁-C₃)alkyl, —N(R⁷)(R⁸), —N(R⁷)SO₂—R¹¹ or optionally substituted (C₃-C₆)cycloalkyl;
R² and R⁶ may be the same or different and are independently H, —(C₁-C₄)alkyl, —O—(C₁-C₃)alkyl, —CF₃, —CN, Cl or F;
R³ is optionally substituted phenyl, optionally substituted piperidinyl, optionally substituted furanyl, optionally substituted pyrimidinyl, optionally substituted pyridinyl, optionally substituted (C₃-C₆)cycloalkyl, —(CH₂)ₙ—R¹¹, —CO—OR¹¹, —CO—R¹¹, —CON(R⁷)(R¹¹), —N(R⁷)(R¹¹), —SOR¹¹, —SO₂R¹¹ and optionally substituted straight or branched (C₁-C₈)alkyl chain.

17. The compound of claim 16 wherein R¹ is optionally substituted phenyl, optionally substituted furanyl, optionally substituted indolyl, optionally substituted isoxazolyl, optionally substituted pyrazolyl, optionally substituted pyridinyl, optionally substituted pyrimidinyl, optionally substituted pyrrolyl, unsubstituted (C₂-C₅)alkyl, substituted (C₁-C₆)alkyl, —COR¹¹, —N(R⁷)(R⁸), optionally substituted —O—(C₁-C₃)alkyl, or optionally substituted (C₃-C₆)cycloalkyl;
R² and R⁶ may be the same or different and are independently H, —(C₁-C₄)alkyl, —CF₃, Cl or F;
R³ is optionally substituted phenyl, optionally substituted piperidinyl optionally substituted pyrimidinyl, optionally substituted pyridinyl, optionally substituted (C₃-C₆)cycloalkyl, —(CH₂)ₙ—R¹¹, optionally substituted straight or branched (C₁-C₈)alkyl chain
or 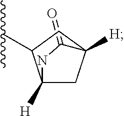
or
—Y—R³ is

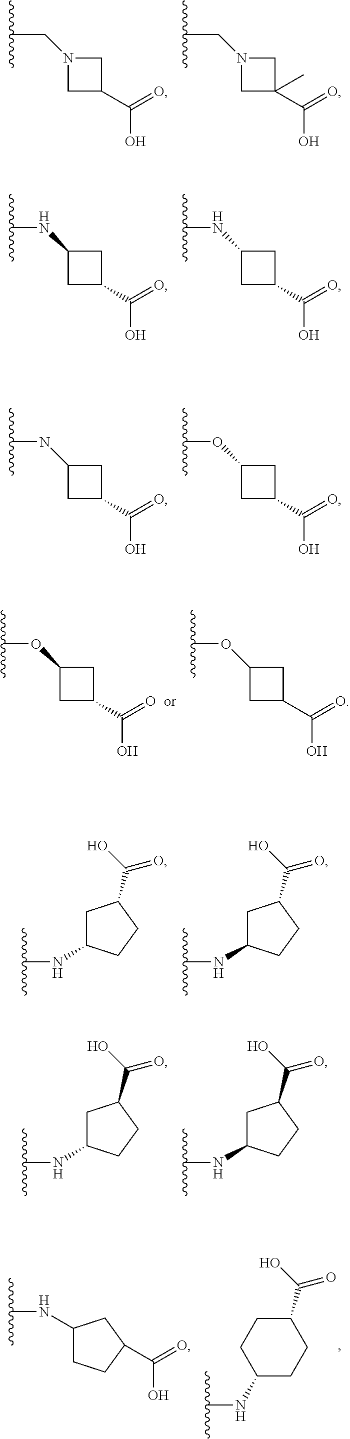

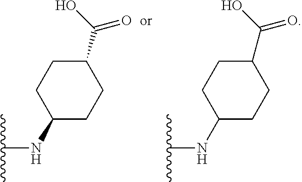

18. The compound of claim 17 wherein R¹ is optionally substituted by one or more substituents independently selected from Br, Cl, F, CF₃, CN, oxo, —C(═O)H, —N(R⁹)₂, optionally substituted (C₁-C₆)alkyl, optionally substituted (C₂-C₆)alkenyl, optionally substituted (C₂-C₆)alkynyl, optionally substituted azabicyclo[2.2.1]heptanyl, optionally substituted (C₃-C₆)cycloalkyl, —C(R⁹)₂-optionally substituted (C₃-C₆)cycloalkyl, —C(R⁹)₂-optionally substituted azetidinyl, —CR⁹₂-optionally substituted piperidinyl, —C(R⁹)₂-optionally substituted pyrrolidinyl, —C(R⁹)₂—N(R⁹)₂, —C(O)-optionally substituted (C₁-C₆)alkyl, —C(O)—NR⁹—(C₁-C₆)alkyl, —C(O)—O-optionally substituted (C₁-C₆)alkyl, —C(R⁹)₂—C(O)—O-optionally substituted (C₁-C₆)alkyl, —NR⁹-optionally substituted (C₃-C₆)cycloalkyl, —NR⁹-optionally substituted azetidinyl, —NR₉-furanyl, —NR⁹-optionally substituted pyrrolidinyl, —NR⁹—C(O)—O-optionally substituted (C₁-C₃)alkyl, —NR⁹-optionally substituted (C₁-C₆)alkyl, —NR⁹-optionally substituted (C₃-C₆)cycloalkyl, —NR⁹—C(O)-azetidinyl, —NR⁹—C(O)-furanyl, —NR⁹—C(O)-pyridinyl, —NR⁹—C(O)-optionally substituted pyrrolidinyl, —NR⁹—S(O)₂-optionally substituted phenyl, —O-optionally substituted (C₁-C₆)alkyl, —O-deuterated —(C₂-C₆)alkyl, —O-optionally substituted (C₂-C₆)alkenyl, —O-optionally substituted (C₃-C₆)cycloalkyl, —O-1H-benzo[d][1,2,3]triazolyl, —S(O)₂—N(R⁹)₂, —S(O)₂—NR⁹-optionally substituted (C₁-C₄)alkyl, optionally substituted azetidinyl, optionally substituted piperidinyl, optionally substituted pyridinyl, optionally substituted pyrrolidinyl, optionally substituted 1,2,4 oxadiazolyl, optionally substituted pyrrolidinyl, optionally substituted tetrazolyl, 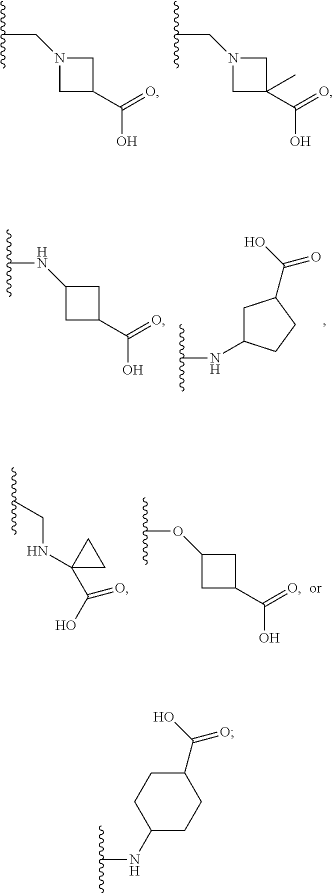
and
wherein each R⁹ is independently selected from H or optionally substituted (C₁-C₆)alkyl.

19. The compound of claim 18 wherein each substituent or optional substituent is independently one or more R¹⁰ groups wherein R¹⁰ is optionally substituted alkyl, alkenyl, optionally substituted alkoxy groups, alkoxyalkoxy, alkoxyalkyl, alkoxycarbonyl, alkoxycarbonylheterocycloalkoxy, alkyl, alkylamino, alkylcarbonyl, alkylester, alkyl-O—C(O)—, alkyl-heterocyclyl, alkyl-cycloalkyl, alkyl-nitrile, alkylsulfonyl, alkynyl, amido groups, optionally substituted amino, aminoalkyl, aminoalkoxy, aminocarbonyl, optionally substituted azabicyclo[2.2.1]heptanyl, carbonitrile, carbonylalkoxy, carboxamido, CF₃, CN, —C(O)OH, —C(O)H, —C(O)—C(CH₃)₃, —OH, —C(O)O-alkyl, —C(O)O— optionally substituted cycloalkyl, —C(O)O-heterocyclyl, —C(O)-alkyl, —C(O)— optionally substituted cycloalkyl, —C(O)-heterocyclyl, CN, optionally substituted cycloalkyl, dialkylamino, dialkylaminoalkoxy, dialkylaminocarbonylalkoxy, dialkylaminocarbonyl, dialkylaminosulfonyl, —C(O)—OR^{a}, halogen, optionally substituted heterocyclyl, optionally substituted heterocyclylalkyl, optionally substituted heterocyclyloxy, hydroxy, hydroxyalkyl, nitro, oxo, optionally substituted phenyl, —SO₂CH₃, —SO₂CF₃, sulfonyl, tetrazolyl, thienylalkoxy, trifluoromethylcarbonylamino, trifluoromethylsulfonamido, heterocyclylalkoxy, heterocyclyl-S(O)ₚ, optionally substituted cycloalkyl-S(O)ₚ, optionally substituted alkyl-S—, optionally substituted heterocyclyl-S, heterocycloalkyl, cycloalkylalkyl, heterocycolthio, cycloalkylthio, N-alkylamino and N,N-dialkylamino where R^{a} is optionally substituted alkyl, optionally substituted heterocycloalkyl, or optionally substituted heterocyclyl and p is 1 or 2.

20. The compound according to claim 19 wherein —Y—R³ is

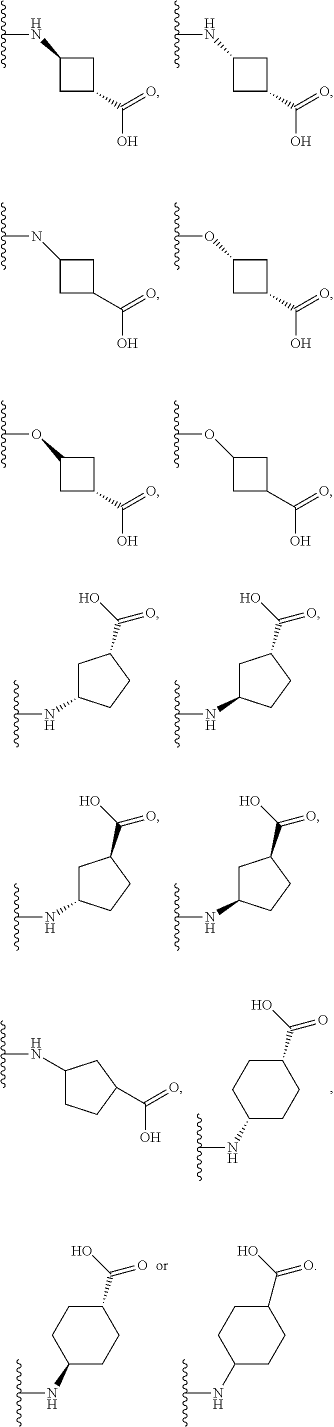

21. The compound according to claim 20 wherein the compound is
3-{3-chloro-4-[5-(5-chloro-6-isopropoxy-pyridin-3-yl)-[1,2,4]oxadiazol-3-yl]-phenoxy}-cyclobutanecarboxylic acid;
(1R,3S)-3-(4-(5-(5-chloro-6-isopropoxypyridin-3-yl)-1,2,4-oxadiazol-3-yl)phenylamino)cyclopentanecarboxylic acid;
(1S,4R)-2-(3-(3-(5-chloro-6-isopropoxypyridin-3-yl)-1,2,4-oxadiazol-5-yl)-2-methylphenyl)-2-azabicyclo[2.2.1]heptan-3-one;
(1R,3S)-3-(4-(3-(5-chloro-6-isopropoxypyridin-3-yl)-1,2,4-oxadiazol-5-yl)-2-methylphenylamino)cyclopentanecarboxylic acid;
(1R,3S)-3-(4-(3-(5-chloro-6-isopropoxypyridin-3-yl)-1,2,4-oxadiazol-5-yl)-3-(trifluoromethyl)phenylamino)cyclopentanecarboxylic acid;
1-amino-3-(3-chloro-4-(5-(5-chloro-6-isopropoxypyridin-3-yl)-1,2,4-oxadiazol-3-yl)phenoxy)cyclopentanecarboxylic acid;
3-{3-chloro-4-[5-(5-chloro-6-isopropoxy-pyridin-3-yl)-[1,2,4]oxadiazol-3-yl]-phenoxy}-cyclobutanecarboxylic acid;
4-{3-chloro-4-[5-(5-chloro-6-isopropoxy-pyridin-3-yl)-[1,2,4]oxadiazol-3-yl]-phenoxy}-cyclohexanecarboxylic acid;
3-{3-chloro-4-[3-(5-chloro-6-isopropoxy-pyridin-3-yl)-[1,2,4]oxadiazol-5-yl]-phenoxy}-cyclobutanecarboxylic acid;
3-{3-chloro-4-[3-(5-chloro-6-isopropoxy-pyridin-3-yl)-[1,2,4]oxadiazol-5-yl]-phenoxy}-cyclobutanecarboxylic acid;
3-{3-chloro-4-[5-(5-chloro-6-isopropoxy-pyridin-3-yl)-[1,2,4]oxadiazol-3-yl]-phenoxy}-cyclohexanecarboxylic acid;
4-{3-chloro-4-[5-(5-chloro-6-isopropoxy-pyridin-3-yl)-[1,2,4]oxadiazol-3-yl]-phenoxy}-cyclohexanecarboxylic acid;
cis-3-{3-chloro-4-[5-(5-chloro-6-isopropoxy-pyridin-3-yl)-[1,2,4]oxadiazol-3-yl]-phenoxy}-cyclopentanecarboxylic acid;
trans-3-{3-chloro-4-[5-(5-chloro-6-isopropoxy-pyridin-3-yl)-[1,2,4]oxadiazol-3-yl]-phenoxy}-cyclopentanecarboxylic acid;
or
(1R,3S)-3-(4-(5-(5-bromo-6-isopropoxypyridin-3-yl)-1,2,4-oxadiazol-3-yl)phenylamino)cyclopentanecarboxylic acid.

22. A compound according to Formula (V) 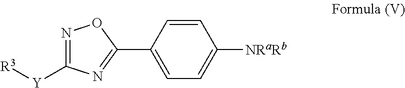
Y is a bond
R³ is -3-((1aS,5aR)-1,1,2-trimethyl-1,1a,5,5a-tetrahydro-3-thia-cyclopropa[a]pentalenyl, or optionally substituted thienyl;
R^{a} is H or optionally substituted (C₁-C₆)alkyl
R^{b} is H, optionally substituted (C₁-C₆)alkyl or optionally substituted (C₃-C₆)cycloalkyl.

23. The compound of claim 22 wherein the compound is
(1R,3S)-3-(4-(3-(4-phenyl-5-(trifluoromethyl)thiophen-2-yl)-1,2,4-oxadiazol-5-yl)phenylamino)cyclopentanecarboxylic acid; or
(S)-3-{4-[3-((1aS,5aR)-1,1,2-trimethyl-1,1a,5,5a-tetrahydro-3-thia cyclopropa[a]pentalen-4-yl)-[1,2,4]oxadiazol-5-yl]-phenylamino}-cyclopentanecarboxylic acid.

24. A pharmaceutical composition comprising a compound according to claim 1 or 14 or a pharmaceutically acceptable salt, solvate, hydrate, metabolite, prodrug, enantiomer or stereoisomer thereof and a pharmaceutically acceptable diluent or carrier.

25. The use of one or more compounds according to claim 1 or 14 or a pharmaceutically acceptable salt, solvate, hydrate, metabolite, prodrug or stereoisomer thereof for the manufacture of a medicament for treating an immune disorder.

26. The use according claim 25 wherein the immune disorder is active chronic hepatitis, Addison's Disease, ankylosing spondylitis, anti-phospholipid syndrome, asthma, atopic allergy, autoimmune atrophic gastritis, achlorhydra autoimmune, Celiac Disease, Crohn's Disease, Cushing's Syndrome, dermatomyositis, Goodpasture's Syndrome, Grave's Disease, Hashimoto's thyroiditis, idiopathic adrenal atrophy, idiopathic thrombocytopenia, juvenile rheumatoid arthritis, Lambert-Eaton Syndrome, lupoid hepatitis, lupus, mixed connective tissue disease, multiple sclerosis, pemphigoid, pemphigus vulgaris, pernicious anemia, phacogenic uveitis, polyarteritis nodosa, primary biliary cirrhosis, primary sclerosing cholangitis, psoriasis, Raynauds, Reiter's Syndrome, relapsing polychondritis, Schmidt's Syndrome, Sjogren's Syndrome, sympathetic ophthalmia, Takayasu's Arteritis, temporal arteritis, thyrotoxicosis, rheumatoid arthritis, Type B Insulin Resistance, ulcerative colitis, or Wegener's granulomatosis.

27. A use of one or more compounds according to claim 1 or 14 or a pharmaceutically acceptable salt, solvate, hydrate, metabolite, prodrug, enantiomer or stereoisomer thereof for the manufacture of a medicament for treating a central nervous system disorder.

28. A use of one or more compounds according to claim 1 or 14 or a pharmaceutically acceptable salt, solvate, hydrate, metabolite, prodrug, enantiomer or stereoisomer thereof for the manufacture of a medicament for treating multiple sclerosis.

29. A use of one or more compounds according to claim 1 or 14 or a pharmaceutically acceptable salt, solvate, hydrate, metabolite, prodrug, enantiomer or stereoisomer thereof for the manufacture of a medicament for treating rheumatoid arthritis.

30. A pharmaceutical composition comprising one or more compounds according to Formula (I), (Ia), (II), (III), (IV), or (V) or pharmaceutically acceptable salts, solvates, hydrates, metabolites, prodrugs or stereoisomers thereof and a pharmaceutically acceptable diluent or carrier. In a preferred aspect, the invention provides a pharmaceutical composition wherein the compound or compounds are present in a therapeutically effective amount.

31. A packaged pharmaceutical comprising a one or more compounds according to Formula (I), (Ia), (II), (III), (IV), or (V) or pharmaceutically acceptable salts, solvates, hydrates, metabolites, prodrugs or stereoisomers thereof and instructions for use. In one embodiment, the invention provides a packaged pharmaceutical wherein the compound or compounds are present in a therapeutically effective amount. In another embodiment, the invention provides a packaged pharmaceutical wherein the compound or compounds are present in a prophylactically effective amount.
